SEQUENCE LISTING (1245 sequences)

SEQ ID NO: 1 - protein, 9 aa, Artificial Sequence
  Synthetic polypeptide
YMMDDLELI

SEQ ID NO: 2 - protein, 9 aa, Artificial Sequence
  Synthetic polypeptide
SMMPPPPAL

SEQ ID NO: 3 - protein, 9 aa, Artificial Sequence
  Synthetic polypeptide
FMNSTVFHV

SEQ ID NO: 4 - protein, 11 aa, Artificial Sequence
  Synthetic polypeptide
SLVRLSSCVPV

SEQ ID NO: 5 - protein, 8 aa, Artificial Sequence
  Synthetic polypeptide
FLQEVFQA

SEQ ID NO: 6 - protein, 9 aa, Artificial Sequence
  Synthetic polypeptide
KLFEKKYSV

SEQ ID NO: 7 - protein, 9 aa, Artificial Sequence
  Synthetic polypeptide
SPAIPPRPL

SEQ ID NO: 8 - protein, 9 aa, Artificial Sequence
  Synthetic polypeptide
KPIAGRHTL

SEQ ID NO: 9 - protein, 9 aa, Artificial Sequence
  Synthetic polypeptide
FLGHHSFSV

SEQ ID NO: 10 - protein, 9 aa, Artificial Sequence
  Synthetic polypeptide
FLIDDNFKV

SEQ ID NO: 11 - protein, 9 aa, Artificial Sequence
  Synthetic polypeptide
FLMDGFDEL

SEQ ID NO: 12 - protein, 9 aa, Artificial Sequence
  Synthetic polypeptide
FLWNSLLAV

SEQ ID NO: 13 - protein, 10 aa, Artificial Sequence
  Synthetic polypeptide
RPRMRAASPL

SEQ ID NO: 14 - protein, 9 aa, Artificial Sequence
  Synthetic polypeptide
RPVRGRGSL

SEQ ID NO: 15 - protein, 10 aa, Artificial Sequence
  Synthetic polypeptide
RPLCSSLGPL

SEQ ID NO: 16 - protein, 9 aa, Artificial Sequence
  Synthetic polypeptide
FLAPCNFYL

SEQ ID NO: 17 - protein, 11 aa, Artificial Sequence
  Synthetic polypeptide
TAPVASASPKL

SEQ ID NO: 18 - protein, 11 aa, Artificial Sequence
  Synthetic polypeptide
APREGAAATPL

SEQ ID NO: 19 - protein, 9 aa, Artificial Sequence
  Synthetic polypeptide
KMMKILMIK

SEQ ID NO: 20 - protein, 10 aa, Artificial Sequence
  Synthetic polypeptide
LSAPEKITLF

SEQ ID NO: 21 - protein, 9 aa, Artificial Sequence
  Synthetic polypeptide
LSTEVQSLY

SEQ ID NO: 22 - protein, 9 aa, Artificial Sequence
  Synthetic polypeptide
KLSSVVPSV

SEQ ID NO: 23 - protein, 10 aa, Artificial Sequence
  Synthetic polypeptide
SLWSSMPHGV

SEQ ID NO: 24 - protein, 9 aa, Artificial Sequence
  Synthetic polypeptide
TQLARFFPI

SEQ ID NO: 25 - protein, 9 aa, Artificial Sequence
  Synthetic polypeptide
ALQSDVQPV

SEQ ID NO: 26 - protein, 9 aa, Artificial Sequence
  Synthetic polypeptide
SLINIHHRK

SEQ ID NO: 27 - protein, 10 aa, Artificial Sequence
  Synthetic polypeptide
RVPAHASTSL

SEQ ID NO: 28 - protein, 9 aa, Artificial Sequence
  Synthetic polypeptide
IAQPSTSSL

SEQ ID NO: 29 - protein, 9 aa, Artificial Sequence
  Synthetic polypeptide
MLLRLNLRK

SEQ ID NO: 30 - protein, 9 aa, Artificial Sequence
  Synthetic polypeptide
APSWPDRPL

SEQ ID NO: 31 - protein, 9 aa, Artificial Sequence
  Synthetic polypeptide
RLLPYPFHV

SEQ ID NO: 32 - protein, 9 aa, Artificial Sequence
  Synthetic polypeptide
KMLTALPPA

SEQ ID NO: 33 - protein, 9 aa, Artificial Sequence
  Synthetic polypeptide
KIKHGLSEK

SEQ ID NO: 34 - protein, 10 aa, Artificial Sequence
  Synthetic polypeptide
YQMDFHPSPV

SEQ ID NO: 35 - protein, 9 aa, Artificial Sequence
  Synthetic polypeptide
SPRPSACQL

SEQ ID NO: 36 - protein, 9 aa, Artificial Sequence
  Synthetic polypeptide
QPHVPPSTL

SEQ ID NO: 37 - protein, 10 aa, Artificial Sequence
  Synthetic polypeptide
RLYVPLYSSK

SEQ ID NO: 38 - protein, 9 aa, Artificial Sequence
  Synthetic polypeptide
LSSPFREQM

SEQ ID NO: 39 - protein, 10 aa, Artificial Sequence
  Synthetic polypeptide
IQKSWTATTY

SEQ ID NO: 40 - protein, 9 aa, Artificial Sequence
  Synthetic polypeptide
FLDPDIGGV

SEQ ID NO: 41 - protein, 9 aa, Artificial Sequence
  Synthetic polypeptide
FAMAQIQSL

SEQ ID NO: 42 - protein, 9 aa, Artificial Sequence
  Synthetic polypeptide
RPRLPRHCL

SEQ ID NO: 43 - protein, 9 aa, Artificial Sequence
  Synthetic polypeptide
YIMHLWPPI

SEQ ID NO: 44 - protein, 10 aa, Artificial Sequence
  Synthetic polypeptide
FLATSGIDPV

SEQ ID NO: 45 - protein, 9 aa, Artificial Sequence
  Synthetic polypeptide
TLDVELPPV

SEQ ID NO: 46 - protein, 9 aa, Artificial Sequence
  Synthetic polypeptide
TLISMPYHV

SEQ ID NO: 47 - protein, 11 aa, Artificial Sequence
  Synthetic polypeptide
SPMGRKQGGTL

SEQ ID NO: 48 - protein, 10 aa, Artificial Sequence
  Synthetic polypeptide
RPKKSGDMTL

SEQ ID NO: 49 - protein, 9 aa, Artificial Sequence
  Synthetic polypeptide
NMIQVLMSV

SEQ ID NO: 50 - protein, 10 aa, Artificial Sequence
  Synthetic polypeptide
SLYGWYQLCV

SEQ ID NO: 51 - protein, 10 aa, Artificial Sequence
  Synthetic polypeptide
STMRVAVTPK

SEQ ID NO: 52 - protein, 10 aa, Artificial Sequence
  Synthetic polypeptide
RNLKNFLLMK

SEQ ID NO: 53 - protein, 9 aa, Artificial Sequence
  Synthetic polypeptide
SLMEQIPHL

SEQ ID NO: 54 - protein, 10 aa, Artificial Sequence
  Synthetic polypeptide
RTRGVCSVLK

SEQ ID NO: 55 - protein, 9 aa, Artificial Sequence
  Synthetic polypeptide
IMHQYPNFK

SEQ ID NO: 56 - protein, 9 aa, Artificial Sequence
  Synthetic polypeptide
KTVQAEPLI

SEQ ID NO: 57 - protein, 8 aa, Artificial Sequence
  Synthetic polypeptide
KTYMEMHY

SEQ ID NO: 58 - protein, 9 aa, Artificial Sequence
  Synthetic polypeptide
RSVLEEMGL

SEQ ID NO: 59 - protein, 9 aa, Artificial Sequence
  Synthetic polypeptide
ALQEISFWL

SEQ ID NO: 60 - protein, 9 aa, Artificial Sequence
  Synthetic polypeptide
RLFSFPAAK

SEQ ID NO: 61 - protein, 9 aa, Artificial Sequence
  Synthetic polypeptide
KSLPSFLTM

SEQ ID NO: 62 - protein, 10 aa, Artificial Sequence
  Synthetic polypeptide
KANRYFSPNF

SEQ ID NO: 63 - protein, 9 aa, Artificial Sequence
  Synthetic polypeptide
SMASIMETI

SEQ ID NO: 64 - protein, 10 aa, Artificial Sequence
  Synthetic polypeptide
TQGARSSAAF

SEQ ID NO: 65 - protein, 10 aa, Artificial Sequence
  Synthetic polypeptide
FLRLDDLFKL

SEQ ID NO: 66 - protein, 9 aa, Artificial Sequence
  Synthetic polypeptide
SLINLTWTA

SEQ ID NO: 67 - protein, 9 aa, Artificial Sequence
  Synthetic polypeptide
MMIYFDMEV

SEQ ID NO: 68 - protein, 10 aa, Artificial Sequence
  Synthetic polypeptide
LLKETKFITY

SEQ ID NO: 69 - protein, 10 aa, Artificial Sequence
  Synthetic polypeptide
KSFHGLDFGF

SEQ ID NO: 70 - protein, 9 aa, Artificial Sequence
  Synthetic polypeptide
RATFLLALW

SEQ ID NO: 71 - protein, 9 aa, Artificial Sequence
  Synthetic polypeptide
IMMSWMPPL

SEQ ID NO: 72 - protein, 9 aa, Artificial Sequence
  Synthetic polypeptide
KTHPCTMLL

SEQ ID NO: 73 - protein, 9 aa, Artificial Sequence
  Synthetic polypeptide
APLFRASIL

SEQ ID NO: 74 - protein, 9 aa, Artificial Sequence
  Synthetic polypeptide
WLWENHEKL

SEQ ID NO: 75 - protein, 9 aa, Artificial Sequence
  Synthetic polypeptide
RRWECSHRL

SEQ ID NO: 76 - protein, 9 aa, Artificial Sequence
  Synthetic polypeptide
SAFSSLLPL

SEQ ID NO: 77 - protein, 9 aa, Artificial Sequence
  Synthetic polypeptide
FMQFSLFSV

SEQ ID NO: 78 - protein, 9 aa, Artificial Sequence
  Synthetic polypeptide
WMIVTVLPV

SEQ ID NO: 79 - protein, 9 aa, Artificial Sequence
  Synthetic polypeptide
FAFDSPHHY

SEQ ID NO: 80 - protein, 10 aa, Artificial Sequence
  Synthetic polypeptide
GRLRVGLRLL

SEQ ID NO: 81 - protein, 9 aa, Artificial Sequence
  Synthetic polypeptide
FLLTTLLGV

SEQ ID NO: 82 - protein, 9 aa, Artificial Sequence
  Synthetic polypeptide
KRAARLVLR

SEQ ID NO: 83 - protein, 10 aa, Artificial Sequence
  Synthetic polypeptide
VLSVRLPTRK

SEQ ID NO: 84 - protein, 9 aa, Artificial Sequence
  Synthetic polypeptide
RMKHFIYFK

SEQ ID NO: 85 - protein, 10 aa, Artificial Sequence
  Synthetic polypeptide
HRLRSLPRPL

SEQ ID NO: 86 - protein, 10 aa, Artificial Sequence
  Synthetic polypeptide
FMDQEFLSFV

SEQ ID NO: 87 - protein, 9 aa, Artificial Sequence
  Synthetic polypeptide
LLDDSNFKV

SEQ ID NO: 88 - protein, 11 aa, Artificial Sequence
  Synthetic polypeptide
SPPVRSTVCAM

SEQ ID NO: 89 - protein, 9 aa, Artificial Sequence
  Synthetic polypeptide
KADFRTLLK

SEQ ID NO: 90 - protein, 9 aa, Artificial Sequence
  Synthetic polypeptide
FLAVDTQLL

SEQ ID NO: 91 - protein, 10 aa, Artificial Sequence
  Synthetic polypeptide
RVYDPASPQR

SEQ ID NO: 92 - protein, 10 aa, Artificial Sequence
  Synthetic polypeptide
RSLQAHKMAW

SEQ ID NO: 93 - protein, 9 aa, Artificial Sequence
  Synthetic polypeptide
FLSPWPSPA

SEQ ID NO: 94 - protein, 9 aa, Artificial Sequence
  Synthetic polypeptide
KRQKLICQM

SEQ ID NO: 95 - protein, 10 aa, Artificial Sequence
  Synthetic polypeptide
SPPLHLCQPL

SEQ ID NO: 96 - protein, 10 aa, Artificial Sequence
  Synthetic polypeptide
MTIYIFCLHY

SEQ ID NO: 97 - protein, 9 aa, Artificial Sequence
  Synthetic polypeptide
RPACTCISM

SEQ ID NO: 98 - protein, 9 aa, Artificial Sequence
  Synthetic polypeptide
LLLGCLCFI

SEQ ID NO: 99 - protein, 11 aa, Artificial Sequence
  Synthetic polypeptide
RPENSQINSSL

SEQ ID NO: 100 - protein, 9 aa, Artificial Sequence
  Synthetic polypeptide
SLMMIVLTI

SEQ ID NO: 101 - protein, 9 aa, Artificial Sequence
  Synthetic polypeptide
NMMCQHTMI

SEQ ID NO: 102 - protein, 10 aa, Artificial Sequence
  Synthetic polypeptide
YLTKWPKFFL

SEQ ID NO: 103 - protein, 10 aa, Artificial Sequence
  Synthetic polypeptide
YSYPSSLSVF

SEQ ID NO: 104 - protein, 9 aa, Artificial Sequence
  Synthetic polypeptide
TLWSRLVLA

SEQ ID NO: 105 - protein, 9 aa, Artificial Sequence
  Synthetic polypeptide
LTSSQSSWW

SEQ ID NO: 106 - protein, 9 aa, Artificial Sequence
  Synthetic polypeptide
ASLAHSDNF

SEQ ID NO: 107 - protein, 9 aa, Artificial Sequence
  Synthetic polypeptide
AMAQVTHPL

SEQ ID NO: 108 - protein, 10 aa, Artificial Sequence
  Synthetic polypeptide
KMNKILLPWK

SEQ ID NO: 109 - protein, 10 aa, Artificial Sequence
  Synthetic polypeptide
QLRCWNTWAK

SEQ ID NO: 110 - protein, 9 aa, Artificial Sequence
  Synthetic polypeptide
SLVTISRFV

SEQ ID NO: 111 - protein, 10 aa, Artificial Sequence
  Synthetic polypeptide
YSDENMMDPY

SEQ ID NO: 112 - protein, 10 aa, Artificial Sequence
  Synthetic polypeptide
FLALNQLPQV

SEQ ID NO: 113 - protein, 8 aa, Artificial Sequence
  Synthetic polypeptide
KPRPLHAL

SEQ ID NO: 114 - protein, 11 aa, Artificial Sequence
  Synthetic polypeptide
SLLSVGNLIGL

SEQ ID NO: 115 - protein, 10 aa, Artificial Sequence
  Synthetic polypeptide
SQVWTAATLR

SEQ ID NO: 116 - protein, 9 aa, Artificial Sequence
  Synthetic polypeptide
GMVPLIIPV

SEQ ID NO: 117 - protein, 9 aa, Artificial Sequence
  Synthetic polypeptide
TPQDSRQVL

SEQ ID NO: 118 - protein, 9 aa, Artificial Sequence
  Synthetic polypeptide
RAWRRFPLL

SEQ ID NO: 119 - protein, 9 aa, Artificial Sequence
  Synthetic polypeptide
VGMRETTGL

SEQ ID NO: 120 - protein, 9 aa, Artificial Sequence
  Synthetic polypeptide
TSSPRTMSW

SEQ ID NO: 121 - protein, 10 aa, Artificial Sequence
  Synthetic polypeptide
SWMGGLHSFY

SEQ ID NO: 122 - protein, 9 aa, Artificial Sequence
  Synthetic polypeptide
SQKNITPAI

SEQ ID NO: 123 - protein, 9 aa, Artificial Sequence
  Synthetic polypeptide
KADQSESSL

SEQ ID NO: 124 - protein, 9 aa, Artificial Sequence
  Synthetic polypeptide
LTHPAHQPL

SEQ ID NO: 125 - protein, 10 aa, Artificial Sequence
  Synthetic polypeptide
RTLLVTCILY

SEQ ID NO: 126 - protein, 9 aa, Artificial Sequence
  Synthetic polypeptide
RSAFPSRSL

SEQ ID NO: 127 - protein, 9 aa, Artificial Sequence
  Synthetic polypeptide
VVHKKRGLF

SEQ ID NO: 128 - protein, 9 aa, Artificial Sequence
  Synthetic polypeptide
LSWRGASFI

SEQ ID NO: 129 - protein, 10 aa, Artificial Sequence
  Synthetic polypeptide
QSYNTVTRQW

SEQ ID NO: 130 - protein, 9 aa, Artificial Sequence
  Synthetic polypeptide
WTGSCRQGW

SEQ ID NO: 131 - protein, 9 aa, Artificial Sequence
  Synthetic polypeptide
KRAFIHTPR

SEQ ID NO: 132 - protein, 9 aa, Artificial Sequence
  Synthetic polypeptide
LKLCSKVSF

SEQ ID NO: 133 - protein, 9 aa, Artificial Sequence
  Synthetic polypeptide
KVDTHHLQV

SEQ ID NO: 134 - protein, 10 aa, Artificial Sequence
  Synthetic polypeptide
SPSRSTTAPV

SEQ ID NO: 135 - protein, 9 aa, Artificial Sequence
  Synthetic polypeptide
CRREYRVTM

SEQ ID NO: 136 - protein, 10 aa, Artificial Sequence
  Synthetic polypeptide
WSWCGTSQTY

SEQ ID NO: 137 - protein, 9 aa, Artificial Sequence
  Synthetic polypeptide
KPLWRKSPL

SEQ ID NO: 138 - protein, 9 aa, Artificial Sequence
  Synthetic polypeptide
RLSCAPPPI

SEQ ID NO: 139 - protein, 9 aa, Artificial Sequence
  Synthetic polypeptide
LSSWFSPTV

SEQ ID NO: 140 - protein, 9 aa, Artificial Sequence
  Synthetic polypeptide
MSSIWGTMF

SEQ ID NO: 141 - protein, 9 aa, Artificial Sequence
  Synthetic polypeptide
RTRSAWGDW

SEQ ID NO: 142 - protein, 10 aa, Artificial Sequence
  Synthetic polypeptide
RTIMGWTLDF

SEQ ID NO: 143 - protein, 9 aa, Artificial Sequence
  Synthetic polypeptide
ASRPGSFTF

SEQ ID NO: 144 - protein, 10 aa, Artificial Sequence
  Synthetic polypeptide
KSLEGNLETF

SEQ ID NO: 145 - protein, 9 aa, Artificial Sequence
  Synthetic polypeptide
LALPCRSVW

SEQ ID NO: 146 - protein, 10 aa, Artificial Sequence
  Synthetic polypeptide
VLEFSSDRKK

SEQ ID NO: 147 - protein, 10 aa, Artificial Sequence
  Synthetic polypeptide
KAFLPERKCF

SEQ ID NO: 148 - protein, 10 aa, Artificial Sequence
  Synthetic polypeptide
TNTMGGVQGK

SEQ ID NO: 149 - protein, 10 aa, Artificial Sequence
  Synthetic polypeptide
GSHNIKKAWY

SEQ ID NO: 150 - protein, 9 aa, Artificial Sequence
  Synthetic polypeptide
AMAENILAA

SEQ ID NO: 151 - protein, 9 aa, Artificial Sequence
  Synthetic polypeptide
YLGTPTWNC

SEQ ID NO: 152 - protein, 10 aa, Artificial Sequence
  Synthetic polypeptide
RRPLRSWTPR

SEQ ID NO: 153 - protein, 9 aa, Artificial Sequence
  Synthetic polypeptide
RAWRAGMPL

SEQ ID NO: 154 - protein, 10 aa, Artificial Sequence
  Synthetic polypeptide
HSWRFCTHIR

SEQ ID NO: 155 - protein, 10 aa, Artificial Sequence
  Synthetic polypeptide
MPCFTTALLL

SEQ ID NO: 156 - protein, 9 aa, Artificial Sequence
  Synthetic polypeptide
QTIEERLTW

SEQ ID NO: 157 - protein, 10 aa, Artificial Sequence
  Synthetic polypeptide
VMANVLTLNL

SEQ ID NO: 158 - protein, 9 aa, Artificial Sequence
  Synthetic polypeptide
VLEDTLLKI

SEQ ID NO: 159 - protein, 9 aa, Artificial Sequence
  Synthetic polypeptide
KLYEAVPQL

SEQ ID NO: 160 - protein, 9 aa, Artificial Sequence
  Synthetic polypeptide
AGIGWGASY

SEQ ID NO: 161 - protein, 9 aa, Artificial Sequence
  Synthetic polypeptide
RMASTSCAA

SEQ ID NO: 162 - protein, 10 aa, Artificial Sequence
  Synthetic polypeptide
TPRKLVGRAV

SEQ ID NO: 163 - protein, 9 aa, Artificial Sequence
  Synthetic polypeptide
WLPKMPPFV

SEQ ID NO: 164 - protein, 9 aa, Artificial Sequence
  Synthetic polypeptide
SQNWGSLPL

SEQ ID NO: 165 - protein, 10 aa, Artificial Sequence
  Synthetic polypeptide
GLLHAVQEKL

SEQ ID NO: 166 - protein, 10 aa, Artificial Sequence
  Synthetic polypeptide
ATLVTPPTRY

SEQ ID NO: 167 - protein, 9 aa, Artificial Sequence
  Synthetic polypeptide
IAFSQLIGM

SEQ ID NO: 168 - protein, 9 aa, Artificial Sequence
  Synthetic polypeptide
LSNVAPPAF

SEQ ID NO: 169 - protein, 9 aa, Artificial Sequence
  Synthetic polypeptide
KVPFFSALK

SEQ ID NO: 170 - protein, 11 aa, Artificial Sequence
  Synthetic polypeptide
RFCPASCSGCY

SEQ ID NO: 171 - protein, 9 aa, Artificial Sequence
  Synthetic polypeptide
RTHPYSPKK

SEQ ID NO: 172 - protein, 10 aa, Artificial Sequence
  Synthetic polypeptide
VSNIAQAPLY

SEQ ID NO: 173 - protein, 9 aa, Artificial Sequence
  Synthetic polypeptide
YVAIRPLPY

SEQ ID NO: 174 - protein, 9 aa, Artificial Sequence
  Synthetic polypeptide
MYFFWPCSL

SEQ ID NO: 175 - protein, 8 aa, Artificial Sequence
  Synthetic polypeptide
ILFFFSSK

SEQ ID NO: 176 - protein, 9 aa, Artificial Sequence
  Synthetic polypeptide
HTCKVCVSF

SEQ ID NO: 177 - protein, 10 aa, Artificial Sequence
  Synthetic polypeptide
LAYWEKREAW

SEQ ID NO: 178 - protein, 9 aa, Artificial Sequence
  Synthetic polypeptide
HNVQGFHPY

SEQ ID NO: 179 - protein, 9 aa, Artificial Sequence
  Synthetic polypeptide
SMAASPSPK

SEQ ID NO: 180 - protein, 9 aa, Artificial Sequence
  Synthetic polypeptide
RAFSTFPSF

SEQ ID NO: 181 - protein, 9 aa, Artificial Sequence
  Synthetic polypeptide
KSVRGLELL

SEQ ID NO: 182 - protein, 9 aa, Artificial Sequence
  Synthetic polypeptide
QSSLSEKKF

SEQ ID NO: 183 - protein, 10 aa, Artificial Sequence
  Synthetic polypeptide
RSLMSVASAY

SEQ ID NO: 184 - protein, 10 aa, Artificial Sequence
  Synthetic polypeptide
GTNFWGVPRK

SEQ ID NO: 185 - protein, 11 aa, Artificial Sequence
  Synthetic polypeptide
LSYNLGAGEAL

SEQ ID NO: 186 - protein, 8 aa, Artificial Sequence
  Synthetic polypeptide
ITSPALLL

SEQ ID NO: 187 - protein, 9 aa, Artificial Sequence
  Synthetic polypeptide
SSNSCASAF

SEQ ID NO: 188 - protein, 11 aa, Artificial Sequence
  Synthetic polypeptide
GSYPSGSPCVW

SEQ ID NO: 189 - protein, 9 aa, Artificial Sequence
  Synthetic polypeptide
HSASNGTPL

SEQ ID NO: 190 - protein, 10 aa, Artificial Sequence
  Synthetic polypeptide
KLVGRAVRRK

SEQ ID NO: 191 - protein, 9 aa, Artificial Sequence
  Synthetic polypeptide
ILKEAPRRK

SEQ ID NO: 192 - protein, 9 aa, Artificial Sequence
  Synthetic polypeptide
STSFLDTRF

SEQ ID NO: 193 - protein, 10 aa, Artificial Sequence
  Synthetic polypeptide
KHTEKKSLSF

SEQ ID NO: 194 - protein, 9 aa, Artificial Sequence
  Synthetic polypeptide
FTHFHGEIW

SEQ ID NO: 195 - protein, 9 aa, Artificial Sequence
  Synthetic polypeptide
AVLGTMVMK

SEQ ID NO: 196 - protein, 9 aa, Artificial Sequence
  Synthetic polypeptide
QAGTPVMMF

SEQ ID NO: 197 - protein, 9 aa, Artificial Sequence
  Synthetic polypeptide
MTLFSLVPL

SEQ ID NO: 198 - protein, 10 aa, Artificial Sequence
  Synthetic polypeptide
SSLGTSDPRW

SEQ ID NO: 199 - protein, 9 aa, Artificial Sequence
  Synthetic polypeptide
HSLVQMEPL

SEQ ID NO: 200 - protein, 9 aa, Artificial Sequence
  Synthetic polypeptide
LTFCTNATI

SEQ ID NO: 201 - protein, 9 aa, Artificial Sequence
  Synthetic polypeptide
YLFAKAYLV

SEQ ID NO: 202 - protein, 9 aa, Artificial Sequence
  Synthetic polypeptide
VPACSHVPM

SEQ ID NO: 203 - protein, 9 aa, Artificial Sequence
  Synthetic polypeptide
VPTWSARLL

SEQ ID NO: 204 - protein, 9 aa, Artificial Sequence
  Synthetic polypeptide
LLNPVTMNK

SEQ ID NO: 205 - protein, 11 aa, Artificial Sequence
  Synthetic polypeptide
SELLNPVTMNK

SEQ ID NO: 206 - protein, 10 aa, Artificial Sequence
  Synthetic polypeptide
LLNPVTMNKA

SEQ ID NO: 207 - protein, 10 aa, Artificial Sequence
  Synthetic polypeptide
ELLNPVTMNK

SEQ ID NO: 208 - protein, 9 aa, Artificial Sequence
  Synthetic polypeptide
HPARHLCRL

SEQ ID NO: 209 - protein, 10 aa, Artificial Sequence
  Synthetic polypeptide
APSLTPMHSL

SEQ ID NO: 210 - protein, 8 aa, Artificial Sequence
  Synthetic polypeptide
SPMITRIL

SEQ ID NO: 211 - protein, 11 aa, Artificial Sequence
  Synthetic polypeptide
TPMHSLLISPM

SEQ ID NO: 212 - protein, 9 aa, Artificial Sequence
  Synthetic polypeptide
TPMHSLLIS

SEQ ID NO: 213 - protein, 10 aa, Artificial Sequence
  Synthetic polypeptide
RLYKYDHNFV

SEQ ID NO: 214 - protein, 10 aa, Artificial Sequence
  Synthetic polypeptide
RQMLPTLSTL

SEQ ID NO: 215 - protein, 10 aa, Artificial Sequence
  Synthetic polypeptide
RLDKGNFAGA

SEQ ID NO: 216 - protein, 10 aa, Artificial Sequence
  Synthetic polypeptide
MLLELSPAQL

SEQ ID NO: 217 - protein, 10 aa, Artificial Sequence
  Synthetic polypeptide
HPPPPCLLLL

SEQ ID NO: 218 - protein, 15 aa, Artificial Sequence
  Synthetic polypeptide
RWMVLRNSWRAVARM

SEQ ID NO: 219 - protein, 15 aa, Artificial Sequence
  Synthetic polypeptide
IDNIKRNHNLALGRQ

SEQ ID NO: 220 - protein, 15 aa, Artificial Sequence
  Synthetic polypeptide
PKMQVTITLTSPIIR

SEQ ID NO: 221 - protein, 15 aa, Artificial Sequence
  Synthetic polypeptide
FQVHFLKSGGLPLVL

SEQ ID NO: 222 - protein, 15 aa, Artificial Sequence
  Synthetic polypeptide
KTGLQLLRNHIEELK

SEQ ID NO: 223 - protein, 15 aa, Artificial Sequence
  Synthetic polypeptide
QKKLMLLRLNLRKMC

SEQ ID NO: 224 - protein, 15 aa, Artificial Sequence
  Synthetic polypeptide
LINIHHRKNPLLPMR

SEQ ID NO: 225 - protein, 15 aa, Artificial Sequence
  Synthetic polypeptide
WILHLLGLRPPSLLS

SEQ ID NO: 226 - protein, 15 aa, Artificial Sequence
  Synthetic polypeptide
LKETKFITYRSKKLI

SEQ ID NO: 227 - protein, 15 aa, Artificial Sequence
  Synthetic polypeptide
EDIEFHFSLGWTMLV

SEQ ID NO: 228 - protein, 15 aa, Artificial Sequence
  Synthetic polypeptide
GKNGFLQSRSSSLFS

SEQ ID NO: 229 - protein, 15 aa, Artificial Sequence
  Synthetic polypeptide
PALLLAEATHKASAL

SEQ ID NO: 230 - protein, 15 aa, Artificial Sequence
  Synthetic polypeptide
SLDNVLRTMLRRFAR

SEQ ID NO: 231 - protein, 15 aa, Artificial Sequence
  Synthetic polypeptide
YLRFIKSLAERTMSV

SEQ ID NO: 232 - protein, 15 aa, Artificial Sequence
  Synthetic polypeptide
KKDFGKMTANSVSVA

SEQ ID NO: 233 - protein, 15 aa, Artificial Sequence
  Synthetic polypeptide
RHVIKVLLGRKVNWH

SEQ ID NO: 234 - protein, 15 aa, Artificial Sequence
  Synthetic polypeptide
QLARFFPITPPVWHI

SEQ ID NO: 235 - protein, 15 aa, Artificial Sequence
  Synthetic polypeptide
NAILLFLRTRGVCSV

SEQ ID NO: 236 - protein, 15 aa, Artificial Sequence
  Synthetic polypeptide
FEEIIKNDGALLKKK

SEQ ID NO: 237 - protein, 15 aa, Artificial Sequence
  Synthetic polypeptide
LRLLSLYRPPLAPLL

SEQ ID NO: 238 - protein, 15 aa, Artificial Sequence
  Synthetic polypeptide
TKFITYRSKKLIQES

SEQ ID NO: 239 - protein, 15 aa, Artificial Sequence
  Synthetic polypeptide
LEVMLLNMGYRITGL

SEQ ID NO: 240 - protein, 15 aa, Artificial Sequence
  Synthetic polypeptide
SINVLCVRASLIEKL

SEQ ID NO: 241 - protein, 15 aa, Artificial Sequence
  Synthetic polypeptide
DDVLRNLKNFLLMKR

SEQ ID NO: 242 - protein, 15 aa, Artificial Sequence
  Synthetic polypeptide
LGKLEMVKAVQLRVA

SEQ ID NO: 243 - protein, 15 aa, Artificial Sequence
  Synthetic polypeptide
TARISVNSNNVQSLL

SEQ ID NO: 244 - protein, 15 aa, Artificial Sequence
  Synthetic polypeptide
SPMALLLAARQRAQK

SEQ ID NO: 245 - protein, 15 aa, Artificial Sequence
  Synthetic polypeptide
GKVIMPLGSKLTGVI

SEQ ID NO: 246 - protein, 15 aa, Artificial Sequence
  Synthetic polypeptide
KKVRVIYTQLSKTVV

SEQ ID NO: 247 - protein, 15 aa, Artificial Sequence
  Synthetic polypeptide
AFFMNLTREPSRVLK

SEQ ID NO: 248 - protein, 15 aa, Artificial Sequence
  Synthetic polypeptide
PSKRSLLSVGNLIGL

SEQ ID NO: 249 - protein, 15 aa, Artificial Sequence
  Synthetic polypeptide
PLPTALRQLRGRPAD

SEQ ID NO: 250 - protein, 15 aa, Artificial Sequence
  Synthetic polypeptide
TVEMRRWWTLVMEWK

SEQ ID NO: 251 - protein, 15 aa, Artificial Sequence
  Synthetic polypeptide
NKKMLTALPPAMTAM

SEQ ID NO: 252 - protein, 15 aa, Artificial Sequence
  Synthetic polypeptide
KKKRQINRRKLQRKK

SEQ ID NO: 253 - protein, 15 aa, Artificial Sequence
  Synthetic polypeptide
LKMVWRINPAHRKLQ

SEQ ID NO: 254 - protein, 15 aa, Artificial Sequence
  Synthetic polypeptide
PHRLRSLPRPLHLRL

SEQ ID NO: 255 - protein, 15 aa, Artificial Sequence
  Synthetic polypeptide
NQNLYLVGASKIRML

SEQ ID NO: 256 - protein, 15 aa, Artificial Sequence
  Synthetic polypeptide
FHPYRRYPPPAAAAL

SEQ ID NO: 257 - protein, 15 aa, Artificial Sequence
  Synthetic polypeptide
ESNLLQSPSSILSTL

SEQ ID NO: 258 - protein, 15 aa, Artificial Sequence
  Synthetic polypeptide
LWGVRMTSLSASTSL

SEQ ID NO: 259 - protein, 15 aa, Artificial Sequence
  Synthetic polypeptide
LSSLVKKILAMTLTL

SEQ ID NO: 260 - protein, 15 aa, Artificial Sequence
  Synthetic polypeptide
KKTVLSLVTISRFVL

SEQ ID NO: 261 - protein, 15 aa, Artificial Sequence
  Synthetic polypeptide
NSVIVGNTHGQLAEI

SEQ ID NO: 262 - protein, 15 aa, Artificial Sequence
  Synthetic polypeptide
QLPVYKLLPSQNRLQ

SEQ ID NO: 263 - protein, 15 aa, Artificial Sequence
  Synthetic polypeptide
IRKGFQLRKTARGRG

SEQ ID NO: 264 - protein, 15 aa, Artificial Sequence
  Synthetic polypeptide
GVFISKVLPRGLAAR

SEQ ID NO: 265 - protein, 15 aa, Artificial Sequence
  Synthetic polypeptide
QRRLIKSMESVMVKY

SEQ ID NO: 266 - protein, 15 aa, Artificial Sequence
  Synthetic polypeptide
KKNILNSLPSSMEIA

SEQ ID NO: 267 - protein, 15 aa, Artificial Sequence
  Synthetic polypeptide
NRVFKLAPNLTELRA

SEQ ID NO: 268 - protein, 15 aa, Artificial Sequence
  Synthetic polypeptide
KLICQMTRTNRLFGM

SEQ ID NO: 269 - protein, 15 aa, Artificial Sequence
  Synthetic polypeptide
NSKLRYKKRGVIAWR

SEQ ID NO: 270 - protein, 15 aa, Artificial Sequence
  Synthetic polypeptide
SFASMGMLEARIRIL

SEQ ID NO: 271 - protein, 15 aa, Artificial Sequence
  Synthetic polypeptide
DARLRASTALLLPIL

SEQ ID NO: 272 - protein, 15 aa, Artificial Sequence
  Synthetic polypeptide
ARLCLIVSRTLLLVQ

SEQ ID NO: 273 - protein, 15 aa, Artificial Sequence
  Synthetic polypeptide
ALSVLTASLSYMVGM

SEQ ID NO: 274 - protein, 15 aa, Artificial Sequence
  Synthetic polypeptide
AWFIRESMTIYIFCL

SEQ ID NO: 275 - protein, 15 aa, Artificial Sequence
  Synthetic polypeptide
ERLLFFAVPPQILAS

SEQ ID NO: 276 - protein, 15 aa, Artificial Sequence
  Synthetic polypeptide
MFFMVFLIIWQNTMF

SEQ ID NO: 277 - protein, 15 aa, Artificial Sequence
  Synthetic polypeptide
VFFAYLVAHSFLSVF

SEQ ID NO: 278 - protein, 15 aa, Artificial Sequence
  Synthetic polypeptide
GTGASMASIMETIGL

SEQ ID NO: 279 - protein, 15 aa, Artificial Sequence
  Synthetic polypeptide
LLPYPFHVLALEVTF

SEQ ID NO: 280 - protein, 15 aa, Artificial Sequence
  Synthetic polypeptide
PLRICVTLWSRLVLA

SEQ ID NO: 281 - protein, 15 aa, Artificial Sequence
  Synthetic polypeptide
ERVQTVAASTMRVAV

SEQ ID NO: 282 - protein, 15 aa, Artificial Sequence
  Synthetic polypeptide
YQYTVFLRSDSYMGL

SEQ ID NO: 283 - protein, 15 aa, Artificial Sequence
  Synthetic polypeptide
QRYRSVLRGWWILLT

SEQ ID NO: 284 - protein, 15 aa, Artificial Sequence
  Synthetic polypeptide
LYGWYQLCVSSMKLL

SEQ ID NO: 285 - protein, 15 aa, Artificial Sequence
  Synthetic polypeptide
TGATCGKRAARLVLR

SEQ ID NO: 286 - protein, 15 aa, Artificial Sequence
  Synthetic polypeptide
LSDIYLNNVIMRFMQ

SEQ ID NO: 287 - protein, 15 aa, Artificial Sequence
  Synthetic polypeptide
RLFLLQDSGRILQLL

SEQ ID NO: 288 - protein, 15 aa, Artificial Sequence
  Synthetic polypeptide
APQVTRLRSLNHLLI

SEQ ID NO: 289 - protein, 15 aa, Artificial Sequence
  Synthetic polypeptide
GIFLVIETHGMAVSW

SEQ ID NO: 290 - protein, 15 aa, Artificial Sequence
  Synthetic polypeptide
ANRYFSPNFKVKLYF

SEQ ID NO: 291 - protein, 15 aa, Artificial Sequence
  Synthetic polypeptide
WRLFLIIQTTGYQSI

SEQ ID NO: 292 - protein, 15 aa, Artificial Sequence
  Synthetic polypeptide
GSQSIMMSWMPPLAP

SEQ ID NO: 293 - protein, 15 aa, Artificial Sequence
  Synthetic polypeptide
GNLLSFSRRGMKSSV

SEQ ID NO: 294 - protein, 15 aa, Artificial Sequence
  Synthetic polypeptide
HLTLARMKHFIYFKH

SEQ ID NO: 295 - protein, 15 aa, Artificial Sequence
  Synthetic polypeptide
YTIFYRTIIGNETAV

SEQ ID NO: 296 - protein, 15 aa, Artificial Sequence
  Synthetic polypeptide
WKVKLPSSMSVALPL

SEQ ID NO: 297 - protein, 15 aa, Artificial Sequence
  Synthetic polypeptide
FQELILNQASMAPPR

SEQ ID NO: 298 - protein, 15 aa, Artificial Sequence
  Synthetic polypeptide
KERLFRNFGGLLGPL

SEQ ID NO: 299 - protein, 15 aa, Artificial Sequence
  Synthetic polypeptide
YSKVRALGGVNAARR

SEQ ID NO: 300 - protein, 15 aa, Artificial Sequence
  Synthetic polypeptide
MMIVLTIQNAAFLSN

SEQ ID NO: 301 - protein, 15 aa, Artificial Sequence
  Synthetic polypeptide
LIWIVFISSGHVASA

SEQ ID NO: 302 - protein, 15 aa, Artificial Sequence
  Synthetic polypeptide
EPFIQKDVELRIMPP

SEQ ID NO: 303 - protein, 15 aa, Artificial Sequence
  Synthetic polypeptide
GRLQIMSLENLSIEK

SEQ ID NO: 304 - protein, 15 aa, Artificial Sequence
  Synthetic polypeptide
WPITELKIQMRGILG

SEQ ID NO: 305 - protein, 15 aa, Artificial Sequence
  Synthetic polypeptide
RELLKTLNMIQVLMS

SEQ ID NO: 306 - protein, 15 aa, Artificial Sequence
  Synthetic polypeptide
SAFSSLLPLRNLSQL

SEQ ID NO: 307 - protein, 15 aa, Artificial Sequence
  Synthetic polypeptide
ILGSFFMATSSHRFL

SEQ ID NO: 308 - protein, 15 aa, Artificial Sequence
  Synthetic polypeptide
RSRLMRQSRRSTQGV

SEQ ID NO: 309 - protein, 15 aa, Artificial Sequence
  Synthetic polypeptide
PSWPMAVPLAASRAS

SEQ ID NO: 310 - protein, 15 aa, Artificial Sequence
  Synthetic polypeptide
KLQAVLEDTLLKILL

SEQ ID NO: 311 - protein, 15 aa, Artificial Sequence
  Synthetic polypeptide
KSLVRLSSCVPVALM

SEQ ID NO: 312 - protein, 15 aa, Artificial Sequence
  Synthetic polypeptide
ACRLRWARPEPAAQA

SEQ ID NO: 313 - protein, 15 aa, Artificial Sequence
  Synthetic polypeptide
IKICILWNQIMHASW

SEQ ID NO: 314 - protein, 15 aa, Artificial Sequence
  Synthetic polypeptide
HVPLSISGSPALELL

SEQ ID NO: 315 - protein, 15 aa, Artificial Sequence
  Synthetic polypeptide
HKKLILEKSPINVKK

SEQ ID NO: 316 - protein, 15 aa, Artificial Sequence
  Synthetic polypeptide
EKDLMQLAQATAVAA

SEQ ID NO: 317 - protein, 15 aa, Artificial Sequence
  Synthetic polypeptide
FTIYSISSLKTLFRK

SEQ ID NO: 318 - protein, 15 aa, Artificial Sequence
  Synthetic polypeptide
LRGGVIQSTRRRRRA

SEQ ID NO: 319 - protein, 15 aa, Artificial Sequence
  Synthetic polypeptide
PCASLLSTLSQPPPQ

SEQ ID NO: 320 - protein, 15 aa, Artificial Sequence
  Synthetic polypeptide
EDMQEVVVHKKRGLF

SEQ ID NO: 321 - protein, 15 aa, Artificial Sequence
  Synthetic polypeptide
RFPLLMMWRTPMTTR

SEQ ID NO: 322 - protein, 15 aa, Artificial Sequence
  Synthetic polypeptide
PVLFKADQSESSLSS

SEQ ID NO: 323 - protein, 15 aa, Artificial Sequence
  Synthetic polypeptide
RIPAVLRTEGEPLHT

SEQ ID NO: 324 - protein, 15 aa, Artificial Sequence
  Synthetic polypeptide
FGILNVLEFSSDRKK

SEQ ID NO: 325 - protein, 15 aa, Artificial Sequence
  Synthetic polypeptide
ILVALSWMGGLHSFY

SEQ ID NO: 326 - protein, 15 aa, Artificial Sequence
  Synthetic polypeptide
LGQIMASAVEASQPP

SEQ ID NO: 327 - protein, 15 aa, Artificial Sequence
  Synthetic polypeptide
QRWLTSTTSRSSALM

SEQ ID NO: 328 - protein, 15 aa, Artificial Sequence
  Synthetic polypeptide
LLHWRIGGGTPLSIS

SEQ ID NO: 329 - protein, 15 aa, Artificial Sequence
  Synthetic polypeptide
EIQLTMNDSKHKLES

SEQ ID NO: 330 - protein, 15 aa, Artificial Sequence
  Synthetic polypeptide
LTCALHNDGIYIMSR

SEQ ID NO: 331 - protein, 15 aa, Artificial Sequence
  Synthetic polypeptide
CILVALSWRGASFIL

SEQ ID NO: 332 - protein, 15 aa, Artificial Sequence
  Synthetic polypeptide
GVRILKLCSKVSFRV

SEQ ID NO: 333 - protein, 15 aa, Artificial Sequence
  Synthetic polypeptide
LRAVVVDDYRRRKKR

SEQ ID NO: 334 - protein, 15 aa, Artificial Sequence
  Synthetic polypeptide
ALGLRILPPPLTSPS

SEQ ID NO: 335 - protein, 15 aa, Artificial Sequence
  Synthetic polypeptide
KKSVLKAIEQADLLQ

SEQ ID NO: 336 - protein, 15 aa, Artificial Sequence
  Synthetic polypeptide
KEALFLQEVFQAERL

SEQ ID NO: 337 - protein, 15 aa, Artificial Sequence
  Synthetic polypeptide
WTPRKLVGRAVRRKG

SEQ ID NO: 338 - protein, 15 aa, Artificial Sequence
  Synthetic polypeptide
ADRAFMAAQKCHKKT

SEQ ID NO: 339 - protein, 15 aa, Artificial Sequence
  Synthetic polypeptide
FGYATSISMAQASDG

SEQ ID NO: 340 - protein, 15 aa, Artificial Sequence
  Synthetic polypeptide
KYRCFSYLPISPTFV

SEQ ID NO: 341 - protein, 15 aa, Artificial Sequence
  Synthetic polypeptide
LRGELSYNLGAGEAL

SEQ ID NO: 342 - protein, 15 aa, Artificial Sequence
  Synthetic polypeptide
ARHRLASFKTVIKKR

SEQ ID NO: 343 - protein, 15 aa, Artificial Sequence
  Synthetic polypeptide
LRELRLDNSVAIHYI

SEQ ID NO: 344 - protein, 15 aa, Artificial Sequence
  Synthetic polypeptide
AAPEIILGNPVSLTS

SEQ ID NO: 345 - protein, 15 aa, Artificial Sequence
  Synthetic polypeptide
RLSLVQSSSWPTVLH

SEQ ID NO: 346 - protein, 15 aa, Artificial Sequence
  Synthetic polypeptide
SRQPSPLLLLPPLPA

SEQ ID NO: 347 - protein, 15 aa, Artificial Sequence
  Synthetic polypeptide
KRLMSLSPGRPPLLL

SEQ ID NO: 348 - protein, 15 aa, Artificial Sequence
  Synthetic polypeptide
KSLLFPSAPASVMNA

SEQ ID NO: 349 - protein, 15 aa, Artificial Sequence
  Synthetic polypeptide
QGKLQQHHVLRVSRR

SEQ ID NO: 350 - protein, 15 aa, Artificial Sequence
  Synthetic polypeptide
AEIRAQDAPLSLLQT

SEQ ID NO: 351 - protein, 15 aa, Artificial Sequence
  Synthetic polypeptide
HGSHNIKKAWYLIAM

SEQ ID NO: 352 - protein, 15 aa, Artificial Sequence
  Synthetic polypeptide
RCCLRTSCGAARPRR

SEQ ID NO: 353 - protein, 15 aa, Artificial Sequence
  Synthetic polypeptide
LCNRLLKSFSKWSLV

SEQ ID NO: 354 - protein, 15 aa, Artificial Sequence
  Synthetic polypeptide
PVTPLRVQSVLLLGV

SEQ ID NO: 355 - protein, 15 aa, Artificial Sequence
  Synthetic polypeptide
IAGYRESAAFLLRSA

SEQ ID NO: 356 - protein, 15 aa, Artificial Sequence
  Synthetic polypeptide
SGTARLARTAIAAST

SEQ ID NO: 357 - protein, 15 aa, Artificial Sequence
  Synthetic polypeptide
NQPVEFNHAINYLIR

SEQ ID NO: 358 - protein, 15 aa, Artificial Sequence
  Synthetic polypeptide
RRPLRSWTPRTRGAH

SEQ ID NO: 359 - protein, 15 aa, Artificial Sequence
  Synthetic polypeptide
NEIQKLQKTLKKKPR

SEQ ID NO: 360 - protein, 15 aa, Artificial Sequence
  Synthetic polypeptide
PRLVKMISSISLEIW

SEQ ID NO: 361 - protein, 15 aa, Artificial Sequence
  Synthetic polypeptide
SSIFIGGSFILKKKA

SEQ ID NO: 362 - protein, 15 aa, Artificial Sequence
  Synthetic polypeptide
PALLLPATTCKVPRL

SEQ ID NO: 363 - protein, 15 aa, Artificial Sequence
  Synthetic polypeptide
KKRKKFMKDAKKRGR

SEQ ID NO: 364 - protein, 15 aa, Artificial Sequence
  Synthetic polypeptide
REYMLNLFKALKRIH

SEQ ID NO: 365 - protein, 15 aa, Artificial Sequence
  Synthetic polypeptide
APQGFRATLVPPALE

SEQ ID NO: 366 - protein, 15 aa, Artificial Sequence
  Synthetic polypeptide
TTVGLLRMAATRTSL

SEQ ID NO: 367 - protein, 15 aa, Artificial Sequence
  Synthetic polypeptide
DTWAALEGLSSPFRE

SEQ ID NO: 368 - protein, 15 aa, Artificial Sequence
  Synthetic polypeptide
KQGWLHKKGGGLLHA

SEQ ID NO: 369 - protein, 15 aa, Artificial Sequence
  Synthetic polypeptide
LTETVYSTTQQIHSS

SEQ ID NO: 370 - protein, 15 aa, Artificial Sequence
  Synthetic polypeptide
NPAFDVTPTTSSLVA

SEQ ID NO: 371 - protein, 15 aa, Artificial Sequence
  Synthetic polypeptide
ETNMGIIAGVAFGIA

SEQ ID NO: 372 - protein, 15 aa, Artificial Sequence
  Synthetic polypeptide
SSFFCRCRREYRVTM

SEQ ID NO: 373 - protein, 15 aa, Artificial Sequence
  Synthetic polypeptide
PGCFWPCLWNSLLRF

SEQ ID NO: 374 - protein, 15 aa, Artificial Sequence
  Synthetic polypeptide
FPILFFFSSKGVRAT

SEQ ID NO: 375 - protein, 15 aa, Artificial Sequence
  Synthetic polypeptide
IAYYIEGIENSVFFF

SEQ ID NO: 376 - protein, 15 aa, Artificial Sequence
  Synthetic polypeptide
YDCYVAIRPLPYATR

SEQ ID NO: 377 - protein, 15 aa, Artificial Sequence
  Synthetic polypeptide
DRINANTITSPALLL

SEQ ID NO: 378 - protein, 15 aa, Artificial Sequence
  Synthetic polypeptide
HAFIVPIRSLQDHTH

SEQ ID NO: 379 - protein, 15 aa, Artificial Sequence
  Synthetic polypeptide
PKKKRSAFPSRSLSS

SEQ ID NO: 380 - protein, 15 aa, Artificial Sequence
  Synthetic polypeptide
ILLGPLLPNVVFYIL

SEQ ID NO: 381 - protein, 15 aa, Artificial Sequence
  Synthetic polypeptide
PPGVVLNNISSYASV

SEQ ID NO: 382 - protein, 15 aa, Artificial Sequence
  Synthetic polypeptide
WEAAMMNGKVPFFSA

SEQ ID NO: 383 - protein, 15 aa, Artificial Sequence
  Synthetic polypeptide
SLQSAVSNIAQAPLY

SEQ ID NO: 384 - protein, 15 aa, Artificial Sequence
  Synthetic polypeptide
RPQVRLAGAQAIFEA

SEQ ID NO: 385 - protein, 15 aa, Artificial Sequence
  Synthetic polypeptide
PRFSQIHSILSKMVQ

SEQ ID NO: 386 - protein, 15 aa, Artificial Sequence
  Synthetic polypeptide
KETLRLNPPVPGGFR

SEQ ID NO: 387 - protein, 15 aa, Artificial Sequence
  Synthetic polypeptide
SRRSLMSVASAYSAK

SEQ ID NO: 388 - protein, 15 aa, Artificial Sequence
  Synthetic polypeptide
REAGRFAVLGTMVMK

SEQ ID NO: 389 - protein, 15 aa, Artificial Sequence
  Synthetic polypeptide
HSTPFQAGTPVMMFL

SEQ ID NO: 390 - protein, 15 aa, Artificial Sequence
  Synthetic polypeptide
RLMPKFLNSTNSWWT

SEQ ID NO: 391 - protein, 15 aa, Artificial Sequence
  Synthetic polypeptide
LATAAAAAAAAAFGD

SEQ ID NO: 392 - protein, 15 aa, Artificial Sequence
  Synthetic polypeptide
KRLSKVETLRAAIDY

SEQ ID NO: 393 - protein, 15 aa, Artificial Sequence
  Synthetic polypeptide
LAQEQKKKKSWRLLL

SEQ ID NO: 394 - protein, 15 aa, Artificial Sequence
  Synthetic polypeptide
ITRQQWKKALRSMPK

SEQ ID NO: 395 - protein, 15 aa, Artificial Sequence
  Synthetic polypeptide
SRPLAHSVASTLAPA

SEQ ID NO: 396 - protein, 15 aa, Artificial Sequence
  Synthetic polypeptide
IGSISRQSSLSEKKF

SEQ ID NO: 397 - protein, 15 aa, Artificial Sequence
  Synthetic polypeptide
GLCILKEAPRRKRPA

SEQ ID NO: 398 - protein, 15 aa, Artificial Sequence
  Synthetic polypeptide
RKMILSQLHHSMTRF

SEQ ID NO: 399 - protein, 15 aa, Artificial Sequence
  Synthetic polypeptide
ILQHFLLHATPQTQL

SEQ ID NO: 400 - protein, 15 aa, Artificial Sequence
  Synthetic polypeptide
QAHLPSAPALPPPTH

SEQ ID NO: 401 - protein, 15 aa, Artificial Sequence
  Synthetic polypeptide
GHSLVQMEPLTTARP

SEQ ID NO: 402 - protein, 15 aa, Artificial Sequence
  Synthetic polypeptide
GVHSTIKVIKAKKKH

SEQ ID NO: 403 - protein, 15 aa, Artificial Sequence
  Synthetic polypeptide
QGGLLMGYSPAGGRH

SEQ ID NO: 404 - protein, 15 aa, Artificial Sequence
  Synthetic polypeptide
LLRMTLFSLVPLEPI

SEQ ID NO: 405 - protein, 10 aa, Artificial Sequence
  Synthetic polypeptide
KSAFATYKVK

SEQ ID NO: 406 - protein, 9 aa, Artificial Sequence
  Synthetic polypeptide
NLHLKTTSL

SEQ ID NO: 407 - protein, 10 aa, Artificial Sequence
  Synthetic polypeptide
FLRQRATSTI

SEQ ID NO: 408 - protein, 9 aa, Artificial Sequence
  Synthetic polypeptide
LIKLKLNRL

SEQ ID NO: 409 - protein, 8 aa, Artificial Sequence
  Synthetic polypeptide
LMLLRLNL

SEQ ID NO: 410 - protein, 9 aa, Artificial Sequence
  Synthetic polypeptide
HFINVMFVR

SEQ ID NO: 411 - protein, 9 aa, Artificial Sequence
  Synthetic polypeptide
SALVRLFPV

SEQ ID NO: 412 - protein, 9 aa, Artificial Sequence
  Synthetic polypeptide
FPKKKCTNL

SEQ ID NO: 413 - protein, 9 aa, Artificial Sequence
  Synthetic polypeptide
MSVKKVMTY

SEQ ID NO: 414 - protein, 10 aa, Artificial Sequence
  Synthetic polypeptide
FATYKAKMPL

SEQ ID NO: 415 - protein, 9 aa, Artificial Sequence
  Synthetic polypeptide
MTLSKMIKK

SEQ ID NO: 416 - protein, 9 aa, Artificial Sequence
  Synthetic polypeptide
NPRRKTWKM

SEQ ID NO: 417 - protein, 9 aa, Artificial Sequence
  Synthetic polypeptide
SQKKRRYSI

SEQ ID NO: 418 - protein, 9 aa, Artificial Sequence
  Synthetic polypeptide
KTTNHSSQM

SEQ ID NO: 419 - protein, 9 aa, Artificial Sequence
  Synthetic polypeptide
AAICTTPAL

SEQ ID NO: 420 - protein, 9 aa, Artificial Sequence
  Synthetic polypeptide
TATETKTPY

SEQ ID NO: 421 - protein, 9 aa, Artificial Sequence
  Synthetic polypeptide
RIKKKLMEL

SEQ ID NO: 422 - protein, 9 aa, Artificial Sequence
  Synthetic polypeptide
HSYHLLQAY

SEQ ID NO: 423 - protein, 9 aa, Artificial Sequence
  Synthetic polypeptide
LSSVSFFLY

SEQ ID NO: 424 - protein, 9 aa, Artificial Sequence
  Synthetic polypeptide
KRATFLLAL

SEQ ID NO: 425 - protein, 11 aa, Artificial Sequence
  Synthetic polypeptide
YYYGGNCGLFY

SEQ ID NO: 426 - protein, 9 aa, Artificial Sequence
  Synthetic polypeptide
LTMKEAVPK

SEQ ID NO: 427 - protein, 10 aa, Artificial Sequence
  Synthetic polypeptide
AEISSQVPHW

SEQ ID NO: 428 - protein, 9 aa, Artificial Sequence
  Synthetic polypeptide
AEKCLILVW

SEQ ID NO: 429 - protein, 9 aa, Artificial Sequence
  Synthetic polypeptide
TELGILTSF

SEQ ID NO: 430 - protein, 9 aa, Artificial Sequence
  Synthetic polypeptide
SLRRKYLRV

SEQ ID NO: 431 - protein, 10 aa, Artificial Sequence
  Synthetic polypeptide
SQFLTEGIMK

SEQ ID NO: 432 - protein, 10 aa, Artificial Sequence
  Synthetic polypeptide
LPLLRHHLPL

SEQ ID NO: 433 - protein, 9 aa, Artificial Sequence
  Synthetic polypeptide
FSDSEGEGL

SEQ ID NO: 434 - protein, 9 aa, Artificial Sequence
  Synthetic polypeptide
ITIGVRPIR

SEQ ID NO: 435 - protein, 8 aa, Artificial Sequence
  Synthetic polypeptide
YADQWTVL

SEQ ID NO: 436 - protein, 10 aa, Artificial Sequence
  Synthetic polypeptide
HTMWHCALEK

SEQ ID NO: 437 - protein, 8 aa, Artificial Sequence
  Synthetic polypeptide
SAKKRASV

SEQ ID NO: 438 - protein, 10 aa, Artificial Sequence
  Synthetic polypeptide
VVQKVAWFYK

SEQ ID NO: 439 - protein, 9 aa, Artificial Sequence
  Synthetic polypeptide
HPSGGPIPL

SEQ ID NO: 440 - protein, 10 aa, Artificial Sequence
  Synthetic polypeptide
KTVDHKVERK

SEQ ID NO: 441 - protein, 10 aa, Artificial Sequence
  Synthetic polypeptide
TYTICVTMPY

SEQ ID NO: 442 - protein, 9 aa, Artificial Sequence
  Synthetic polypeptide
TLRKKKQTV

SEQ ID NO: 443 - protein, 10 aa, Artificial Sequence
  Synthetic polypeptide
EEKMYYLFVY

SEQ ID NO: 444 - protein, 9 aa, Artificial Sequence
  Synthetic polypeptide
AENEKKILY

SEQ ID NO: 445 - protein, 9 aa, Artificial Sequence
  Synthetic polypeptide
HSPTPTSAL

SEQ ID NO: 446 - protein, 10 aa, Artificial Sequence
  Synthetic polypeptide
AEQEQEVVAW

SEQ ID NO: 447 - protein, 10 aa, Artificial Sequence
  Synthetic polypeptide
WPIQRCACSV

SEQ ID NO: 448 - protein, 9 aa, Artificial Sequence
  Synthetic polypeptide
HQKRRKKNL

SEQ ID NO: 449 - protein, 9 aa, Artificial Sequence
  Synthetic polypeptide
TLKTLFHLR

SEQ ID NO: 450 - protein, 10 aa, Artificial Sequence
  Synthetic polypeptide
KSFTKNHSSK

SEQ ID NO: 451 - protein, 9 aa, Artificial Sequence
  Synthetic polypeptide
MIKNRISPL

SEQ ID NO: 452 - protein, 10 aa, Artificial Sequence
  Synthetic polypeptide
NIKKKPGTSL

SEQ ID NO: 453 - protein, 10 aa, Artificial Sequence
  Synthetic polypeptide
AESLRENFSW

SEQ ID NO: 454 - protein, 9 aa, Artificial Sequence
  Synthetic polypeptide
TSVGLAWRW

SEQ ID NO: 455 - protein, 9 aa, Artificial Sequence
  Synthetic polypeptide
CLSLRKKAL

SEQ ID NO: 456 - protein, 11 aa, Artificial Sequence
  Synthetic polypeptide
VIIKKNPALPK

SEQ ID NO: 457 - protein, 9 aa, Artificial Sequence
  Synthetic polypeptide
GALPRIHNM

SEQ ID NO: 458 - protein, 10 aa, Artificial Sequence
  Synthetic polypeptide
AESGPFRPGW

SEQ ID NO: 459 - protein, 9 aa, Artificial Sequence
  Synthetic polypeptide
TSLARPPPL

SEQ ID NO: 460 - protein, 10 aa, Artificial Sequence
  Synthetic polypeptide
TPESPPLQLW

SEQ ID NO: 461 - protein, 9 aa, Artificial Sequence
  Synthetic polypeptide
KALADPSAF

SEQ ID NO: 462 - protein, 9 aa, Artificial Sequence
  Synthetic polypeptide
KRKSILLHL

SEQ ID NO: 463 - protein, 9 aa, Artificial Sequence
  Synthetic polypeptide
YSTEKRKKY

SEQ ID NO: 464 - protein, 9 aa, Artificial Sequence
  Synthetic polypeptide
RRRPRLPTL

SEQ ID NO: 465 - protein, 9 aa, Artificial Sequence
  Synthetic polypeptide
AMQDFFSYY

SEQ ID NO: 466 - protein, 10 aa, Artificial Sequence
  Synthetic polypeptide
TEVTWWALRK

SEQ ID NO: 467 - protein, 10 aa, Artificial Sequence
  Synthetic polypeptide
CTACHTALGR

SEQ ID NO: 468 - protein, 9 aa, Artificial Sequence
  Synthetic polypeptide
IEFRIKFLF

SEQ ID NO: 469 - protein, 9 aa, Artificial Sequence
  Synthetic polypeptide
ITMQQIAVL

SEQ ID NO: 470 - protein, 9 aa, Artificial Sequence
  Synthetic polypeptide
CLRPHRVQL

SEQ ID NO: 471 - protein, 9 aa, Artificial Sequence
  Synthetic polypeptide
KTHPIRTSL

SEQ ID NO: 472 - protein, 9 aa, Artificial Sequence
  Synthetic polypeptide
HAVGCPVQM

SEQ ID NO: 473 - protein, 9 aa, Artificial Sequence
  Synthetic polypeptide
TALVPPPAL

SEQ ID NO: 474 - protein, 9 aa, Artificial Sequence
  Synthetic polypeptide
WIIPFWFPF

SEQ ID NO: 475 - protein, 9 aa, Artificial Sequence
  Synthetic polypeptide
FTVITYFLW

SEQ ID NO: 476 - protein, 9 aa, Artificial Sequence
  Synthetic polypeptide
LFLVREVQR

SEQ ID NO: 477 - protein, 9 aa, Artificial Sequence
  Synthetic polypeptide
SSPPFHYPF

SEQ ID NO: 478 - protein, 9 aa, Artificial Sequence
  Synthetic polypeptide
RLHCQHSSL

SEQ ID NO: 479 - protein, 9 aa, Artificial Sequence
  Synthetic polypeptide
NSKKTNATF

SEQ ID NO: 480 - protein, 9 aa, Artificial Sequence
  Synthetic polypeptide
TKKRKMQSL

SEQ ID NO: 481 - protein, 9 aa, Artificial Sequence
  Synthetic polypeptide
DLVPSCHPR

SEQ ID NO: 482 - protein, 10 aa, Artificial Sequence
  Synthetic polypeptide
KMQFRLLVLL

SEQ ID NO: 483 - protein, 9 aa, Artificial Sequence
  Synthetic polypeptide
TLKRRTLAM

SEQ ID NO: 484 - protein, 9 aa, Artificial Sequence
  Synthetic polypeptide
TPALREYTM

SEQ ID NO: 485 - protein, 10 aa, Artificial Sequence
  Synthetic polypeptide
ATGLNIWKLK

SEQ ID NO: 486 - protein, 9 aa, Artificial Sequence
  Synthetic polypeptide
TTTCPSRPL

SEQ ID NO: 487 - protein, 9 aa, Artificial Sequence
  Synthetic polypeptide
FGMMSMASR

SEQ ID NO: 488 - protein, 9 aa, Artificial Sequence
  Synthetic polypeptide
FALCGFWQI

SEQ ID NO: 489 - protein, 10 aa, Artificial Sequence
  Synthetic polypeptide
QVYSVPHFFF

SEQ ID NO: 490 - protein, 10 aa, Artificial Sequence
  Synthetic polypeptide
SAASYLWPSR

SEQ ID NO: 491 - protein, 9 aa, Artificial Sequence
  Synthetic polypeptide
QLKKKHLKA

SEQ ID NO: 492 - protein, 9 aa, Artificial Sequence
  Synthetic polypeptide
GAKKHFGSF

SEQ ID NO: 493 - protein, 10 aa, Artificial Sequence
  Synthetic polypeptide
FPRSQHQSPL

SEQ ID NO: 494 - protein, 9 aa, Artificial Sequence
  Synthetic polypeptide
STAAPPAPR

SEQ ID NO: 495 - protein, 9 aa, Artificial Sequence
  Synthetic polypeptide
YLFEGAQTV

SEQ ID NO: 496 - protein, 11 aa, Artificial Sequence
  Synthetic polypeptide
TVLRVDQIMAK

SEQ ID NO: 497 - protein, 10 aa, Artificial Sequence
  Synthetic polypeptide
ELSICIRIPR

SEQ ID NO: 498 - protein, 11 aa, Artificial Sequence
  Synthetic polypeptide
KTYTCAITTVK

SEQ ID NO: 499 - protein, 10 aa, Artificial Sequence
  Synthetic polypeptide
WLEKKNCYSL

SEQ ID NO: 500 - protein, 9 aa, Artificial Sequence
  Synthetic polypeptide
RSVLWERVV

SEQ ID NO: 501 - protein, 9 aa, Artificial Sequence
  Synthetic polypeptide
HLLQEYLPL

SEQ ID NO: 502 - protein, 9 aa, Artificial Sequence
  Synthetic polypeptide
CPSPGPPSL

SEQ ID NO: 503 - protein, 10 aa, Artificial Sequence
  Synthetic polypeptide
MSVCFFFFCY

SEQ ID NO: 504 - protein, 10 aa, Artificial Sequence
  Synthetic polypeptide
VMSDTTYKIY

SEQ ID NO: 505 - protein, 9 aa, Artificial Sequence
  Synthetic polypeptide
QAHPQVPAL

SEQ ID NO: 506 - protein, 11 aa, Artificial Sequence
  Synthetic polypeptide
FPITPPVWHIL

SEQ ID NO: 507 - protein, 8 aa, Artificial Sequence
  Synthetic polypeptide
VVHKKRGL

SEQ ID NO: 508 - protein, 9 aa, Artificial Sequence
  Synthetic polypeptide
SRYPNICWF

SEQ ID NO: 509 - protein, 9 aa, Artificial Sequence
  Synthetic polypeptide
MTTISRATW

SEQ ID NO: 510 - protein, 9 aa, Artificial Sequence
  Synthetic polypeptide
SSWATCWPR

SEQ ID NO: 511 - protein, 10 aa, Artificial Sequence
  Synthetic polypeptide
RVRAHPGLPR

SEQ ID NO: 512 - protein, 11 aa, Artificial Sequence
  Synthetic polypeptide
STAHIPPLHLR

SEQ ID NO: 513 - protein, 9 aa, Artificial Sequence
  Synthetic polypeptide
RLWNKTRCR

SEQ ID NO: 514 - protein, 9 aa, Artificial Sequence
  Synthetic polypeptide
SSFLVSSSI

SEQ ID NO: 515 - protein, 11 aa, Artificial Sequence
  Synthetic polypeptide
TFMPPPGHPPR

SEQ ID NO: 516 - protein, 9 aa, Artificial Sequence
  Synthetic polypeptide
KTNATFSLV

SEQ ID NO: 517 - protein, 9 aa, Artificial Sequence
  Synthetic polypeptide
AQTRDRWRK

SEQ ID NO: 518 - protein, 10 aa, Artificial Sequence
  Synthetic polypeptide
STEVEETQEK

SEQ ID NO: 519 - protein, 9 aa, Artificial Sequence
  Synthetic polypeptide
TFKKKTYTC

SEQ ID NO: 520 - protein, 9 aa, Artificial Sequence
  Synthetic polypeptide
LPPPGCGSW

SEQ ID NO: 521 - protein, 10 aa, Artificial Sequence
  Synthetic polypeptide
HARGSSLITM

SEQ ID NO: 522 - protein, 9 aa, Artificial Sequence
  Synthetic polypeptide
MNKDLLRVL

SEQ ID NO: 523 - protein, 9 aa, Artificial Sequence
  Synthetic polypeptide
VAARWHGTL

SEQ ID NO: 524 - protein, 9 aa, Artificial Sequence
  Synthetic polypeptide
VAISFKTVF

SEQ ID NO: 525 - protein, 9 aa, Artificial Sequence
  Synthetic polypeptide
QTHWRLYPK

SEQ ID NO: 526 - protein, 9 aa, Artificial Sequence
  Synthetic polypeptide
LTMAPSCLR

SEQ ID NO: 527 - protein, 10 aa, Artificial Sequence
  Synthetic polypeptide
MPQACDGLTW

SEQ ID NO: 528 - protein, 9 aa, Artificial Sequence
  Synthetic polypeptide
SAWTGSVSV

SEQ ID NO: 529 - protein, 9 aa, Artificial Sequence
  Synthetic polypeptide
YPAASAPCW

SEQ ID NO: 530 - protein, 9 aa, Artificial Sequence
  Synthetic polypeptide
LPSSTLWTF

SEQ ID NO: 531 - protein, 9 aa, Artificial Sequence
  Synthetic polypeptide
LPCNVTFLM

SEQ ID NO: 532 - protein, 11 aa, Artificial Sequence
  Synthetic polypeptide
YPTSVHYQTPW

SEQ ID NO: 533 - protein, 9 aa, Artificial Sequence
  Synthetic polypeptide
NSLPPAALR

SEQ ID NO: 534 - protein, 9 aa, Artificial Sequence
  Synthetic polypeptide
VAHDPPQSL

SEQ ID NO: 535 - protein, 9 aa, Artificial Sequence
  Synthetic polypeptide
SASGSPWPM

SEQ ID NO: 536 - protein, 9 aa, Artificial Sequence
  Synthetic polypeptide
LLFPASGEM

SEQ ID NO: 537 - protein, 10 aa, Artificial Sequence
  Synthetic polypeptide
APAPPTRCVW

SEQ ID NO: 538 - protein, 9 aa, Artificial Sequence
  Synthetic polypeptide
SPQAPLRLW

SEQ ID NO: 539 - protein, 9 aa, Artificial Sequence
  Synthetic polypeptide
SSHPRPLPA

SEQ ID NO: 540 - protein, 10 aa, Artificial Sequence
  Synthetic polypeptide
SSARGWAPCK

SEQ ID NO: 541 - protein, 9 aa, Artificial Sequence
  Synthetic polypeptide
IAKKMTSTL

SEQ ID NO: 542 - protein, 9 aa, Artificial Sequence
  Synthetic polypeptide
RAMPSSGAA

SEQ ID NO: 543 - protein, 9 aa, Artificial Sequence
  Synthetic polypeptide
RTFCLTARR

SEQ ID NO: 544 - protein, 9 aa, Artificial Sequence
  Synthetic polypeptide
WACPHPRSL

SEQ ID NO: 545 - protein, 10 aa, Artificial Sequence
  Synthetic polypeptide
FGPSLVRGLW

SEQ ID NO: 546 - protein, 9 aa, Artificial Sequence
  Synthetic polypeptide
SASSLAAAL

SEQ ID NO: 547 - protein, 9 aa, Artificial Sequence
  Synthetic polypeptide
SMRSTRWAR

SEQ ID NO: 548 - protein, 10 aa, Artificial Sequence
  Synthetic polypeptide
ATLSAAFARR

SEQ ID NO: 549 - protein, 10 aa, Artificial Sequence
  Synthetic polypeptide
SPFPPSSLHW

SEQ ID NO: 550 - protein, 9 aa, Artificial Sequence
  Synthetic polypeptide
TAAACPTPV

SEQ ID NO: 551 - protein, 9 aa, Artificial Sequence
  Synthetic polypeptide
EAAPPSTSM

SEQ ID NO: 552 - protein, 10 aa, Artificial Sequence
  Synthetic polypeptide
TTQAPLRAAR

SEQ ID NO: 553 - protein, 9 aa, Artificial Sequence
  Synthetic polypeptide
RPFRTRMTW

SEQ ID NO: 554 - protein, 9 aa, Artificial Sequence
  Synthetic polypeptide
SPSMGAMRW

SEQ ID NO: 555 - protein, 10 aa, Artificial Sequence
  Synthetic polypeptide
CQPALLEQLF

SEQ ID NO: 556 - protein, 10 aa, Artificial Sequence
  Synthetic polypeptide
VSLPPPPLQR

SEQ ID NO: 557 - protein, 10 aa, Artificial Sequence
  Synthetic polypeptide
ATQEVQMRSR

SEQ ID NO: 558 - protein, 9 aa, Artificial Sequence
  Synthetic polypeptide
SIFPFQKTL

SEQ ID NO: 559 - protein, 9 aa, Artificial Sequence
  Synthetic polypeptide
SPHLPNPTW

SEQ ID NO: 560 - protein, 9 aa, Artificial Sequence
  Synthetic polypeptide
SVSWSIPRR

SEQ ID NO: 561 - protein, 10 aa, Artificial Sequence
  Synthetic polypeptide
LDTMSCPPWK

SEQ ID NO: 562 - protein, 9 aa, Artificial Sequence
  Synthetic polypeptide
RAGGMAPAK

SEQ ID NO: 563 - protein, 10 aa, Artificial Sequence
  Synthetic polypeptide
MASTVTEVLR

SEQ ID NO: 564 - protein, 9 aa, Artificial Sequence
  Synthetic polypeptide
TLQPASGSK

SEQ ID NO: 565 - protein, 10 aa, Artificial Sequence
  Synthetic polypeptide
RSRAPRTATR

SEQ ID NO: 566 - protein, 9 aa, Artificial Sequence
  Synthetic polypeptide
VAPTPQRPL

SEQ ID NO: 567 - protein, 9 aa, Artificial Sequence
  Synthetic polypeptide
HPLVATQAL

SEQ ID NO: 568 - protein, 9 aa, Artificial Sequence
  Synthetic polypeptide
QVWTAATLR

SEQ ID NO: 569 - protein, 9 aa, Artificial Sequence
  Synthetic polypeptide
LSASASTSL

SEQ ID NO: 570 - protein, 10 aa, Artificial Sequence
  Synthetic polypeptide
CAAWSSTRPW

SEQ ID NO: 571 - protein, 9 aa, Artificial Sequence
  Synthetic polypeptide
SAAAPRHSR

SEQ ID NO: 572 - protein, 9 aa, Artificial Sequence
  Synthetic polypeptide
FMQNIRIPI

SEQ ID NO: 573 - protein, 10 aa, Artificial Sequence
  Synthetic polypeptide
RKSHRFASGK

SEQ ID NO: 574 - protein, 9 aa, Artificial Sequence
  Synthetic polypeptide
STRPWTTSR

SEQ ID NO: 575 - protein, 9 aa, Artificial Sequence
  Synthetic polypeptide
TAAMGPHTY

SEQ ID NO: 576 - protein, 9 aa, Artificial Sequence
  Synthetic polypeptide
MTVKNLIQF

SEQ ID NO: 577 - protein, 11 aa, Artificial Sequence
  Synthetic polypeptide
LPQHAPHTLLY

SEQ ID NO: 578 - protein, 10 aa, Artificial Sequence
  Synthetic polypeptide
SMATVARAPR

SEQ ID NO: 579 - protein, 9 aa, Artificial Sequence
  Synthetic polypeptide
CLGLWCSPR

SEQ ID NO: 580 - protein, 8 aa, Artificial Sequence
  Synthetic polypeptide
QFFEIKSR

SEQ ID NO: 581 - protein, 10 aa, Artificial Sequence
  Synthetic polypeptide
WRPSVGLFSM

SEQ ID NO: 582 - protein, 9 aa, Artificial Sequence
  Synthetic polypeptide
HGQPVLSHR

SEQ ID NO: 583 - protein, 9 aa, Artificial Sequence
  Synthetic polypeptide
APSWSSRRW

SEQ ID NO: 584 - protein, 8 aa, Artificial Sequence
  Synthetic polypeptide
FSVMIPPM

SEQ ID NO: 585 - protein, 11 aa, Artificial Sequence
  Synthetic polypeptide
SPWTSRRGPPW

SEQ ID NO: 586 - protein, 10 aa, Artificial Sequence
  Synthetic polypeptide
ALRSPFLQGR

SEQ ID NO: 587 - protein, 10 aa, Artificial Sequence
  Synthetic polypeptide
KMERFWKLIR

SEQ ID NO: 588 - protein, 10 aa, Artificial Sequence
  Synthetic polypeptide
MNMKMKTFMK

SEQ ID NO: 589 - protein, 11 aa, Artificial Sequence
  Synthetic polypeptide
KGATYTPRHPR

SEQ ID NO: 590 - protein, 9 aa, Artificial Sequence
  Synthetic polypeptide
SSPTHVRAA

SEQ ID NO: 591 - protein, 9 aa, Artificial Sequence
  Synthetic polypeptide
KMWEPPMVL

SEQ ID NO: 592 - protein, 15 aa, Artificial Sequence
  Synthetic polypeptide
CQKKLMLLRLNLRKM

SEQ ID NO: 593 - protein, 15 aa, Artificial Sequence
  Synthetic polypeptide
DSQHVNLFLTKMMRM

SEQ ID NO: 594 - protein, 15 aa, Artificial Sequence
  Synthetic polypeptide
TYKVKAAASAHPLQM

SEQ ID NO: 595 - protein, 15 aa, Artificial Sequence
  Synthetic polypeptide
QLFVMSDTTYKIYWT

SEQ ID NO: 596 - protein, 15 aa, Artificial Sequence
  Synthetic polypeptide
EPVLSSLTSLRIELL

SEQ ID NO: 597 - protein, 15 aa, Artificial Sequence
  Synthetic polypeptide
RLIIQNLKSVRAYLQ

SEQ ID NO: 598 - protein, 15 aa, Artificial Sequence
  Synthetic polypeptide
LGGLIKLKLNRLNLI

SEQ ID NO: 599 - protein, 15 aa, Artificial Sequence
  Synthetic polypeptide
IISLFITKAYTLERK

SEQ ID NO: 600 - protein, 15 aa, Artificial Sequence
  Synthetic polypeptide
SILLHLIVLNSPESN

SEQ ID NO: 601 - protein, 15 aa, Artificial Sequence
  Synthetic polypeptide
SFLFLRQRATSTITI

SEQ ID NO: 602 - protein, 15 aa, Artificial Sequence
  Synthetic polypeptide
ELGILTSFGVQQKPR

SEQ ID NO: 603 - protein, 15 aa, Artificial Sequence
  Synthetic polypeptide
GNINLTFFTTKKSMI

SEQ ID NO: 604 - protein, 15 aa, Artificial Sequence
  Synthetic polypeptide
QARLCLIVSRTLLLV

SEQ ID NO: 605 - protein, 15 aa, Artificial Sequence
  Synthetic polypeptide
VSFWTLLPTTGVRTR

SEQ ID NO: 606 - protein, 15 aa, Artificial Sequence
  Synthetic polypeptide
RSLTCVLSVGRPLAT

SEQ ID NO: 607 - protein, 15 aa, Artificial Sequence
  Synthetic polypeptide
RPRLLLARTSQELAV

SEQ ID NO: 608 - protein, 15 aa, Artificial Sequence
  Synthetic polypeptide
GKLMHVLYFSSNEVT

SEQ ID NO: 609 - protein, 15 aa, Artificial Sequence
  Synthetic polypeptide
LQEWSRAISGGIAKR

SEQ ID NO: 610 - protein, 15 aa, Artificial Sequence
  Synthetic polypeptide
RGPLTSAPSAQQSLT

SEQ ID NO: 611 - protein, 15 aa, Artificial Sequence
  Synthetic polypeptide
KKNLCLLKSGQLWML

SEQ ID NO: 612 - protein, 15 aa, Artificial Sequence
  Synthetic polypeptide
KTEIQLTMNDSKHKL

SEQ ID NO: 613 - protein, 15 aa, Artificial Sequence
  Synthetic polypeptide
QKGILMLQNCHSYHL

SEQ ID NO: 614 - protein, 15 aa, Artificial Sequence
  Synthetic polypeptide
PGMLFFFATWSALVR

SEQ ID NO: 615 - protein, 15 aa, Artificial Sequence
  Synthetic polypeptide
KKKGLMTLSKMIKKK

SEQ ID NO: 616 - protein, 15 aa, Artificial Sequence
  Synthetic polypeptide
DQKILLLLEEKMIFR

SEQ ID NO: 617 - protein, 15 aa, Artificial Sequence
  Synthetic polypeptide
GTALIVHMTITKGKE

SEQ ID NO: 618 - protein, 15 aa, Artificial Sequence
  Synthetic polypeptide
VKPRKLTKVRFIKTL

SEQ ID NO: 619 - protein, 15 aa, Artificial Sequence
  Synthetic polypeptide
LIWKRVFILLLSDKK

SEQ ID NO: 620 - protein, 15 aa, Artificial Sequence
  Synthetic polypeptide
IVHFLLFKTSGRVQH

SEQ ID NO: 621 - protein, 15 aa, Artificial Sequence
  Synthetic polypeptide
PLRISLSLSNLSNSP

SEQ ID NO: 622 - protein, 15 aa, Artificial Sequence
  Synthetic polypeptide
VAWFYKSLWAPLLQR

SEQ ID NO: 623 - protein, 15 aa, Artificial Sequence
  Synthetic polypeptide
EKKLKKTPSLQTHQS

SEQ ID NO: 624 - protein, 15 aa, Artificial Sequence
  Synthetic polypeptide
MYSIRMENSTWMRPW

SEQ ID NO: 625 - protein, 15 aa, Artificial Sequence
  Synthetic polypeptide
GKNGILEDSQKKRRY

SEQ ID NO: 626 - protein, 15 aa, Artificial Sequence
  Synthetic polypeptide
WLKKKMLKNVRGRRK

SEQ ID NO: 627 - protein, 17 aa, Artificial Sequence
  Synthetic polypeptide
GSFRFFGSRMSVLSFSN

SEQ ID NO: 628 - protein, 15 aa, Artificial Sequence
  Synthetic polypeptide
VGKLVTLRNVSTKKY

SEQ ID NO: 629 - protein, 15 aa, Artificial Sequence
  Synthetic polypeptide
RMQLCTQLARFFPIT

SEQ ID NO: 630 - protein, 15 aa, Artificial Sequence
  Synthetic polypeptide
GTFMVIDCLSLRKKA

SEQ ID NO: 631 - protein, 15 aa, Artificial Sequence
  Synthetic polypeptide
LKTLFHLRGISGNLK

SEQ ID NO: 632 - protein, 15 aa, Artificial Sequence
  Synthetic polypeptide
IWNRIEPASTYRQYS

SEQ ID NO: 633 - protein, 15 aa, Artificial Sequence
  Synthetic polypeptide
FWQICHIKKHFQTHK

SEQ ID NO: 634 - protein, 15 aa, Artificial Sequence
  Synthetic polypeptide
NEWVKSDQVKKRKKR

SEQ ID NO: 635 - protein, 15 aa, Artificial Sequence
  Synthetic polypeptide
SIPWAPTSSRSVCPI

SEQ ID NO: 636 - protein, 15 aa, Artificial Sequence
  Synthetic polypeptide
ELRHVVPAPAHRGAL

SEQ ID NO: 637 - protein, 15 aa, Artificial Sequence
  Synthetic polypeptide
WGFLHLVSPSGTQYF

SEQ ID NO: 638 - protein, 15 aa, Artificial Sequence
  Synthetic polypeptide
LPTLRPTRPLQTVPL

SEQ ID NO: 639 - protein, 15 aa, Artificial Sequence
  Synthetic polypeptide
PAAYLLGLPGVQWLG

SEQ ID NO: 640 - protein, 15 aa, Artificial Sequence
  Synthetic polypeptide
LFFFVRQWGVQRVST

SEQ ID NO: 641 - protein, 15 aa, Artificial Sequence
  Synthetic polypeptide
SKMYTTSMAMPILPL

SEQ ID NO: 642 - protein, 15 aa, Artificial Sequence
  Synthetic polypeptide
DIGEVSQFLTEGIMK

SEQ ID NO: 643 - protein, 15 aa, Artificial Sequence
  Synthetic polypeptide
PLMIKNRISPLLTIL

SEQ ID NO: 644 - protein, 15 aa, Artificial Sequence
  Synthetic polypeptide
YFINFIYLAKSTKKP

SEQ ID NO: 645 - protein, 15 aa, Artificial Sequence
  Synthetic polypeptide
VLLQMFLRGLKRLLQ

SEQ ID NO: 646 - protein, 15 aa, Artificial Sequence
  Synthetic polypeptide
LWELRFHQDRGQRLP

SEQ ID NO: 647 - protein, 15 aa, Artificial Sequence
  Synthetic polypeptide
RPLKCMATLPNIRKF

SEQ ID NO: 648 - protein, 16 aa, Artificial Sequence
  Synthetic polypeptide
KEKKKNLAIVEEEMFL

SEQ ID NO: 649 - protein, 14 aa, Artificial Sequence
  Synthetic polypeptide
EPGVLAAAAETEHL

SEQ ID NO: 650 - protein, 15 aa, Artificial Sequence
  Synthetic polypeptide
YKKKAAICTTPALVK

SEQ ID NO: 651 - protein, 15 aa, Artificial Sequence
  Synthetic polypeptide
TLETLVKDLKKKSRV

SEQ ID NO: 652 - protein, 15 aa, Artificial Sequence
  Synthetic polypeptide
HVSVYPKRSFLCSSI

SEQ ID NO: 653 - protein, 15 aa, Artificial Sequence
  Synthetic polypeptide
RIRKKKVKSSVLQLK

SEQ ID NO: 654 - protein, 15 aa, Artificial Sequence
  Synthetic polypeptide
QPRLMILCSCLTMKE

SEQ ID NO: 655 - protein, 15 aa, Artificial Sequence
  Synthetic polypeptide
RIAFGMMSMASRCPA

SEQ ID NO: 656 - protein, 15 aa, Artificial Sequence
  Synthetic polypeptide
GLFYLLFCSKATECK

SEQ ID NO: 657 - protein, 15 aa, Artificial Sequence
  Synthetic polypeptide
LVQSVLSSRGVAQTR

SEQ ID NO: 658 - protein, 17 aa, Artificial Sequence
  Synthetic polypeptide
FPLAEKVKALADPSAFV

SEQ ID NO: 659 - protein, 15 aa, Artificial Sequence
  Synthetic polypeptide
AWHFSRAATEVTWWA

SEQ ID NO: 660 - protein, 15 aa, Artificial Sequence
  Synthetic polypeptide
RNSEITMQQIAVLLL

SEQ ID NO: 661 - protein, 15 aa, Artificial Sequence
  Synthetic polypeptide
LSLIMLAQAQEVFFF

SEQ ID NO: 662 - protein, 15 aa, Artificial Sequence
  Synthetic polypeptide
QEYLPLEFHKGYLLS

SEQ ID NO: 663 - protein, 15 aa, Artificial Sequence
  Synthetic polypeptide
VTNFLKATGLNIWKL

SEQ ID NO: 664 - protein, 15 aa, Artificial Sequence
  Synthetic polypeptide
GFELLRKNGLERRTR

SEQ ID NO: 665 - protein, 15 aa, Artificial Sequence
  Synthetic polypeptide
FSGICYLLSSVSFFL

SEQ ID NO: 666 - protein, 15 aa, Artificial Sequence
  Synthetic polypeptide
PGILQQKMQFRLLVL

SEQ ID NO: 667 - protein, 15 aa, Artificial Sequence
  Synthetic polypeptide
ATTIRLCWKASGRLA

SEQ ID NO: 668 - protein, 15 aa, Artificial Sequence
  Synthetic polypeptide
QRASALLASCSKKLH

SEQ ID NO: 669 - protein, 15 aa, Artificial Sequence
  Synthetic polypeptide
ESAVTALVPPPALSL

SEQ ID NO: 670 - protein, 15 aa, Artificial Sequence
  Synthetic polypeptide
ALPRIHNMSKAALRV

SEQ ID NO: 671 - protein, 17 aa, Artificial Sequence
  Synthetic polypeptide
ELQLSVLSAESLRENFS

SEQ ID NO: 672 - protein, 15 aa, Artificial Sequence
  Synthetic polypeptide
VSGWVVVKSEPIGPL

SEQ ID NO: 673 - protein, 15 aa, Artificial Sequence
  Synthetic polypeptide
RHRLNDIMTALLVQK

SEQ ID NO: 674 - protein, 15 aa, Artificial Sequence
  Synthetic polypeptide
PRRKTWKMRKKYAQK

SEQ ID NO: 675 - protein, 15 aa, Artificial Sequence
  Synthetic polypeptide
YLIKLLSRDLAKKKL

SEQ ID NO: 676 - protein, 15 aa, Artificial Sequence
  Synthetic polypeptide
PYLNTTGYPAPLHQE

SEQ ID NO: 677 - protein, 15 aa, Artificial Sequence
  Synthetic polypeptide
QFVISPPALRSRQKT

SEQ ID NO: 678 - protein, 15 aa, Artificial Sequence
  Synthetic polypeptide
QLGSGSSEASSVPHL

SEQ ID NO: 679 - protein, 15 aa, Artificial Sequence
  Synthetic polypeptide
PYFATYKAKMPLLRW

SEQ ID NO: 680 - protein, 15 aa, Artificial Sequence
  Synthetic polypeptide
FCKIKVSSAILSKKT

SEQ ID NO: 681 - protein, 15 aa, Artificial Sequence
  Synthetic polypeptide
AKVLLVRLKKNRSYL

SEQ ID NO: 682 - protein, 16 aa, Artificial Sequence
  Synthetic polypeptide
TAVTVMAGSVPSAQSV

SEQ ID NO: 683 - protein, 15 aa, Artificial Sequence
  Synthetic polypeptide
FYLGYNAMQDFFSYY

SEQ ID NO: 684 - protein, 15 aa, Artificial Sequence
  Synthetic polypeptide
NLLLKDQKPKKTRKI

SEQ ID NO: 685 - protein, 15 aa, Artificial Sequence
  Synthetic polypeptide
QDTVIIKKNPALPKH

SEQ ID NO: 686 - protein, 15 aa, Artificial Sequence
  Synthetic polypeptide
AGWVGKWAGLQLPFY

SEQ ID NO: 687 - protein, 15 aa, Artificial Sequence
  Synthetic polypeptide
LMLQHITLMCSAYRN

SEQ ID NO: 688 - protein, 15 aa, Artificial Sequence
  Synthetic polypeptide
KKKRKIIEFRIKFLF

SEQ ID NO: 689 - protein, 17 aa, Artificial Sequence
  Synthetic polypeptide
FLDIHNIHVMRESLKKS

SEQ ID NO: 690 - protein, 15 aa, Artificial Sequence
  Synthetic polypeptide
LRVLVLMNSKHTFLA

SEQ ID NO: 691 - protein, 15 aa, Artificial Sequence
  Synthetic polypeptide
ECIEVYGYHNIRVYK

SEQ ID NO: 692 - protein, 15 aa, Artificial Sequence
  Synthetic polypeptide
CFFFFCYILNTMFDR

SEQ ID NO: 693 - protein, 15 aa, Artificial Sequence
  Synthetic polypeptide
APLRLWSWCGTSQTY

SEQ ID NO: 694 - protein, 15 aa, Artificial Sequence
  Synthetic polypeptide
TRLMAPVGSVMSCSL

SEQ ID NO: 695 - protein, 15 aa, Artificial Sequence
  Synthetic polypeptide
FFFSVIFSTRCLTDS

SEQ ID NO: 696 - protein, 14 aa, Artificial Sequence
  Synthetic polypeptide
KKRKIIEFRIKFLF

SEQ ID NO: 697 - protein, 15 aa, Artificial Sequence
  Synthetic polypeptide
QVPALAPQAWWPARR

SEQ ID NO: 698 - protein, 15 aa, Artificial Sequence
  Synthetic polypeptide
EFLSHPFAVTLYGGG

SEQ ID NO: 699 - protein, 15 aa, Artificial Sequence
  Synthetic polypeptide
MSVCFFFLLYSQHDV

SEQ ID NO: 700 - protein, 15 aa, Artificial Sequence
  Synthetic polypeptide
PSQVWTAATLRCPAV

SEQ ID NO: 701 - protein, 15 aa, Artificial Sequence
  Synthetic polypeptide
RGRMLTWRSSLWLQG

SEQ ID NO: 702 - protein, 15 aa, Artificial Sequence
  Synthetic polypeptide
PQSLRPVRVRAHPGL

SEQ ID NO: 703 - protein, 15 aa, Artificial Sequence
  Synthetic polypeptide
RSKFTGLCRPLTSLA

SEQ ID NO: 704 - protein, 15 aa, Artificial Sequence
  Synthetic polypeptide
RSSTTAGLPARCAAW

SEQ ID NO: 705 - protein, 16 aa, Artificial Sequence
  Synthetic polypeptide
RGEAMGSGQATVTSMS

SEQ ID NO: 706 - protein, 15 aa, Artificial Sequence
  Synthetic polypeptide
NHIVVSAEGNISKKQ

SEQ ID NO: 707 - protein, 15 aa, Artificial Sequence
  Synthetic polypeptide
IPPLHLRTSARSKFT

SEQ ID NO: 708 - protein, 16 aa, Artificial Sequence
  Synthetic polypeptide
GEAMGSGQATVTSMSP

SEQ ID NO: 709 - protein, 15 aa, Artificial Sequence
  Synthetic polypeptide
TNMEIPHFFVILPKS

SEQ ID NO: 710 - protein, 15 aa, Artificial Sequence
  Synthetic polypeptide
KKKTYTCAITTVKAT

SEQ ID NO: 711 - protein, 15 aa, Artificial Sequence
  Synthetic polypeptide
NIDLCTALSALSGIP

SEQ ID NO: 712 - protein, 15 aa, Artificial Sequence
  Synthetic polypeptide
VPQLLHLPQFHSLRR

SEQ ID NO: 713 - protein, 15 aa, Artificial Sequence
  Synthetic polypeptide
SSLITMLTPRQKARK

SEQ ID NO: 714 - protein, 15 aa, Artificial Sequence
  Synthetic polypeptide
LQGFIQDRAGRMGGR

SEQ ID NO: 715 - protein, 15 aa, Artificial Sequence
  Synthetic polypeptide
REKIINPTISCPFQS

SEQ ID NO: 716 - protein, 15 aa, Artificial Sequence
  Synthetic polypeptide
VGTMSSSWRLWNKTR

SEQ ID NO: 717 - protein, 15 aa, Artificial Sequence
  Synthetic polypeptide
QTPASLMITRARKGR

SEQ ID NO: 718 - protein, 16 aa, Artificial Sequence
  Synthetic polypeptide
SPLPRGSGAAPLSWDS

SEQ ID NO: 719 - protein, 16 aa, Artificial Sequence
  Synthetic polypeptide
KTYTCAITTVKATETK

SEQ ID NO: 720 - protein, 15 aa, Artificial Sequence
  Synthetic polypeptide
QLLDLKSSLLKRPIH

SEQ ID NO: 721 - protein, 15 aa, Artificial Sequence
  Synthetic polypeptide
FRQDKLKVHMRKHTG

SEQ ID NO: 722 - protein, 18 aa, Artificial Sequence
  Synthetic polypeptide
VQSLLMYKDGDSVLQRGA

SEQ ID NO: 723 - protein, 16 aa, Artificial Sequence
  Synthetic polypeptide
IKGSESATYVPVAPPH

SEQ ID NO: 724 - protein, 15 aa, Artificial Sequence
  Synthetic polypeptide
EETQEKMTILQTYFR

SEQ ID NO: 725 - protein, 16 aa, Artificial Sequence
  Synthetic polypeptide
MLGNVESGGPHLLQPA

SEQ ID NO: 726 - protein, 16 aa, Artificial Sequence
  Synthetic polypeptide
ENALLNGSSFLVSSSI

SEQ ID NO: 727 - protein, 15 aa, Artificial Sequence
  Synthetic polypeptide
RPFFLPVYRQTHWRL

SEQ ID NO: 728 - protein, 15 aa, Artificial Sequence
  Synthetic polypeptide
MCTWLTMAPSCLRRS

SEQ ID NO: 729 - protein, 15 aa, Artificial Sequence
  Synthetic polypeptide
TLPVQRLRALSQNER

SEQ ID NO: 730 - protein, 15 aa, Artificial Sequence
  Synthetic polypeptide
PPAALRSGIPRLLHQ

SEQ ID NO: 731 - protein, 15 aa, Artificial Sequence
  Synthetic polypeptide
SGWRRLHRAMPSSGA

SEQ ID NO: 732 - protein, 15 aa, Artificial Sequence
  Synthetic polypeptide
PPPLQRLQGHLGRPY

SEQ ID NO: 733 - protein, 15 aa, Artificial Sequence
  Synthetic polypeptide
QAPFFCLRVRCGGWL

SEQ ID NO: 734 - protein, 16 aa, Artificial Sequence
  Synthetic polypeptide
VWMASASVTASTAGMT

SEQ ID NO: 735 - protein, 15 aa, Artificial Sequence
  Synthetic polypeptide
LVAISFKTVFKASHA

SEQ ID NO: 736 - protein, 15 aa, Artificial Sequence
  Synthetic polypeptide
NVTFLMQATLCARQE

SEQ ID NO: 737 - protein, 15 aa, Artificial Sequence
  Synthetic polypeptide
ASITSISRTTSSLSS

SEQ ID NO: 738 - protein, 16 aa, Artificial Sequence
  Synthetic polypeptide
QVIVSRGGALSGSPRL

SEQ ID NO: 739 - protein, 16 aa, Artificial Sequence
  Synthetic polypeptide
GVQGPSMATVARAPRA

SEQ ID NO: 740 - protein, 16 aa, Artificial Sequence
  Synthetic polypeptide
ESTPTASSAMAVTRSS

SEQ ID NO: 741 - protein, 16 aa, Artificial Sequence
  Synthetic polypeptide
TTLWLGPASVAPATLP

SEQ ID NO: 742 - protein, 16 aa, Artificial Sequence
  Synthetic polypeptide
SDGTPGGAPAQPACPR

SEQ ID NO: 743 - protein, 15 aa, Artificial Sequence
  Synthetic polypeptide
RLQGLAGAPRGRRSA

SEQ ID NO: 744 - protein, 15 aa, Artificial Sequence
  Synthetic polypeptide
GPELRRSRAPRTATR

SEQ ID NO: 745 - protein, 15 aa, Artificial Sequence
  Synthetic polypeptide
PSTAVATWRRCAGPA

SEQ ID NO: 746 - protein, 15 aa, Artificial Sequence
  Synthetic polypeptide
AMMNGKVPFFSALKV

SEQ ID NO: 747 - protein, 15 aa, Artificial Sequence
  Synthetic polypeptide
PLTSGGSAAATAKWG

SEQ ID NO: 748 - protein, 15 aa, Artificial Sequence
  Synthetic polypeptide
HTGTQFFEIKSRPLT

SEQ ID NO: 749 - protein, 15 aa, Artificial Sequence
  Synthetic polypeptide
LGQILILPPWAFLDP

SEQ ID NO: 750 - protein, 15 aa, Artificial Sequence
  Synthetic polypeptide
RSWRFLRAAPSSSAR

SEQ ID NO: 751 - protein, 15 aa, Artificial Sequence
  Synthetic polypeptide
GPQLLLSHRAVPHVH

SEQ ID NO: 752 - protein, 15 aa, Artificial Sequence
  Synthetic polypeptide
TNLRVQLLKRQLSLS

SEQ ID NO: 753 - protein, 15 aa, Artificial Sequence
  Synthetic polypeptide
QGEVDSQQGARAAAE

SEQ ID NO: 754 - protein, 15 aa, Artificial Sequence
  Synthetic polypeptide
AILLQVIAKKMTSTL

SEQ ID NO: 755 - protein, 15 aa, Artificial Sequence
  Synthetic polypeptide
LEQLFLCALKARAFR

SEQ ID NO: 756 - protein, 15 aa, Artificial Sequence
  Synthetic polypeptide
QERVRLIPRLRSLPR

SEQ ID NO: 757 - protein, 16 aa, Artificial Sequence
  Synthetic polypeptide
SPSTPGAATAAASSRP

SEQ ID NO: 758 - protein, 15 aa, Artificial Sequence
  Synthetic polypeptide
RTFCLTARRGALAIR

SEQ ID NO: 759 - protein, 15 aa, Artificial Sequence
  Synthetic polypeptide
EEVVCLLFPASGEMK

SEQ ID NO: 760 - protein, 15 aa, Artificial Sequence
  Synthetic polypeptide
RRRATLSAAFARRRF

SEQ ID NO: 761 - protein, 15 aa, Artificial Sequence
  Synthetic polypeptide
STLPRPFRTRMTWRS

SEQ ID NO: 762 - protein, 15 aa, Artificial Sequence
  Synthetic polypeptide
PMFLALDRRGGPGQA

SEQ ID NO: 763 - protein, 15 aa, Artificial Sequence
  Synthetic polypeptide
PTCLLLSRPLRSPHL

SEQ ID NO: 764 - protein, 15 aa, Artificial Sequence
  Synthetic polypeptide
LREWTTQAPLRAARW

SEQ ID NO: 765 - protein, 15 aa, Artificial Sequence
  Synthetic polypeptide
FLPWVPERGVASWTW

SEQ ID NO: 766 - protein, 15 aa, Artificial Sequence
  Synthetic polypeptide
VQMRSRMSSSARRMW

SEQ ID NO: 767 - protein, 16 aa, Artificial Sequence
  Synthetic polypeptide
GPLVPGLVLGGVREEE

SEQ ID NO: 768 - protein, 15 aa, Artificial Sequence
  Synthetic polypeptide
LPALSILQRSPRLPR

SEQ ID NO: 769 - protein, 15 aa, Artificial Sequence
  Synthetic polypeptide
AQSSWRSLEASALPV

SEQ ID NO: 770 - protein, 15 aa, Artificial Sequence
  Synthetic polypeptide
PESFKRQARARLERR

SEQ ID NO: 771 - protein, 15 aa, Artificial Sequence
  Synthetic polypeptide
SPVTQITGAAARQLL

SEQ ID NO: 772 - protein, 15 aa, Artificial Sequence
  Synthetic polypeptide
YAGGVGAQLMAPLSP

SEQ ID NO: 773 - protein, 15 aa, Artificial Sequence
  Synthetic polypeptide
VTELAQVIVSRGGGA

SEQ ID NO: 774 - protein, 15 aa, Artificial Sequence
  Synthetic polypeptide
PTHVRAAARVSSTAS

SEQ ID NO: 775 - protein, 15 aa, Artificial Sequence
  Synthetic polypeptide
RRPCCWMGAAAVWRG

SEQ ID NO: 776 - protein, 15 aa, Artificial Sequence
  Synthetic polypeptide
HDLGLHVLSCRIIPV

SEQ ID NO: 777 - protein, 40 aa, Artificial Sequence
  Synthetic polypeptide
WKAKREKMRAKQNPPGPAPPGGGSSDAAGKPPAGALGTPA

SEQ ID NO: 778 - protein, 40 aa, Artificial Sequence
  Synthetic polypeptide
QYQEEAEEEVQEDLTEEKKRELEHNAEETYGENDENTDDK

SEQ ID NO: 779 - protein, 40 aa, Artificial Sequence
  Synthetic polypeptide
GNKCTMCKEKLEREAAEKKKKEDEDRSNTGERSNTGERSN

SEQ ID NO: 780 - protein, 40 aa, Artificial Sequence
  Synthetic polypeptide
SKLRLAPDKLKSTESELKKKEKRRDEMLGLVPMRQSIIDL

SEQ ID NO: 781 - protein, 40 aa, Artificial Sequence
  Synthetic polypeptide
RGEGRFGVSRRRHNSSDGFFNNGPLRTAGDSWHQPSLFRH

SEQ ID NO: 782 - protein, 40 aa, Artificial Sequence
  Synthetic polypeptide
DLINKFGTLNGFQILHDRFFNGSALNIQIIAALIKPFGQC

SEQ ID NO: 783 - protein, 40 aa, Artificial Sequence
  Synthetic polypeptide
PVKVLVGKNFEDVAFDEKKNVFVEFYAPWCGHCKQLAPIW

SEQ ID NO: 784 - protein, 40 aa, Artificial Sequence
  Synthetic polypeptide
FNLQKSSLSARHPQRKRRGGPSEPTPGSRPQDATVHPACQ

SEQ ID NO: 785 - protein, 40 aa, Artificial Sequence
  Synthetic polypeptide
CAGPQTYKEHLEGQKHKKKEAALKASQNTSSSNSSTRGTQ

SEQ ID NO: 786 - protein, 40 aa, Artificial Sequence
  Synthetic polypeptide
DLPIDDKLDNQCVSVEPKKKEQENKTLVLSDKHSPQKKST

SEQ ID NO: 787 - protein, 37 aa, Artificial Sequence
  Synthetic polypeptide
NFSVRCPKHKPPLPCPLPPLQNKTAKGSLSTEQSERG

SEQ ID NO: 788 - protein, 40 aa, Artificial Sequence
  Synthetic polypeptide
ALFGLDRQTLWCKPCRRKKKCVRYIQGEGSCLSPPSSDGS

SEQ ID NO: 789 - protein, 40 aa, Artificial Sequence
  Synthetic polypeptide
DQLQQAVQSQGFINYCQKKIDASQTEFEKNVWSFLKVNFE

SEQ ID NO: 790 - protein, 40 aa, Artificial Sequence
  Synthetic polypeptide
LAEPPHFVEHIRSTLMFLKKHPSPAHTLFSGNKALLYKKN

SEQ ID NO: 791 - protein, 40 aa, Artificial Sequence
  Synthetic polypeptide
LSSSGFLDASDPALQPPGGVPSSLAESHLCLPSAFALSIP

SEQ ID NO: 792 - protein, 40 aa, Artificial Sequence
  Synthetic polypeptide
PTVALSAVAGASQVKWNKKNANCLATSHDGDVRIWDKRKP

SEQ ID NO: 793 - protein, 40 aa, Artificial Sequence
  Synthetic polypeptide
GFGLGKVSYIGVCQSKFHFFEDQLRGAGFGPQHNRHCLLT

SEQ ID NO: 794 - protein, 40 aa, Artificial Sequence
  Synthetic polypeptide
SKVRGISEVLARRHMKVAFFGRTSNGKSTVINAMLWDKVL

SEQ ID NO: 795 - protein, 40 aa, Artificial Sequence
  Synthetic polypeptide
EPGPLGGGGSGGPQMGLPPPPPALRPRLVFHTQLAHGSPT

SEQ ID NO: 796 - protein, 40 aa, Artificial Sequence
  Synthetic polypeptide
EEAYRCNFLGLSPHVQIPPHVLSSEFAVIVEVHAAARSTL

SEQ ID NO: 797 - protein, 40 aa, Artificial Sequence
  Synthetic polypeptide
MSYAANLKNVMNMQNRQKKEGEEQPVLPEETESSKPGPSA

SEQ ID NO: 798 - protein, 40 aa, Artificial Sequence
  Synthetic polypeptide
HKDAWRQPEDTWAALEGLSFSPFREQMLDTSSLLQFMREK

SEQ ID NO: 799 - protein, 40 aa, Artificial Sequence
  Synthetic polypeptide
IIYNQGFEIVLNDYKWFAFFKYKEEGSKVTTYCNETMTGW

SEQ ID NO: 800 - protein, 40 aa, Artificial Sequence
  Synthetic polypeptide
LKSQKQVKVEMSGPVTVLTRQTTAAELDSHTPALEQQTTS

SEQ ID NO: 801 - protein, 40 aa, Artificial Sequence
  Synthetic polypeptide
DEAFDTANSSIVSGESIRFFVNVNLEMQATNTENEATSGG

SEQ ID NO: 802 - protein, 40 aa, Artificial Sequence
  Synthetic polypeptide
KIREVRQKIMQAATPTEQPPGAEAPLPVPPPTGTAAAPAP

SEQ ID NO: 803 - protein, 40 aa, Artificial Sequence
  Synthetic polypeptide
EINVIIKNPEIVFVADMTKNDAPALVITTQCEICYKGNLE

SEQ ID NO: 804 - protein, 40 aa, Artificial Sequence
  Synthetic polypeptide
ATYVTFSPNGTELLVNMGGEQVYLFDLTYKQRPYTFLLPR

SEQ ID NO: 805 - protein, 40 aa, Artificial Sequence
  Synthetic polypeptide
PLMILDEERELEKLFQLGPPSPVKMPSPPWESNLLQSPSS

SEQ ID NO: 806 - protein, 40 aa, Artificial Sequence
  Synthetic polypeptide
KSKGPKKTAKSKKKKPLKKKPTPVLLPQSKQQKQKQANGV

SEQ ID NO: 807 - protein, 40 aa, Artificial Sequence
  Synthetic polypeptide
RNLAFFQLRKVWGQVWHSIQTLKEDCNRLQQGQRAAMMNL

SEQ ID NO: 808 - protein, 40 aa, Artificial Sequence
  Synthetic polypeptide
PKAARIKEVLKERKVLEKKVALSKKRKKDSRNVEENSKKK

SEQ ID NO: 809 - protein, 40 aa, Artificial Sequence
  Synthetic polypeptide
LKQKFSMKAQNGFNKKRKKNVFNPKRVVEDSEYDSGSDVG

SEQ ID NO: 810 - protein, 40 aa, Artificial Sequence
  Synthetic polypeptide
IIKCIEDIKRPGEWSGLEKNKKDGFKSSQLNNPQFVWVVP

SEQ ID NO: 811 - protein, 40 aa, Artificial Sequence
  Synthetic polypeptide
EKVASDTEEADRTSSKKTKTQEISRPNSPSEGEGESSDSR

SEQ ID NO: 812 - protein, 40 aa, Artificial Sequence
  Synthetic polypeptide
TANMKASENLKHIVNHDDVFEESEELSSDEEMKMAEMRPP

SEQ ID NO: 813 - protein, 40 aa, Artificial Sequence
  Synthetic polypeptide
SCLIKYNVSTTPYLQSVKKKVQFDGTNSAFKELKFLTPVR

SEQ ID NO: 814 - protein, 40 aa, Artificial Sequence
  Synthetic polypeptide
LFKPADVILDPDTANAILLVSEDQRSVQRAEEPRDLPDNP

SEQ ID NO: 815 - protein, 40 aa, Artificial Sequence
  Synthetic polypeptide
SMVDEVSGKVLEMDISKKKALQQKDIHKKIKQNESATDEI

SEQ ID NO: 816 - protein, 40 aa, Artificial Sequence
  Synthetic polypeptide
VQERKIPAHRVVLAAASHFFNLMFTTNMLESKSFEVELKD

SEQ ID NO: 817 - protein, 40 aa, Artificial Sequence
  Synthetic polypeptide
SFGSPTGNQMSSDIDEYKKNIHGNALRTSGSSSSDVTKAS

SEQ ID NO: 818 - protein, 40 aa, Artificial Sequence
  Synthetic polypeptide
IIFNFEKAYFILDEFLMGGDVQDTSKKSVLKAIEQADLLQ

SEQ ID NO: 819 - protein, 40 aa, Artificial Sequence
  Synthetic polypeptide
EDSPYETLHSFISNAVAPFFKSYIRESGKADRDGDKMAPS

SEQ ID NO: 820 - protein, 40 aa, Artificial Sequence
  Synthetic polypeptide
GWGPPPPPPPLLPCTCSPPVAGGMEEVIVAQVDHGLGSAW

SEQ ID NO: 821 - protein, 40 aa, Artificial Sequence
  Synthetic polypeptide
KKFKEEKKLKAKLKKVKKKRRRDEELSSEESPRRHHHQTK

SEQ ID NO: 822 - protein, 40 aa, Artificial Sequence
  Synthetic polypeptide
CSIERADNDKEYLVLTLTKNDLDKANKDKANRYFSPNFKV

SEQ ID NO: 823 - protein, 40 aa, Artificial Sequence
  Synthetic polypeptide
YATNPPWIFTQEAPEEGTGGFDGIYYGDNRFNTVSESGTA

SEQ ID NO: 824 - protein, 40 aa, Artificial Sequence
  Synthetic polypeptide
FTKTPKSSSPALKPKPNPPSPENTASSAPVDWRDPSQMEK

SEQ ID NO: 825 - protein, 37 aa, Artificial Sequence
  Synthetic polypeptide
MPSVCLLLLLFLAVGGALGNRPFRAFVVTDTTLTHLA

SEQ ID NO: 826 - protein, 40 aa, Artificial Sequence
  Synthetic polypeptide
GVYSRYFTTYDTNGRYSVKVRALGGVNAARRRVIPQQSGA

SEQ ID NO: 827 - protein, 40 aa, Artificial Sequence
  Synthetic polypeptide
DVKEETKEWLKNRIIAKKKDGGAQLLFRPLLNKYEQETLE

SEQ ID NO: 828 - protein, 40 aa, Artificial Sequence
  Synthetic polypeptide
LLDETSAITLTSTWKEVKKIIKEDPRCIKFSSSDRKKQRE

SEQ ID NO: 829 - protein, 40 aa, Artificial Sequence
  Synthetic polypeptide
VHSSSEPLRNLHLDIGALGGDFEYEESLRTSQPEEKKDVS

SEQ ID NO: 830 - protein, 40 aa, Artificial Sequence
  Synthetic polypeptide
STSYLLCISENKENVRDKKKGNIFIGIVGVQPATGEVVFD

SEQ ID NO: 831 - protein, 40 aa, Artificial Sequence
  Synthetic polypeptide
AQRKSSMNQLQQWVNLRRGVPPPEDLRSPSRFYPVSRRVP

SEQ ID NO: 832 - protein, 40 aa, Artificial Sequence
  Synthetic polypeptide
KLDQGEYERAAIDAVDNKKNTPLHYAAASGMKACVELLVK

SEQ ID NO: 833 - protein, 40 aa, Artificial Sequence
  Synthetic polypeptide
EKEERRVWTMPPMAVALKPVLQQSREARDELPGAPPVLCS

SEQ ID NO: 834 - protein, 40 aa, Artificial Sequence
  Synthetic polypeptide
ACGETLSVTSEENSLVKKKERSLSSGSNFCSEQKTSGIIN

SEQ ID NO: 835 - protein, 40 aa, Artificial Sequence
  Synthetic polypeptide
NPQRAQLCEDHCVDGCFCPPGTVLDDITHSGCLPLGQCPC

SEQ ID NO: 836 - protein, 40 aa, Artificial Sequence
  Synthetic polypeptide
SLRQQEERKRLYQRQQERGGIIDLEAERNRYFISLQQPPA

SEQ ID NO: 837 - protein, 40 aa, Artificial Sequence
  Synthetic polypeptide
SSKHNVIVGRNGSGKSNFFYAIQFVLSDEFSHLRPEQRLA

SEQ ID NO: 838 - protein, 28 aa, Artificial Sequence
  Synthetic polypeptide
MHGGGPPSGDSACPLRTIKRVQFGVLSP

SEQ ID NO: 839 - protein, 40 aa, Artificial Sequence
  Synthetic polypeptide
NQTTQEADSANTLQIAEIKEKIGTRSAEDPVSEVPAVSQH

SEQ ID NO: 840 - protein, 40 aa, Artificial Sequence
  Synthetic polypeptide
EREAGGPLPPSPLPHSSPPTAAVATTSITTATPGVPGLPS

SEQ ID NO: 841 - protein, 40 aa, Artificial Sequence
  Synthetic polypeptide
KAYLKQAPPSKGPTVRTKKVGKNEAVLEWDQLPVDVQNGF

SEQ ID NO: 842 - protein, 40 aa, Artificial Sequence
  Synthetic polypeptide
PISIIDQGEPKGTGATCGKKGSQAGAEGQPSTVKRYTPAR

SEQ ID NO: 843 - protein, 40 aa, Artificial Sequence
  Synthetic polypeptide
GSRLGNSLLLKYTEKLQEPPASAVREAADKEEPPSKKKRV

SEQ ID NO: 844 - protein, 40 aa, Artificial Sequence
  Synthetic polypeptide
EGGNLPDAAEPDIISTIKKTFNFGQNEALHLFQTLMECMK

SEQ ID NO: 845 - protein, 40 aa, Artificial Sequence
  Synthetic polypeptide
PPPEDSPMSPPPEESPMSPPPEVSRLSPLPVVSRLSPPPE

SEQ ID NO: 846 - protein, 40 aa, Artificial Sequence
  Synthetic polypeptide
FNYNRAFQVWAVPLLLVAFFAYLVAHSFLSVFETVLDALF

SEQ ID NO: 847 - protein, 40 aa, Artificial Sequence
  Synthetic polypeptide
SDEKRLCLQLLSDVLRGQGEAGQLEEAFSLALLPQLVVSL

SEQ ID NO: 848 - protein, 40 aa, Artificial Sequence
  Synthetic polypeptide
RKEEAFRHKLAMDTYSGPPPGPGPGPALPAHSSPGLPPPA

SEQ ID NO: 849 - protein, 40 aa, Artificial Sequence
  Synthetic polypeptide
REEKEKLFNEHIEALTKKKREHFRQLLDETSAITLTSTWK

SEQ ID NO: 850 - protein, 40 aa, Artificial Sequence
  Synthetic polypeptide
PYPQGGYPQGPYPQSPFPPNPYGQPQVFPGQDPDSPQHGN

SEQ ID NO: 851 - protein, 40 aa, Artificial Sequence
  Synthetic polypeptide
RGLAQADGTLITCVDSGILRVWHDKDKDTSSDPLLELRVG

SEQ ID NO: 852 - protein, 40 aa, Artificial Sequence
  Synthetic polypeptide
SIKYNEEKRHVDQPIDYSLKYATDIPSSQKQSFSFSKSSS

SEQ ID NO: 853 - protein, 40 aa, Artificial Sequence
  Synthetic polypeptide
QVMKYILDKIDKEEKQAAKKRKREESVEQKRSKQNATKLS

SEQ ID NO: 854 - protein, 40 aa, Artificial Sequence
  Synthetic polypeptide
RLESFLLQTGYAAGKGVGGGSADLIRNLRSRVDPQAPDLP

SEQ ID NO: 855 - protein, 40 aa, Artificial Sequence
  Synthetic polypeptide
DGKFANLTPSRTVPDSEAPPGWDRADSGPTQPPLSLSPAP

SEQ ID NO: 856 - protein, 40 aa, Artificial Sequence
  Synthetic polypeptide
DGTFSVTSAYSSAPDGSPPPAPLPASEMTMEDMAPGQLSS

SEQ ID NO: 857 - protein, 40 aa, Artificial Sequence
  Synthetic polypeptide
GGPQDPQPGLTAHVVSAGGRAEMHCFSIMVTPDPSTPSRL

SEQ ID NO: 858 - protein, 40 aa, Artificial Sequence
  Synthetic polypeptide
KLDYAVAWFIRESMTIYIFLSALWDPTISWRTGRYRLRCG

SEQ ID NO: 859 - protein, 40 aa, Artificial Sequence
  Synthetic polypeptide
VNQEVLEILDFHLYGSYPPGTPALKAYWENTYDAADGPSG

SEQ ID NO: 860 - protein, 40 aa, Artificial Sequence
  Synthetic polypeptide
MAVGASIAARLGTYPDWFFFCSFIGMFVFYCAHWQTYVSG

SEQ ID NO: 861 - protein, 40 aa, Artificial Sequence
  Synthetic polypeptide
RYPRKKFWVGKPIARVVKKKTGEFSDKLLSLQRGLREFQG

SEQ ID NO: 862 - protein, 40 aa, Artificial Sequence
  Synthetic polypeptide
LPPVFGEEYEEQPRPRSKKKGAKRKAVSGYQSHDDSSDNS

SEQ ID NO: 863 - protein, 40 aa, Artificial Sequence
  Synthetic polypeptide
FRQRKAQSDGQSPSKKQKKKRKTSSSKHDVSAHHDLNIDQ

SEQ ID NO: 864 - protein, 40 aa, Artificial Sequence
  Synthetic polypeptide
QVFALLFVTEYLTKWPKFFFDILSVVDLNPRGVDLYLRIL

SEQ ID NO: 865 - protein, 40 aa, Artificial Sequence
  Synthetic polypeptide
TILYFPFSSHSSYTVRSKKIFLSKLIVCFLSTWLPFVLLQ

SEQ ID NO: 866 - protein, 40 aa, Artificial Sequence
  Synthetic polypeptide
LNIKKISEEEYVALGSFFFWKCLHGESSTEDMCHTLESAG

SEQ ID NO: 867 - protein, 40 aa, Artificial Sequence
  Synthetic polypeptide
PYHWSPSRKAGRSDSSSSGGGGSPSEASGLGLDFEDSVWK

SEQ ID NO: 868 - protein, 40 aa, Artificial Sequence
  Synthetic polypeptide
GASLYVGWAASGLLLLGGGLLCCNCPPRTDKPYSAKYSAA

SEQ ID NO: 869 - protein, 40 aa, Artificial Sequence
  Synthetic polypeptide
PGPCGPPPGHGPGPCGPPPHHGPGPCGPPPGHGPGHPPPG

SEQ ID NO: 870 - protein, 40 aa, Artificial Sequence
  Synthetic polypeptide
ESHSLSAHLQLTFTGFFHKNDKPSPNSENEQNSVTLEVLL

SEQ ID NO: 871 - protein, 40 aa, Artificial Sequence
  Synthetic polypeptide
VPNTDQKSTSVKKDNHKKKTVKMLEYLGKDVLHGVFNYLA

SEQ ID NO: 872 - protein, 40 aa, Artificial Sequence
  Synthetic polypeptide
QLKEMCRRELDKAESEIKKNSSIIGDYKQICSQLSERLEK

SEQ ID NO: 873 - protein, 40 aa, Artificial Sequence
  Synthetic polypeptide
QTEMRVQLLQDLQDFFRKKAEIETEYSRNLEKLAERFMAK

SEQ ID NO: 874 - protein, 40 aa, Artificial Sequence
  Synthetic polypeptide
DVLFVYAVRECCKCIDGKKVGKELTEKPKFILSVLLLWNF

SEQ ID NO: 875 - protein, 40 aa, Artificial Sequence
  Synthetic polypeptide
TVYEFLLMKVEKDHLAKPFFPAIYKEFEELHKMVKKMCQD

SEQ ID NO: 876 - protein, 40 aa, Artificial Sequence
  Synthetic polypeptide
CESKLYSLDHGHEKPQDKKKRTSGLATLKKKFIKRRKSNR

SEQ ID NO: 877 - protein, 40 aa, Artificial Sequence
  Synthetic polypeptide
KGHYSLHFDAFHHPLGDTPPALPARTLRKSPLHPIPASPT

SEQ ID NO: 878 - protein, 40 aa, Artificial Sequence
  Synthetic polypeptide
PLGQSHLAHHSMAPYPFPPNPDMNPELRKALLQDSAPQPA

SEQ ID NO: 879 - protein, 40 aa, Artificial Sequence
  Synthetic polypeptide
KNISSEHSMSSTPLTIGEKNRNSINYERQQAQARIPSPET

SEQ ID NO: 880 - protein, 40 aa, Artificial Sequence
  Synthetic polypeptide
EPNASVVPPPLPATWMRPPREPAQPPREEVRKSFVESVEE

SEQ ID NO: 881 - protein, 40 aa, Artificial Sequence
  Synthetic polypeptide
SYAPAEIFLPKGRSNSKKKRQKKQNTSCSKNRGRTTAHTK

SEQ ID NO: 882 - protein, 40 aa, Artificial Sequence
  Synthetic polypeptide
SSSGAFSLLGRFCGAEPPPHLVSSHHELAVLFRTDHGISS

SEQ ID NO: 883 - protein, 40 aa, Artificial Sequence
  Synthetic polypeptide
TIMNRRLCCILVALSWMGGFIHSIIQVALIVRLPFCGPNE

SEQ ID NO: 884 - protein, 40 aa, Artificial Sequence
  Synthetic polypeptide
HDFILEDAASPKCIMKEKKKPGETFFMCSCSSDECNDNII

SEQ ID NO: 885 - protein, 40 aa, Artificial Sequence
  Synthetic polypeptide
PVAVPAPQQADGNPDVPPPRPLQGRSEREFFVKWVGLSYW

SEQ ID NO: 886 - protein, 40 aa, Artificial Sequence
  Synthetic polypeptide
LGRVQEFDSGLLHWRIGGGDTTEHIQTHFESKTELLPSRP

SEQ ID NO: 887 - protein, 40 aa, Artificial Sequence
  Synthetic polypeptide
GGNESQPDSQEDPREVLKKTLEFCLSRENLASDMYLISQM

SEQ ID NO: 888 - protein, 40 aa, Artificial Sequence
  Synthetic polypeptide
PKAEDGATPSPSNETPKKKKKRFSFKKSFKLSGFSFKKNK

SEQ ID NO: 889 - protein, 40 aa, Artificial Sequence
  Synthetic polypeptide
EIGQHPSLEDMQEVVVHKKKRPVLRDYWQKHAGMAMLCET

SEQ ID NO: 890 - protein, 40 aa, Artificial Sequence
  Synthetic polypeptide
ATIMNQRLCCILVALSWRGGFIHSIIQVALIVRLPFCGPN

SEQ ID NO: 891 - protein, 40 aa, Artificial Sequence
  Synthetic polypeptide
HIRKQQMVSVEETIYIVGGCLHELGPNRRSSQSEDMLTVQ

SEQ ID NO: 892 - protein, 40 aa, Artificial Sequence
  Synthetic polypeptide
SDFDRVGGMNTEEFRDQWGGEDWELLDRVLQAGLEVERLR

SEQ ID NO: 893 - protein, 40 aa, Artificial Sequence
  Synthetic polypeptide
TGIFRLTNAGMLEVSACKKKGFHPHTKEPRLFSICKHVLV

SEQ ID NO: 894 - protein, 40 aa, Artificial Sequence
  Synthetic polypeptide
VPRVEGVFIFLIEDSGKKKRRKNFEAMFKGILQSGLDNFV

SEQ ID NO: 895 - protein, 40 aa, Artificial Sequence
  Synthetic polypeptide
NKHAAFSCPKKPLSPPKKKVSHSSKKGGHSSPASSDKNSN

SEQ ID NO: 896 - protein, 40 aa, Artificial Sequence
  Synthetic polypeptide
GENARLHYRLVDTASTFLGGGSAGPKNPAPTPDFPFQIHN

SEQ ID NO: 897 - protein, 40 aa, Artificial Sequence
  Synthetic polypeptide
ESKFKSRASNAQAKPSSFFLQMQKRVSGHYVTSAAAKSVH

SEQ ID NO: 898 - protein, 40 aa, Artificial Sequence
  Synthetic polypeptide
SSQLLTPAERPGGLDDRSPPGSSETVELVRYEPDLLRLLG

SEQ ID NO: 899 - protein, 40 aa, Artificial Sequence
  Synthetic polypeptide
NCHISLTPNGDMPGSEIPPSSPSHAGSLHDDLNQVSRDDA

SEQ ID NO: 900 - protein, 40 aa, Artificial Sequence
  Synthetic polypeptide
ESIGDYYACARLSCAPPPPIHAINRGIFVEGLSCVLDGIF

SEQ ID NO: 901 - protein, 40 aa, Artificial Sequence
  Synthetic polypeptide
DLGLCVAELELLSSWFSPPTVVAGRRKSVDQPEGTPVELY

SEQ ID NO: 902 - protein, 40 aa, Artificial Sequence
  Synthetic polypeptide
TLEILKSTMKKELEAAQKKKPSLCEMLHMPNICKRISLLS

SEQ ID NO: 903 - protein, 40 aa, Artificial Sequence
  Synthetic polypeptide
PCFYPDEDDFYFGGPDSTPPGEDIWKKFELLPTPPLSPSR

SEQ ID NO: 904 - protein, 40 aa, Artificial Sequence
  Synthetic polypeptide
TGALLLQGFIQDRAGRMGGEAPELALDPVPQDASTKKLSE

SEQ ID NO: 905 - protein, 40 aa, Artificial Sequence
  Synthetic polypeptide
STSRSRPSRIPQPVRHHPPVLVSSAASSQAEADKMSGTST

SEQ ID NO: 906 - protein, 40 aa, Artificial Sequence
  Synthetic polypeptide
NQEFLQARTPTLASTPIPPTPQAPSPAVDAEIRAQDAPLS

SEQ ID NO: 907 - protein, 21 aa, Artificial Sequence
  Synthetic polypeptide
GAGPSGETGAAGVDGGCGGRH

SEQ ID NO: 908 - protein, 40 aa, Artificial Sequence
  Synthetic polypeptide
GQEQTFGILNVLEFSSDRKRMSVIVRTPSGRLRLYCKGAD

SEQ ID NO: 909 - protein, 40 aa, Artificial Sequence
  Synthetic polypeptide
ETMKQARHRLASFKTVIKKKGSVFPDDGRKSFLTREEVLS

SEQ ID NO: 910 - protein, 40 aa, Artificial Sequence
  Synthetic polypeptide
DFFRAQTLKETSLTNTMGGYKESFSSIMCFGCHDVYSQAS

SEQ ID NO: 911 - protein, 40 aa, Artificial Sequence
  Synthetic polypeptide
GRCKSGFDPRHGSHNIKKKAWYLIAMLLKLAFCLALCAKL

SEQ ID NO: 912 - protein, 40 aa, Artificial Sequence
  Synthetic polypeptide
DGLRSRVKYGVKTTPESPPYSSGSYDSIKTEVSGCPEDLT

SEQ ID NO: 913 - protein, 40 aa, Artificial Sequence
  Synthetic polypeptide
SIRTTDFHNPGYPKYLGTPHLELYLSDSLRNLNKERQFHF

SEQ ID NO: 914 - protein, 40 aa, Artificial Sequence
  Synthetic polypeptide
REPAGLSLVLKKIPIPETPPQTPPQVLDSPHQRSPSLSLA

SEQ ID NO: 915 - protein, 40 aa, Artificial Sequence
  Synthetic polypeptide
PQGLPGVKGDKGSPGKTGPRGKVGDPGVAGLPGEKGEKGE

SEQ ID NO: 916 - protein, 40 aa, Artificial Sequence
  Synthetic polypeptide
TAPSLQNNQPVEFNHAINYVNKIKNRFQGQPDIYKAFLEI

SEQ ID NO: 917 - protein, 40 aa, Artificial Sequence
  Synthetic polypeptide
AVSTGVQAGIPMPCFTTALSFYDGYRHEMLPASLIQAQRD

SEQ ID NO: 918 - protein, 40 aa, Artificial Sequence
  Synthetic polypeptide
CTHCYCLQGQTLCSTVSCPPLPCVEPINVEGSCCPMCPEM

SEQ ID NO: 919 - protein, 40 aa, Artificial Sequence
  Synthetic polypeptide
GTMRATGDFVTVKDGEIFFLGRKDSQIKRHGKRLNIELVQ

SEQ ID NO: 920 - protein, 40 aa, Artificial Sequence
  Synthetic polypeptide
LSLEINRKLQAVLEDTLLKNITLKENLQTLGTEIERLIKH

SEQ ID NO: 921 - protein, 40 aa, Artificial Sequence
  Synthetic polypeptide
PMAFSPQRDRFQAEGSLKKNEQNFKLAGVKKDIEKLYEAV

SEQ ID NO: 922 - protein, 34 aa, Artificial Sequence
  Synthetic polypeptide
LTDRQVKIWFQNRRMKEKKINRDRLQYYSANPLL

SEQ ID NO: 923 - protein, 40 aa, Artificial Sequence
  Synthetic polypeptide
GSAAAGGPTSYGTLKEPSGGGGTALLNKENKFRDRSFSEN

SEQ ID NO: 924 - protein, 40 aa, Artificial Sequence
  Synthetic polypeptide
GRVGPRRQRKHCITEDTPPTSLYIEGLDSKEAGGQSSQEE

SEQ ID NO: 925 - protein, 40 aa, Artificial Sequence
  Synthetic polypeptide
GAKIQWLKDAQGLPGGGGGDNSGTAENGRHSDLAALYTIV

SEQ ID NO: 926 - protein, 40 aa, Artificial Sequence
  Synthetic polypeptide
VRIAAPGIGVWNPAFDVTPHDLITGGIITELGVFAPEELR

SEQ ID NO: 927 - protein, 40 aa, Artificial Sequence
  Synthetic polypeptide
FRSKQEALKQGWLHKKGGGSSTLSRRNWKKRWFVLRQSKL

SEQ ID NO: 928 - protein, 40 aa, Artificial Sequence
  Synthetic polypeptide
EHVENAGVHSGDATLVTPPQDITAKTLERIKAIVHAVGQE

SEQ ID NO: 929 - protein, 40 aa, Artificial Sequence
  Synthetic polypeptide
QNYTNWSTSPYFLEHGIPPSCCMNETDCNPQDLHNLTVAA

SEQ ID NO: 930 - protein, 40 aa, Artificial Sequence
  Synthetic polypeptide
PTGPKNMQTSGRLSNVAPPCILRKNPPSARNGGHETDAQI

SEQ ID NO: 931 - protein, 40 aa, Artificial Sequence
  Synthetic polypeptide
DFLSVKWEAAMMNGKVPFFFSSESLGYFATGRPADNVMTT

SEQ ID NO: 932 - protein, 40 aa, Artificial Sequence
  Synthetic polypeptide
GCGFCNQDRRTLPGGQPPPRVFLAVFVEQPTPFLPRFLQR

SEQ ID NO: 933 - protein, 40 aa, Artificial Sequence
  Synthetic polypeptide
VLQTGTQRTIAPRTHPYSPKIDGTRTPRDERRRAQHNEVE

SEQ ID NO: 934 - protein, 40 aa, Artificial Sequence
  Synthetic polypeptide
AMDRNSLQSAVSNIAQAPLFIPPNSDPVPARDYLILKGVL

SEQ ID NO: 935 - protein, 40 aa, Artificial Sequence
  Synthetic polypeptide
DSCLLAAMAYDCYVAIRHPLPYATRMSRAMCAALVGMAWL

SEQ ID NO: 936 - protein, 40 aa, Artificial Sequence
  Synthetic polypeptide
GLVYLIYYEESLHHPMYFFFGHALSLIDLLTCTTTLPNAL

SEQ ID NO: 937 - protein, 31 aa, Artificial Sequence
  Synthetic polypeptide
MSYFPILFFFFLKRCPSYTEPQNLTGVSEFL

SEQ ID NO: 938 - protein, 40 aa, Artificial Sequence
  Synthetic polypeptide
DSANAKTLLEAASKFQFHTFCKVCVSFLEKQLTASNCLGV

SEQ ID NO: 939 - protein, 37 aa, Artificial Sequence
  Synthetic polypeptide
LWENETVGAQDDPLAYWEKKREAWPPSICLTPHRSLL

SEQ ID NO: 940 - protein, 40 aa, Artificial Sequence
  Synthetic polypeptide
SDPSQQPPSYGGPSVPGSGGPPAGGSGFGRGQNHNVQGFH

SEQ ID NO: 941 - protein, 40 aa, Artificial Sequence
  Synthetic polypeptide
PGSRPKKKLSPPSITIDPPESQGPRTPPSPGICLRRRAPS

SEQ ID NO: 942 - protein, 40 aa, Artificial Sequence
  Synthetic polypeptide
MSGQDVIKAVEDGFRLPPPRNCPNLLHRLMLDCWQKDPGE

SEQ ID NO: 943 - protein, 40 aa, Artificial Sequence
  Synthetic polypeptide
AVVGIEDPVRPEVPDAIKKCQRAGITVRMVTGDNINTARA

SEQ ID NO: 944 - protein, 40 aa, Artificial Sequence
  Synthetic polypeptide
GLPSKIGSISRQSSLSEKKIPEPSPVTRRKAYEKAEKSKA

SEQ ID NO: 945 - protein, 33 aa, Artificial Sequence
  Synthetic polypeptide
MAERESGGLGGGAASPPAASPFLGLHIASPPNF

SEQ ID NO: 946 - protein, 40 aa, Artificial Sequence
  Synthetic polypeptide
QGIAVLNIPSYAGGTNFWGGTKEDDTFAAPSFDDKILEVV

SEQ ID NO: 947 - protein, 40 aa, Artificial Sequence
  Synthetic polypeptide
APRFSRGLRGELSYNLGAGGGSAEVDTSSLSGDNTLMSTL

SEQ ID NO: 948 - protein, 40 aa, Artificial Sequence
  Synthetic polypeptide
QCYQEVCNDRINANTITSPRLAALTYKCTRDQWTVYCRVI

SEQ ID NO: 949 - protein, 19 aa, Artificial Sequence
  Synthetic polypeptide
IIIKCLLYARHGVLFLFFF

SEQ ID NO: 950 - protein, 40 aa, Artificial Sequence
  Synthetic polypeptide
RTAPLGVPGTLPGLPRRDPLRVALRLDAACWEWARSGCAR

SEQ ID NO: 951 - protein, 40 aa, Artificial Sequence
  Synthetic polypeptide
HKTLLERHVALHSASNGTPPAGTPPGARAGPPGVVACTEG

SEQ ID NO: 952 - protein, 40 aa, Artificial Sequence
  Synthetic polypeptide
RLGSVMRPTEDITARELPPPTSAQGPGRVGPRRQRKHCIT

SEQ ID NO: 953 - protein, 40 aa, Artificial Sequence
  Synthetic polypeptide
SLRRHYEVHHGLCILKEAPPEEEACGDSPHAHESAGQPPP

SEQ ID NO: 954 - protein, 40 aa, Artificial Sequence
  Synthetic polypeptide
RGACPGLLETLGALRAIPPAQLQEEAFMSQVHSVVLSERD

SEQ ID NO: 955 - protein, 37 aa, Artificial Sequence
  Synthetic polypeptide
AHLRKAEREEKPKHTEAKKSLSFRKKQQKDFCFIFRN

SEQ ID NO: 956 - protein, 40 aa, Artificial Sequence
  Synthetic polypeptide
AGQGCKDALQLLIEHSWERGERLDMQALKQSSTELLFGGH

SEQ ID NO: 957 - protein, 40 aa, Artificial Sequence
  Synthetic polypeptide
SCGPGTQHRQLQCRQEFGGGGSSVPPERCGHLPRPNITQS

SEQ ID NO: 958 - protein, 40 aa, Artificial Sequence
  Synthetic polypeptide
SSSLSSGHVHSTPFQAGTPRYDVPIDMSYDSYPHHGIGTQ

SEQ ID NO: 959 - protein, 40 aa, Artificial Sequence
  Synthetic polypeptide
ASTVEGGDTALLPEFPRGPLDAYRARASFSWKELALFTEG

SEQ ID NO: 960 - protein, 40 aa, Artificial Sequence
  Synthetic polypeptide
ARRGMHAFIVPIRSLQDHTPLPGIIIGDIGPKMDFDQTDN

SEQ ID NO: 961 - protein, 36 aa, Artificial Sequence
  Synthetic polypeptide
MVTQILGAMESQVGGGPAGPALPNGPLLGTNGATDD

SEQ ID NO: 962 - protein, 40 aa, Artificial Sequence
  Synthetic polypeptide
YGRDRGIFGIESWPDYEDIYKKTIEVGTFLDLPRFPDITE

SEQ ID NO: 963 - protein, 31 aa, Artificial Sequence
  Synthetic polypeptide
MNQENRSSFFWLLVIFTFLLKITASFSMSAY

SEQ ID NO: 964 - protein, 40 aa, Artificial Sequence
  Synthetic polypeptide
STLRGRARAMSKASKVPGGVQARLEKDAAAPALEDLPWTS

SEQ ID NO: 965 - protein, 40 aa, Artificial Sequence
  Synthetic polypeptide
LKKFKMHLEDYPPQKGCIPLPRGQTEKADHVDLATLMIDF

SEQ ID NO: 966 - protein, 40 aa, Artificial Sequence
  Synthetic polypeptide
GLKTKKWVNEVRYGGFSLGGRDPGLPSGQELGRSVEELWA

SEQ ID NO: 967 - protein, 40 aa, Artificial Sequence
  Synthetic polypeptide
GHQEGLVELPASFRELLTFFCTNATIHGAIRLVCSRGNRL

SEQ ID NO: 968 - protein, 40 aa, Artificial Sequence
  Synthetic polypeptide
GGGAEARGATAGASACQGGLYGGVAGVAYMLYHVSQSPLF

SEQ ID NO: 969 - protein, 40 aa, Artificial Sequence
  Synthetic polypeptide
DVECHLLTHVPMWSARLLTCPCGVPACSHVPMRSARLLTR

SEQ ID NO: 970 - protein, 40 aa, Artificial Sequence
  Synthetic polypeptide
EEKPGRKRAEAKGNRSWSEESLKPSDNEQGLPVFSGSPPM

SEQ ID NO: 971 - protein, 40 aa, Artificial Sequence
  Synthetic polypeptide
SLASPMRLSTPSASPAIPPLVHCADKSLPWKMGVSPGNPV

SEQ ID NO: 972 - protein, 40 aa, Artificial Sequence
  Synthetic polypeptide
LAQPPKDLTLELAGSPSVPLVIGCAVSCMALLTLLAIYAA

SEQ ID NO: 973 - protein, 40 aa, Artificial Sequence
  Synthetic polypeptide
DRNNSSCRNYNKQASEQNWANYSAEQNRMGQAGSTISNSH

SEQ ID NO: 974 - protein, 40 aa, Artificial Sequence
  Synthetic polypeptide
DGYTGEHCEVSARSGRCTPGVCKNGGTCVNLLVGGFKCDC

SEQ ID NO: 975 - protein, 40 aa, Artificial Sequence
  Synthetic polypeptide
SELKEARQRRQPPGHRPAPRGGGGSALVRGSPCVESCWAP

SEQ ID NO: 976 - protein, 40 aa, Artificial Sequence
  Synthetic polypeptide
MAAKTPSSEESGLPKLPVPPLQQTLATYLQCMRHLVSEEQ

SEQ ID NO: 977 - protein, 40 aa, Artificial Sequence
  Synthetic polypeptide
SYTVENAYECSECGKAFKKKFHFIRHEKNHTRKKPFECND

SEQ ID NO: 978 - protein, 40 aa, Artificial Sequence
  Synthetic polypeptide
TYPPSASVVGASVGGHRHPPGGGGGQRSLSPGGAALGYRD

SEQ ID NO: 979 - protein, 40 aa, Artificial Sequence
  Synthetic polypeptide
ADKPASLPPASQASNHRDPRQARRLATETGEGEGEPLSRL

SEQ ID NO: 980 - protein, 40 aa, Artificial Sequence
  Synthetic polypeptide
TAQLKTTPTQPSEQKAAFPPPEQKTAFDKKLLDRFDYDDE

SEQ ID NO: 981 - protein, 40 aa, Artificial Sequence
  Synthetic polypeptide
KKERTSIFEMSDFSCVGKKTRTVDITNFTAKTISSPRKTG

SEQ ID NO: 982 - protein, 40 aa, Artificial Sequence
  Synthetic polypeptide
LGNPFGLIREFSEGVEAFFYEPYQGAIQGPEEFVEGMALG

SEQ ID NO: 983 - protein, 40 aa, Artificial Sequence
  Synthetic polypeptide
ESRHSLEERLQQIREDEEREGSELTLNSREGAPTQHPLSL

SEQ ID NO: 984 - protein, 40 aa, Artificial Sequence
  Synthetic polypeptide
RLREEEKRRRREEERCKKKETDKQKKIAEKEVRIKLLKKP

SEQ ID NO: 985 - protein, 40 aa, Artificial Sequence
  Synthetic polypeptide
SQQPPSYGGPSVPGSGGPPAGGSGFGRGQNHNVQGFHPYR

SEQ ID NO: 986 - protein, 40 aa, Artificial Sequence
  Synthetic polypeptide
ALVVLDVHARDVLSSLVKKNISDDSDFEWLSQLRYYWQEN

SEQ ID NO: 987 - protein, 40 aa, Artificial Sequence
  Synthetic polypeptide
AYIQSRQALNSVVKITSKKKHPELITFKYGNSSASGIEIL

SEQ ID NO: 988 - protein, 30 aa, Artificial Sequence
  Synthetic polypeptide
VKVVQGPAGGDNSKLRYKKKGSHCLEVTVQ

SEQ ID NO: 989 - protein, 40 aa, Artificial Sequence
  Synthetic polypeptide
DNTWSITCPLCRKVTAVPGGLICSLRDHEAVVGQLAQPCT

SEQ ID NO: 990 - protein, 40 aa, Artificial Sequence
  Synthetic polypeptide
RWTEMIRASRENPAMQEKKRSSIWQFFSRLFSSSSNTTKK

SEQ ID NO: 991 - protein, 40 aa, Artificial Sequence
  Synthetic polypeptide
WVSQFVTFYPGDVILTGTPPGVGVFRKPPVFLKKGDEVQC

SEQ ID NO: 992 - protein, 40 aa, Artificial Sequence
  Synthetic polypeptide
AFVGLLASCLGLELSRCRAKPPGRACSNPSFLRFQLDFYQ

SEQ ID NO: 993 - protein, 40 aa, Artificial Sequence
  Synthetic polypeptide
LVARALANECSQGDKKVAFFMRKGADCLSKWVGESERQLR

SEQ ID NO: 994 - protein, 40 aa, Artificial Sequence
  Synthetic polypeptide
GVDVCFFGMHVQEYGSDCPPPNTRRVYISYLDSIHFFRPR

SEQ ID NO: 995 - protein, 40 aa, Artificial Sequence
  Synthetic polypeptide
TASALLPKRAMQFGSRIAKMEKINEKASDKCGRLQIMSLE

SEQ ID NO: 996 - protein, 40 aa, Artificial Sequence
  Synthetic polypeptide
PRGPKVGSLGLPAHPREKKTSKSSKIRSLADYRTEDSNAG

SEQ ID NO: 997 - protein, 40 aa, Artificial Sequence
  Synthetic polypeptide
GKKYQWDAETQGWILGSFFYGYIITQIPGGYVASKIGGKM

SEQ ID NO: 998 - protein, 40 aa, Artificial Sequence
  Synthetic polypeptide
ISLQDLSKERRPGGAGGPPIQDEDEGEEGPTEPPPAEPRT

SEQ ID NO: 999 - protein, 34 aa, Artificial Sequence
  Synthetic polypeptide
MAAGCLLALTLTLFQSLLIGPSSEEPFPSAVTIK

SEQ ID NO: 1000 - protein, 40 aa, Artificial Sequence
  Synthetic polypeptide
VASHPETRSAFLAAHIPLFLYPFLHTVSKTRPFEYLRLTS

SEQ ID NO: 1001 - protein, 32 aa, Artificial Sequence
  Synthetic polypeptide
CQKCGKAFSRASTLWKHKKTHTGEKPYKCKKM

SEQ ID NO: 1002 - protein, 40 aa, Artificial Sequence
  Synthetic polypeptide
WDMAQLRAVVVDDYRRRKKKGGPLRALSSKTWSTDDFFAG

SEQ ID NO: 1003 - protein, 40 aa, Artificial Sequence
  Synthetic polypeptide
IQMKSADRAFMAAQKCHKKNMKDRYVEVFQCSAEEMNFVL

SEQ ID NO: 1004 - protein, 40 aa, Artificial Sequence
  Synthetic polypeptide
PAVPFSRSRQPSPLLLLPPPAGLTSDPGPSVRRVPAVQRD

SEQ ID NO: 1005 - protein, 30 aa, Artificial Sequence
  Synthetic polypeptide
ESTQLQNEIQKLQKTLKKKTKRYMSHKLKI

SEQ ID NO: 1006 - protein, 40 aa, Artificial Sequence
  Synthetic polypeptide
GLAMSSSIFIGGSFILKKKGLLRLARKGSMRAGQGGHAYL

SEQ ID NO: 1007 - protein, 40 aa, Artificial Sequence
  Synthetic polypeptide
DLALRGKKKRKKFMKDAKKKGEMTAEERSQFEILKAQMFA

SEQ ID NO: 1008 - protein, 40 aa, Artificial Sequence
  Synthetic polypeptide
NLLPYKIAYYIEGIENSVFTLSEGHSAQICTAQLGKARLH

SEQ ID NO: 1009 - protein, 37 aa, Artificial Sequence
  Synthetic polypeptide
MKSCGVSLATAAAAAAAFGDEEKKMAAGKASGESEEA

SEQ ID NO: 1010 - protein, 40 aa, Artificial Sequence
  Synthetic polypeptide
LRDSIQSAQELLAQEQKKKEELEIATSQLKSDLTSRDDLI

SEQ ID NO: 1011 - protein, 40 aa, Artificial Sequence
  Synthetic polypeptide
PGCLPMVKRTITRQQWKKKALRSMPKSRNQVLFRRNLTPS

SEQ ID NO: 1012 - protein, 40 aa, Artificial Sequence
  Synthetic polypeptide
EASASPPRSEAQRQIQEWGVSVRTLRGNFESASGPLCGFN

SEQ ID NO: 1013 - protein, 40 aa, Artificial Sequence
  Synthetic polypeptide
TTTDMYLLILQHFLLHATPPDSASQGLGPSLLRGRLPTLL

SEQ ID NO: 1014 - protein, 40 aa, Artificial Sequence
  Synthetic polypeptide
RGKPPPQAHLPSAPALPPPHPPVVLPHLQHSVAGHHLGPP

SEQ ID NO: 1015 - protein, 20 aa, Artificial Sequence
  Synthetic polypeptide
LQLTSGVHSTIKVIKAKKKT

SEQ ID NO: 1016 - protein, 40 aa, Artificial Sequence
  Synthetic polypeptide
VRQGALQGGLLMGYSPAGGATSPGVYQVSIFSPPAGTSEP

SEQ ID NO: 1017 - protein, 23 aa, Artificial Sequence
  Synthetic polypeptide
HYSGGESHNSSSSKTFEKKRGKK

SEQ ID NO: 1018 - protein, 40 aa, Artificial Sequence
  Synthetic polypeptide
SEHHLQRAISAQQVFREKKESMVIPVPEAESNVNYYNRLY

SEQ ID NO: 1019 - protein, 40 aa, Artificial Sequence
  Synthetic polypeptide
KKRFKSLEKSHKNTGELKKSKVLSHHRAGRSNQIKIEQIK

SEQ ID NO: 1020 - protein, 40 aa, Artificial Sequence
  Synthetic polypeptide
CDECGKTFIRHDHLTKHKKIHSGEKAHQCEECGKCFGRRD

SEQ ID NO: 1021 - protein, 40 aa, Artificial Sequence
  Synthetic polypeptide
DQPSILNSCEDPVPGMLFFLPPGQHLSDYSQLNESTTKES

SEQ ID NO: 1022 - protein, 40 aa, Artificial Sequence
  Synthetic polypeptide
RRKSKRMSKYKENKSENKKTVPQKKMHKSVSSNDAYNFNL

SEQ ID NO: 1023 - protein, 40 aa, Artificial Sequence
  Synthetic polypeptide
VHDASTSSDSEEQDMSVKKGDDLLETNNPEPEKCQSVSSA

SEQ ID NO: 1024 - protein, 40 aa, Artificial Sequence
  Synthetic polypeptide
ILTSLWLLEQPYFATYKAKNAIIKMVENRDTGCQIGPNIE

SEQ ID NO: 1025 - protein, 40 aa, Artificial Sequence
  Synthetic polypeptide
KEQDRVHSPCPTSGSEKKKRSDDPVEDDKEKKELGYLTVE

SEQ ID NO: 1026 - protein, 40 aa, Artificial Sequence
  Synthetic polypeptide
VPTIFSLPEDNQGKDPSKKKSQKKNLEDEKEVCPKAKSEE

SEQ ID NO: 1027 - protein, 40 aa, Artificial Sequence
  Synthetic polypeptide
ELVEMCNGKNGILEDSQKKEDTAFSDWSDEDVPDRTEVTE

SEQ ID NO: 1028 - protein, 40 aa, Artificial Sequence
  Synthetic polypeptide
EPQKSGNNETFTPNRVEKKKLQHTYLCEEKENNKSFQSDD

SEQ ID NO: 1029 - protein, 34 aa, Artificial Sequence
  Synthetic polypeptide
LAGGRHCCPVCRWPSYKKKQPYAQHQPLSNDVPS

SEQ ID NO: 1030 - protein, 40 aa, Artificial Sequence
  Synthetic polypeptide
KQNVKMSESQAALPSALKTLQQKLRLHIIEIIGNEGLLAC

SEQ ID NO: 1031 - protein, 20 aa, Artificial Sequence
  Synthetic polypeptide
RPAERITEDHEKKSKRIKKN

SEQ ID NO: 1032 - protein, 40 aa, Artificial Sequence
  Synthetic polypeptide
NYKVDCACHKGNRNCPIQKRNPNATELPLLPPPPSLPTIG

SEQ ID NO: 1033 - protein, 32 aa, Artificial Sequence
  Synthetic polypeptide
NGSFYFSGICYLLSSVSFFFVPLAERWKNSLT

SEQ ID NO: 1034 - protein, 40 aa, Artificial Sequence
  Synthetic polypeptide
VIEVGKKHGPWVNHYSIFFVSVSFFIITAATVGYFIFYSA

SEQ ID NO: 1035 - protein, 40 aa, Artificial Sequence
  Synthetic polypeptide
PDEIGNFIDENLKAADTDPTAPPYDSLLVFDYEGSGSEAA

SEQ ID NO: 1036 - protein, 40 aa, Artificial Sequence
  Synthetic polypeptide
TRWKEDIRYHYAEISSQVPLGKRLREYFNSEKPEGRIIMT

SEQ ID NO: 1037 - protein, 40 aa, Artificial Sequence
  Synthetic polypeptide
VLDKVFRASESQILSIAEKMLDTRVAENRDLGMNENNIFE

SEQ ID NO: 1038 - protein, 40 aa, Artificial Sequence
  Synthetic polypeptide
YRTSMFKTFKKTLDDGFFPFIILDAINDRVRHFDQFWSAA

SEQ ID NO: 1039 - protein, 40 aa, Artificial Sequence
  Synthetic polypeptide
KEKYQEEFEHFQQELDKKKEEFQKGHPDLQGQPAEEIFES

SEQ ID NO: 1040 - protein, 40 aa, Artificial Sequence
  Synthetic polypeptide
SLNRITDIGEVSQFLTEGIIMKDFSHPNVLSLLGICLRSE

SEQ ID NO: 1041 - protein, 40 aa, Artificial Sequence
  Synthetic polypeptide
ASSSSSSSSSSSRSRSRSLSPPHKRWRRSSCSSSGRSRRC

SEQ ID NO: 1042 - protein, 40 aa, Artificial Sequence
  Synthetic polypeptide
ERQMAELMPVGDNNFSDSEEGEGLEESADIKGEPHGLENM

SEQ ID NO: 1043 - protein, 40 aa, Artificial Sequence
  Synthetic polypeptide
DPKPALRWGDSKGSNCQGGWEDDSAATGMVKSNQWGNCKE

SEQ ID NO: 1044 - protein, 40 aa, Artificial Sequence
  Synthetic polypeptide
KPQIKQVVPEFVNASADAGGSSATYMDQAPSPAVCPQAPY

SEQ ID NO: 1045 - protein, 40 aa, Artificial Sequence
  Synthetic polypeptide
KWCQRKLQAELKIGSFRFFWIQNVSLKFQQHQQTVEIDNL

SEQ ID NO: 1046 - protein, 40 aa, Artificial Sequence
  Synthetic polypeptide
DPNFGSKEDFDSLLQSAKKKSIRVILDLTPNYRGENSWFS

SEQ ID NO: 1047 - protein, 40 aa, Artificial Sequence
  Synthetic polypeptide
SILYYLGLVQWVVQKVAWFLQITMGTTATETLAVAGNIFV

SEQ ID NO: 1048 - protein, 40 aa, Artificial Sequence
  Synthetic polypeptide
SNVNRSLSGDQDTFHPSGGSHTTHGSESDGHSHGSQEGGA

SEQ ID NO: 1049 - protein, 32 aa, Artificial Sequence
  Synthetic polypeptide
RKEEREIKDEKYIDNLEKKQWITKWNENESYS

SEQ ID NO: 1050 - protein, 40 aa, Artificial Sequence
  Synthetic polypeptide
ELSPLLMILSQLLPQQRHGERTPYVLRCLTEVALCQDKRS

SEQ ID NO: 1051 - protein, 40 aa, Artificial Sequence
  Synthetic polypeptide
QAAAVQKRVETVSQTLRKKNKQYQIPDVRDIFAQQRESKE

SEQ ID NO: 1052 - protein, 40 aa, Artificial Sequence
  Synthetic polypeptide
KLHCKQDGEEGTEEDTEEKCTICLSILEEGEDVRRLPCMH

SEQ ID NO: 1053 - protein, 40 aa, Artificial Sequence
  Synthetic polypeptide
MTELEPSKFSKQAAENEKKYYIEKLFERYGENGRLSFFGL

SEQ ID NO: 1054 - protein, 40 aa, Artificial Sequence
  Synthetic polypeptide
SSPSPGQQVQTPQSMPPPPQPSPQPGQPSSQPNSNVSSGP

SEQ ID NO: 1055 - protein, 40 aa, Artificial Sequence
  Synthetic polypeptide
KPDAEYPEWLFEMNLGPPKTLEELDPESREYWRRLRKQNI

SEQ ID NO: 1056 - protein, 40 aa, Artificial Sequence
  Synthetic polypeptide
QMVAFLEQRASALLASCSKNCTNSPAIVRFSGQSRGVPAV

SEQ ID NO: 1057 - protein, 40 aa, Artificial Sequence
  Synthetic polypeptide
WWFWPLCCKVVIKDPPPPPAPAPKEEEEEPLPTKKWPTVD

SEQ ID NO: 1058 - protein, 40 aa, Artificial Sequence
  Synthetic polypeptide
IKDKLKCYDFDVHTMKTLKNIISPPWDFREFEVEKQTAEE

SEQ ID NO: 1059 - protein, 40 aa, Artificial Sequence
  Synthetic polypeptide
IHTGEKPYECSDCGKSFTKKSQLQVHQRIHTGEKPYVCAE

SEQ ID NO: 1060 - protein, 40 aa, Artificial Sequence
  Synthetic polypeptide
WRKLVSKTQLEMNLPLMIKKQDQPTFDNSGNILSKEEKAT

SEQ ID NO: 1061 - protein, 40 aa, Artificial Sequence
  Synthetic polypeptide
RQRELQLSVLSAESLRENFFLGGVTLPLKDFNLSKETVKW

SEQ ID NO: 1062 - protein, 40 aa, Artificial Sequence
  Synthetic polypeptide
ESHNFSGDIALLELQHSIPLGPNVLPVCLPDNETLYRSGL

SEQ ID NO: 1063 - protein, 31 aa, Artificial Sequence
  Synthetic polypeptide
LSPVDCIPEENNSAHPSFFSSSSKGDSFAQH

SEQ ID NO: 1064 - protein, 40 aa, Artificial Sequence
  Synthetic polypeptide
LKIEETNPSLAQDTVIIKKKSCSSKALNTPVLSVLKEAAK

SEQ ID NO: 1065 - protein, 40 aa, Artificial Sequence
  Synthetic polypeptide
AIARLDNSAAKHKLAVKPKKQRVSKKHRRLAQDPQHEQGG

SEQ ID NO: 1066 - protein, 40 aa, Artificial Sequence
  Synthetic polypeptide
DAQRFMDLATYINMIWSAPLQVILALYLLWLNLGPSVLAG

SEQ ID NO: 1067 - protein, 40 aa, Artificial Sequence
  Synthetic polypeptide
AAVCPTDLPQLWKGEGAPGQPAEDSVKQEGLDLTGTAATA

SEQ ID NO: 1068 - protein, 40 aa, Artificial Sequence
  Synthetic polypeptide
LALSVETDYTFPLAEKVKAFLADPSAFVAAAPVAAATTAA

SEQ ID NO: 1069 - protein, 40 aa, Artificial Sequence
  Synthetic polypeptide
QKYQKKEKKKEKKSKSKKGKHHKKEKKKRKKEKHSSTPNS

SEQ ID NO: 1070 - protein, 40 aa, Artificial Sequence
  Synthetic polypeptide
TVFLTAILGGTIVIVIGFFAVLLCYCRDKCGTPQKRERNI

SEQ ID NO: 1071 - protein, 40 aa, Artificial Sequence
  Synthetic polypeptide
LSAQMLAPPPPGLPRLALPPATKPATTSEGGATSPTSPSY

SEQ ID NO: 1072 - protein, 40 aa, Artificial Sequence
  Synthetic polypeptide
LNFGTVSFYLGYNAMQDFFPTMSMKPNPQCDDRNCRKQQE

SEQ ID NO: 1073 - protein, 40 aa, Artificial Sequence
  Synthetic polypeptide
VLVHKKKDCPPGSFWWLIPLLLLLLPLLALLLLLCWKYCA

SEQ ID NO: 1074 - protein, 40 aa, Artificial Sequence
  Synthetic polypeptide
PNLMPSNPDSGMYSPSRYPPQQQQQQQQRHDSYGNQFSTQ

SEQ ID NO: 1075 - protein, 40 aa, Artificial Sequence
  Synthetic polypeptide
LCNVTLNSAQQAQAHYQGKNHGKKLRNYYAANSCPPPARM

SEQ ID NO: 1076 - protein, 35 aa, Artificial Sequence
  Synthetic polypeptide
VQKAEALMRELDEEGSDPPLPGRAQRIRQVLQLLS

SEQ ID NO: 1077 - protein, 40 aa, Artificial Sequence
  Synthetic polypeptide
SDLSSQFVISPPALRSRQKNTSNKNKLEDELKDDAQSVET

SEQ ID NO: 1078 - protein, 40 aa, Artificial Sequence
  Synthetic polypeptide
NSPTWSLQVFSKKKKKKKKNNMAAKEKLEAVLNVALRVPS

SEQ ID NO: 1079 - protein, 40 aa, Artificial Sequence
  Synthetic polypeptide
YLESSLISHESAVTALVPPGSESFDILTAGIQATSPLTTV

SEQ ID NO: 1080 - protein, 40 aa, Artificial Sequence
  Synthetic polypeptide
ERNRKRSRSRSSSSGDRKKRRTRSRSPERRHRSSSGSSHS

SEQ ID NO: 1081 - protein, 40 aa, Artificial Sequence
  Synthetic polypeptide
SGKRRFLLCLLLFTVITYFFVVIGIAPIFILYELDSPLCW

SEQ ID NO: 1082 - protein, 40 aa, Artificial Sequence
  Synthetic polypeptide
LQTRADYLIKLLSRDLAKKEALSGAGSSKRRKARAKKNKA

SEQ ID NO: 1083 - protein, 40 aa, Artificial Sequence
  Synthetic polypeptide
SSEIKVKVEPADSVESSPPSITHSPQNELKGTNHSNEKKN

SEQ ID NO: 1084 - protein, 40 aa, Artificial Sequence
  Synthetic polypeptide
VSPLPCCTQGHDCQHFYPPSDFTVSTQVFRDMKRSHSLQK

SEQ ID NO: 1085 - protein, 30 aa, Artificial Sequence
  Synthetic polypeptide
HVSQAQQETYLGFWINSKKSQCNIFLSGTY

SEQ ID NO: 1086 - protein, 40 aa, Artificial Sequence
  Synthetic polypeptide
IWGTDVLKNRSVTGVATKKKKDAVPKPPLSPHKLSIVREC

SEQ ID NO: 1087 - protein, 40 aa, Artificial Sequence
  Synthetic polypeptide
IRQHPGHAHYHLPAAYLLGPSRSAVARPPRPGPFLPSQEP

SEQ ID NO: 1088 - protein, 40 aa, Artificial Sequence
  Synthetic polypeptide
KHMSLSYVANQEPGILQQKNAVQIISSALDTDNESTKDTE

SEQ ID NO: 1089 - protein, 40 aa, Artificial Sequence
  Synthetic polypeptide
QIMLDMLTENLFFDTGMGKSKFLQDMHTLLLTRHRDEHEG

SEQ ID NO: 1090 - protein, 40 aa, Artificial Sequence
  Synthetic polypeptide
KVTLLQLLLGHKNEENVEKNTSPQGVHNDVSKFNTQNYAR

SEQ ID NO: 1091 - protein, 40 aa, Artificial Sequence
  Synthetic polypeptide
SFTQLSEEIQMAVVWCRSKKLKAQAIFLGNKLLKSNRLKH

SEQ ID NO: 1092 - protein, 40 aa, Artificial Sequence
  Synthetic polypeptide
PPRQPFLPGPGQPFLPTHTQPNLQGPLHPPLPPPHQPQPQ

SEQ ID NO: 1093 - protein, 40 aa, Artificial Sequence
  Synthetic polypeptide
TISLVAVSDVNKHADRIAFWDDVYGFKMSCMKKAVIPEAV

SEQ ID NO: 1094 - protein, 40 aa, Artificial Sequence
  Synthetic polypeptide
LKTVGKLTATQVAKISFFFCFVWFLANLSYQEALSDTQVA

SEQ ID NO: 1095 - protein, 40 aa, Artificial Sequence
  Synthetic polypeptide
YLRKDEGSNKQVYSVPHFFLAGAAKERSQMNSQTEDHALA

SEQ ID NO: 1096 - protein, 40 aa, Artificial Sequence
  Synthetic polypeptide
GESVLRSVSPVQDLDDDTPPSPAHSDMPYDARQNPNCKPP

SEQ ID NO: 1097 - protein, 40 aa, Artificial Sequence
  Synthetic polypeptide
VIQSQSNSFHAKRAEQLKKSILKQAADLTQELPSVLLLHQ

SEQ ID NO: 1098 - protein, 40 aa, Artificial Sequence
  Synthetic polypeptide
VSNDLKYDAERDLRDIGAKNILVHSLNKFKYGKISSKHNG

SEQ ID NO: 1099 - protein, 40 aa, Artificial Sequence
  Synthetic polypeptide
ARSSRVIKTPRRFMDEDPPKPPKVEVSPVLRPPITTSPPV

SEQ ID NO: 1100 - protein, 40 aa, Artificial Sequence
  Synthetic polypeptide
PRSFLAKKLQLVRRVGAPPRRMASPPAPSPAPPAISPIIK

SEQ ID NO: 1101 - protein, 40 aa, Artificial Sequence
  Synthetic polypeptide
NGHIHFDNVSVVSLQDGKKEPSSCTCLKGPKLSEIGTIAW

SEQ ID NO: 1102 - protein, 40 aa, Artificial Sequence
  Synthetic polypeptide
WAIKLATNAAVTVLRVDQIIMAKPAGGPKPPSGKKDWDDD

SEQ ID NO: 1103 - protein, 40 aa, Artificial Sequence
  Synthetic polypeptide
ARCPSARGSGDGEMGKPRNVALITGITGQDGSYLAEFLLE

SEQ ID NO: 1104 - protein, 40 aa, Artificial Sequence
  Synthetic polypeptide
KGGWQQKSKGPKKTAKSKKKKPLKKKPTPVLLPQSKQQKQ

SEQ ID NO: 1105 - protein, 40 aa, Artificial Sequence
  Synthetic polypeptide
FLLGRWCYQVSHLSWLEKKTATALLESPLSATVEDALQSF

SEQ ID NO: 1106 - protein, 40 aa, Artificial Sequence
  Synthetic polypeptide
DSLKNVIARAISKLPIVHFCSSKPRVEYSTKIVEVFCGKE

SEQ ID NO: 1107 - protein, 40 aa, Artificial Sequence
  Synthetic polypeptide
LPTMPPPVLPPSLPPPVMPPALPATVPPPGMPPPVMPPSL

SEQ ID NO: 1108 - protein, 23 aa, Artificial Sequence
  Synthetic polypeptide
DSEVADSPSSDERRIIETPPHRY

SEQ ID NO: 1109 - protein, 27 aa, Artificial Sequence
  Synthetic polypeptide
ILDCNSVRQSIMSVCFFFFLLYSQHDV

SEQ ID NO: 1110 - protein, 28 aa, Artificial Sequence
  Synthetic polypeptide
IILDCNSVRQSIMSVCFFFFLLYSQHDV

SEQ ID NO: 1111 - protein, 40 aa, Artificial Sequence
  Synthetic polypeptide
HERRVFHLTVAEPHAEPPPRGSPGNGSSHSGAPGPDPTLA

SEQ ID NO: 1112 - protein, 40 aa, Artificial Sequence
  Synthetic polypeptide
DLEPHSFGGLLEGIRGASGGAGGRSLDSRLELASLGLGAP

SEQ ID NO: 1113 - protein, 40 aa, Artificial Sequence
  Synthetic polypeptide
GAGQHPQPQPPLHKANQPPHGVPQLSLYEHFNSPHPTPAP

SEQ ID NO: 1114 - protein, 40 aa, Artificial Sequence
  Synthetic polypeptide
DISQDNALRDEMRALAGNPKATPPQIVNGDQYCGDYELFV

SEQ ID NO: 1115 - protein, 40 aa, Artificial Sequence
  Synthetic polypeptide
KSGRYMELEQRYMDLAENARFEREQLLGVQQHLSNTLKMA

SEQ ID NO: 1116 - protein, 40 aa, Artificial Sequence
  Synthetic polypeptide
DLIVRCEAGEGECRTFMPPRVTHPDPTERKWAEAVVRPPG

SEQ ID NO: 1117 - protein, 40 aa, Artificial Sequence
  Synthetic polypeptide
EKYSNLVQSVLSSRGVAQTPGSVEEDALLCGPVSKHKLPN

SEQ ID NO: 1118 - protein, 40 aa, Artificial Sequence
  Synthetic polypeptide
KAKEAAALGSRGSCSTEVEKETQEKMTILQTYFRQNRDEV

SEQ ID NO: 1119 - protein, 40 aa, Artificial Sequence
  Synthetic polypeptide
GGWQQKSKGPKKTAKSKKKKPLKKKPTPVLLPQSKQQKQK

SEQ ID NO: 1120 - protein, 40 aa, Artificial Sequence
  Synthetic polypeptide
QIVSASVLQNKFSPPSPLPQAVFSTSSRFWSSPPLLGQQP

SEQ ID NO: 1121 - protein, 40 aa, Artificial Sequence
  Synthetic polypeptide
LQRANRTGGLYSCDITARGPCTRIEFDNDADPTSESKEDQ

SEQ ID NO: 1122 - protein, 40 aa, Artificial Sequence
  Synthetic polypeptide
VEHHTYHIKNYIMNKDLLRRVLVLMNSKHTFLALCALRFM

SEQ ID NO: 1123 - protein, 40 aa, Artificial Sequence
  Synthetic polypeptide
PITTYPPYVPEYSSGLFPPSSLLGGSPTGFGCKSRPKARS

SEQ ID NO: 1124 - protein, 40 aa, Artificial Sequence
  Synthetic polypeptide
ITALLGSLNSCCNPWIYMFFSGHLLQDCVQSFPCCQNMKE

SEQ ID NO: 1125 - protein, 40 aa, Artificial Sequence
  Synthetic polypeptide
PHHDDINFYSERKQNRPFFFACVPADSLEVIPKTIRWTIP

SEQ ID NO: 1126 - protein, 40 aa, Artificial Sequence
  Synthetic polypeptide
ARQDLGPSYNGWQVLDATPQEESEGVFRCGPASVTAIREG

SEQ ID NO: 1127 - protein, 40 aa, Artificial Sequence
  Synthetic polypeptide
LPPGPPSIFPDCPRECYCPPDFPSALYCDSRNLRKVPVIP

SEQ ID NO: 1128 - protein, 40 aa, Artificial Sequence
  Synthetic polypeptide
WFGKNCSEPYCPLGCSSRGVCVDGQCICDSEYSGDDCSEL

SEQ ID NO: 1129 - protein, 40 aa, Artificial Sequence
  Synthetic polypeptide
RRGGDHVALQPLRSEGGPPTPHRSIFAPHALPNRNGSLSY

SEQ ID NO: 1130 - protein, 40 aa, Artificial Sequence
  Synthetic polypeptide
WVGRWFRPRKGTLGAMDLGGASTQITFETTSPAEDRASEV

SEQ ID NO: 1131 - protein, 40 aa, Artificial Sequence
  Synthetic polypeptide
TADEPMVFVDDQLPCNVTFFNASHVVCQTRDLAPGPHYLS

SEQ ID NO: 1132 - protein, 40 aa, Artificial Sequence
  Synthetic polypeptide
CLKKGDFSLYPTSVHYQTPLGYERITTFDSSGNVEEVCRP

SEQ ID NO: 1133 - protein, 40 aa, Artificial Sequence
  Synthetic polypeptide
RAGRGGLGPPAGVANSLPPQLFAAVSRGCCTSLTHLDASR

SEQ ID NO: 1134 - protein, 40 aa, Artificial Sequence
  Synthetic polypeptide
PDPLGPRSQPACQVAHDPPRACPLCSQGTKTLSGSIAPMD

SEQ ID NO: 1135 - protein, 40 aa, Artificial Sequence
  Synthetic polypeptide
GQSFSTDAAGSRGGSDGTPRGSPSPASVSSGRKSPHSKSP

SEQ ID NO: 1136 - protein, 40 aa, Artificial Sequence
  Synthetic polypeptide
QGRRGPPGAPGEMGPQGPPGEPGFRGAPGKAGPQGRGGVS

SEQ ID NO: 1137 - protein, 40 aa, Artificial Sequence
  Synthetic polypeptide
KQRALPSLDIVVWSELPPGAGLGSSAAYSVCLAAALLTVC

SEQ ID NO: 1138 - protein, 40 aa, Artificial Sequence
  Synthetic polypeptide
GPPGGAGEGGPPAQAPPPPQQPPTAPPSGLKKYEEPLQSM

SEQ ID NO: 1139 - protein, 40 aa, Artificial Sequence
  Synthetic polypeptide
HFIKPLLLSEVLAWEGPFPLSMEILEVPEGRPIFLSPWVG

SEQ ID NO: 1140 - protein, 40 aa, Artificial Sequence
  Synthetic polypeptide
YFKKLVLNKAILLQVIAKKDDKYTVNIQSVEASENIDVIS

SEQ ID NO: 1141 - protein, 40 aa, Artificial Sequence
  Synthetic polypeptide
ARLQTEACRLGQLHPAAPGGLAKVQEAWATLQAKAQERGQ

SEQ ID NO: 1142 - protein, 40 aa, Artificial Sequence
  Synthetic polypeptide
VGGEGAEEQPPGAERTFCLSLPDVELSPSGGNHAEYQVAE

SEQ ID NO: 1143 - protein, 40 aa, Artificial Sequence
  Synthetic polypeptide
ASPTGDMAVGSPLMQEVGSPKDPGKSLPPVPPMGLPPPQE

SEQ ID NO: 1144 - protein, 40 aa, Artificial Sequence
  Synthetic polypeptide
SEEAATWRGRFGPSLVRGLLAVSLAANALFTSVFLYQSLR

SEQ ID NO: 1145 - protein, 31 aa, Artificial Sequence
  Synthetic polypeptide
RILIHGLQGASEPPPPLPPLAGVLPRAAQPR

SEQ ID NO: 1146 - protein, 40 aa, Artificial Sequence
  Synthetic polypeptide
RFLQLGAKVPKGALLLGPPGCGKTLLAKAVATEAQVPFLA

SEQ ID NO: 1147 - protein, 40 aa, Artificial Sequence
  Synthetic polypeptide
CPQDQSPDRVGTEMEQVSKNEGCQAGAELEELSKKAGPEE

SEQ ID NO: 1148 - protein, 40 aa, Artificial Sequence
  Synthetic polypeptide
PTATGVQPESSASIVTSYPPPSYNPTCTAYTAPSYPNYDA

SEQ ID NO: 1149 - protein, 40 aa, Artificial Sequence
  Synthetic polypeptide
SNGGTCYDSGDTFRCACPPGWKGSTCAVAKNSSCLPNPCV

SEQ ID NO: 1150 - protein, 40 aa, Artificial Sequence
  Synthetic polypeptide
GATVTLRCVGNGSVEWDGPPSPHWTLYSDGSSSILSTNNA

SEQ ID NO: 1151 - protein, 40 aa, Artificial Sequence
  Synthetic polypeptide
SEDLAPSLGETWKDESVPQVPAEGVDDTSSSEGSTVDCLD

SEQ ID NO: 1152 - protein, 40 aa, Artificial Sequence
  Synthetic polypeptide
IQATNASGSPTSMLVVDAPQCPQAPINSQCVNTSQAVQDP

SEQ ID NO: 1153 - protein, 40 aa, Artificial Sequence
  Synthetic polypeptide
RAFSPKFGELVAEEARRKGELRYMHSRVVANSEEIAFYGG

SEQ ID NO: 1154 - protein, 40 aa, Artificial Sequence
  Synthetic polypeptide
APAAQTPLLGRFLGVGAPSPAISLRNFGRVRGTPRPPHLL

SEQ ID NO: 1155 - protein, 40 aa, Artificial Sequence
  Synthetic polypeptide
NVFEPKPSVPEYKVASVGGSRCLLLHYSVSKAIWDGLILL

SEQ ID NO: 1156 - protein, 40 aa, Artificial Sequence
  Synthetic polypeptide
METCRRLIKGSADRNSPSPSSVASSDSGSTDEIQDEFERE

SEQ ID NO: 1157 - protein, 40 aa, Artificial Sequence
  Synthetic polypeptide
FFRLIKIKIIVKDTNDNAPMFPSPVINISIPENTLINSRF

SEQ ID NO: 1158 - protein, 40 aa, Artificial Sequence
  Synthetic polypeptide
LQPHHLPPPPLPPPPVMPGGGYGDWQPPPPPMPPPPGPAL

SEQ ID NO: 1159 - protein, 40 aa, Artificial Sequence
  Synthetic polypeptide
KVRLEGRSTTSLSVSWSIPPPQQSRVWKYEVTYRKKGDSN

SEQ ID NO: 1160 - protein, 40 aa, Artificial Sequence
  Synthetic polypeptide
AEEDEDLEGPPSYKPPTPKAKLEAQEMPSQLFTLGASEHS

SEQ ID NO: 1161 - protein, 40 aa, Artificial Sequence
  Synthetic polypeptide
GERDTLAGQTVDLQGEVDSLSKERELLQKAREELRQQLEV

SEQ ID NO: 1162 - protein, 40 aa, Artificial Sequence
  Synthetic polypeptide
TTDERGPPGEQGPPGPPGPPGVPGIDGIDGDRGPKGPPGP

SEQ ID NO: 1163 - protein, 40 aa, Artificial Sequence
  Synthetic polypeptide
RQPPPFPPNPMGPAFNMPPQGPGYPPPGNMNFPSQPFNQP

SEQ ID NO: 1164 - protein, 40 aa, Artificial Sequence
  Synthetic polypeptide
CKPERDGAESDASSCDPPPAREPPTSPGAAPSPLRLHRAR

SEQ ID NO: 1165 - protein, 40 aa, Artificial Sequence
  Synthetic polypeptide
KHKTTPLPPPRLADVAPTPPKTPARKRGEEGTERMVQALT

SEQ ID NO: 1166 - protein, 40 aa, Artificial Sequence
  Synthetic polypeptide
PSSEACGEAQRLPSAPSGGAPIRDMGHPQGSKQLPSTGGH

SEQ ID NO: 1167 - protein, 40 aa, Artificial Sequence
  Synthetic polypeptide
TDGSPHCVFWDHSLFQGRGGWSKEGCQAQVASASPTAQCL

SEQ ID NO: 1168 - protein, 40 aa, Artificial Sequence
  Synthetic polypeptide
SGGGGTAGARGGGGGTAAPQELNNSRPARQVRRLEFNQAM

SEQ ID NO: 1169 - protein, 40 aa, Artificial Sequence
  Synthetic polypeptide
ERQPPALKAYPAASTPAAPSPVGSSSPPLAHEAEAGAAPL

SEQ ID NO: 1170 - protein, 40 aa, Artificial Sequence
  Synthetic polypeptide
PKRYKANYCSGQCEYMFMQKYPHTHLVQQANPRGSAGPCC

SEQ ID NO: 1171 - protein, 40 aa, Artificial Sequence
  Synthetic polypeptide
GLDELDGVKAACPCPQSSPPEQKEAEPEKRPKKVSQIRIR

SEQ ID NO: 1172 - protein, 35 aa, Artificial Sequence
  Synthetic polypeptide
MTSLFRRSSSGSGGGGTAGARGGGGGTAAPQELNN

SEQ ID NO: 1173 - protein, 40 aa, Artificial Sequence
  Synthetic polypeptide
PPPPQKRYTAAGAGAGGTPDYDPHAHGLQGNGSYGTPHIQ

SEQ ID NO: 1174 - protein, 40 aa, Artificial Sequence
  Synthetic polypeptide
LMNLSAHLNDPQPIEMTVKKTLSNFRRTHHDNWQEHKQQF

SEQ ID NO: 1175 - protein, 40 aa, Artificial Sequence
  Synthetic polypeptide
TPANSRTLTRAASLRGGVGAPGSPSTPPTRFFTEKKIPHE

SEQ ID NO: 1176 - protein, 40 aa, Artificial Sequence
  Synthetic polypeptide
ELWLRLRGKGLAMLHVTRGVWGSRVRVWPLLPALLGPPRA

SEQ ID NO: 1177 - protein, 40 aa, Artificial Sequence
  Synthetic polypeptide
RYIRELQYNHTGTQFFEIKKSRPLTGLMDLAKEMTKEALP

SEQ ID NO: 1178 - protein, 40 aa, Artificial Sequence
  Synthetic polypeptide
GSQYGMHPDQRLLPGPSLGLAAAGADDLQGSVEAQCGLVL

SEQ ID NO: 1179 - protein, 40 aa, Artificial Sequence
  Synthetic polypeptide
VCSSPDYLREPKYYPGGPPTPRPLLPTRPPASPPDKAFST

SEQ ID NO: 1180 - protein, 40 aa, Artificial Sequence
  Synthetic polypeptide
TPQKNGRVQEKVMEHLLKLFGTFGVISSVRILKPGRELPP

SEQ ID NO: 1181 - protein, 40 aa, Artificial Sequence
  Synthetic polypeptide
HCSNVCSNDPPCFSVMIPPNDSRARSGARCMFFVRSSPVC

SEQ ID NO: 1182 - protein, 40 aa, Artificial Sequence
  Synthetic polypeptide
EVRLRRNASSAGRLQGLAGGAPGQKECRPFEVYLPSGKMR

SEQ ID NO: 1183 - protein, 40 aa, Artificial Sequence
  Synthetic polypeptide
NPSIRAITLGHGHILVGTKNGEILEIDKSGPMTLLVQGHM

SEQ ID NO: 1184 - protein, 40 aa, Artificial Sequence
  Synthetic polypeptide
NESYQQSCGTYLRVRQPPPRPFLDMGEGTKNRIITAEGII

SEQ ID NO: 1185 - protein, 40 aa, Artificial Sequence
  Synthetic polypeptide
ASSLSRPWEKTDKGATYTPQAPKKLTPTEKGRCASLEEIL

SEQ ID NO: 1186 - protein, 26 aa, Artificial Sequence
  Synthetic polypeptide
MDDVPAPTPAPAPPAAAAPRVPFHCS

SEQ ID NO: 1187 - protein, 40 aa, Artificial Sequence
  Synthetic polypeptide
KLRDSMEQAVLDSMGSGKKGQDVGAPNGALSRLDQLSGAQ

SEQ ID NO: 1188 - protein, 40 aa, Artificial Sequence
  Synthetic polypeptide
PLKMNPNILSQDSQHVNLFFDKNDENVILQKTTNESMENS

SEQ ID NO: 1189 - protein, 40 aa, Artificial Sequence
  Synthetic polypeptide
NLEPGFISIVKLESPRRAPRPCLSLASKARMAGERGASAV

SEQ ID NO: 1190 - protein, 40 aa, Artificial Sequence
  Synthetic polypeptide
GTVYHDMGNINLTFFTTKKKYDRMENLKLIVRALNAVQPQ

SEQ ID NO: 1191 - protein, 34 aa, Artificial Sequence
  Synthetic polypeptide
MKITNGRHGDSAGAEGTMENFTALFGAQADPPPP

SEQ ID NO: 1192 - protein, 40 aa, Artificial Sequence
  Synthetic polypeptide
LKEQLHQKDQKILLLLEEKEMIFRDMAECSTPLPEDCSPT

SEQ ID NO: 1193 - protein, 40 aa, Artificial Sequence
  Synthetic polypeptide
RFQELKAQRESKEALEIEKNSRKPPPYKHIKANKVIGKVQ

SEQ ID NO: 1194 - protein, 40 aa, Artificial Sequence
  Synthetic polypeptide
KEYLQEKAKEKYQEWLKKKNAEECERKKKEKEKEKQQQAE

SEQ ID NO: 1195 - protein, 40 aa, Artificial Sequence
  Synthetic polypeptide
TAKGGVGKLVTLRNVSTKKIPTVNRITPKTQGTNQIQKNT

SEQ ID NO: 1196 - protein, 40 aa, Artificial Sequence
  Synthetic polypeptide
GSVLKNGSLTNHFSFEKKKARVAVLISGTGSNLQALIDST

SEQ ID NO: 1197 - protein, 40 aa, Artificial Sequence
  Synthetic polypeptide
KRSQENEWVKSDQVKKRKKKRKDYQPNYFLSIPITNKEII

SEQ ID NO: 1198 - protein, 40 aa, Artificial Sequence
  Synthetic polypeptide
PGGLEGRLQATGQARPPAPRPFHHGQYYGYLSSSSPGEVE

SEQ ID NO: 1199 - protein, 40 aa, Artificial Sequence
  Synthetic polypeptide
WWLTGSNLTLSVNNSGLFFLCGNGVYKGFPPKWSGRCGLG

SEQ ID NO: 1200 - protein, 40 aa, Artificial Sequence
  Synthetic polypeptide
MILSSYFINFIYLAKSTKKTMLTLTLVCAITFLLVCSGTF

SEQ ID NO: 1201 - protein, 40 aa, Artificial Sequence
  Synthetic polypeptide
KVRYVVSKASVQTQPAIKKDASAQQDSYEFVSPSPPADVS

SEQ ID NO: 1202 - protein, 40 aa, Artificial Sequence
  Synthetic polypeptide
KTFRRSSHLTAHQSIHADKKPYECKECGKAFKMYGYLTQH

SEQ ID NO: 1203 - protein, 40 aa, Artificial Sequence
  Synthetic polypeptide
FATTLWGVHSAQTEKEKKKESSNCGRRNVFSYGRVKLCST

SEQ ID NO: 1204 - protein, 40 aa, Artificial Sequence
  Synthetic polypeptide
SVFGKEVTLETLVKDLKKKIPSLSFSPLKPNGRISVEGSF

SEQ ID NO: 1205 - protein, 40 aa, Artificial Sequence
  Synthetic polypeptide
ETADRELLPTFHHVSVYPKKELPLFIHFTAGFCSSTAMIA

SEQ ID NO: 1206 - protein, 40 aa, Artificial Sequence
  Synthetic polypeptide
ASINKKLGLLSYKDRIRKKESEVLCSTTETLEEKNENMKL

SEQ ID NO: 1207 - protein, 40 aa, Artificial Sequence
  Synthetic polypeptide
DTVGTLSLIMLAQAQEVFFLKATRDKMKDAIIAKLANQAA

SEQ ID NO: 1208 - protein, 40 aa, Artificial Sequence
  Synthetic polypeptide
LDDLITPAKLSVGFELLRKMGWKEGQGVGPRVKRRPRRQK

SEQ ID NO: 1209 - protein, 40 aa, Artificial Sequence
  Synthetic polypeptide
PDEVCYRVLMQLCSHYGQPVLSVRVMLEMRQAGIVPNTIT

SEQ ID NO: 1210 - protein, 40 aa, Artificial Sequence
  Synthetic polypeptide
TEDIKSAFAPFGKISDARVVKDMATGKSKGYGFVSFYNKL

SEQ ID NO: 1211 - protein, 40 aa, Artificial Sequence
  Synthetic polypeptide
SIINTLQTQVEVKKRRHRLKRHNDCFVGSEAVDVIFSHLI

SEQ ID NO: 1212 - protein, 40 aa, Artificial Sequence
  Synthetic polypeptide
SDNFNTGNMTVLSPYLNTTVLPSSSSSRGSLDSSRSEKDR

SEQ ID NO: 1213 - protein, 40 aa, Artificial Sequence
  Synthetic polypeptide
SLLHTAGGGSHGQLGSGSSSEASSVPHLLAQPSVSLGDQP

SEQ ID NO: 1214 - protein, 40 aa, Artificial Sequence
  Synthetic polypeptide
QLHHLKLSEDEETVYNVFFARSRSALQSYLKRHESRGNQS

SEQ ID NO: 1215 - protein, 40 aa, Artificial Sequence
  Synthetic polypeptide
LRVIGRGSYAKVLLVRLKKTDRIYAMKVVKKELVNDDEDI

SEQ ID NO: 1216 - protein, 40 aa, Artificial Sequence
  Synthetic polypeptide
NQSVEQMCNLLLKDQKPKKQGKYICEYCNRACAKPSVLLK

SEQ ID NO: 1217 - protein, 40 aa, Artificial Sequence
  Synthetic polypeptide
VDSGDSEVVDGLMLQHITLLMCSAYRNQLLNIFVRPSLVA

SEQ ID NO: 1218 - protein, 40 aa, Artificial Sequence
  Synthetic polypeptide
ELFFLDIHNIHVMRESLKKVKDIVYPNVEESHWLSSLEST

SEQ ID NO: 1219 - protein, 40 aa, Artificial Sequence
  Synthetic polypeptide
FDLDGDECLSHEEFLGVLKNRMHRGLWVPQHQSIQEYWKC

SEQ ID NO: 1220 - protein, 40 aa, Artificial Sequence
  Synthetic polypeptide
VLRGHEFLSHPFAVTLYGGEVYWTDWRTNTLAKANKWTGH

SEQ ID NO: 1221 - protein, 40 aa, Artificial Sequence
  Synthetic polypeptide
KAEDGATPSPSNETPKKKKKRFSFKKSFKLSGFSFKKNKK

SEQ ID NO: 1222 - protein, 40 aa, Artificial Sequence
  Synthetic polypeptide
SKAKAEKPPLSASSPQQRPPEPETGESAGTSRAATPLPSL

SEQ ID NO: 1223 - protein, 40 aa, Artificial Sequence
  Synthetic polypeptide
SAFNLLMHYPPPSGAGQHPQPQPPLHKANQPPHGVPQLSL

SEQ ID NO: 1224 - protein, 40 aa, Artificial Sequence
  Synthetic polypeptide
FHLVPNHIVVSAEGNISKKTECLGRALKFDKVGLVQYQST

SEQ ID NO: 1225 - protein, 40 aa, Artificial Sequence
  Synthetic polypeptide
TLTILRLSFCTNMEIPHFFCDPSEVLKLACSDTFINNIVM

SEQ ID NO: 1226 - protein, 40 aa, Artificial Sequence
  Synthetic polypeptide
DPLTKPMQYKVVVPKIGNILDLCTALSALSGIPADKMIVT

SEQ ID NO: 1227 - protein, 40 aa, Artificial Sequence; identical to SEQ ID NO: 1197
  Synthetic polypeptide

SEQ ID NO: 1228 - protein, 40 aa, Artificial Sequence
  Synthetic polypeptide
LSTRHVHLECRLQLWWCGGAPDSSIPDDHQGEKGQGGTEG

SEQ ID NO: 1229 - protein, 40 aa, Artificial Sequence
  Synthetic polypeptide
EARRRAHDQLLDLKSSLLKKADTLIGEIFNSVREELKFKH

SEQ ID NO: 1230 - protein, 40 aa, Artificial Sequence
  Synthetic polypeptide
IRTHTGEKPYECNICKVRFTRQDKLKVHMRKHTGEKPYLC

SEQ ID NO: 1231 - protein, 40 aa, Artificial Sequence
  Synthetic polypeptide
KGVQSLLMYKDGDSVLQRGGSLRAPALPSRSDRLQQRLPI

SEQ ID NO: 1232 - protein, 40 aa, Artificial Sequence
  Synthetic polypeptide
NRGEIKGSESATYVPVAPPTPAWQPEIKPEPAWHDQDETS

SEQ ID NO: 1233 - protein, 40 aa, Artificial Sequence
  Synthetic polypeptide
APLTMASPAMLGNVESGGPPPPTASQPASVNIPGSLPSST

SEQ ID NO: 1234 - protein, 40 aa, Artificial Sequence
  Synthetic polypeptide
QEFLHRYQELLDDNQAPFFLFTCAMRWLAVRLDLISIALI

SEQ ID NO: 1235 - protein, 40 aa, Artificial Sequence
  Synthetic polypeptide
TVDADTVTELAQVIVSRGGRFLEAPVSGNQQLSNDGMLVI

SEQ ID NO: 1236 - protein, 40 aa, Artificial Sequence
  Synthetic polypeptide
DPSCTVGFYAGDRKEFETLCSELTRVLSSSSATERYPMFT

SEQ ID NO: 1237 - protein, 40 aa, Artificial Sequence
  Synthetic polypeptide
KVQEQLKITNLRVQLLKRQSCPCQRNDLNEEPQHFTHYAI

SEQ ID NO: 1238 - protein, 40 aa, Artificial Sequence
  Synthetic polypeptide
NNTWEGHYYHYSDPVCKHPTFSIYARGRYSRGVLSSRVMG

SEQ ID NO: 1239 - protein, 40 aa, Artificial Sequence
  Synthetic polypeptide
RWRRRGQPMFLALDRRGGPRPGGRTRRYHLSAHFLPVLVS

SEQ ID NO: 1240 - protein, 40 aa, Artificial Sequence
  Synthetic polypeptide
EVPAAASQPTFLPWVPERGGGELDLVVRELQALEEELREA

SEQ ID NO: 1241 - protein, 40 aa, Artificial Sequence
  Synthetic polypeptide
GPALEEAAGPLVPGLVLGGFGKRKAPKVQPFLPRWLAEPN

SEQ ID NO: 1242 - protein, 40 aa, Artificial Sequence
  Synthetic polypeptide
AARGTWWNRPGGTSGSGEGVALGTTRKFQATGSRPAGEED

SEQ ID NO: 1243 - protein, 40 aa, Artificial Sequence
  Synthetic polypeptide
VAAGKAKKQVFYGEEERLKKPPRLQESCDSDHGGGRPAAA

SEQ ID NO: 1244 - protein, 40 aa, Artificial Sequence
  Synthetic polypeptide
DEHSSDPYHSGYEMPYAGGGGGPTYGPPQPWGHPDVHIMQ

SEQ ID NO: 1245 - protein, 43 aa, Artificial Sequence
  Synthetic polypeptide
LLDTKDQSHDLGLHVLSCRNNPLIIPVVHDLSQPFYHSQAVRV

(19) United States
(12) Patent Application Publication
(10) Pub. No.: US 2024/0218019 A1
(43) Pub. Date: Jul. 4, 2024

(54) METHODS AND COMPOSITIONS COMPRISING MHC CLASS I PEPTIDES

(71) Applicant: BOARD OF REGENTS, THE UNIVERSITY OF TEXAS SYSTEM, Austin, TX (US)

(72) Inventors: Eduardo VILAR SANCHEZ, Houston, TX (US); Kyle CHANG, Houston, TX (US); Wenhui WU, Houston, TX (US); Charles M. BOWEN, Houston, TX (US); Krishna M. SINHA, Houston, TX (US)

(73) Assignee: BOARD OF REGENTS, THE UNIVERSITY OF TEXAS SYSTEM, Austin, TX (US)

(21) Appl. No.: 18/554,180

(22) PCT Filed: Apr. 6, 2022
(86) PCT No.: PCT/US2022/023714

§ 371 Date: Oct. 5, 2023

Related U.S. Application Data
(60) Provisional application No. 63/171,137, filed on Apr. 6, 2021.

(51) Int. Cl. C07K 7/08; A61K 9/00; A61K 38/00; A61K 39/00; C07K 14/74; C12N 5/0783; C12N 5/0784; C12N 15/86

(52) U.S. Cl. CPC C07K 7/08; A61K 9/0073; A61K 39/4611; C07K 14/70539; C12N 5/0636; C12N 5/0639; C12N 15/86; A61K 38/00; C07K 2319/00; C12N 2710/10043

(57) ABSTRACT

The current disclosure provides methods and compositions for treating and vaccinating individuals against cancer. Accordingly, aspects of the disclosure relate to a peptide comprising at least 70% sequence identity to a peptide of one of SEQ ID NOS:1-776. In some embodiments, the peptide comprises at least 6 contiguous amino acids of a peptide of one of SEQ ID NOS:1-776. Further aspects relate to pharmaceutical compositions comprising the peptide, nucleic acids encoding the peptide, and expression vectors and host cells comprising the nucleic acids of the disclosure. Also provided is an in vitro dendritic cell comprising a peptide, nucleic acid, or expression vector of the disclosure.

This application claims benefit of priority of U.S. Provisional Application No. 63/171,137, filed Apr. 6, 2021, which is hereby incorporated by reference in its entirety.

### BACKGROUND OF THE INVENTION

### I. Field of the Invention

This invention relates to the field of treatment of cancer.

### II. Background

Lynch Syndrome (LS), the most common cause of hereditary colorectal cancer (CRC), represents 2-4% of total CRC and at least 1 million carriers in the United States (1). LS arises from heterozygous germline mutations in the DNA mismatch repair (MMR) genes, with MLH1 and MSH2 responsible for more than 70% of LS cases. LS patients have an increased lifetime risk for CRC development that reaches 60% in MLH1 and MSH2 carriers (2). Normal colorectal cells become MMR deficient (dMMR) upon acquisition of a second somatic hit in the alternative allele of the same MMR gene that harbors the germline mutation. This second hit manifests into base-to-base mismatches and insertion-deletion mutations (indels) in homopolymeric microsatellite sequences that are susceptible to indels. These mutations alter wild-type codon sequences and generate frameshift peptides (FSP) that are different from wild-type protein and thus become neoantigens (neoAg), which stimulate the adaptive immune system.

Tumor protein mutations (neoantigens) are processed into short peptides and presented on the cell surface complexed with major histocompatibility complex (MHC I/II). These peptides can bind to T cell receptors (TCRs) on cytotoxic CD8+ T cells, which promotes interferon γ (IFNγ) secretion in order to kill cancer cells. Thus, activation of CD8+ and CD4+ T cells (helper cell) recognizing neoantigens is important for adaptive immunity against tumors. Extensive system biology platforms and computational algorithms have used next-generation sequencing to rapidly screen the mutational landscape of human cancers, including melanoma and colon (3-6). Such studies have identified a variety of nonsynonymous mutations that may be recognized as foreign antigens to the host immune system providing promising avenues for more personalized and focused approaches to activate anti-tumor immunity (7)). For example, recent vaccination approaches employing mutated peptides to stimulate anti-tumor immunity have shown success in generating specific cytotoxic T lymphocyte (CTL) responses in human melanoma patients, and similar approaches in colorectal cancer have resulted in substantial tumor regressions (4).

Targeted therapies towards tumor-specific, frameshift neoantigens using the host immune system provide several advantages over previous and current immunotherapeutic strategies. For example, autoimmunity and dose-limiting toxicities have been reported in CRC patients receiving checkpoint inhibitors and adoptive T cell transfer against tumor-associated antigens (9-11). However, these immune-related adverse events become less problematic when targeting foreign neoantigens and cancer antigens through strategies such as immune vaccination (7). Furthermore, the significant deviation in sequence homology between frameshift neoantigens versus wild-type peptides has been hypothesized to elicit stronger immunogenic responses compared to viral and missense neoantigens, which further supports frameshift neoAg targeted therapies (12). Therefore, there is a need in the art for the development of compositions and methods for neoantigens identified from LS patients.

### SUMMARY OF THE INVENTION

The current disclosure fulfills a need in the art by providing methods and compositions for treating and vaccinating individuals against cancer through the use of newly identified immunogenic neoantigens. Accordingly, aspects of the disclosure relate to a peptide comprising at least 70% sequence identity to a peptide of one of SEQ ID NOS: 1-776. In some aspects, the peptide comprises at least 70% sequence identity to a peptide of one of SEQ ID NOS:10, 323, 221, 44, 27, 156, 37, 168, 20, 163, 29, 136, 24, 62, 138, 157, 160, 151, 158, 23, 39, or 57. Aspects of the disclosure relate to a peptide comprising at least or at most 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to a peptide of one of SEQ ID NOS:1-776. In some aspects, the peptide comprises at least 6 contiguous amino acids of a peptide of one of SEQ ID NOS: 1-776. Aspects of the disclosure relate to polypeptides comprising the peptides of the disclosure. Further aspects relate to pharmaceutical compositions comprising the peptide(s), polypeptide(s), virus, nucleic acids encoding the peptide or polypeptide, and expression vectors and host cells comprising the nucleic acids of the disclosure. In some aspects, the host cell may be a viral packaging cell. Aspects of the disclosure relate to a virus produced from a host cell of the disclosure. Also provided is an in vitro dendritic cell comprising a peptide, nucleic acid, or expression vector of the disclosure.

Further aspects relate to a method of making a cell comprising transferring a nucleic acid or expression vector of the disclosure into a cell, such as a host cell. The method may comprise or further comprise cultivating a cell having a nucleic acid or expression vector encoding any of the proteins discussed herein, including, but not limited to any of SEQ ID NOs:1-776. The method may comprise or further comprise isolating the expressed peptide or polypeptide. Other aspects of the disclosure relate to a method of producing cancer-specific immune effector cells comprising: (a) obtaining a starting population of immune effector cells; and/or (b) contacting the starting population of immune effector cells with a peptide or polypeptide of the disclosure, thereby generating peptide-specific immune effector cells.

The disclosure also describes peptide-specific engineered T cells produced according to the methods of the disclosure and pharmaceutical compositions comprising the engineered T cells. Further aspects relate to a method of treating or preventing cancer in a subject, the method comprising administering an effective amount of a peptide or polypeptide, pharmaceutical composition, nucleic acid, dendritic cell, or peptide-specific T cell of the disclosure. Yet further aspects relate to a method of cloning a peptide-specific T cell receptor (TCR), the method comprising (a) obtaining a starting population of immune effector cells; (b) contacting the starting population of immune effector cells with the peptide or polypeptide of the disclosure, thereby generating peptide-specific immune effector cells; (c) purifying immune effector cells specific to the peptide, and/or (d) isolating a TCR sequence from the purified immune effector cells. Also provide is a method for prognosing a patient or for detecting T cell responses in a patient, the method comprising: contacting a biological sample from the patient with a peptide or polypeptide of the disclosure.

Aspects of the disclosure also provide for a composition comprising at least one MHC polypeptide and a peptide of the disclosure and peptide-specific binding molecule that bind to a peptide of the disclosure or that bind to a peptide-MHC complex. Exemplary binding molecules include antibodies, TCR mimic antibodies, scFvs, nanobodies, camelids, aptamers, and DARPINs. Related methods provide for a method comprising contacting a composition comprising at least one MHC polypeptide and a peptide of the disclosure with a composition comprising T cells and detecting T cells with bound peptide and/or MHC polypeptide by detecting a detection tag. Further aspects relate to kits comprising a peptide, polypeptide, nucleic acid, expression vector, or composition of the disclosure.

In some aspects, the peptide is 13 amino acids in length or shorter. In some aspects, the peptide is 9 amino acids. The peptide may be at least, may be at most, or may consist of 7, 8, 9, 10, 11, 12, 13, 14, 15, 16, 17, 18, 19, or 20 amino acids (or any range derivable therein). The peptide may consist of 9 amino acids or the peptide may consist of 15 amino acids. The peptide may be further described as being immunogenic. The term immunogenic refers to the production of an immune response, such as a protective immune response. The peptide or polypeptide may be modified. The modification may comprise conjugation to a molecule. The molecule may be an antibody, a lipid, an adjuvant, or a detection moiety (tag). In some aspects, the peptide comprises 100% sequence identity to a peptide of one of SEQ ID NOS:1-776. Peptides of the disclosure also include those that have at least 90% sequence identity to a peptide of one of SEQ ID NOS: 1-776. The peptides of the disclosure may have 1, 2, or 3 substitutions relative to a peptide of one of SEQ ID NOS:1-776. In some aspects, the peptide has at least or at most 1, 2, 3, 4, or 5 substitutions relative to a peptide of one of SEQ ID NOS:1-776.

In some aspects, the nucleic acid of the disclosure is DNA. In some aspects, the nucleic acid of the disclosure is RNA. The RNA may be further defined as mRNA. The expression vector may comprise an adenoviral backbone. The expression vector may be a simian adenoviral vector, or a derivative thereof. In some aspects, the expression vector comprises a lentiviral expression vector.

The polypeptide may comprise at least 2 peptides of the disclosure. In some aspects, the polypeptide comprises, comprises at least, or comprises at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 peptides of the disclosure (or any derivable range therein). In some aspects, the polypeptide comprises four peptides of the disclosure. The polypeptide may comprise or further comprise a cell-penetrating peptide (CPP). The CPP may comprise the Z13 variant of ZEBRA CPP Z12. In some aspects, the polypeptide comprises or further comprises one or more TLR agonists. The TLR agonist may comprise a TLR2, TLR4, TLR2/4 agonist, or combinations thereof. The TLR agonist may comprise one or both of extra domain A (EDA) and Anaxa. In some aspects, the polypeptide comprises, from amino-proximal position to carboxy-proximal position: a cell penetrating peptide, one or more peptides of claims 1-12, and a TLR agonist. In some aspects, the polypeptide further comprises a TLR agonist amino-proximal to the cell penetrating peptide. Further aspects are described in Belnoue et al., JCI Insight. 2019; 4(11):e127305, which is herein incorporated by reference.

The pharmaceutical compositions of the disclosure may be formulated for parenteral administration, intravenous injection, intramuscular injection, inhalation, or subcutaneous injection. The peptides or polypeptides of the disclosure may be comprised in a liposome, lipid-containing nanoparticle, or in a lipid-based carrier. Pharmaceutical preparations may be formulated for injection or inhalation as a nasal spray. The compositions of the disclosure may be formulated as a vaccine. In some aspects, the composition may further comprise an adjuvant. In some aspects, the composition comprises at least 2 peptides of the disclosure. In some aspects, the composition comprises, comprises at least, or comprises at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 peptides of the disclosure (or any derivable range therein).

In some aspects, the polypeptide or composition comprises 4 different peptides, wherein each peptide is selected from a peptide of SEQ ID NO:10, 323, 221, 44, 27, 156, 37, 168, 20, 163, 29, 136, 24, 62, 138, 157, 160, 151, 158, 23, 39, and 57. In some aspects, the polypeptide or composition comprises, comprises at least, or comprises at most 2, 3, 4, 5, 6, 7, 8, 9, 10, 11, 12, 13, 14, 15, 16, 17, 18, 19, 20, 21, 22, 23, or 24 peptides (or any derivable range therein), wherein each peptide has an amino acid sequence of one of SEQ ID NO: 10, 323, 221, 44, 27, 156, 37, 168, 20, 163, 29, 136, 24, 62, 138, 157, 160, 151, 158, 23, 39, or 57

The dendritic cells of the disclosure may further be defined as being or as comprising mature dendritic cells. The cell may be a cell with an HLA-A type. The cell may also be a HLA-A, HLA-B, or HLA-C. In some aspects, the cell is an HLA-A3 or HLA-A11 type. In some aspects, the cell is an HLA-A01, HLA-A02, HLA-A24, HLA-B07, HLA-B08, HLA-B15, or HLA-B40. The methods may further comprise isolating the expressed peptide or polypeptide. The T cell may comprise a CD8+ T cell. The cell may be a T cell is a CD4+ T cell, a Th1, Th2, Th17, Th9, or Tfh T cell, a cytotoxic T cell, a memory T cell, a central memory T cell, or an effector memory T cell.

In methods of the disclosure, contacting may be further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface. The APCs may be, for example, dendritic cells.

The immune effector cells may be T cells, peripheral blood lymphocytes, natural killer (NK) cells, invariant NK cells, or NKT cells. The immune effector cells may be ones that have been differentiated from mesenchymal stem cell (MSC) or induced pluripotent stem (iPS) cells. The T cell aspects include T cells that are further defined as CD8⁺ T cells, CD4⁺ T cells, or γδT cells. The T cells may be defined as being cytotoxic T lymphocytes (CTLs).

The subject in the methods of the disclosure may be a human subject. The subject may also be a laboratory animal, a mouse, rat, pig, horse, rabbit, or guinea pig. Methods may further comprise administration of at least a second therapeutic agent. The second therapeutic agent may be an anti-cancer agent. Treating, as defined in the methods of the disclosure, may comprise one or more of reducing tumor size; increasing the overall survival rate; reducing the risk of recurrence of the cancer; reducing the risk of progression; and/or increasing the chance of progression-free survival, relapse-free survival, and/or recurrence-free survival.

The composition of the disclosure may comprise or further comprise a MHC polypeptide and a peptide of the disclosure and wherein the MHC polypeptide and/or peptide is conjugated to a detection tag. As such, suitable detection tags include, but are not limited to radioisotopes, fluorochromes, chemiluminescent compounds, dyes, and proteins, including enzymes. The tag may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent tags that produce signals include, but are not limited to bioluminescence and chemiluminescence. Examples of suitable fluorescent tags include, but are not limited to, fluorescein, rhodamine, tetramethylrhodamine, cosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.). Detection tags also include streptavidin or it's binding partner, biotin.

The MHC polypeptide and peptide may be operatively linked. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. Accordingly, aspects of the disclosure relate to wherein the MHC polypeptide and peptide are operatively linked through a peptide bond. The MHC polypeptide and peptide may also be operatively linked through van der Waals forces. The peptide-MHC may be operatively linked to form a pMHC complex. In some aspects, at least two pMHC complexes are operatively linked together. Other aspects include, include at least, or include at most 2, 3, 4, 5, 6, 7, 8, 9, or 10 pMHC complexes operatively linked to each other. In some aspects, at least two MHC polypeptides are linked to one peptide. In other aspects, the average ratio of MHC polypeptides to peptides is 1:1 to 4:1. In some aspects, the ratio or average ratio is at least, at most, or about 1, 2, 3, 4, 5, or 6 to about 1, 2, 3, 4, 5, or 6 (or any derivable range therein).

In some of the aspects of the disclosure, the peptide is complexed with MHC In some aspects, the MHC comprises HLA-A type. The MHC may be further defined as HLA-A3 or HLA-A11 type. The peptides may be loaded onto dendritic cells, lymphoblastoid cells, peripheral blood mononuclear cells (PBMCs), artificial antigen presentation cells (aAPC) or artificial antigen presenting surfaces. The artificial antigen presenting surface may comprise a MHC polypeptide conjugated or linked to a surface. Exemplary surfaces include a bead, microplate, glass slide, or cell culture plate.

Method of the disclosure may further comprise counting the number of T cells bound with peptide and/or MHC. The composition comprising T cells may be isolated from a patient having or suspected of having cancer. The cancer may comprise stage 0, I, II, III, or IV cancer. In some aspects, the cancer excludes stage 0, I, II, III, or IV cancer. The cancer may be colorectal cancer. The colorectal cancer may comprise comprises mismatch repair deficient colorectal cancer (MMR-d) and/or microsatellite instability (MSI) positive colorectal cancer. The subject being diagnosed or treated may be treated for stage I or stage II cancer. The subject may be one that has been determined to have mismatch repair deficient colorectal cancer (MMR-d) and/or microsatellite instability (MSI) positive colorectal cancer. The cancer may comprise a peptide-specific cancer, wherein the peptide of one of SEQ ID NOS:1-776 or a peptide of the disclosure. The subject may be a subject that has been diagnosed and/or determined to have a cancer. The subject or patient may also be one that has been characterized as having a peptide-specific cancer, such as a peptide of the disclosure or a peptide of one of SEQ ID NOS:1-776. The methods of the disclosure may comprise or further comprise comprises sorting the number of T cells bound with peptide and/or MHC. Methods of the disclosure may also comprise or further comprise sequencing one or more TCR genes from T cells bound with peptide and/or MHC. The methods may comprise or further comprise sequencing the TCR alpha and/or beta gene(s) from a TCR, such as a TCR that binds to a peptide of the disclosure. Methods may also comprise or further comprise grouping of lymphocyte interactions by paratope hotspots (GLIPH) analysis. This is further described in Glanville et al., Nature. 2017 Jul. 6; 547(7661): 94-98, which is herein incorporated by reference.

The compositions of the disclosure may be serum-free, mycoplasma-free, endotoxin-free, and/or sterile. The methods may further comprise culturing cells of the disclosure in media, incubating the cells at conditions that allow for the division of the cell, screening the cells, and/or freezing the cells. The methods may comprise or further comprise isolating the expressed peptide or polypeptide from a cell of the disclosure.

Methods of the disclosure may comprise or further comprise screening the dendritic cell for one or more cellular properties. The methods may comprise or further comprise contacting the cell with one or more cytokines or growth factors. The one or more cytokines or growth factors may comprise GM-CSF. The cellular property may comprise cell surface expression of one or more of CD86, HLA, and CD14. The dendritic cell may be derived from a CD34+ hematopoietic stem or progenitor cell.

The contacting in the methods of the disclosure may be further defined as co-culturing the starting population of immune effector cells with antigen presenting cells (APCs), wherein the APCs present the peptide on their surface. The APCs may be further defined as dendritic cells. The dendritic cell may be derived from a peripheral blood monocyte (PBMC). The dendritic cells may be isolated from PBMCs. The dendritic cells may also be cells in which the DCs are derived from are isolated by leukaphereses.

Peptide-MHC (pMHC) complexes of the disclosure may be made by contacting a peptide of the disclosure with a MHC complex. The peptide may be expressed in the cell and bind to endogenous MHC complex to form a pMHC. In some aspects, peptide exchange is used to make the pMHC complex. For example, cleavable peptides, such as photocleavable peptides may be designed that bind to and stabilize the MHC. Cleavage of the peptide (eg. by irradiation for photocleavable peptides) dissociates the peptide from the HLA complex and results in an empty HLA complex that disintegrates rapidly, unless UV exposure is performed in the presence of a “rescue peptide.” Thus, the peptides of the disclosure may be used as “rescue peptides” in the peptide exchange procedure. Also described herein are pMHC complexes comprising a peptide of the disclosure. The pMHC complex may be operatively linked to a solid support or may be attached to a detectable moiety, such as a fluorescent molecule, a radioisotope, or an antibody. Peptide-MHC multimeric complexes may include, may include at least or may include at most 1, 2, 3, 4, 5, or 6 peptide-MHC molecules operatively linked together. The linkage may be covalent, such as through a peptide bond, or non-covalent. The pMHC molecules may be bound to a biotin molecule. Such pMHC molecules may be multimerized through binding to a streptavidin molecule. pMHC multermers may be used to detect antigen-specific T cells or TCR molecules that are in a composition or in a tissue. The multimers may be used to detect peptide-specific T cells in situ or in a biopsy sample. Multimers may be bound to a solid support or deposited on a solid support, such as an array or slide. Cells may then be added to the slide, and detection of the binding between the pMHC multimer and cell may be conducted. Accordingly, the pMHC molecules and multimers of the disclosure may be used to detect and diagnose cancer in subjects or to determine immune responses in individuals with cancer.

In the methods of the disclosure, obtaining may comprise isolating the starting population of immune effector cells from peripheral blood mononuclear cells (PBMCs). The starting population of immune effector cells may be obtained from a subject. The subject may be one that has a cancer, such as a peptide-specific cancer. The subject may be one that has been determined to have a cancer that expresses a peptide of the disclosure. The methods of the disclosure may comprise or further comprise introducing the peptides or a nucleic acid encoding the peptide into the dendritic cells prior to the co-culturing. The introduction of the peptide may be done by transfecting or infecting dendritic cells with a nucleic acid encoding the peptide or by incubating the peptide with the dendritic cells. The peptide or nucleic acids encoding the peptide may be introduced by electroporation. Other methods of transfer of nucleic acids are known in the art, such as lipofection, calcium phosphate transfection, transfection with DEAE-dextran, microinjection, and virus-mediated transduction. The peptide or nucleic acids encoding the peptide may be introduced by adding the peptide or nucleic acid encoding the peptide to the dendritic cell culture media. The immune effector cells may be co-cultured with a second population of dendritic cells into which the peptide or the nucleic acid encoding the peptide has been introduced. In the methods of the disclosure, a population of CD4-positive or CD8-positive and peptide MHC tetramer-positive T cells may be purified from the immune effector cells following the co-culturing. The population of CD4-positive or CD8-positive and peptide MHC tetramer-positive T cells may be purified by fluorescence activated cell sorting (FACS). A clonal population of peptide-specific immune effector cells may be generated by limiting or serial dilution followed by expansion of individual clones by a rapid expansion protocol.

In the methods of the disclosure, purifying may comprise or further comprise generation of a clonal population of peptide-specific immune effector cells by limiting or serial dilution of sorted cells followed by expansion of individual clones by a rapid expansion protocol. Methods of the disclosure may comprise or further comprise cloning of a T cell receptor (TCR) from the clonal population of peptide-specific immune effector cells. The term isolating in the methods of the disclosure may be defined as or may comprise cloning of a T cell receptor (TCR) from the clonal population of peptide-specific immune effector cells. Cloning of the TCR may comprise cloning of a TCR alpha and a beta chain. The TCR may be cloned using a 5′-Rapid amplification of cDNA ends (RACE) method. The TCR alpha and beta chains may be cloned using a 5′-Rapid amplification of cDNA ends (RACE) method. The cloned TCR may be subcloned into an expression vector. The expression vector comprises may comprise a linker domain between the TCR alpha sequence and TCR beta sequence. The expression vector may be a retroviral or lentiviral vector. The vector may also be an expression vector described herein. The linker domain may comprise a sequence encoding one or more peptide cleavage sites. The one or more cleavage sites may be a Furin cleavage site and/or a P2A cleavage site. The TCR alpha sequence and TCR beta sequence may be linked by an IRES sequence.

A host cell of the disclosure may be transduced with an expression vector to generate an engineered cell that expresses the TCR alpha and/or beta chains. The host cell may be an immune cell. The immune cell may be a T cell and the engineered cell may be referred to as an engineered T cell. The T cell may be type of T cell described herein, such as a CD8+ T cell, CD4+ T cell, or γδ T cell. The starting population of immune effector cells may be obtained from a subject having a cancer or a peptide-specific cancer and the host cell is allogeneic or autologous to the subject. In some but not all aspects, obtaining a starting population of immune effector cells refers to retrieving them from the subject. The peptide-specific T cells may be autologous or allogeneic. In the methods of the disclosure, a population of CD4-positive or CD8-positive and peptide MHC tetramer-positive engineered T cells may be purified from the transduced host cells. A clonal population of peptide-specific engineered T cells may be generated by limiting or serial dilution followed by expansion of individual clones by a rapid expansion protocol. Purifying in the methods of the disclosure may be defined as purifying a population of CD4-positive or CD8-positive and peptide MHC tetramer-positive T cells from the immune effector cells following the co-culturing.

The peptide of the disclosure may be linked to a solid support. The peptide may be conjugated to the solid support or may be bound to an antibody that is conjugated to the solid support. The solid support may comprise a microplate, a bead, a glass surface, a slide, or a cell culture dish. The solid support may comprise a nanofluidic chip. In the methods of the disclosure, detecting T cell responses may comprise or further comprise detecting the binding of the peptide to the T cell or TCR. In the methods of the disclosure, detecting T cell responses may comprise or further comprise an ELISA, ELISPOT, or a tetramer assay.

Kits of the disclosure may comprise one or more peptides of the disclosure in a container. The peptide(s) may be comprised in a pharmaceutical preparation. The pharmaceutical preparation may be formulated for parenteral administration or inhalation. In some aspects, the peptide is comprised in a cell culture media.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more.” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments or aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a schematic depicting the in silico neoantigen prediction process.

FIG. 2 shows the in vitro validation pipeline.

FIG. 3 shows the analysis from whole exosome and RNA sequencing.

FIG. 4 shows a waterfall plot of the most recurrent neoantigens from MHC class I.

FIG. 5A-D shows the validation of neoantigen immunogenicity. FIG. 5A shows results from MHC tetramer staining. FIG. 5B shows the quantification of INFγ secreting cells. FIG. 5C shows cytotoxic gene expression. FIG. 5D shows the quantification of secreted cytokines from a multiplex ELISA based cytokine profile from CD8+ T cells after stimulation with neoAg-MHC.

FIG. 6. Schematic of the study

FIG. 7A-B. Mutational landscape in LS samples. A) Top panel shows the absolute count of each type of mutation per sample on the left y-axis, and the mutational burden (Mutations/MB) for each of the samples on the right x-axis. The middle grid panel shows the summary of mutations in selected genes. Each row is a gene and each column is a sample. Mutations are colored by type as shown in the legend on the right. The bar graph on the left of this summary of mutations represents the percentage of individuals with each specific gene mutated. The bottom panel displays molecular and pathological characteristics of each sample: MSI status (top), disease category (middle), and tissue pathology (bottom) as covariate bars. MSI-H=High microsatellite instability, MSI-L=Low microsatellite instability, MSS=Microsatellite stable, PRECA=Precancer, ADVPRECA=Advanced Precancer, CANCER=Cancer, AP=Adenoma polyp, ADVCA=Adenocarcinoma (Stage III &IV), SSA=Sesile serrated adenoma, HP=Hyperplastic polyp, IP=Inflammatory polyp, MB=megabase. B) A significant difference is observed in the mutational rate when samples are compared by MSI status (Mann Whitney test ****P-value >0.0001), by disease category (Mann Whitney test **P-value >0.01), as well as by tissue pathology (Mann Whitney test ***P-value >0.001).

FIG. 8A-E. A landscape of neoantigen produced from mutated proteins in LS patient cohort. A) There is a significant difference between the number of MHC I and MHC II NeoAg produced by the MSI samples compared to the MSI-L and MSS samples (Mann Whitney test ****P-value <0.0001). B) There is a significant difference between the number of MHC I and MHC II NeoAg produced by the cancers compared to the advanced pre-cancers and pre-cancers (Mann Whitney test ***P-value <0.001). C) There is a significant difference between the number of MHC I and MHC II NeoAg produced by the cancers compared to the other tissue pathologies (Mann Whitney test **P-value <0.01). D) The number of both MHC-I and II neoAgs detected per sample is significantly correlated with the mutational burden in each sample (Spearman P-value >0.0001). E) Waterfall plot for the most recurrent MHC class I neoantigens in the discovery set. The bar plot on top represents sample-wise neoAg rate (neoAg per Mb). The grid panel shows the top 50 most frequent MHC-I neoAg from the discovery set organized by the percentage of MSI-H sites for each gene (represented as the color scheme on the left). The in silico neoAg immunogenicity ranking is represented with the dark grey being the 1st percentile (highest predicted immunogenicity) and the light grey being the lowest-ranked. The bottom panel displays molecular and pathological characteristics of each sample: MSI status (top), discase category (middle), and tissue pathology (bottom) as covariate bars. MSI-H=High microsatellite instability, MSI-L=Low microsatellite instability, MSS=Microsatellite stable, PRECA=Precancer, ADVPRECA=Advanced Precancer, CANCER=Cancer, AP=Adenoma polyp, ADVCA=Adenocarcinoma (Stage III &IV), SSA=Sesile serrated adenoma, HP=Hyperplastic polyp, IP=Inflammatory polyp. The magenta asterisks indicate if that specific neoAg meets one TESLA presentation criteria (*), two TESLA presentation criteria (**), and three TESLA presentation criteria (***). The TESLA presentation criteria are: Binding affinity <34 nM, Tumor abundance >33TPM, Binding stability >1.4 h.

FIG. 9A-C. Shared neoAgs between the discovery set and the validation set. A) Venn diagram showing the number of neoAgs predicted from each set. B) Waterfall plot for the top 50 most recurrent MHC class I neoantigens from the validation set. The bar plot on top represents sample-wise neoAg rate (neoAg per Mb). The grid panel shows the top 50 most recurrent MHC I neoAg from the validation set organized by the percentage of MSI-H sites within that gene (represented as the color scheme on the left). The in silico neoAg immunogenicity ranking is represented as the grey scale, with dark grey being the 1st percentile (highest predicted immunogenicity) and light grey being the lowest-ranked. The neoAgs from the genes in light font were also present in the discovery set. The bottom panel displays molecular and pathological characteristics of each sample: MSI status (top), disease category (middle), and tissue pathology (bottom) as covariate bars. MSI-H=High microsatellite instability, MSI-L=Low microsatellite instability, MSS=Microsatellite stable, PRECA=Precancer, ADVPRECA=Advanced Precancer, CANCER=Cancer, AP=Adenoma polyp, ADVCA=Adenocarcinoma (Stage III &IV), SSA=Sesile serrated adenoma, HP=Hyperplastic polyp, IP=Inflammatory polyp. C) Each category of predicted neoags in the discovery set shows the percentage which are also present in the validation set.

FIG. 10A-D. In vitro validation of predicted neoAgs immunogenicity using ELISpot IFNy assay. A) Schematic of peptide pool, stimulation, and cell culture workflow for ELISpot assays. PBMCs were exposed with the individual peptides from the immunogenic pools in the presence of IL-7 for 3 d, followed by expansion of neoAg-specific T cells in the presence of IL-2. On day 13, expanded cells (10⁵ cells per well) were plated onto 96-well ELISpot plate coated with an IFNγ antibody and re-stimulated with the respective peptide for 24 h. IFNy secreting cells were analyzed as spot-forming units (SFUs), and the inventors chose ≥15 SFU produced by peptide stimulated cells over DMSO control cells as an indicator of the peptide immunogenicity. B) Quantification of IFNγ-secreting cells (SFU) obtained from ELISpot assay of 12 neoAg-stimulated PBMCs. ConcA and DMSO served as positive and negative controls. The bottom of the bar shows the representative image of the triplicate wells with IFNγ-secreting cells from three donors. C) Selection of 110 predicted MHC-I neoAgs from 3 different categories and percentage of immunogenicity in vitro validation. The number shown in parenthesis ( ) refers to the percentage of the tested neoAgs that showed immunogenicity in the ELISpot assay. “Most Immunogenic” refers to neoAgs selected from the top 100 prediction list of Most Immunogenic MHC-I neoAgs. “Most recurrent” refers to neoAgs selected from the top 100 prediction list of Most Recurrent MHC-I neoAgs. “Others” refers neoAgs that were predicted to have low immunogenicity and no recurrency. A total of 44 MHC-II neoAgs from 2 different categories were tested. The number shown in parenthesis ( ) refers to the percentage of the tested neoAgs that showed reactivity in the ELISpot assay. “Most Immunogenic” refers to neoAgs selected from the top 100 prediction list of Most Immunogenic MHC-II neoAgs. “Most recurrent” refers to neoAgs selected from the top 100 prediction list of Most Recurrent MHC-II neoAgs. D) pMHC-pentamer staining. Expanded Pan-T cells from the healthy human donor were isolated and stained with WDTC1 neoAg peptide/A*02:01 Pentameric complexes and PerCP-conjugated CD8 antibody followed by flow cytometry analysis. Pan T-cells from healthy human donor PBMC (HLA-A*02:01) were isolated using negative magnetic selection. Isolated Pan T cells were stimulated and expanded with Opto-antigen presenting beads conjugated with WDTC1 neoAg-peptide. Viable Pan-T cells were gated based on FSC and SSC scatter and SYTOX Blue dead cells staining after doublet-exclusion. Flow cytometric plots representing CD8 positive (x-axis) and PE-biotin-pMHC-pentamer positive (y-axis) cells; unstained, unstimulated, and WDTC1 Opto-antigen presenting beads stimulated cells (left to right). The percentage of live CD8+ cells and CD8/WDTC1 neoAg-loaded MHC pentamer positive cells are shown.

FIG. 11A-C. Differential gene expression analysis between cancers and precancers. A) Transcriptional expression profile of 78 differently expressed genes between cancer and precancers. Genes in light grey font are part of the immune response B) Pathway enrichment analysis showing activated and suppressed pathways in cancers compared to precancers. Pathways in light grey font are part of an immune response C) Immune cells showing a significant increase or decrease between cancers, advanced precancer and precancers after immune cell deconvolution. (Mann-Whitney test, *=p<0.05, **=p<0.01).

FIG. 12A-B. MSI status derived from MSI sensor results. A) The bar graph shows the number of microsatellite sites (left y-axis) with the unstable sites as the purple stacked bars and the stable sites as the grey stacked bars. The MSI score is shown as the dark grey circles (right y-axis). Samples with an MSI score equal or more than 10% are considered MSI-H. The bottom panel displays MSI status as covariate bars. B) MSI scores distribution by tissue categories. MSIscore >=10% means MSI-H, MSIscore <10% and >=3.5% means MSI-L, and MSIscore <3.5% means MSS.

FIG. 13. A landscape of second somatic hits in MMR gene. The grid panel shows the types of germline mutation (pastel color) present in one of the MMR genes for each sample, and the types of second somatic mutation as a black and white symbol, in the same samples. The bottom panel displays molecular and pathological characteristics of each sample: MSI status (top), disease category (middle), and pathology (bottom) as covariate bars. MSI-H=High microsatellite instability, MSI-L=Low microsatellite instability, MSS=Microsatellite stable, PRECA=Precancer, ADVPRECA=Advanced Precancer, CANCER=Cancer, AP=Adenoma polyp, ADVCA=Adenocarcinoma (Stage III & IV), SSA=Sessile serrated adenoma, HP=Hyperplastic polyp, IP=Inflammatory polyp.

FIG. 14. Neoantigen prediction pipeline. A schematic of the computational pipeline used to predict the MHC-I and MHC-II neoAgs from each sample. The final product of this pipeline is a list of ranked neoepitopes based on their immunogenicity scores.

FIG. 15. Most frequent HLA alleles in LS patient cohort. Percentage of samples covered by the top 80 most frequent HLA alleles.

FIG. 16. The number of neoantigens and their MHC binding affinity. The bar-graph shows the number of predicted MHC-I and -II neoAg with binding affinity <500 nM, 50-100 nM, 100-500 nM. The bottom panel displays molecular and pathological characteristics of each sample: MSI status (top), disease category (middle), and tissue pathology (bottom) as covariate bars. MSI-H=High microsatellite instability, MSI-L=Low microsatellite instability, MSS=Microsatellite stable, PRECA=Precancer, ADVPRECA=Advanced Precancer, CANCER=Cancer, AP=Adenoma polyp, ADVCA=Adenocarcinoma (Stage III &IV), SSA=Sesile serrated adenoma, HP=Hyperplastic polyp, IP=Inflammatory polyp.

FIG. 17. Waterfall plot for the top 50 most recurrent MHC class II neoantigens from the discovery set. The bar plot on top represents sample-wise neoAg rate (neoAg per Mb). The grid panel shows the top 50 most recurrent MHC II neoAg from the discovery set organized by the percentage of MSI-H sites within that gene. The in silico predicted neoAg immunogenicity ranking is represented as the scale, with dark grey being the 1st percentile (highest predicted immunogenicity) and light grey being the lowest-ranked. The bottom panel displays molecular and pathological characteristics of each sample: MSI status (top), disease category (middle), and tissue pathology (bottom) as covariate bars. MSI-H=High microsatellite instability, MSI-L=Low microsatellite instability, MSS=Microsatellite stable, PRECA=Precancer, ADVPRECA=Advanced Precancer, CANCER=Cancer, AP=Adenoma polyp, ADVCA=Adenocarcinoma (Stage III &IV), SSA=Sesile serrated adenoma, HP=Hyperplastic polyp, IP=Inflammatory polyp.

FIG. 18. The top 100 most immunogenic MHC-I predicted neoantigens from the discovery set meet the TESLA presentation and recognition criteria. TESLA determined five different peptide features or criteria that improve the performance of neoantigen prediction. These are binding affinity (Best.MTScore) <34 nM, tumor abundance (mt_allele_exp) >33 TPM. binding stability (Thalf(h)) >1.4 hours, agretopicity <0.1 and foreignness >10-16. The upset plot shows the number of neoantigens that pass different combinations of the five criteria. The aquamarine bar-graph on the left shows the total number of neoags that pass each criterion by itself.

FIG. 19A-B. In vitro validation of the pooled predicted neoAgs immunogenicity using ELISpot IFNγ assay. A) Schematic of peptide pool, stimulation, and cell culture workflow for ELISpot assays. All 154 peptides were grouped into 15 peptide pools to stimulate PBMCs from 3 healthy human donors as shown in the illustrations. IFNy secreting cells were analyzed as spot-forming units (SFUs), indicative of immunogenicity. B) Quantification of IFNγ-secreting cells (SFU) obtained from ELISpot assay. Pools 2, 3, 5, 6, 9, and 12 produced at least 15 or higher SFU/10⁵ cells from two different PBMCs. The bottom of the bar shows the representative image of the triplicate wells with IFNγ-secreting cells. ConcA and DMSO served as positive and negative controls.

FIG. 20. Other elispot reactive peptides. IFN-g ELISPOT SFU counts and well-images for neoAgs that showed lower ELISPOT reactivity agains human PBMCs from normal donors.

FIG. 21. MSI-H Sample coverage of most immunogenic MHC-I predicted neoags. The graph on the left shows the percentage of MSI-H samples that are covered by the top 100 most immunogenic MHC-I neoags, when these are ranked by recurrence, with the most recurrent ones considered first in the list. The graph on the right shows the percentage of MSI-H samples that are covered by the top 100 most immunogenic MHC-I neoags, when these are ranked only by the immunogenicity score, with the most immunogenic ones considered first in the list, even if they are not recurrent.

FIG. 22A-B. Validation of the predicted neoAg in LS Rhesus macaques. A) Immunogenicity of neoAg peptide pools and deconvoluted neoAg with ELISpot assay. PBMCs from LS Rhesus macaques (n=4) were stimulated with 10 peptide pools and 12 individual peptides from the pools, concavalin A (+ve control), and DMSO (−ve control) for 48 h. B) ELISpot images. All deconvoluted peptides except PLOD1 and CELSR2 were determined immunogenic in the stimulated rhesus PBMC. Spot forming Units for IFNγ secretion were analyzed and quantitated by ELISpot plate reader.

FIG. 23A-B. Unsupervised principal component analysis of all samples according to the gene expression results. A) Unsupervised PCA analysis of samples labeled based on their tissue category B) Unsupervised PCA analysis of samples labeled based on their MSI status.

FIG. 24A-C. Differential gene expression analysis between MSI-H and MSS samples. A) Transcriptional expression profile of 44 differently expressed genes between MSI-H and MSS samples. Genes in light grey font are part of an immune response. B) Pathway enrichment analysis showing activated and suppressed pathways in MSI-H compared to MSS samples. Pathways in light grey font are part of an immune response. C) Immune cells showing a significant increase or decrease between MSI-H, MSI-L and MSS samples after immune cell deconvolution. (Mann-Whitney test, *=p<0.05, **=p<0.01)

### DETAILED DESCRIPTION OF THE INVENTION

Lynch syndrome (LS) patients constitute a well-defined population that will likely benefit from cancer immune-interception strategies given they develop DNA mismatch repair-deficient tumors generating high loads of neoantigens (neoAgs). The examples of the application describe whole-exome sequencing and mRNAseq of colorectal cancers (CRC) and precancers of the LS patient cohort (N=46) to identify a landscape of somatic and genomic mutational variants and the prediction of highly immunogenic and recurrent neoantigens (neoAg) based on an immunogenicity score using in silico computational methods. This analysis revealed a positive correlation between microsatellite instability (MSI) and a high neoantigen load in precancerous and cancerous colorectal lesions. Furthermore, using ELISpot assays, the inventors tested 154 predicted neoAgs of high immunogenicity and recurrency, in vitro, from peripheral blood mononuclear cells (PBMC) of six healthy donors. These results showed that up to 50% of the predicted MHC-I frameshift neoAgs retained their immunogenicity, thus validating the neoAg prediction pipeline. Overall, the results from mutational and gene expression analyses of the catalogued neoAgs in this application help improve the understanding of LS-derived cancers, which will guide the future development of immunoprevention vaccine strategies.

### I. Immunotherapies Using Peptides of the Disclosure

A peptide as described herein (e.g., a peptide of one of SEQ ID NOS:1-776) may be used for immunotherapy of a cancer. For example, a peptide of one of SEQ ID NOS:1-776 may be contacted with or used to stimulate a population of T cells to induce proliferation of the T cells that recognize or bind said peptide. In other aspects, a peptide of the disclosure may be administered to a subject, such as a human patient, to enhance the immune response of the subject against a cancer.

A peptide of the disclosure may be included in an active immunotherapy (e.g., a cancer vaccine) or a passive immunotherapy (e.g., an adoptive immunotherapy). Active immunotherapies include immunizing a subject with a purified peptide antigen or an immunodominant peptide (native or modified); alternatively, antigen presenting cells pulsed with a peptide of the disclosure (or transfected with genes encoding an antigen comprising the peptide) may be administered to a subject. The peptide may be modified or contain one or more mutations such as, e.g., a substitution mutation. Passive immunotherapies include adoptive immunotherapies. Adoptive immunotherapies generally involve administering cells to a subject, wherein the cells (e.g., cytotoxic T cells) have been sensitized in vitro to a peptide of the disclosure (see, e.g., U.S. Pat. No. 7,910,109).

In some aspects, flow cytometry may be used in the adoptive immunotherapy for rapid isolation of human tumor antigen-specific T-cell clones by using, e.g., T-cell receptor (TCR) Vβ antibodies in combination with carboxyfluorescein succinimidyl ester (CFSE)-based proliferation assay. Sec, e.g., Lee et al., J. Immunol. Methods, 331:13-26, 2008, which is incorporated by reference for all purposes. In some aspects, tetramer-guided cell sorting may be used such as, e.g., the methods described in Pollack, et al., J Immunother Cancer. 2014; 2: 36, which is herein incorporated by reference for all purposes. Various culture protocols are also known for adoptive immunotherapy and may be used in aspects of the disclosure. In some aspects, cells may be cultured in conditions which do not require the use of antigen presenting cells (e.g., Hida et al., Cancer Immunol. Immunotherapy, 51:219-228, 2002, which is incorporated by reference). In other aspects, T cells may be expanded under culture conditions that utilize antigen presenting cells, such as dendritic cells (Nestle et al., 1998, incorporated by reference), and in some aspects artificial antigen presenting cells may be used for this purpose (Maus et al., 2002 incorporated by reference). Additional methods for adoptive immunotherapy are disclosed in Dudley et al. (2003), which is incorporated by reference, that may be used with aspects of the current disclosure. Various methods are known and may be used for cloning and expanding human antigen-specific T cells (see, e.g., Riddell et al., 1990, which is herein incorporated by reference).

In certain aspects, the following protocol may be used to generate T cells that selectively recognize peptides of the disclosure. Peptide-specific T-cell lines may be generated from normal donors or HLA-restricted normal donors and patients using methods previously reported (Hida et al., 2002). ENREE 32 Briefly, PBMCs (1×10⁵ cells/well) can be stimulated with about 10 μg/ml of each peptide in quadruplicate in a 96-well, U-bottom-microculture plate (Corning Incorporated, Lowell, MA) in about 200 μl of culture medium. The culture medium may consist of 50% AIM-V medium (Invitrogen), 50% RPMI1640 medium (Invitrogen), 10% human AB serum (Valley Biomedical, Winchester, VA), and 100 IU/ml of interleukin-2 (IL-2). Cells may be restimulated with the corresponding peptide about every 3 days. After 5 stimulations, T cells from each well may be washed and incubated with T2 cells in the presence or absence of the corresponding peptide. After about 18 hours, the production of interferon (IFN)-γ may be determined in the supernatants by ELISA. T cells that secret large amounts of IFN-γ may be further expanded by a rapid expansion protocol (Riddell et al., 1990; Yee et al., 2002b).

In some aspects, an immunotherapy may utilize a peptide of the disclosure that is associated with a cell penetrator, such as a liposome or a cell penetrating peptide (CPP). Antigen presenting cells (such as dendritic cells) pulsed with peptides may be used to enhance antitumour immunity (Celluzzi et al., 1996; Young et al., 1996). Liposomes and CPPs are described in further detail below. In some aspects, an immunotherapy may utilize a nucleic acid encoding a peptide of the disclosure, wherein the nucleic acid is delivered, e.g., in a viral vector or non-viral vector.

In some aspects, a peptide of the disclosure may be used in an immunotherapy to treat cancer in a mammalian subject, such as a human patient.

### II. Cell Penetrating Peptides

A peptide of the disclosure may also be associated with or covalently bound to a cell penetrating peptide (CPP). Cell penetrating peptides that may be covalently bound to a peptide of the disclosure include, e.g., HIV Tat, herpes virus VP22, the Drosophila Antennapedia homeobox gene product, signal sequences, fusion sequences, or protegrin I. Covalently binding a peptide to a CPP can prolong the presentation of a peptide by dendritic cells, thus enhancing antitumour immunity (Wang and Wang, 2002). In some aspects, a peptide of the disclosure (e.g., comprised within a peptide or polyepitope string) may be covalently bound (e.g., via a peptide bond) to a CPP to generate a fusion protein. In other aspects, a peptide or nucleic acid encoding a peptide, according to the current disclosure, may be encapsulated within or associated with a liposome, such as a multilamellar, vesicular, or multivesicular liposome.

As used herein, “association” means a physical association, a chemical association or both. For example, an association can involve a covalent bond, a hydrophobic interaction, encapsulation, surface adsorption, or the like.

As used herein, “cell penetrator” refers to a composition or compound which enhances the intracellular delivery of the peptide/polyepitope string to the antigen presenting cell. For example, the cell penetrator may be a lipid which, when associated with the peptide, enhances its capacity to cross the plasma membrane. Alternatively, the cell penetrator may be a peptide. Cell penetrating peptides (CPPs) are known in the art, and include, e.g., the Tat protein of HIV (Frankel and Pabo, 1988), the VP22 protein of HSV (Elliott and O'Hare, 1997) and fibroblast growth factor (Lin et al., 1995).

Cell-penetrating peptides (or “protein transduction domains”) have been identified from the third helix of the Drosophila Antennapedia homeobox gene (Antp), the HIV Tat, and the herpes virus VP22, all of which contain positively charged domains enriched for arginine and lysine residues (Schwarze et al., 2000; Schwarze et al., 1999). Also, hydrophobic peptides derived from signal sequences have been identified as cell-penetrating peptides. (Rojas et al., 1996; Rojas et al., 1998; Du et al., 1998). Coupling these peptides to marker proteins such as β-galactosidase has been shown to confer efficient internalization of the marker protein into cells, and chimeric, in-frame fusion proteins containing these peptides have been used to deliver proteins to a wide spectrum of cell types both in vitro and in vivo (Drin et al., 2002). Fusion of these cell penetrating peptides to a peptide of the disclosure may enhance cellular uptake of the polypeptides.

In some aspects, cellular uptake is facilitated by the attachment of a lipid, such as stearate or myristilate, to the polypeptide. Lipidation has been shown to enhance the passage of peptides into cells. The attachment of a lipid moiety is another way that the present invention increases polypeptide uptake by the cell.

A peptide of the disclosure may be included in a liposomal vaccine composition. For example, the liposomal composition may be or comprise a proteoliposomal composition. Methods for producing proteoliposomal compositions that may be used with the present invention are described, e.g., in Neclapu et al. (2007) and Popescu et al. (2007). In some aspects, proteoliposomal compositions may be used to treat a cancer.

By enhancing the uptake of a polypeptide of the disclosure, it may be possible to reduce the amount of protein or peptide required for treatment. This in turn can significantly reduce the cost of treatment and increase the supply of therapeutic agent. Lower dosages can also minimize the potential immunogencity of peptides and limit toxic side effects.

In some aspects, a peptide of the disclosure may be associated with a nanoparticle to form nanoparticle-polypeptide complex. In some aspects, the nanoparticle is a liposomes or other lipid-based nanoparticle such as a lipid-based vesicle (e.g., a DOTAP:cholesterol vesicle). In other aspects, the nanoparticle is an iron-oxide based superparamagnetic nanoparticles. Superparamagnetic nanoparticles ranging in diameter from about 10 to 100 nm are small enough to avoid sequestering by the spleen, but large enough to avoid clearance by the liver. Particles this size can penetrate very small capillaries and can be effectively distributed in body tissues. Superparamagnetic nanoparticles-polypeptide complexes can be used as MRI contrast agents to identify and follow those cells that take up the peptide. In some aspects, the nanoparticle is a semiconductor nanocrystal or a semiconductor quantum dot, both of which can be used in optical imaging. In further aspects, the nanoparticle can be a nanoshell, which comprises a gold layer over a core of silica. One advantage of nanoshells is that polypeptides can be conjugated to the gold layer using standard chemistry. In other aspects, the nanoparticle can be a fullerene or a nanotube (Gupta et al., 2005).

Peptides are rapidly removed from the circulation by the kidney and are sensitive to degradation by proteases in serum. By associating a peptide with a nanoparticle, the nanoparticle-polypeptide complexes of the present invention may protect against degradation and/or reduce clearance by the kidney. This may increase the serum half-life of polypeptides, thereby reducing the polypeptide dose need for effective therapy. Further, this may decrease the costs of treatment, and minimizes immunological problems and toxic reactions of therapy.

### III. Polyepitope Strings

In some aspects, a peptide is included or comprised in a polyepitope string. A polyepitope string is a peptide or polypeptide containing a plurality of antigenic epitopes from one or more antigens linked together. A polyepitope string may be used to induce an immune response in a subject, such as a human subject. Polyepitope strings have been previously used to target malaria and other pathogens (Baraldo et al., 2005; Moorthy et al., 2004; Baird et al., 2004). A polyepitope string may refer to a nucleic acid (e.g., a nucleic acid encoding a plurality of antigens including a peptide of the disclosure) or a peptide or polypeptide (e.g., containing a plurality of antigens including a peptide of the disclosure). A polyepitope string may be included in a cancer vaccine composition.

### IV. Applications of Antigenic Peptides

Various aspects are directed to development of and use of antigenic peptides that that are useful for treating and preventing certain cancers. In many aspects, antigenic peptides are produced by chemical synthesis or by molecular expression in a host cell. Peptides can be purified and utilized in a variety of applications including (but not limited to) assays to determine peptide immunogenicity, assays to determine recognition by T cells, peptide vaccines for treatment of cancer, development of modified TCRs of T cells, and development of antibodies.

Peptides can be synthesized chemically by a number of methods. One common method is to use solid-phase peptide synthesis (SPPS). Generally, SPPS is performed by repeating cycles of alternate N-terminal deprotection and coupling reactions, building peptides from the c-terminus to the n-terminus. The c-terminus of the first amino acid is coupled the resin, wherein then the amine is deprecated and then coupled with the free acid of the second amino acid. This cycle repeats until the peptide is synthesized.

Peptides can also be synthesized utilizing molecular tools and a host cell. Nucleic acid sequences corresponding with antigenic peptides can be synthesized. In some aspects, synthetic nucleic acids synthesized in in vitro synthesizers (e.g., phosphoramidite synthesizer), bacterial recombination system, or other suitable methods. Furthermore, synthesized nucleic acids can be purified and lyophilized, or kept stored in a biological system (e.g., bacteria, yeast). For use in a biological system, synthetic nucleic acid molecules can be inserted into a plasmid vector, or similar. A plasmid vector can also be an expression vector, wherein a suitable promoter and a suitable 3′-polyA tail is combined with the transcript sequence.

Aspects are also directed to expression vectors and expression systems that produce antigenic peptides or proteins. These expression systems can incorporate an expression vector to express transcripts and proteins in a suitable expression system. Typical expression systems include bacterial (e.g., E. coli), insect (e.g., SF9), yeast (e.g., S. cerevisiae), animal (e.g., CHO), or human (e.g., HEK 293) cell lines. RNA and/or protein molecules can be purified from these systems using standard biotechnology production procedures.

Assays to determine immunogenicity and/or TCR binding can be performed. One such as is the dextramer flow cytometry assay. Generally, custom-made HLA-matched MHC Class I dextramer:peptide (pMHC) complexes are developed or purchased (Immudex, Copenhagen, Denmark). T cells from peripheral blood mononuclear cells (PBMCs) or tumor-infiltrating lymphocytes (TILs) are incubated the pMHC complexes and stained, which are then run through a flow cytometer to determine if the peptide is capable of binding a TCR of a T cell.

The peptides of the disclosure can also be used to isolate and/or identify T-cell receptors that bind to the peptide. T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T-cell receptor differ from the membrane-bound immunoglobulin that serves as the B-cell receptor: a T-cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Cα domain, where the fold is unlike that of any other immunoglobulin-like domain. The half of the domain that is juxtaposed with the Cβ domain forms a β sheet similar to that found in other immunoglobulin-like domains, but the other half of the domain is formed of loosely packed strands and a short segment of α helix. The intramolecular disulfide bond, which in immunoglobulin-like domains normally joins two β strands, in a Cα domain joins a β strand to this segment of α helix.

There are also differences in the way in which the domains interact. The interface between the V and C domains of both T-cell receptor chains is more extensive than in antibodies, which may make the hinge joint between the domains less flexible. And the interaction between the Cα and Cβ domains is distinctive in being assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the B strand that anchors one end of the loop from one face of the domain to the other. A strand displacement also causes a change in the orientation of the Vβ CDR2 loop in two of the seven Vβ domains whose structures are known. As yet, the crystallographic structures of seven T-cell receptors have been solved to this level of resolution.

Aspects of the disclosure relate to engineered T cell receptors that bind a peptide of the disclosure, such as a peptide of one of SEQ ID NOS: 1-776. The term “engineered” refers to T cell receptors that have TCR variable regions grafted onto TCR constant regions to make a chimeric polypeptide that binds to peptides and antigens of the disclosure. In certain aspects, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some aspects, the TCR comprises non-TCR sequences. Accordingly, certain aspects relate to TCRs with sequences that are not from a TCR gene. In some aspects, the TCR is chimeric, in that it contains sequences normally found in a TCR gene, but contains sequences from at least two TCR genes that are not necessarily found together in nature.

### V. Antibodies

Aspects of the disclosure relate to antibodies that target the peptides of the disclosure, or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see. e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

### VI. Antibody Conjugates

Aspects of the disclosure relate to antibodies against a peptide of the disclosure, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium ¹⁸⁶, rhenium ¹⁸, ⁷⁵ selenium, ³⁵ sulphur, technicium^{99m} and/or yttrium ⁹⁰, ¹²⁵I is often being preferred for use in certain aspects, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other aspects, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

In another aspect of the disclosure, the antibody may be linked to semiconductor nanocrystals such as those described in U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999). In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AIS, AIP, AlSb, PbS, PbSe, Ge and Si and ternary and quaternary mixtures thereof. Methods for linking semiconductor nanocrystals to antibodies are described in U.S. Pat. Nos. 6,630,307 and 6,274,323.

In still further aspects, the present invention concerns immunodetection methods for binding, purifying, removing, quantifying and/or otherwise generally detecting biological components such as T cells or that selectively bind or recognize a peptide of the disclosure. In some aspects, a tetramer assay may be used with the present invention. Tetramer assays generally involve generating soluble peptide-MHC tetramers that may bind antigen specific T lymphocytes, and methods for tetramer assays are described, e.g., in Altman et al. (1996). Some immunodetection methods that may be used include, e.g., enzyme linked immunosorbent assay (ELISA), radioimmunoassay (RIA), immunoradiometric assay, fluoroimmunoassay, chemiluminescent assay, bioluminescent assay, tetramer assay, and Western blot. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

### VII. MHC Polypeptides

Aspects of the disclosure relate to compositions comprising MHC polypeptides. In some aspects, the MHC polypeptide comprises at least 2, 3, or 4 MHC polypeptides that may be expressed as separate polypeptides or as a fusion protein. Presentation of antigens to T cells is mediated by two distinct classes of molecules MHC class I (MHC-I) and MHC class II (MHC-II) (also identified as “pMHC” herein), which utilize distinct antigen processing pathways. Peptides derived from intracellular antigens are presented to CD8+ T cells by MHC class I molecules, which are expressed on virtually all cells, while extracellular antigen-derived peptides are presented to CD4+ T cells by MHC-II molecules. In certain aspects, a particular antigen is identified and presented in the antigen-MHC complex in the context of an appropriate MHC class I or II polypeptide. In certain aspects, the genetic makeup of a subject may be assessed to determine which MHC polypeptide is to be used for a particular patient and a particular set of peptides. In certain aspects, the MHC class 1 polypeptide comprises all or part of a HLA-A. HLA-B, HLA-C, HLA-E, HLA-F, HLA-G or CD-1 molecule. In aspects wherein the MHC polypeptide is a MHC class II polypeptide, the MHC class II polypeptide can comprise all or a part of a HLA-DR, HLA-DQ, or HLA-DP.

Non-classical MHC polypeptides are also contemplated for use in MHC complexes of the invention. Non-classical MHC polypeptides are non-polymorphic, conserved among species, and possess narrow, deep, hydrophobic ligand binding pockets. These binding pockets are capable of presenting glycolipids and phospholipids to Natural Killer T (NKT) cells or certain subsets of CD8+ T-cells such as Qa1, HLA-E-restricted CD8+ T-cells, or MAIT cells. NKT cells represent a unique lymphocyte population that co-express NK cell markers and a semi-invariant T cell receptor (TCR). They are implicated in the regulation of immune responses associated with a broad range of diseases.

### VIII. Host Cells

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed.” which refers to a process by which exogenous nucleic acid, such as a recombinant protein-encoding sequence, is transferred or introduced into the host cell. A transformed cell includes the primary subject cell and its progeny.

In certain aspects transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT. Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NSO, Panc-1, PC12, PC-3, Peripheral blood cells, Plasma cells, Primary Fibroblasts, RBL, Renca, RLE, SF21, SF9, SH-SY5Y, SK-MES-1, SK-N-SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### IX. Additional Agents

### A. Immunostimulators

In some aspects, the method further comprises administration of an additional agent. In some aspects, the additional agent is an immunostimulator. The term “immunostimulator” as used herein refers to a compound that can stimulate an immune response in a subject, and may include an adjuvant. In some aspects, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL®. (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalenc+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some aspects, the additional agent comprises an agonist for pattern recognition receptors (PRR), including, but not limited to Toll-Like Receptors (TLRs), specifically TLRs 2, 3, 4, 5, 7, 8, 9 and/or combinations thereof. In some aspects, additional agents comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some aspects, the additional agents also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some aspects, an additional agent may be a TLR-4 agonist, such as bacterial lipopolysaccharide (LPS), VSV-G, and/or HMGB-1. In some aspects, additional agents may comprise TLR-5 agonists, such as flagellin, or portions or derivatives thereof, including but not limited to those disclosed in U.S. Pat. Nos. 6,130,082, 6,585,980, and 7,192,725.

In some aspects, additional agents may be proinflammatory stimuli released from necrotic cells (e.g., urate crystals). In some aspects, additional agents may be activated components of the complement cascade (e.g., CD21, CD35, etc.). In some aspects, additional agents may be activated components of immune complexes. Additional agents also include complement receptor agonists, such as a molecule that binds to CD21 or CD35. In some aspects, the complement receptor agonist induces endogenous complement opsonization of the synthetic nanocarrier. In some aspects, immunostimulators are cytokines, which are small proteins or biological factors (in the range of 5 kD-20 kD) that are released by cells and have specific effects on cell-cell interaction, communication and behavior of other cells. In some aspects, the cytokine receptor agonist is a small molecule, antibody, fusion protein, or aptamer.

### B. Immunotherapies

In some aspects, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

### 1. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7. TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### 6. Checkpoint Inhibitors and Combination Treatment

In some aspects, the additional therapy comprises immune checkpoint inhibitors. Certain aspects are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC. Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some aspects, the inhibitor comprises the heavy and light chain CDRs or VRs of tremelimumab or ipilimumab. Accordingly, in one aspect, the inhibitor comprises the CDR1. CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### C. Oncolytic Virus

In some aspects, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

### D. Polysaccharides

In some aspects, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### E. Chemotherapies

In some aspects, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adrcocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluoride-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Other Agents

It is contemplated that other agents may be used in combination with certain aspects of the present aspects to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other aspects, cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present aspects to improve the treatment efficacy.

### X. Proteinaceous Compositions

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a peptide, protein, or polypeptide (wild-type or modified), such as a peptide or protein of the disclosure comprising a peptide of one of SEQ ID NOS: 1-1245 may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). It is specifically contemplated that any one or more peptides of one of SEQ ID NOS: 1-1245 may be excluded in in one or more aspects.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous in sequence to at least, or at most 3, 4, 5, 6, 7, 8, or 9 contiguous amino acids of a peptide of one of SEQ ID NOS:1-1245 or nucleic acids encoding a peptide of one of SEQ ID NOS: 1-1245. In certain aspects, the peptide or polypeptide is not naturally occurring and/or is in a combination of peptides or polypeptides.

In some aspects, the protein or polypeptide may comprise amino acids 1 to 2, 3, 4, 5, 6, 7, 8, 9, 10, 11, 12, 13, 14, 15, 16, 17, 18, 19, or 20 (or any derivable range therein) of a peptide of one of SEQ ID NOS:1-1245. In some aspects, the peptides of the disclosure comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) flanking the caboxy and/or flanking the amino end of a peptide comprising or consisting of 3, 4, 5, 6, 7, 8, 9, 10, 11, 12, 13, 14, 15, 16, 17, 18, 19, or 20 contiguous amino acids of a peptide of one of SEQ ID NOS: 1-1245.

In some aspects, the protein, polypeptide, or nucleic acid may comprise 1, 2, 3, 44, 5, 6, 7, 8, 9, 10, 11, 12, 13, 14, 15, 16, 17, 18, 19, or 20 (or any derivable range therein) contiguous amino acids of a peptide of one of SEQ ID NOS:1-1245.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, or 20 (or any derivable range therein) contiguous amino acids of a peptide of one of SEQ ID NOS:1-1245 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to a peptide of one of SEQ ID NOS:1-1245.

In some aspects there is a polypeptide (or a nucleic acid molecule encoding such a polypeptide) starting at position 1, 2, 3, 4, 5, 6, 7, 8, 9, 10, 11, 12, 13, 14, 15, 16, 17, 18, 19, or 20 of a peptide of one of SEQ ID NOS: 1-1245 and comprising at least, at most, or exactly 2, 3, 4, 5, 6, 7, 8, 9, 10, 11, 12, 13, 14, 15, 16, 17, 18, or 19 (or any derivable range therein) contiguous amino acids of a peptide of one of SEQ ID NOS: 1-1245.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

In some aspects, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, or 214 of the peptide or polypeptide of one of SEQ ID NOS:1-1245 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0+1); glutamate (+3.0+1); serine (+0.3); asparagine (+0.2); glutamine (+0.2); glycine (0); threonine (−0.4); proline (−0.5=1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of a polypeptide with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some aspects of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### XI. Nucleic Acids

In certain aspects, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene.” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the case of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another aspect, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. In a preferred aspect, the nucleic acid molecules are oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### XII. Polypeptide Expression

In some aspects, there are nucleic acid molecule encoding polypeptides or peptides of the disclosure (e.g antibodies, TCR genes, MHC molecules, and immunogenic peptides). These may be generated by methods known in the art, e.g., isolated from B cells of mice that have been immunized and isolated, phage display, expressed in any suitable recombinant expression system and allowed to assemble to form antibody molecules or by recombinant methods.

The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, antigenic peptides and polypeptides, TCR genes, MHC molecules, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### D. Host Cells

In another aspect, contemplated are the use of host cells into which a recombinant expression vector has been introduced. Polypeptides can be expressed in a variety of cell types. An expression construct encoding a polypeptide or peptide of the disclosure can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### XIII. Formulations and Culture of the Cells

In particular aspects, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V. X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain aspects, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine) . In specific aspects, one or more of these may be explicitly excluded.

In some aspects, the medium further comprises vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some aspects, the medium comprises one or more of the following: a B-27® supplement, xeno-free B-27® supplement, GS21™ supplement, or combinations thereof. In some aspects, the medium comprises or further comprises amino acids, monosaccharides, inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. . In specific aspects, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific aspects, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular aspects the cells are in a formulated cell suspension that is stable at room temperature for 1, 2, 3, or 4 hours or more from time of thawing.

In some aspects, the method further comprises priming the T cells. In some aspects, the T cells are primed with antigen presenting cells. In some aspects, the antigen presenting cells present tumor antigens or peptides, such as those disclosed herein.

In particular aspects, the cells of the disclosure comprise an exogenous TCR, which may be of a defined antigen specificity, such as defined antigen specificity to SEQ ID NO:1. In some aspects, the TCR can be selected based on absent or reduced alloreactivity to the intended recipient (examples include certain virus-specific TCRs, xeno-specific TCRs, or cancer-testis antigen-specific TCRs). In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some aspects, the choice of exogenous TCR may not necessarily be defined based on lack of alloreactivity. In some aspects, the endogenous TCR genes have been modified by genome editing so that they do not express a protein. Methods of gene editing such as methods using the CRISPR/Cas9 system are known in the art and described herein.

### XIV. Administration of Therapeutic Compositions

Methods of the disclosure relate to the treatment of subjects with cancer. In some aspects, the treatment may be directed to those that have or have been determined to have a cancer for a particular peptide of the disclosure, such as a peptide of one of SEQ ID NOS:1-776. In some aspects, the methods may be employed with respect to individuals who have tested positive for such cancer, who have one or more symptoms of a cancer, or who are deemed to be at risk for developing such a cancer.

The therapy provided herein may comprise administration of a combination of therapeutic agents, such as a first anti-cancer therapy and a second anti-cancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In select aspects, it is contemplated that a peptide of the disclosure may be comprised in a vaccine composition and administered to a subject to induce a therapeutic immune response in the subject towards a cancer. A vaccine composition for pharmaceutical use in a subject may comprise a peptide composition disclosed herein and a pharmaceutically acceptable carrier.

The phrases “pharmaceutical,” “pharmaceutically acceptable,” or “pharmacologically acceptable” refers to molecular entities and compositions that do not produce an adverse, allergic or other untoward reaction when administered to an animal, such as, for example, a human, as appropriate. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington: The Science and Practice of Pharmacy, 21st edition, Pharmaceutical Press, 2011, incorporated herein by reference). Except insofar as any conventional carrier is incompatible with the active ingredient, its use in the vaccine compositions of the present invention is contemplated.

As used herein, a “protective immune response” refers to a response by the immune system of a mammalian host to a cancer. A protective immune response may provide a therapeutic effect for the treatment of a cancer, e.g., decreasing tumor size, increasing survival, etc.

In some aspects, the vaccine composition may be administered by microstructured transdermal or ballistic particulate delivery. Microstructures as carriers for vaccine formulation are a desirable configuration for vaccine applications and are widely known in the art (Gerstel and Place 1976 (U.S. Pat. No. 3,964,482); Ganderton and McAinsh 1974 (U.S. Pat. No. 3,814,097); U.S. Pat. Nos. 5,797,898, 5,770,219 and 5,783,208, and U.S. Patent Application 2005/0065463). Such a vaccine composition formulated for ballistic particulate delivery may comprise an isolated peptide disclosed herein immobilized on a surface of a support substrate. In these aspects, a support substrate can include, but is not limited to, a microcapsule, a microparticle, a microsphere, a nanocapsule, a nanoparticle, a nanosphere, or a combination thereof.

In other aspects, a vaccine composition comprises an immobilized or encapsulated peptide or antibody as disclosed herein and a support substrate. In these aspects, a support substrate can include, but is not limited to, a lipid microsphere, a lipid nanoparticle, an ethosome, a liposome, a niosome, a phospholipid, a sphingosome, a surfactant, a transferosome, an emulsion, or a combination thereof. The formation and use of liposomes and other lipid nano- and microcarrier formulations is generally known to those of ordinary skill in the art, and the use of liposomes, microparticles, nanocapsules and the like have gained widespread use in delivery of therapeutics (e.g., U.S. Pat. No. 5,741,516, specifically incorporated herein in its entirety by reference). Numerous methods of liposome and liposome-like preparations as potential drug carriers, including encapsulation of peptides, have been reviewed (U.S. Pat. Nos. 5,567,434; 5,552,157; 5,565,213; 5,738,868 and 5,795,587, each of which is specifically incorporated in its entirety by reference).

In addition to the methods of delivery described herein, a number of alternative techniques are also contemplated for administering the disclosed vaccine compositions. By way of nonlimiting example, a vaccine composition may be administered by sonophoresis (i.e., ultrasound) which has been used and described in U.S. Pat. No. 5,656,016 for enhancing the rate and efficacy of drug permeation into and through the circulatory system; intraosseous injection (U.S. Pat. No. 5,779,708), or feedback-controlled delivery (U.S. Pat. No. 5,697,899), and each of the patents in this paragraph is specifically incorporated herein in its entirety by reference.

### XV. Detection and Vaccination Kits

A peptide or antibody of the disclosure may be included in a kit. The peptide or antibody in the kit may be detectably labeled or immobilized on a surface of a support substrate also comprised in the kit. The peptide(s) or antibody may, for example, be provided in the kit in a suitable form, such as sterile, lyophilized, or both.

The support substrate comprised in a kit of the invention may be selected based on the method to be performed. By way of nonlimiting example, a support substrate may be a multi-well plate or microplate, a membrane, a filter, a paper, an emulsion, a bead, a microbead, a microsphere, a nanobead, a nanosphere, a nanoparticle, an ethosome, a liposome, a niosome, a transferosome, a dipstick, a card, a celluloid strip, a glass slide, a microslide, a biosensor, a lateral flow apparatus, a microchip, a comb, a silica particle, a magnetic particle, or a self-assembling monolayer.

As appropriate to the method being performed, a kit may further comprise one or more apparatuses for delivery of a composition to a subject or for otherwise handling a composition of the invention. By way of nonlimiting example, a kit may include an apparatus that is a syringe, an eye dropper, a ballistic particle applicator (e.g., applicators disclosed in U.S. Pat. Nos. 5,797,898, 5,770,219 and 5,783,208, and U.S. Patent Application 2005/0065463), a scoopula, a microslide cover, a test strip holder or cover, and such like.

A detection reagent for labeling a component of the kit may optionally be comprised in a kit for performing a method of the present invention. In particular aspects, the labeling or detection reagent is selected from a group comprising reagents used commonly in the art and including, without limitation, radioactive elements, enzymes, molecules which absorb light in the UV range, and fluorophores such as fluorescein, rhodamine, auramine, Texas Red, AMCA blue and Lucifer Yellow. In other aspects, a kit is provided comprising one or more container means and a BST protein agent already labeled with a detection reagent selected from a group comprising a radioactive element, an enzyme, a molecule which absorbs light in the UV range, and a fluorophore.

When reagents and/or components comprising a kit are provided in a lyophilized form (lyophilisate) or as a dry powder, the lyophilisate or powder can be reconstituted by the addition of a suitable solvent. In particular aspects, the solvent may be a sterile, pharmaceutically acceptable buffer and/or other diluent. It is envisioned that such a solvent may also be provided as part of a kit.

When the components of a kit are provided in one and/or more liquid solutions, the liquid solution may be, by way of non-limiting example, a sterile, aqueous solution. The compositions may also be formulated into an administrative composition. In this case, the container means may itself be a syringe, pipette, topical applicator or the like, from which the formulation may be applied to an affected area of the body, injected into a subject, and/or applied to or mixed with the other components of the kit.

### XVI. Sequences

TABLE 1
Peptides

| | | SEQ | | | Binding | Tumor | Binding |
|---|---|---|---|---|---|---|---|
| Gene | | ID | Chromo- | HLA | Affinity | abundance | Stability |
| Name | Peptide | NO | some | Allele | (nM) | (TPM) | (hours) |
| USP9Y | YMMDDLELI | 1 | chrY | HLA- | 2 | 30.44 | 11.5 |
| | | | | A*02:01 | | | |
| TCF7L2 | SMMPPPPAL | 2 | chr10 | HLA- | 13.5 | 44.54 | 1.6 |
| | | | | A*02:01 | | | |
| WDR6 | FMNSTVFHV | 3 | chr3 | HLA- | 2 | 30.6 | 15.2 |
| | | | | A*02:01 | | | |
| TGFBR2 | SLVRLSSCVPV | 4 | chr3 | HLA- | 55.3 | 7.63 | 6.1 |
| | | | | A*02:01 | | | |
| MARCKS | FLQEVFQA | 5 | chr6 | HLA- | 51.5 | 6 | 2.7 |
| | | | | A*02:01 | | | |
| TAF1B | KLFEKKYSV | 6 | chr2 | HLA- | 3.8 | 7.27 | 14.2 |
| | | | | A*02:01 | | | |
| BCOR | SPAIPPRPL | 7 | chrX | HLA- | 5.9 | 3.7 | 4.7 |
| | | | | B*07:02 | | | |
| ZNF684 | KPIAGRHTL | 8 | chr1 | HLA- | 4.1 | 1.08 | 6 |
| | | | | B*07:02 | | | |
| SCNN1D | FLGHHSFSV | 9 | chr1 | HLA- | 2.3 | 0.68 | 14.8 |
| | | | | A*02:01 | | | |
| TTLL10 | FLIDDNFKV | 10 | chr1 | HLA- | 1.9 | 0.93 | 15 |
| | | | | A*02:01 | | | |
| NLRP3 | FLMDGFDEL | 11 | chr1 | HLA- | 1.9 | 1.32 | 5.6 |
| | | | | A*02:01 | | | |
| ABCA7 | FLWNSLLAV | 12 | chr19 | HLA- | 2.2 | 6.39 | 17.5 |
| | | | | A*02:01 | | | |
| CAMKK2 | RPRMRAASPL | 13 | chr12 | HLA- | 2 | 23.89 | 10.2 |
| | | | | B*07:02 | | | |
| DIDO1 | RPVRGRGSL | 14 | chr20 | HLA- | 2.4 | 46.37 | 7.1 |
| | | | | B*07:02 | | | |
| CHAT | RPLCSSLGPL | 15 | chr10 | HLA- | 6.4 | 0.54 | 5.2 |
| | | | | B*07:02 | | | |
| RNF128 | FLAPCNFYL | 16 | chrX | HLA- | 1.9 | 11.79 | 9.7 |
| | | | | A*02:01 | | | |
| RBM15 | TAPVASASPKL | 17 | chr1 | HLA- | 978.6 | 8.51 | 0.4 |
| | | | | B*07:02 | | | |

| | SEQ ID |
|---|---|
| Peptide Sequence | NO |
| YMMDDLELI | 1 |
| SMMPPPPAL | 2 |
| FMNSTVFHV | 3 |
| SLVRLSSCVPV | 4 |
| FLQEVFQA | 5 |
| KLFEKKYSV | 6 |
| SPAIPPRPL | 7 |
| KPIAGRHTL | 8 |
| FLGHHSFSV | 9 |
| FLIDDNFKV | 10 |
| FLMDGFDEL | 11 |
| FLWNSLLAV | 12 |
| RPRMRAASPL | 13 |
| RPVRGRGSL | 14 |
| RPLCSSLGPL | 15 |
| FLAPCNFYL | 16 |
| TAPVASASPKL | 17 |
| APREGAAATPL | 18 |
| KMMKILMIK | 19 |
| LSAPEKITLF | 20 |
| LSTEVQSLY | 21 |
| KLSSVVPSV | 22 |
| SLWSSMPHGV | 23 |
| TQLARFFPI | 24 |
| ALQSDVQPV | 25 |
| SLINIHHRK | 26 |
| RVPAHASTSL | 27 |
| IAQPSTSSL | 28 |
| MLLRLNLRK | 29 |
| APSWPDRPL | 30 |
| RLLPYPFHV | 31 |
| KMLTALPPA | 32 |
| KIKHGLSEK | 33 |
| YQMDFHPSPV | 34 |
| SPRPSACQL | 35 |
| QPHVPPSTL | 36 |
| RLYVPLYSSK | 37 |
| LSSPFREQM | 38 |
| IQKSWTATTY | 39 |
| FLDPDIGGV | 40 |
| FAMAQIQSL | 41 |
| RPRLPRHCL | 42 |
| YIMHLWPPI | 43 |
| FLATSGIDPV | 44 |
| TLDVELPPV | 45 |
| TLISMPYHV | 46 |
| SPMGRKQGGTL | 47 |
| RPKKSGDMTL | 48 |
| NMIQVLMSV | 49 |
| SLYGWYQLCV | 50 |
| STMRVAVTPK | 51 |
| RNLKNFLLMK | 52 |
| SLMEQIPHL | 53 |
| RTRGVCSVLK | 54 |
| IMHQYPNFK | 55 |
| KTVQAEPLI | 56 |
| KTYMEMHY | 57 |
| RSVLEEMGL | 58 |
| ALQEISFWL | 59 |
| RLFSFPAAK | 60 |
| KSLPSFLTM | 61 |
| KANRYFSPNF | 62 |
| SMASIMETI | 63 |
| TQGARSSAAF | 64 |
| FLRLDDLFKL | 65 |
| SLINLTWTA | 66 |
| MMIYFDMEV | 67 |
| LLKETKFITY | 68 |
| KSFHGLDFGF | 69 |
| RATFLLALW | 70 |
| IMMSWMPPL | 71 |
| KTHPCTMLL | 72 |
| APLFRASIL | 73 |
| WLWENHEKL | 74 |
| RRWECSHRL | 75 |
| SAFSSLLPL | 76 |
| FMQFSLFSV | 77 |
| WMIVTVLPV | 78 |
| FAFDSPHHY | 79 |
| GRLRVGLRLL | 80 |
| FLLTTLLGV | 81 |
| KRAARLVLR | 82 |
| VLSVRLPTRK | 83 |
| RMKHFIYFK | 84 |
| HRLRSLPRPL | 85 |
| FMDQEFLSFV | 86 |
| LLDDSNFKV | 87 |
| SPPVRSTVCAM | 88 |
| KADFRTLLK | 89 |
| FLAVDTQLL | 90 |
| RVYDPASPQR | 91 |
| RSLQAHKMAW | 92 |
| FLSPWPSPA | 93 |
| KRQKLICQM | 94 |
| SPPLHLCQPL | 95 |
| MTIYIFCLHY | 96 |
| RPACTCISM | 97 |
| LLLGCLCFI | 98 |
| RPENSQINSSL | 99 |
| SLMMIVLTI | 100 |
| NMMCQHTMI | 101 |
| YLTKWPKFFL | 102 |
| YSYPSSLSVF | 103 |
| TLWSRLVLA | 104 |
| LTSSQSSWW | 105 |
| ASLAHSDNF | 106 |
| AMAQVTHPL | 107 |
| KMNKILLPWK | 108 |
| QLRCWNTWAK | 109 |
| SLVTISRFV | 110 |
| YSDENMMDPY | 111 |
| FLALNQLPQV | 112 |
| KPRPLHAL | 113 |
| SLLSVGNLIGL | 114 |
| SQVWTAATLR | 115 |
| GMVPLIIPV | 116 |
| TPQDSRQVL | 117 |
| RAWRRFPLL | 118 |
| VGMRETTGL | 119 |
| TSSPRTMSW | 120 |
| SWMGGLHSFY | 121 |
| SQKNITPAI | 122 |
| KADQSESSL | 123 |
| LTHPAHQPL | 124 |
| RTLLVTCILY | 125 |
| RSAFPSRSL | 126 |
| VVHKKRGLF | 127 |
| LSWRGASFI | 128 |
| QSYNTVTRQW | 129 |
| WTGSCRQGW | 130 |
| KRAFIHTPR | 131 |
| LKLCSKVSF | 132 |
| KVDTHHLQV | 133 |
| SPSRSTTAPV | 134 |
| CRREYRVTM | 135 |
| WSWCGTSQTY | 136 |
| KPLWRKSPL | 137 |
| RLSCAPPPI | 138 |
| LSSWFSPTV | 139 |
| MSSIWGTMF | 140 |
| RTRSAWGDW | 141 |
| RTIMGWTLDF | 142 |
| ASRPGSFTF | 143 |
| KSLEGNLETF | 144 |
| LALPCRSVW | 145 |
| VLEFSSDRKK | 146 |
| KAFLPERKCF | 147 |
| TNTMGGVQGK | 148 |
| GSHNIKKAWY | 149 |
| AMAENILAA | 150 |
| YLGTPTWNC | 151 |
| RRPLRSWTPR | 152 |
| RAWRAGMPL | 153 |
| HSWRFCTHIR | 154 |
| MPCFTTALLL | 155 |
| QTIEERLTW | 156 |
| VMANVLTLNL | 157 |
| VLEDTLLKI | 158 |
| KLYEAVPQL | 159 |
| AGIGWGASY | 160 |
| RMASTSCAA | 161 |
| TPRKLVGRAV | 162 |
| WLPKMPPFV | 163 |
| SQNWGSLPL | 164 |
| GLLHAVQEKL | 165 |
| ATLVTPPTRY | 166 |
| IAFSQLIGM | 167 |
| LSNVAPPAF | 168 |
| KVPFFSALK | 169 |
| RFCPASCSGCY | 170 |
| RTHPYSPKK | 171 |
| VSNIAQAPLY | 172 |
| YVAIRPLPY | 173 |
| MYFFWPCSL | 174 |
| ILFFFSSK | 175 |
| HTCKVCVSF | 176 |
| LAYWEKREAW | 177 |
| HNVQGFHPY | 178 |
| SMAASPSPK | 179 |
| RAFSTFPSF | 180 |
| KSVRGLELL | 181 |
| QSSLSEKKF | 182 |
| RSLMSVASAY | 183 |
| GTNFWGVPRK | 184 |
| LSYNLGAGEAL | 185 |
| ITSPALLL | 186 |
| SSNSCASAF | 187 |
| GSYPSGSPCVW | 188 |
| HSASNGTPL | 189 |
| KLVGRAVRRK | 190 |
| ILKEAPRRK | 191 |
| STSFLDTRF | 192 |
| KHTEKKSLSF | 193 |
| FTHFHGEIW | 194 |
| AVLGTMVMK | 195 |
| QAGTPVMMF | 196 |
| MTLFSLVPL | 197 |
| SSLGTSDPRW | 198 |
| HSLVQMEPL | 199 |
| LTFCTNATI | 200 |
| YLFAKAYLV | 201 |
| VPACSHVPM | 202 |
| VPTWSARLL | 203 |
| LLNPVTMNK | 204 |
| SELLNPVTMNK | 205 |
| LLNPVTMNKA | 206 |
| ELLNPVTMNK | 207 |
| HPARHLCRL | 208 |
| APSLTPMHSL | 209 |
| SPMITRIL | 210 |
| TPMHSLLISPM | 211 |
| TPMHSLLIS | 212 |
| RLYKYDHNFV | 213 |
| RQMLPTLSTL | 214 |
| RLDKGNFAGA | 215 |
| MLLELSPAQL | 216 |
| HPPPPCLLLL | 217 |
| RWMVLRNSWRAVARM | 218 |
| IDNIKRNHNLALGRQ | 219 |
| PKMQVTITLTSPIIR | 220 |
| FQVHFLKSGGLPLVL | 221 |
| KTGLQLLRNHIEELK | 222 |
| QKKLMLLRLNLRKMC | 223 |
| LINIHHRKNPLLPMR | 224 |
| WILHLLGLRPPSLLS | 225 |
| LKETKFITYRSKKLI | 226 |
| EDIEFHFSLGWTMLV | 227 |
| GKNGFLQSRSSSLFS | 228 |
| PALLLAEATHKASAL | 229 |
| SLDNVLRTMLRRFAR | 230 |
| YLRFIKSLAERTMSV | 231 |
| KKDFGKMTANSVSVA | 232 |
| RHVIKVLLGRKVNWH | 233 |
| QLARFFPITPPVWHI | 234 |
| NAILLFLRTRGVCSV | 235 |
| FEEIIKNDGALLKKK | 236 |
| LRLLSLYRPPLAPLL | 237 |
| TKFITYRSKKLIQES | 238 |
| LEVMLLNMGYRITGL | 239 |
| SINVLCVRASLIEKL | 240 |
| DDVLRNLKNFLLMKR | 241 |
| LGKLEMVKAVQLRVA | 242 |
| TARISVNSNNVQSLL | 243 |
| SPMALLLAARQRAQK | 244 |
| GKVIMPLGSKLTGVI | 245 |
| KKVRVIYTQLSKTVV | 246 |
| AFFMNLTREPSRVLK | 247 |
| PSKRSLLSVGNLIGL | 248 |
| PLPTALRQLRGRPAD | 249 |
| TVEMRRWWTLVMEWK | 250 |
| NKKMLTALPPAMTAM | 251 |
| KKKRQINRRKLQRKK | 252 |
| LKMVWRINPAHRKLQ | 253 |
| PHRLRSLPRPLHLRL | 254 |
| NONLYLVGASKIRML | 255 |
| FHPYRRYPPPAAAAL | 256 |
| ESNLLQSPSSILSTL | 257 |
| LWGVRMTSLSASTSL | 258 |
| LSSLVKKILAMTLTL | 259 |
| KKTVLSLVTISRFVL | 260 |
| NSVIVGNTHGQLAEI | 261 |
| QLPVYKLLPSQNRLQ | 262 |
| IRKGFQLRKTARGRG | 263 |
| GVFISKVLPRGLAAR | 264 |
| QRRLIKSMESVMVKY | 265 |
| KKNILNSLPSSMEIA | 266 |
| NRVFKLAPNLTELRA | 267 |
| KLICQMTRTNRLFGM | 268 |
| NSKLRYKKRGVIAWR | 269 |
| SFASMGMLEARIRIL | 270 |
| DARLRASTALLLPIL | 271 |
| ARLCLIVSRTLLLVQ | 272 |
| ALSVLTASLSYMVGM | 273 |
| AWFIRESMTIYIFCL | 274 |
| ERLLFFAVPPQILAS | 275 |
| MFFMVFLIIWQNTMF | 276 |
| VFFAYLVAHSFLSVF | 277 |
| GTGASMASIMETIGL | 278 |
| LLPYPFHVLALEVTF | 279 |
| PLRICVTLWSRLVLA | 280 |
| ERVQTVAASTMRVAV | 281 |
| YQYTVFLRSDSYMGL | 282 |
| QRYRSVLRGWWILLT | 283 |
| LYGWYQLCVSSMKLL | 284 |
| TGATCGKRAARLVLR | 285 |
| LSDIYLNNVIMRFMQ | 286 |
| RLFLLQDSGRILQLL | 287 |
| APQVTRLRSLNHLLI | 288 |
| GIFLVIETHGMAVSW | 289 |
| ANRYFSPNFKVKLYF | 290 |
| WRLFLIIQTTGYQSI | 291 |
| GSQSIMMSWMPPLAP | 292 |
| GNLLSFSRRGMKSSV | 293 |
| HLTLARMKHFIYFKH | 294 |
| YTIFYRTIIGNETAV | 295 |
| WKVKLPSSMSVALPL | 296 |
| FQELILNQASMAPPR | 297 |
| KERLFRNFGGLLGPL | 298 |
| YSKVRALGGVNAARR | 299 |
| MMIVLTIQNAAFLSN | 300 |
| LIWIVFISSGHVASA | 301 |
| EPFIQKDVELRIMPP | 302 |
| GRLQIMSLENLSIEK | 303 |
| WPITELKIQMRGILG | 304 |
| RELLKTLNMIQVLMS | 305 |
| SAFSSLLPLRNLSQL | 306 |
| ILGSFFMATSSHRFL | 307 |
| RSRLMRQSRRSTQGV | 308 |
| PSWPMAVPLAASRAS | 309 |
| KLQAVLEDTLLKILL | 310 |
| KSLVRLSSCVPVALM | 311 |
| ACRLRWARPEPAAQA | 312 |
| IKICILWNQIMHASW | 313 |
| HVPLSISGSPALELL | 314 |
| HKKLILEKSPINVKK | 315 |
| EKDLMQLAQATAVAA | 316 |
| FTIYSISSLKTLFRK | 317 |
| LRGGVIQSTRRRRRA | 318 |
| PCASLLSTLSQPPPQ | 319 |
| EDMQEVVVHKKRGLF | 320 |
| RFPLLMMWRTPMTTR | 321 |
| PVLFKADQSESSLSS | 322 |
| RIPAVLRTEGEPLHT | 323 |
| FGILNVLEFSSDRKK | 324 |
| ILVALSWMGGLHSFY | 325 |
| LGQIMASAVEASQPP | 326 |
| QRWLTSTTSRSSALM | 327 |
| LLHWRIGGGTPLSIS | 328 |
| EIQLTMNDSKHKLES | 329 |
| LTCALHNDGIYIMSR | 330 |
| CILVALSWRGASFIL | 331 |
| GVRILKLCSKVSFRV | 332 |
| LRAVVVDDYRRRKKR | 333 |
| ALGLRILPPPLTSPS | 334 |
| KKSVLKAIEQADLLQ | 335 |
| KEALFLQEVFQAERL | 336 |
| WTPRKLVGRAVRRKG | 337 |
| ADRAFMAAQKCHKKT | 338 |
| FGYATSISMAQASDG | 339 |
| KYRCFSYLPISPTFV | 340 |
| LRGELSYNLGAGEAL | 341 |
| ARHRLASFKTVIKKR | 342 |
| LRELRLDNSVAIHYI | 343 |
| AAPEIILGNPVSLTS | 344 |
| RLSLVQSSSWPTVLH | 345 |
| SRQPSPLLLLPPLPA | 346 |
| KRLMSLSPGRPPLLL | 347 |
| KSLLFPSAPASVMNA | 348 |
| QGKLQQHHVLRVSRR | 349 |
| AEIRAQDAPLSLLQT | 350 |
| HGSHNIKKAWYLIAM | 351 |
| RCCLRTSCGAARPRR | 352 |
| LCNRLLKSFSKWSLV | 353 |
| PVTPLRVQSVLLLGV | 354 |
| IAGYRESAAFLLRSA | 355 |
| SGTARLARTAIAAST | 356 |
| NOPVEFNHAINYLIR | 357 |
| RRPLRSWTPRTRGAH | 358 |
| NEIQKLQKTLKKKPR | 359 |
| PRLVKMISSISLEIW | 360 |
| SSIFIGGSFILKKKA | 361 |
| PALLLPATTCKVPRL | 362 |
| KKRKKFMKDAKKRGR | 363 |
| REYMLNLFKALKRIH | 364 |
| APQGFRATLVPPALE | 365 |
| TTVGLLRMAATRTSL | 366 |
| DTWAALEGLSSPFRE | 367 |
| KQGWLHKKGGGLLHA | 368 |
| LTETVYSTTQQIHSS | 369 |
| NPAFDVTPTTSSLVA | 370 |
| ETNMGIIAGVAFGIA | 371 |
| SSFFCRCRREYRVTM | 372 |
| PGCFWPCLWNSLLRF | 373 |
| FPILFFFSSKGVRAT | 374 |
| IAYYIEGIENSVFFF | 375 |
| YDCYVAIRPLPYATR | 376 |
| DRINANTITSPALLL | 377 |
| HAFIVPIRSLQDHTH | 378 |
| PKKKRSAFPSRSLSS | 379 |
| ILLGPLLPNVVFYIL | 380 |
| PPGVVLNNISSYASV | 381 |
| WEAAMMNGKVPFFSA | 382 |
| SLQSAVSNIAQAPLY | 383 |
| RPQVRLAGAQAIFEA | 384 |
| PRFSQIHSILSKMVQ | 385 |
| KETLRLNPPVPGGFR | 386 |
| SRRSLMSVASAYSAK | 387 |
| REAGRFAVLGTMVMK | 388 |
| HSTPFQAGTPVMMFL | 389 |
| RLMPKFLNSTNSWWT | 390 |
| LATAAAAAAAAAFGD | 391 |
| KRLSKVETLRAAIDY | 392 |
| LAQEQKKKKSWRLLL | 393 |
| ITRQQWKKALRSMPK | 394 |
| SRPLAHSVASTLAPA | 395 |
| IGSISRQSSLSEKKF | 396 |
| GLCILKEAPRRKRPA | 397 |
| RKMILSQLHHSMTRF | 398 |
| ILQHFLLHATPQTQL | 399 |
| QAHLPSAPALPPPTH | 400 |
| GHSLVQMEPLTTARP | 401 |
| GVHSTIKVIKAKKKH | 402 |
| QGGLLMGYSPAGGRH | 403 |
| LLRMTLFSLVPLEPI | 404 |
| KSAFATYKVK | 405 |
| NLHLKTTSL | 406 |
| FLRQRATSTI | 407 |
| LIKLKLNRL | 408 |
| LMLLRLNL | 409 |
| HFINVMFVR | 410 |
| SALVRLFPV | 411 |
| FPKKKCTNL | 412 |
| MSVKKVMTY | 413 |
| FATYKAKMPL | 414 |
| MTLSKMIKK | 415 |
| NPRRKTWKM | 416 |
| SQKKRRYSI | 417 |
| KTTNHSSQM | 418 |
| AAICTTPAL | 419 |
| TATETKTPY | 420 |
| RIKKKLMEL | 421 |
| HSYHLLQAY | 422 |
| LSSVSFFLY | 423 |
| KRATFLLAL | 424 |
| YYYGGNCGLFY | 425 |
| LTMKEAVPK | 426 |
| AEISSQVPHW | 427 |
| AEKCLILVW | 428 |
| TELGILTSF | 429 |
| SLRRKYLRV | 430 |
| SQFLTEGIMK | 431 |
| LPLLRHHLPL | 432 |
| FSDSEGEGL | 433 |
| ITIGVRPIR | 434 |
| YADQWTVL | 435 |
| HTMWHCALEK | 436 |
| SAKKRASV | 437 |
| VVQKVAWFYK | 438 |
| HPSGGPIPL | 439 |
| KTVDHKVERK | 440 |
| TYTICVTMPY | 441 |
| TLRKKKQTV | 442 |
| EEKMYYLFVY | 443 |
| AENEKKILY | 444 |
| HSPTPTSAL | 445 |
| AEQEQEVVAW | 446 |
| WPIQRCACSV | 447 |
| HQKRRKKNL | 448 |
| TLKTLFHLR | 449 |
| KSFTKNHSSK | 450 |
| MIKNRISPL | 451 |
| NIKKKPGTSL | 452 |
| AESLRENFSW | 453 |
| TSVGLAWRW | 454 |
| CLSLRKKAL | 455 |
| VIIKKNPALPK | 456 |
| GALPRIHNM | 457 |
| AESGPFRPGW | 458 |
| TSLARPPPL | 459 |
| TPESPPLQLW | 460 |
| KALADPSAF | 461 |
| KRKSILLHL | 462 |
| YSTEKRKKY | 463 |
| RRRPRLPTL | 464 |
| AMQDFFSYY | 465 |
| TEVTWWALRK | 466 |
| CTACHTALGR | 467 |
| IEFRIKFLF | 468 |
| ITMQQIAVL | 469 |
| CLRPHRVQL | 470 |
| KTHPIRTSL | 471 |
| HAVGCPVQM | 472 |
| TALVPPPAL | 473 |
| WIIPFWFPF | 474 |
| FTVITYFLW | 475 |
| LFLVREVQR | 476 |
| SSPPFHYPF | 477 |
| RLHCQHSSL | 478 |
| NSKKTNATF | 479 |
| TKKRKMQSL | 480 |
| DLVPSCHPR | 481 |
| KMQFRLLVLL | 482 |
| TLKRRTLAM | 483 |
| TPALREYTM | 484 |
| ATGLNIWKLK | 485 |
| TTTCPSRPL | 486 |
| FGMMSMASR | 487 |
| FALCGFWQI | 488 |
| QVYSVPHFFF | 489 |
| SAASYLWPSR | 490 |
| QLKKKHLKA | 491 |
| GAKKHFGSF | 492 |
| FPRSQHQSPL | 493 |
| STAAPPAPR | 494 |
| YLFEGAQTV | 495 |
| TVLRVDQIMAK | 496 |
| ELSICIRIPR | 497 |
| KTYTCAITTVK | 498 |
| WLEKKNCYSL | 499 |
| RSVLWERVV | 500 |
| HLLQEYLPL | 501 |
| CPSPGPPSL | 502 |
| MSVCFFFFCY | 503 |
| VMSDTTYKIY | 504 |
| QAHPQVPAL | 505 |
| FPITPPVWHIL | 506 |
| VVHKKRGL | 507 |
| SRYPNICWF | 508 |
| MTTISRATW | 509 |
| SSWATCWPR | 510 |
| RVRAHPGLPR | 511 |
| STAHIPPLHLR | 512 |
| RLWNKTRCR | 513 |
| SSFLVSSSI | 514 |
| TFMPPPGHPPR | 515 |
| KTNATFSLV | 516 |
| AQTRDRWRK | 517 |
| STEVEETQEK | 518 |
| TFKKKTYTC | 519 |
| LPPPGCGSW | 520 |
| HARGSSLITM | 521 |
| MNKDLLRVL | 522 |
| VAARWHGTL | 523 |
| VAISFKTVF | 524 |
| QTHWRLYPK | 525 |
| LTMAPSCLR | 526 |
| MPQACDGLTW | 527 |
| SAWTGSVSV | 528 |
| YPAASAPCW | 529 |
| LPSSTLWTF | 530 |
| LPCNVTFLM | 531 |
| YPTSVHYQTPW | 532 |
| NSLPPAALR | 533 |
| VAHDPPQSL | 534 |
| SASGSPWPM | 535 |
| LLFPASGEM | 536 |
| APAPPTRCVW | 537 |
| SPQAPLRLW | 538 |
| SSHPRPLPA | 539 |
| SSARGWAPCK | 540 |
| IAKKMTSTL | 541 |
| RAMPSSGAA | 542 |
| RTFCLTARR | 543 |
| WACPHPRSL | 544 |
| FGPSLVRGLW | 545 |
| SASSLAAAL | 546 |
| SMRSTRWAR | 547 |
| ATLSAAFARR | 548 |
| SPFPPSSLHW | 549 |
| TAAACPTPV | 550 |
| EAAPPSTSM | 551 |
| TTQAPLRAAR | 552 |
| RPFRTRMTW | 553 |
| SPSMGAMRW | 554 |
| CQPALLEQLF | 555 |
| VSLPPPPLQR | 556 |
| ATQEVQMRSR | 557 |
| SIFPFQKTL | 558 |
| SPHLPNPTW | 559 |
| SVSWSIPRR | 560 |
| LDTMSCPPWK | 561 |
| RAGGMAPAK | 562 |
| MASTVTEVLR | 563 |
| TLQPASGSK | 564 |
| RSRAPRTATR | 565 |
| VAPTPQRPL | 566 |
| HPLVATQAL | 567 |
| QVWTAATLR | 568 |
| LSASASTSL | 569 |
| CAAWSSTRPW | 570 |
| SAAAPRHSR | 571 |
| FMQNIRIPI | 572 |
| RKSHRFASGK | 573 |
| STRPWTTSR | 574 |
| TAAMGPHTY | 575 |
| MTVKNLIQF | 576 |
| LPQHAPHTLLY | 577 |
| SMATVARAPR | 578 |
| CLGLWCSPR | 579 |
| QFFEIKSR | 580 |
| WRPSVGLFSM | 581 |
| HGQPVLSHR | 582 |
| APSWSSRRW | 583 |
| FSVMIPPM | 584 |
| SPWTSRRGPPW | 585 |
| ALRSPFLQGR | 586 |
| KMERFWKLIR | 587 |
| MNMKMKTFMK | 588 |
| KGATYTPRHPR | 589 |
| SSPTHVRAA | 590 |
| KMWEPPMVL | 591 |
| CQKKLMLLRLNLRKM | 592 |
| DSQHVNLFLTKMMRM | 593 |
| TYKVKAAASAHPLQM | 594 |
| QLFVMSDTTYKIYWT | 595 |
| EPVLSSLTSLRIELL | 596 |
| RLIIQNLKSVRAYLQ | 597 |
| LGGLIKLKLNRLNLI | 598 |
| IISLFITKAYTLERK | 599 |
| SILLHLIVLNSPESN | 600 |
| SFLFLRQRATSTITI | 601 |
| ELGILTSFGVQQKPR | 602 |
| GNINLTFFTTKKSMI | 603 |
| QARLCLIVSRTLLLV | 604 |
| VSFWTLLPTTGVRTR | 605 |
| RSLTCVLSVGRPLAT | 606 |
| RPRLLLARTSQELAV | 607 |
| GKLMHVLYFSSNEVT | 608 |
| LOEWSRAISGGIAKR | 609 |
| RGPLTSAPSAQQSLT | 610 |
| KKNLCLLKSGQLWML | 611 |
| KTEIQLTMNDSKHKL | 612 |
| QKGILMLQNCHSYHL | 613 |
| PGMLFFFATWSALVR | 614 |
| KKKGLMTLSKMIKKK | 615 |
| DQKILLLLEEKMIFR | 616 |
| GTALIVHMTITKGKE | 617 |
| VKPRKLTKVRFIKTL | 618 |
| LIWKRVFILLLSDKK | 619 |
| IVHFLLFKTSGRVQH | 620 |
| PLRISLSLSNLSNSP | 621 |
| VAWFYKSLWAPLLQR | 622 |
| EKKLKKTPSLQTHQS | 623 |
| MYSIRMENSTWMRPW | 624 |
| GKNGILEDSQKKRRY | 625 |
| WLKKKMLKNVRGRRK | 626 |
| GSFRFFGSRMSVLSFSN | 627 |
| VGKLVTLRNVSTKKY | 628 |
| RMQLCTQLARFFPIT | 629 |
| GTFMVIDCLSLRKKA | 630 |
| LKTLFHLRGISGNLK | 631 |
| IWNRIEPASTYRQYS | 632 |
| FWQICHIKKHFQTHK | 633 |
| NEWVKSDQVKKRKKR | 634 |
| SIPWAPTSSRSVCPI | 635 |
| ELRHVVPAPAHRGAL | 636 |
| WGFLHLVSPSGTQYF | 637 |
| LPTLRPTRPLQTVPL | 638 |
| PAAYLLGLPGVQWLG | 639 |
| LFFFVRQWGVQRVST | 640 |
| SKMYTTSMAMPILPL | 641 |
| DIGEVSQFLTEGIMK | 642 |
| PLMIKNRISPLLTIL | 643 |
| YFINFIYLAKSTKKP | 644 |
| VLLQMFLRGLKRLLQ | 645 |
| LWELRFHQDRGQRLP | 646 |
| RPLKCMATLPNIRKF | 647 |
| KEKKKNLAIVEEEMFL | 648 |
| EPGVLAAAAETEHL | 649 |
| YKKKAAICTTPALVK | 650 |
| TLETLVKDLKKKSRV | 651 |
| HVSVYPKRSFLCSSI | 652 |
| RIRKKKVKSSVLQLK | 653 |
| QPRLMILCSCLTMKE | 654 |
| RIAFGMMSMASRCPA | 655 |
| GLFYLLFCSKATECK | 656 |
| LVQSVLSSRGVAQTR | 657 |
| FPLAEKVKALADPSAFV | 658 |
| AWHFSRAATEVTWWA | 659 |
| RNSEITMQQIAVLLL | 660 |
| LSLIMLAQAQEVFFF | 661 |
| QEYLPLEFHKGYLLS | 662 |
| VTNFLKATGLNIWKL | 663 |
| GFELLRKNGLERRTR | 664 |
| FSGICYLLSSVSFFL | 665 |
| PGILQQKMQFRLLVL | 666 |
| ATTIRLCWKASGRLA | 667 |
| QRASALLASCSKKLH | 668 |
| ESAVTALVPPPALSL | 669 |
| ALPRIHNMSKAALRV | 670 |
| ELQLSVLSAESLRENFS | 671 |
| VSGWVVVKSEPIGPL | 672 |
| RHRLNDIMTALLVQK | 673 |
| PRRKTWKMRKKYAQK | 674 |
| YLIKLLSRDLAKKKL | 675 |
| PYLNTTGYPAPLHQE | 676 |
| QFVISPPALRSRQKT | 677 |
| QLGSGSSEASSVPHL | 678 |
| PYFATYKAKMPLLRW | 679 |
| FCKIKVSSAILSKKT | 680 |
| AKVLLVRLKKNRSYL | 681 |
| TAVTVMAGSVPSAQSV | 682 |
| FYLGYNAMQDFFSYY | 683 |
| NLLLKDQKPKKTRKI | 684 |
| QDTVIIKKNPALPKH | 685 |
| AGWVGKWAGLQLPFY | 686 |
| LMLQHITLMCSAYRN | 687 |
| KKKRKIIEFRIKFLF | 688 |
| FLDIHNIHVMRESLKKS | 689 |
| LRVLVLMNSKHTFLA | 690 |
| ECIEVYGYHNIRVYK | 691 |
| CFFFFCYILNTMFDR | 692 |
| APLRLWSWCGTSQTY | 693 |
| TRLMAPVGSVMSCSL | 694 |
| FFFSVIFSTRCLTDS | 695 |
| KKRKIIEFRIKFLF | 696 |
| QVPALAPQAWWPARR | 697 |
| EFLSHPFAVTLYGGG | 698 |
| MSVCFFFLLYSQHDV | 699 |
| PSQVWTAATLRCPAV | 700 |
| RGRMLTWRSSLWLQG | 701 |
| PQSLRPVRVRAHPGL | 702 |
| RSKFTGLCRPLTSLA | 703 |
| RSSTTAGLPARCAAW | 704 |
| RGEAMGSGQATVTSMS | 705 |
| NHIVVSAEGNISKKQ | 706 |
| IPPLHLRTSARSKFT | 707 |
| GEAMGSGQATVTSMSP | 708 |
| TNMEIPHFFVILPKS | 709 |
| KKKTYTCAITTVKAT | 710 |
| NIDLCTALSALSGIP | 711 |
| VPQLLHLPQFHSLRR | 712 |
| SSLITMLTPRQKARK | 713 |
| LQGFIQDRAGRMGGR | 714 |
| REKIINPTISCPFQS | 715 |
| VGTMSSSWRLWNKTR | 716 |
| QTPASLMITRARKGR | 717 |
| SPLPRGSGAAPLSWDS | 718 |
| KTYTCAITTVKATETK | 719 |
| QLLDLKSSLLKRPIH | 720 |
| FRQDKLKVHMRKHTG | 721 |
| VQSLLMYKDGDSVLQRGA | 722 |
| IKGSESATYVPVAPPH | 723 |
| EETQEKMTILQTYFR | 724 |
| MLGNVESGGPHLLQPA | 725 |
| ENALLNGSSFLVSSSI | 726 |
| RPFFLPVYRQTHWRL | 727 |
| MCTWLTMAPSCLRRS | 728 |
| TLPVQRLRALSQNER | 729 |
| PPAALRSGIPRLLHQ | 730 |
| SGWRRLHRAMPSSGA | 731 |
| PPPLQRLQGHLGRPY | 732 |
| QAPFFCLRVRCGGWL | 733 |
| VWMASASVTASTAGMT | 734 |
| LVAISFKTVFKASHA | 735 |
| NVTFLMQATLCARQE | 736 |
| ASITSISRTTSSLSS | 737 |
| QVIVSRGGALSGSPRL | 738 |
| GVQGPSMATVARAPRA | 739 |
| ESTPTASSAMAVTRSS | 740 |
| TTLWLGPASVAPATLP | 741 |
| SDGTPGGAPAQPACPR | 742 |
| RLQGLAGAPRGRRSA | 743 |
| GPELRRSRAPRTATR | 744 |
| PSTAVATWRRCAGPA | 745 |
| AMMNGKVPFFSALKV | 746 |
| PLTSGGSAAATAKWG | 747 |
| HTGTQFFEIKSRPLT | 748 |
| LGQILILPPWAFLDP | 749 |
| RSWRFLRAAPSSSAR | 750 |
| GPQLLLSHRAVPHVH | 751 |
| TNLRVQLLKRQLSLS | 752 |
| QGEVDSQQGARAAAE | 753 |
| AILLQVIAKKMTSTL | 754 |
| LEQLFLCALKARAFR | 755 |
| QERVRLIPRLRSLPR | 756 |
| SPSTPGAATAAASSRP | 757 |
| RTFCLTARRGALAIR | 758 |
| EEVVCLLFPASGEMK | 759 |
| RRRATLSAAFARRRF | 760 |
| STLPRPFRTRMTWRS | 761 |
| PMFLALDRRGGPGQA | 762 |
| PTCLLLSRPLRSPHL | 763 |
| LREWTTQAPLRAARW | 764 |
| FLPWVPERGVASWTW | 765 |
| VQMRSRMSSSARRMW | 766 |
| GPLVPGLVLGGVREEE | 767 |
| LPALSILQRSPRLPR | 768 |
| AQSSWRSLEASALPV | 769 |
| PESFKRQARARLERR | 770 |
| SPVTQITGAAARQLL | 771 |
| YAGGVGAQLMAPLSP | 772 |
| VTELAQVIVSRGGGA | 773 |
| PTHVRAAARVSSTAS | 774 |
| RRPCCWMGAAAVWRG | 775 |
| HDLGLHVLSCRIIPV | 776 |
| WKAKREKMRAKQNPPGPAPPGGGSSDAAGKPPAGALGTPA | 777 |
| QYQEEAEEEVQEDLTEEKKRELEHNAEETYGENDENTDDK | 778 |
| GNKCTMCKEKLEREAAEKKKKEDEDRSNTGERSNTGERSN | 779 |
| SKLRLAPDKLKSTESELKKKEKRRDEMLGLVPMRQSIIDL | 780 |
| RGEGRFGVSRRRHNSSDGFFNNGPLRTAGDSWHQPSLFRH | 781 |
| DLINKFGTLNGFQILHDRFFNGSALNIQIIAALIKPFGQC | 782 |
| PVKVLVGKNFEDVAFDEKKNVFVEFYAPWCGHCKQLAPIW | 783 |
| FNLQKSSLSARHPQRKRRGGPSEPTPGSRPQDATVHPACQ | 784 |
| CAGPQTYKEHLEGQKHKKKEAALKASQNTSSSNSSTRGTQ | 785 |
| DLPIDDKLDNQCVSVEPKKKEQENKTLVLSDKHSPQKKST | 786 |
| NFSVRCPKHKPPLPCPLPPLQNKTAKGSLSTEQSERG | 787 |
| ALFGLDRQTLWCKPCRRKKKCVRYIQGEGSCLSPPSSDGS | 788 |
| DQLQQAVQSQGFINYCQKKIDASQTEFEKNVWSFLKVNFE | 789 |
| LAEPPHFVEHIRSTLMFLKKHPSPAHTLFSGNKALLYKKN | 790 |
| LSSSGFLDASDPALQPPGGVPSSLAESHLCLPSAFALSIP | 791 |
| PTVALSAVAGASQVKWNKKNANCLATSHDGDVRIWDKRKP | 792 |
| GFGLGKVSYIGVCQSKFHFFEDQLRGAGFGPQHNRHCLLT | 793 |
| SKVRGISEVLARRHMKVAFFGRTSNGKSTVINAMLWDKVL | 794 |
| EPGPLGGGGSGGPQMGLPPPPPALRPRLVFHTQLAHGSPT | 795 |
| EEAYRCNFLGLSPHVQIPPHVLSSEFAVIVEVHAAARSTL | 796 |
| MSYAANLKNVMNMQNRQKKEGEEQPVLPEETESSKPGPSA | 797 |
| HKDAWRQPEDTWAALEGLSFSPFREQMLDTSSLLQFMREK | 798 |
| IIYNQGFEIVLNDYKWFAFFKYKEEGSKVTTYCNETMTGW | 799 |
| LKSQKQVKVEMSGPVTVLTRQTTAAELDSHTPALEQQTTS | 800 |
| DEAFDTANSSIVSGESIRFFVNVNLEMQATNTENEATSGG | 801 |
| KIREVRQKIMQAATPTEQPPGAEAPLPVPPPTGTAAAPAP | 802 |
| EINVIIKNPEIVFVADMTKNDAPALVITTQCEICYKGNLE | 803 |
| ATYVTFSPNGTELLVNMGGEQVYLFDLTYKQRPYTFLLPR | 804 |
| PLMILDEERELEKLFQLGPPSPVKMPSPPWESNLLQSPSS | 805 |
| KSKGPKKTAKSKKKKPLKKKPTPVLLPQSKQQKQKQANGV | 806 |
| RNLAFFQLRKVWGQVWHSIQTLKEDCNRLQQGQRAAMMNL | 807 |
| PKAARIKEVLKERKVLEKKVALSKKRKKDSRNVEENSKKK | 808 |
| LKQKFSMKAQNGFNKKRKKNVFNPKRVVEDSEYDSGSDVG | 809 |
| IIKCIEDIKRPGEWSGLEKNKKDGFKSSQLNNPQFVWVVP | 810 |
| EKVASDTEEADRTSSKKTKTQEISRPNSPSEGEGESSDSR | 811 |
| TANMKASENLKHIVNHDDVFEESEELSSDEEMKMAEMRPP | 812 |
| SCLIKYNVSTTPYLQSVKKKVQFDGTNSAFKELKFLTPVR | 813 |
| LFKPADVILDPDTANAILLVSEDQRSVQRAEEPRDLPDNP | 814 |
| SMVDEVSGKVLEMDISKKKALQQKDIHKKIKQNESATDEI | 815 |
| VQERKIPAHRVVLAAASHFFNLMFTTNMLESKSFEVELKD | 816 |
| SFGSPTGNQMSSDIDEYKKNIHGNALRTSGSSSSDVTKAS | 817 |
| IIFNFEKAYFILDEFLMGGDVQDTSKKSVLKAIEQADLLQ | 818 |
| EDSPYETLHSFISNAVAPFFKSYIRESGKADRDGDKMAPS | 819 |
| GWGPPPPPPPLLPCTCSPPVAGGMEEVIVAQVDHGLGSAW | 820 |
| KKFKEEKKLKAKLKKVKKKRRRDEELSSEESPRRHHHQTK | 821 |
| CSIERADNDKEYLVLTLTKNDLDKANKDKANRYFSPNFKV | 822 |
| YATNPPWIFTQEAPEEGTGGFDGIYYGDNRFNTVSESGTA | 823 |
| FTKTPKSSSPALKPKPNPPSPENTASSAPVDWRDPSQMEK | 824 |
| MPSVCLLLLLFLAVGGALGNRPFRAFVVTDTTLTHLA | 825 |
| GVYSRYFTTYDTNGRYSVKVRALGGVNAARRRVIPQQSGA | 826 |
| DVKEETKEWLKNRIIAKKKDGGAQLLFRPLLNKYEQETLE | 827 |
| LLDETSAITLTSTWKEVKKIIKEDPRCIKFSSSDRKKQRE | 828 |
| VHSSSEPLRNLHLDIGALGGDFEYEESLRTSQPEEKKDVS | 829 |
| STSYLLCISENKENVRDKKKGNIFIGIVGVQPATGEVVFD | 830 |
| AQRKSSMNQLQQWVNLRRGVPPPEDLRSPSRFYPVSRRVP | 831 |
| KLDQGEYERAAIDAVDNKKNTPLHYAAASGMKACVELLVK | 832 |
| EKEERRVWTMPPMAVALKPVLQQSREARDELPGAPPVLCS | 833 |
| ACGETLSVTSEENSLVKKKERSLSSGSNFCSEQKTSGIIN | 834 |
| NPQRAQLCEDHCVDGCFCPPGTVLDDITHSGCLPLGQCPC | 835 |
| SLRQQEERKRLYQRQQERGGIIDLEAERNRYFISLQQPPA | 836 |
| SSKHNVIVGRNGSGKSNFFYAIQFVLSDEFSHLRPEQRLA | 837 |
| MHGGGPPSGDSACPLRTIKRVQFGVLSP | 838 |
| NQTTQEADSANTLQIAEIKEKIGTRSAEDPVSEVPAVSQH | 839 |
| EREAGGPLPPSPLPHSSPPTAAVATTSITTATPGVPGLPS | 840 |
| KAYLKQAPPSKGPTVRTKKVGKNEAVLEWDQLPVDVQNGF | 841 |
| PISIIDQGEPKGTGATCGKKGSQAGAEGQPSTVKRYTPAR | 842 |
| GSRLGNSLLLKYTEKLQEPPASAVREAADKEEPPSKKKRV | 843 |
| EGGNLPDAAEPDIISTIKKTFNFGQNEALHLFQTLMECMK | 844 |
| PPPEDSPMSPPPEESPMSPPPEVSRLSPLPVVSRLSPPPE | 845 |
| FNYNRAFQVWAVPLLLVAFFAYLVAHSFLSVFETVLDALF | 846 |
| SDEKRLCLQLLSDVLRGQGEAGQLEEAFSLALLPQLVVSL | 847 |
| RKEEAFRHKLAMDTYSGPPPGPGPGPALPAHSSPGLPPPA | 848 |
| REEKEKLFNEHIEALTKKKREHFRQLLDETSAITLTSTWK | 849 |
| PYPQGGYPQGPYPQSPFPPNPYGQPQVFPGQDPDSPQHGN | 850 |
| RGLAQADGTLITCVDSGILRVWHDKDKDTSSDPLLELRVG | 851 |
| SIKYNEEKRHVDQPIDYSLKYATDIPSSQKQSFSFSKSSS | 852 |
| QVMKYILDKIDKEEKQAAKKRKREESVEQKRSKQNATKLS | 853 |
| RLESFLLQTGYAAGKGVGGGSADLIRNLRSRVDPQAPDLP | 854 |
| DGKFANLTPSRTVPDSEAPPGWDRADSGPTQPPLSLSPAP | 855 |
| DGTFSVTSAYSSAPDGSPPPAPLPASEMTMEDMAPGQLSS | 856 |
| GGPQDPQPGLTAHVVSAGGRAEMHCFSIMVTPDPSTPSRL | 857 |
| KLDYAVAWFIRESMTIYIFLSALWDPTISWRTGRYRLRCG | 858 |
| VNQEVLEILDFHLYGSYPPGTPALKAYWENTYDAADGPSG | 859 |
| MAVGASIAARLGTYPDWFFFCSFIGMFVFYCAHWQTYVSG | 860 |
| RYPRKKFWVGKPIARVVKKKTGEFSDKLLSLQRGLREFQG | 861 |
| LPPVFGEEYEEQPRPRSKKKGAKRKAVSGYQSHDDSSDNS | 862 |
| FRQRKAQSDGQSPSKKQKKKRKTSSSKHDVSAHHDLNIDQ | 863 |
| QVFALLFVTEYLTKWPKFFFDILSVVDLNPRGVDLYLRIL | 864 |
| TILYFPFSSHSSYTVRSKKIFLSKLIVCFLSTWLPFVLLQ | 865 |
| LNIKKISEEEYVALGSFFFWKCLHGESSTEDMCHTLESAG | 866 |
| PYHWSPSRKAGRSDSSSSGGGGSPSEASGLGLDFEDSVWK | 867 |
| GASLYVGWAASGLLLLGGGLLCCNCPPRTDKPYSAKYSAA | 868 |
| PGPCGPPPGHGPGPCGPPPHHGPGPCGPPPGHGPGHPPPG | 869 |
| ESHSLSAHLQLTFTGFFHKNDKPSPNSENEQNSVTLEVLL | 870 |
| VPNTDQKSTSVKKDNHKKKTVKMLEYLGKDVLHGVFNYLA | 871 |
| QLKEMCRRELDKAESEIKKNSSIIGDYKQICSQLSERLEK | 872 |
| QTEMRVQLLQDLQDFFRKKAEIETEYSRNLEKLAERFMAK | 873 |
| DVLFVYAVRECCKCIDGKKVGKELTEKPKFILSVLLLWNF | 874 |
| TVYEFLLMKVEKDHLAKPFFPAIYKEFEELHKMVKKMCQD | 875 |
| CESKLYSLDHGHEKPQDKKKRTSGLATLKKKFIKRRKSNR | 876 |
| KGHYSLHFDAFHHPLGDTPPALPARTLRKSPLHPIPASPT | 877 |
| PLGQSHLAHHSMAPYPFPPNPDMNPELRKALLQDSAPQPA | 878 |
| KNISSEHSMSSTPLTIGEKNRNSINYERQQAQARIPSPET | 879 |
| EPNASVVPPPLPATWMRPPREPAQPPREEVRKSFVESVEE | 880 |
| SYAPAEIFLPKGRSNSKKKRQKKQNTSCSKNRGRTTAHTK | 881 |
| SSSGAFSLLGRFCGAEPPPHLVSSHHELAVLFRTDHGISS | 882 |
| TIMNRRLCCILVALSWMGGFIHSIIQVALIVRLPFCGPNE | 883 |
| HDFILEDAASPKCIMKEKKKPGETFFMCSCSSDECNDNII | 884 |
| PVAVPAPQQADGNPDVPPPRPLQGRSEREFFVKWVGLSYW | 885 |
| LGRVQEFDSGLLHWRIGGGDTTEHIQTHFESKTELLPSRP | 886 |
| GGNESQPDSQEDPREVLKKTLEFCLSRENLASDMYLISQM | 887 |
| PKAEDGATPSPSNETPKKKKKRFSFKKSFKLSGFSFKKNK | 888 |
| EIGQHPSLEDMQEVVVHKKKRPVLRDYWQKHAGMAMLCET | 889 |
| ATIMNQRLCCILVALSWRGGFIHSIIQVALIVRLPFCGPN | 890 |
| HIRKQQMVSVEETIYIVGGCLHELGPNRRSSQSEDMLTVQ | 891 |
| SDFDRVGGMNTEEFRDQWGGEDWELLDRVLQAGLEVERLR | 892 |
| TGIFRLTNAGMLEVSACKKKGFHPHTKEPRLFSICKHVLV | 893 |
| VPRVEGVFIFLIEDSGKKKRRKNFEAMFKGILQSGLDNFV | 894 |
| NKHAAFSCPKKPLSPPKKKVSHSSKKGGHSSPASSDKNSN | 895 |
| GENARLHYRLVDTASTFLGGGSAGPKNPAPTPDFPFQIHN | 896 |
| ESKFKSRASNAQAKPSSFFLQMQKRVSGHYVTSAAAKSVH | 897 |
| SSQLLTPAERPGGLDDRSPPGSSETVELVRYEPDLLRLLG | 898 |
| NCHISLTPNGDMPGSEIPPSSPSHAGSLHDDLNQVSRDDA | 899 |
| ESIGDYYACARLSCAPPPPIHAINRGIFVEGLSCVLDGIF | 900 |
| DLGLCVAELELLSSWFSPPTVVAGRRKSVDQPEGTPVELY | 901 |
| TLEILKSTMKKELEAAQKKKPSLCEMLHMPNICKRISLLS | 902 |
| PCFYPDEDDFYFGGPDSTPPGEDIWKKFELLPTPPLSPSR | 903 |
| TGALLLQGFIQDRAGRMGGEAPELALDPVPQDASTKKLSE | 904 |
| STSRSRPSRIPQPVRHHPPVLVSSAASSQAEADKMSGTST | 905 |
| NQEFLQARTPTLASTPIPPTPQAPSPAVDAEIRAQDAPLS | 906 |
| GAGPSGETGAAGVDGGCGGRH | 907 |
| GQEQTFGILNVLEFSSDRKRMSVIVRTPSGRLRLYCKGAD | 908 |
| ETMKQARHRLASFKTVIKKKGSVFPDDGRKSFLTREEVLS | 909 |
| DFFRAQTLKETSLTNTMGGYKESFSSIMCFGCHDVYSQAS | 910 |
| GRCKSGFDPRHGSHNIKKKAWYLIAMLLKLAFCLALCAKL | 911 |
| DGLRSRVKYGVKTTPESPPYSSGSYDSIKTEVSGCPEDLT | 912 |
| SIRTTDFHNPGYPKYLGTPHLELYLSDSLRNLNKERQFHF | 913 |
| REPAGLSLVLKKIPIPETPPQTPPQVLDSPHQRSPSLSLA | 914 |
| PQGLPGVKGDKGSPGKTGPRGKVGDPGVAGLPGEKGEKGE | 915 |
| TAPSLQNNQPVEFNHAINYVNKIKNRFQGQPDIYKAFLEI | 916 |
| AVSTGVQAGIPMPCFTTALSFYDGYRHEMLPASLIQAQRD | 917 |
| CTHCYCLQGQTLCSTVSCPPLPCVEPINVEGSCCPMCPEM | 918 |
| GTMRATGDFVTVKDGEIFFLGRKDSQIKRHGKRLNIELVQ | 919 |
| LSLEINRKLQAVLEDTLLKNITLKENLQTLGTEIERLIKH | 920 |
| PMAFSPQRDRFQAEGSLKKNEQNFKLAGVKKDIEKLYEAV | 921 |
| LTDRQVKIWFQNRRMKEKKINRDRLQYYSANPLL | 922 |
| GSAAAGGPTSYGTLKEPSGGGGTALLNKENKFRDRSFSEN | 923 |
| GRVGPRRQRKHCITEDTPPTSLYIEGLDSKEAGGQSSQEE | 924 |
| GAKIQWLKDAQGLPGGGGGDNSGTAENGRHSDLAALYTIV | 925 |
| VRIAAPGIGVWNPAFDVTPHDLITGGIITELGVFAPEELR | 926 |
| FRSKQEALKQGWLHKKGGGSSTLSRRNWKKRWFVLRQSKL | 927 |
| EHVENAGVHSGDATLVTPPQDITAKTLERIKAIVHAVGQE | 928 |
| QNYTNWSTSPYFLEHGIPPSCCMNETDCNPQDLHNLTVAA | 929 |
| PTGPKNMQTSGRLSNVAPPCILRKNPPSARNGGHETDAQI | 930 |
| DFLSVKWEAAMMNGKVPFFFSSESLGYFATGRPADNVMTT | 931 |
| GCGFCNQDRRTLPGGQPPPRVFLAVFVEQPTPFLPRFLQR | 932 |
| VLQTGTQRTIAPRTHPYSPKIDGTRTPRDERRRAQHNEVE | 933 |
| AMDRNSLQSAVSNIAQAPLFIPPNSDPVPARDYLILKGVL | 934 |
| DSCLLAAMAYDCYVAIRHPLPYATRMSRAMCAALVGMAWL | 935 |
| GLVYLIYYEESLHHPMYFFFGHALSLIDLLTCTTTLPNAL | 936 |
| MSYFPILFFFFLKRCPSYTEPQNLTGVSEFL | 937 |
| DSANAKTLLEAASKFQFHTFCKVCVSFLEKQLTASNCLGV | 938 |
| LWENETVGAQDDPLAYWEKKREAWPPSICLTPHRSLL | 939 |
| SDPSQQPPSYGGPSVPGSGGPPAGGSGFGRGQNHNVQGFH | 940 |
| PGSRPKKKLSPPSITIDPPESQGPRTPPSPGICLRRRAPS | 941 |
| MSGQDVIKAVEDGFRLPPPRNCPNLLHRLMLDCWQKDPGE | 942 |
| AVVGIEDPVRPEVPDAIKKCQRAGITVRMVTGDNINTARA | 943 |
| GLPSKIGSISRQSSLSEKKIPEPSPVTRRKAYEKAEKSKA | 944 |
| MAERESGGLGGGAASPPAASPFLGLHIASPPNF | 945 |
| QGIAVLNIPSYAGGTNFWGGTKEDDTFAAPSFDDKILEVV | 946 |
| APRFSRGLRGELSYNLGAGGGSAEVDTSSLSGDNTLMSTL | 947 |
| QCYQEVCNDRINANTITSPRLAALTYKCTRDQWTVYCRVI | 948 |
| IIIKCLLYARHGVLFLFFF | 949 |
| RTAPLGVPGTLPGLPRRDPLRVALRLDAACWEWARSGCAR | 950 |
| HKTLLERHVALHSASNGTPPAGTPPGARAGPPGVVACTEG | 951 |
| RLGSVMRPTEDITARELPPPTSAQGPGRVGPRRQRKHCIT | 952 |
| SLRRHYEVHHGLCILKEAPPEEEACGDSPHAHESAGQPPP | 953 |
| RGACPGLLETLGALRAIPPAQLQEEAFMSQVHSVVLSERD | 954 |
| AHLRKAEREEKPKHTEAKKSLSFRKKQQKDFCFIFRN | 955 |
| AGQGCKDALQLLIEHSWERGERLDMQALKQSSTELLFGGH | 956 |
| SCGPGTQHRQLQCRQEFGGGGSSVPPERCGHLPRPNITQS | 957 |
| SSSLSSGHVHSTPFQAGTPRYDVPIDMSYDSYPHHGIGTQ | 958 |
| ASTVEGGDTALLPEFPRGPLDAYRARASFSWKELALFTEG | 959 |
| ARRGMHAFIVPIRSLQDHTPLPGIIIGDIGPKMDFDQTDN | 960 |
| MVTQILGAMESQVGGGPAGPALPNGPLLGTNGATDD | 961 |
| YGRDRGIFGIESWPDYEDIYKKTIEVGTFLDLPRFPDITE | 962 |
| MNQENRSSFFWLLVIFTFLLKITASFSMSAY | 963 |
| STLRGRARAMSKASKVPGGVQARLEKDAAAPALEDLPWTS | 964 |
| LKKFKMHLEDYPPQKGCIPLPRGQTEKADHVDLATLMIDF | 965 |
| GLKTKKWVNEVRYGGFSLGGRDPGLPSGQELGRSVEELWA | 966 |
| GHQEGLVELPASFRELLTFFCTNATIHGAIRLVCSRGNRL | 967 |
| GGGAEARGATAGASACQGGLYGGVAGVAYMLYHVSQSPLF | 968 |
| DVECHLLTHVPMWSARLLTCPCGVPACSHVPMRSARLLTR | 969 |
| EEKPGRKRAEAKGNRSWSEESLKPSDNEQGLPVFSGSPPM | 970 |
| SLASPMRLSTPSASPAIPPLVHCADKSLPWKMGVSPGNPV | 971 |
| LAQPPKDLTLELAGSPSVPLVIGCAVSCMALLTLLAIYAA | 972 |
| DRNNSSCRNYNKQASEQNWANYSAEQNRMGQAGSTISNSH | 973 |
| DGYTGEHCEVSARSGRCTPGVCKNGGTCVNLLVGGFKCDC | 974 |
| SELKEARQRRQPPGHRPAPRGGGGSALVRGSPCVESCWAP | 975 |
| MAAKTPSSEESGLPKLPVPPLQQTLATYLQCMRHLVSEEQ | 976 |
| SYTVENAYECSECGKAFKKKFHFIRHEKNHTRKKPFECND | 977 |
| TYPPSASVVGASVGGHRHPPGGGGGQRSLSPGGAALGYRD | 978 |
| ADKPASLPPASQASNHRDPRQARRLATETGEGEGEPLSRL | 979 |
| TAQLKTTPTQPSEQKAAFPPPEQKTAFDKKLLDRFDYDDE | 980 |
| KKERTSIFEMSDFSCVGKKTRTVDITNFTAKTISSPRKTG | 981 |
| LGNPFGLIREFSEGVEAFFYEPYQGAIQGPEEFVEGMALG | 982 |
| ESRHSLEERLQQIREDEEREGSELTLNSREGAPTQHPLSL | 983 |
| RLREEEKRRRREEERCKKKETDKQKKIAEKEVRIKLLKKP | 984 |
| SQQPPSYGGPSVPGSGGPPAGGSGFGRGQNHNVQGFHPYR | 985 |
| ALVVLDVHARDVLSSLVKKNISDDSDFEWLSQLRYYWQEN | 986 |
| AYIQSRQALNSVVKITSKKKHPELITFKYGNSSASGIEIL | 987 |
| VKVVQGPAGGDNSKLRYKKKGSHCLEVTVQ | 988 |
| DNTWSITCPLCRKVTAVPGGLICSLRDHEAVVGQLAQPCT | 989 |
| RWTEMIRASRENPAMQEKKRSSIWQFFSRLFSSSSNTTKK | 990 |
| WVSQFVTFYPGDVILTGTPPGVGVFRKPPVFLKKGDEVQC | 991 |
| AFVGLLASCLGLELSRCRAKPPGRACSNPSFLRFQLDFYQ | 992 |
| LVARALANECSQGDKKVAFFMRKGADCLSKWVGESERQLR | 993 |
| GVDVCFFGMHVQEYGSDCPPPNTRRVYISYLDSIHFFRPR | 994 |
| TASALLPKRAMQFGSRIAKMEKINEKASDKCGRLQIMSLE | 995 |
| PRGPKVGSLGLPAHPREKKTSKSSKIRSLADYRTEDSNAG | 996 |
| GKKYQWDAETQGWILGSFFYGYIITQIPGGYVASKIGGKM | 997 |
| ISLQDLSKERRPGGAGGPPIQDEDEGEEGPTEPPPAEPRT | 998 |
| MAAGCLLALTLTLFQSLLIGPSSEEPFPSAVTIK | 999 |
| VASHPETRSAFLAAHIPLFLYPFLHTVSKTRPFEYLRLTS | 1000 |
| CQKCGKAFSRASTLWKHKKTHTGEKPYKCKKM | 1001 |
| WDMAQLRAVVVDDYRRRKKKGGPLRALSSKTWSTDDFFAG | 1002 |
| IQMKSADRAFMAAQKCHKKNMKDRYVEVFQCSAEEMNFVL | 1003 |
| PAVPFSRSRQPSPLLLLPPPAGLTSDPGPSVRRVPAVQRD | 1004 |
| ESTQLQNEIQKLOKTLKKKTKRYMSHKLKI | 1005 |
| GLAMSSSIFIGGSFILKKKGLLRLARKGSMRAGQGGHAYL | 1006 |
| DLALRGKKKRKKFMKDAKKKGEMTAEERSQFEILKAQMFA | 1007 |
| NLLPYKIAYYIEGIENSVFTLSEGHSAQICTAQLGKARLH | 1008 |
| MKSCGVSLATAAAAAAAFGDEEKKMAAGKASGESEEA | 1009 |
| LRDSIQSAQELLAQEQKKKEELEIATSQLKSDLTSRDDLI | 1010 |
| PGCLPMVKRTITRQQWKKKALRSMPKSRNQVLFRRNLTPS | 1011 |
| EASASPPRSEAQRQIQEWGVSVRTLRGNFESASGPLCGFN | 1012 |
| TTTDMYLLILQHFLLHATPPDSASQGLGPSLLRGRLPTLL | 1013 |
| RGKPPPQAHLPSAPALPPPHPPVVLPHLQHSVAGHHLGPP | 1014 |
| LQLTSGVHSTIKVIKAKKKT | 1015 |
| VRQGALQGGLLMGYSPAGGATSPGVYQVSIFSPPAGTSEP | 1016 |
| HYSGGESHNSSSSKTFEKKRGKK | 1017 |
| SEHHLQRAISAQQVFREKKESMVIPVPEAESNVNYYNRLY | 1018 |
| KKRFKSLEKSHKNTGELKKSKVLSHHRAGRSNQIKIEQIK | 1019 |
| CDECGKTFIRHDHLTKHKKIHSGEKAHQCEECGKCFGRRD | 1020 |
| DQPSILNSCEDPVPGMLFFLPPGQHLSDYSQLNESTTKES | 1021 |
| RRKSKRMSKYKENKSENKKTVPQKKMHKSVSSNDAYNFNL | 1022 |
| VHDASTSSDSEEQDMSVKKGDDLLETNNPEPEKCQSVSSA | 1023 |
| ILTSLWLLEQPYFATYKAKNAIIKMVENRDTGCQIGPNIE | 1024 |
| KEQDRVHSPCPTSGSEKKKRSDDPVEDDKEKKELGYLTVE | 1025 |
| VPTIFSLPEDNQGKDPSKKKSQKKNLEDEKEVCPKAKSEE | 1026 |
| ELVEMCNGKNGILEDSQKKEDTAFSDWSDEDVPDRTEVTE | 1027 |
| EPQKSGNNETFTPNRVEKKKLQHTYLCEEKENNKSFQSDD | 1028 |
| LAGGRHCCPVCRWPSYKKKQPYAQHQPLSNDVPS | 1029 |
| KQNVKMSESQAALPSALKTLQQKLRLHIIEIIGNEGLLAC | 1030 |
| RPAERITEDHEKKSKRIKKN | 1031 |
| NYKVDCACHKGNRNCPIQKRNPNATELPLLPPPPSLPTIG | 1032 |
| NGSFYFSGICYLLSSVSFFFVPLAERWKNSLT | 1033 |
| VIEVGKKHGPWVNHYSIFFVSVSFFIITAATVGYFIFYSA | 1034 |
| PDEIGNFIDENLKAADTDPTAPPYDSLLVFDYEGSGSEAA | 1035 |
| TRWKEDIRYHYAEISSQVPLGKRLREYFNSEKPEGRIIMT | 1036 |
| VLDKVFRASESQILSIAEKMLDTRVAENRDLGMNENNIFE | 1037 |
| YRTSMFKTFKKTLDDGFFPFIILDAINDRVRHFDQFWSAA | 1038 |
| KEKYQEEFEHFQQELDKKKEEFQKGHPDLQGQPAEEIFES | 1039 |
| SLNRITDIGEVSQFLTEGIIMKDFSHPNVLSLLGICLRSE | 1040 |
| ASSSSSSSSSSSRSRSRSLSPPHKRWRRSSCSSSGRSRRC | 1041 |
| ERQMAELMPVGDNNFSDSEEGEGLEESADIKGEPHGLENM | 1042 |
| DPKPALRWGDSKGSNCQGGWEDDSAATGMVKSNQWGNCKE | 1043 |
| KPQIKQVVPEFVNASADAGGSSATYMDQAPSPAVCPQAPY | 1044 |
| KWCQRKLQAELKIGSFRFFWIQNVSLKFQQHQQTVEIDNL | 1045 |
| DPNFGSKEDFDSLLQSAKKKSIRVILDLTPNYRGENSWFS | 1046 |
| SILYYLGLVQWVVQKVAWFLQITMGTTATETLAVAGNIFV | 1047 |
| SNVNRSLSGDQDTFHPSGGSHTTHGSESDGHSHGSQEGGA | 1048 |
| RKEEREIKDEKYIDNLEKKQWITKWNENESYS | 1049 |
| ELSPLLMILSQLLPQQRHGERTPYVLRCLTEVALCQDKRS | 1050 |
| QAAAVQKRVETVSQTLRKKNKQYQIPDVRDIFAQQRESKE | 1051 |
| KLHCKQDGEEGTEEDTEEKCTICLSILEEGEDVRRLPCMH | 1052 |
| MTELEPSKFSKQAAENEKKYYIEKLFERYGENGRLSFFGL | 1053 |
| SSPSPGQQVQTPQSMPPPPQPSPQPGQPSSQPNSNVSSGP | 1054 |
| KPDAEYPEWLFEMNLGPPKTLEELDPESREYWRRLRKQNI | 1055 |
| QMVAFLEQRASALLASCSKNCTNSPAIVRFSGQSRGVPAV | 1056 |
| WWFWPLCCKVVIKDPPPPPAPAPKEEEEEPLPTKKWPTVD | 1057 |
| IKDKLKCYDFDVHTMKTLKNIISPPWDFREFEVEKQTAEE | 1058 |
| IHTGEKPYECSDCGKSFTKKSQLQVHQRIHTGEKPYVCAE | 1059 |
| WRKLVSKTQLEMNLPLMIKKQDQPTFDNSGNILSKEEKAT | 1060 |
| RQRELQLSVLSAESLRENFFLGGVTLPLKDFNLSKETVKW | 1061 |
| ESHNFSGDIALLELQHSIPLGPNVLPVCLPDNETLYRSGL | 1062 |
| LSPVDCIPEENNSAHPSFFSSSSKGDSFAQH | 1063 |
| LKIEETNPSLAQDTVIIKKKSCSSKALNTPVLSVLKEAAK | 1064 |
| AIARLDNSAAKHKLAVKPKKQRVSKKHRRLAQDPQHEQGG | 1065 |
| DAQRFMDLATYINMIWSAPLQVILALYLLWLNLGPSVLAG | 1066 |
| AAVCPTDLPQLWKGEGAPGQPAEDSVKQEGLDLTGTAATA | 1067 |
| LALSVETDYTFPLAEKVKAFLADPSAFVAAAPVAAATTAA | 1068 |
| QKYQKKEKKKEKKSKSKKGKHHKKEKKKRKKEKHSSTPNS | 1069 |
| TVFLTAILGGTIVIVIGFFAVLLCYCRDKCGTPQKRERNI | 1070 |
| LSAQMLAPPPPGLPRLALPPATKPATTSEGGATSPTSPSY | 1071 |
| LNFGTVSFYLGYNAMQDFFPTMSMKPNPQCDDRNCRKQQE | 1072 |
| VLVHKKKDCPPGSFWWLIPLLLLLLPLLALLLLLCWKYCA | 1073 |
| PNLMPSNPDSGMYSPSRYPPQQQQQQQQRHDSYGNQFSTQ | 1074 |
| LCNVTLNSAQQAQAHYQGKNHGKKLRNYYAANSCPPPARM | 1075 |
| VQKAEALMRELDEEGSDPPLPGRAQRIRQVLQLLS | 1076 |
| SDLSSQFVISPPALRSRQKNTSNKNKLEDELKDDAQSVET | 1077 |
| NSPTWSLQVFSKKKKKKKKNNMAAKEKLEAVLNVALRVPS | 1078 |
| YLESSLISHESAVTALVPPGSESFDILTAGIQATSPLTTV | 1079 |
| ERNRKRSRSRSSSSGDRKKRRTRSRSPERRHRSSSGSSHS | 1080 |
| SGKRRFLLCLLLFTVITYFFVVIGIAPIFILYELDSPLCW | 1081 |
| LQTRADYLIKLLSRDLAKKEALSGAGSSKRRKARAKKNKA | 1082 |
| SSEIKVKVEPADSVESSPPSITHSPQNELKGTNHSNEKKN | 1083 |
| VSPLPCCTQGHDCQHFYPPSDFTVSTQVFRDMKRSHSLQK | 1084 |
| HVSQAQQETYLGFWINSKKSQCNIFLSGTY | 1085 |
| IWGTDVLKNRSVTGVATKKKKDAVPKPPLSPHKLSIVREC | 1086 |
| IRQHPGHAHYHLPAAYLLGPSRSAVARPPRPGPFLPSQEP | 1087 |
| KHMSLSYVANQEPGILQQKNAVQIISSALDTDNESTKDTE | 1088 |
| QIMLDMLTENLFFDTGMGKSKFLQDMHTLLLTRHRDEHEG | 1089 |
| KVTLLQLLLGHKNEENVEKNTSPQGVHNDVSKFNTQNYAR | 1090 |
| SFTQLSEEIQMAVVWCRSKKLKAQAIFLGNKLLKSNRLKH | 1091 |
| PPRQPFLPGPGQPFLPTHTQPNLQGPLHPPLPPPHQPQPQ | 1092 |
| TISLVAVSDVNKHADRIAFWDDVYGFKMSCMKKAVIPEAV | 1093 |
| LKTVGKLTATQVAKISFFFCFVWFLANLSYQEALSDTQVA | 1094 |
| YLRKDEGSNKQVYSVPHFFLAGAAKERSQMNSQTEDHALA | 1095 |
| GESVLRSVSPVQDLDDDTPPSPAHSDMPYDARQNPNCKPP | 1096 |
| VIQSQSNSFHAKRAEQLKKSILKQAADLTQELPSVLLLHQ | 1097 |
| VSNDLKYDAERDLRDIGAKNILVHSLNKFKYGKISSKHNG | 1098 |
| ARSSRVIKTPRRFMDEDPPKPPKVEVSPVLRPPITTSPPV | 1099 |
| PRSFLAKKLQLVRRVGAPPRRMASPPAPSPAPPAISPIIK | 1100 |
| NGHIHFDNVSVVSLQDGKKEPSSCTCLKGPKLSEIGTIAW | 1101 |
| WAIKLATNAAVTVLRVDQIIMAKPAGGPKPPSGKKDWDDD | 1102 |
| ARCPSARGSGDGEMGKPRNVALITGITGQDGSYLAEFLLE | 1103 |
| KGGWQQKSKGPKKTAKSKKKKPLKKKPTPVLLPQSKQQKQ | 1104 |
| FLLGRWCYQVSHLSWLEKKTATALLESPLSATVEDALQSF | 1105 |
| DSLKNVIARAISKLPIVHFCSSKPRVEYSTKIVEVFCGKE | 1106 |
| LPTMPPPVLPPSLPPPVMPPALPATVPPPGMPPPVMPPSL | 1107 |
| DSEVADSPSSDERRIIETPPHRY | 1108 |
| ILDCNSVRQSIMSVCFFFFLLYSQHDV | 1109 |
| IILDCNSVRQSIMSVCFFFFLLYSQHDV | 1110 |
| HERRVFHLTVAEPHAEPPPRGSPGNGSSHSGAPGPDPTLA | 1111 |
| DLEPHSFGGLLEGIRGASGGAGGRSLDSRLELASLGLGAP | 1112 |
| GAGQHPQPQPPLHKANQPPHGVPQLSLYEHFNSPHPTPAP | 1113 |
| DISQDNALRDEMRALAGNPKATPPQIVNGDQYCGDYELFV | 1114 |
| KSGRYMELEQRYMDLAENARFEREQLLGVQQHLSNTLKMA | 1115 |
| DLIVRCEAGEGECRTFMPPRVTHPDPTERKWAEAVVRPPG | 1116 |
| EKYSNLVQSVLSSRGVAQTPGSVEEDALLCGPVSKHKLPN | 1117 |
| KAKEAAALGSRGSCSTEVEKETQEKMTILQTYFRQNRDEV | 1118 |
| GGWQQKSKGPKKTAKSKKKKPLKKKPTPVLLPQSKQQKQK | 1119 |
| QIVSASVLQNKFSPPSPLPQAVFSTSSRFWSSPPLLGQQP | 1120 |
| LQRANRTGGLYSCDITARGPCTRIEFDNDADPTSESKEDQ | 1121 |
| VEHHTYHIKNYIMNKDLLRRVLVLMNSKHTFLALCALRFM | 1122 |
| PITTYPPYVPEYSSGLFPPSSLLGGSPTGFGCKSRPKARS | 1123 |
| ITALLGSLNSCCNPWIYMFFSGHLLQDCVQSFPCCQNMKE | 1124 |
| PHHDDINFYSERKQNRPFFFACVPADSLEVIPKTIRWTIP | 1125 |
| ARQDLGPSYNGWQVLDATPQEESEGVFRCGPASVTAIREG | 1126 |
| LPPGPPSIFPDCPRECYCPPDFPSALYCDSRNLRKVPVIP | 1127 |
| WFGKNCSEPYCPLGCSSRGVCVDGQCICDSEYSGDDCSEL | 1128 |
| RRGGDHVALQPLRSEGGPPTPHRSIFAPHALPNRNGSLSY | 1129 |
| WVGRWFRPRKGTLGAMDLGGASTQITFETTSPAEDRASEV | 1130 |
| TADEPMVFVDDQLPCNVTFFNASHVVCQTRDLAPGPHYLS | 1131 |
| CLKKGDFSLYPTSVHYQTPLGYERITTFDSSGNVEEVCRP | 1132 |
| RAGRGGLGPPAGVANSLPPQLFAAVSRGCCTSLTHLDASR | 1133 |
| PDPLGPRSQPACQVAHDPPRACPLCSQGTKTLSGSIAPMD | 1134 |
| GQSFSTDAAGSRGGSDGTPRGSPSPASVSSGRKSPHSKSP | 1135 |
| QGRRGPPGAPGEMGPQGPPGEPGFRGAPGKAGPQGRGGVS | 1136 |
| KORALPSLDIVVWSELPPGAGLGSSAAYSVCLAAALLTVC | 1137 |
| GPPGGAGEGGPPAQAPPPPQQPPTAPPSGLKKYEEPLQSM | 1138 |
| HFIKPLLLSEVLAWEGPFPLSMEILEVPEGRPIFLSPWVG | 1139 |
| YFKKLVLNKAILLQVIAKKDDKYTVNIQSVEASENIDVIS | 1140 |
| ARLQTEACRLGQLHPAAPGGLAKVQEAWATLQAKAQERGQ | 1141 |
| VGGEGAEEQPPGAERTFCLSLPDVELSPSGGNHAEYQVAE | 1142 |
| ASPTGDMAVGSPLMQEVGSPKDPGKSLPPVPPMGLPPPQE | 1143 |
| SEEAATWRGRFGPSLVRGLLAVSLAANALFTSVFLYQSLR | 1144 |
| RILIHGLQGASEPPPPLPPLAGVLPRAAQPR | 1145 |
| RFLQLGAKVPKGALLLGPPGCGKTLLAKAVATEAQVPFLA | 1146 |
| CPQDQSPDRVGTEMEQVSKNEGCQAGAELEELSKKAGPEE | 1147 |
| PTATGVQPESSASIVTSYPPPSYNPTCTAYTAPSYPNYDA | 1148 |
| SNGGTCYDSGDTFRCACPPGWKGSTCAVAKNSSCLPNPCV | 1149 |
| GATVTLRCVGNGSVEWDGPPSPHWTLYSDGSSSILSTNNA | 1150 |
| SEDLAPSLGETWKDESVPQVPAEGVDDTSSSEGSTVDCLD | 1151 |
| IQATNASGSPTSMLVVDAPQCPQAPINSQCVNTSQAVQDP | 1152 |
| RAFSPKFGELVAEEARRKGELRYMHSRVVANSEEIAFYGG | 1153 |
| APAAQTPLLGRFLGVGAPSPAISLRNFGRVRGTPRPPHLL | 1154 |
| NVFEPKPSVPEYKVASVGGSRCLLLHYSVSKAIWDGLILL | 1155 |
| METCRRLIKGSADRNSPSPSSVASSDSGSTDEIQDEFERE | 1156 |
| FFRLIKIKIIVKDTNDNAPMFPSPVINISIPENTLINSRF | 1157 |
| LQPHHLPPPPLPPPPVMPGGGYGDWQPPPPPMPPPPGPAL | 1158 |
| KVRLEGRSTTSLSVSWSIPPPQQSRVWKYEVTYRKKGDSN | 1159 |
| AEEDEDLEGPPSYKPPTPKAKLEAQEMPSQLFTLGASEHS | 1160 |
| GERDTLAGQTVDLQGEVDSLSKERELLQKAREELRQQLEV | 1161 |
| TTDERGPPGEQGPPGPPGPPGVPGIDGIDGDRGPKGPPGP | 1162 |
| RQPPPFPPNPMGPAFNMPPQGPGYPPPGNMNFPSQPFNQP | 1163 |
| CKPERDGAESDASSCDPPPAREPPTSPGAAPSPLRLHRAR | 1164 |
| KHKTTPLPPPRLADVAPTPPKTPARKRGEEGTERMVQALT | 1165 |
| PSSEACGEAQRLPSAPSGGAPIRDMGHPQGSKQLPSTGGH | 1166 |
| TDGSPHCVFWDHSLFQGRGGWSKEGCQAQVASASPTAQCL | 1167 |
| SGGGGTAGARGGGGGTAAPQELNNSRPARQVRRLEFNQAM | 1168 |
| ERQPPALKAYPAASTPAAPSPVGSSSPPLAHEAEAGAAPL | 1169 |
| PKRYKANYCSGQCEYMFMQKYPHTHLVQQANPRGSAGPCC | 1170 |
| GLDELDGVKAACPCPQSSPPEQKEAEPEKRPKKVSQIRIR | 1171 |
| MTSLFRRSSSGSGGGGTAGARGGGGGTAAPQELNN | 1172 |
| PPPPQKRYTAAGAGAGGTPDYDPHAHGLQGNGSYGTPHIQ | 1173 |
| LMNLSAHLNDPQPIEMTVKKTLSNFRRTHHDNWQEHKQQF | 1174 |
| TPANSRTLTRAASLRGGVGAPGSPSTPPTRFFTEKKIPHE | 1175 |
| ELWLRLRGKGLAMLHVTRGVWGSRVRVWPLLPALLGPPRA | 1176 |
| RYIRELQYNHTGTQFFEIKKSRPLTGLMDLAKEMTKEALP | 1177 |
| GSQYGMHPDQRLLPGPSLGLAAAGADDLQGSVEAQCGLVL | 1178 |
| VCSSPDYLREPKYYPGGPPTPRPLLPTRPPASPPDKAFST | 1179 |
| TPQKNGRVQEKVMEHLLKLFGTFGVISSVRILKPGRELPP | 1180 |
| HCSNVCSNDPPCFSVMIPPNDSRARSGARCMFFVRSSPVC | 1181 |
| EVRLRRNASSAGRLQGLAGGAPGQKECRPFEVYLPSGKMR | 1182 |
| NPSIRAITLGHGHILVGTKNGEILEIDKSGPMTLLVQGHM | 1183 |
| NESYQQSCGTYLRVRQPPPRPFLDMGEGTKNRIITAEGII | 1184 |
| ASSLSRPWEKTDKGATYTPQAPKKLTPTEKGRCASLEEIL | 1185 |
| MDDVPAPTPAPAPPAAAAPRVPFHCS | 1186 |
| KLRDSMEQAVLDSMGSGKKGQDVGAPNGALSRLDQLSGAQ | 1187 |
| PLKMNPNILSQDSQHVNLFFDKNDENVILQKTTNESMENS | 1188 |
| NLEPGFISIVKLESPRRAPRPCLSLASKARMAGERGASAV | 1189 |
| GTVYHDMGNINLTFFTTKKKYDRMENLKLIVRALNAVQPQ | 1190 |
| MKITNGRHGDSAGAEGTMENFTALFGAQADPPPP | 1191 |
| LKEQLHQKDQKILLLLEEKEMIFRDMAECSTPLPEDCSPT | 1192 |
| RFQELKAQRESKEALEIEKNSRKPPPYKHIKANKVIGKVQ | 1193 |
| KEYLQEKAKEKYQEWLKKKNAEECERKKKEKEKEKQQQAE | 1194 |
| TAKGGVGKLVTLRNVSTKKIPTVNRITPKTQGTNQIQKNT | 1195 |
| GSVLKNGSLTNHFSFEKKKARVAVLISGTGSNLQALIDST | 1196 |
| KRSQENEWVKSDQVKKRKKKRKDYQPNYFLSIPITNKEII | 1197 |
| PGGLEGRLQATGQARPPAPRPFHHGQYYGYLSSSSPGEVE | 1198 |
| WWLTGSNLTLSVNNSGLFFLCGNGVYKGFPPKWSGRCGLG | 1199 |
| MILSSYFINFIYLAKSTKKTMLTLTLVCAITFLLVCSGTF | 1200 |
| KVRYVVSKASVQTQPAIKKDASAQQDSYEFVSPSPPADVS | 1201 |
| KTFRRSSHLTAHQSIHADKKPYECKECGKAFKMYGYLTQH | 1202 |
| FATTLWGVHSAQTEKEKKKESSNCGRRNVFSYGRVKLCST | 1203 |
| SVFGKEVTLETLVKDLKKKIPSLSFSPLKPNGRISVEGSF | 1204 |
| ETADRELLPTFHHVSVYPKKELPLFIHFTAGFCSSTAMIA | 1205 |
| ASINKKLGLLSYKDRIRKKESEVLCSTTETLEEKNENMKL | 1206 |
| DTVGTLSLIMLAQAQEVFFLKATRDKMKDAIIAKLANQAA | 1207 |
| LDDLITPAKLSVGFELLRKMGWKEGQGVGPRVKRRPRRQK | 1208 |
| PDEVCYRVLMQLCSHYGQPVLSVRVMLEMRQAGIVPNTIT | 1209 |
| TEDIKSAFAPFGKISDARVVKDMATGKSKGYGFVSFYNKL | 1210 |
| SIINTLQTQVEVKKRRHRLKRHNDCFVGSEAVDVIFSHLI | 1211 |
| SDNFNTGNMTVLSPYLNTTVLPSSSSSRGSLDSSRSEKDR | 1212 |
| SLLHTAGGGSHGQLGSGSSSEASSVPHLLAQPSVSLGDQP | 1213 |
| QLHHLKLSEDEETVYNVFFARSRSALQSYLKRHESRGNQS | 1214 |
| LRVIGRGSYAKVLLVRLKKTDRIYAMKVVKKELVNDDEDI | 1215 |
| NQSVEQMCNLLLKDQKPKKQGKYICEYCNRACAKPSVLLK | 1216 |
| VDSGDSEVVDGLMLQHITLLMCSAYRNQLLNIFVRPSLVA | 1217 |
| ELFFLDIHNIHVMRESLKKVKDIVYPNVEESHWLSSLEST | 1218 |
| FDLDGDECLSHEEFLGVLKNRMHRGLWVPQHQSIQEYWKC | 1219 |
| VLRGHEFLSHPFAVTLYGGEVYWTDWRTNTLAKANKWTGH | 1220 |
| KAEDGATPSPSNETPKKKKKRFSFKKSFKLSGFSFKKNKK | 1221 |
| SKAKAEKPPLSASSPQQRPPEPETGESAGTSRAATPLPSL | 1222 |
| SAFNLLMHYPPPSGAGQHPQPQPPLHKANQPPHGVPQLSL | 1223 |
| FHLVPNHIVVSAEGNISKKTECLGRALKFDKVGLVQYQST | 1224 |
| TLTILRLSFCTNMEIPHFFCDPSEVLKLACSDTFINNIVM | 1225 |
| DPLTKPMQYKVVVPKIGNILDLCTALSALSGIPADKMIVT | 1226 |
| KRSQENEWVKSDQVKKRKKKRKDYQPNYFLSIPITNKEII | 1227 |
| LSTRHVHLECRLQLWWCGGAPDSSIPDDHQGEKGQGGTEG | 1228 |
| EARRRAHDQLLDLKSSLLKKADTLIGEIFNSVREELKFKH | 1229 |
| IRTHTGEKPYECNICKVRFTRQDKLKVHMRKHTGEKPYLC | 1230 |
| KGVQSLLMYKDGDSVLQRGGSLRAPALPSRSDRLQQRLPI | 1231 |
| NRGEIKGSESATYVPVAPPTPAWQPEIKPEPAWHDQDETS | 1232 |
| APLTMASPAMLGNVESGGPPPPTASQPASVNIPGSLPSST | 1233 |
| QEFLHRYQELLDDNQAPFFLFTCAMRWLAVRLDLISIALI | 1234 |
| TVDADTVTELAQVIVSRGGRFLEAPVSGNQQLSNDGMLVI | 1235 |
| DPSCTVGFYAGDRKEFETLCSELTRVLSSSSATERYPMFT | 1236 |
| KVQEQLKITNLRVQLLKRQSCPCQRNDLNEEPQHFTHYAI | 1237 |
| NNTWEGHYYHYSDPVCKHPTFSIYARGRYSRGVLSSRVMG | 1238 |
| RWRRRGQPMFLALDRRGGPRPGGRTRRYHLSAHFLPVLVS | 1239 |
| EVPAAASQPTFLPWVPERGGGELDLVVRELQALEEELREA | 1240 |
| GPALEEAAGPLVPGLVLGGFGKRKAPKVQPFLPRWLAEPN | 1241 |
| AARGTWWNRPGGTSGSGEGVALGTTRKFQATGSRPAGEED | 1242 |
| VAAGKAKKQVFYGEEERLKKPPRLQESCDSDHGGGRPAAA | 1243 |
| DEHSSDPYHSGYEMPYAGGGGGPTYGPPQPWGHPDVHIMQ | 1244 |
| LLDTKDQSHDLGLHVLSCRNNPLIIPVVHDLSQPFYHSQAVRV | 1245 |

### XVII. Examples

The following examples are given for the purpose of illustrating various aspects of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred aspects, are exemplary, and are not intended as limitations on the scope of the invention. Changes therein and other uses which are encompassed within the spirit of the invention as defined by the scope of the claims will occur to those skilled in the art.

### Example 1: Identification and Validation of Frameshift Neoantigens for Mismatch-Repair Deficient Lynch Syndrome

Lynch Syndrome (LS) is the cause of ˜2.5% of all diagnosed colorectal cancers (CRC). LS patients are at high-risk for the development CRC, with an estimate lifetime risk of 70-80%. LS patients harbor germline mutations in one of the Mismatch Repair (MMR) system genes (MLH1, MSH2, MSH6, PMS2, or TACSTD1/EPCAM). MMR-deficient (dMMR) manifests into microsatellite instability (MSI) and the generation of frameshift peptides (FSP) which become neoantigens (neoAg). neoAg are presented by MHC class I and II and recognized by adaptive immune system. Immunogenic neoAg likely harbor an effective means for CRC immune-interception strategies such as an immunopreventive vaccine for LS carriers.

The inventors utilized paired whole-exome sequencing and mRNAseq in LS CRC (stage I-III) and pre-cancers to catalog and identify the most frequently recurrent neoAg present in LS patients, and used in-silico metrics such as HLA genotype, mutational frequency, HLA binding affinity, and expression levels to predict immunogenicity. To validate the computational predictions, the inventors harvested cytotoxic lymphocytes from a total of 3 LS patients and generated neoAg-loaded tetramers to mimic MHC-I presentation of 10 different neoAg from the prediction list. After neoAg-specific CTLs were enumerated and isolated using tetramer stains, ELISpots, and a 15-plex cytokine profiling ELISA assay were used to ascertain the immunogenic potential of each neoAg.

MHC-tetramer staining revealed that neoAg-specific CTLs comprised approximately 0.5-1% of total peripheral CTL population, which is consistent with previous studies. ELISpots performed using CTLs showed significant secretion of IFNγ (spot forming units) upon overnight stimulation with neoAg-loaded tetramers compared to controls. A 15-plex cytokine profile using CTLs from one patient identified significant activation of proinflammatory (IL-1a, IL-1b, IL-12, IL-17, IL-23) and proliferative (IL-2, IL-15) cytokines upon neoAg stimulation compared to the unstimulated control.

These results provide strong evidence to suggest the in silico computational pipelines accurately predict the immunogenicity of LS neoAg and that these neoAgs have the potential to mount an immune response consistent with previously published work performed in other cancers. This study provides the foundation for developing an immunoprevention vaccine for LS carriers.

Patients and Specimen collection: All patients for this study had a confirmed diagnosis of LS (n=28). Patient characteristics are shown in the chart below:

| Characteristic | N |
|---|---|
| Age ± SD | 52 ± 14 |
| Gender | |
| Female | 18 |
| Male | 10 |
| Race | |
| Caucasian | 23 |
| Other | 4 |
| Not disclosed | 1 |
| Ethnicity | |
| Not hispanic or latino | 21 |
| Hispanic or latino | 6 |
| Not disclosed | 1 |
| dMMR gene | |
| MLH1 | 9 |
| MSH2 | 10 |
| MSH6 | 6 |
| PMS2 | 2 |
| Not detected | 1 |
| Cancer Status | |
| Previvor | 6 |
| Active Cancer | 8 |
| Survivor | 14 |
| Colorectal Neoplasm* | |
| Inflammatory polyp | 2 |
| Hyperplastic polyp | 3 |
| Tubular adenoma | 24 |
| Tubulovillous adenoma | 1 |
| Sessile serrated adenoma | 3 |
| Adenocarcinoma In Situ | 1 |
| Adenocarcinoma Stage I | 2 |
| Adenocarcinoma Stage II | 3 |
| Adenocarcinoma Stage III | 4 |
| Premalignant lession size (mm) ± SD | 8 ± 17 |

*One patient can have more than one cancer or neoplasm

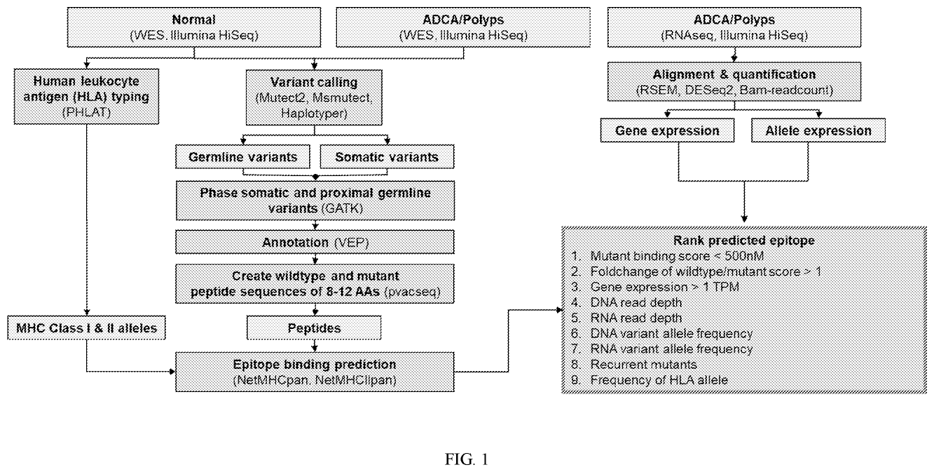

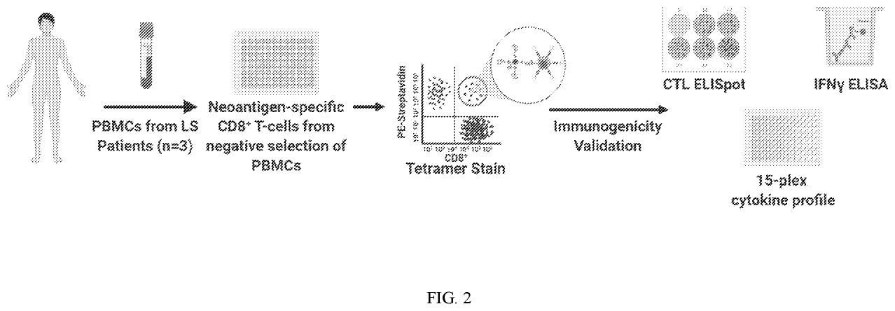

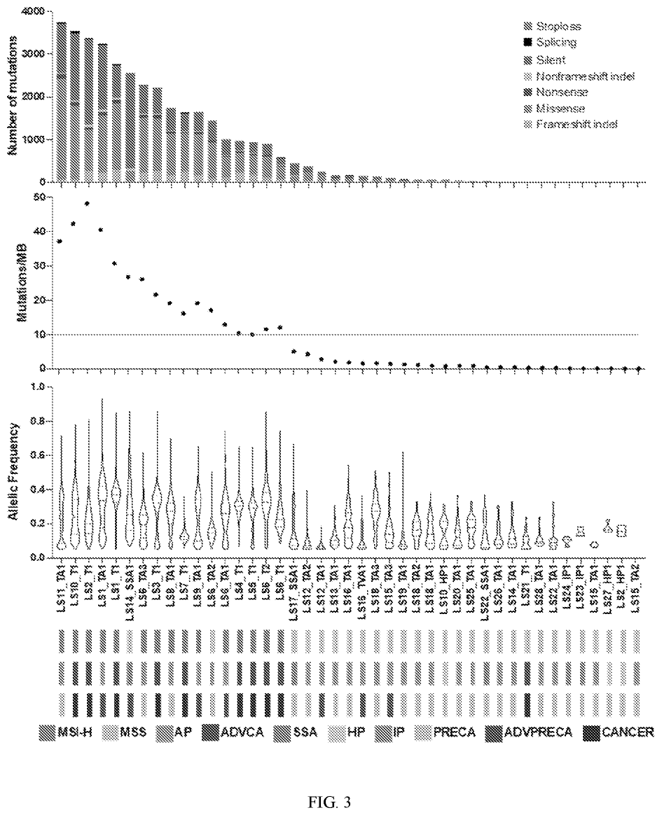

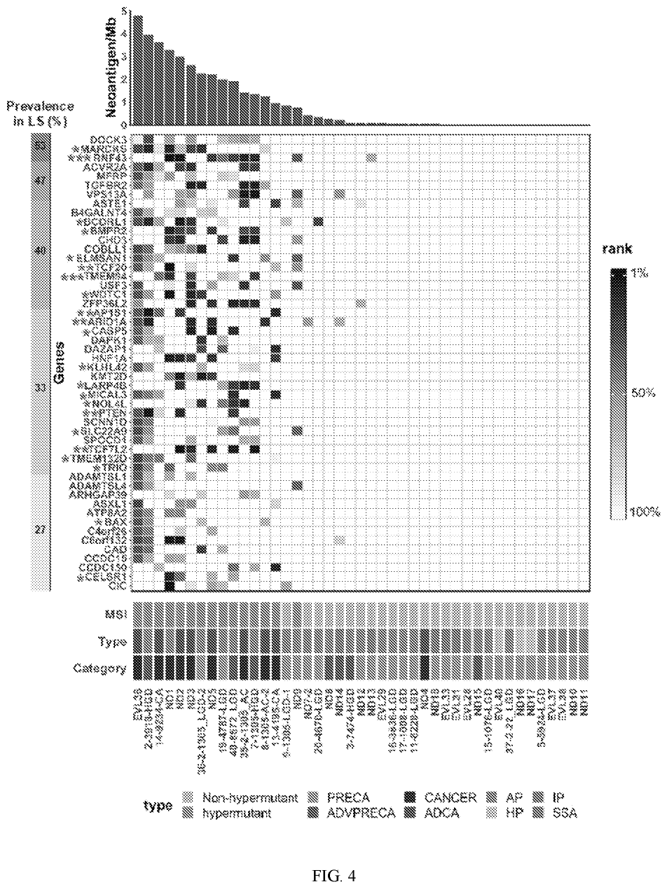

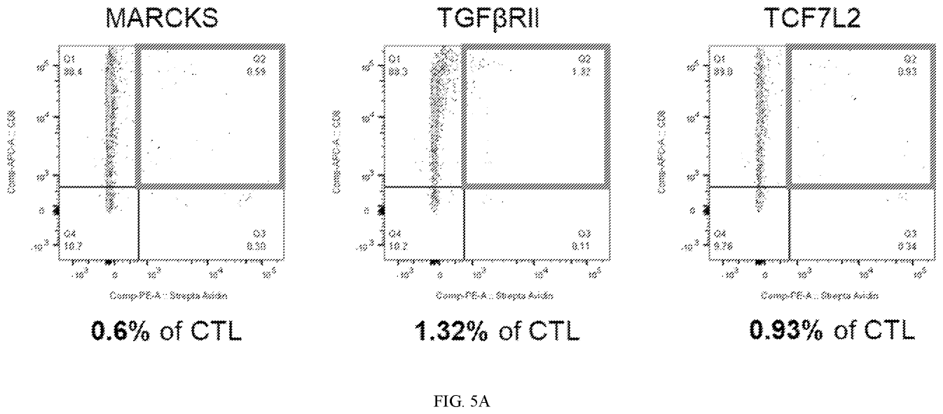

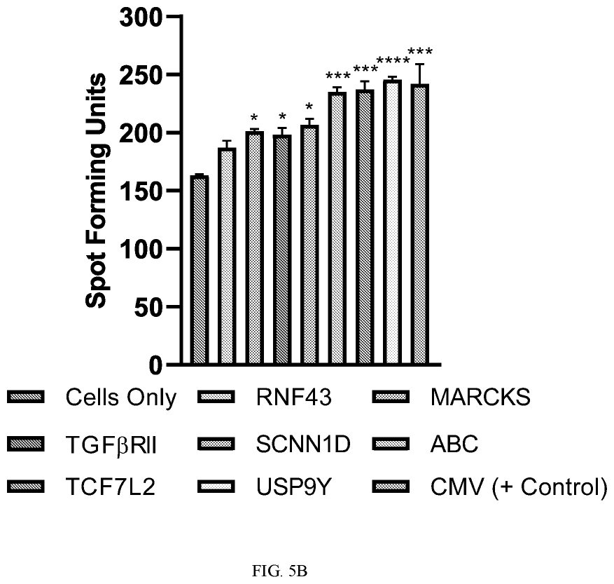

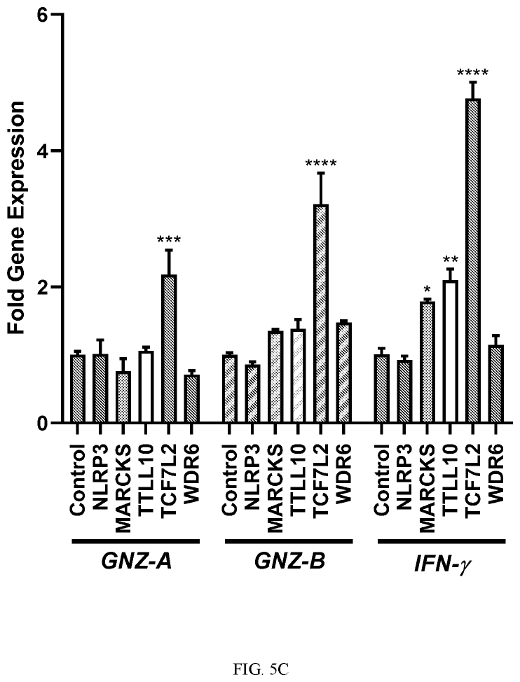

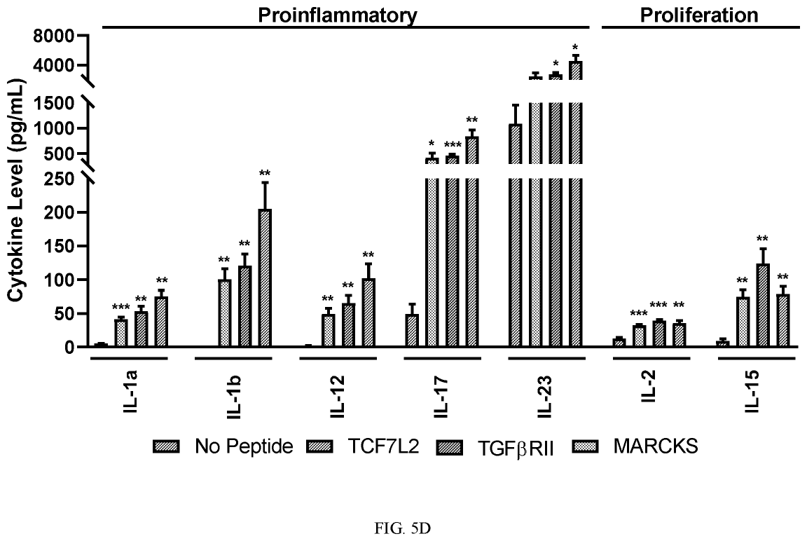

The strategy for in silico neoantigen prediction is shown in FIG. 1, and the in vitro validation pipeline is shown in FIG. 2. FIGS. 3-4 show the mutation frequency and neoantigen sequencing. FIG. 5A-D shows the validation of neoantigen immunogenicity.

In conclusion, the inventors performed paired whole-exome sequencing (WES) and mRNAseq of LS CRC (stage I-III) and precancers from the LS patient cohort. A state-of-the-art bioinformatics pipeline predicted a catalog of recurrent and highly immunogenic neoAg. The inventors validated the immunogenicity of a few peptides using MHC class I tetramers and ELISPOT. The in vitro validation confirms the accuracy of in silico prediction of the immunogenic neoAg. This data supports using these neoAg as a vaccine-based immunoprevention strategy for LS patients to prevent the development of CRC.

### REFERENCES

The following references, to the extent that they provide exemplary procedural or other details supplementary to those set forth herein, are specifically incorporated herein by reference.
1. Lynch H T, Snyder C L, Shaw T G, Heinen C D, and Hitchins M P. Milestones of Lynch syndrome: 1895-2015. Nat Rev Cancer. 2015; 15(3):181-94.
2. Bonadona V, Bonaiti B, Olschwang S, Grandjouan S, Huiart L, Longy M, et al. Cancer risks associated with germline mutations in MLH1, MSH2, and MSH6 genes in Lynch syndrome. JAMA. 2011; 305(22):2304-10.
3. Ott P A, Hu Z, Keskin D B, Shukla S A, Sun J, Bozym D J, et al. An immunogenic personal neoantigen vaccine for patients with melanoma. Nature. 2017; 547(7662):217-21.
4. Cohen C J, Gartner J J, Horovitz-Fried M, Shamalov K, Trebska-McGowan K, Bliskovsky V V, et al. Isolation of neoantigen-specific T cells from tumor and peripheral lymphocytes. J Clin Invest. 2015; 125(10):3981-91.
5. Wells D K, van Buuren M M, Dang K K, Hubbard-Lucey V M, Sheehan K C F, Campbell K M, et al. Key Parameters of Tumor Epitope Immunogenicity Revealed Through a Consortium Approach Improve Neoantigen Prediction. Cell. 2020; 183(3):818-34 e13.

### Example 2: Identification and Validation of Frameshift Neoantigens for Mismatch-Repair Deficient Lynch Syndrome

Lynch syndrome (LS) patients constitute a well-defined population that will likely benefit from cancer immune-interception strategies given that they develop DNA mismatch repair deficient tumors that generate high loads of neoantigens. The inventors performed in-silico prediction, immunogenicity ranking, and in-vitro validation of highly immunogenic and recurrent frameshift neoantigens (FS-neoAgs) from colorectal cancers (CRC) (n=13) and pre-cancers (n=61) of the LS patient cohort (N=46), using paired whole-exome sequencing and mRNAseq. The inventors showed that mutation burden derived from microsatellite instability is positively correlated with high FS-neoAgs load even in pre-cancers. After testing 154 predicted FS-neoAgs, they demonstrated an in-vitro validation rate of up to 50% in MHC-I restricted FS-neoAgs, when high predicted-immunogenicity and recurrency of the FS-neoAgs within the cohort are considered as factors for their selection. Overall, the mutational data, gene expression data, and FS-neoAgs catalog improve the understanding of LS-derived cancer, which will guide the future development of immunoprevention vaccine strategies.

This study provides the largest LS somatic mutation, gene expression, and FS-neoAgs landscape report presently available with supported evidence of a computational pipeline that accurately predicts the immunogenicity of tumor-derived FS-neoAgs. This computational platform affords the future development and discovery of a universal LS cancer-vaccine.

### A. Introduction

Lynch Syndrome (LS), the leading cause of hereditary colorectal cancer (CRC), represents 2-4% of total CRC and affects more than 1 million carriers in the United States (1). LS arises from heterozygous germline mutations in the DNA mismatch repair (MMR) genes, with MLH1 and MSH2 responsible for more than 70% of LS cases. LS patients have an increased lifetime risk for CRC development that reaches 60% in MLH1 and MSH2 carriers (2). Normal colorectal cells become MMR deficient (dMMR) upon the acquisition of a second somatic hit in the alternate allele of the MMR gene that harbors the germline mutation. This second hit manifests into the accumulation of base-to-base mismatches and insertion-deletion mutations (indels) in microsatellite sequences, which generate neoantigens (neoAg). These tumor-specific antigens are processed and presented, as short peptides loaded onto major histocompatibility complexes (MHC I/II), to T cell receptors (TCRs) on cytotoxic CD8+ T cells, which promotes interferon γ (IFNγ) secretion to kill neoAg producing cancer cells (2). However, when cancer cells become capable of immune evasion, namely through upregulation of immune checkpoint molecules, tumors return to an uncontrolled growth state. Thus, activating CD8+ and CD4+ T cells (helper cells) that recognize neoantigens is important for adaptive immunity against tumors.

Extensive computational algorithms have used next-generation sequencing (NGS) data to rapidly screen the mutational landscape of human cancers, including melanoma and colon (4-7). Such studies have identified a variety of neoantigens that may be recognized by the host's immune system, providing promising avenues for more personalized and focused approaches to activate anti-tumor immunity (8). Given the propensity for an increased mutational burden in dMMR cancers, putative neoAgs characterized from genomic and transcriptomic data in LS patients may provide a similar opportunity to develop novel immunoprevention therapies such as neoantigen-peptide vaccines.

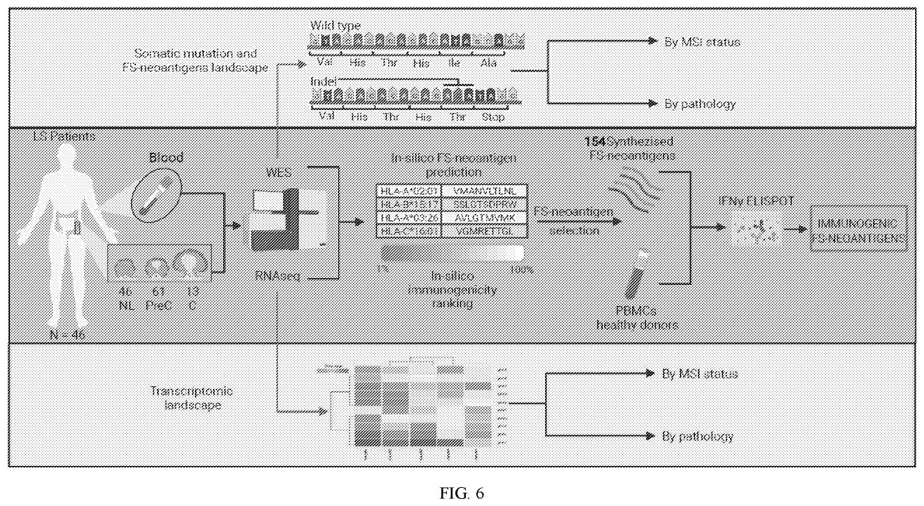

In this study, the inventors have acquired genomic data with paired whole-exome sequencing (WES) and mRNAseq in LS CRC (stage I-III) and precancers (advanced adenomas and adenomas) to catalog and identify the most immunogenic and recurrent frameshift-neoAg (FS-neoags) present in LS colorectal precancers and tumors using innovative bioinformatics. The established pipeline accurately identifies somatic microsatellite (MS) indels by estimating and reducing read-length associated sequencing errors, PCR amplification errors, and other sources of noise. It also accounts for the frequency of a given peptide within the studied cohort, its binding affinity, expression levels, and the individual's HLA genotype. Finally, the inventors employed immunological assays to validate the predicted immunogenicity of FS-neoAgs from the computational methods, thus moving the field closer towards improved immunoprevention therapies for LS cancers. A summary of the present study is illustrated in FIG. 6.

### B. Results

### 1. Demographics and Characteristics of LS Patient Cohort

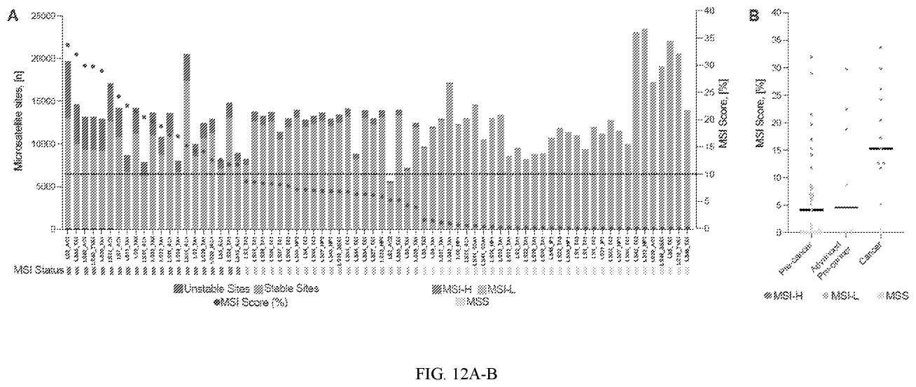

The inventors analyzed a total of 74 colorectal adenomas (polyps) or tumor samples from the lower gastrointestinal tract of 46 LS patients, with matched normal mucosa and peripheral blood. The patient demographics and clinical characteristics are summarized in Table 1, and the pathological features of each polyp or tumor are found in Supplementary Table 1. The mean age of the patient cohort was 52 years (range, 20-80). The majority of patients had a germline pathogenic mutation in MSH2 (N=14) and MSH6 (N=14), followed by MLH1 (N 11), PMS2 (N=5). Two patients met the Amsterdam Criteria but had no germline mutation detected. Among the precancerous lesions, 44 were confirmed as early tubular adenomas, nine as hyperplastic polyps (HPs), two as inflammatory polyps (IPs), two as tubulovillous adenomas, and four as sessile serrated adenomas (SSAs). All cancerous lesions were confirmed as adenocarcinomas (n=13) at different stages. The MSI status of the samples was determined based on the MSI sensor score. Among the 74 samples, the scores ranged from 0% to 33.67%, with 21 samples classified as MSI-H, 22 as MSI-L, and the remaining as MSS (FIG. 12A). The median MSIscore for precancers was 4.1%, with almost half displaying MSI-H (n=9) and MSI-L (n=19). Most cancers (n=10) were MSI-H, with a median MSIscore of 15.3%, as expected (FIG. 12B).

2. Germline Mutation and Second Somatic Hit Analysis of MMR Genes in LS patient Cohort

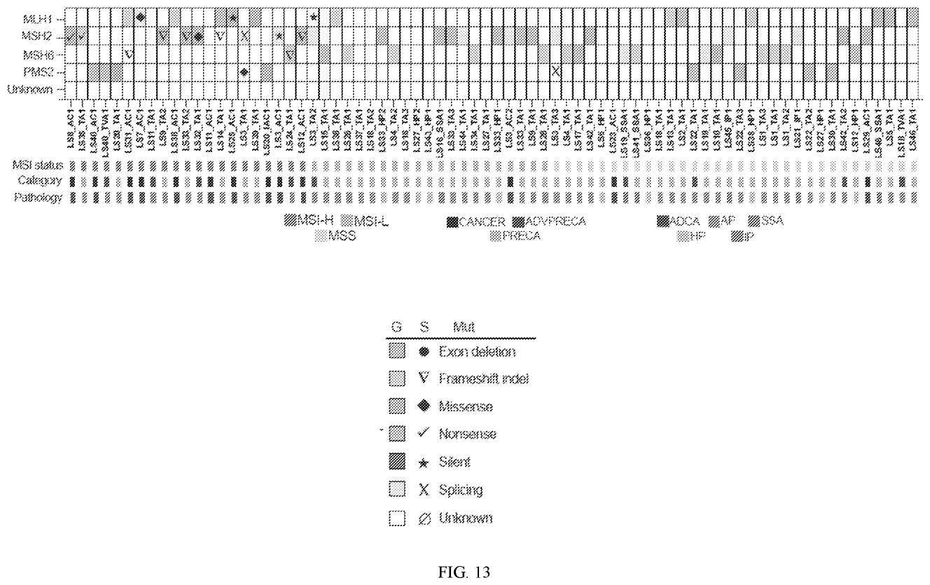

Identification of germline mutations was performed using HaplotypeCaller, as described in the Methods. The types of germline mutations within the MMR genes of the 46 patients consisted of splicing events (13%), frameshift indels (24%), nonsense mutations (26%), exon deletions (11%), missense mutations (9%), and unknown type (17%) for the remaining patients. These germline mutations, together with the second somatic hits that the inventors were able to detect, are shown in FIG. 13. As expected, more than 60% (13/21) of the MSI-H samples had a detectable second somatic hit. The remaining MSI-H samples with an undetected second somatic hit, as well as those patients with an unknown type of germline mutation, could potentially be explained by the lack of sensitivity that WES has when it comes to detecting structural variations, intronic variants and variants that sit in distal regulatory elements of the genome.

3. LS Patients with MSI-H Harbor Somatic Mutation Variations in CRC Associated Genes

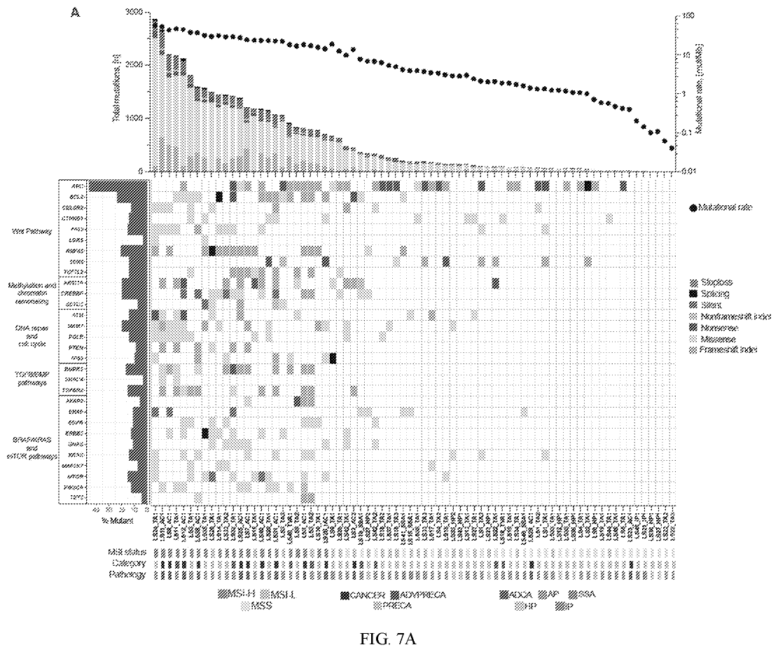

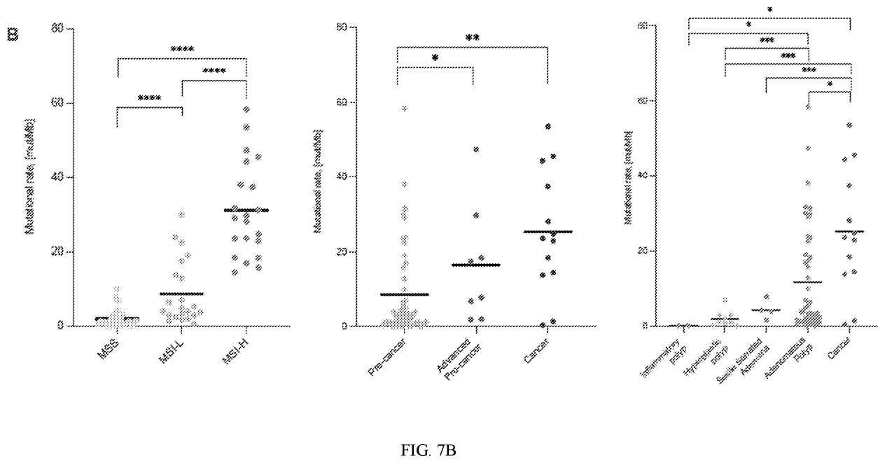

A landscape of somatic mutations in the LS cohort was determined using WES data from the 74 lesion-normal pairs, by combining Mutect2 and MSmutect outputs. The inventors observed a range of (2-2862) mutations per sample, and most of these mutations were missense and frameshift indels (FIG. 7A). Additionally, the inventors detected recurrent deleterious mutations (frameshift indel, nonsense, and stop loss) in several genes of canonical CRC-associated pathways, including WNT, chromatin remodelers, DNA repair, and TGFβ/BMP. For example, within the Wnt pathway genes, APC mutations were identified in 33/74 samples, of which 27 were adenomas (with 15% MSI-H), five were adenocarcinomas (with 100% MSI-H), and one was a HP (no MSI-H). Additionally, BCL9 was mutated in 17/74 samples, of which eight were adenomas (with 75% MSI-H), seven were adenocarcinomas (with 86% MSI-H), and two were SSAs (no MSI-H). Furthermore, this analysis determined that mutations in CRC-associated genes are widely present within the 74 samples, including 14 samples with ARID1A mutations, 11 with TGFBR2, 10 with ATM, CTNNB1, KRAS, SOX9, and TCF7L2, nine with PIK3CA, seven with TP53, 6 with PTEN, and four samples with BRAF mutations. BRAF mutations were only detected in the four SSAs of this sample cohort, in concordance with previous studies (10). Overall, when considering MSI status, tissue category or pathology, the mutational burden was significantly increased in the most advanced level of each of these characteristics (FIG. 7B). MSI-H samples displayed the highest mutational rate when considering the MSI status, cancers had significantly higher mutational rate when looking at the tissue category, and adenocarcinomas, followed by adenomatous polyps, showed the highest level of mutational burden in terms of pathology.

4. In silico Neoantigen Prediction with Immunogenicity Ranking

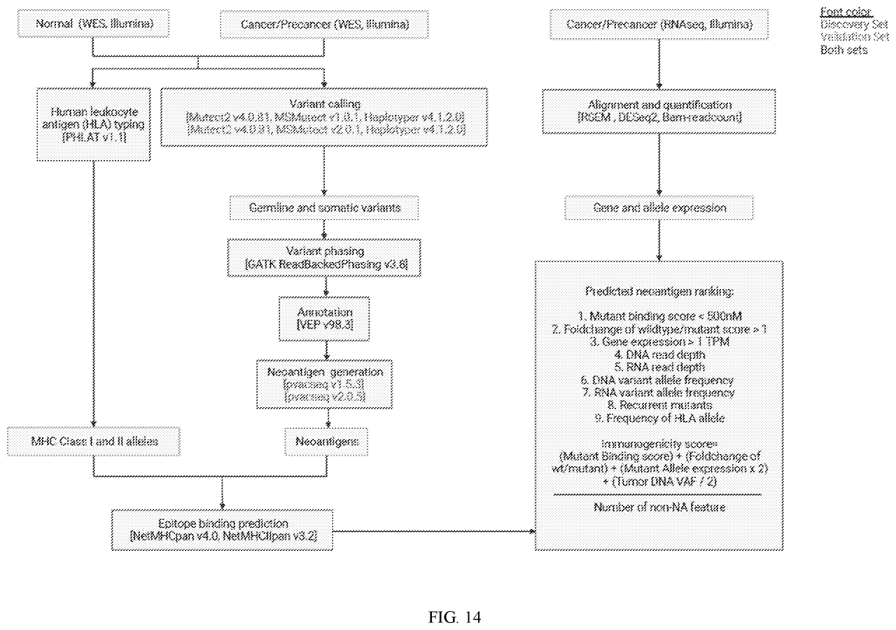

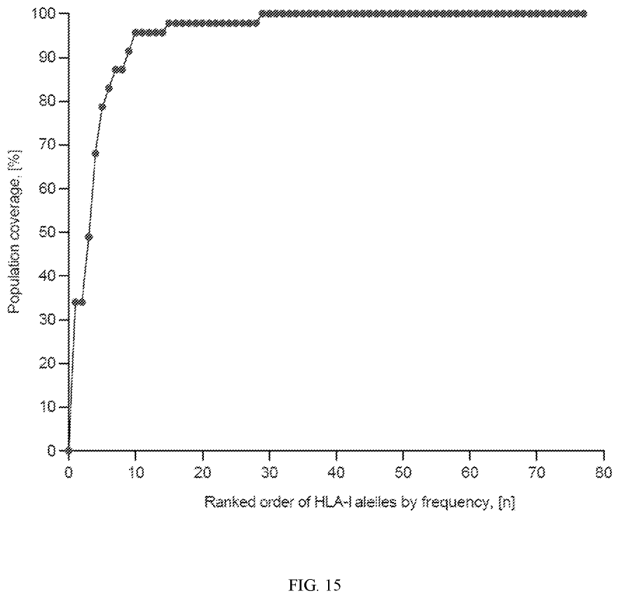

Applying a series of computational methods and bioinformatic approaches, the inventors developed a neoAg prediction pipeline, as summarized (FIG. 14), to identify and catalogue neoAg produced from the frameshift mutations in the LS sample cohort. To do this, the inventors first performed MHC class I and II typing from WES data using PHLAT (11), and determined the ranking of frequencies of the HLA alleles within the LS cohort in this study (Supplementary Table 2). The typing results indicated that the topmost frequent HLA class I alleles were HLA-A*02:01, HLA-B*07:02, and HLA-C*07:02 for each locus, covering 32%, 34%, and 34% of the cohort, respectively. For the HLA class II alleles, the topmost frequent were HLA-DQA1*01:02, HLA-DQB1*06:02, and HLA-DRB1*15:01 for each locus, covering 49%, 36% and 34% of the patient cohort, respectively. Furthermore, more than 90% of the patient population in the cohort contained the top 10 most frequent HLA class I alleles (FIG. 15).

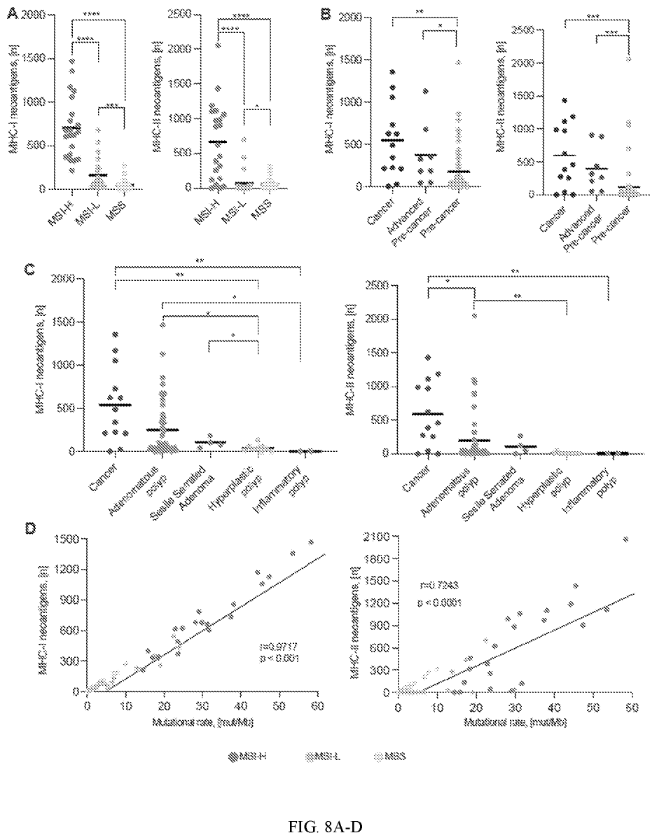

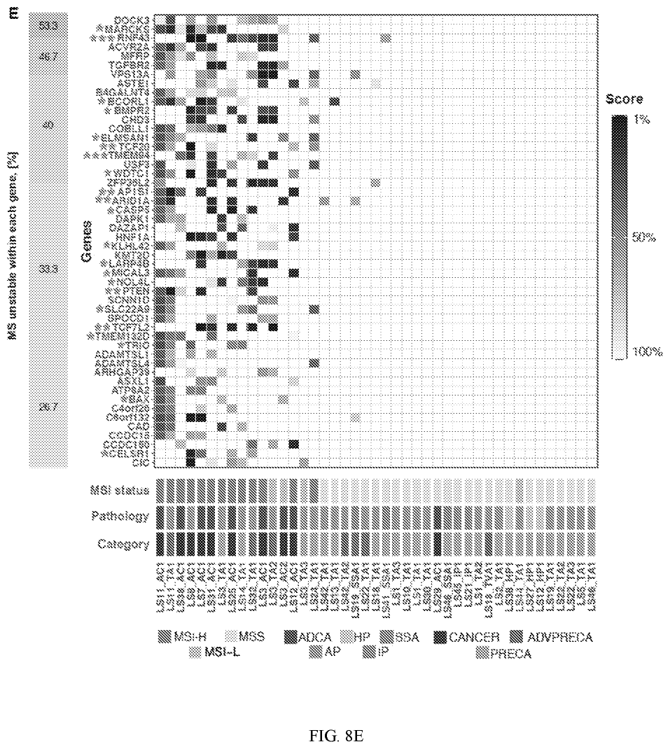

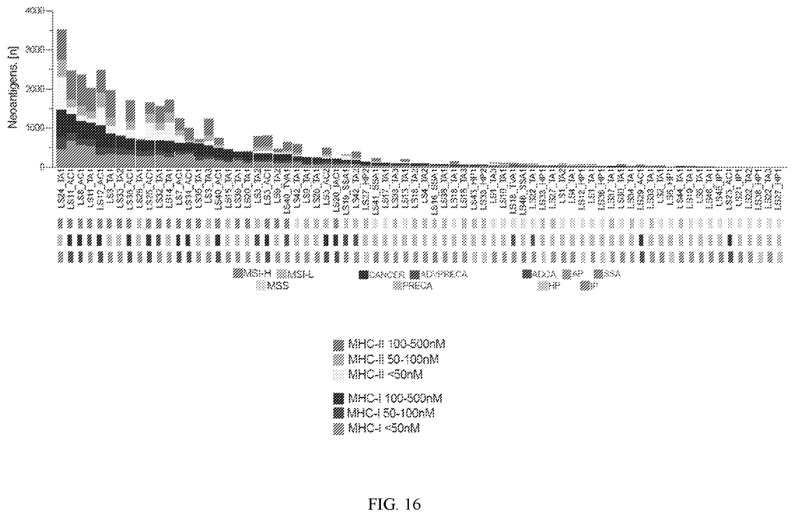

Immunogenicity prediction of potential neoAgs restricted to HLA class I and II epitopes was calculated using the NetMHCpan algorithm, as described in the Methods section. The total number of MHC-I and MHC-II neoantigens predicted per sample ranged from 0 to ˜3500 with a majority of neoAgs bearing a high predicted binding affinity (<50 nM) (FIG. 16). In concordance with the mutational rate, considering MSI status, tissue category and pathology, the number of neoAgs was significantly increased in the most advanced level of each of these characteristics (FIGS. 8A, B and C). MSI-H samples showed significantly higher numbers of MHC-I and MHC-II neoAgs (Mann-Whitney test, P<0.0001; FIG. 8A), as well as cancers, when compared among tissue category or pathology (Mann-Whitney test, p<0.01; FIGS. 8B and C). This was further proved by comparing the number of neoAgs identified per sample with the mutational rate of each sample, which showed a significant positive correlation for both MHC-I and MHC-II neoAgs (Pearson, P-value=<0.001; FIG. 8D).

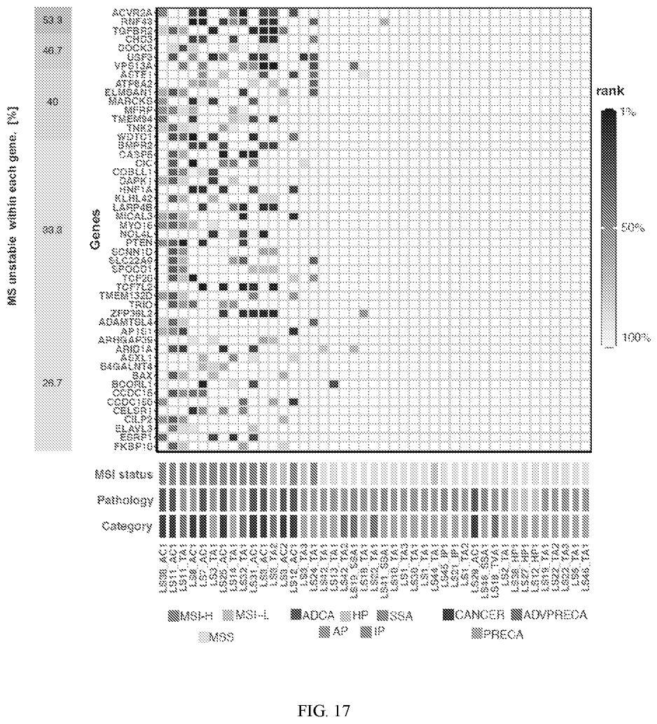

Since the goal of this study is to discover a set of neoAgs with potential to be used as LS cancer-vaccine, and every discovery needs its independent corroboration. the inventors divided the sample cohort into a discovery and a validation set (Supplemental Table 3). Prediction of frameshift indel-derived MHC-I and MHC-II neoAgs was separately performed for each set. MHC-I neoAgs from the discovery set were ranked based on their immunogenicity score (Supplementary Table 4), obtained from the formula described in FIG. 14 and the Methods. Separately. these MHC-I predicted neoAgs were also ranked based on their recurrency within the sample cohort (Supplementary Table 5). FIG. 8E shows the landscape of the top 50 genes that generate the most recurrent MHC-I restricted neoAgs among the discovery sample cohort with their calculated immunogenic score (blue scale). The pipeline identified a set of novel recurrent MHC-I restricted neoAg with high immunogenic potential. predicted to be generated in genes that include RNF43, ACVR2A, BCORL1, BMPR2, and TCF20. Of note. the predicted neoAgs produced from mutated MARCKS, TGFBR2. TCF7L2. and ASTE1 proteins were previously reported in LS patients (11).the inventors also predicted and cataloged the MHC-II restricted neoAgs based on their immunogenicity score (Supplementary Table 6) and recurrence (Supplementary Table 7). The landscape of the top 50 genes generating the most recurrent MHC-II restricted neoAgs among the discovery sample cohort is shown in FIG. 17. Several genes generated recurrent and potentially immunogenic neoAg, restricted to both MHC-I and MHC-II molecules.

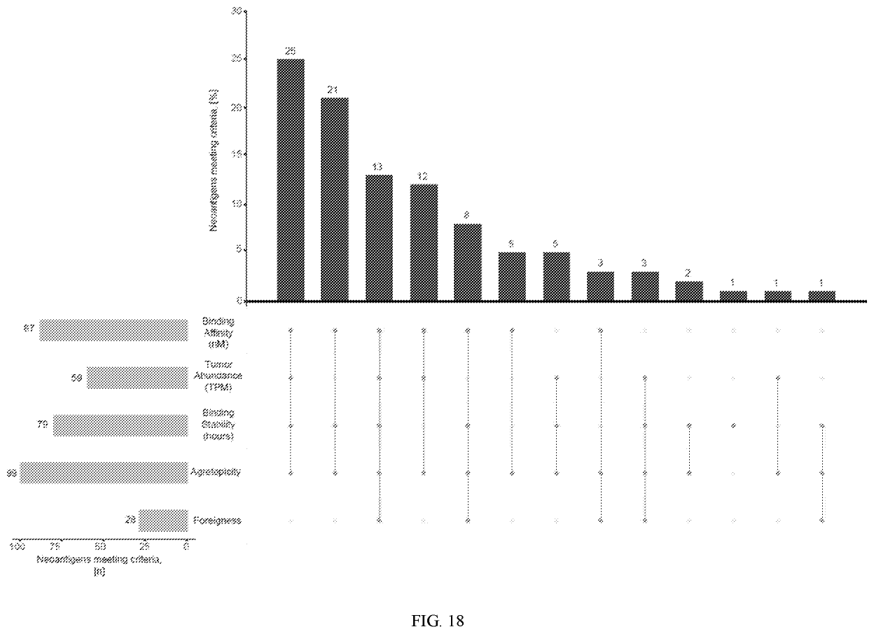

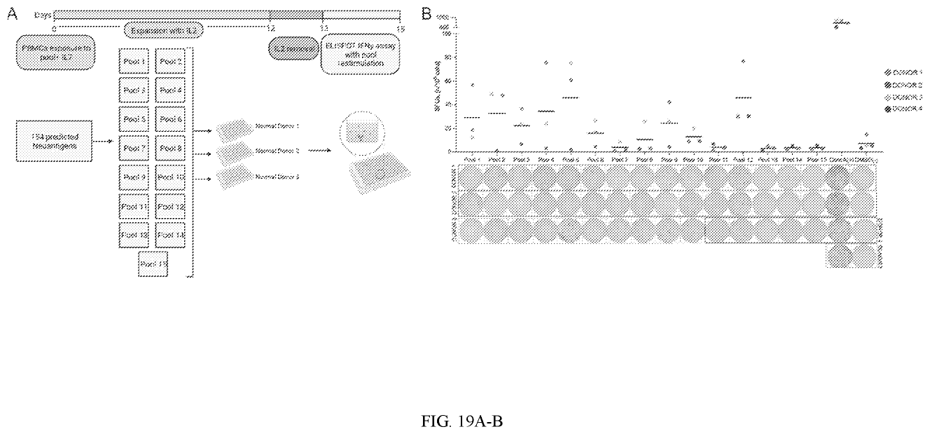

To in-silico validate the performance of the neoAg prediction pipeline, the inventors first benchmarked the MHC-I restricted neoantigen predictions from the discovery set to those of the Tumor Neoantigen Selection Alliance (TESLA). The TESLA platform assessed the level of agreement of 25 different pipelines and ranking systems using a common data set of three melanoma and three non-small cell lung cancers followed by an in vitro validation platform using MHC-I multimer-based assays (12). Their work proposed five robust immunogenicity criteria to rank the potential of predicted neoAg for the presentation and recognition of the immune system: 1. binding affinity <34 nM; 2. tumor abundance >33 TPM (transcript per million); 3. binding stability >1.4 h; agretopicity <0.1 and foreignness >10⁻¹⁶. From the top 100 most immunogenic MHC-I indel-derived predicted neoAg. 25% met all three presentation criteria, which includes binding affinity. tumor abundance, and binding stability, whereas 13% met all five criteria, including the three presentation criteria. plus the recognition criteria (agretopicity and foreignness) (FIG. 18).

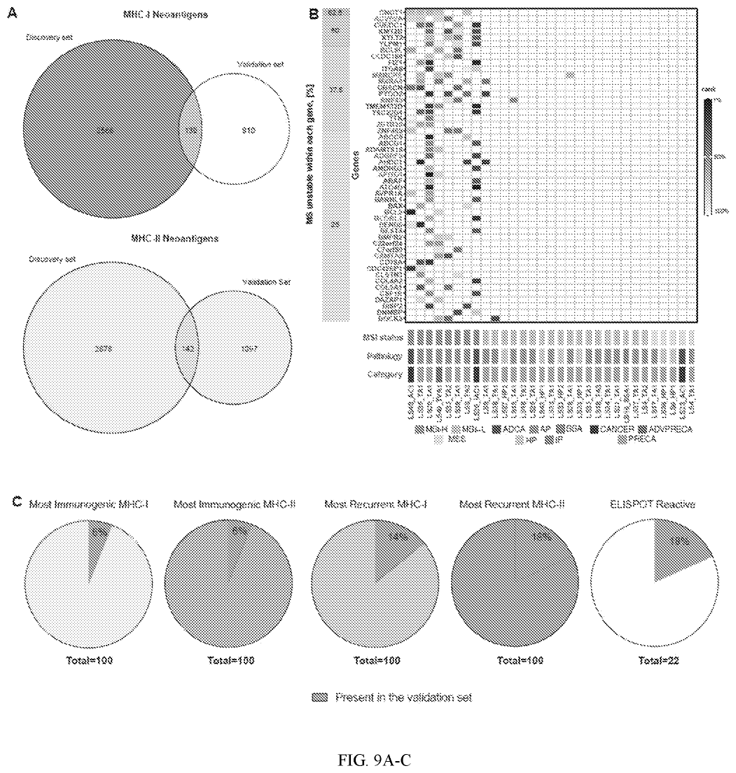

To further validate the in-silico prediction conducted on the discovery set (n=43). the inventors leveraged the validation set of samples (n=31) (Supplementary Table 4) to perform neoAg prediction and assess the level of agreement between the two data sets with respect to shared neoAgs. For MHC-I neoantigens. the inventors found that 130 shared neoAgs between the discovery and validation sets. Conversely. for MHC-II neoantigens, the inventors found 142 shared neoantigens between the discovery and validation sets (FIG. 9A). Notably. among the top 50 predicted neoAg from the validation set. 10 were also present in the discovery set, including CNOT1, ACVR2A, MARCKS, MXRA8, RNF43, BCORL1, and, CAMTA2 (FIG. 9B, light grey font). Of the 100 most immunogenic MHC-I and -II neoAgs in the discovery set. 6% were also found in the validation set. For most recurrent MHC-I and -II neoAgs. 14% and 18%, respectively. were also present in the validation set (FIG. 9C). The inventors cataloged the predicted MHC-I restricted neoAgs from the validation set based on their immunogenicity score (Supplementary Table 8) and recurrence (Supplementary Table 9). Ten percent of the top 100 most immunogenic MHC-I restricted neoags predicted in the validation set were also present in the discovery set. while 18% of the top 100 most recurrent MHC-I neoags from the validation set. were also present in the discovery set. Lists of MHC-II restricted neoAgs based on immunogenicity and recurrence in the validation set are included in Supplementary Tables 10 and 11. respectively. From the validation set neoags prediction. 17% of the top 100 most immunogenic. and 18% of the top 100 most recurrent were also present in the discovery set.

5. Selection of the Predicted neoAg for In Vitro Validation of Immunogenicity in Human Donors

To validate the immunogenicity of the predicted neoAg in-silico, the inventors selected a total of 154 neoAgs from the discovery set to test the immunogenic response of the pooled and individual peptides in ELISpot assays using PBMCs from healthy donors. These MHC-I peptides were selected as follows: 10 were randomly selected from the top 100 most immunogenic predicted neoAgs. 55 from the top 100 most recurrent. 14 which were part of both the top 100 most immunogenic and the top 100 most recurrent MHC-I neoags (Supplementary tables 4 and 5; column: Tested with ELISPOT), and 31 that were not part of either group and had low immunogenicity and no recurrency (Supplementary Table 12). For MHC-II peptides. 20 were randomly selected from the top 100 most immunogenic MHC-II predicted neoAgs. 17 were randomly selected from the top 100 most recurrent MHC-II neoAgs. and 7 were part of both groups. the most immunogenic and most recurrent MHC-II neoAgs (Supplementary tables 6 and 7; column: Tested with ELISPOT).

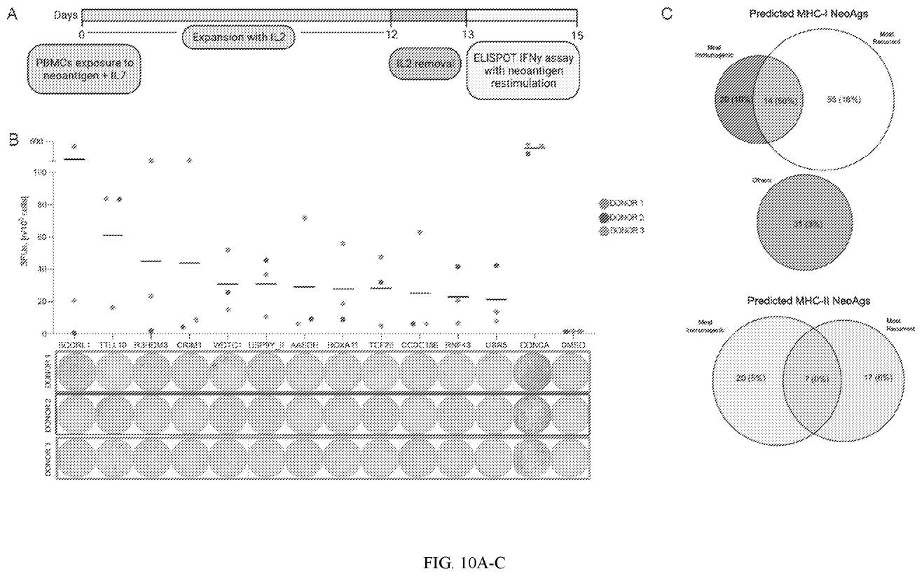

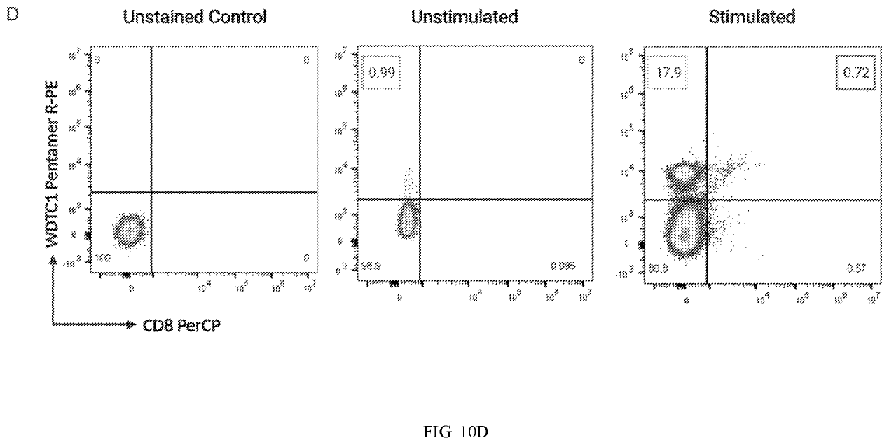

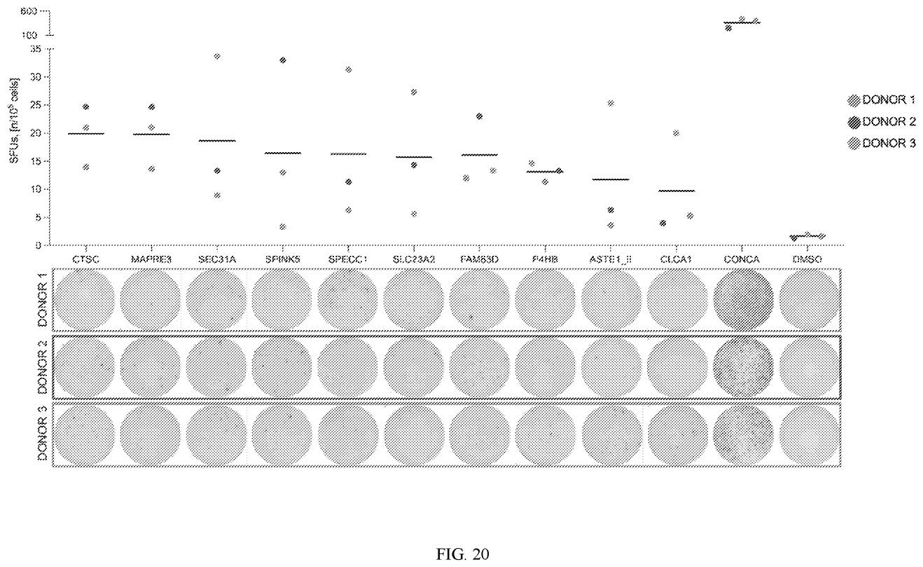

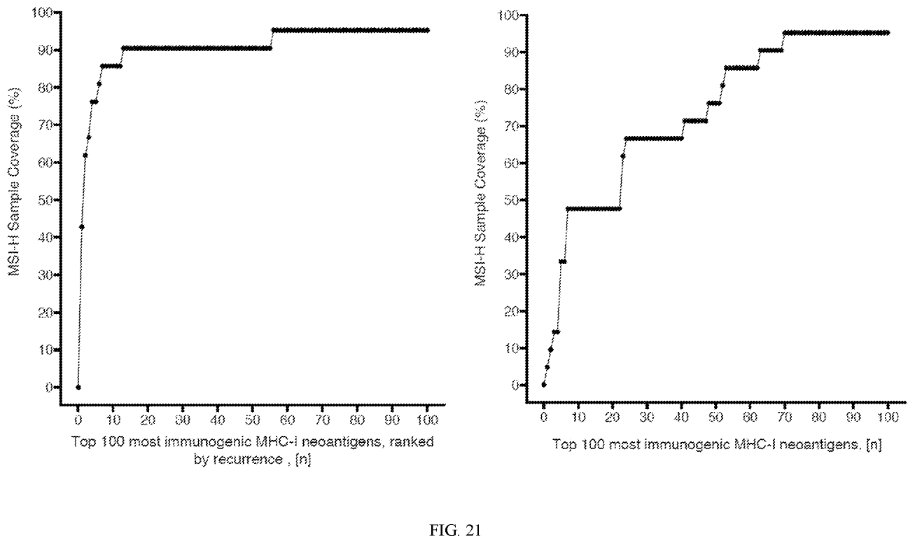

Healthy donor PBMCs were stimulated with 15 peptide pools (Supplementary Table 13) to expand the neoAg-specific CD8⁺ T-cells followed by quantitative ELISpot assays (FIG. 19A) to measure immunogenicity. These result showed that peptide pools 1, 2, 3, 4, 5, 8, 9, and 12 elicited high secretion of IFNγ in one or more healthy donors PBMCs when compared to DMSO control cells (FIG. 19B). To ascertain the immunogenicity of individual peptides within each immunogenic pool. a deconvolution protocol (see Methods) was implemented (FIG. 10A). This data showed that a total of 20 MHC-I and 2 MHC-II predicted neoAgs (from 8 immunogenic pools) elicited significant secretion of IFNγ in healthy donor PBMCs. compared to unexposed control cells (Supplementary Table 14). The top 12 most reactive (immunogenic) frameshift antigenic peptides were generated from the following genes: BCORL1, TTLL10, R3HDM3, CRIM1, WDTC1, USP9Y, AASDH, HOXA11, TCF20, CCDC186, RNF43, and UBR5 (FIG. 10B) and the other 10 reactive peptides are shown in FIG. 20. ELISpot data showed that at least 10% of the most immunogenic. 16% of the most recurrent, and 50% of the peptides that were part of both the most immunogenic and most recurrent MHC-I neoAgs showed in vitro reactivity. which validates the in-silico neoAg prediction pipeline (FIG. 10C). Three percent of MHC-I neoAgs not predicted to be highly immunogenic or recurrent (Others). 5% of the top 100 most immunogenic MHC-II predicted neoags, and 6% of the top 100 most recurrent MHC-II predicted neoags elicited an immunogenic response as assessed by ELISpot assays. Additionally. 18% of all of these elispot-reactive neoags were also predicted from the validation set (FIG. 9C). Based on these results. the highest percentage of in-vitro validation was achieved with the MHC-I predicted neoags that were part of both the most immunogenic and the most recurrent neoags. These two factors, when combined. also showed the best population coverage in-silico with the top 10 most immunogenic plus recurrent MHC-I neoags being present in more than 85% of the cohort. while the top 10 most immunogenic (only) covered less than 50% (FIG. 21).

### 6. Validation of Predicted Neoantigens Immunogenicity in the LS Model of Rhesus Macaques

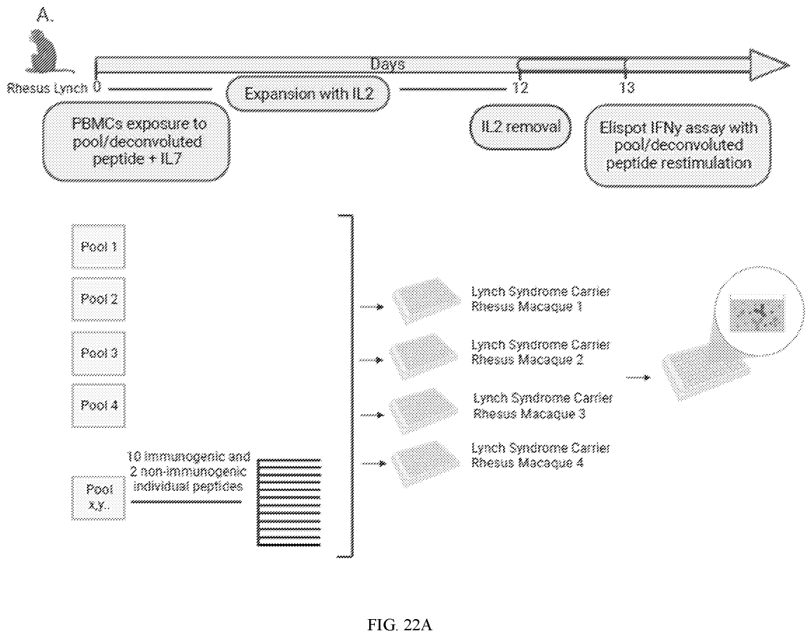

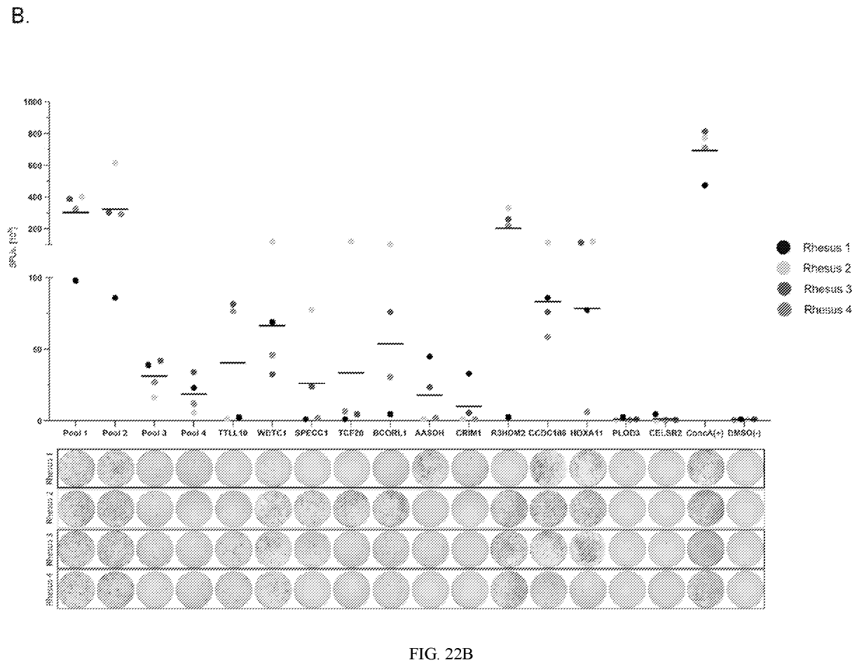

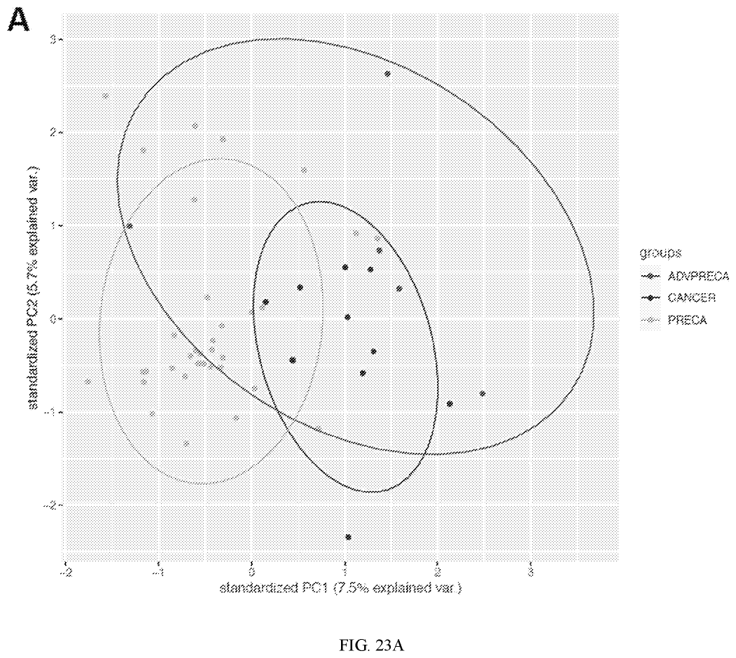

Since the ultimate goal is to develop a universal LS-cancer vaccine. the inventors then tested the immunogenicity of the ELISpot-reactive peptides in PBMCs from LS Rhesus macaques who carry a pathogenic mutation in the MLH1 gene with pathology similar to human LS. These animals. housed at MD Anderson Cancer Center, spontaneously develop CRC and serve as an ideal preclinical LS model for immune-interception strategies with neoAg vaccines. PBMCs from four different LS Rhesus were stimulated with four peptide pools and 12 individual peptides for ELISpot assays as indicated in FIG. 22A. These results demonstrate that all four donor PBMCs produced 80-600 SFUs in peptide pools 1 and 2 compared to DMSO control cells suggesting that pools 1 and 2 are immunogenic based on ELISpot assays (FIG. 22B). Further ELISpot assays with individual peptides showed that predicted neoAg derived from TTL10, WDTC1, SPECC1, BCORL1, AASDH, R3HDM2, CCDC186, and HOXA11 (8 out of 12) appeared to be highly immunogenic as they produced SFUs in the range of 25-400 per 10⁵ cells in at least two or more donors. These results, together with the human in-vitro validation, proves that the computational pipeline shows an excellent performance in predicting MHC-I neoAgs.

### 7. Transcriptomic Landscape and Immune Cell Profiles

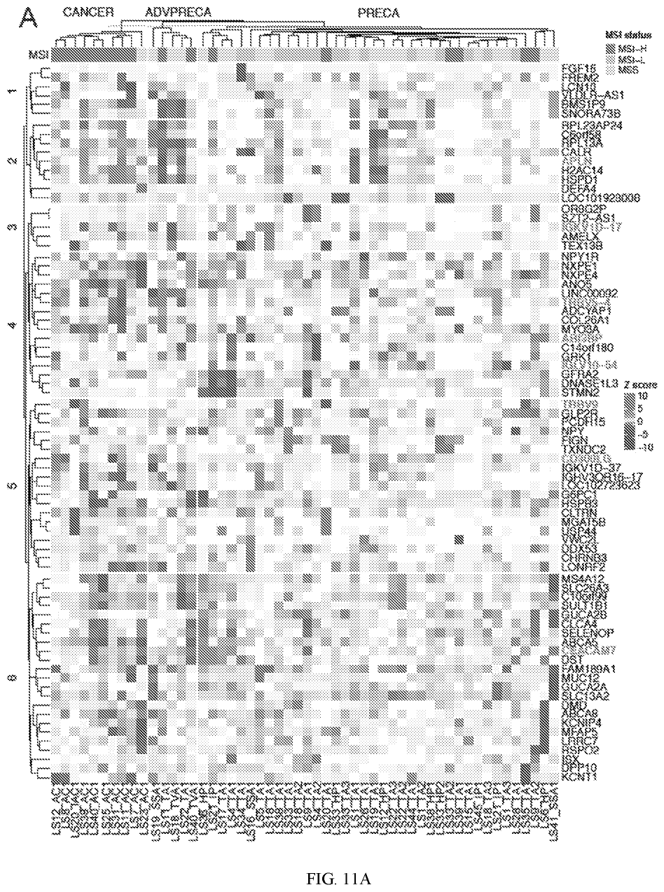

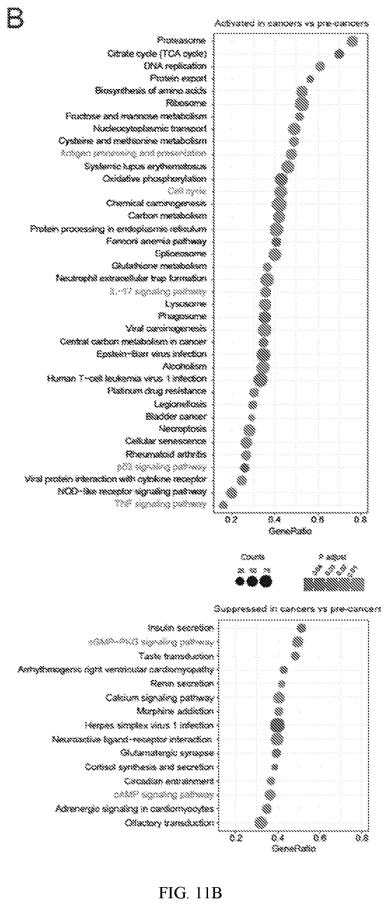

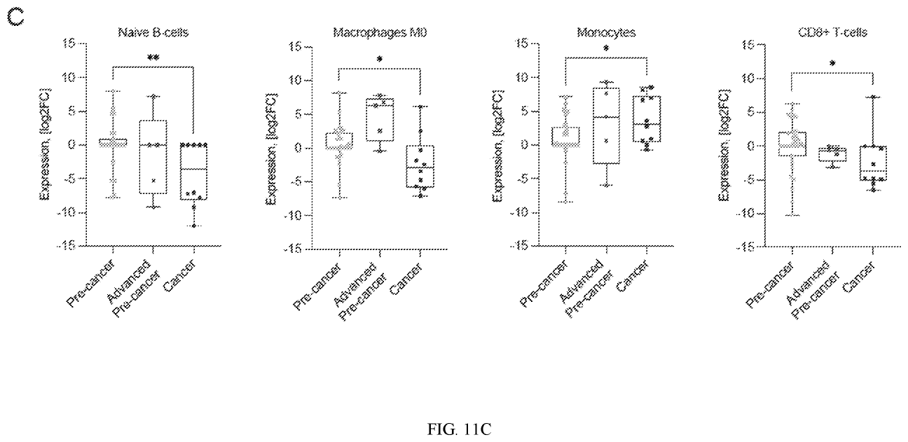

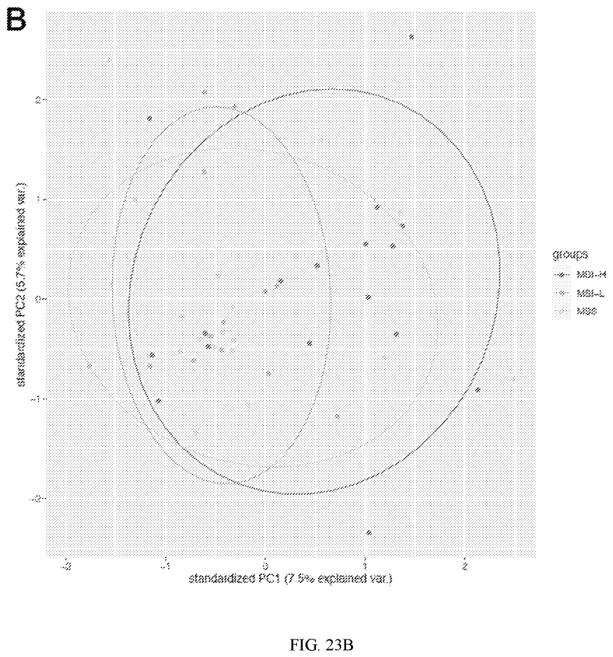

Given the increased number of neoags produced in cancers and certain precancers, the inventors decided to assess the level of immune activation at a transcriptomic level using the mRNAseq data in both the discovery and validation cohorts. Unsupervised clustering analysis showed a more effective grouping of the samples when the tissue category was considered compared to the MSI status (FIGS. 23A and B). The inventors found 78 genes to be significantly dysregulated between cancers and precancers (FIG. 11A), out of which seven were genes involved in immune responses (APLN, IGKVID-17, TRBV5-4, ABI3BP, IGLV10-54, TRBV9, CD300LG, CEACAM7). Interestingly, most of these immune genes were downregulated in cancers compared to precancers. However, after performing pathway enrichment analysis, the inventors found an activation of the antigen processing and presentation pathway, the IL-17 signaling pathway, the TNF signaling pathway, and several other pathways (FIG. 11B). In terms of immune cell infiltration, the inventors found naïve B-cells, macrophages MO, and CD8+ T-cells to be significantly decreased in cancers compared to precancers, while monocytes were significantly increased (FIG. 11C). Altogether, these results may potentially be explained by the fact that LS patients have a genetic predisposition to dMMR in all cells of their organism, which may warrant higher immune infiltration in normal tissue leading to a more pronounced immune response to tumor development, even at early stages (precancers and MSS stages). This level of surveillance is potentially decreased in MSI-H and cancerous stages due to these cells' capacity for immune evasion and subversion (13).

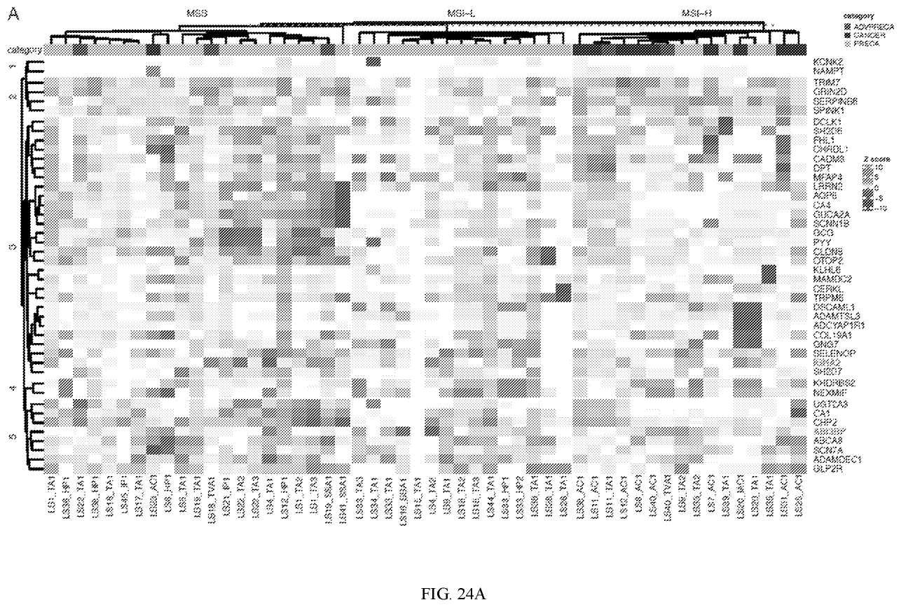

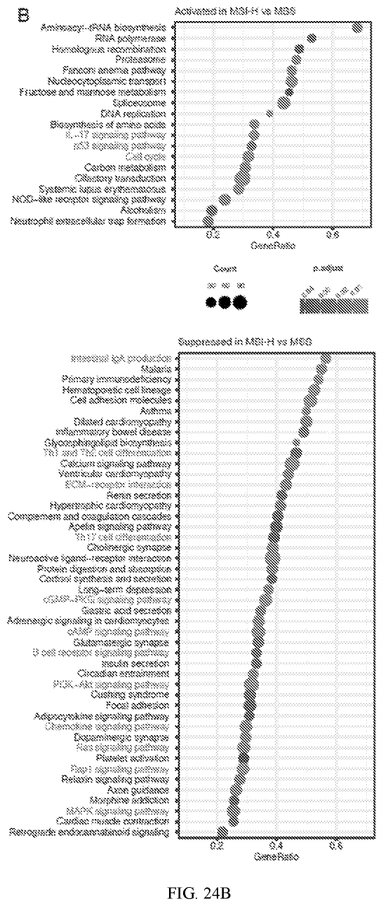

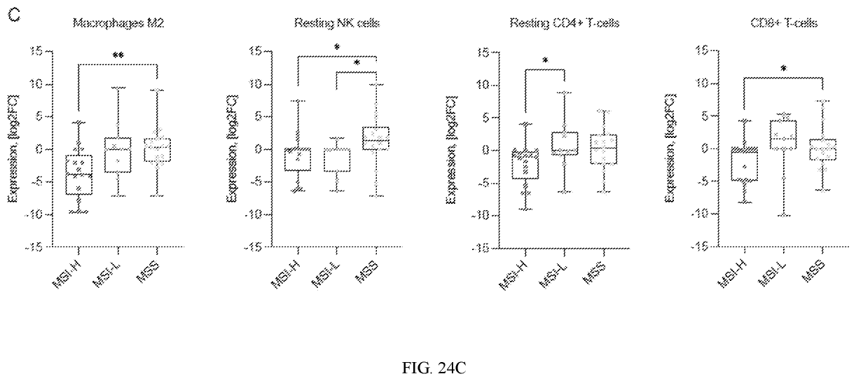

The inventors further confirmed this hypothesis by performing differential gene expression analysis between the MSI-H and MSS samples. The inventors observed fewer genes significantly dysregulated in MSI-H versus MSS (44 genes) compared to cancer versus precancer analysis. Only two out of 44 genes were involved in immune responses (IGHA2 and ABI3BP), which showed downregulation in MSI-H samples (FIG. 24A). Furthermore, IL-17, p53, and cell cycle signaling pathways were enriched in MSI-H samples. However, compared to the cancer versus precancer analysis, many more pathways, including chemokine signaling pathway, B-cell receptor signaling pathway, intestinal IgA production, and others involved in immune responses, were suppressed in MSI-H samples (FIG. 24B). After immune cell deconvolution, the inventors found resting NK cells and CD8+ T-cells significantly higher in MSS samples than MSI-H samples. Also, resting CD4+ T-cells were significantly higher in MSI-L samples compared to MSI-H (FIG. 24C).

### C. Discussion

Tumor development in the context of LS is characterized for dMMR, MSI, and the generation of high loads of neoags, that can be recognized by the host's immune system. LS patients are a defined population with high risk of cancer development at young ages, especially CRC. This makes them a distinctive group of individuals in which to assess preventive cancer vaccines. For this, some efforts have been made towards the identification of cancer-derived epitopes and their vaccine potential (9). Despite this, there is still big room for the improvement of in-silico prediction of candidate neoags with coupled in-vitro validation of immunogenicity, especially in MSI cancers and precancers.

In this study, the inventors used an approach that combines WES and mRNAseq data to identify a catalog of immunogenic and recurrent indel-derived MHC-I and II restricted neoags from a cohort of CRCs and precancers from LS patients routinely followed at MDACC. Different system biology platforms have been developed using next-generation sequencing (NGS) paired with bioinformatics pipelines to predict and catalog tumor-associated antigens from synonymous and nonsynonymous mutations as foreign antigens (neoantigens) to the host immune system (15, 16). However, accurately predicting the immunogenic frameshift peptides in coding microsatellite (cMS) mutations of the homopolymeric stretches in MSI cancers has always been challenging for computational analyses (17). These concerns are attributed mainly to the limited sensitivity of short-read next-generation approaches and ambiguities in alignment and assembly of the short repetitive DNA that produces biases and errors for accurately predicting FSP neoantigens in cMS mutations (18, 19). To address some of these unmet challenges, the pipeline incorporates mutation calling by combining the output from two different tools, Mutect2 (33) and MsMutect (34). MsMutect, in particular, allows for careful re-alignment of reads that contain MSs and nominates MS indels by applying an empirical noise profile based on motifs and the length of the repetitive DNA sequences. This way the rate of false-positive MS indel calls is significantly decreased and neoags are more accurately predicted (34).

The in-silico prediction and in-vitro validation identified a set of recurrent and immunogenic neoantigens generated in previously reported MS hotspots of genes that include RNF43, SEC31A, and ASTE1 (9), as well as novel MS hotspots that include BCORL1, TTLL10, R3HDM2, CRIM1, WDTC1, USP9Y, HOXA11, UBR5, SPINK5, among others.

To strengthen the predictive certainty, the inventors tested the strength of the pipeline again the TESLA benchmark (12), where the inventors observed that 25% of the predicted top 100 most immunogenic MHC-I neoAg met all presentation criteria and 13% met all five presentation and recognition criteria. The prediction pipeline exceeded the performance cutoffs established in the TESLA analysis of 10% for predicted peptides that passed presentation criteria and the 5% that passed the recognition criteria (12). These results suggest the pipeline, generating a prediction with 25% of the most immunogenic neoAgs meeting presentation criteria and 13% meeting the recognition criteria, has a strong performance compared to most pipelines analyzed by TESLA.

Importantly, the in-vitro immunogenicity assessment of 154 predicted neoAgs (MHC-I and MHC-II) with implementation of large-scale ELISpot assays using peptide pools and individual peptides, allowed us to identify several predicted neoAgs being highly immunogenic in PBMCs from healthy humans and LS Rhesus macaques. Even though, a few previous reports have demonstrated the in vitro immunogenicity of neoAg in MSI tumors, the numbers of neoags selected for this validation has been of relatively smaller scale, compared to this study (25, 26). One of the in vitro validation limitations was the exclusion of LS patient PBMCs due to specimen unavailability for conducting ELISpot assays, which could be considered for future experiments. Though utilizing PBMC with LS Rhesus macaques showed a higher reactivity of neoAg peptides than human PBMCs in ELISpot assays, thus validating the in vitro immunogenicity of these antigenic peptides. LS model of Rhesus macaques, carrying a pathogenic mutation in MLH1, similar to human LS, thus provides an ideal preclinical animal model for immune-interception strategies with neoAg peptide vaccination.

The utility of immune interception strategies as a cancer preventive has gained significant traction in recent years. The inventors recently reported a phase 1b clinical trial study on long-term exposure of naproxen, a non-steroidal anti-inflammatory drug (NSAID) to LS patients and observed that naproxen exposure led to an increase of resident immune cells within the mucosal tissue of the colon (27). Thus, it is plausible to posit that immune stimulators, such as naproxen, may amount a favorable immune response in mucosal tissues when combined with neoantigens vaccines, which would yield a durable immunoprevention for LS cancers, including CRC, EC, and other GI cancers. Further studies are needed to corroborate this hypothesis. This data demonstrates the powerful approaches of bioinformatics to identify, predict, and rank candidate neoantigens for future development of LS-specific immunotherapies.

In summary, the inventors report a novel, validated computational algorithm that predicts the immunogenicity and recurrency of frameshift neoantigen mutations in a cohort of LS patients routinely cared for at MDACC. This pipeline affords the ability to accurately identify candidate neoAgs suitable for developing cancer prevention vaccines and other durable interception modalities, which remains a huge unmet clinical need in the field of oncology.

### D. Methods

### 1. Patients and Specimen Collection

All patients included in this study had a confirmed diagnosis of Lynch Syndrome (n=46) and provided written informed consent at The University of Texas MD Anderson Cancer Center (MDACC). All samples were obtained from study participants through an Institutional Review Board (IRB) approved protocol at MDACC (Protocol PA12-0327). A set of 74 flash-frozen or formalin-fixed paraffin embedded (FFPE) tissue biopsies from polyps or tumors of the lower gastrointestinal tract, with matching normal mucosa and peripheral blood, were collected from 46 LS patients who came to MDACC for a standard of care surveillance colonoscopy (Supplementary Table 2). The pathological diagnosis of all tissue samples was confirmed by a gastrointestinal pathologist (M.W.T) at MDACC. DNA and RNA were extracted from flash-frozen and FFPE tissue samples using the Quick-DNA/RNA Miniprep Kit (Zymo Research, CA) and AllPrep DNA/RNA FFPE Kit (Qiagen, MD), respectively. Genomic DNA was obtained from peripheral blood using Gentra Puregene Blood Kit (Qiagen).

2. Whole-Exon Sequencing, mRNA Sequencing and Bioinformatics Analysis

Library construction and sequencing were performed at the MDACC Advanced Technology Genomics Core and the MDACC Cancer Genomics Laboratory. Samples were grouped into a discovery set and a validation set (Supplementary Table 4). RNA and DNA samples obtained from polyps, tumors, and matching normal mucosa were sequenced on a HiSeq4000 sequencer (Illumina) and NovaSeq 6000 sequencer (Illumina), respectively, for the discovery and the validation set. Alignment of WES data was performed using BWA mem 0.7.17 with default parameters to human genome reference hg19 [arXiv:1303.3997, Li, 2013]. Duplicate reads were marked with Picard 2.9.0 [Picard Toolkit.” 2018. Broad Institute, GitHub Repository [http://broadinstitute.github.io/picard/]. Base quality recalibration was performed with GATK Apply BQSR 4.1.2.0 (28). Alignment to human genome reference hg19 of mRNAseq data was performed using STAR (29) and bowtie 1.2.2.

### 3. Determination of MSI Status

MSIsensor was used to predict MSI status from WES data in both the discovery and validations sets as described previously (30). Duplicate reads were physically removed from normal and tumor BAM files using samtools (31, 32). Microsatellite loci in the hg19 reference genome were first identified by MSIsensor scan (30). The distribution for expected (normal) and observed (tumor) lengths of repeated sequence per microsatellite after coverage normalization was compared using Pearson's Chi-Squared Test, for which a default FDR=0.05 was used as a cutoff to identify somatic microsatellite sites by MSIsensor msi (30). MSIscore was defined as the percentage of somatic sites over a total number of microsatellite sites with minimal coverage of 20 in normal and paired tumor samples. Samples were then classified as MSI-H if MSIscore >=10%, as MSI-L if MSIscore <10% and >=3.5%, as MSS if MSIscore <3.5% based on the recommended cutoffs from MSIsensor (30). The number of somatic and non-somatic sites that passed the threshold of all samples were plotted as stacked barplot. MSIscore based sample classifications were indicated as the covariate bar in the waterfall plot.

### 4. Somatic Mutation Detection

Somatic mutations from both the discovery and validation sets were detected using Mutect2 4.0.8.1 following GATK best practices [(33). MSMuTect (34) was used for identifying somatic INDELs at microsatellite loci identified in hg19 reference genome by Phobos with default parameters [Mayer, Christoph, Phobos 3.3.11, 2006-2010, <found on the world wide web at: rub.de/ecoevo/cm/cm_phobos.htm>]. After MS-specific alignment, alleles were inferred by empirical noise model followed by mutation calling using Akaike information criterion (AIC) and Kolmogorov-Smirnov (KS)-test. With the default cutoff of MSMuTect, somatic mutations passing the threshold were annotated by Oncotator (35) and used as a part of the input for neoantigen discovering pipeline, together with the mutations detected using Mutect2.

### 5. Transcriptomics Analysis

All samples from the discovery and validation cohorts were included in the analysis, except for those which did not have paired normal tissue. Quality control of RNAseq results was assessed by the FASTQC software Ver. 0.11.5 (36). Adaptors and low-quality bases were trimmed using Trimmomatic Ver. 0.39 (37) with default parameters. Reads were mapped using Spliced Transcripts Alignment to a Reference (STAR Ver. 2.7.9a) (29) and counted using the RNA-Seq by Expectation Maximization (RSEM Ver. 1.3.1) (38). The raw counts were normalized by the trimmed mean of M values method (39). Differentially expressed genes (DEGs) were determined by the genewise negative binomial generalized linear model with quasi-likelihood test in the EdgeR package Ver. 3.36.0 (40) with the 0.05 as the Log 2FC and Benjamini-Hochberg (BH) adjusted P-value cut-off. The normalized counts per million (CPMs) of each sample were used to perform cellular deconvolution in the CIBERSORT-abs algorithm (41) implemented by the Immunedeconv package Ver. 2.0.4 (42). KEGG pathways' gene set enrichment analysis (43) of Kyoto encyclopedia of genes and genomes pathways (KEGG) (44) was preform by ClusterProfiler Ver. 4.0.5 (45). Batch-effect corrected DEGs were visualized by ComplexHeatmap Ver. 2.8.0 (46)

### 6. HLA Typing

MHC class I and II HLA alleles of each patient were detected from WES data using PHLAT with default settings (47).

### 7. Bioinformatic Approaches for the Neoantigens Prediction

Germline mutations were detected with GATK HaplotypeCaller 4.1.2.0 following GATK best-practice with SNV sensitivity threshold=99.9 and indel sensitivity threshold=98.0 (28) [biorxiv: 201178v2, Poplin, 2017]. Somatic mutations passed by Mutect2 and MsMutect were annotated by VEP version 98.3 (48). Somatic and germline mutations were phased using GATK ReadBackedPhasing 3.8. Corresponding RNA depth and variant allele frequency (VAF) of somatic mutations were collected with bam read count helper [https://github.com/genome/bam-readcount]. The inventors ran pVACseq 1.5.3 (7) to generate neoantigen predictions on phased somatic mutations and sample-specific MHC class I and II HLA alleles using the NetMHCpan 4.0 with epitope length of 8, 9, 10, 11 amino acids for MHC I peptides, and NetMHCIIpan 3.2 with epitope length of 15 for MHC II peptides (49, 50). Predicted neoantigens with binding affinity >500 nM and DNA VAF <0.05 were removed. Each predicted neoantigens was assigned an immunogenicity score which was obtained by combining the composite of the HLA binding affinity score, the binding score fold change rank, derived by dividing the binding affinity score of the wild-type protein by the neoAg; the allele expression rank (Tumor RNA variant allele frequency * gene expression in transcripts per million), the Tumor DNA variant allele frequency of each neoAg, and the Non-NA features, which are those neoAgs that did not have measurements for all the previous variables (FIG. 143). The binding stability of each predicted epitope was calculated using NetMHCStabPan with default parameters (51).

8. Selection and Preparation of neoAg Peptides for In Vitro Validation

Using the predicted immunogenicity score, the best-ranked neoAgs generated from each mutation across all samples from the discovery set were filtered, and 154 of those were selected based on their predicted immunogenicity score (Most Immunogenic) and their recurrency within the sample cohort (Most Recurrent) (FIG. 20). Ten neoAgs were randomly selected from the top 100 MHC Class I binder neoAgs with the highest immunogenicity score (Supplementary Table 5), even if those were not recurrent. Fifty-five neoAgs were randomly selected from the top 100 MHC Class I binder neoAgs with the highest recurrency within the sample cohort (Supplementary Table 6), even if these were not predicted to be highly immunogenic. Fourteen were MHC-I neoAgs from both the top 100 most immunogenic and top 100 most recurrent lists. Twenty neoAgs were selected from the top 100 MHC Class II neoAgs with the highest immunogenicity score (Supplementary Table 7), 17 were selected from the top 100 MHC Class II neoAgs with the highest recurrency within the sample cohort (Supplementary Table 8), and 7 were MHC-II neoAgs from both the top 100 most immunogenic and top 100 most recurrent lists. Finally, 31 MHC-I neoAgs with low predicted immunogenicity scores and no recurrency were also selected for validation (Supplementary Table 9). All selected peptides were synthesized by GenScript Biotech with purity >95%. Peptides were randomly grouped into 15 pools (Supplementary Table 13).

9. Culture and Expansion of T cells and ELISpot Assay

Healthy donor PBMCs were cultured on a 12-well plate (1.5×10⁶/well) in R10 media [(RPMI 1640 with L-glutamine (Cat #10040CV, Corning), 10% heat-inactivated FBS (Cat #SH30070.03, HyClone), 10 mM Hepes buffer (Cat #25060-CI, Corning), and 1× pen/strep (Cat #30002CI, Corning)] supplemented with recombinant human IL-7 (R&D Systems Biotechne, 330 U/ml). PBMCs were stimulated with the peptide pool or individual peptides (5 μg/ml each peptide individually or within pools). Concavaliin A and DMSO were used as positive and negative controls, respectively. On days 3, 7 and 10 of culture, cells were fed with R10 media in the presence of IL-2 (R&D Systems Biotechne, 20 U/mL). On day 12, cells were harvested and left for rest in R10 media overnight, at 37° C. On day 13, cells were seeded in triplicate (1×10⁵/well) onto a 96-well ELISpot plate (Mabtech Cat #3420-2apt-10) pre-coated with human IFNγ antibody. Cells were re-stimulated with the respective peptide pool or individual peptide (each peptide at 3 μg/mL) and cultured for 16-20 h. Where indicated, cells were also stimulated with Concavalin A (Invitrogen, 0.25 μg/mL) as a positive control. Following incubation, secreted IFN-γ was detected as per the manufacturer's instructions (Mabtech), and SFU cells were measured using an ImmunoSpot S6 UNIVERSAL analyzer (Cellular Technology Limited, OH). The inventors performed spot count normalization to account for cell concentration differences by factoring all counts to spots per 1×10⁵ cells. To determine the immunogenicity of peptide pools or individual peptides, the inventors performed ELISpot assays in six healthy donors (n=6) obtained from Stemcell Technologies Inc (Catalog #70025.3). Immunogenic pools were defined as those which produced ≥ 15 spot forming unit (SFUs) compared to the negative control (DMSO), after averaging the results from all donors.

### 10. Pan T Cells Isolation and Expansion

Untouched T cells (>96% purity) were isolated from PBMCs of HLA-A*02:01-positive healthy human donors using the Pan T Cell Isolation kit from Miltenyi Biotec (Bergisch Gladbach, Germany). Briefly, non-T cells were depleted from PBMC using biotin-conjugated Abs to CD14, CD16, CD19, CD36, CD56, CD123, and glycophorin A anti-biotin-labeled magnetic beads and LS columns. After isolation, 10×106 cells were cultured with Opto™ Antigen-Presenting Bead (Berkeley Lights, Emeryville, CA, USA) conjugated with WDTC1 neoAg peptide in advanced RPMI supplemented with 10% FBS, 1% GlutaMAX, 1% penicillin/streptomycin (Thermo Fisher Scientific), 55 nM of 2-mercaptocthanol (Sigma-Aldrich) and 30 ng/ml IL-21 (Cat no: 8879-IL-010) (R&D Systems) for 3 days. On day 3, a final 150 ng/ml IL-21 concentration was added and cultured for 5 days. On day 8 frequency of WDTC1-specific CD8+ T cells were analyzed by CYTOFLEX SRT Flow cytometer (Beckman Coulter, USA).

### 11. Flow Cytometry Analysis and Cell Sorting

For each healthy human donor, expanded Pan T cells were suspended in Ca2+ Mg2+ free Phosphate Buffered Saline (PBS), supplemented with 0.5% bovine serum albumin (BSA) (wash buffer), and were stained with R-phycoerythrin (PE)-labeled multimeric Pro5 pentamer HLA-A*02:01/FLADSGIDPV (Proimmune) and Peridinin-Chlorophyll-Protein (PerCP) Mouse Anti-Human CD8 antibody (cat no. 347314) (BD Biosciences, San Jose, CA, USA) to determine the number of WDTC1 neoAg peptide-specific CD8+ T cells. Cells were incubated for pentamer staining for 10 min in the dark on RT (22° C.) at the manufacturer's recommended concentrations. After the pentamer staining, cells were washed twice with 2 ml of wash buffer, centrifuged at 1,200 rpm for 5 min at 4° C., resuspended on the residual volume, and incubated with the anti-CD8 antibody for 20 min on ice. Dead cells were excluded by Sytox Blue staining (1 mM, Molecular Probes, Carlsbad, CA, USA). Unstained Pan-T cells were used to detect auto-fluorescence or background staining. Stained cells were analyzed and sorted using a CytoFLEX SRT Flow cytometer (Beckman Coulter, USA) under sterile conditions, and the results were analyzed by FlowJo Software version 10.8.1 (Tree Star, Inc., Ashland, OR, USA).

### 12. Statistical Analyses

Statistical analyses were performed using PRISM8. Non-parametric Mann-Whitney two-tailed test was used to infer the statistical significance of the differences between tissue categories and MSI status in terms of MSI score (FIG. 12B), mutational rate (FIG. 7B, and FIG. 132), numer of neoAgs (FIG. 8A) and immune cell expression (FIG. 11C and FIG. 24C). Non-parametric Spearman's rank correlation coefficient was used to infer the statistical significance of the correlation between mutational rate and number of neoAgs (FIG. 7B). For every test, significance was defined by a P value <0.05.

### E. Tables

TABLE 1
Summary of patient demographics and lesions characteristics.

| Characteristic | N | % |
|---|---|---|
| Age mean ± SD | 52 ± 15 | N/A |
| Gender | | |
| Female | 27 | 58.7 |
| Male | 19 | 41.3 |
| Race | | |
| African american | 4 | 8.6 |
| Asian | 2 | 4.4 |
| Caucasian | 32 | 69.6 |
| Other | 7 | 15.2 |
| Not disclosed | 1 | 2.2 |
| Ethnicity | | |
| Not hispanic or latino | 34 | 73.9 |
| Hispanic or latino | 9 | 19.6 |
| Not disclosed | 3 | 6.5 |
| dMMR gene | | |
| MLH1 | 11 | 23.9 |
| MSH2 | 14 | 30.4 |
| MSH6 | 14 | 30.4 |
| PMS2 | 5 | 10.9 |
| Not detected | 2 | 4.4 |
| Cancer Status | | |
| History of Cancer | 10 | 21.7 |
| No History of Cancer | 36 | 78.3 |
| Cancer at Diagnosis* | | |
| Colon | 19 | N/A |
| Endometrial | 7 | N/A |
| Rectum | 3 | N/A |
| Urothelial tract | 2 | N/A |
| Lung | 2 | N/A |
| Vulva | 1 | N/A |
| Prostate | 2 | N/A |
| Promyelocitic Leukemia | 1 | N/A |
| Uterus | 1 | N/A |
| Ureter | 1 | N/A |
| Melanoma | 1 | N/A |
| Sarcoma | 1 | N/A |
| Ovarian | 1 | N/A |
| Breast | 1 | N/A |
| Bladder | 1 | N/A |
| Colorectal Neoplasm* | | |
| Inflammatory polyp | 2 | N/A |
| Hyperplastic polyp | 8 | N/A |
| Tubular adenoma | 45 | N/A |
| Tubulovillous adenoma | 2 | N/A |
| Sessile serrated adenoma | 4 | N/A |
| Adenocarcinoma In Situ | 2 | N/A |
| Adenocarcinoma Stage I | 4 | N/A |
| Adenocarcinoma Stage II | 2 | N/A |
| Adenocarcinoma Stage III | 5 | N/A |
| Premalignant lession size (mm mean) ± SD | 22.6 ± 75.4 | N/A |

*One patient can have more than one cancer or neoplasm

SUPPLEMENTARY TABLE 1
Clinical, pathological, and demographical characteristics of each specimen collected from the LS patients included in this study.

| | | | | | Size | Tissue | MSIsensor | MSI |
|---|---|---|---|---|---|---|---|---|
| Biopsy_ID | Pathology | Category | Dysplasia | Location | (mm) | type | Score | status |
| LS1_TA1 | Tubular adenoma | Precancer | Low grade | Descending colon | 7 | FFPE | 0.04 | MSS |
| | | | dysplasia | | | | | |
| LS1_TA2 | Tubular adenoma | Precancer | No dysplasia | Ascending colon | 4 | FFPE | 0.04 | MSS |
| LS1_TA3 | Tubular adenoma | Precancer | No dysplasia | Descending colon | 7 | FFPE | 0.05 | MSS |
| LS2_TA1 | tubular adenoma | Precancer | Low grade | Transverse Colon and | 3 | FFPE | 0.11 | MSS |
| | | | dysplasia | sigmoid | | | | |
| LS3_AC1 | Adenocarcinoma Stage III | Cancer | Not applicable | Sigmoid | 103 | FFPE | 12.6 | MSI-H |
| LS3_AC2 | Adenocarcinoma Stage III | Cancer | Not applicable | sigmoid colon | 200 | FFPE | 5.24 | MSI-L |
| LS3_TA1 | Tubular adenoma | Precancer | Low grade | Transverse | 12 | FFPE | 14.21 | MSI-H |
| | | | dysplasia | | | | | |
| LS3_TA2 | Tubular adenoma | Advanced | High grade | Sigmoid | 8 | FFPE | 8.68 | MSI-L |
| | | Precancerncer | dysplasia | | | | | |
| LS3_TA3 | Tubular adenoma | Precancer | Low grade | Rectum | 8 | FFPE | 1.58 | MSI-L |
| | | | dysplasia | | | | | |
| LS4_TA1 | Tubular Adenoma | Precancer | No dysplasia | Ascending colon | 4 | Flash | 1.47 | MSS |
| | | | | | | frozen | | |
| LS4_TA2 | Tubular Adenoma | Precancer | No dysplasia | Cecum | 2 | FFPE | 7.15 | MSI-L |
| LS5_TA1 | tubular adenoma | Precancer | No dysplasia | Transverse Colon | 2 | FFPE | 0 | MSS |
| LS6_HP1 | Hyperplastic polyp | Precancer | No dysplasia | Cecum | 3 | Flash | 0.62 | MSS |
| | | | | | | frozen | | |
| LS7_AC1 | Adenocarcinoma Stage III | Cancer | Not applicable | Distal rectum | 41 | FFPE | 24.25 | MSI-H |
| LS8_AC1 | Tubular Adenoma with | Cancer | High grade | Hepatic Flexre | 12 | FFPE | 33.67 | MSI-H |
| | HGD/Tumor in situ | | dysplasia | | | | | |
| LS9_TA1 | Tubular Adenoma | Precancer | No dysplasia | Sigmoid | 5 | Flash | 4.29 | MSI-L |
| | | | | | | frozen | | |
| LS9_TA2 | Tubular Adenoma | Precancer | No dysplasia | Sigmoid | 5 | FFPE | 21.47 | MSI-H |
| LS10_TA1 | Tubular adenoma | Precancer | Low grade | Descending colon | 2 | FFPE | 0.08 | MSS |
| | | | dysplasia | | | | | |
| LS11_AC1 | Adenocarcinoma Stage I | Cancer | Not applicable | Rectum | 140 | FFPE | 17.2 | MSI-H |
| LS11_TA1 | tubular adenoma | Advanced | High grade | Sigmoid colon and rectum | 3 | FFPE | 22.55 | MSI-H |
| | | Precancerncer | dysplasia | | | | | |
| LS12_AC1 | Adenocarcinoma Stage III | Cancer | Not applicable | Ascending colon | 16 | FFPE | 11.76 | MSI-H |
| LS12_HP1 | Hyperplastic polyp | Precancer | No dysplasia | Ascending colon | 2 | FFPE | 0.01 | MSS |
| LS13_TA1 | Tubular adenoma | Precancer | Low grade | Cecum | 2 | FFPE | 0.14 | MSS |
| | | | dysplasia | | | | | |
| LS14_TA1 | Tubular adenoma | Precancer | Low grade | Descending colon | 7 | FFPE | 16.95 | MSI-H |
| | | | dysplasia | | | | | |
| LS15_TA1 | Tubular Adenoma | Precancer | No dysplasia | Cecum | 2 | FFPE | 8.56 | MSI-L |
| LS16_SSA1 | Sesile Serrated Adenoma | Precancer | No dysplasia | Cecum | 8 | FFPE | 6.88 | MSI-L |
| LS17_TA1 | Tubular Adenoma | Precancer | No dysplasia | Hepatic Flexure | 3 | Flash | 1.09 | MSS |
| | | | | | | frozen | | |
| LS18_TA1 | Tubular adenoma | Precancer | No dysplasia | Transverse | 3 | FFPE | 0.18 | MSS |
| LS18_TA2 | Tubular Adenoma | Precancer | No dysplasia | Splenic flexure | 2 | FFPE | 7.85 | MSI-L |
| LS18_TA3 | Tubular Adenoma | Precancer | No dysplasia | Cecum | 3 | FFPE | 7.04 | MSI-L |
| LS18_TVA1 | Tubulovillous adenoma | Advanced | No dysplasia | Cecum | 6 | FFPE | 0 | MSS |
| | | Precancerncer | | | | | | |
| LS19_SSA1 | Sessile serated adenoma | Advanced | No dysplasia | Sigmoid | 100 | FFPE | 0.49 | MSS |
| | | Precancerncer | | | | | | |
| LS19_TA1 | tubular adenoma | Precancer | Low grade | Transverse Colon | 3 | FFPE | 0.09 | MSS |
| | | | dysplasia | | | | | |
| LS20_IAC1 | IMCA/Tumor in situ | Cancer | High grade | Transverse | 10 | FFPE | 12.64 | MSI-H |
| | | | dysplasia | | | | | |
| LS20_TA1 | Tubular Adenoma | Precancer | High grade | Transverse | 10 | FFPE | 28.98 | MSI-H |
| | | | dysplasia | | | | | |
| LS21_IP1 | Inflammatory polyp | Precancer | No dysplasia | Cecum | 2 | FFPE | 0.04 | MSS |
| LS22_TA1 | Tubular adenoma | Advanced | High grade | Ascending polyp/Hepatic | 12 | FFPE | 0.1 | MSS |
| | | Precancerncer | dysplasia | Flexure | | | | |
| LS22_TA2 | tubular adenoma | Precancer | No dysplasia | Ascending colon | 3 | FFPE | 0.04 | MSS |
| LS22_TA3 | Tubular adenoma | Precancer | High grade | Ascending colon | 3 | FFPE | 0.06 | MSS |
| | | | dysplasia | | | | | |
| LS23_AC1 | Adenocarcinoma Stage I | Cancer | No dysplasia | Transverse | 10 | Flash | 0.55 | MSS |
| | | | | | | frozen | | |
| LS24_TA1 | Tubular adenoma | Precancer | No dysplasia | Cecum | 3 | FFPE | 11.77 | MSI-H |
| LS25_AC1 | Adenocarcinoma Stage I | Cancer | Not applicable | Ascending colon | 4 | FFPE | 15.3 | MSI-H |
| LS26_TA1 | Tubular Adenoma | Precancer | No dysplasia | Cecum | 3 | FFPE | 8.27 | MSI-L |
| LS27_HP1 | Hyperplastic polyp | Precancer | No dysplasia | Sigmoid | 3 | FFPE | 0.04 | MSS |
| LS27_HP2 | Hyperplastic polyp | Precancer | No dysplasia | Sigmoid | 2 | FFPE | 6.93 | MSI-L |
| LS27_TA1 | Tubular Adenoma | Precancer | No dysplasia | Ascending colon | 2 | FFPE | 6.19 | MSI-L |
| LS28_TA1 | Tubular Adenoma | Precancer | No dysplasia | Transverse | 2 | Flash | 3.98 | MSI-L |
| | | | | | | frozen | | |
| LS29_AC1 | Adenocarcinoma Stage III | Cancer | Not applicable | Transverse Colon | 70 | FFPE | 0.01 | MSS |
| LS30_TA1 | Tubular adenoma | Precancer | Low grade | Ascending colon | 4 | FFPE | 0.03 | MSS |
| | | | dysplasia | | | | | |
| LS31_AC1 | Adenocarcinoma Stage II | Cancer | Not applicable | Ileocolic anastomosis | 40 | FFPE | 26.16 | MSI-H |
| LS32_TA1 | Tubular adenoma | Advanced | Low grade | Rectum | 10 | FFPE | 18.8 | MSI-H |
| | | Precancerncer | dysplasia | | | | | |
| LS33_HP1 | Hyperplastic polyp | Precancer | No dysplasia | Cecum and transverse | 2 | FFPE | 5.88 | MSI-L |
| LS33_TA1 | Tubular Adenoma | Precancer | No dysplasia | Cecum and transverse | 2 | FFPE | 5.24 | MSI-L |
| LS33_TA2 | Tubular Adenoma | Precancer | No dysplasia | Ascending colon | 5 | FFPE | 19.7 | MSI-H |
| LS33_TA3 | Tubular Adenoma | Precancer | No dysplasia | Descending colon | 3 | FFPE | 6.81 | MSI-L |
| LS33_TA4 | Tubular Adenoma | Precancer | No dysplasia | Descending colon | 3 | FFPE | 7.18 | MSI-L |
| LS34_TA1 | Tubular Adenoma | Precancer | No dysplasia | Descending colon | 3 | FFPE | 6.33 | MSI-H |
| LS35_TA1 | Tubular Adenoma | Precancer | No dysplasia | Transverse Colon | 5 | FFPE | 31.96 | MSI-H |
| LS36_HP1 | Hyperplastic polyp | Precancer | No dysplasia | Transverse Colon | 5 | Flash | 0.34 | MSS |
| | | | | | | frozen | | |
| LS37_TA1 | Tubular Adenoma | Precancer | No dysplasia | Cecum | 5 | FFPE | 8.08 | MSI-L |
| LS38_AC1 | Adenocarcinoma Stage II | Cancer | Not applicable | Right Colon | 600 | FFPE | 20.46 | MSI-H |
| LS38_HP1 | Hyperplastic polyp | Precancer | No dysplasia | Transverse | 4 | FFPE | 0.06 | MSS |
| LS38_TA1 | Tubular Adenoma | Precancer | No dysplasia | Transverse | 6 | FFPE | 8.33 | MSI-L |
| LS39_TA1 | Tubular Adenoma | Precancer | No dysplasia | Rectum | 5 | FFPE | 14.16 | MSI-H |
| LS40_AC1 | Adenocarcinoma Stage I | Cancer | Not applicable | Sigmoid | 30 | Flash | 29.9 | MSI-H |
| | | | | | | frozen | | |
| LS40_TVA1 | Tubulovillous Adenoma | Advanced | No dysplasia | Sigmoid | 30 | Flash | 29.75 | MSI-H |
| | | Precancer | | | | frozen | | |
| LS41_SSA1 | Sessile serated adenoma | Precancer | No dysplasia | Ascending colon | 3 | FFPE | 0.41 | MSS |
| LS42_TA1 | Tubular adenoma | Precancer | No dysplasia | Hepatic Flexure | 3 | FFPE | 0.93 | MSS |
| LS42_TA2 | Tubular adenoma | Advanced | No dysplasia | Ascending colon | 15 | FFPE | 0.02 | MSS |
| | | Precancerncer | | | | | | |
| LS43_HP1 | Hyperplastic polyp | Precancer | No dysplasia | Transverse | 3 | FFPE | 6.9 | MSI-L |
| LS44_TA1 | tubular adenoma | Precancer | Low grade | Rectum | 4 | FFPE | 6.36 | MSI-L |
| | | | dysplasia | | | | | |
| LS45_IP1 | Inflammatory polyp | Precancer | No dysplasia | Sigmoid | 4 | FFPE | 0.08 | MSS |
| LS46_SSA1 | Sessile serated adenoma | Precancer | No dysplasia | Descending colon | 5 | FFPE | 0.01 | MSS |
| LS46_TA1 | tubular adenoma | Precancer | No dysplasia | Cecum | 5 | FFPE | 0 | MSS |

| | | | | | | | History | |
|---|---|---|---|---|---|---|---|---|
| Biopsy_ID | Patient_ID | Age | Gender | Race | Ethnicity | Mutated Gene | of Cancer | Type of cancer |
| LS1_TA1 | LS1 | 55 | Female | Caucasian | Not hispanic or latino | MSH6 | Yes | Vulva |
| LS1_TA2 | | | | | | | | |
| LS1_TA3 | | | | | | | | |
| LS2_TA1 | LS2 | 43 | Female | Caucasian | Not hispanic or latino | MLH1 | Yes | Endometrial |
| LS3_AC1 | LS3 | 53 | Male | Caucasian | Not hispanic or latino | MSH2 | Yes | Colon |
| LS3_AC2 | | | | | | | | |
| LS3_TA1 | | | | | | | | |
| LS3_TA2 | | | | | | | | |
| LS3_TA3 | | | | | | | | |
| LS4_TA1 | LS4 | 70 | Male | Asian | Not hispanic or latino | MSH6 | Yes | Lung |
| LS4_TA2 | | | | | | | | |
| LS5_TA1 | LS5 | 36 | Female | Caucasian | Hispanic or latino | MLH1 | No | N/A |
| LS6_HP1 | LS6 | 46 | Female | Caucasian | Not hispanic or latino | MSH6 | No | N/A |
| LS7_AC1 | LS7 | 35 | Male | Caucasian | Not hispanic or latino | MLH1 | Yes | Rectum |
| LS8_AC1 | LS8 | 73 | Male | Caucasian | Not hispanic or latino | MSH2 | Yes | Colon, Lung, Melanoma |
| LS9_TA1 | LS9 | 39 | Female | Other | Hispanic or latino | MSH2 | Yes | Rectum |
| LS9_TA2 | | | | | | | | |
| LS10_TA1 | LS10 | 56 | Female | Caucasian | Not hispanic or latino | MSH6 | Yes | Endometrial |
| LS11_AC1 | LS11 | 68 | Female | Caucasian | Not hispanic or latino | MSH2 | Yes | Colon, Uterus, Ureter |
| LS11_TA1 | | | | | | | | |
| LS12_AC1 | LS12 | 43 | Female | Other | Hispanic or latino | MSH6 | Yes | Colon |
| LS12_HP1 | | | | | | | | |
| LS13_TA1 | LS13 | 59 | Female | Caucasian | Not hispanic or latino | MLH1 | Yes | Endometrial |
| LS14_TA1 | LS14 | 51 | Female | Other | Hispanic or latino | MLH1 | Yes | Colon |
| LS15_TA1 | LS15 | 60 | Female | African American | Not hispanic or latino | MSH6 | No | N/A |
| LS16_SSA1 | LS16 | 37 | Female | Asian | Not hispanic or latino | MSH2 | No | N/A |
| LS17_TA1 | LS17 | 72 | Male | African American | Not hispanic or latino | MSH6 | Yes | Colon, Sarcoma |
| LS18_TA1 | LS18 | 65 | Female | Caucasian | Not hispanic or latino | MSH2 | Yes | Colon |
| LS18_TA2 | | | | | | | | |
| LS18_TA3 | | | | | | | | |
| LS18_TVA1 | | | | | | | | |
| LS19_SSA1 | LS19 | 72 | Female | Caucasian | Not hispanic or latino | MSH6 | Yes | Promyelocitic Leukemia |
| LS19_TA1 | | | | | | | | |
| LS20_IAC1 | LS20 | 41 | Female | African American | Not hispanic or latino | PMS2 | Yes | Rectum |
| LS20_TA1 | | | | | | | | |
| LS21_IP1 | LS21 | 47 | Male | Caucasian | Not hispanic or latino | MSH2 | No | N/A |
| LS22_TA1 | LS22 | 77 | Female | Caucasian | Not hispanic or latino | PMS2 | Yes | Colon, Endometrial |
| LS22_TA2 | | | | | | | | |
| LS22_TA3 | | | | | | | | |
| LS23_AC1 | LS23 | 33 | Male | Other | Hispanic or latino | MLH1 | Yes | Colon |
| LS24_TA1 | LS24 | 63 | Female | Caucasian | Not hispanic or latino | MSH6 | Yes | Endometrial |
| LS25_AC1 | LS25 | 56 | Male | Caucasian | Not hispanic or latino | MLH1 | Yes | Colon |
| LS26_TA1 | LS26 | 73 | Male | Caucasian | Not hispanic or latino | MSH6 | Yes | Bladder |
| LS27_HP1 | LS27 | 24 | Male | Caucasian | Not hispanic or latino | Not detected | No | N/A |
| LS27_HP2 | | | | | | | | |
| LS27_TA1 | | | | | | | | |
| LS28_TA1 | LS28 | 67 | Male | African American | Not hispanic or latino | MSH6 | Yes | Colon, Prostate |
| LS29_AC1 | LS29 | 30 | Female | Unknown | Not hispanic or latino | MSH2 | Yes | Colon |
| LS30_TA1 | LS30 | 59 | Female | Caucasian | Not hispanic or latino | PMS2 | Yes | Endometrial |
| LS31_AC1 | LS31 | 50 | Male | Other | Hispanic or latino | MLH1 | Yes | Colon |
| LS32_TA1 | LS32 | 46 | Female | Caucasian | Not hispanic or latino | MSH2 | Yes | Ovarian, Breast |
| LS33_HP1 | LS33 | 80 | Female | Caucasian | Hispanic or latino | MSH2 | No | N/A |
| LS33_TA1 | | | | | | | | |
| LS33_TA2 | | | | | | | | |
| LS33_TA3 | | | | | | | | |
| LS33_TA4 | | | | | | | | |
| LS34_TA1 | LS34 | 51 | Female | Caucasian | Not hispanic or latino | MSH6 | Yes | Endometrial |
| LS35_TA1 | LS35 | 62 | Female | Caucasian | Not hispanic or latino | MSH2 | Yes | Colon |
| LS36_HP1 | LS36 | 53 | Female | Caucasian | Not hispanic or latino | Not detected | Yes | Rectum |
| LS37_TA1 | LS37 | 47 | Male | Other | Hispanic or latino | PMS2 | Yes | Colon |
| LS38_AC1 | LS38 | 48 | Male | Other | Hispanic or latino | MLH1 | Yes | Colon |
| LS38_HP1 | | | | | | | | |
| LS38_TA1 | | | | | | | | |
| LS39_TA1 | LS39 | 53 | Male | Caucasian | Not hispanic or latino | MLH1 | Yes | Colon |
| LS40_AC1 | LS40 | 20 | Male | Caucasian | Not hispanic or latino | PMS2 | Yes | Colon, Glioblastoma |
| LS40_TVA1 | | | | | | | | |
| LS41_SSA1 | LS41 | 68 | Male | Caucasian | Not hispanic or latino | MSH6 | Yes | Prostate |
| LS42_TA1 | LS42 | 57 | Female | Caucasian | Not hispanic or latino | MSH2 | Yes | Urothelial tract |
| LS42_TA2 | | | | | | | | |
| LS43_HP1 | LS43 | 52 | Male | Caucasian | Patient refused | MSH6 | Yes | Colon |
| LS44_TA1 | LS44 | 53 | Female | Caucasian | Not hispanic or latino | MSH2 | No | N/A |
| LS45_IP1 | LS45 | 28 | Male | Caucasian | Patient refused | MSH2 | No | N/A |
| LS46_SSA1 | LS46 | 40 | Female | Caucasian | Patient refused | MLH1 | No | N/A |
| LS46_TA1 | | | | | | | | |

Supplementary Table 2. Frequency of the different HLA
alleles that are present within the cohort of LS patients.

| Locus | Locus | N | % |
|---|---|---|---|
| HLA_A | A*02:01 | 15 | 32 |
| HLA_A | A*01:01 | 14 | 30 |
| HLA_A | A*03:01 | 9 | 19 |
| HLA_A | A*11:01 | 7 | 15 |
| HLA_A | A*24:02 | 5 | 11 |
| HLA_A | A*01:10 | 4 | 9 |
| HLA_A | A*01:22 | 4 | 9 |
| HLA_A | A*31:01 | 4 | 9 |
| HLA_A | A*33:01 | 4 | 9 |
| HLA_A | A*68:01 | 4 | 9 |
| HLA_A | A*01:81 | 3 | 6 |
| HLA_A | A*32:01 | 3 | 6 |
| HLA_A | A*02:06 | 2 | 4 |
| HLA_A | A*02:43 | 2 | 4 |
| HLA_A | A*11:50 | 2 | 4 |
| HLA_A | A*23:01 | 2 | 4 |
| HLA_A | A*26:01 | 2 | 4 |
| HLA_A | A*29:02 | 2 | 4 |
| HLA_A | A*33:03 | 2 | 4 |
| HLA_A | A*02:14 | 1 | 2 |
| HLA_A | A*03:12 | 1 | 2 |
| HLA_A | A*03:26 | 1 | 2 |
| HLA_A | A*30:01 | 1 | 2 |
| HLA_A | A*30:02 | 1 | 2 |
| HLA_A | A*69:01 | 1 | 2 |
| HLA_A | A*74:01 | 1 | 2 |
| HLA_B | B*07:02 | 16 | 34 |
| HLA_B | B*08:01 | 8 | 17 |
| HLA_B | B*14:02 | 6 | 13 |
| HLA_B | B*40:01 | 6 | 13 |
| HLA_B | B*51:01 | 6 | 13 |
| HLA_B | B*35:01 | 5 | 11 |
| HLA_B | B*58:01 | 4 | 9 |
| HLA_B | B*44:02 | 3 | 6 |
| HLA_B | B*45:01 | 3 | 6 |
| HLA_B | B*53:01 | 3 | 6 |
| HLA_B | B*13:02 | 2 | 4 |
| HLA_B | B*15:01 | 2 | 4 |
| HLA_B | B*18:01 | 2 | 4 |
| HLA_B | B*35:03 | 2 | 4 |
| HLA_B | B*44:03 | 2 | 4 |
| HLA_B | B*57:01 | 2 | 4 |
| HLA_B | B*14:01 | 1 | 2 |
| HLA_B | B*15:02 | 1 | 2 |
| HLA_B | B*15:03 | 1 | 2 |
| HLA_B | B*15:17 | 1 | 2 |
| HLA_B | B*15:22 | 1 | 2 |
| HLA_B | B*27:05 | 1 | 2 |
| HLA_B | B*35:08 | 1 | 2 |
| HLA_B | B*37:01 | 1 | 2 |
| HLA_B | B*40:02 | 1 | 2 |
| HLA_B | B*41:01 | 1 | 2 |
| HLA_B | B*42:01 | 1 | 2 |
| HLA_B | B*48:01 | 1 | 2 |
| HLA_B | B*54:01 | 1 | 2 |
| HLA_B | B*55:01 | 1 | 2 |
| HLA_B | B*73:01 | 1 | 2 |
| HLA_C | C*07:02 | 16 | 34 |
| HLA_C | C*04:01 | 12 | 26 |
| HLA_C | C*07:01 | 9 | 19 |
| HLA_C | C*06:02 | 7 | 15 |
| HLA_C | C*08:02 | 6 | 13 |
| HLA_C | C*03:04 | 5 | 11 |
| HLA_C | C*05:01 | 4 | 9 |
| HLA_C | C*15:02 | 4 | 9 |
| HLA_C | C*01:02 | 3 | 6 |
| HLA_C | C*03:02 | 3 | 6 |
| HLA_C | C*03:03 | 3 | 6 |
| HLA_C | C*16:01 | 3 | 6 |
| HLA_C | C*02:02 | 2 | 4 |
| HLA_C | C*02:10 | 2 | 4 |
| HLA_C | C*08:01 | 2 | 4 |
| HLA_C | C*12:03 | 2 | 4 |
| HLA_C | C*17:01 | 2 | 4 |
| HLA_C | C*03:46 | 1 | 2 |
| HLA_C | C*14:02 | 1 | 2 |
| HLA_C | C*15:05 | 1 | 2 |
| HLA_DQA1 | DQA1*01:02 | 23 | 49 |
| HLA_DQA1 | DQA1*05:01 | 13 | 28 |
| HLA_DQA1 | DQA1*01:01 | 12 | 26 |
| HLA_DQA1 | DQA1*02:01 | 9 | 19 |
| HLA_DQA1 | DQA1*05:05 | 7 | 15 |
| HLA_DQA1 | DQA1*01:03 | 5 | 11 |
| HLA_DQA1 | DQA1*01:05 | 4 | 9 |
| HLA_DQA1 | DQA1*03:01 | 4 | 9 |
| HLA_DQA1 | DQA1*03:03 | 3 | 6 |
| HLA_DQA1 | DQA1*04:01 | 3 | 6 |
| HLA_DQA1 | DQA1*01:04 | 2 | 4 |
| HLA_DQA1 | DQA1*03:02 | 1 | 2 |
| HLA_DQB1 | DQB1*06:02 | 17 | 36 |
| HLA_DQB1 | DQB1*05:01 | 15 | 32 |
| HLA_DQB1 | DQB1*02:01 | 10 | 21 |
| HLA_DQB1 | DQB1*03:01 | 7 | 15 |
| HLA_DQB1 | DQB1*02:02 | 6 | 13 |
| HLA_DQB1 | DQB1*03:03 | 5 | 11 |
| HLA_DQB1 | DQB1*06:03 | 5 | 11 |
| HLA_DQB1 | DQB1*06:04 | 5 | 11 |
| HLA_DQB1 | DQB1*03:02 | 4 | 9 |
| HLA_DQB1 | DQB1*03:19 | 4 | 9 |
| HLA_DQB1 | DQB1*04:02 | 4 | 9 |
| HLA_DQB1 | DQB1*05:03 | 2 | 4 |
| HLA_DQB1 | DQB1*06:09 | 2 | 4 |
| HLA_DQB1 | DQB1*05:02 | 1 | 2 |
| HLA_DRB1 | DRB1*15:01 | 16 | 34 |
| HLA_DRB1 | DRB1*03:01 | 10 | 21 |
| HLA_DRB1 | DRB1*07:01 | 9 | 19 |
| HLA_DRB1 | DRB1*01:01 | 8 | 17 |
| HLA_DRB1 | DRB1*13:02 | 7 | 15 |
| HLA_DRB1 | DRB1*13:01 | 5 | 11 |
| HLA_DRB1 | DRB1*01:02 | 4 | 9 |
| HLA_DRB1 | DRB1*10:01 | 4 | 9 |
| HLA_DRB1 | DRB1*11:01 | 3 | 6 |
| HLA_DRB1 | DRB1*04:03 | 2 | 4 |
| HLA_DRB1 | DRB1*04:04 | 2 | 4 |
| HLA_DRB1 | DRB1*08:04 | 2 | 4 |
| HLA_DRB1 | DRB1*09:01 | 2 | 4 |
| HLA_DRB1 | DRB1*11:04 | 2 | 4 |
| HLA_DRB1 | DRB1*15:03 | 2 | 4 |
| HLA_DRB1 | DRB1*01:03 | 1 | 2 |
| HLA_DRB1 | DRB1*03:17 | 1 | 2 |
| HLA_DRB1 | DRB1*04:01 | 1 | 2 |
| HLA_DRB1 | DRB1*04:05 | 1 | 2 |
| HLA_DRB1 | DRB1*04:07 | 1 | 2 |
| HLA_DRB1 | DRB1*08:01 | 1 | 2 |
| HLA_DRB1 | DRB1*08:02 | 1 | 2 |
| HLA_DRB1 | DRB1*08:03 | 1 | 2 |
| HLA_DRB1 | DRB1*12:01 | 1 | 2 |
| HLA_DRB1 | DRB1*13:03 | 1 | 2 |
| HLA_DRB1 | DRB1*13:04 | 1 | 2 |
| HLA_DRB1 | DRB1*14:02 | 1 | 2 |
| HLA_DRB1 | DRB1*14:54 | 1 | 2 |

Supplementary Table 3. Distribution of samples for the discovery and validation sets.

| Biopsy_ID | Sequencing Set | Pathology | Category | MSI status |
|---|---|---|---|---|
| LS3_AC1 | Discovery | Adenocarcinoma | Cancer | MSI-H |
| LS3_AC2 | Discovery | Adenocarcinoma | Cancer | MSI-L |
| LS11_AC1 | Discovery | Adenocarcinoma | Cancer | MSI-H |
| LS12_AC1 | Discovery | Adenocarcinoma | Cancer | MSI-H |
| LS25_AC1 | Discovery | Adenocarcinoma | Cancer | MSI-H |
| LS38_AC1 | Discovery | Adenocarcinoma | Cancer | MSI-H |
| LS31_AC1 | Discovery | Adenocarcinoma Stage II | Cancer | MSI-H |
| LS7_AC1 | Discovery | Adenocarcinoma Stage III | Cancer | MSI-H |
| LS29_AC1 | Discovery | Adenocarcinoma Stage III | Cancer | MSS |
| LS12_HP1 | Discovery | Hyperplastic polyp | Precancer | MSS |
| LS27_HP1 | Discovery | Hyperplastic polyp | Precancer | MSS |
| LS38_HP1 | Discovery | Hyperplastic polyp | Precancer | MSS |
| LS21_IP1 | Discovery | Inflammatory polyp | Precancer | MSS |
| LS45_IP1 | Discovery | Inflammatory polyp | Precancer | MSS |
| LS19_SSA1 | Discovery | Sessile serated adenoma | Advanced | MSS |
| | | | Precancerncer | |
| LS41_SSA1 | Discovery | Sessile serated adenoma | Precancer | MSS |
| LS46_SSA1 | Discovery | Sessile serated adenoma | Precancer | MSS |
| LS1_TA1 | Discovery | Tubular adenoma | Precancer | MSS |
| LS1_TA2 | Discovery | Tubular adenoma | Precancer | MSS |
| LS1_TA3 | Discovery | Tubular adenoma | Precancer | MSS |
| LS2_TA1 | Discovery | tubular adenoma | Precancer | MSS |
| LS3_TA1 | Discovery | Tubular adenoma | Precancer | MSI-H |
| LS3_TA2 | Discovery | Tubular adenoma | Advanced | MSI-L |
| | | | Precancerncer | |
| LS3_TA3 | Discovery | Tubular adenoma | Precancer | MSI-L |
| LS5_TA1 | Discovery | tubular adenoma | Precancer | MSS |
| LS10_TA1 | Discovery | Tubular adenoma | Precancer | MSS |
| LS11_TA1 | Discovery | tubular adenoma | Advanced | MSI-H |
| | | | Precancerncer | |
| LS13_TA1 | Discovery | Tubular adenoma | Precancer | MSS |
| LS14_TA1 | Discovery | Tubular adenoma | Precancer | MSI-H |
| LS18_TA1 | Discovery | Tubular adenoma | Precancer | MSS |
| LS19_TA1 | Discovery | tubular adenoma | Precancer | MSS |
| LS22_TA1 | Discovery | Tubular adenoma | Advanced | MSS |
| | | | Precancerncer | |
| LS22_TA2 | Discovery | tubular adenoma | Precancer | MSS |
| LS22_TA3 | Discovery | Tubular adenoma | Precancer | MSS |
| LS24_TA1 | Discovery | Tubular adenoma | Precancer | MSI-H |
| LS30_TA1 | Discovery | Tubular adenoma | Precancer | MSS |
| LS32_TA1 | Discovery | Tubular adenoma | Advanced | MSI-H |
| | | | Precancerncer | |
| LS42_TA1 | Discovery | Tubular adenoma | Precancer | MSS |
| LS42_TA2 | Discovery | Tubular adenoma | Advanced | MSS |
| | | | Precancerncer | |
| LS44_TA1 | Discovery | tubular adenoma | Precancer | MSI-L |
| LS46_TA1 | Discovery | tubular adenoma | Precancer | MSS |
| LS8_AC1 | Discovery | Tubular Adenoma with | Cancer | MSI-H |
| | | HGD/Tumor in situ | | |
| LS18_TVA1 | Discovery | Tubulovillous adenoma | Advanced | MSS |
| | | | Precancerncer | |
| LS23_AC1 | Validation | Adenocarcinoma | Cancer | MSS |
| LS40_AC1 | Validation | Adenocarcinoma | Cancer | MSI-H |
| LS20_IAC1 | Validation | HGD/IMCA | Cancer | MSI-H |
| LS6_HP1 | Validation | Hyperplastic polyp | Precancer | MSS |
| LS27_HP2 | Validation | Hyperplastic polyp | Precancer | MSI-L |
| LS33_HP1 | Validation | Hyperplastic polyp | Precancer | MSI-L |
| LS36_HP1 | Validation | Hyperplastic polyp | Precancer | MSS |
| LS43_HP1 | Validation | Hyperplastic polyp | Precancer | MSI-L |
| LS16_SSA1 | Validation | Sesile Serrated Adenoma | Precancer | MSI-L |
| LS4_TA1 | Validation | Tubular Adenoma | Precancer | MSS |
| LS4_TA2 | Validation | Tubular Adenoma | Precancer | MSI-L |
| LS9_TA1 | Validation | Tubular Adenoma | Precancer | MSI-L |
| LS9_TA2 | Validation | Tubular Adenoma | Precancer | MSI-H |
| LS15_TA1 | Validation | Tubular Adenoma | Precancer | MSI-L |
| LS17_TA1 | Validation | Tubular Adenoma | Precancer | MSS |
| LS18_TA2 | Validation | Tubular Adenoma | Precancer | MSI-L |
| LS18_TA3 | Validation | Tubular Adenoma | Precancer | MSI-L |
| LS20_TA1 | Validation | Tubular Adenoma | Precancer | MSI-H |
| LS26_TA1 | Validation | Tubular Adenoma | Precancer | MSI-L |
| LS27_TA1 | Validation | Tubular Adenoma | Precancer | MSI-L |
| LS28_TA1 | Validation | Tubular Adenoma | Precancer | MSI-L |
| LS33_TA1 | Validation | Tubular Adenoma | Precancer | MSI-L |
| LS33_TA2 | Validation | Tubular Adenoma | Precancer | MSI-H |
| LS33_TA3 | Validation | Tubular Adenoma | Precancer | MSI-L |
| LS33_TA4 | Validation | Tubular Adenoma | Precancer | MSI-L |
| LS34_TA1 | Validation | Tubular Adenoma | Precancer | MSI-H |
| LS35_TA1 | Validation | Tubular Adenoma | Precancer | MSI-H |
| LS37_TA1 | Validation | Tubular Adenoma | Precancer | MSI-L |
| LS38_TA1 | Validation | Tubular Adenoma | Precancer | MSI-L |
| LS39_TA1 | Validation | Tubular Adenoma | Precancer | MSI-H |
| LS40_TVA1 | Validation | Tubulovillous Adenoma | Advanced Precancer | MSI-H |

Supplementary Table 4. List of the top 100 most immunogenic predicted MHC-I neoAgs obtained from the computational methods in the
discovery set.

| Mutant | SEQ | | | | | Micro- | Reference | | Altered | Number | Pep- |
|---|---|---|---|---|---|---|---|---|---|---|---|
| Epitope | ID | Gene | Chromo- | | | satellite | MS lengths | Variant | MS Length | deleted | tide |
| Sequence | NO | Name | some | Start | Stop | motif | (repeats) | Type | (repeats) | nucleotides | Length |
| APREGAAATP | 18 | PAWR | chr12 | 80083899 | 80083900 | C | 7 | FS | 6 | −1 | 11 |
| L | | | | | | | | | | | |
| KMMKILMIK | 19 | GOLIM4 | chr3 | 167728580 | 167728581 | A | 7 | FS | 6 | −1 | 9 |
| LSAPEKITLF | 20 | SPINK5 | chr5 | 147499874 | 147499875 | A | 10 | FS | 9 | −1 | 10 |
| LSTEVQSLY | 21 | RAD50 | chr5 | 131931451 | 131931452 | A | 9 | FS | 8 | −1 | 9 |
| KLSSVVPSV | 22 | GPBP1L1 | chr1 | 46120889 | 46120890 | T | 7 | FS | 6 | −1 | 9 |
| YMMDDLELI | 1 | USP9Y | chrY | 14847610 | 14847611 | T | 7 | FS | 6 | −1 | 9 |
| SLWSSMPHG | 23 | P4HB | chr17 | 79803763 | 79803764 | A | 8 | FS | 7 | −1 | 10 |
| V | | | | | | | | | | | |
| TQLARFFPI | 24 | RNF43 | chr17 | 56435160 | 56435161 | G | 7 | FS | 6 | −1 | 9 |
| ALQSDVQPV | 25 | ZFR | chr5 | 32404160 | 32404161 | A | 9 | FS | 8 | −1 | 9 |
| SLINIHHRK | 26 | KMT2C | chr7 | 151874147 | 151874148 | A | 9 | FS | 8 | −1 | 9 |
| RVPAHASTSL | 27 | TCF20 | chr22 | 42564715 | 42564716 | C | 7 | FS | 6 | −1 | 10 |
| IAQPSTSSL | 28 | TCF7L2 | chr10 | 114925316 | 114925317 | A | 9 | FS | 8 | −1 | 9 |
| MLLRLNLRK | 29 | SEC31A | chr4 | 83785564 | 83785565 | A | 9 | FS | 8 | −1 | 9 |
| APSWPDRPL | 30 | NTAN1 | chr16 | 15131989 | 15131990 | A | 7 | FS | 6 | −1 | 9 |
| RLLPYPFHV | 31 | WNK4 | chr17 | 40939869 | 40939870 | G | 7 | FS | 6 | −1 | 9 |
| KMLTALPPA | 32 | WDR59 | chr16 | 74976690 | 74976691 | A | 8 | FS | 7 | −1 | 9 |
| KIKHGLSEK | 33 | OCIAD2 | chr4 | 48894832 | 48894833 | T | 7 | FS | 6 | −1 | 9 |
| YQMDFHPSP | 34 | MFN2 | chr1 | 12052735 | 12052736 | T | 7 | FS | 6 | −1 | 10 |
| V | | | | | | | | | | | |
| SPRPSACQL | 35 | GIPC1 | chr19 | 14593639 | 14593640 | C | 8 | FS | 7 | −1 | 9 |
| QPHVPPSTL | 36 | FLCN | chr17 | 17119708 | 17119709 | C | 8 | FS | 7 | −1 | 9 |
| RLYVPLYSSK | 37 | UBR5 | chr8 | 103289348 | 103289349 | A | 8 | FS | 7 | −1 | 10 |
| LSSPFREQM | 38 | RNF213 | chr17 | 78272285 | 78272288 | CT | 4 | inframe_ | 3 | −2 | 9 |
| | | | | | | | | del | | | |
| IQKSWTATTY | 39 | CTSC | chr11 | 88068107 | 88068108 | T | 6 | FS | 5 | −1 | 10 |
| FLDPDIGGV | 40 | TET2 | chr4 | 106158293 | 106158298 | TGAC | 2 | FS | 1 | −4 | 9 |
| FAMAQIQSL | 41 | SLC4A11 | chr20 | 3215424 | 3215425 | T | 7 | FS | 6 | −1 | 9 |
| RPRLPRHCL | 42 | CIC | chr19 | 42799097 | 42799098 | C | 5 | FS | 4 | −1 | 9 |
| YIMHLWPPI | 43 | VPS13A | chr9 | 79931168 | 79931169 | A | 6 | FS | 5 | −1 | 9 |
| FLATSGIDPV | 44 | WDTC1 | chr1 | 27621107 | 27621108 | G | 8 | FS | 7 | −1 | 10 |
| TLDVELPPV | 45 | PTTG1 | chr5 | 159854836 | 159854837 | C | 6 | FS | 5 | −1 | 9 |
| TLISMPYHV | 46 | SEC63 | chr6 | 108214754 | 108214755 | A | 10 | FS | 9 | −1 | 9 |
| SPMGRKQGG | 47 | IKBKB | chr8 | 42176139 | 42176140 | NA | NA | FS | NA | −1 | 11 |
| TL | | | | | | | | | | | |
| RPKKSGDMT | 48 | BOD1L1 | chr4 | 13610188 | 13610189 | A | 8 | FS | 7 | −1 | 10 |
| L | | | | | | | | | | | |
| NMIQVLMSV | 49 | SMARCAD1 | chr4 | 95173909 | 95173910 | A | 8 | FS | 7 | −1 | 9 |
| SLYGWYQLC | 50 | USP24 | chr1 | 55619561 | 55619562 | A | 7 | FS | 6 | −1 | 10 |
| V | | | | | | | | | | | |
| STMRVAVTP | 51 | RERE | chr1 | 8421827 | 8421828 | A | 5 | FS | 4 | −1 | 10 |
| K | | | | | | | | | | | |
| RNLKNFLLM | 52 | ERBIN | chr5 | 65342358 | 65342359 | T | 5 | FS | 4 | −1 | 10 |
| K | | | | | | | | | | | |
| SLMEQIPHL | 53 | CKAP2 | chr13 | 53049033 | 53049034 | A | 8 | FS | 7 | −1 | |
| RTRGVCSVL | 54 | BTN3A3 | chr6 | 26451946 | 26451947 | T | 6 | FS | 5 | −1 | 10 |
| K | | | | | | | | | | | |
| IMHQYPNFK | 55 | FAM111B | chr11 | 58892376 | 58892377 | A | 10 | FS | 9 | −1 | 9 |
| KTVQAEPLI | 56 | KLHL7 | chr7 | 23163475 | 23163476 | T | 7 | FS | 6 | −1 | 9 |
| KTYMEMHY | 57 | SPECC1 | chr17 | 20108262 | 20108263 | A | 8 | FS | 7 | −1 | 8 |
| RSVLEEMGL | 58 | AP1S1 | chr7 | 100802404 | 100802405 | G | 8 | FS | 7 | −1 | 9 |
| ALQEISFWL | 59 | DYNC1H1 | chr14 | 102445787 | 102445788 | T | 7 | FS | 6 | −1 | 9 |
| RLFSFPAAK | 60 | INF2 | chr14 | 105174184 | 105174185 | C | 7 | FS | 6 | −1 | 9 |
| KSLPSFLTM | 61 | INO80 | chr15 | 41377754 | 41377755 | A | 7 | FS | 6 | −1 | 9 |
| KANRYFSPNF | 62 | PTEN | chr10 | 89720811 | 89720812 | A | 6 | FS | 5 | −1 | 10 |
| SMASIMETI | 63 | C6orf132 | chr6 | 42110042 | 42110043 | G | 5 | FS | 4 | −1 | 9 |
| TQGARSSAAF | 64 | C6orf132 | chr6 | 42074305 | 42074306 | C | 7 | FS | 6 | −1 | 10 |
| FLRLDDLFKL | 65 | PLXNA3 | chrX | 153688564 | 153688565 | G | 8 | FS | 7 | −1 | 10 |
| SLINLTWTA | 66 | CLCA1 | chr1 | 86961263 | 86961264 | A | 4 | FS | 3 | −1 | 9 |
| MMIYFDMEV | 67 | ANO10 | chr3 | 43647212 | 43647213 | A | 9 | FS | 8 | −1 | 9 |
| LLKETKFITY | 68 | TCERG1 | chr5 | 145887464 | 145887465 | A | 8 | FS | 7 | −1 | 10 |
| KSFHGLDFGF | 69 | CEP164 | chr11 | 117234200 | 117234201 | G | 6 | FS | 5 | −1 | 10 |
| RATFLLALW | 70 | MSH3 | chr5 | 79970914 | 79970915 | A | 8 | FS | 7 | −1 | 9 |
| IMMSWMPPL | 71 | PLEKHA6 | chr1 | 204228410 | 204228411 | G | 6 | FS | 5 | −1 | 9 |
| KTHPCTMLL | 72 | ANKIB1 | chr7 | 91936913 | 91936914 | A | 8 | FS | 7 | −1 | 9 |
| APLFRASIL | 73 | CNTROB | chr17 | 7849144 | 7849145 | NA | NA | FS | NA | −1 | 9 |
| WLWENHEKL | 74 | MBD4 | chr3 | 129155547 | 129155548 | A | 10 | FS | 9 | −1 | 9 |
| RRWECSHRL | 75 | MUC5B | chr11 | 1250517 | 1250518 | C | 6 | FS | 5 | −1 | 9 |
| SAFSSLLPL | 76 | RNF25 | chr2 | 219529513 | 219529514 | G | 7 | FS | 6 | −1 | 9 |
| FMQFSLFSV | 77 | SMC3 | chr10 | 112333493 | 112333494 | T | 7 | FS | 6 | −1 | 9 |
| WMIVTVLPV | 78 | POLR2A | chr17 | 7388097 | 7388098 | C | 7 | FS | 6 | −1 | 9 |
| FAFDSPHHY | 79 | APC | chr5 | 112175211 | 112175216 | AAAGA | 2 | FS | 1 | −4 | 9 |
| GRLRVGLRLL | 80 | SCRIB | chr8 | 144886851 | 144886852 | C | 6 | FS | 5 | −1 | 10 |
| FLLTTLLGV | 81 | IL6ST | chr5 | 55247868 | 55247869 | A | 7 | FS | 6 | −1 | 9 |
| KRAARLVLR | 82 | BCORL1 | chrX | 129149049 | 129149050 | A | 5 | FS | 4 | −1 | 9 |
| VLSVRLPTRK | 83 | CPSF1 | chr8 | 145625008 | 145625009 | C | 5 | FS | 4 | −1 | 10 |
| RMKHFIYFK | 84 | TRPM7 | chr15 | 50925139 | 50925140 | A | 7 | FS | 6 | −1 | 9 |
| HRLRSLPRPL | 85 | KMT2D | chr12 | 49445525 | 49445526 | C | 7 | FS | 6 | −1 | 10 |
| FMDQEFLSFV | 86 | SLC44A3 | chr1 | 95357931 | 95357932 | T | 7 | FS | 6 | −1 | 10 |
| LLDDSNFKV | 87 | FAM179B | chr14 | 45432121 | 45432122 | G | 5 | FS | 4 | −1 | 9 |
| SPPVRSTVCA | 88 | HNF1A | chr12 | 121432114 | 121432115 | G | 3 | FS | 2 | −1 | 11 |
| M | | | | | | | | | | | |
| KADFRTLLK | 89 | TCERG1 | chr5 | 145886730 | 145886731 | NA | NA | FS | NA | −1 | 9 |
| FLAVDTQLL | 90 | GRINA | chr8 | 145065717 | 145065718 | C | 7 | FS | 6 | −1 | 9 |
| RVYDPASPQR | 91 | WDR74 | chr11 | 62603470 | 62603472 | AG | 2 | FS | 1 | −1 | 10 |
| FAFDSPHHY | 79 | APC | chr5 | 112174833 | 112174834 | A | 4 | FS | 3 | −1 | 9 |
| RSLQAHKMA | 92 | BPTF | chr17 | 65944265 | 65944266 | A | 7 | FS | 6 | −1 | 10 |
| W | | | | | | | | | | | |
| FLSPWPSPA | 93 | RGL2 | chr6 | 33263964 | 33263965 | G | 8 | FS | 7 | −1 | 9 |
| KRQKLICQM | 94 | SEC16A | chr9 | 139345822 | 139345823 | C | 7 | FS | 6 | −1 | 9 |
| SPPLHLCQPL | 95 | CAMTA2 | chr17 | 4875737 | 4875738 | C | 8 | FS | 7 | −1 | 10 |
| FMNSTVFHV | 3 | WDR6 | chr3 | 49051381 | 49051382 | G | 7 | FS | 6 | −1 | 9 |
| MTIYIFCLHY | 96 | UGCG | chr9 | 114695179 | 114695180 | T | 7 | FS | 6 | −1 | 10 |
| RPACTCISM | 97 | XYLT2 | chr17 | 48433966 | 48433967 | C | 7 | FS | 6 | −1 | 9 |
| LLLGCLCFI | 98 | CHPT1 | chr12 | 102108337 | 102108338 | T | 8 | FS | 7 | −1 | 9 |
| RPENSQINSSL | 99 | TRIM26 | chr6 | 30157253 | 30157254 | A | 8 | FS | 7 | −1 | 11 |
| SLMMIVLTI | 100 | ZMYM2 | chr13 | 20638676 | 20638677 | A | 8 | FS | 7 | −1 | 9 |
| NMMCQHTMI | 101 | AKAP9 | chr7 | 91603084 | 91603085 | A | 8 | FS | 7 | −1 | 9 |
| YLTKWPKFFL | 102 | XPOT | chr12 | 64812754 | 64812755 | T | 9 | FS | 8 | −1 | 10 |
| YSYPSSLSVF | 103 | GPR160 | chr3 | 169802468 | 169802469 | A | 9 | FS | 8 | −1 | 10 |
| TLWSRLVLA | 104 | RAB3GAP2 | chr1 | 220355681 | 220355682 | T | 7 | FS | 6 | −1 | 9 |
| LTSSQSSWW | 105 | MIDN | chr19 | 1257160 | 1257161 | G | 5 | FS | 4 | −1 | 9 |
| ASLAHSDNF | 106 | CLDN4 | chr7 | 73246062 | 73246063 | G | 7 | FS | 6 | −1 | 9 |
| AMAQVTHPL | 107 | PHGR1 | chr15 | 40648423 | 40648424 | C | 7 | FS | 6 | −1 | 9 |
| KMNKILLPW | 108 | SUZ12 | chr17 | 30293208 | 30293209 | A | 5 | FS | 4 | −1 | 10 |
| K | | | | | | | | | | | |
| QLRCWNTWA | 109 | CASP5 | chr11 | 104878040 | 104878041 | A | 10 | FS | 9 | −1 | 10 |
| K | | | | | | | | | | | |
| SLVTISRFV | 110 | RABGAP1 | chr9 | 125861041 | 125861042 | A | 8 | FS | 7 | −1 | 9 |
| YSDENMMDP | 111 | SRGAP1 | chr12 | 64377820 | 64377821 | A | 7 | FS | 6 | −1 | 10 |
| Y | | | | | | | | | | | |
| FLALNQLPQV | 112 | ALG8 | chr11 | 77832192 | 77832193 | A | 7 | FS | 6 | −1 | 10 |
| KPRPLHAL | 113 | ZNF839 | chr14 | 102802051 | 102802052 | T | 6 | FS | 5 | −1 | 8 |
| SLLSVGNLIG | 114 | BTBD7 | chr14 | 93761192 | 93761193 | A | 8 | FS | 7 | −1 | 11 |
| L | | | | | | | | | | | |

| | | | | | | | | | | | | | Pre- |
|---|---|---|---|---|---|---|---|---|---|---|---|---|---|
| | | | | | | | | | | | | | dicted |
| | | | Immuno- | | | | Binding | Tumor | Binding | | | Eli- | in th |
| SEQ | | | geni | | SEQ | | Affi- | Abun- | Sta- | Sample | Eli- | spot | vali- |
| ID | | | city | Wildtype | ID | HLA | nity | dance | bility | Re- | spot | reac- | dation |
| NO | HGVSc | HGVSp | Score | sequence | NO | Allele | (nM) | (TPM) | (hours) | currence | tested | tive | set |
| 18 | NM_002583.2: | NP_002574.2: | 1 | WKAKRE | 777 | HLA- | 5.3 | 110.81 | 52.82 | 1 | Tested | No | No |
| | c.125del | p.Pro42Argfs | | KMRAKQ | | B*07:02 | | | | | | | |
| | | Ter48 | | NPPGPAP | | | | | | | | | |
| | | | | PGGGSSD | | | | | | | | | |
| | | | | AAGKPPA | | | | | | | | | |
| | | | | GALGTPA | | | | | | | | | |
| 19 | NM_01498.4: | NP_055313.1: | 2 | QYQEEAE | 778 | HLA- | 6.5 | 216.91 | 8.6 | 2 | Tested | No | No |
| | 4c.1891del | p.Arg631Glyfs | | EEVQEDL | | A*03:01 | | | | | | | |
| | | Ter87 | | TEEKKRE | | | | | | | | | |
| | | | | LEHNAEE | | | | | | | | | |
| | | | | TYGENDE | | | | | | | | | |
| | | | | NTDDK | | | | | | | | | |
| 20 | NM_001127698.1: | NP_ | 8 | GNKCTM | 779 | HLA- | 12.2 | 461.65 | 1.06 | 3 | Tested | Yes | yes |
| | c.2468del | 001121170.1: | | CKEKLER | | B*15:17 | | | | | | | |
| | | p.Lys823Argfs | | EAAEKK | | | | | | | | | |
| | | Ter119 | | KKEDEDR | | | | | | | | | |
| | | | | SNTGERS | | | | | | | | | |
| | | | | NTGERSN | | | | | | | | | |
| 21 | NM_005732.3: | NP_005723.2: | 11 | SKLRLAP | 780 | HLA- | 4.4 | 39.05 | 3.03 | 2 | No | N/A | No |
| | c.2165del | p.Lys722Argfs | | DKLKSTE | | B*15:17 | | | | | | | |
| | | Ter14 | | SELKKKE | | | | | | | | | |
| | | | | KRRDEM | | | | | | | | | |
| | | | | LGLVPMR | | | | | | | | | |
| | | | | QSIIDL | | | | | | | | | |
| 22 | NM_021639.4: | NP_067652.1: | 13 | RGEGRFG | 781 | HLA- | 4.8 | 64.4 | 43.8 | 1 | Tested | No | No |
| | c.162del | p.Phe54Leufs | | VSRRRHN | | A*02:01 | | | | | | | |
| | | Ter53 | | SSDGFFN | | | | | | | | | |
| | | | | NGPLRTA | | | | | | | | | |
| | | | | GDSWHQ | | | | | | | | | |
| | | | | PSLFRH | | | | | | | | | |
| 1 | NM_004654.3: | NP_004645.2: | 17 | DLINKFG | 782 | HLA- | 2 | 30.44 | 29.55 | 1 | Tested | No | No |
| | c.729del | p.Phe243Leufs | | TLNGFQI | | A*02:01 | | | | | | | |
| | | Ter6 | | LHDRFFN | | | | | | | | | |
| | | | | GSALNIQI | | | | | | | | | |
| | | | | IAALIKPF | | | | | | | | | |
| | | | | GQC | | | | | | | | | |
| 23 | NM_000918.3: | NP_000909.2: | 19 | PVKVLVG | 783 | HLA- | 9.3 | 368.51 | 8.73 | 2 | Tested | Yes | No |
| | c.1160del | p.Asn387Thrfs | | KNFEDVA | | A*02:01 | | | | | | | |
| | | Ter118 | | FDEKKNV | | | | | | | | | |
| | | | | FVEFYAP | | | | | | | | | |
| | | | | WCGHCK | | | | | | | | | |
| | | | | QLAPIW | | | | | | | | | |
| 24 | NM_017763.4: | NP_060233.3: | 26 | FNLQKSS | 784 | HLA- | 16.7 | 79.41 | 1.66 | 8 | Tested | Yes | Yes |
| | c.1976del | p.Gly659Valfs | | LSARHPQ | | A*02:01 | | | | | | | |
| | | Ter41 | | RKRRGGP | | | | | | | | | |
| | | | | SEPTPGS | | | | | | | | | |
| | | | | RPQDATV | | | | | | | | | |
| | | | | HPACQ | | | | | | | | | |
| 25 | NM_016107.3: | NP_057191.2: | 29 | CAGPQTY | 785 | HLA- | 14 | 51.75 | 7.09 | 2 | No | N/A | No |
| | c.1074del | p.Glu359Lysfs | | KEHLEGQ | | A*02:01 | | | | | | | |
| | | Ter4 | | KHKKKE | | | | | | | | | |
| | | | | AALKASQ | | | | | | | | | |
| | | | | NTSSSNS | | | | | | | | | |
| | | | | STRGTQ | | | | | | | | | |
| 26 | NM_170606.2: | NP_733751.2: | 32 | DLPIDDK | 786 | HLA- | 34.8 | 52.26 | 2.06 | 2 | No | N/A | No |
| | c.8390del | p.Lys2797Argfs | | LDNQCVS | | A*03:01 | | | | | | | |
| | | Ter26 | | VEPKKKE | | | | | | | | | |
| | | | | QENKTLV | | | | | | | | | |
| | | | | LSDKHSP | | | | | | | | | |
| | | | | QKKST | | | | | | | | | |
| 27 | NM_005650.2: | NP_005641.1: | 42 | NFSVRCP | 787 | HLA- | 4.8 | 39.27 | 0.98 | 5 | Tested | Yes | No |
| | c.5826del | p.Leu1943Cysfs | | KHKPPLP | | B*07:02 | | | | | | | |
| | | Ter118 | | CPLPPLQ | | | | | | | | | |
| | | | | NKTAKGS | | | | | | | | | |
| | | | | LSTEQSE | | | | | | | | | |
| | | | | RG | | | | | | | | | |
| 28 | NM_001146274.1: | NP_ | 53 | ALFGLDR | 788 | HLA- | 5.1 | 44.54 | 0.35 | 5 | Tested | No | No |
| | c.1403del | 001139746.1: | | QTLWCK | | C*03:03 | | | | | | | |
| | | p.Lys468Serfs | | PCRRKKK | | | | | | | | | |
| | | Ter23 | | CVRYIQG | | | | | | | | | |
| | | | | EGSCLSP | | | | | | | | | |
| | | | | PSSDGS | | | | | | | | | |
| 29 | NM_001318120.1: | NP_ | 62 | DQLQQA | 789 | HLA- | 14.9 | 24.08 | 1.66 | 3 | Tested | Yes | yes |
| | c.1384del | 001305049.1: | | VQSQGFI | | A*03:01 | | | | | | | |
| | | p.Ile462Leufs | | NYCQKKI | | | | | | | | | |
| | | Ter16 | | DASQTEF | | | | | | | | | |
| | | | | EKNVWS | | | | | | | | | |
| | | | | FLKVNFE | | | | | | | | | |
| 30 | NM_173474.3: | NP_775745.1: | 66 | LAEPPHF | 790 | HLA- | 9.1 | 27.57 | 4.21 | 1 | No | N/A | No |
| | c.831del | p.Lys277Asnfs | | VEHIRST | | B*07:02 | | | | | | | |
| | | Ter83 | | LMFLKK | | | | | | | | | |
| | | | | HPSPAHT | | | | | | | | | |
| | | | | LFSGNKA | | | | | | | | | |
| | | | | LLYKKN | | | | | | | | | |
| 31 | NM_032387.4: | NP_115763.2: | 96 | LSSSGFL | 791 | HLA- | 2.4 | 13.58 | 15.23 | 1 | No | N/A | No |
| | c.1822del | p.Val608Cysfs | | DASDPAL | | A*02:01 | | | | | | | |
| | | Ter53 | | QPPGGVP | | | | | | | | | |
| | | | | SSLAESH | | | | | | | | | |
| | | | | LCLPSAF | | | | | | | | | |
| | | | | ALSIP | | | | | | | | | |
| 32 | NM_030581.3: | NP_085058.3: | 97 | PTVALSA | 792 | HLA- | 7.7 | 39.18 | 13.48 | 2 | No | N/A | No |
| | c.479del | p.Asn160Metfs | | VAGASQ | | A*02:01 | | | | | | | |
| | | Ter28 | | VKWNKK | | | | | | | | | |
| | | | | NANCLAT | | | | | | | | | |
| | | | | SHDGDV | | | | | | | | | |
| | | | | RIWDKRK | | | | | | | | | |
| | | | | P | | | | | | | | | |
| 33 | NM_001014446.1: | NP_ | 102 | GFGLGKV | 793 | HLA- | 31.7 | 155.36 | 3.79 | 2 | No | N/A | No |
| | c.339del | 001014446.1: | | SYIGVCQ | | A*03:01 | | | | | | | |
| | | p.Phe113Leufs | | SKFHFFE | | | | | | | | | |
| | | Ter27 | | DQLRGA | | | | | | | | | |
| | | | | GFGPQHN | | | | | | | | | |
| | | | | RHCLLT | | | | | | | | | |
| 34 | NM_001127660.1: | NP_ | 105 | SKVRGIS | 794 | HLA- | 5.4 | 35.57 | 19.6 | 2 | No | N/A | No |
| | c.306del | 001121132.1: | | EVLARRH | | A*02:01 | | | | | | | |
| | | p.Phe102Leufs | | MKVAFF | | | | | | | | | |
| | | Ter11 | | GRTSNGK | | | | | | | | | |
| | | | | STVINAM | | | | | | | | | |
| | | | | LWDKVL | | | | | | | | | |
| 35 | NM_202470.2: | NP_974199.1: | 106 | EPGPLGG | 795 | HLA- | 5.2 | 43.65 | 26 | 1 | No | N/A | No |
| | c.149del | p.Pro50Leufs | | GGSGGPQ | | B*07:02 | | | | | | | |
| | | Ter48 | | MGLPPPP | | | | | | | | | |
| | | | | PALRPRL | | | | | | | | | |
| | | | | VFHTQLA | | | | | | | | | |
| | | | | HGSPT | | | | | | | | | |
| 36 | NM_144997.5: | NP_659434.2: | 112 | EEAYRCN | 796 | HLA- | 26.9 | 60.55 | 5.88 | 1 | No | N/A | No |
| | c.1285del | p.His429Thrfs | | FLGLSPH | | B*07:02 | | | | | | | |
| | | Ter39 | | VQIPPHV | | | | | | | | | |
| | | | | LSSEFAVI | | | | | | | | | |
| | | | | VEVHAA | | | | | | | | | |
| | | | | ARSTL | | | | | | | | | |
| 37 | NM_015902.5: | NP_056986.2: | 119 | MSYAAN | 797 | HLA- | 6.4 | 61.73 | 10.18 | 4 | Tested | Yes | No |
| | c.6360del | p.Glu2121Lysfs | | LKNVMN | | A*03:01 | | | | | | | |
| | | Ter28 | | MQNRQK | | | | | | | | | |
| | | | | KEGEEQP | | | | | | | | | |
| | | | | VLPEETE | | | | | | | | | |
| | | | | SSKPGPS | | | | | | | | | |
| | | | | A | | | | | | | | | |
| 38 | NM_001256071.2: | NP_ | 143 | HKDAWR | 798 | HLA- | 26.5 | 105.69 | 0.35 | 3 | Tested | No | No |
| | c.2180_2182del | 001243000.2: | | QPEDTW | | B*15:17 | | | | | | | |
| | | p.Phe727del | | AALEGLS | | | | | | | | | |
| | | | | FSPFREQ | | | | | | | | | |
| | | | | MLDTSSL | | | | | | | | | |
| | | | | LQFMREK | | | | | | | | | |
| 39 | NM_001814.4: | NP_0018 | 144 | IIYNQGFE | 799 | HLA- | 4.8 | 77.33 | 2.47 | 1 | Tested | Yes | No |
| | c.315del | 05.3:p.Ph | | IVLNDYK | | B*15:03 | | | | | | | |
| | | e105Leuf | | WFAFFKY | | | | | | | | | |
| | | sTer10 | | KEEGSKV | | | | | | | | | |
| | | | | TTYCNET | | | | | | | | | |
| | | | | MTGW | | | | | | | | | |
| 40 | NM_001127208.2: | NP_ | 148 | LKSQKQV | 800 | HLA- | 5.2 | 51.55 | 2.26 | 2 | No | N/A | No |
| | c.3198_3202del | 001120680.1: | | KVEMSGP | | A*02:01 | | | | | | | |
| | | p.Arg1067 | | VTVLTRQ | | | | | | | | | |
| | | AsnfsTer7 | | TTAAELD | | | | | | | | | |
| | | | | SHTPALE | | | | | | | | | |
| | | | | QQTTS | | | | | | | | | |
| 41 | NM_001174090.1: | NP_ | 153 | DEAFDTA | 801 | HLA- | 2.3 | 25.71 | 0.48 | 2 | No | N/A | No |
| | c.333del | 001167561.1: | | NSSIVSGE | | C*03:03 | | | | | | | |
| | | p.Phe111Leufs | | SIRFFVN | | | | | | | | | |
| | | Ter32 | | VNLEMQ | | | | | | | | | |
| | | | | ATNTENE | | | | | | | | | |
| | | | | ATSGG | | | | | | | | | |
| 42 | NM_001304815.1: | NP_ | 163 | KIREVRQ | 802 | HLA- | 3.2 | 24.77 | 36.62 | 1 | No | N/A | No |
| | c.7313del | 001291744.1: | | KIMQAAT | | B*07:02 | | | | | | | |
| | | p.Pro2438Leufs | | PTEQPPG | | | | | | | | | |
| | | Ter91 | | AEAPLPV | | | | | | | | | |
| | | | | PPPTGTA | | | | | | | | | |
| | | | | AAPAP | | | | | | | | | |
| 43 | NM_033305.2: | NP_150648.2: | 164 | EINVIIKN | 803 | HLA- | 3.9 | 189.72 | 10.07 | 2 | No | N/A | No |
| | c.4715del | p.Asn1572Metfs | | PEIVFVA | | A*02:01 | | | | | | | |
| | | Ter6 | | DMTKND | | | | | | | | | |
| | | | | APALVIT | | | | | | | | | |
| | | | | TQCEICY | | | | | | | | | |
| | | | | KGNLE | | | | | | | | | |
| 44 | NM_001276252.1: | NP_ | 178 | ATYVTFS | 804 | HLA- | 4.4 | 10.01 | 4.97 | 6 | Tested | Yes | No |
| | c.868del | 001263181.1: | | PNGTELL | | A*02:01 | | | | | | | |
| | | p.Glu290Asnfs | | VNMGGE | | | | | | | | | |
| | | Ter8 | | QVYLFDL | | | | | | | | | |
| | | | | TYKQRPY | | | | | | | | | |
| | | | | TFLLPR | | | | | | | | | |
| 45 | NM_001282382.1: | NP_ | 182 | PLMILDE | 805 | HLA- | 10.6 | 124.61 | 4.52 | 1 | No | N/A | No |
| | c.491del | 001269311.1: | | ERELEKL | | A*02:01 | | | | | | | |
| | | p.Pro164Leufs | | FQLGPPS | | | | | | | | | |
| | | Ter4 | | PVKMPSP | | | | | | | | | |
| | | | | PWESNLL | | | | | | | | | |
| | | | | QSPSS | | | | | | | | | |
| 46 | NM_007214.4: | NP_009145.1: | 207 | KSKGPKK | 806 | HLA- | 5.3 | 34.43 | 6.72 | 1 | No | N/A | No |
| | c.1605del | p.Lys535Asnfs | | TAKSKKK | | A*02:01 | | | | | | | |
| | | Ter28 | | KPLKKKP | | | | | | | | | |
| | | | | TPVLLPQ | | | | | | | | | |
| | | | | SKQQKQ | | | | | | | | | |
| | | | | KQANGV | | | | | | | | | |
| 47 | NM_001556.2: | NP_001547.1: | 208 | RNLAFFQ | 807 | HLA- | 10.1 | 62.92 | 10.69 | 1 | No | N/A | No |
| | c.1312del | p.Gln438Argfs | | LRKVWG | | B*07:02 | | | | | | | |
| | | Ter3 | | QVWHSIQ | | | | | | | | | |
| | | | | TLKEDCN | | | | | | | | | |
| | | | | RLQQGQ | | | | | | | | | |
| | | | | RAAMMN | | | | | | | | | |
| | | | | L | | | | | | | | | |
| 48 | NM_148894.2: | NP_683692.2: | 222 | PKAARIK | 808 | HLA- | 5.1 | 45.9 | 15.39 | 1 | No | N/A | No |
| | c.1707del | p.Val570Ter | | EVLKERK | | B*07:02 | | | | | | | |
| | | | | VLEKKV | | | | | | | | | |
| | | | | ALSKKRK | | | | | | | | | |
| | | | | KDSRNVE | | | | | | | | | |
| | | | | ENSKKK | | | | | | | | | |
| 49 | NM_001128430.1: | NP_ | 242 | LKQKFSM | 809 | HLA- | 7.3 | 27.16 | 15.29 | 1 | Tested | No | No |
| | c.1040del | 001121902.1: | | KAQNGF | | A*02:01 | | | | | | | |
| | | p.Asn347Metfs | | NKKRKK | | | | | | | | | |
| | | Ter24 | | NVFNPKR | | | | | | | | | |
| | | | | VVEDSEY | | | | | | | | | |
| | | | | DSGSDVG | | | | | | | | | |
| 50 | NM_015306.2: | NP_0561 | 251 | IIKCIEDIK | 810 | HLA- | 14.5 | 34.61 | 8.59 | 1 | No | N/A | No |
| | c.1841del | 21.2:p.As | | RPGEWSG | | A*02:01 | | | | | | | |
| | | n614Thrf | | LEKNKK | | | | | | | | | |
| | | sTer34 | | DGFKSSQ | | | | | | | | | |
| | | | | LNNPQFV | | | | | | | | | |
| | | | | WVVP | | | | | | | | | |
| 51 | NM_001042681.1: | NP_ | 268 | EKVASDT | 811 | HLA- | 13.9 | 15.48 | 7.07 | 2 | No | N/A | No |
| | c.2011del | 001036146.1: | | EEADRTS | | A*03:01 | | | | | | | |
| | | p.Thr671Argfs | | SKKTKTQ | | | | | | | | | |
| | | Ter159 | | EISRPNSP | | | | | | | | | |
| | | | | SEGEGES | | | | | | | | | |
| | | | | SDSR | | | | | | | | | |
| 52 | NM_001 | NP_0012 | 277 | TANMKA | 812 | HLA- | 10 | 71.74 | 0.93 | 1 | No | N/A | No |
| | 253699.1: | 40628.1:p | | SENLKHI | | A*03:01 | | | | | | | |
| | c.1785del | Phe595L | | VNHDDV | | | | | | | | | |
| | | eufsTer13 | | FEESEELS | | | | | | | | | |
| | | | | SDEEMK | | | | | | | | | |
| | | | | MAEMRP | | | | | | | | | |
| | | | | P | | | | | | | | | |
| 53 | NM_001098525.1: | NP_ | 326 | SCLIKYN | 813 | HLA- | 2.4 | 16.3 | 25.69 | 1 | No | N/A | yes |
| | c.1817del | 001091995.1: | | VSTTPYL | | A*02:01 | | | | | | | |
| | | p.Lys606Argfs | | QSVKKK | | | | | | | | | |
| | | Ter14 | | VQFDGTN | | | | | | | | | |
| | | | | SAFKELK | | | | | | | | | |
| | | | | FLTPVR | | | | | | | | | |
| 54 | NM_006994.4: | NP_008925.1: | 333 | LFKPADV | 814 | HLA- | 23.6 | 101.56 | 9.29 | 1 | No | N/A | No |
| | c.1063del | p.Val355Phefs | | ILDPDTA | | A*03:01 | | | | | | | |
| | | Ter71 | | NAILLVS | | | | | | | | | |
| | | | | EDQRSVQ | | | | | | | | | |
| | | | | RAEEPRD | | | | | | | | | |
| | | | | LPDNP | | | | | | | | | |
| 55 | NM_198947.3: | NP_945185.1: | 334 | SMVDEVS | 815 | HLA- | 19.1 | 10.65 | 2.46 | 1 | No | N/A | No |
| | c.816del | p.Ala273Hisfs | | GKVLEM | | A*03:01 | | | | | | | |
| | | Ter26 | | DISKKKA | | | | | | | | | |
| | | | | LQQKDIH | | | | | | | | | |
| | | | | KKIKQNE | | | | | | | | | |
| | | | | SATDEI | | | | | | | | | |
| 56 | NM_001 | NP_ | 341 | VQERKIP | 816 | HLA- | 9.8 | 13.38 | 0.91 | 2 | No | N/A | No |
| | 031710.2: | 001026880.2: | | AHRVVL | | B*15:17 | | | | | | | |
| | c.207del | p.Phe69Leufs | | AAASHFF | | | | | | | | | |
| | | Ter3 | | NLMFTTN | | | | | | | | | |
| | | | | MLESKSF | | | | | | | | | |
| | | | | EVELKD | | | | | | | | | |
| 57 | NM_001243439.1: | NP_ | 345 | SFGSPTG | 817 | HLA- | 19.8 | 25.32 | 2.51 | 3 | Tested | Yes | No |
| | c.908del | 001230368.1: | | NQMSSDI | | B*15:17 | | | | | | | |
| | | p.Asn303Thrfs | | DEYKKNI | | | | | | | | | |
| | | Ter63 | | HGNALRT | | | | | | | | | |
| | | | | SGSSSSD | | | | | | | | | |
| | | | | VTKAS | | | | | | | | | |
| 58 | NM_001283.3: | NP_001274.1: | 360 | IIFNFEKA | 818 | HLA- | 21.6 | 36.11 | 0.41 | 4 | Tested | No | No |
| | c.364del | p.Asp122Metfs | | YFILDEFL | | B*15:17 | | | | | | | |
| | | Ter11 | | MGGDVQ | | | | | | | | | |
| | | | | DTSKKSV | | | | | | | | | |
| | | | | LKAIEQA | | | | | | | | | |
| | | | | DLLQ | | | | | | | | | |
| 59 | NM_001376.4: | NP_001367.2: | 374 | EDSPYET | 819 | HLA- | 5.2 | 143.82 | 5.22 | 1 | No | N/A | No |
| | c.483del | p.Phe161Leufs | | LHSFISNA | | A*02:01 | | | | | | | |
| | | Ter52 | | VAPFFKS | | | | | | | | | |
| | | | | YIRESGK | | | | | | | | | |
| | | | | ADRDGD | | | | | | | | | |
| | | | | KMAPS | | | | | | | | | |
| 60 | NM_022489.3: | NP_071934.3: | 376 | GWGPPPP | 820 | HLA- | 5 | 109.29 | 35.73 | 1 | No | N/A | No |
| | c.1587del | p.Val530Trpfs | | PPPLLPCT | | A*03:01 | | | | | | | |
| | | Ter28 | | CSPPVAG | | | | | | | | | |
| | | | | GMEEVIV | | | | | | | | | |
| | | | | AQVDHG | | | | | | | | | |
| | | | | LGSAW | | | | | | | | | |
| 61 | NM_017553.1: | NP_060023.1: | 387 | KKFKEEK | 821 | HLA- | 3.5 | 27.11 | 1.89 | 2 | No | N/A | No |
| | c.685del | p.Arg229Aspfs | | KLKAKI | | B*15:17 | | | | | | | |
| | | Ter45 | | KKVKKK | | | | | | | | | |
| | | | | RRRDEEL | | | | | | | | | |
| | | | | SSEESPRR | | | | | | | | | |
| | | | | HHHQTK | | | | | | | | | |
| 62 | NM_001304717.2: | NP_ | 391 | CSIERAD | 822 | HLA- | 17.1 | 66.83 | 0.62 | 3 | Tested | No | No |
| | c.1487del | 001291646.2: | | NDKEYL | | B*15:17 | | | | | | | |
| | | pAsn496Metfs | | VLTLTKN | | | | | | | | | |
| | | Ter21 | | DLDKAN | | | | | | | | | |
| | | | | KDKANR | | | | | | | | | |
| | | | | YFSPNFK | | | | | | | | | |
| | | | | V | | | | | | | | | |
| 63 | NM_001164446.1: | NP_ | 399 | YATNPP | 823 | HLA- | 22.3 | 18.59 | 6.4 | 1 | No | N/A | No |
| | c.140del | 001157918.1: | | WIFTQEA | | A*02:01 | | | | | | | |
| | | p.Gly47Alafs | | PEEGTGG | | | | | | | | | |
| | | Ter15 | | FDGIYYG | | | | | | | | | |
| | | | | DNRFNTV | | | | | | | | | |
| | | | | SESGTA | | | | | | | | | |
| 64 | NM_001164446.1: | NP_ | 407 | FTKTPKS | 824 | HLA- | 14.4 | 9.67 | 5.36 | 1 | No | N/A | No |
| | c.1344del | 001157918.1: | | SSPALKP | | B*15:01 | | | | | | | |
| | | p.Ser449Alafs | | KPNPPSP | | | | | | | | | |
| | | Ter68 | | ENTASSA | | | | | | | | | |
| | | | | PVDWRD | | | | | | | | | |
| | | | | PSQMEK | | | | | | | | | |
| 65 | NM_017514.3: | NP_059984.2: | 414 | MPSVCLL | 825 | HLA- | 4.4 | 66.31 | 0.84 | 1 | No | N/A | No |
| | c.49del | p.Ala17Profs | | LLLFLAV | | A*02:01 | | | | | | | |
| | | Ter12 | | GGALGN | | | | | | | | | |
| | | | | RPFRAFV | | | | | | | | | |
| | | | | VTDTTLT | | | | | | | | | |
| | | | | HLA | | | | | | | | | |
| 66 | NM_001285.3: | NP_001276.2: | 415 | GVYSRYF | 826 | HLA- | 5.3 | 1325.16 | 8.55 | 1 | Tested | No | No |
| | c.2022del | p.Val675Cysfs | | TTYDTNG | | A*02:01 | | | | | | | |
| | | Ter14 | | RYSVKVR | | | | | | | | | |
| | | | | ALGGVN | | | | | | | | | |
| | | | | AARRRVI | | | | | | | | | |
| | | | | PQQSGA | | | | | | | | | |
| 67 | NM_001346464.1: | NP_ | 423 | DVKEETK | 827 | HLA- | 2.9 | 33.46 | 15.45 | 2 | No | N/A | No |
| | c.132del | 001333393.1: | | EWLKNRI | | A*02:01 | | | | | | | |
| | | p.Asp45Metfs | | IAKKKDG | | | | | | | | | |
| | | Ter12 | | GAQLLFR | | | | | | | | | |
| | | | | PLLNKYE | | | | | | | | | |
| | | | | QETLE | | | | | | | | | |
| 68 | NM_006706.3: | NP_006697.2: | 437 | LLDETSAI | 828 | HLA- | 29.3 | 92.56 | 14.38 | 2 | No | N/A | No |
| | c.2947del | p.Ile983Serfs | | TLTSTWK | | B*15:01 | | | | | | | |
| | | Ter41 | | EVKKIIKE | | | | | | | | | |
| | | | | DPRCIKFS | | | | | | | | | |
| | | | | SSDRKKQ | | | | | | | | | |
| | | | | RE | | | | | | | | | |
| 69 | NM_014956.4: | NP_055771.4: | 461 | VHSSSEP | 829 | HLA- | 5.4 | 7.75 | 0.99 | 2 | No | N/A | No |
| | c.749del | p.Gly250Valfs | | LRNLHLD | | B*15:17 | | | | | | | |
| | | Ter9 | | IGALGGD | | | | | | | | | |
| | | | | FEYEESL | | | | | | | | | |
| | | | | RTSQPEE | | | | | | | | | |
| | | | | KKDVS | | | | | | | | | |
| 70 | NM_002439.4: | NP_002430.3: | 477 | STSYLLCI | 830 | HLA- | 5.6 | 35.98 | 1.12 | 2 | No | N/A | No |
| | c.1148del | p.Lys383Argfs | | SENKENV | | B*15:17 | | | | | | | |
| | | Ter32 | | RDKKKG | | | | | | | | | |
| | | | | NIFIGIVG | | | | | | | | | |
| | | | | VQPATGE | | | | | | | | | |
| | | | | VVFD | | | | | | | | | |
| 71 | NM_014935.4: | NP_055750.2: | 527 | AQRKSS | 831 | HLA- | 2.3 | 38.45 | 21.68 | 2 | No | N/A | No |
| | c.982del | p.Val328Tyrfs | | MNQLQQ | | A*02:01 | | | | | | | |
| | | Ter172 | | WVNLRR | | | | | | | | | |
| | | | | GVPPPED | | | | | | | | | |
| | | | | LRSPSRF | | | | | | | | | |
| | | | | YPVSRRV | | | | | | | | | |
| | | | | P | | | | | | | | | |
| 72 | NM_019004.1: | NP_061877.1: | 543 | KLDQGE | 832 | HLA- | 28.9 | 17 | 0.32 | 1 | No | N/A | No |
| | c.437del | p.Asn146Thrfs | | YERAAID | | C*16:01 | | | | | | | |
| | | Ter12 | | AVDNKK | | | | | | | | | |
| | | | | NTPLHYA | | | | | | | | | |
| | | | | AASGMK | | | | | | | | | |
| | | | | ACVELLV | | | | | | | | | |
| | | | | K | | | | | | | | | |
| 73 | NM_001037144.5: | NP_ | 554 | EKEERRV | 833 | HLA- | 11.7 | 39.22 | 8.2 | 1 | No | N/A | No |
| | c.1840del | 001032221.1: | | WTMPPM | | B*07:02 | | | | | | | |
| | | p.Val612Tyrfs | | AVALKPV | | | | | | | | | |
| | | Ter86 | | LQQSREA | | | | | | | | | |
| | | | | RDELPGA | | | | | | | | | |
| | | | | PPVLCS | | | | | | | | | |
| 74 | NM_003925.2: | NP_003916.1: | 562 | ACGETLS | 834 | HLA- | 92.4 | 51.75 | 2.74 | 1 | No | N/A | No |
| | c.939del | p.Glu314Lysfs | | VTSEENS | | A*02:01 | | | | | | | |
| | | Ter4 | | LVKKKER | | | | | | | | | |
| | | | | SLSSGSN | | | | | | | | | |
| | | | | FCSEQKT | | | | | | | | | |
| | | | | SGIIN | | | | | | | | | |
| 75 | NM_002458.2: | NP_002449.2: | 563 | NPQRAQL | 835 | HLA- | 5.2 | 2364.48 | 5.86 | 1 | No | N/A | No |
| | c.1100del | p.Pro367Glnfs | | CEDHCV | | B*27:05 | | | | | | | |
| | | Ter105 | | DGCFCPP | | | | | | | | | |
| | | | | GTVLDDI | | | | | | | | | |
| | | | | THSGCLP | | | | | | | | | |
| | | | | LGQCPC | | | | | | | | | |
| 76 | NM_022453.2: | NP_071898.2: | 585 | SLRQQEE | 836 | HLA- | 20.8 | 11.95 | 0.59 | 2 | No | N/A | No |
| | c.749del | p.Gly250Glufs | | RKRLYQR | | B*15:17 | | | | | | | |
| | | Ter29 | | QQERGGI | | | | | | | | | |
| | | | | IDLEAER | | | | | | | | | |
| | | | | NRYFISL | | | | | | | | | |
| | | | | QQPPA | | | | | | | | | |
| 77 | NM_005445.3: | NP_005436.1: | 593 | SSKHNVI | 837 | HLA- | 2.4 | 54.42 | 8.6 | 1 | No | N/A | No |
| | c.127del | p.Tyr43Metfs | | VGRNGS | | A*02:01 | | | | | | | |
| | | | | GKSNFFY | | | | | | | | | |
| | | Ter69 | | AIQFVLS | | | | | | | | | |
| | | | | DEFSHLR | | | | | | | | | |
| | | | | PEQRLA | | | | | | | | | |
| 78 | NM_000937.4: | NP_000928.1: | 605 | MHGGGP | 838 | HLA- | 9.1 | 104.61 | 8.49 | 1 | No | N/A | No |
| | c.21del | p.Ser8Argfs | | PSGDSAC | | A*02:01 | | | | | | | |
| | | Ter19 | | PLRTIKR | | | | | | | | | |
| | | | | VQFGVLS | | | | | | | | | |
| | | | | P | | | | | | | | | |
| 79 | NM_000038.5: | NP_000029.2: | 616 | NQTTQEA | 839 | HLA- | 3.3 | 5.48 | 3.32 | 1 | No | N/A | No |
| | c.3927_ | p.Glu1309Aspfs | | DSANTLQ | | B*35:01 | | | | | | | |
| | 3931del | Ter4 | | IAEIKEKI | | | | | | | | | |
| | | | | GTRSAED | | | | | | | | | |
| | | | | PVSEVPA | | | | | | | | | |
| | | | | VSQH | | | | | | | | | |
| 80 | NM_182706.4: | NP_874365.3: | 622 | EREAGGP | 840 | HLA- | 10.9 | 26.19 | 0.67 | 1 | No | N/A | No |
| | c.2895del | p.Thr966Profs | | LPPSPLPH | | B*27:05 | | | | | | | |
| | | Ter9 | | SSPPTAA | | | | | | | | | |
| | | | | VATTSITT | | | | | | | | | |
| | | | | ATPGVPG | | | | | | | | | |
| | | | | LPS | | | | | | | | | |
| 81 | NM_002184.3: | NP_0021 | 633 | KAYLKQ | 841 | HLA- | 2.8 | 14.88 | 15.53 | 2 | No | N/A | No |
| | c.1587del | 75.2:p. Va | | APPSKGP | | A*02:01 | | | | | | | |
| | | | | TVRTKKV | | | | | | | | | |
| | | | | GKNEAV | | | | | | | | | |
| | | 1530Ter | | LEWDQLP | | | | | | | | | |
| | | | | VDVQNG | | | | | | | | | |
| | | | | F | | | | | | | | | |
| 82 | NM_001184772.2: | NP_ | 651 | PISIIDQG | 842 | HLA- | 29.4 | 5.74 | 6.82 | 1 | No | N/A | No |
| | c.2306del | 001171701.1: | | EPKGTGA | | B*27:05 | | | | | | | |
| | | p.Lys769Argfs | | TCGKKGS | | | | | | | | | |
| | | Ter14 | | QAGAEG | | | | | | | | | |
| | | | | QPSTVKR | | | | | | | | | |
| | | | | YTPAR | | | | | | | | | |
| 83 | NM_013291.2: | NP_037423.2: | 659 | GSRLGNS | 843 | HLA- | 42.2 | 76.17 | 1.23 | 1 | No | N/A | No |
| | c.1211del | p.Pro404Argfs | | LLLKYTE | | A*03:01 | | | | | | | |
| | | Ter67 | | KLQEPPA | | | | | | | | | |
| | | | | SAVREAA | | | | | | | | | |
| | | | | DKEEPPS | | | | | | | | | |
| | | | | KKKRV | | | | | | | | | |
| 84 | NM_017672.5: | NP_060142.3: | 663 | EGGNLPD | 844 | HLA- | 6.1 | 45.7 | 11.8 | 1 | No | N/A | No |
| | c.1057del | p.Thr353Hisfs | | AAEPDIIS | | A*03:01 | | | | | | | |
| | | Ter16 | | TIKKTFN | | | | | | | | | |
| | | | | FGQNEAL | | | | | | | | | |
| | | | | HLFQTLM | | | | | | | | | |
| | | | | ECMK | | | | | | | | | |
| 85 | NM_003482.3: | NP_003473.3: | 669 | PPPEDSP | 845 | HLA- | 24.6 | 38.47 | 0.91 | 1 | No | N/A | No |
| | c.1940del | p.Pro647Hisfs | | MSPPPEE | | B*27:05 | | | | | | | |
| | | Ter283 | | SPMSPPP | | | | | | | | | |
| | | | | EVSRLSP | | | | | | | | | |
| | | | | LPVVSRL | | | | | | | | | |
| | | | | SPPPE | | | | | | | | | |
| 86 | NM_001114106.2: | NP_ | 670 | FNYNRAF | 846 | HLA- | 3.5 | 39.8 | 9.32 | 2 | No | N/A | No |
| | c.1722del | 001107578.1: | | QVWAVP | | A*02:01 | | | | | | | |
| | | p.Phe574Leufs | | LLLVAFF | | | | | | | | | |
| | | Ter4 | | AYLVAHS | | | | | | | | | |
| | | | | FLSVFET | | | | | | | | | |
| | | | | VLDALF | | | | | | | | | |
| 87 | NM_001308120.1: | NP_ | 678 | SDEKRLC | 847 | HLA- | 5.8 | 15.62 | 4.55 | 2 | No | N/A | No |
| | c.502del | 001295049.1: | | LQLLSDV | | A*02:01 | | | | | | | |
| | | p.Glu168Argfs | | LRGQGEA | | | | | | | | | |
| | | Ter11 | | GQLEEAF | | | | | | | | | |
| | | | | SLALLPQ | | | | | | | | | |
| | | | | LVVSL | | | | | | | | | |
| 88 | NM_001306179.1: | NP_ | 686 | RKEEAFR | 848 | HLA- | 21.3 | 27.56 | 3.48 | 2 | No | N/A | No |
| | c.864del | 001293108.1: | | HKLAMD | | B*07:02 | | | | | | | |
| | | p.Pro291Glnfs | | TYSGPPP | | | | | | | | | |
| | | Ter51 | | GPGPGPA | | | | | | | | | |
| | | | | LPAHSSP | | | | | | | | | |
| | | | | GLPPPA | | | | | | | | | |
| 89 | NM_006706.3: | NP_006697.2: | 701 | REEKEKL | 849 | HLA- | 72.1 | 83.94 | 2.07 | 1 | No | N/A | No |
| | c.2871del | p.Arg958Glufs | | FNEHIEA | | A*03:01 | | | | | | | |
| | | Ter16 | | LTKKKRE | | | | | | | | | |
| | | | | HFRQLLD | | | | | | | | | |
| | | | | ETSAITLT | | | | | | | | | |
| | | | | STWK | | | | | | | | | |
| 90 | NM_001009184.1: | NP_ | 776 | PYPQGGY | 850 | HLA- | 63 | 64.4 | 3.23 | 2 | No | N/A | No |
| | c.333del | 001009184.1: | | PQGPYPQ | | A*02:01 | | | | | | | |
| | | p.Asn112Thrfs | | SPFPPNPY | | | | | | | | | |
| | | Ter56 | | GQPQVFP | | | | | | | | | |
| | | | | GQDPDSP | | | | | | | | | |
| | | | | QHGN | | | | | | | | | |
| 91 | NM_018093.3: | NP_060563.2: | 835 | RGLAQA | 851 | HLA- | 40 | 18.78 | 4.22 | 2 | No | N/A | No |
| | c.330_331del | p.Arg110Serfs | | DGTLITC | | A*03:01 | | | | | | | |
| | | Ter4 | | VDSGILR | | | | | | | | | |
| | | | | VWHDKD | | | | | | | | | |
| | | | | KDTSSDP | | | | | | | | | |
| | | | | LLELRVG | | | | | | | | | |
| 79 | NM_000038.5: | NP_000029.2: | 853 | SIKYNEE | 852 | HLA- | 23.4 | 36.82 | 0.31 | 1 | No | N/A | No |
| | c.3546del | p.Lys1182Asnfs | | KRHVDQ | | C*16:01 | | | | | | | |
| | | Ter83 | | PIDYSLK | | | | | | | | | |
| | | | | YATDIPSS | | | | | | | | | |
| | | | | QKQSFSF | | | | | | | | | |
| | | | | SKSSS | | | | | | | | | |
| 92 | NM_182641.3: | NP_872579.2: | 882 | QVMKYIL | 853 | HLA- | 6.6 | 39.3 | 1.3 | 2 | No | N/A | No |
| | c.7776del | p.Lys2592Asnfs | | DKIDKEE | | B*15:17 | | | | | | | |
| | | Ter36 | | KQAAKK | | | | | | | | | |
| | | | | RKREESV | | | | | | | | | |
| | | | | EQKRSKQ | | | | | | | | | |
| | | | | NATKLS | | | | | | | | | |
| 93 | NM_004761.4: | NP_004752.1: | 922 | RLESFLL | 854 | HLA- | 5.6 | 20.53 | 4.54 | 1 | No | N/A | No |
| | c.608del | p.Gly203Alafs | | QTGYAA | | A*02:01 | | | | | | | |
| | | Ter49 | | GKGVGG | | | | | | | | | |
| | | | | GSADLIR | | | | | | | | | |
| | | | | NLRSRVD | | | | | | | | | |
| | | | | PQAPDLP | | | | | | | | | |
| 94 | NM_014866.1: | NP_055681.1: | 933 | DGKFANL | 855 | HLA- | 19.7 | 23.93 | 1.68 | 1 | No | N/A | No |
| | c.6197del | p.Pro2066Glnfs | | TPSRTVP | | B*27:05 | | | | | | | |
| | | Ter76 | | DSEAPPG | | | | | | | | | |
| | | | | WDRADS | | | | | | | | | |
| | | | | GPTQPPL | | | | | | | | | |
| | | | | SLSPAP | | | | | | | | | |
| 95 | NM_001171167.1: | NP_ | 935 | DGTFSVT | 856 | HLA- | 76 | 58.89 | 2.29 | 3 | Tested | No | yes |
| | c.2666del | 001164638.1: | | SAYSSAP | | B*07:02 | | | | | | | |
| | | p.Pro889Leufs | | DGSPPPA | | | | | | | | | |
| | | Ter9 | | PLPASEM | | | | | | | | | |
| | | | | TMEDMA | | | | | | | | | |
| | | | | PGQLSS | | | | | | | | | |
| 3 | NM_018031.3: | NP_060501.3: | 953 | GGPQDPQ | 857 | HLA- | 2 | 30.6 | 29.16 | 2 | Tested | No | No |
| | c.2511del | p.Arg838Glyfs | | PGLTAHV | | A*02:01 | | | | | | | |
| | | Ter33 | | VSAGGR | | | | | | | | | |
| | | | | AEMHCFS | | | | | | | | | |
| | | | | IMVTPDP | | | | | | | | | |
| | | | | STPSRL | | | | | | | | | |
| 96 | NM_003358.1: | NP_003349.1: | 958 | KLDYAV | 858 | HLA- | 39.5 | 32.69 | 3.2 | 2 | No | N/A | No |
| | c.1094del | p.Leu365Cysfs | | AWFIRES | | B*15:17 | | | | | | | |
| | | Ter9 | | MTIYIFLS | | | | | | | | | |
| | | | | ALWDPTI | | | | | | | | | |
| | | | | SWRTGR | | | | | | | | | |
| | | | | YRLRCG | | | | | | | | | |
| 97 | NM_022167.2: | NP_071450.2: | 969 | VNQEVLE | 859 | HLA- | 7.6 | 10.17 | 19.8 | 3 | Tested | No | yes |
| | c.1584del | p.Gly529Alafs | | ILDFHLY | | B*07:02 | | | | | | | |
| | | Ter78 | | GSYPPGT | | | | | | | | | |
| | | | | PALKAY | | | | | | | | | |
| | | | | WENTYD | | | | | | | | | |
| | | | | AADGPSG | | | | | | | | | |
| 98 | NM_020244.2: | NP_064629.2: | 977 | MAVGASI | 860 | HLA- | 11.2 | 33.89 | 15.99 | 1 1 | No | N/A | No |
| | c.485del | p.Phe162Serfs | | AARLGTY | | A*02:01 | | | | | | | |
| | | Ter23 | | PDWFFFC | | | | | | | | | |
| | | | | SFIGMFV | | | | | | | | | |
| | | | | FYCAHW | | | | | | | | | |
| | | | | QTYVSG | | | | | | | | | |
| 99 | NM_001242783.1: | NP_ | 993 | RYPRKKF | 861 | HLA- | 23.4 | 25.39 | 4.31 | 1 | No | N/A | No |
| | c.845del | 001229712.1: | | WVGKPIA | | B*07:02 | | | | | | | |
| | | p.Lys282Argfs | | RVVKKK | | | | | | | | | |
| | | Ter16 | | TGEFSDK | | | | | | | | | |
| | | | | LLSLQRG | | | | | | | | | |
| | | | | LREFQG | | | | | | | | | |
| 100 | NM_003453.4: | NP_003444.1: | 1003 | LPPVFGE | 862 | HLA- | 35.4 | 32.92 | 13.15 | 1 | No | N/A | No |
| | c.3131del | p.Lys1044Argfs | | EYEEQPR | | A*02:01 | | | | | | | |
| | | Ter33 | | PRSKKKG | | | | | | | | | |
| | | | | AKRKAV | | | | | | | | | |
| | | | | SGYQSHD | | | | | | | | | |
| | | | | DSSDNS | | | | | | | | | |
| 101 | NM_005751.4: | NP_005742.4: | 1004 | FRQRKAQ | 863 | HLA- | 139.6 | 90.94 | 2.06 | 2 | No | N/A | No |
| | c.116del | p.Lys39Argfs | | SDGQSPS | | A*02:01 | | | | | | | |
| | | Ter17 | | KKQKKK | | | | | | | | | |
| | | | | RKTSSSK | | | | | | | | | |
| | | | | HDVSAH | | | | | | | | | |
| | | | | HDLNIDQ | | | | | | | | | |
| 102 | NM_007235.4: | NP_009166.2: | 1007 | QVFALLF | 864 | HLA- | 12.5 | 24.91 | 4.44 | 1 | No | N/A | No |
| | c.378del | p.Phe126Leufs | | VTEYLTK | | A*02:01 | | | | | | | |
| | | Ter6 | | WPKFFFD | | | | | | | | | |
| | | | | ILSVVDL | | | | | | | | | |
| | | | | NPRGVDL | | | | | | | | | |
| | | | | YLRIL | | | | | | | | | |
| 103 | NM_014373.2: | NP_055188.1: | 1013 | TILYFPFS | 865 | HLA- | 2.6 | 9.55 | 3.5 | 2 | No | N/A | No |
| | c.715del | p.Ile239Tyrfs | | SHSSYTV | | B*15:17 | | | | | | | |
| | | Ter22 | | RSKKIFLS | | | | | | | | | |
| | | | | KLIVCFLS | | | | | | | | | |
| | | | | TWLPFVL | | | | | | | | | |
| | | | | LQ | | | | | | | | | |
| 104 | NM_012414.3: | NP_036546.2: | 1038 | LNIKKISE | 866 | HLA- | 19.6 | 23.15 | 6.01 | 1 | No | N/A | No |
| | c.2227del | p.Trp743Glyfs | | EEYVALG | | A*02:01 | | | | | | | |
| | | Ter32 | | SFFFWKC | | | | | | | | | |
| | | | | LHGESST | | | | | | | | | |
| | | | | EDMCHT | | | | | | | | | |
| | | | | LESAG | | | | | | | | | |
| 105 | NM_177401.4: | NP_796375.3: | 1064 | PYHWSPS | 867 | HLA- | 7.8 | 17.05 | 0.55 | 2 | No | N/A | No |
| | c.1301del | p.Gly434Alafs | | RKAGRSD | | B*15:17 | | | | | | | |
| | | Ter91 | | SSSSGGG | | | | | | | | | |
| | | | | GSPSEAS | | | | | | | | | |
| | | | | GLGLDFE | | | | | | | | | |
| | | | | DSVWK | | | | | | | | | |
| 106 | NM_001305.3: | NP_001296.1: | 1072 | GASLYVG | 868 | HLA- | 20.5 | 19.67 | 0.41 | 3 | Tested | No | No |
| | c.537del | p.Leu180Cysfs | | WAASGL | | B*15:17 | | | | | | | |
| | | Ter115 | | LLLGGGL | | | | | | | | | |
| | | | | LCCNCPP | | | | | | | | | |
| | | | | RTDKPYS | | | | | | | | | |
| | | | | AKYSAA | | | | | | | | | |
| 107 | NM_001145643.1: | NP_ | 1118 | PGPCGPP | 869 | HLA- | 10.4 | 44.01 | 11.11 | 1 | No | N/A | yes |
| | c.175del | 001139115.1: | | PGHGPGP | | A*02:01 | | | | | | | |
| | | p.His59Thrfs | | CGPPPHH | | | | | | | | | |
| | | Ter49 | | GPGPCGP | | | | | | | | | |
| | | | | PPGHGPG | | | | | | | | | |
| | | | | HPPPG | | | | | | | | | |
| 108 | NM_015355.2: | NP_056170.2: | 1127 | ESHSLSA | 870 | HLA- | 10.9 | 22.52 | 3.74 | 1 | No | N/A | No |
| | c.503del | p.Asn168Metfs | | HLQLTFT | | A*03:01 | | | | | | | |
| | | Ter22 | | GFFHKND | | | | | | | | | |
| | | | | KPSPNSE | | | | | | | | | |
| | | | | NEQNSVT | | | | | | | | | |
| | | | | LEVLL | | | | | | | | | |
| 109 | NM_001136112.1: | NP_ | 1144 | VPNTDQK | 871 | HLA- | 74.7 | 167.91 | 1.6 | 2 | No | N/A | No |
| | c.241del | 001129584.1: | | STSVKKD | | A*03:01 | | | | | | | |
| | | p.Thr81Glnfs | | NHKKKT | | | | | | | | | |
| | | Ter26 | | VKMLEY | | | | | | | | | |
| | | | | LGKDVL | | | | | | | | | |
| | | | | HGVFNYL | | | | | | | | | |
| | | | | A | | | | | | | | | |
| 110 | NM_012197.3: | NP_036329.3: | 1151 | QLKEMC | 872 | HLA- | 64.2 | 39.43 | 2.55 | 2 | No | N/A | No |
| | c.2789del | p.Asn930Thrfs | | RRELDKA | | A*02:01 | | | | | | | |
| | | Ter15 | | ESEIKKN | | | | | | | | | |
| | | | | SSIIGDYK | | | | | | | | | |
| | | | | QICSQLSE | | | | | | | | | |
| | | | | RLEK | | | | | | | | | |
| 111 | NM_020762.2: | NP_065813.1: | 1199 | QTEMRV | 873 | HLA- | 5.7 | 6.72 | 2.56 | 2 | No | N/A | No |
| | c.168del | p.Ala57Leufs | | QLLQDLQ | | A*01:01 | | | | | | | |
| | | Ter11 | | DFFRKKA | | | | | | | | | |
| | | | | EIETEYSR | | | | | | | | | |
| | | | | NLEKLAE | | | | | | | | | |
| | | | | RFMAK | | | | | | | | | |
| 112 | NM_024079.4: | NP_076984.2: | 1212 | DVLFVYA | 874 | HLA- | 4.5 | 34.91 | 11.76 | 1 | Tested | No | No |
| | c.396del | p.Val133Trpfs | | VRECCKC | | A*02:01 | | | | | | | |
| | | Ter24 | | IDGKKVG | | | | | | | | | |
| | | | | KELTEKP | | | | | | | | | |
| | | | | KFILSVLL | | | | | | | | | |
| | | | | LWNF | | | | | | | | | |
| 113 | NM_018335.4: | NP_060805.3: | 1219 | TVYEFLL | 875 | HLA- | 4.5 | 34.91 | 45.92 | 1 | No | N/A | No |
| | c.1541del | p.Phe514Serfs | | MKVEKD | | B*07:02 | | | | | | | |
| | | Ter36 | | HLAKPFF | | | | | | | | | |
| | | | | PAIYKEF | | | | | | | | | |
| | | | | EELHKM | | | | | | | | | |
| | | | | VKKMCQ | | | | | | | | | |
| | | | | D | | | | | | | | | |
| 114 | NM_001002860.2: | NP_ | 1247 | CESKLYS | 876 | HLA- | 59.3 | 30.99 | 2.22 | 2 | No | N/A | No |
| | c.173del | 001002860.2: | | LDHGHE | | A*02:01 | | | | | | | |
| | | p.Lys58Argfs | | KPQDKK | | | | | | | | | |
| | | Ter44 | | KRTSGLA | | | | | | | | | |
| | | | | TLKKKFI | | | | | | | | | |
| | | | | KRRKSNR | | | | | | | | | |

Supplementary Table 5. List of the Top 100 most recurrent predicted MHC-I neoAgs, with higher immunogenicity, obtained from the
computational methods in the discovery set.

| Mutant | SEQ | | | | | Micro- | Reference | | Altered | Number | Pep- |
|---|---|---|---|---|---|---|---|---|---|---|---|
| Epitope | ID | Gene | Chromo- | | | satellite | MS lengths | Variant | MS Length | deleted | tide |
| Sequence | NO | Name | some | Start | Stop | motif | (repeats) | Type | (repeats) | nucleotides | Length |
| ATQLARFFPI | 24 | RNF43 | chr17 | 56435160 | 56435161 | G | 7 | FS | 6 | −1 | 9 |
| SQVWTAATL | 115 | DOCK3 | chr3 | 51417603 | 51417604 | C | 7 | FS | 6 | −1 | 10 |
| R | | | | | | | | | | | |
| GMVPLIIPV | 116 | ELMSAN1 | chr14 | 74205772 | 74205773 | C | 7 | FS | 6 | −1 | 9 |
| FLATSGIDPV | 44 | WDTC1 | chr1 | 27621107 | 27621108 | G | 8 | FS | 7 | −1 | 10 |
| TPQDSRQVL | 117 | BMPR2 | chr2 | 203420129 | 203420130 | A | 7 | FS | 6 | −1 | 9 |
| RAWRRFPLL | 118 | MICAL3 | chr22 | 18300931 | 18300932 | C | 7 | FS | 6 | −1 | 9 |
| VGMRETTGL | 119 | ASTE1 | chr3 | 130733046 | 130733047 | A | 11 | FS | 10 | −1 | 9 |
| TSSPRTMSW | 120 | MFRP | chr11 | 119213687 | 119213688 | C | 7 | FS | 6 | −1 | 9 |
| SWMGGLHSF | 121 | OR4M1 | chr14 | 20248930 | 20248930 | G | 6 | FS | 7 | 1 | 10 |
| Y | | | | | | | | | | | |
| RVPAHASTSL | 27 | TCF20 | chr22 | 42564715 | 42564716 | C | 7 | FS | 6 | −1 | 10 |
| IAQPSTSSL | 28 | TCF7L2 | chr10 | 114925316 | 114925317 | A | 9 | FS | 8 | −1 | 9 |
| SQKNITPAI | 122 | TGFBR2 | chr3 | 30691871 | 30691872 | A | 10 | FS | 9 | −1 | 9 |
| KADQSESSL | 123 | CHD3 | chr17 | 7798764 | 7798765 | C | 7 | FS | 6 | −1 | 9 |
| LTHPAHQPL | 124 | ARID1A | chr1 | 27105930 | 27105931 | G | 7 | FS | 6 | −1 | 9 |
| RTLLVTCILY | 125 | LARP4B | chr10 | 890938 | 890939 | A | 7 | FS | 6 | −1 | 10 |
| RSAFPSRSL | 126 | MARCKS | chr6 | 114181209 | 114181210 | A | 11 | FS | 10 | −1 | 9 |
| VVHKKRGLF | 127 | ACVR2A | chr2 | 148683685 | 148683686 | A | 8 | FS | 7 | −1 | 9 |
| LSWRGASFI | 128 | OR4M2 | chr15 | 22369023 | 22369024 | G | 7 | FS | 6 | −1 | 9 |
| QSYNTVTRQ | 129 | KLHL42 | chr12 | 27950768 | 27950769 | G | 7 | FS | 6 | −1 | 10 |
| W | | | | | | | | | | | |
| WTGSCRQGW | 130 | B4GALNT4 | chr11 | 380920 | 380921 | G | 7 | FS | 6 | −1 | 9 |
| RLYVPLYSSK | 37 | UBR5 | chr8 | 103289348 | 103289349 | A | 8 | FS | 7 | −1 | 10 |
| RSVLEEMGL | 58 | AP1S1 | chr7 | 100802404 | 100802405 | G | 8 | FS | 7 | −1 | 9 |
| KRAFIHTPR | 131 | STAMBPL1 | chr10 | 90682145 | 90682146 | A | 8 | FS | 7 | −1 | 9 |
| LKLCSKVSF | 132 | CASP5 | chr11 | 104879686 | 104879687 | A | 10 | FS | 9 | −1 | 9 |
| KVDTHHLQV | 133 | PRDM2 | chr1 | 14108748 | 14108749 | A | 9 | FS | 8 | −1 | 9 |
| SPSRSTTAPV | 134 | CELSR1 | chr22 | 46931226 | 46931227 | G | 6 | FS | 5 | −1 | 10 |
| FLQEVFQA | 5 | MARCKS | chr6 | 114181209 | 114181211 | A | 11 | FS | 9 | −1 | 8 |
| CRREYRVTM | 135 | COBLL1 | chr2 | 165551295 | 165551296 | T | 9 | FS | 8 | −1 | 9 |
| WSWCGTSQT | 136 | BCORL1 | chrX | 129190010 | 129190011 | C | 7 | FS | 6 | −1 | 10 |
| Y | | | | | | | | | | | |
| KPLWRKSPL | 137 | TMEM94 | chr17 | 7349062 | 73491063 | C | 7 | FS | 6 | −1 | 9 |
| RLSCAPPPI | 138 | SLC23A2 | chr20 | 4850568 | 4850569 | C | 9 | FS | 8 | −1 | 9 |
| LSSWFSPTV | 139 | TMEM132D | chr12 | 130184704 | 130184705 | C | 7 | FS | 6 | −1 | 9 |
| MSSIWGTMF | 140 | SLC22A9 | chr11 | 63149670 | 63149671 | A | 11 | FS | 10 | −1 | 9 |
| RTRSAWGDW | 141 | MYCN | chr2 | 16082313 | 16082314 | C | 7 | FS | 6 | −1 | 9 |
| RTIMGWTLD | 142 | BAX | chr19 | 49458970 | 49458971 | G | 8 | FS | 7 | −1 | 10 |
| F | | | | | | | | | | | |
| ASRPGSFTF | 143 | TRIO | chr5 | 14487780 | 14487781 | C | 7 | FS | 6 | −1 | 9 |
| KSLEGNLETF | 144 | NES | chr1 | 156642803 | 156642804 | C | 7 | FS | 6 | −1 | 10 |
| LALPCRSVW | 145 | LIPE | chr19 | 42905972 | 42905973 | G | 6 | FS | 5 | −1 | 9 |
| VLEFSSDRKK | 146 | ATP8A2 | chr13 | 26151212 | 26151212 | A | 5 | FS | 10 | 4 | 10 |
| KAFLPERKCF | 147 | CCDC15 | chr1 | 124845048 | 124845049 | A | 8 | FS | 7 | −1 | 10 |
| TNTMGGVQG | 148 | DAPK1 | chr9 | 90321801 | 90321801 | G | 7 | FS | 8 | 1 | 10 |
| K | | | | | | | | | | | |
| GSHNIKKAW | 149 | TMEM60 | chr7 | 77423459 | 77423460 | A | 9 | FS | 8 | −1 | 10 |
| Y | | | | | | | | | | | |
| LSAPEKITLF | 20 | SPINK5 | chr5 | 147499874 | 147499875 | A | 10 | FS | 9 | −1 | 10 |
| MLLRLNLRK | 29 | SEC31A | chr4 | 83785564 | 83785565 | A | 9 | FS | 8 | −1 | 9 |
| LSSPFREQM | 38 | RNF213 | chr17 | 78272285 | 78272288 | CT | 4 | inframe_ | 3 | −2 | 9 |
| | | | | | | | | del | | | |
| KTYMEMHY | 57 | SPECC1 | chr17 | 20108262 | 20108263 | A | 8 | FS | 7 | −1 | 8 |
| KANRYFSPNF | 62 | PTEN | chr10 | 89720811 | 89720812 | A | 6 | FS | 5 | −1 | 10 |
| SPPLHLCQPL | 95 | CAMTA2 | chr17 | 4875737 | 4875738 | C | 8 | FS | 7 | −1 | 10 |
| RPACTCISM | 97 | XYLT2 | chr17 | 48433966 | 48433967 | C | 7 | FS | 6 | −1 | 9 |
| ASLAHSDNF | 106 | CLDN4 | chr7 | 73246062 | 73246063 | G | 6 | FS | 5 | −1 | 9 |
| AMAENILAA | 150 | NOL4L | chr20 | 31041555 | 31041556 | C | 8 | FS | 7 | −1 | 9 |
| YLGTPTWNC | 151 | FAM83D | chr20 | 37580942 | 37580943 | CA | 3 | FS | 2 | −1 | 9 |
| RRPLRSWTPR | 152 | CNKSR1 | chr1 | 26510310 | 26510311 | C | 7 | FS | 6 | −1 | 10 |
| RAWRAGMPL | 153 | COL9A2 | chr1 | 40769746 | 40769747 | C | 4 | FS | 3 | −1 | 9 |
| HSWRFCTHIR | 154 | SIN3A | chr15 | 75703909 | 75703910 | NA | NA | FS | NA | −1 | 10 |
| MPCFTTALLL | 155 | PGD | chr1 | 10479542 | 10479544 | CT | 4 | FS | 3 | −1 | 10 |
| QTIEERLTW | 156 | CRIM1 | chr2 | 36764627 | 36764628 | C | 6 | FS | 5 | −1 | 9 |
| VMANVLTLN | 157 | AASDH | chr4 | 57220268 | 57220269 | T | 10 | FS | 9 | −1 | 10 |
| L | | | | | | | | | | | |
| VLEDTLLKI | 158 | CCDC186 | chr10 | 115885657 | 115885658 | A | 6 | FS | 5 | −1 | 9 |
| KLYEAVPQL | 159 | CDC7 | chr1 | 91967356 | 91967357 | A | 9 | FS | 8 | −1 | 9 |
| AGIGWGASY | 160 | HOXA11 | chr7 | 27222461 | 27222462 | A | 9 | FS | 8 | −1 | 9 |
| RMASTSCAA | 161 | ZFP36L2 | chr2 | 43452622 | 43452623 | G | 6 | FS | 5 | −1 | 9 |
| TPRKLVGRA | 162 | USP35 | chr11 | 77920855 | 77920856 | C | 8 | FS | 7 | −1 | 10 |
| V | | | | | | | | | | | |
| WLPKMPPFV | 163 | R3HDM2 | chr12 | 57648749 | 57648750 | G | 13 | FS | 12 | −1 | 9 |
| SQNWGSLPL | 164 | MRI1 | chr19 | 13882967 | 13882968 | C | 6 | FS | 5 | −1 | 9 |
| GLLHAVQEK | 165 | MYO10 | chr5 | 16694605 | 16694605 | G | 8 | FS | 9 | 1 | 10 |
| L | | | | | | | | | | | |
| ATLVTPPTRY | 166 | CAD | chr2 | 27456981 | 27456981 | C | 6 | FS | 7 | 1 | 10 |
| IAFSQLIGM | 167 | TSPAN7 | chrX | 38535026 | 38535027 | C | 7 | FS | 6 | −1 | 9 |
| LSNVAPPAF | 168 | MAPRE3 | chr2 | 27248516 | 27248517 | C | 8 | FS | 7 | −1 | 9 |
| KVPFFSALK | 169 | C22orf24 | chr22 | 32334104 | 32334105 | T | 9 | FS | 8 | −1 | 9 |
| RFCPASCSGC | 170 | PLOD3 | chr7 | 100855926 | 100855927 | C | 7 | FS | 6 | −1 | 11 |
| Y | | | | | | | | | | | |
| RTHPYSPKK | 171 | USF2 | chr19 | 35761985 | 35761985 | A | 5 | inframe_ | 8 | 2 | 9 |
| | | | | | | | | ins | | | |
| VSNIAQAPLY | 172 | CLCA2 | chr1 | 86921034 | 86921036 | CT | 3 | FS | 2 | −1 | 10 |
| YVAIRPLPY | 173 | OR1K1 | chr9 | 125562787 | 125562788 | C | 10 | FS | 9 | −1 | 9 |
| MYFFWPCSL | 174 | OR52N5 | chr11 | 5799651 | 5799653 | T | 10 | FS | 8 | −1 | 9 |
| ILFFFSSK | 175 | OR7E24 | chr19 | 9361740 | 9361741 | T | 11 | FS | 10 | −1 | 8 |
| HTCKVCVSF | 176 | KLHL29 | chr2 | 23914717 | 23914720 | NA | NA | inframe_ | NA | −2 | 9 |
| | | | | | | | | del | | | |
| LAYWEKREA | 177 | ZBED6CL | chr7 | 150028142 | 150028145 | AG | 3 | inframe_ | 2 | −2 | 10 |
| W | | | | | | | | del | | | |
| HNVQGFHPY | 178 | DAZAP1 | chr19 | 1434829 | 1434830 | G | 6 | FS | 5 | −1 | 9 |
| SMAASPSPK | 179 | CACNA1G | chr17 | 48703919 | 48703920 | C | 7 | FS | 6 | −1 | 9 |
| RAFSTFPSF | 180 | EPHA10 | chr1 | 38185237 | 38185238 | C | 6 | FS | 5 | −1 | 9 |
| KSVRGLELL | 181 | ATP2B1 | chr12 | 90005129 | 90005129 | A | 5 | FS | 10 | 4 | 9 |
| QSSLSEKKF | 182 | KIF21A | chr12 | 39713783 | 39713784 | T | 9 | FS | 8 | −1 | 9 |
| RSLMSVASA | 183 | MAPK8IP1 | chr11 | 45907401 | 45907402 | G | 7 | FS | 6 | −1 | 10 |
| Y | | | | | | | | | | | |
| GTNFWGVPR | 184 | DGKD | chr2 | 234365951 | 234365952 | G | 7 | FS | 6 | −1 | 10 |
| K | | | | | | | | | | | |
| LSYNLGAGE | 185 | KCNH3 | chr12 | 49948319 | 49948320 | G | 5 | FS | 4 | −1 | 11 |
| AL | | | | | | | | | | | |
| ITSPALLL | 186 | ADAMTS17 | chr15 | 100516255 | 100516256 | C | 6 | FS | 5 | −1 | 8 |
| SSNSCASAF | 187 | ARL10 | chr5 | 175796243 | 175796243 | T | 16 | FS | 15 | 1 | 9 |
| GSYPSGSPCV | 188 | ASCL4 | chr12 | 108169096 | 108169097 | C | 5 | FS | 4 | −1 | 11 |
| W | | | | | | | | | | | |
| HSASNGTPL | 189 | ZBTB20 | chr3 | 114058002 | 114058003 | G | 7 | FS | 6 | −1 | 9 |
| KLVGRAVRR | 190 | USP35 | chr11 | 77920779 | 77920779 | C | 5 | FS | 7 | 1 | 10 |
| K | | | | | | | | | | | |
| ILKEAPRRK | 191 | ZNF541 | chr19 | 48049093 | 48049094 | C | 7 | FS | 6 | −1 | 9 |
| STSFLDTRF | 192 | TBC1D10C | chr11 | 67176564 | 67176565 | C | 7 | FS | 6 | −1 | 9 |
| KHTEKKSLSF | 193 | C4orf26 | chr4 | 76489679 | 76489680 | A | 8 | FS | 7 | −1 | 10 |
| FTHFHGEIW | 194 | CYP26A1 | chr10 | 94835039 | 94835044 | AG | 4 | FS | 3 | −4 | 9 |
| AVLGTMVMK | 195 | ADAMTSL4 | chr1 | 150530505 | 150530506 | G | 8 | FS | 7 | −1 | 9 |
| QAGTPVMMF | 196 | NEUROD4 | chr12 | 55421127 | 55421128 | C | 6 | FS | 5 | −1 | 9 |
| MTLFSLVPL | 197 | ACOX3 | chr4 | 8418187 | 8418188 | C | 4 | FS | 3 | −1 | 9 |
| SSLGTSDPRW | 198 | ACOX2 | chr3 | 58517427 | 58517428 | C | 4 | FS | 3 | −1 | 10 |
| HSLVQMEPL | 199 | ELAVL3 | chr19 | 11577604 | 11577605 | G | 9 | FS | 8 | −1 | 9 |

| | | | | | | | | | | | | | Pre- |
|---|---|---|---|---|---|---|---|---|---|---|---|---|---|
| | | | | | | | | | | | | | dicted |
| | | | Immuno- | | | | Binding | Tumor | Binding | | | Eli- | in th |
| SEQ | | | geni | | SEQ | | Affi- | Abun- | Sta- | Sample | Eli- | spot | vali- |
| ID | | | city | Wildtype | ID | HLA | nity | dance | bility | Re- | spot | reac- | dation |
| NO | HGVSc | HGVSp | Score | sequence | NO | Allele | (nM) | (TPM) | (hours) | currence | tested | tive | set |
| 24 | NM_017763.4: | NP_060233.3: | 26 | FNLQKSSLSARHPQR | 784 | HLA- | 16.7 | 79.41 | 1.66 | 8 | Tested | Yes | yes |
| | c.1976del | p.Gly659Valfs | | KRRGGPSEPTPGSRPQ | | A*02: | | | | | | | |
| | | Ter41 | | DATVHPACQ | | 01 | | | | | | | |
| 115 | NM_004947.4: | NP_004938.1: | 34851 | KGHYSLHFDAFHHPL | 877 | HLA- | 886.7 | 8.49 | 0.35 | 8 | Tested | No | yes |
| | c.5555del | p.Pro1852Glnfs | | GDTPPALPARTLRKSP | | A*03: | | | | | | | |
| | | Ter45 | | LHPIPASPT | | 01 | | | | | | | |
| 116 | NM_ | NP_001036783.1: | 14559 | PLGQSHLAHHSMAPY | 878 | HLA- | 5 | 9.23 | 7.17 | 7 | Tested | No | No |
| | 001043318.1: | p.Asn314Thrfs | | PFPPNPDMNPELRKA | | A*02: | | | | | | | |
| | c.939del | Ter4 | | LLQDSAPQPA | | 01 | | | | | | | |
| 44 | NM_ | NP_001263181.1: | 178 | ATYVTFSPNGTELLV | 804 | HLA- | 4.4 | 10.01 | 4.97 | 6 | Tested | Yes | No |
| | 001276252.1: | p.Glu290Asnfs | | NMGGEQVYLFDLTY | | A*02: | | | | | | | |
| | c.868del | Ter8 | | KQRPYTFLLPR | | 01 | | | | | | | |
| 117 | NM_001204.6: | NP_001195.2: | 3845 | KNISSEHSMSSTPLTIG | 879 | HLA- | 14.5 | 6.59 | 6.63 | 6 | Tested | No | No |
| | c.1748del | p.Asn583Thrfs | | EKNRNSINYERQQAQ | | B*07: | | | | | | | |
| | | Ter44 | | ARIPSPET | | 02 | | | | | | | |
| 118 | NM_015241.2: | NP_056056.2: | 7352 | EPNASVVPPPLPATW | 880 | HLA- | 22.8 | 28.06 | 0.28 | 6 | Tested | No | No |
| | c.4495del | p.Arg1499Glyfs | | MRPPREPAQPPREEV | | C*16: | | | | | | | |
| | | Ter106 | | RKSFVESVEE | | 01 | | | | | | | |
| 119 | NM_ | NP_001275879.1: | 24474 | SYAPAEIFLPKGRSNS | 881 | HLA- | 372.5 | 4.09 | 0.16 | 6 | Tested | No | No |
| | 001288950.1: | p.Arg657Glyfs | | KKKRQKKQNTSCSK | | C*16: | | | | | | | |
| | c.1969del | Ter33 | | NRGRTTAHTK | | 01 | | | | | | | |
| 120 | NM_031433.3: | NP_113621.1: | 37818 | SSSGAFSLLGRFCGAE | 882 | HLA- | 7.2 | 1.00E−04 | 0.84 | 6 | Tested | No | No |
| | c.1150del | p.His384Thrfs | | PPPHLVSSHHELAVLF | | B*15: | | | | | | | |
| | | Ter94 | | RTDHGISS | | 17 | | | | | | | |
| 121 | NM_ | NP_001005500.1: | 48030 | TIMNRRLCCILVALS | 883 | HLA- | 32.3 | 1.86 | 0.95 | 6 | Tested | No | No |
| | 001005500.1: | p.Phe153Leufs | | WMGGFIHSIIQVALIV | | A*30: | | | | | | | |
| | c.455dup | Ter21 | | RLPFCGPNE | | 02 | | | | | | | |
| 27 | NM_005650.2: | NP_005641.1: | 42 | NFSVRCPKHKPPLPCP | 787 | HLA- | 4.8 | 39.27 | 0.98 | 5 | Tested | Yes | No |
| | c.5826del | p.Leu1943Cysfs | | LPPLQNKTAKGSLSTE | | B*07: | | | | | | | |
| | | Ter118 | | QSERG | | 02 | | | | | | | |
| 28 | NM_ | NP_001139746.1: | 53 | ALFGLDRQTLWCKPC | 788 | HLA- | 5.1 | 44.54 | 0.35 | 5 | Tested | No | No |
| | 001146274.1: | p.Lys468Serfs | | RRKKKCVRYIQGEGS | | C*03: | | | | | | | |
| | c.1403del | Ter23 | | CLSPPSSDGS | | 03 | | | | | | | |
| 122 | NM_ | NP_001020018.1: | 1605 | HDFILEDAASPKCIMK | 884 | HLA- | 44.8 | 7.63 | 0.73 | 5 | Tested | No | No |
| | 001024847.2: | p.Lys153Serfs | | EKKKPGETFFMCSCS | | B*15: | | | | | | | |
| | c.458del | Ter35 | | SDECNDNII | | 03 | | | | | | | |
| 123 | NM_ | NP_001005271.2: | 2604 | PVAVPAPQQADGNPD | 885 | HLA- | 110.3 | 98.14 | 0.17 | 5 | Tested | No | No |
| | 001005271.2: | p.Arg599Valfs | | VPPPRPLQGRSEREFF | | C*05: | | | | | | | |
| | c.1795del | Ter16 | | VKWVGLSYW | | 01 | | | | | | | |
| 124 | NM_006015.4: | NP_006006.3: | 5556 | LGRVQEFDSGLLHWR | 886 | HLA- | 18.2 | 67.42 | 0.69 | 5 | Tested | No | No |
| | c.5548del | p.Asp1850Thrfs | | IGGGDTTEHIQTHFES | | B*15: | | | | | | | |
| | | Ter33 | | KTELLPSRP | | 17 | | | | | | | |
| 125 | NM_0151552: | NP_055970.1: | 5649 | GGNESQPDSQEDPRE | 887 | HLA- | 29 | 20.21 | 3.21 | 5 | Tested | No | No |
| | c.487del | p.Thr163Hisfs | | VLKKTLEFCLSRENL | | A*30: | | | | | | | |
| | | Ter47 | | ASDMYLISQM | | 02 | | | | | | | |
| 126 | NM_002356.5: | NP_002347.5: | 6932 | PKAEDGATPSPSNETP | 888 | HLA- | 9.4 | 2.09 | 0.54 | 5 | Tested | No | yes |
| | c.464del | p.Lys155Argfs | | KKKKKRFSFKKSFKL | | B*15: | | | | | | | |
| | | Ter12 | | SGFSFKKNK | | 17 | | | | | | | |
| 127 | NM_ | NP_ | 10038 | EIGQHPSLEDMQEVV | 889 | HLA- | 370.4 | 10.38 | 0.35 | 5 | Tested | No | No |
| | 001278579.1: | 001265508.1: | | VHKKKRPVLRDYWQ | | B*15: | | | | | | | |
| | c.1310del | p.Lys437Argfs | | KHAGMAMLCET | | 17 | | | | | | | |
| | | Ter5 | | | | | | | | | | | |
| 128 | NM_ | NP_ | 37508 | ATIMNQRLCCILVALS | 890 | HLA- | 14.2 | 1.00E−04 | 0.8 | 5 | No | N/A | No |
| | 001004719.2: | 001004719.2: | | WRGGFIHSIIQVALIV | | B*15: | | | | | | | |
| | c.455del | p.Gly152Alafs | | RLPFCGPN | | 17 | | | | | | | |
| | | Ter23 | | | | | | | | | | | |
| 129 | NM_020782.1: | NP_065833.1: | 43379 | HIRKQQMVSVEETIYI | 891 | HLA- | 17.2 | 1.00E−04 | 0.77 | 5 | No | N/A | No |
| | c.1194del | p.Cys399Valfs | | VGGCLHELGPNRRSS | | B*15: | | | | | | | |
| | | Ter44 | | QSEDMLTVQ | | 17 | | | | | | | |
| 130 | NM_178537.4: | NP_848632.2: | 43649 | SDFDRVGGMNTEEFR | 892 | HLA- | 65.2 | 1.00E−04 | 0.22 | 5 | No | N/A | No |
| | c.2972del | p.Gly991Valfs | | DQWGGEDWELLDRV | | B*15: | | | | | | | |
| | | Ter71 | | LQAGLEVERLR | | 17 | | | | | | | |
| 37 | NM_015902 | NP_056986.2: | 119 | MSYAANLKNVMNM | 797 | HLA- | 6.4 | 61.73 | 10.18 | 4 | Tested | Yes | No |
| | c.6360del | p.Glu2121Lysfs | | QNRQKKEGEEQPVLP | | A*03: | | | | | | | |
| | | Ter28 | | EETESSKPGPSA | | 01 | | | | | | | |
| 58 | NM_001283.3: | NP_001274.1: | 360 | IIFNFEKAYFILDEFL | 818 | HLA- | 21.6 | 36.11 | 0.41 | 4 | Tested | No | No |
| | c.364del | p.Asp122Metfs | | MGGDVQDTSKKSVLK | | B*15: | | | | | | | |
| | | Ter11 | | AIEQADLLQ | | 17 | | | | | | | |
| 131 | NM_020799.3: | NP_065850.1: | 1679 | TGIFRLTNAGMLEVS | 893 | HLA- | 51.9 | 12.7 | 3.17 | 4 | Tested | No | No |
| | c.1214del | p.Lys405Argfs | | ACKKKGFHPHTKEPR | | B*27: | | | | | | | |
| | | Ter21 | | LFSICKHVLV | | 05 | | | | | | | |
| 132 | NM_ | NP_ | 1814 | VPRVEGVFIFLIEDSG | 894 | HLA- | 6.8 | 10.67 | 0.61 | 4 | Tested | No | No |
| | 001136112.1: | 001129584.1: | | KKKRRKNFEAMFKGI | | B*15: | | | | | | | |
| | c.67de1 | p.Arg23Glyfs | | LqSGLDNFV | | 03 | | | | | | | |
| | | Ter21 | | | | | | | | | | | |
| 133 | NM_012231.4: | NP_036363.2: | 2434 | NKHAAFSCPKKPLSP | 895 | HLA- | 57.6 | 41.67 | 0.22 | 4 | Tested | No | No |
| | c.4467del | p.Val1490Phefs | | PKKKVSHSSKKGGHS | | C*05: | | | | | | | |
| | | Ter74 | | SPASSDKNSN | | 01 | | | | | | | |
| 134 | NM_014246.1: | NP_055061.1: | 3456 | GENARLHYRLVDTAS | 896 | HLA- | 9.6 | 8.83 | 3.8 | 4 | Tested | No | No |
| | c.1841del | p.Gly614Alafs | | TFLGGGSAGPKNPAP | | B*07: | | | | | | | |
| | | Ter54 | | TPDFPFQIHN | | 02 | | | | | | | |
| 5 | NM_002356.5: | NP_002347.5: | 5157 | PKAEDGATPSPSNETP | 888 | HLA- | 51.5 | 6 | 5.59 | 4 | Tested | No | No |
| | c.463_464del | p.Lys155Glufs | | KKKKKRFSFKKSFKL | | A*02: | | | | | | | |
| | | Ter28 | | SGFSFKKNK | | 01 | | | | | | | |
| 135 | NM_ | NP_ | 6489 | ESKFKSRASNAQAKP | 897 | HLA- | 136.9 | 10.58 | 0.17 | 4 | Tested | No | No |
| | 001278458.1: | 001265387.1: | | SSFFLQMQKRVSGHY | | C*06: | | | | | | | |
| | c.2921del | p.Leu974Cysfs | | VTSAAAKSVH | | 02 | | | | | | | |
| | | Ter12 | | | | | | | | | | | |
| 136 | NM_ | NP_ | 8277 | SSQLLTPAERPGGLD | 898 | HLA- | 15.2 | 4.54 | 1.19 | 4 | Tested | Yes | yes |
| | 001184772.2: | 001171701.1: | | DRSPPGSSETVELVRY | | B*15: | | | | | | | |
| | c.5264del | p.Pro1755Glnfs | | EPDLLRLLG | | 17 | | | | | | | |
| | | Ter20 | | | | | | | | | | | |
| 137 | NM_ | NP_ | 10241 | NCHISLTPNGDMPGS | 899 | HLA- | 3.5 | 57.53 | 14.24 | 4 | Tested | No | No |
| | 001321148.1: | 001308077.1: | | EIPPSSPSHAGSLHDD | | B*07: | | | | | | | |
| | c.2712de | p.Ser905Profs | | LNQVSRDDA | | 02 | | | | | | | |
| | | Ter13 | | | | | | | | | | | |
| 138 | NM_005116.5: | NP_005107.4: | 15302 | ESIGDYYACARLSCA | 900 | HLA- | 44.4 | 3.56 | 5.9 | 4 | Tested | Yes | No |
| | c.1233del | p.Ile412Serfs | | PPPPIHAINRGIFVEGL | | A*02: | | | | | | | |
| | | Ter4 | | SCVLDGIF | | 01 | | | | | | | |
| 139 | NM_133448.2: | NP_597705.2: | 29938 | DLGLCVAELELLSSW | 901 | HLA- | 25.6 | 1.00E−04 | 0.57 | 4 | Tested | No | No |
| | c.618del | p.Thr207Argfs | | FSPPTVVAGRRKSVD | | B*15: | | | | | | | |
| | | Ter75 | | QPEGTPVELY | | 17 | | | | | | | |
| 140 | NM_0808662: | NP_543142.2: | 32386 | TLEILKSTMKKELEA | 902 | HLA- | 2.7 | 1.00E−04 | 0.79 | 4 | Tested | No | yes |
| | c.1005del | p.Lys335Asnfs | | AQKKKPSLCEMLHM | | B*15: | | | | | | | |
| | | Ter67 | | PNICKRISLLS | | 17 | | | | | | | |
| 141 | NM_ | NP_ | 36344 | PCFYPDEDDFYFGGP | 903 | HLA- | 11.6 | 1.00E−04 | 0.34 | 4 | Tested | No | No |
| | 001293228.1: | 001280157.1: | | DSTPPGEDIWKKFELL | | B*15: | | | | | | | |
| | c.134del | p.Pro45Argfs | | PTPPLSPSR | | 17 | | | | | | | |
| | | Ter86 | | | | | | | | | | | |
| 142 | NM_ | NP_ | 37186 | TGALLLQGFIQDRAG | 904 | HLA- | 4.6 | 1.00E−04 | 1.86 | 4 | No | N/A | No |
| | 001291428.1: | 001278357.1: | | RMGGEAPELALDPVP | | B*15: | | | | | | | |
| | c.121del | p.Glu41Argfs | | QDASTKKLSE | | 17 | | | | | | | |
| | | Ter19 | | | | | | | | | | | |
| 143 | NM_007118.2: | NP_009049.2: | 40065 | STSRSRPSRIPQPVRH | 905 | HLA- | 4.5 | 1.00E−04 | 0.84 | 4 | No | N/A | No |
| | c.7050del | p.Val2351Cysfs | | HPPVLVSSAASSQAE | | B*15: | | | | | | | |
| | | Ter62 | | ADKMSGTST | | 17 | | | | | | | |
| 144 | NM_006617.1: | NP_006608.1: | 40265 | NQEFLQARTPTLASTP | 906 | HLA- | 8.4 | 1.00E−04 | 1.84 | 4 | No | N/A | No |
| | c.1176del | p.Thr393Hisfs | | IPPTPQAPSPAVDAEIR | | B*15: | | | | | | | |
| | | Ter9 | | AQDAPLS | | 17 | | | | | | | |
| 145 | NM_005357.2: | NP_005348.2: | 42706 | GAGPSGETGAAGVD | 907 | HLA- | 11.6 | 1.00E−04 | 0.73 | 4 | No | N/A | No |
| | c.3222del | p.Arg1075Aspfs | | GGCGGRH | | B*15: | | | | | | | |
| | | Ter101 | | | | 17 | | | | | | | |
| 146 | NM_0165294: | NP_057613.4: | 42835 | GQEQTFGILNVLEFSS | 908 | HLA- | 266.5 | 1.00E−04 | 0.29 | 4 | No | N/A | No |
| | c.1719_ | p.Arg575Lysfs | | DRKRMSVIVRTPSGR | | A*03: | | | | | | | |
| | 1723dup | Ter6 | | LRLYCKGAD | | 26 | | | | | | | |
| 147 | NM_025004.2: | NP_079280.2: | 43220 | ETMKQARHRLASFKT | 909 | HLA- | 66.4 | 1.00E−04 | 0.42 | 4 | No | N/A | No |
| | c.581del | p.Lys194Argfs | | VIKKKGSVFPDDGRK | | B*15: | | | | | | | |
| | | Ter29 | | SFLTREEVLS | | 17 | | | | | | | |
| 148 | NM_004938.3: | NP_004929.2: | 47201 | DFFRAQTLKETSLTNT | 910 | HLA- | 344.3 | 1.00E−04 | 0.16 | 4 | No | N/A | No |
| | c.3822dup | p.Tyr1275Valfs | | MGGYKESFSSIMCFG | | A*03: | | | | | | | |
| | | Ter64 | | CHDVYSQAS | | 26 | | | | | | | |
| 149 | NM_032936.3: | NP_116325.1: | 52134 | GRCKSGFDPRHGSHN | 911 | HLA- | 199.5 | 1.22 | 0.42 | 4 | No | N/A | No |
| | c.231del | p.Ala78Profs | | IKKKAWYLIAMLLKL | | A*30: | | | | | | | |
| | | Ter11 | | AFCLALCAKL | | 02 | | | | | | | |
| 20 | NM_ | NP_ | 8 | GNKCTMCKEKLERE | 779 | HLA- | 12.2 | 461.65 | 1.06 | 3 | Tested | Yes | yes |
| | 001127698.1: | 001121170.1: | | AAEKKKKEDEDRSNT | | B*15: | | | | | | | |
| | c.2468del | p.Lys823Argfs | | GERSNTGERSN | | 17 | | | | | | | |
| | | Ter119 | | | | | | | | | | | |
| 29 | NM_ | NP_ | 62 | DQLQQAVQSQGFINY | 789 | HLA- | 14.9 | 24.08 | 1.66 | 3 | Tested | Yes | yes |
| | 001318120.1: | 001305049.1: | | CQKKIDASQTEFEKN | | A*03: | | | | | | | |
| | c.1384del | p.Ile462Leufs | | VWSFLKVNFE | | 01 | | | | | | | |
| | | Ter16 | | | | | | | | | | | |
| 38 | NM_ | NP_001243000.2: | 143 | HKDAWRQPEDTWAA | 798 | HLA- | 26.5 | 105.69 | 0.35 | 3 | Tested | No | No |
| | 001256071.2: | p.Phe727del | | LEGLSFSPFREQMLDT | | B*15: | | | | | | | |
| | c.2180_ | | | SSLLQFMREK | | 17 | | | | | | | |
| | 2182del | | | | | | | | | | | | |
| 57 | NM_ | NP_001230368.1: | 345 | SFGSPTGNQMSSDIDE | 817 | HLA- | 19.8 | 25.32 | 2.51 | 3 | Tested | No | No |
| | 001243439.1: | p.Asn303Thrfs | | YKKNIHGNALRTSGS | | B*15: | | | | | | | |
| | c.908del | Ter63 | | SSSDVTKAS | | 17 | | | | | | | |
| 62 | NM_ | NP_ | 391 | CSIERADNDKEYLVL | 822 | HLA- | 17.1 | 66.83 | 0.62 | 3 | Tested | Yes | No |
| | 001304717.2: | 001291646.2: | | TLTKNDLDKANKDK | | B*15: | | | | | | | |
| | c.1487del | p.Asn496Metfs | | ANRYFSPNFKV | | 17 | | | | | | | |
| | | Ter21 | | | | | | | | | | | |
| 95 | NM_ | NP_001164638.1: | 935 | DGTFSVTSAYSSAPD | 856 | HLA- | 76 | 58.89 | 2.29 | 3 | Tested | No | yes |
| | 001171167.1: | p.Pro889Leufs | | GSPPPAPLPASEMTM | | B*07: | | | | | | | |
| | c.2666del | Ter9 | | EDMAPGQLSS | | 02 | | | | | | | |
| 97 | NM_0221672: | NP_071450.2: | 969 | VNQEVLEILDFHLYG | 859 | HLA- | 7.6 | 10.17 | 19.8 | 3 | Tested | No | yes |
| | c.1584del | p.Gly529Alafs | | SYPPGTPALKAYWEN | | B*07: | | | | | | | |
| | | Ter78 | | TYDAADGPSG | | 02 | | | | | | | |
| 106 | NM_001305.3: | NP_001296.1: | 1072 | GASLYVGWAASGLL | 868 | HLA- | 20.5 | 19.67 | 0.41 | 3 | Tested | No | No |
| | c.537del | p.Leu180Cysfs | | LLGGGLLCCNCPPRT | | B*15: | | | | | | | |
| | | Ter115 | | DKPYSAKYSAA | | 17 | | | | | | | |
| 150 | NM_ | NP_001243727.1: | 4368 | DGLRSRVKYGVKTTP | 912 | HLA- | 11.2 | 4.6 | 3.86 | 3 | Tested | No | No |
| | 001256798.1: | p.Tyr377Thrfs | | ESPPYSSGSYDSIKTE | | A*02: | | | | | | | |
| | c.1128del | Ter20 | | VSGCPEDLT | | 01 | | | | | | | |
| 151 | NM_030919.2: | NP_112181.2: | 4370 | SIRTTDFHNPGYPKYL | 913 | HLA- | 141.4 | 20.3 | 1.32 | 3 | Tested | Yes | No |
| | c.1633del | p.His545Thrfs | | GTPHLELYLSDSLRNL | | A*02: | | | | | | | |
| | | Ter6 | | NKERQFHF | | 01 | | | | | | | |
| 152 | NM_ | NP_001284576.1: | 4500 | REPAGLSLVLKKIPIPE | 914 | HLA- | 30.4 | 42.13 | 1.14 | 3 | Tested | No | No |
| | 001297647.1: | p.Pro291Hisfs | | TPPQTPPQVLDSPHQR | | B*27: | | | | | | | |
| | c.872del | Ter74 | | SPSLSLA | | 05 | | | | | | | |
| 153 | NM_001852.3: | NP_001843.1: | 4763 | PQGLPGVKGDKGSPG | 915 | HLA- | 8 | 52.16 | 0.64 | 3 | Tested | No | yes |
| | c.1312del | p.Arg438Alafs | | KTGPRGKVGDPGVA | | B*15: | | | | | | | |
| | | Ter93 | | GLPGEKGEKGE | | 17 | | | | | | | |
| 154 | NM_015477.2: | NP_056292.1: | 5021 | TAPSLQNNQPVEFNH | 916 | HLA- | 10.9 | 9.22 | 2.09 | 3 | Tested | No | No |
| | c.931del | p.Val311Leufs | | AINYVNKIKNRFQGQ | | A*31: | | | | | | | |
| | | Ter43 | | PDIYKAFLEI | | 01 | | | | | | | |
| 155 | NM_002631.3: | NP_002622.2: | 5554 | AVSTGVQAGIPMPCF | 917 | HLA- | 241.4 | 88.16 | 0.5 | 3 | Tested | No | No |
| | c.1282_ | p.Ser428Leufs | | TTALSFYDGYRHEML | | B*51: | | | | | | | |
| | 1283del | Ter3 | | PASLIQAQRD | | 01 | | | | | | | |
| 156 | NM_016441.2: | NP_057525.1: | 5940 | CTHCYCLQGQTLCST | 918 | HLA- | 6 | 20.02 | 1.2 | 3 | Tested | Yes | No |
| | c.2567del | p.Pro856Leufs | | VSCPPLPCVEPINVEG | | B*15: | | | | | | | |
| | | Ter67 | | SCCPMCPEM | | 17 | | | | | | | |
| 157 | NM_ | NP_ | 6022 | GTMRATGDFVTVKD | 919 | HLA- | 59.4 | 12.85 | 3.25 | 3 | Tested | Yes | No |
| | 001323890.1: | 001310819.1: | | GEIFFLGRKDSQIKRH | | A*02: | | | | | | | |
| | c.1319del | p.Leu440Trpfs | | GKRLNIELVQ | | 01 | | | | | | | |
| | | Ter43 | | | | | | | | | | | |
| 158 | NM_ | NP_001308758.1: | 6671 | LSLEINRKLQAVLEDT | 920 | HLA- | 347.5 | 32.63 | 1.35 | 3 | Tested | Yes | No |
| | 001321829.1: | p.Asn867Ilefs | | LLKNITLKENLQTLGT | | A*02: | | | | | | | |
| | c.2600del | Ter4 | | EIERLIKH | | 01 | | | | | | | |
| 159 | NM_ | NP_ | 7375 | PMAFSPQRDRFQAEG | 921 | HLA- | 4.1 | 5.64 | 14.19 | 3 | Tested | No | No |
| | 001134419.1: | 001127891.1: | | SLKKNEQNFKLAGVK | | A*02: | | | | | | | |
| | c.92de1 | p.Asn31Thrfs | | KDIEKLYEAV | | 01 | | | | | | | |
| | | Ter51 | | | | | | | | | | | |
| 160 | NM_005523.5: | NP_005514.1: | 7459 | LTDRQVKIWFQNRR | 922 | HLA- | 60.7 | 11.57 | 0.68 | 3 | Tested | Yes | No |
| | c.895del | p.Ile299Leufs | | MKEKKINRDRLQYYS | | A*30: | | | | | | | |
| | | Ter30 | | ANPLL | | 02 | | | | | | | |
| 161 | NM_006887.4: | NP_008818.3: | 7672 | GSAAAGGPTSYGTLK | 923 | HLA- | 408.7 | 118.56 | 1.56 | 3 | Tested | No | No |
| | c.320del | p.Gly107Alafs | | EPSGGGGTALLNKEN | | A*02: | | | | | | | |
| | | Ter80 | | KFRDRSFSEN | | 01 | | | | | | | |
| 162 | NM_020798.2: | NP_065849.1: | 9425 | GRVGPRRQRKHCITE | 924 | HLA- | 14.7 | 4.34 | 4.09 | 3 | Tested | No | No |
| | c.1962del | p.Thr655Profs | | DTPPTSLYIEGLDSKE | | B*07: | | | | | | | |
| | | Ter23 | | AGGQSSQEE | | 02 | | | | | | | |
| 163 | NM_001330 | NP_00131705 | 10387 | GAKIQWLKDAQGLP | 925 | HLA- | 13.3 | 37.29 | 1.71 | 3 | Tested | Yes | No |
| | 121.1:c.283 | 0.1:p.Asp947 | | GGGGGDNSGTAENG | | A*02: | | | | | | | |
| | 9del | ThrfsTer41 | | RHSDLAALYTIV | | 01 | | | | | | | |
| 164 | NM_ | NP_001026897.1: | 13130 | VRIAAPGIGVWNPAF | 926 | HLA- | 58.3 | 4.31 | 4.76 | 3 | Tested | No | No |
| | 001031727.2: | p.His330Thrfs | | DVTPHDLITGGIITEL | | B*15: | | | | | | | |
| | c.988del | Ter24 | | GVFAPEELR | | 01 | | | | | | | |
| 165 | NM_012334.2: | NP_036466.2: | 18568 | FRSKQEALKQGWLH | 927 | HLA- | 232 | 37.55 | 2.5 | 3 | Tested | No | No |
| | c.3674dup | p.Ser1226Leufs | | KKGGGSSTLSRRNWK | | A*02: | | | | | | | |
| | | Ter25 | | KRWFVLRQSKL | | 01 | | | | | | | |
| 166 | NM_004341.4: | NP_004332.2: | 21546 | EHVENAGVHSGDATL | 928 | HLA- | 164.4 | 1.00E−04 | 0.91 | 3 | Tested | No | No |
| | c.3512dup | p.Gln1172Thrfs | | VTPPQDITAKTLERIK | | A*30: | | | | | | | |
| | | Ter37 | | AIVHAVGQE | | 02 | | | | | | | |
| 167 | NM_004615.3: | NP_004606.2: | 23216 | QNYTNWSTSPYFLEH | 929 | HLA- | 19.1 | 27.73 | 0.24 | 3 | Tested | No | No |
| | c.516del | p.Ser173Alafs | | GIPPSCCMNETDCNP | | C*03: | | | | | | | |
| | | Ter4 | | QDLHNLTVAA | | 04 | | | | | | | |
| 168 | NM_012326.2: | NP_036458.2: | 23926 | PTGPKNMQTSGRLSN | 930 | HLA- | 6.7 | 1.00E−04 | 0.81 | 3 | Tested | Yes | No |
| | c.543del | p.Cys182Alafs | | VAPPCILRKNPPSARN | | B*15: | | | | | | | |
| | | Ter31 | | GGHETDAQI | | 17 | | | | | | | |
| 169 | NM_ | NP_ | 26241 | DFLSVKWEAAMMNG | 931 | HLA- | 25.6 | 1.00E−04 | 1.31 | 3 | Tested | No | yes |
| | 001302819.1: | 001289748.1: | | KVPFFFSSESLGYFAT | | A*03: | | | | | | | |
| | c.149del | p.Phe50Serfs | | GRPADNVMTT | | 26 | | | | | | | |
| | | Ter6 | | | | | | | | | | | |
| 170 | NM_001084.4: | NP_001075.1: | 27472 | GCGFCNQDRRTLPGG | 932 | HLA- | 186.1 | 40.29 | 3.03 | 3 | Tested | No | No |
| | c.889del | p.Arg297Glyfs | | QPPPRVFLAVFVEQPT | | A*30: | | | | | | | |
| | | Ter61 | | PFLPRFLQR | | 02 | | | | | | | |
| 171 | NM_003367.3: | NP_003358.1: | 28137 | VLQTGTORTIAPRTHP | 933 | HLA- | 26.2 | 1.00E−04 | 11.31 | 3 | Tested | No | No |
| | c.671_673dup | p.Lys224dup | | YSPKIDGTRTPRDERR | | A*03: | | | | | | | |
| | | | | RAQHNEVE | | 26 | | | | | | | |
| 172 | NM_006536.5: | NP_006527.1: | 29458 | AMDRNSLQSAVSNIA | 934 | HLA- | 14.5 | 1.00E−04 | 1.78 | 3 | Tested | No | No |
| | c.2658_ | p.Phe887Tyrfs | | QAPLFIPPNSDPVPAR | | B*15: | | | | | | | |
| | 2659del | Ter6 | | DYLILKGVL | | 17 | | | | | | | |
| 173 | NM_080859.1: | NP_543135.1: | 30248 | DSCLLAAMAYDCYV | 935 | HLA- | 36.3 | 1.00E−04 | 1.06 | 3 | Tested | No | No |
| | c.391del | p.Leu131Serfs | | AIRHPLPYATRMSRA | | B*15: | | | | | | | |
| | | Ter56 | | MCAALVGMAWL | | 17 | | | | | | | |
| 174 | NM_ | NP_ | 32050 | GLVYLIYYEESLHHP | 936 | HLA- | 69.8 | 1.00E−04 | 0.16 | 3 | Tested | No | No |
| | 001001922.2: | 001001922.2: | | MYFFFGHALSLIDLLT | | C*07: | | | | | | | |
| | c.212213del | p.Phe71Trpfs | | CTTTLPNAL | | 01 | | | | | | | |
| | | Ter8 | | | | | | | | | | | |
| 175 | NM_ | NP_001073404.1: | 32261 | MSYFPILFFFFLKRCP | 937 | HLA- | 90.9 | 1.00E−04 | 2.32 | 3 | Tested | No | No |
| | 001079935.1: | p.Phe11Serfs | | SYTEPQNLTGVSEFL | | A*03: | | | | | | | |
| | c.32de1 | Ter89 | | | | 26 | | | | | | | |
| 176 | NM_052920.1: | NP_443152.1: | 34025 | DSANAKTLLEAASKF | 938 | HLA- | 51.8 | 1.00E−04 | 1.13 | 3 | Tested | No | No |
| | c.1256_1 | p.Phe419del | | QFHTFCKVCVSFLEK | | B*15: | | | | | | | |
| | 258del | | | QLTASNCLGV | | 17 | | | | | | | |
| 177 | NM_138434.2: | NP_612443.1: | 34279 | LWENETVGAQDDPL | 939 | HLA- | 80.4 | 1.00E−04 | 0.39 | 3 | Tested | No | No |
| | c.655_657del | p.Lys219del | | AYWEKKREAWPPSIC | | B*15: | | | | | | | |
| | | | | LTPHRSLL | | 17 | | | | | | | |
| 178 | NM_018959.2: | NP_061832.2: | 34622 | SDPSQQPPSYGGPSVP | 940 | HLA- | 204.4 | 35.22 | 0.32 | 3 | Tested | No | No |
| | c.1148del | p.Gly383Alafs | | GSGGPPAGGSGFGRG | | A*30: | | | | | | | |
| | | Ter46 | | QNHNVQGFH | | 02 | | | | | | | |
| 179 | NM_018896.4: | NP_061496.2: | 34903 | PGSRPKKKLSPPSITID | 941 | HLA- | 16.4 | 1.00E−04 | 3.28 | 3 | Tested | No | No |
| | c.6948del | p.Glu2317Argfs | | PPESQGPRTPPSPGICL | | A*03: | | | | | | | |
| | | Ter47 | | RRRAPS | | 26 | | | | | | | |
| 180 | NM_ | NP_001092909.1: | 35607 | MSGQDVIKAVEDGFR | 942 | HLA- | 2.8 | 1.00E−04 | 1.29 | 3 | No | N/A | No |
| | 001099439.1: | p.Arg869Glyfs | | LPPPRNCPNLLHRLM | | B*15: | | | | | | | |
| | c.2604del | Ter10 | | LDCWQKDPGE | | 17 | | | | | | | |
| 181 | NM_001682.2: | NP_001673.2: | 36934 | AVVGIEDPVRPEVPD | 943 | HLA- | 23.8 | 1.00E−04 | 0.73 | 3 | No | N/A | No |
| | c.2083_ | p.Cys697Lysfs | | AIKKCQRAGITVRMV | | B*15: | | | | | | | |
| | 2087dup | Ter40 | | TGDNINTARA | | 17 | | | | | | | |
| 182 | NM_ | NP_001166935.1: | 37118 | GLPSKIGSISRQSSLSE | 944 | HLA- | 150.5 | 1.00E−04 | 0.23 | 3 | No | N/A | No |
| | 001173464.1: | p.Ile1235Phefs | | KKIPEPSPVTRRKAYE | | B*15: | | | | | | | |
| | c.3703del | Ter7 | | KAEKSKA | | 17 | | | | | | | |
| 183 | NM_005456.3: | NP_005447.1: | 37475 | MAERESGGLGGGAA | 945 | HLA- | 5.1 | 1.00E−04 | 2.26 | 3 | No | N/A | No |
| | c.37del | p.Ala13Profs | | SPPAASPFLGLHIASPP | | B*15: | | | | | | | |
| | | Ter84 | | NF | | 17 | | | | | | | |
| 184 | NM_152879.2: | NP_690618.2: | 37681 | QGIAVLNIPSYAGGTN | 946 | HLA- | 32.5 | 1.00E−04 | 2.28 | 3 | No | N/A | yes |
| | c.2564del | p.Gly855Valfs | | FWGGTKEDDTFAAPS | | A*03: | | | | | | | |
| | | Ter48 | | FDDKILEVV | | 26 | | | | | | | |
| 185 | NM_012284.1: | NP_036416.1: | 38064 | APRFSRGLRGELSYN | 947 | HLA- | 128.8 | 1.00E−04 | 0.74 | 3 | No | N/A | No |
| | c.2123del | p.Gly708Glufs | | LGAGGGSAEVDTSSL | | B*15: | | | | | | | |
| | | Ter11 | | SGDNTLMSTL | | 17 | | | | | | | |
| 186 | NM_139057.3: | NP_620688.2: | 38186 | QCYQEVCNDRINANT | 948 | HLA- | 134.2 | 1.00E−04 | 1.27 | 3 | No | N/A | No |
| | c.3121del | p.Arg1041Alafs | | ITSPRLAALTYKCTRD | | B*15: | | | | | | | |
| | | Ter5 | | QWTVYCRVI | | 17 | | | | | | | |
| 187 | NM_ | NP_001304877.1: | 38429 | IIIKCLLYARHGVLFLF | 949 | HLA- | 24.6 | 1.00E−04 | 0.46 | 3 | No | N/A | No |
| | 001317948.1: | p.Ter277Leufs | | FF | | B*15: | | | | | | | |
| | c.829dup | Ter129 | | | | 17 | | | | | | | |
| 188 | NM_2034362: | NP_982260.2: | 39051 | RTAPLGVPGTLPGLPR | 950 | HLA- | 18.3 | 1.00E−04 | 1.15 | 3 | No | N/A | No |
| | c.109del | p.Leu37Serfs | | RDPLRVALRLDAAC | | B*15: | | | | | | | |
| | | Ter53 | | WEWARSGCAR | | 17 | | | | | | | |
| 189 | NM_ | NP_001157814.1: | 39640 | HKTLLERHVALHSAS | 951 | HLA- | 100.1 | 1.00E−04 | 0.47 | 3 | No | N/A | yes |
| | 001164342.2: | p.Pro692Leufs | | NGTPPAGTPPGARAG | | B*15: | | | | | | | |
| | c.2075del | Ter43 | | PPGVVACTEG | | 17 | | | | | | | |
| 190 | NM_020798.2: | NP_065849.1: | 39946 | RLGSVMRPTEDITAR | 952 | HLA- | 45.7 | 1.00E−04 | 5.77 | 3 | No | N/A | No |
| | c.1882_ | p.Pro629Hisfs | | ELPPPTSAQGPGRVGP | | A*03: | | | | | | | |
| | 1883dup | Ter50 | | RRQRKHCIT | | 26 | | | | | | | |
| 191 | NM_ | NP_001264004.1: | 40014 | SLRRHYEVHHGLCIL | 953 | HLA- | 204.2 | 1.00E−04 | 1.11 | 3 | No | N/A | No |
| | 001277075.1: | p.Pro231Argfs | | KEAPPEEEACGDSPH | | A*03: | | | | | | | |
| | c.692del | Ter49 | | AHESAGQPPP | | 26 | | | | | | | |
| 192 | NM_198517.3: | NP_940919.1: | 40080 | RGACPGLLETLGALR | 954 | HLA- | 9.4 | 1.00E−04 | 0.36 | 3 | No | N/A | No |
| | c.960del | p.Ala321Argfs | | AIPPAQLQEEAFMSQ | | B*15: | | | | | | | |
| | | Ter100 | | VHSVVLSERD | | 17 | | | | | | | |
| 193 | NM_ | NP_001193910.1: | 41224 | AHLRKAEREEKPKHT | 955 | HLA- | 180.7 | 1.00E−04 | 0.33 | 3 | No | N/A | No |
| | 001206981.1: | p.Ser159Alafs | | EAKKSLSFRKKQQKD | | B*15: | | | | | | | |
| | c.475del | Ter26 | | FCFIFRN | | 17 | | | | | | | |
| 194 | NM_000783.3: | NP_000774.2: | 41961 | AGQGCKDALQLLIEH | 956 | HLA- | 6.2 | 1.00E−04 | 0.93 | 3 | No | N/A | No |
| | c.843_847del | p.Gly282Alafs | | SWERGERLDMQALK | | B*15 | | | | | | | |
| | | Ter50 | | QSSTELLFGGH | | 17: | | | | | | | |
| 195 | NM_ | NP_001275537.1: | 42883 | SCGPGTQHRQLQCRQ | 957 | HLA- | 46.2 | 1.00E−04 | 3.79 | 3 | No | N/A | No |
| | 001288608.1: | p.Gly780 | | EFGGGGSSVPPERCG | | A*03: | | | | | | | |
| | c.2339del | ValfsTer62 | | HLPRPNITQS | | 26 | | | | | | | |
| 196 | NM_021191.2: | NP_067014.2: | 43792 | SSSLSSGHVHSTPFQA | 958 | HLA- | 121 | 1.00E−04 | 0.45 | 3 | No | N/A | No |
| | c.910del | p.Arg304Valfs | | GTPRYDVPIDMSYDS | | B*15: | | | | | | | |
| | | Ter6 | | YPHHGIGTQ | | 17 | | | | | | | |
| 197 | NM_003501.2: | NP_003492.2: | 43832 | ASTVEGGDTALLPEFP | 959 | HLA- | 11.5 | 1.00E−04 | 1.04 | 3 | No | N/A | No |
| | c.61del | p.Leu21Serfs | | RGPLDAYRARASFSW | | B*15: | | | | | | | |
| | | Ter57 | | KELALFTEG | | 17 | | | | | | | |
| 198 | NM_003500.3: | NP_003491.1: | 45421 | ARRGMHAFIVPIRSLQ | 960 | HLA- | 32.3 | 1.00E−04 | 0.4 | 3 | No | N/A | No |
| | c.695del | p.Pro232Hisfs | | DHTPLPGIIIGDIGPKM | | B*15: | | | | | | | |
| | | Ter26 | | DFDQTDN | | 17 | | | | | | | |
| 199 | NM_001420.3: | NP_001411.2: | 45496 | MVTQILGAMESQVG | 961 | HLA- | 85.1 | 1.00E−04 | 0.54 | 3 | No | N/A | yes |
| | c.47del | p.Gly16Alafs | | GGPAGPALPNGPLLG | | B*15: | | | | | | | |
| | | Ter35 | | TNGATDD | | 17 | | | | | | | |

SUPPLEMENTARY TABLE 6
List of the top 100 most immunogenic predicted MHC-II neoAgs obtained from the computational methods in the discovery set.

| | | | | | | Micro- | Reference | | Altered | Number | |
|---|---|---|---|---|---|---|---|---|---|---|---|
| Mutant | SEQ | | | | | satel- | MS | | MS | deleted | Pep- |
| Epitope | ID | Gene | Chromo- | | | lite | Lengths | Variant | Length | nucleo- | tide |
| Sequence | NO | Name | some | Start | Stop | motif | (repeats) | Tpe | (repeats) | tides | Length |
| RWMVLRNSWRAVARM | 218 | P4HB | chr17 | 79803763 | 79803764 | T | 8 | FS | 7 | −1 | 15 |
| IDNIKRNHNLALGRQ | 219 | RAD50 | chr5 | 131931451 | 131931452 | A | 9 | FS | 8 | −1 | 15 |
| PKMQVTITLTSPIIR | 220 | ZFR | chr5 | 32404160 | 32404161 | A | 9 | FS | 8 | −1 | 15 |
| FQVHFLKSGGLPLVL | 221 | USP9Y | chrY | 14847610 | 14847611 | T | 7 | FS | 6 | −1 | 15 |
| KTGLQLLRNHIEELK | 222 | GOLIM4 | chr3 | 167728580 | 167728581 | A | 7 | FS | 6 | −1 | 15 |
| QKKLMLLRLNLRKMC | 223 | SEC31A | chr4 | 83785564 | 83785565 | T | 9 | FS | 8 | −1 | 15 |
| LINIHHRKNPLLPMR | 224 | KMT2C | chr7 | 151874147 | 151874148 | A | 9 | FS | 8 | −1 | 15 |
| WILHLLGLRPPSLLS | 225 | NTAN1 | chr16 | 15131989 | 15131990 | A | 7 | FS | 6 | −1 | 15 |
| LKETKFITYRSKKLI | 226 | TCERG1 | chr5 | 145886730 | 145886731 | A | 8 | FS | 7 | −1 | 15 |
| EDIEFHFSLGWTMLV | 227 | MFN2 | chr1 | 12052735 | 12052736 | T | 7 | FS | 6 | −1 | 15 |
| GKNGFLQSRSSSLFS | 228 | GPBP1L1 | chr1 | 46120889 | 46120890 | T | 7 | FS | 6 | −1 | 15 |
| PALLLAEATHKASAL | 229 | TCF7L2 | chr10 | 114925316 | 114925317 | A | 9 | FS | 8 | −1 | 15 |
| SLDNVLRTMLRRFAR | 230 | SLC4A11 | chr20 | 3215424 | 3215425 | A | 7 | FS | 8 | −1 | 15 |
| YLRFIKSLAERTMSV | 231 | TET2 | chr4 | 106158293 | 106158298 | TAGAC | 2 | FS | 1 | −4 | 15 |
| KKDFGKMTANSVSVA | 232 | FAM111B | chr11 | 58892376 | 58892377 | A | 10 | FS | 9 | −1 | 15 |
| RHVIKVLLGRKVNWH | 233 | UBR5 | chr8 | 103289348 | 103289349 | A | 8 | FS | 7 | −1 | 15 |
| QLARFFPITPPVWHI | 234 | RNF43 | chr17 | 56435160 | 56435161 | G | 7 | FS | 6 | −1 | 15 |
| NAILLFLRTRGVCSV | 235 | BTN3A3 | chr6 | 26451946 | 26451947 | T | 6 | FS | 5 | −1 | 15 |
| FEEIIKNDGALLKKK | 236 | VPS13A | chr9 | 79931168 | 79931169 | A | 6 | FS | 5 | −1 | 15 |
| LRLLSLYRPPLAPLL | 237 | CIC | chr19 | 42799097 | 42799098 | C | 5 | FS | 4 | −1 | 15 |
| TKFITYRSKKLIQES | 238 | TCERG1 | chr5 | 145887464 | 145887465 | A | 8 | FS | 7 | −1 | 15 |
| LEVMLLNMGYRITGL | 239 | WDTC1 | chr1 | 27621107 | 27621108 | G | 8 | FS | 7 | −1 | 15 |
| SINVLCVRASLIEKL | 240 | SPINK5 | chr5 | 147499874 | 147499875 | A | 10 | FS | 9 | −1 | 15 |
| DDVLRNLKNFLLMKR | 241 | ERBIN | chr5 | 65342358 | 65342359 | T | 5 | FS | 4 | −1 | 15 |
| LGKLEMVKAVQLRVA | 242 | MCPH1 | chr8 | 6302638 | 6302639 | A | 7 | FS | 6 | −1 | 15 |
| TARISVNSNNVQSLL | 243 | KLHL7 | chr7 | 23163475 | 23163476 | T | 7 | FS | 6 | −1 | 15 |
| SPMALLLAARQRAQK | 244 | C6orf132 | chr6 | 42074305 | 42074306 | C | 7 | FS | 6 | −1 | 15 |
| GKVIMPLGSKLTGVI | 245 | BODIL1 | chr4 | 13610188 | 13610189 | A | 8 | FS | 7 | −1 | 15 |
| KKVRVIYTQLSKTVV | 246 | IKBKB | chr8 | 42176139 | 42176140 | NA | NA | FS | NA | −1 | 15 |
| AFFMNLTREPSRVLK | 247 | VPS13A | chr9 | 79984307 | 79984308 | T | 7 | FS | 6 | −1 | 15 |
| PSKRSLLSVGNLIGL | 248 | BTBD7 | chr14 | 93761192 | 93761193 | T | 8 | FS | 7 | −1 | 15 |
| PLPTALRQLRGRPAD | 249 | AXIN2 | chr17 | 63533938 | 63533940 | AG | 5 | FS | 4 | −1 | 15 |
| TVEMRRWWTLVMEWK | 250 | TCF20 | chr22 | 42564715 | 42564716 | G | 7 | FS | 6 | −1 | 15 |
| NKKMLTALPPAMTAM | 251 | WDR59 | chr16 | 74976690 | 74976691 | A | 8 | FS | 7 | −1 | 15 |
| KKKRQINRRKLQRKK | 252 | UPF3A | chr13 | 115057210 | 115057211 | A | 9 | FS | 8 | −1 | 15 |
| LKMVWRINPAHRKLQ | 253 | DYNC1H1 | chr14 | 102445787 | 102445788 | T | 7 | FS | 6 | −1 | 15 |
| PHRLRSLPRPLHLRL | 254 | KMT2D | chr12 | 49445525 | 49445526 | C | 7 | FS | 6 | −1 | 15 |
| NQNLYLVGASKIRMI | 255 | ANO10 | chr3 | 43647212 | 43647213 | A | 9 | FS | 8 | −1 | 15 |
| FHPYRRYPPPAAAAL | 256 | DAZAP1 | chr19 | 1434835 | 1434836 | C | 6 | FS | 5 | −1 | 15 |
| ESNLLQSPSSILSTL | 257 | PTTG1 | chr5 | 159854836 | 159854837 | C | 6 | FS | 5 | −1 | 15 |
| LWGVRMTSLSASTSL | 258 | FLCN | chr17 | 17119708 | 17119709 | C | 8 | FS | 7 | −1 | 15 |
| LSSLVKKILAMTLTL | 259 | DNAH7 | chr2 | 196788373 | 196788374 | A | 9 | FS | 8 | −1 | 15 |
| KKTVLSLVTISRFVL | 260 | RABGAP1 | chr9 | 125861041 | 125861042 | A | 8 | FS | 7 | −1 | 15 |
| NSVIVGNTHGQLAEI | 261 | WDR74 | chr11 | 62603470 | 62603472 | AG | 2 | FS | 1 | −1 | 15 |
| QLPVYKLLPSQNRLQ | 262 | APC | chr5 | 112174833 | 112174834 | A | 4 | FS | 3 | −1 | 15 |
| IRKGFQLRKTARGRG | 263 | INF2 | chr14 | 105174184 | 105174185 | C | 7 | FS | 6 | −1 | 15 |
| GVFISKVLPRGLAAR | 264 | SCRIB | chr8 | 144886851 | 144886852 | C | 6 | FS | 5 | −1 | 15 |
| QRRLIKSMESVMVKY | 265 | POLR2A | chr17 | 7388097 | 7388098 | C | 7 | FS | 6 | −1 | 15 |
| KKNILNSLPSSMEIA | 266 | TBC1D23 | chr3 | 100039735 | 100039736 | A | 9 | FS | 8 | −1 | 15 |
| NRVFKLAPNLTELRA | 267 | PLXNA3 | chrX | 153688564 | 153688565 | G | 8 | FS | 7 | −1 | 15 |
| KLICQMTRTNRLFGM | 268 | SEC16A | chr9 | 139345822 | 139345823 | C | 7 | FS | 6 | −1 | 15 |
| NSKLRYKKRGVIAWR | 269 | MYO1A | chr12 | 57422572 | 57422573 | A | 8 | FS | 7 | −1 | 15 |
| SFASMGMLEARIRIL | 270 | CTSC | chr11 | 88068107 | 88068108 | T | 6 | FS | 5 | −1 | 15 |
| DARLRASTALLLPIL | 271 | FAM179B | chr14 | 45432121 | 45432122 | G | 5 | FS | 4 | −1 | 15 |
| ARLCLIVSRTLLLVQ | 272 | MSH3 | chr5 | 79970914 | 79970915 | A | 8 | FS | 7 | −1 | 15 |
| ALSVLTASLSYMVGM | 273 | GRINA | chr8 | 145065717 | 145065718 | C | 7 | FS | 6 | −1 | 15 |
| AWFIRESMTIYIFCL | 274 | UGCG | chr9 | 114695179 | 114695180 | T | 7 | FS | 6 | −1 | 15 |
| ERLLFFAVPPQILAS | 275 | CNTROB | chr17 | 7849144 | 7849145 | NA | NA | FS | NA | −1 | 15 |
| MFFMVFLIIWQNTME | 276 | CASP5 | chr11 | 104878040 | 104878041 | A | 10 | FS | 9 | −1 | 15 |
| VFFAYLVAHSFLSVF | 277 | SLC44A3 | chr1 | 95357931 | 95357932 | T | 7 | FS | 6 | −1 | 15 |
| GTGASMASIMETIGL | 278 | C6orf132 | chr6 | 42110042 | 42110043 | G | 5 | FS | 4 | −1 | 15 |
| LLPYPFHVLALEVTF | 279 | WNK4 | chr17 | 40939869 | 40939870 | G | 7 | FS | 6 | −1 | 15 |
| PLRICVTLWSRLVLA | 280 | RAB3GAP2 | chr1 | 220355681 | 220355682 | T | 7 | FS | 6 | −1 | 15 |
| ERVQTVAASTMRVAV | 281 | RERE | chr1 | 8421827 | 8421828 | A | 5 | FS | 4 | −1 | 15 |
| YQYTVFLRSDSYMGL | 282 | SEC63 | chr6 | 108214754 | 108214755 | A | 10 | FS | 9 | −1 | 15 |
| QRYRSVLRGWWILLT | 283 | RNF186 | chr1 | 20141313 | 20141314 | G | 5 | FS | 4 | −1 | 15 |
| LYGWYQLCVSSMKLL | 284 | USP24 | chr1 | 55619561 | 55619562 | A | 7 | FS | 6 | −1 | 15 |
| TGATCGKRAARLVLR | 285 | BCORL1 | chrX | 129149049 | 129149050 | A | 5 | FS | 4 | −1 | 15 |
| LSDIYLNNVIMRFMQ | 286 | SRGAP1 | chr12 | 64377820 | 64377821 | A | 7 | FS | 6 | −1 | 15 |
| RLFLLQDSGRILQLL | 287 | WDR6 | chr3 | 49051381 | 49051382 | G | 7 | FS | 6 | −1 | 15 |
| APQVTRLRSLNHLLI | 288 | SPAG9 | chr17 | 49077040 | 49077041 | A | 9 | FS | 8 | −1 | 15 |
| GIFL VIETHGMAVSW | 289 | MUC5B | chr11 | 1250517 | 1250518 | C | 6 | FS | 5 | −1 | 15 |
| ANRYFSPNFKVKLYF | 290 | PTEN | chr10 | 89720811 | 89720812 | A | 6 | FS | 5 | −1 | 15 |
| WRLFLIIQTTGYQSI | 291 | SMC3 | chr10 | 112333493 | 112333494 | T | 7 | FS | 6 | −1 | 15 |
| GSQSIMMSWMPPLAP | 292 | PLEKHA6 | chr1 | 204228410 | 204228411 | G | 6 | FS | 5 | −1 | 15 |
| GNLLSFSRRGMKSSV | 293 | FAHD2A | chr2 | 96078465 | 96078466 | C | 7 | FS | 6 | −1 | 15 |
| HLTLARMKHFIYFKH | 294 | TRPM7 | chr15 | 50925139 | 50925140 | A | 7 | FS | 6 | −1 | 15 |
| YTIFYRTIIGNETAV | 295 | IL6ST | chr5 | 55247868 | 55247869 | A | 7 | FS | 6 | −1 | 15 |
| WKVKLPSSMSVALPL | 296 | MFSD5 | chr12 | 53646697 | 53646698 | A | 3 | FS | 2 | −1 | 15 |
| FQELILNQASMAPPR | 297 | ATAD2B | chr2 | 24086325 | 24086326 | T | 7 | FS | 6 | −1 | 15 |
| KERLFRNFGGLLGPL | 298 | XYLT2 | chr17 | 48433966 | 48433967 | C | 7 | FS | 6 | −1 | 15 |
| YSKVRALGGVNAARR | 299 | CLCA1 | chr1 | 86961263 | 86961264 | A | 4 | FS | 3 | −1 | 15 |
| MMIVLTIQNAAFLSN | 300 | ZMYM2 | chr13 | 20638676 | 20638677 | A | 8 | FS | 7 | −1 | 15 |
| LIWIVFISSGHVASA | 301 | CREBBP | chr16 | 3789590 | 3789591 | C | 6 | FS | 5 | −1 | 15 |
| EPFIQKDVELRIMPP | 302 | APC | chr5 | 112175211 | 112175216 | AAAGA | 2 | FS | 1 | −4 | 15 |
| GRLQIMSLENLSIEK | 303 | ASNSD1 | chr2 | 190535352 | 190535353 | A | 5 | FS | 4 | −1 | 15 |
| WPITELKIQMRGILG | 304 | GOLGA3 | chr12 | 133384945 | 133384946 | A | 7 | FS | 6 | −1 | 15 |
| RELLKTLNMIQVLMS | 305 | SMARCAD1 | chr4 | 95173909 | 95173910 | A | 8 | FS | 7 | −1 | 15 |
| SAFSSLLPLRNLSQL | 306 | RNF25 | chr2 | 219529513 | 219529514 | G | 7 | FS | 6 | −1 | 15 |
| ILGSFFMATSSHRFL | 307 | SLC17A5 | chr6 | 74351589 | 74351590 | A | 7 | FS | 6 | −1 | 15 |
| RSRLMRQSRRSTQGV | 308 | PPP1R12C | chr19 | 55607461 | 55607462 | G | 6 | FS | 5 | −1 | 15 |
| PSWPMAVPLAASRAS | 309 | GIPC1 | chr19 | 14593639 | 14593640 | A | 14 | FS | 13 | −1 | 15 |
| KLQAVLEDTLLKILL | 310 | CCDC186 | chr10 | 115885657 | 115885658 | A | 6 | FS | 5 | −1 | 15 |
| KSLVRLSSCVPVALM | 311 | TGFBR2 | chr3 | 30691871 | 30691872 | A | 10 | FS | 9 | −1 | 15 |
| ACRLRWARPEPAAQA | 312 | PRDM2 | chr1 | 14108748 | 14108749 | A | 9 | FS | 8 | −1 | 15 |
| IKICILWNQIMHASW | 313 | CACNA2D1 | chr7 | 82072734 | 82072735 | T | 4 | FS | 3 | −1 | 15 |
| HVPLSISGSPALELL | 314 | CNOT9 | chr2 | 219449363 | 219449364 | T | 7 | FS | 6 | −1 | 15 |
| HKKLILEKSPINVKK | 315 | ZNF124 | chr1 | 247319908 | 247319909 | A | 7 | FS | 6 | −1 | 15 |
| EKDLMQLAQATAVAA | 316 | BPTF | chr17 | 65944265 | 65944266 | A | 7 | FS | 6 | −1 | 15 |
| FTIYSISSLKTLFRK | 317 | ALG8 | chr11 | 77832192 | 77832193 | A | 7 | FS | 6 | −1 | 15 |

| | | | | | | | | | | | | Pre- |
|---|---|---|---|---|---|---|---|---|---|---|---|---|
| | | | | | | | | | | | | dicted |
| | | | | | | | Bind- | | | | | in |
| | | | Immuno- | Wild- | | | ing | Tumor | | | Eli- | the |
| SEQ | | | genic- | type | SEQ | | Affin- | Abun- | Sample | Eli- | spot | valida- |
| ID | | | ity | se- | ID | HLA | ity | dance | Recur- | spot | reac- | tion |
| NO | HGVSc | HGVSp | Score | quence | NO | Allele | (nM) | (TPM) | rence | tested | tive | set |
| 218 | NM_000918.3: | NP_000909.2: | 1 | PVKVLV | 783 | HLA-DRB1* | 14.23 | 368.51 | 2 | Tested | No | No |
| | c.1160del | p.Asn387Thr | | GKNFED | | 13: | | | | | | |
| | | fsTer118 | | VAFDEK | | 01 | | | | | | |
| | | | | KNVFVE | | | | | | | | |
| | | | | FYAPWC | | | | | | | | |
| | | | | GHCKQL | | | | | | | | |
| | | | | APIW | | | | | | | | |
| 219 | NM_005732.3: | NP_005723.2: | 10 | SKLRLA | 780 | HLA-DRB1* | 14.55 | 39.05 | 2 | Tested | No | No |
| | c.2165del | p.Lys722Arg | | PDKLKS | | 13: | | | | | | |
| | | fsTer14 | | TESELK | | 02 | | | | | | |
| | | | | KKEKRR | | | | | | | | |
| | | | | DEMLGL | | | | | | | | |
| | | | | VPMRQS | | | | | | | | |
| | | | | IIDL | | | | | | | | |
| 220 | NM_016107.3: | NP_057191.2: | 14 | CAGPQT | 785 | HLA-DRB1* | 24.78 | 51.75 | 2 | Tested | No | No |
| | c.1074del | p.Glu359Lys | | YKEHLE | | 07: | | | | | | |
| | | fsTer4 | | GQKHKK | | 01 | | | | | | |
| | | | | KEAALK | | | | | | | | |
| | | | | ASQNTS | | | | | | | | |
| | | | | SSNSST | | | | | | | | |
| | | | | RGTQ | | | | | | | | |
| 221 | NM_004654.3: | NP_004645.2: | 15 | DLINKF | 782 | HLA-DRB1* | 9.4 | 30.44 | 1 | Tested | Yes | No |
| | c.729del | p.Phe243Leu | | GTLNGF | | 07: | | | | | | |
| | | fsTer6 | | QILHDR | | 01 | | | | | | |
| | | | | FENGSA | | | | | | | | |
| | | | | LNIQII | | | | | | | | |
| | | | | AALIKP | | | | | | | | |
| | | | | FGQC | | | | | | | | |
| 222 | NM_014498.4: | NP_055313.1: | 30 | QYQEEA | 778 | HLA-DRB1* | 57.67 | 216.91 | 2 | Tested | No | No |
| | c.1891del | p.Arg631Gly | | EEEVQE | | 13: | | | | | | |
| | | fsTer87 | | DLTEEK | | 02 | | | | | | |
| | | | | KRELEH | | | | | | | | |
| | | | | NAEETY | | | | | | | | |
| | | | | GENDEN | | | | | | | | |
| | | | | TDDK | | | | | | | | |
| 223 | NM_001318120.1: | NP_001305049.1: | 61 | DQLQQA | 789 | HLA-DRB1* | 14.7 | 24.08 | 3 | Tested | No | yes |
| | c.1384del | p.Ile462Leu | | VQSQGF | | 13: | | | | | | |
| | | fsTer16 | | INYCQK | | 01 | | | | | | |
| | | | | KIDASQ | | | | | | | | |
| | | | | TEFEKN | | | | | | | | |
| | | | | VWSFLK | | | | | | | | |
| | | | | VNFE | | | | | | | | |
| 224 | NM_170606.2: | NP_733751.2: | 167 | DLPIDD | 786 | HLA-DRB1* | 99.76 | 52.26 | 2 | Tested | No | No |
| | c.8390del | p.Lys2797Arg | | KLDNQC | | 13: | | | | | | |
| | | fsTer26 | | VSVEPK | | 02 | | | | | | |
| | | | | KKEQEN | | | | | | | | |
| | | | | KTLVLS | | | | | | | | |
| | | | | DKHSPQ | | | | | | | | |
| | | | | KKST | | | | | | | | |
| 225 | NM_173474.3: | NP_775745.1: | 173 | LAEPPH | 790 | HLA-DRB1* | 46.89 | 27.57 | 1 | No | N/A | No |
| | c.831del | p.Lys277Asn | | FVEHIR | | 15: | | | | | | |
| | | fsTer83 | | STLMFL | | 01 | | | | | | |
| | | | | KKHPSP | | | | | | | | |
| | | | | AHTLFS | | | | | | | | |
| | | | | GNKALL | | | | | | | | |
| | | | | YKKN | | | | | | | | |
| 226 | NM_006706.3: | NP_006697.2: | 189 | REEKEK | 849 | HLA-DRB1* | 43.32 | 83.94 | 1 | No | N/A | No |
| | c.2871del | p.Arg958Glu | | LFNEHI | | 07: | | | | | | |
| | | fsTer16 | | EALTKK | | 01 | | | | | | |
| | | | | KREHFR | | | | | | | | |
| | | | | QLLDET | | | | | | | | |
| | | | | SAITLT | | | | | | | | |
| | | | | STWK | | | | | | | | |
| 227 | NM_001127660.1: | NP_001121132.1: | 190 | SKVRGI | 794 | HLA-DRB1* | 14.83 | 35.57 | 2 | Tested | No | No |
| | c.306del | p.Phe102Leu | | SEVLAR | | 07: | | | | | | |
| | | fsTer11 | | RHMKVA | | 01 | | | | | | |
| | | | | FFGRTS | | | | | | | | |
| | | | | NGKSTV | | | | | | | | |
| | | | | INAMLW | | | | | | | | |
| | | | | DKVL | | | | | | | | |
| 228 | NM_021639.4: | NP_067652.1: | 247 | RGEGRF | 781 | HLA-DRB1* | 86.74 | 64.4 | 1 | No | N/A | No |
| | c.162del | p.Phe54Leu | | GVSRRR | | 04: | | | | | | |
| | | fsTer53 | | HNSSDG | | 05 | | | | | | |
| | | | | FFNNGP | | | | | | | | |
| | | | | LRTAGD | | | | | | | | |
| | | | | SWHQPS | | | | | | | | |
| | | | | LFRH | | | | | | | | |
| 229 | NM_001146274.1: | NP_001139746.1: | 263 | ALFGLD | 788 | HLA-DRB1* | 59.39 | 44.54 | 5 | Tested | No | No |
| | c.1403del | p.Lys468Ser | | RQTLWC | | 13: | | | | | | |
| | | fsTer23 | | KPCRRK | | 01 | | | | | | |
| | | | | KKCVRY | | | | | | | | |
| | | | | IQGEGS | | | | | | | | |
| | | | | CLSPPS | | | | | | | | |
| | | | | SDGS | | | | | | | | |
| 230 | NM_001174090.1: | NP_001167561.1: | 288 | DEAFDT | 801 | HLA-DRB1* | 12.84 | 25.71 | 2 | Tested | No | No |
| | c.333del | p.Phe111Leu | | ANSSIV | | 13: | | | | | | |
| | | fsTer32 | | SGESIR | | 01 | | | | | | |
| | | | | FFVNVN | | | | | | | | |
| | | | | LEMQAT | | | | | | | | |
| | | | | NTENEA | | | | | | | | |
| | | | | TSGG | | | | | | | | |
| 231 | NM_001127208.2: | NP_001120680.1: | 301 | LKSQKQ | 800 | HLA-DRB1* | 20.47 | 51.55 | 2 | Tested | No | No |
| | c.3198_3202del | p.Arg1067Asn | | VKVEMS | | 07: | | | | | | |
| | | fsTer7 | | GPVTVL | | 01 | | | | | | |
| | | | | TRQTTA | | | | | | | | |
| | | | | AELDSH | | | | | | | | |
| | | | | TPALEQ | | | | | | | | |
| | | | | QTTS | | | | | | | | |
| 232 | NM_1989473: | NP_945185.1: | 350 | SMVDEV | 815 | HLA-DRB1* | 16.04 | 10.65 | 1 | No | N/A | No |
| | c.816del | p.Ala273His | | SGKVLE | | 10: | | | | | | |
| | | fsTer26 | | MDISKK | | 01 | | | | | | |
| | | | | KALQQK | | | | | | | | |
| | | | | DIHKKI | | | | | | | | |
| | | | | KQNESA | | | | | | | | |
| | | | | TDEI | | | | | | | | |
| 233 | NM_015902.5: | NP_056986.2: | 375 | MSYAAN | 797 | HLA-DRB1* | 36.66 | 61.73 | 4 | Tested | No | No |
| | c.6360del | p.Glu2121Lys | | LKNVMN | | 15: | | | | | | |
| | | fsTer28 | | MQNRQK | | 01 | | | | | | |
| | | | | KEGEEQ | | | | | | | | |
| | | | | PVLPEE | | | | | | | | |
| | | | | TESSKP | | | | | | | | |
| | | | | GPSA | | | | | | | | |
| 234 | NM_017763.4: | NP_060233.3: | 381 | FNLQKS | 784 | HLA-DRB1* | 109.73 | 79.41 | 8 | Tested | No | yes |
| | c.1976del | p.Gly659Val | | SLSARH | | 04: | | | | | | |
| | | fsTer41 | | PQRKRR | | 05 | | | | | | |
| | | | | GGPSEP | | | | | | | | |
| | | | | TPGSRP | | | | | | | | |
| | | | | QDATVH | | | | | | | | |
| | | | | PACQ | | | | | | | | |
| 235 | NM_006994.4: | NP_008925.1: | 388 | LFKPAD | 814 | HLA-DRB1* | 32.99 | 101.56 | 1 | No | N/A | No |
| | c.1063del | p.Val355Phe | | VILDPD | | 15: | | | | | | |
| | | fsTer71 | | TANAIL | | 01 | | | | | | |
| | | | | LVSEDQ | | | | | | | | |
| | | | | RSVQRA | | | | | | | | |
| | | | | EEPRDL | | | | | | | | |
| | | | | PDNP | | | | | | | | |
| 236 | NM_033305.2: | NP_150648.2: | 442 | EINVII | 803 | HLA-DRB1* | 24.8 | 189.72 | 2 | Tested | No | No |
| | c.4715del | p.Asn1572Met | | KNPEIV | | 03: | | | | | | |
| | | fsTer6 | | FVADMT | | 01 | | | | | | |
| | | | | KNDAPA | | | | | | | | |
| | | | | LVITTQC | | | | | | | | |
| | | | | EICYKG | | | | | | | | |
| | | | | NLE | | | | | | | | |
| 237 | NM_001304815.1: | NP_001291744.1: | 456 | KIREVR | 802 | HLA-DRB1* | 24.2 | 24.77 | 1 | No | N/A | No |
| | c.7313del | p.Pro2438Leu | | QKIMQA | | 15: | | | | | | |
| | | fsTer91 | | ATPTEQ | | 01 | | | | | | |
| | | | | PPGAEA | | | | | | | | |
| | | | | PLPVPP | | | | | | | | |
| | | | | PTGTAA | | | | | | | | |
| | | | | APAP | | | | | | | | |
| 238 | NM_006706.3: | NP_006697.2: | 461 | LLDETS | 828 | HLA-DRB1* | 36.16 | 92.56 | 2 | Tested | No | No |
| | c.2947del | p.Ile983Ser | | AITLTS | | 13: | | | | | | |
| | | fsTer41 | | TWKEVK | | 01 | | | | | | |
| | | | | KIIKED | | | | | | | | |
| | | | | PRCIKF | | | | | | | | |
| | | | | SSSDRK | | | | | | | | |
| | | | | KQRE | | | | | | | | |
| 239 | NM_001276252.1: | NP_001263181.1: | 476 | ATYVTF | 804 | HLA-DRB1* | 30.05 | 10.01 | 6 | Tested | No | No |
| | c.868del | p.Glu290Asn | | SPNGTE | | 15: | | | | | | |
| | | fsTer8 | | LLVNMG | | 01 | | | | | | |
| | | | | GEQVYL | | | | | | | | |
| | | | | FDLTYK | | | | | | | | |
| | | | | QRPYTF | | | | | | | | |
| | | | | LLPR | | | | | | | | |
| 240 | NM_001127698.1: | NP_001121170.1: | 499 | GNKCTM | 779 | HLA-DRB1* | 123.19 | 461.65 | 3 | Tested | No | yes |
| | c.2468del | p.Lys823Arg | | CKEKLE | | 13: | | | | | | |
| | | fsTer119 | | REAAEK | | 02 | | | | | | |
| | | | | KKKEDE | | | | | | | | |
| | | | | DRSNTG | | | | | | | | |
| | | | | ERSNTG | | | | | | | | |
| | | | | ERSN | | | | | | | | |
| 241 | NM_001253699.1: | NP_001240628.1: | 505 | TANMKA | 812 | HLA-DRB1* | 27.41 | 71.74 | 1 | No | N/A | No |
| | c.1785del | p.Phe595Leu | | SENLKH | | 15: | | | | | | |
| | | fsTer13 | | IVNHDD | | 01 | | | | | | |
| | | | | VFEESE | | | | | | | | |
| | | | | ELSSDE | | | | | | | | |
| | | | | EMKMAE | | | | | | | | |
| | | | | MRPP | | | | | | | | |
| 242 | NM_001322042.1: | NP_001308971.1: | 515 | KKERTS | 981 | HLA-DRB1* | 18.8 | 24.36 | 1 | No | N/A | No |
| | c.1402del | p.Thr468Pro | | IFEMSD | | 07: | | | | | | |
| | | fsTer32 | | FSCVGK | | 01 | | | | | | |
| | | | | KTRTVD | | | | | | | | |
| | | | | ITNFTA | | | | | | | | |
| | | | | KTISSP | | | | | | | | |
| | | | | RKTG | | | | | | | | |
| 243 | NM_001031710.2: | NP_001026880.2: | 561 | VQERKI | 816 | HLA-DRB1* | 17.34 | 13.38 | 2 | Tested | No | No |
| | c.207del | p.Phe69Leu | | PAHRVV | | 13: | | | | | | |
| | | fsTer3 | | LAAASH | | 02 | | | | | | |
| | | | | FFNLMF | | | | | | | | |
| | | | | TTNMLE | | | | | | | | |
| | | | | SKSFEV | | | | | | | | |
| | | | | ELKD | | | | | | | | |
| 244 | NM_001164446.1: | NP_001157918.1: | 585 | FTKTPK | 824 | HLA-DRB1* | 19.6 | 9.67 | 1 | No | N/A | No |
| | c.1344del | p.Ser449Ala | | SSSPAL | | 13: | | | | | | |
| | | fsTer68 | | KPKPNP | | 01 | | | | | | |
| | | | | PSPENT | | | | | | | | |
| | | | | ASSAPV | | | | | | | | |
| | | | | DWRDPS | | | | | | | | |
| | | | | QMEK | | | | | | | | |
| 245 | NM_148894.2: | NP_683692.2: | 660 | PKAARI | 808 | HLA-DRB1* | 37.15 | 45.9 | 1 | No | N/A | No |
| | c.1707del | p.Val570Ter | | KEVLKE | | 15: | | | | | | |
| | | | | RKVLEK | | 01 | | | | | | |
| | | | | KVALSK | | | | | | | | |
| | | | | KRKKDS | | | | | | | | |
| | | | | RNVEEN | | | | | | | | |
| | | | | SKKK | | | | | | | | |
| 246 | NM_001556.2: | NP_001547.1: | 692 | RNLAFF | 807 | HLA-DRB1* | 49.54 | 62.92 | 1 | No | N/A | No |
| | c.1312del | p.Gln438Arg | | QLRKVW | | 15: | | | | | | |
| | | fsTer3 | | GQVWHS | | 01 | | | | | | |
| | | | | IQTLKE | | | | | | | | |
| | | | | DCNRLQ | | | | | | | | |
| | | | | QGQRAA | | | | | | | | |
| | | | | MMNL | | | | | | | | |
| 247 | NM_033305.2: | NP_150648.2: | 731 | LGNPFG | 982 | HLA-DRB1* | 128.34 | 186.75 | 1 | No | N/A | No |
| | c.8653del | p.Tyr2885Met | | LIREFS | | 07: | | | | | | |
| | | fsTer20 | | EGVEAF | | 01 | | | | | | |
| | | | | FYEPYQ | | | | | | | | |
| | | | | GAIQGP | | | | | | | | |
| | | | | EEFVEG | | | | | | | | |
| | | | | MALG | | | | | | | | |
| 248 | NM_001002860.2: | NP_001002860.2: | 734 | CESKLY | 876 | HLA-DRB1* | 48.05 | 30.99 | 2 | Tested | No | No |
| | c.173del | p.Lys58Arg | | SLDHGH | | 07: | | | | | | |
| | | fsTer44 | | EKPQDK | | 01 | | | | | | |
| | | | | KKRTSG | | | | | | | | |
| | | | | LATLKK | | | | | | | | |
| | | | | KFIKRR | | | | | | | | |
| | | | | KSNR | | | | | | | | |
| 249 | NM_004655.3: | NP_004646.3: | 780 | ESRHSL | 983 | HLA-DRB1* | 105.99 | 67.54 | 1 | No | N/A | No |
| | c.1214_1215del | p.Glu405Gly | | EERLOQ | | 13: | | | | | | |
| | | fsTer56 | | IREDEE | | 01 | | | | | | |
| | | | | REGSEL | | | | | | | | |
| | | | | TLNSRE | | | | | | | | |
| | | | | GAPTQH | | | | | | | | |
| | | | | PLSL | | | | | | | | |
| 250 | NM_005650.2: | NP_005641.1: | 785 | NFSVRC | 787 | HLA-DRB1* | 101.72 | 39.27 | 5 | Tested | No | No |
| | c.5826del | p.Leu1943Cys | | PKHKPP | | 15: | | | | | | |
| | | fsTer118 | | LPCPLP | | 01 | | | | | | |
| | | | | PLQNKT | | | | | | | | |
| | | | | AKGSLS | | | | | | | | |
| | | | | TEQSER | | | | | | | | |
| | | | | G | | | | | | | | |
| 251 | NM_030581.3: | NP_085058.3: | 792 | PTVALS | 792 | HLA-DRB1* | 79.51 | 39.18 | 2 | Tested | No | No |
| | c.479del | p.Asn160Met | | AVAGAS | | 07: | | | | | | |
| | | fsTer28 | | QVKWNK | | 01 | | | | | | |
| | | | | KNANCL | | | | | | | | |
| | | | | ATSHDG | | | | | | | | |
| | | | | DVRIWD | | | | | | | | |
| | | | | KRKP | | | | | | | | |
| 252 | NM_023011.3: | NP_075387.1: | 793 | RLREEE | 984 | HLA-DRB1* | 58.22 | 23.32 | 1 | No | N/A | No |
| | c.798del | p.Glu267Arg | | KRRRRE | | 13: | | | | | | |
| | | fsTer13 | | EERCKK | | 01 | | | | | | |
| | | | | KETDKQ | | | | | | | | |
| | | | | KKIAEK | | | | | | | | |
| | | | | EVRIKL | | | | | | | | |
| | | | | LKKP | | | | | | | | |
| 253 | NM_001376.4: | NP_001367.2: | 803 | EDSPYE | 819 | HLA-DRB1* | 11.9 | 143.82 | 1 | No | N/A | No |
| | c.483del | p.Phe161Leu | | TLHSFI | | 13: | | | | | | |
| | | fsTer52 | | SNAVAP | | 01 | | | | | | |
| | | | | FFKSYI | | | | | | | | |
| | | | | RESGKA | | | | | | | | |
| | | | | DRDGDK | | | | | | | | |
| | | | | MAPS | | | | | | | | |
| 254 | NM_003482.3: | NP_003473.3: | 848 | PPPEDS | 845 | HLA-DRB1* | 25.89 | 38.47 | 1 | No | N/A | No |
| | c.1940del | p.Pro647His | | PMSPPP | | 13: | | | | | | |
| | | fsTer283 | | EESPMS | | 01 | | | | | | |
| | | | | PPPEVS | | | | | | | | |
| | | | | RLSPLP | | | | | | | | |
| | | | | VVSRLS | | | | | | | | |
| | | | | PPPE | | | | | | | | |
| 255 | NM_001346464.1: | NP_001333393.1: | 892 | DVKEET | 827 | HLA-DRB1* | 15.83 | 33.46 | 2 | Tested | No | No |
| | c.132del | p.Asp45Met | | KEWLKN | | 07: | | | | | | |
| | | fsTer12 | | RIIAKK | | 01 | | | | | | |
| | | | | KDGGAQ | | | | | | | | |
| | | | | LLFRPL | | | | | | | | |
| | | | | LNKYEQ | | | | | | | | |
| | | | | ETLE | | | | | | | | |
| 256 | NM_018959.2: | NP_061832.2: | 936 | SQQPPS | 985 | HLA-DRB1* | 39.39 | 47.53 | 2 | Tested | No | No |
| | c.1155del | p.Ala386Pro | | YGGPSV | | 10: | | | | | | |
| | | fsTer43 | | PGSGGP | | 01 | | | | | | |
| | | | | PAGGSG | | | | | | | | |
| | | | | FGRGQN | | | | | | | | |
| | | | | HNVQGF | | | | | | | | |
| | | | | HPYR | | | | | | | | |
| 257 | NM_001282382.1: | NP_001269311.1: | 938 | PLMILD | 805 | HLA-DRB1* | 69.46 | 124.61 | 1 | No | N/A | No |
| | c.491del | p.Pro164Leu | | EERELE | | 13: | | | | | | |
| | | fsTer4 | | KLFQLG | | 02 | | | | | | |
| | | | | PPSPVK | | | | | | | | |
| | | | | MPSPPW | | | | | | | | |
| | | | | ESNLLQ | | | | | | | | |
| | | | | SPSS | | | | | | | | |
| 258 | NM_144997.5: | NP_659434.2: | 947 | EEAYRC | 796 | HLA-DQA1* | 118.96 | 60.55 | 1 | No | N/A | No |
| | c.1285del | p.His429Thr | | NFLGLS | | 01: | | | | | | |
| | | fsTer39 | | PHVQIP | | 02/DQB1* | | | | | | |
| | | | | PHVLSS | | 06: | | | | | | |
| | | | | EFAVIV | | 02 | | | | | | |
| | | | | EVHAAA | | | | | | | | |
| | | | | RSTL | | | | | | | | |
| 259 | NM_018897.2: | NP_061720.2: | 1113 | ALVVLD | 986 | HLA-DRB1* | 48.41 | 8.86 | 1 | No | N/A | No |
| | c.3770del | p.Asn1257Ile | | VHARDV | | 07: | | | | | | |
| | | fsTer11 | | LSSLVK | | 01 | | | | | | |
| | | | | KNISDD | | | | | | | | |
| | | | | SDFEWL | | | | | | | | |
| | | | | SQLRYY | | | | | | | | |
| | | | | WQEN | | | | | | | | |
| 260 | NM_012197.3: | NP_036329.3: | 1123 | QLKEMC | 872 | HLA-DRB1* | 69.8 | 39.43 | 2 | Tested | No | No |
| | c.2789del | p.Asn930Thr | | RRELDK | | 07: | | | | | | |
| | | fsTer15 | | AESEIK | | 01 | | | | | | |
| | | | | KNSSII | | | | | | | | |
| | | | | GDYKQI | | | | | | | | |
| | | | | CSQLSE | | | | | | | | |
| | | | | RLEK | | | | | | | | |
| 261 | NM_018093.3: | NP_060563.2: | 1133 | RGLAQA | 851 | HLA-DRB1* | 59.28 | 18.78 | 2 | Tested | No | No |
| | c.330_331del | p.Arg110Ser | | DGTLIT | | 13: | | | | | | |
| | | fsTer4 | | CVDSGI | | 02 | | | | | | |
| | | | | LRVWHD | | | | | | | | |
| | | | | KDKDTS | | | | | | | | |
| | | | | SDPLLE | | | | | | | | |
| | | | | LRVG | | | | | | | | |
| 262 | NM_000038.5: | NP_000029.2: | 1138 | SIKYNE | 852 | HLA-DRB1* | 15.64 | 36.82 | 1 | No | N/A | No |
| | c.3546del | p.Lys1182Asn | | EKRHVD | | 10: | | | | | | |
| | | fsTer83 | | QPIDYS | | 01 | | | | | | |
| | | | | LKYATD | | | | | | | | |
| | | | | IPSSQK | | | | | | | | |
| | | | | QSFSFS | | | | | | | | |
| | | | | KSSS | | | | | | | | |
| 263 | NM_022489.3: | NP_071934.3: | 1182 | GWGPPP | 820 | HLA-DRB1* | 36.29 | 109.29 | 1 | No | N/A | No |
| | c.1587del | p.Val530Trp | | PPPPLL | | 13: | | | | | | |
| | | fsTer28 | | PCTCSP | | 01 | | | | | | |
| | | | | PVAGGM | | | | | | | | |
| | | | | EEVIVA | | | | | | | | |
| | | | | QVDHGL | | | | | | | | |
| | | | | GSAW | | | | | | | | |
| 264 | NM_182706.4: | NP_874365.3: | 1208 | EREAGG | 840 | HLA-DRB1* | 20.24 | 26.19 | 1 | No | N/A | No |
| | c.2895del | p.Thr966Pro | | PLPPSP | | 13: | | | | | | |
| | | fsTer9 | | LPHSSP | | 01 | | | | | | |
| | | | | PTAAVA | | | | | | | | |
| | | | | TTSITT | | | | | | | | |
| | | | | ATPGVP | | | | | | | | |
| | | | | GLPS | | | | | | | | |
| 265 | NM_000937.4: | NP_000928.1: | 1235 | MHGGGP | 838 | HLA-DRB1* | 15.47 | 104.61 | 1 | No | N/A | No |
| | c.21del | p.Ser8Arg | | PSGDSA | | 15: | | | | | | |
| | | fsTer19 | | CPLRTI | | 01 | | | | | | |
| | | | | KRVQFG | | | | | | | | |
| | | | | VLSP | | | | | | | | |
| 266 | NM_001199198.2: | NP_001186127.1: | 1305 | AYIQSR | 987 | HLA-DRB1* | 38.21 | 13.27 | 2 | No | N/A | No |
| | c.1947del | p.Lys649Asn | | QALNSV | | 13: | | | | | | |
| | | fsTer18 | | VKITSK | | 02 | | | | | | |
| | | | | KKHPEL | | | | | | | | |
| | | | | ITFKYG | | | | | | | | |
| | | | | NSSASG | | | | | | | | |
| | | | | IEIL | | | | | | | | |
| 267 | NM_017514.3: | NP_059984.2: | 1335 | MPSVCL | 825 | HLA-DRB1* | 25.95 | 66.31 | 1 | No | N/A | No |
| | c.49del | p.Ala17Pro | | LLLLFL | | 04: | | | | | | |
| | | fsTer12 | | AVGGAL | | 05 | | | | | | |
| | | | | GNRPFR | | | | | | | | |
| | | | | AFVVTD | | | | | | | | |
| | | | | TTLTHL | | | | | | | | |
| | | | | A | | | | | | | | |
| 268 | NM_014866.1: | NP_055681.1: | 1348 | DGKFAN | 855 | HLA-DRB1* | 24.21 | 23.93 | 1 | No | N/A | No |
| | c.6197del | p.Pro2066Gln | | LTPSRT | | 13: | | | | | | |
| | | fsTer76 | | VPDSEA | | 01 | | | | | | |
| | | | | PPGWDR | | | | | | | | |
| | | | | ADSGPT | | | | | | | | |
| | | | | QPPLSL | | | | | | | | |
| | | | | SPAP | | | | | | | | |
| 269 | NM_005379.3: | NP_005370.1: | 1360 | VKVVQG | 988 | HLA-DRB1* | 39.78 | 72.67 | 3 | No | N/A | No |
| | c.3098del | p.Lys1033Arg | | PAGGDN | | 13: | | | | | | |
| | | fsTer8 | | SKLRYK | | 01 | | | | | | |
| | | | | KKGSHC | | | | | | | | |
| | | | | LEVTVQ | | | | | | | | |
| 270 | NM_001814.4: | NP_001805.3: | 1387 | IIYNQG | 799 | HLA-DRB1* | 77.79 | 77.33 | 1 | No | N/A | No |
| | c.315del | p.Phe105Leu | | FEIVLN | | 01: | | | | | | |
| | | fsTer10 | | DYKWFA | | 02 | | | | | | |
| | | | | FFKYKE | | | | | | | | |
| | | | | EGSKVT | | | | | | | | |
| | | | | TYCNET | | | | | | | | |
| | | | | MTGW | | | | | | | | |
| 271 | NM_001308120.1: | NP_001295049.1: | 1393 | SDEKRL | 847 | HLA-DRB1* | 13.78 | 15.62 | 2 | No | N/A | No |
| | c.502del | p.Glu168Arg | | CLQLLS | | 07: | | | | | | |
| | | fsTer11 | | DVLRGQ | | 01 | | | | | | |
| | | | | GEAGQL | | | | | | | | |
| | | | | EEAFSL | | | | | | | | |
| | | | | ALLPQL | | | | | | | | |
| | | | | VVSL | | | | | | | | |
| 272 | NM_002439.4: | NP_002430.3: | 1471 | STSYLL | 830 | HLA-DRB1* | 36.81 | 35.98 | 2 | No | N/A | yes |
| | c.1148del | p.Lys383Arg | | CISENK | | 13: | | | | | | |
| | | fsTer32 | | ENVRDK | | 02 | | | | | | |
| | | | | KKGNIF | | | | | | | | |
| | | | | IGIVGV | | | | | | | | |
| | | | | QPATGE | | | | | | | | |
| | | | | VVFD | | | | | | | | |
| 273 | NM_001009184.1: | NP_001009184.1: | 1521 | PYPQGG | 850 | HLA-DRB1* | 21.81 | 17.41 | 2 | No | N/A | No |
| | c.333del | p.Asn112Thr | | YPQGPY | | 07: | | | | | | |
| | | fsTer56 | | PQSPFP | | 01 | | | | | | |
| | | | | PNPYGQ | | | | | | | | |
| | | | | PQVFPG | | | | | | | | |
| | | | | QDPDSP | | | | | | | | |
| | | | | QHGN | | | | | | | | |
| 274 | NM_003358.1: | NP_003349.1: | 1539 | KLDYAV | 858 | HLA-DRB1* | 26.51 | 32.69 | 2 | No | N/A | No |
| | c.1094del | p.Leu365Cys | | AWFIRE | | 13: | | | | | | |
| | | fsTer9 | | SMTIYI | | 02 | | | | | | |
| | | | | FLSALW | | | | | | | | |
| | | | | DPTISW | | | | | | | | |
| | | | | RTGRYR | | | | | | | | |
| | | | | LRCG | | | | | | | | |
| 275 | NM_001037144.5: | NP_001032221.1: | 1572 | EKEERR | 833 | HLA-DRB1* | 36.72 | 39.22 | 1 | No | N/A | No |
| | c.1840del | p.Val612Tyr | | VWTMPP | | 15: | | | | | | |
| | | fsTer86 | | MAVALK | | 01 | | | | | | |
| | | | | PVLQQS | | | | | | | | |
| | | | | REARDE | | | | | | | | |
| | | | | LPGAPP | | | | | | | | |
| | | | | VLCS | | | | | | | | |
| 276 | NM_001136112.1: | NP_001129584.1: | 1706 | VPNTDQ | 871 | HLA-DQA1* | 104.11 | 167.91 | 2 | No | N/A | No |
| | c.241del | p.Thr81Gln | | KSTSVK | | 01: | | | | | | |
| | | fsTer26 | | KDNHKK | | 02/DQB1* | | | | | | |
| | | | | KTVKML | | 05: | | | | | | |
| | | | | EYLGKD | | 01 | | | | | | |
| | | | | VLHGVF | | | | | | | | |
| | | | | NYLA | | | | | | | | |
| 277 | NM_001114106.2: | NP_001107578.1: | 1842 | FNYNRA | 846 | HLA-DRB1* | 27.22 | 39.8 | 2 | No | N/A | No |
| | c.1722del | p.Phe574Leu | | FQVWAV | | 07: | | | | | | |
| | | fsTer4 | | PLLLVA | | 01 | | | | | | |
| | | | | FFAYLV | | | | | | | | |
| | | | | AHSFLS | | | | | | | | |
| | | | | VFETVL | | | | | | | | |
| | | | | DALF | | | | | | | | |
| 278 | NM_001164446.1: | NP_001157918.1: | 1988 | YATNPP | 823 | HLA-DQA1* | 92.38 | 18.59 | 1 | No | N/A | No |
| | c.140del | p.Gly47Ala | | WIFTQE | | 01: | | | | | | |
| | | fsTer15 | | APEEGT | | 02/DQB1* | | | | | | |
| | | | | GGFDGI | | 06: | | | | | | |
| | | | | YYGDNR | | 02 | | | | | | |
| | | | | FNTVSE | | | | | | | | |
| | | | | SGTA | | | | | | | | |
| 279 | NM_032387.4: | NP_115763.2: | 1995 | LSSSGF | 791 | HLA-DRB1* | 115.25 | 13.58 | 1 | No | N/A | No |
| | c.1822del | p.Val608Cys | | LDASDP | | 07: | | | | | | |
| | | fsTer53 | | ALQPPG | | 01 | | | | | | |
| | | | | GVPSSL | | | | | | | | |
| | | | | AESHLC | | | | | | | | |
| | | | | LPSAFA | | | | | | | | |
| | | | | LSIP | | | | | | | | |
| 280 | NM_012414.3: | NP_036546.2: | 2010 | LNIKKI | 866 | HLA-DRB1* | 47.05 | 23.15 | 1 | No | N/A | No |
| | c.2227del | p.Trp743Gly | | SEEEYV | | 15: | | | | | | |
| | | fsTer32 | | ALGSFF | | 01 | | | | | | |
| | | | | FWKCLH | | | | | | | | |
| | | | | GESSTE | | | | | | | | |
| | | | | DMCHTL | | | | | | | | |
| | | | | ESAG | | | | | | | | |
| 281 | NM_001042681.1: | NP_001036146.1: | 2064 | EKVASD | 811 | HLA-DRB1* | 100 | 15.48 | 2 | No | N/A | No |
| | c.2011del | p.Thr671Arg | | TEEADR | | 13: | | | | | | |
| | | fsTer159 | | TSSKKT | | 02 | | | | | | |
| | | | | KTQEIS | | | | | | | | |
| | | | | RPNSPS | | | | | | | | |
| | | | | EGEGES | | | | | | | | |
| | | | | SDSR | | | | | | | | |
| 282 | NM_007214.4: | NP_009145.1: | 2100 | KSKGPK | 806 | HLA-DRB1* | 93.14 | 34.43 | 1 | No | N/A | No |
| | c.1605del | p.Lys535Asn | | KTAKSK | | 07: | | | | | | |
| | | fsTer28 | | KKKPLK | | 01 | | | | | | |
| | | | | KKPTPV | | | | | | | | |
| | | | | LLPQSK | | | | | | | | |
| | | | | QQKQKQ | | | | | | | | |
| | | | | ANGV | | | | | | | | |
| 283 | NM_019062.1: | NP_061935.1: | 2196 | DNTWSI | 989 | HLA-DRB1* | 77.98 | 42.62 | 1 | No | N/A | No |
| | c.281del | p.Gly94Ala | | TCPLCR | | 15: | | | | | | |
| | | fsTer52 | | KVTAVP | | 01 | | | | | | |
| | | | | GGLICS | | | | | | | | |
| | | | | LRDHEA | | | | | | | | |
| | | | | VVGQLA | | | | | | | | |
| | | | | QPCT | | | | | | | | |
| 284 | NM_015306.2: | NP_056121.2: | 2230 | IIKCIE | 810 | HLA-DRB1* | 107.89 | 34.61 | 1 | No | N/A | No |
| | c.1841del | p.Asn614Thr | | DIKRPG | | 04: | | | | | | |
| | | fsTer34 | | EWSGLE | | 05 | | | | | | |
| | | | | KNKKDG | | | | | | | | |
| | | | | FKSSQL | | | | | | | | |
| | | | | NNPQFV | | | | | | | | |
| | | | | WVVP | | | | | | | | |
| 285 | NM_001184772.2: | NP_001171701.1: | 2301 | PISIID | 842 | HLA-DRB1* | 94.4 | 5.74 | 1 | No | N/A | No |
| | c.2306del | p.Lys769Arg | | QGEPKG | | 13: | | | | | | |
| | | fsTer14 | | TGATCG | | 01 | | | | | | |
| | | | | KKGSQA | | | | | | | | |
| | | | | GAEGQP | | | | | | | | |
| | | | | STVKRY | | | | | | | | |
| | | | | TPAR | | | | | | | | |
| 286 | NM_020762.2: | NP_065813.1: | 2341 | QTEMRV | 873 | HLA-DRB1* | 15.8 | 6.72 | 2 | No | N/A | No |
| | c.168del | p.Ala57Leu | | QLLQDL | | 13: | | | | | | |
| | | fsTer11 | | QDFFRK | | 02 | | | | | | |
| | | | | KAEIET | | | | | | | | |
| | | | | EYSRNL | | | | | | | | |
| | | | | EKLAER | | | | | | | | |
| | | | | FMAK | | | | | | | | |
| 287 | NM_018031.3: | NP_060501.3: | 2397 | GGPQDP | 857 | HLA-DRB1* | 28.51 | 30.6 | 2 | No | N/A | No |
| | c.2511del | p.Arg838Gly | | QPGLTA | | 13: | | | | | | |
| | | fsTer33 | | HVVSAG | | 02 | | | | | | |
| | | | | GRAEMH | | | | | | | | |
| | | | | CFSIMV | | | | | | | | |
| | | | | TPDPST | | | | | | | | |
| | | | | PSRL | | | | | | | | |
| 288 | NM_001130528.2: | NP_001124000.1: | 2432 | RWTEMI | 990 | HLA-DRB1* | 103.08 | 18.44 | 1 | No | N/A | No |
| | c.1645del | p.Arg549Gly | | RASREN | | 07: | | | | | | |
| | | fsTer28 | | PAMQEK | | 01 | | | | | | |
| | | | | KRSSIW | | | | | | | | |
| | | | | QFFSRL | | | | | | | | |
| | | | | FSSSSN | | | | | | | | |
| | | | | TTKK | | | | | | | | |
| 289 | NM_002458.2: | NP_002449.2: | 2529 | NPQRAQ | 835 | HLA-DRB1* | 58.68 | 2364.48 | 1 | No | N/A | No |
| | c.1100del | p.Pro367Gln | | LCEDHC | | 13: | | | | | | |
| | | fsTer105 | | VDGCFC | | 02 | | | | | | |
| | | | | PPGTVL | | | | | | | | |
| | | | | DDITHS | | | | | | | | |
| | | | | GCLPLG | | | | | | | | |
| | | | | QCPC | | | | | | | | |
| 290 | NM_001304717.2: | NP_001291646.2: | 2546 | CSIERA | 822 | HLA-DRB1* | 96.45 | 66.83 | 3 | Tested | No | No |
| | c.1487del | p.Asn496Met | | DNDKEY | | 13: | | | | | | |
| | | fsTer21 | | LVLTLT | | 02 | | | | | | |
| | | | | KNDLDK | | | | | | | | |
| | | | | ANKDKA | | | | | | | | |
| | | | | NRYFSP | | | | | | | | |
| | | | | NFKV | | | | | | | | |
| 291 | NM_005445.3: | NP_005436.1: | 2598 | SSKHNV | 837 | HLA-DRB1* | 49.26 | 54.42 | 1 | No | N/A | No |
| | c.127del | p.Tyr43Met | | IVGRNG | | 07: | | | | | | |
| | | fsTer69 | | SGKSNF | | 01 | | | | | | |
| | | | | FYAIQFVL | | | | | | | | |
| | | | | SDEFSH | | | | | | | | |
| | | | | LRPEQR | | | | | | | | |
| | | | | LA | | | | | | | | |
| 292 | NM_014935.4: | NP_055750.2: | 2668 | AQRKSS | 831 | HLA-DRB1* | 55.7 | 38.45 | 2 | No | N/A | No |
| | c.982del | p.Val328Tyr | | MNQLQQ | | 15: | | | | | | |
| | | fsTer172 | | WVNLRR | | 01 | | | | | | |
| | | | | GVPPPE | | | | | | | | |
| | | | | DLRSPS | | | | | | | | |
| | | | | RFYPVS | | | | | | | | |
| | | | | RRVP | | | | | | | | |
| 293 | NM_016044.2: | NP_057128.2: | 2709 | WVSQFV | 991 | HLA-DRB1* | 23.37 | 17.6 | 2 | No | N/A | yes |
| | c.842del | p.Pro281Gln | | TFYPGD | | 13: | | | | | | |
| | | fsTer26 | | VILTGT | | 01 | | | | | | |
| | | | | PPGVGV | | | | | | | | |
| | | | | FRKPPV | | | | | | | | |
| | | | | FLKKGD | | | | | | | | |
| | | | | EVQC | | | | | | | | |
| 294 | NM_017672.5: | NP_060142.3: | 2770 | EGGNLP | 844 | HLA-DRB1* | 59.96 | 45.7 | 1 | No | N/A | No |
| | c.1057del | p.Thr353His | | DAAEPD | | 15: | | | | | | |
| | | fsTer16 | | IISTIK | | 01 | | | | | | |
| | | | | KTFNFG | | | | | | | | |
| | | | | QNEALH | | | | | | | | |
| | | | | LFQTLM | | | | | | | | |
| | | | | ECMK | | | | | | | | |
| 295 | NM_002184.3: | NP_002175.2: | 2800 | KAYLKQ | 841 | HLA-DRB1* | 58.88 | 14.88 | 2 | No | N/A | No |
| | c.1587del | p.Val530Ter | | APPSKG | | 07: | | | | | | |
| | | | | PTVRTK | | 01 | | | | | | |
| | | | | KVGKNE | | | | | | | | |
| | | | | AVLEWD | | | | | | | | |
| | | | | QLPVDV | | | | | | | | |
| | | | | QNGF | | | | | | | | |
| 296 | NM_001170790.1: | NP_001164261.1: | 2817 | AFVGLL | 992 | HLA-DRB1* | 42.77 | 32.21 | 1 | No | N/A | No |
| | c.402del | p.Lys134Asn | | ASCLGL | | 07: | | | | | | |
| | | fsTer67 | | ELSRCR | | 01 | | | | | | |
| | | | | AKPPGR | | | | | | | | |
| | | | | ACSNPS | | | | | | | | |
| | | | | FLRFQL | | | | | | | | |
| | | | | DFYQ | | | | | | | | |
| 297 | NM_017552.3: | NP_060022.2: | 2831 | LVARAL | 993 | HLA-DRB1* | 13.65 | 6.9 | 1 | No | N/A | No |
| | c.1404del | p.Phe468Leu | | ANECSQ | | 13: | | | | | | |
| | | fsTer9 | | GDKKVA | | 02 | | | | | | |
| | | | | FFMRKG | | | | | | | | |
| | | | | ADCLSK | | | | | | | | |
| | | | | WVGESE | | | | | | | | |
| | | | | RQLR | | | | | | | | |
| 298 | NM_022167.2: | NP_071450.2: | 2949 | VNQEVL | 859 | HLA-DRB1* | 48.64 | 10.17 | 3 | No | N/A | No |
| | c.1584del | p.Gly529Ala | | EILDFH | | 15: | | | | | | |
| | | fsTer78 | | LYGSYP | | 01 | | | | | | |
| | | | | PGTPAL | | | | | | | | |
| | | | | KAYWEN | | | | | | | | |
| | | | | TYDAAD | | | | | | | | |
| | | | | GPSG | | | | | | | | |
| 299 | NM_001285.3: | NP_001276.2: | 2951 | GVYSRY | 826 | HLA-DRB1* | 81.38 | 1325.16 | 1 | No | N/A | No |
| | c.2022del | p.Val675Cys | | FTTYDT | | 01: | | | | | | |
| | | fsTer14 | | NGRYSV | | 02 | | | | | | |
| | | | | KVRALG | | | | | | | | |
| | | | | GVNAAR | | | | | | | | |
| | | | | RRVIPQ | | | | | | | | |
| | | | | QSGA | | | | | | | | |
| 300 | NM_003453.4: | NP_003444.1: | 3072 | LPPVFG | 862 | HLA-DRB1* | 99.31 | 32.92 | 1 | No | N/A | No |
| | c.3131del | p.Lys1044Arg | | EEYEEQ | | 15: | | | | | | |
| | | fsTer33 | | PRPRSK | | 01 | | | | | | |
| | | | | KKGAKR | | | | | | | | |
| | | | | KAVSGY | | | | | | | | |
| | | | | QSHDDS | | | | | | | | |
| | | | | SDNS | | | | | | | | |
| 301 | NM_004380.2: | NP_004371.2: | 3110 | GVDVCF | 994 | HLA-DRB1* | 30.42 | 96.53 | 2 | No | N/A | No |
| | c.4268del | p.Pro1423Leu | | FGMHVQ | | 07: | | | | | | |
| | | fsTer36 | | EYGSDC | | 01 | | | | | | |
| | | | | PPPNTR | | | | | | | | |
| | | | | RVYISY | | | | | | | | |
| | | | | LDSIHF | | | | | | | | |
| | | | | FRPR | | | | | | | | |
| 302 | NM_000038.5: | NP_000029.2: | 3295 | NQTTQE | 839 | HLA-DRB1* | 72.02 | 5.48 | 1 | No | V/A | No |
| | c.3927_3931del | p.Glu1309Asp | | ADSANT | | 03: | | | | | | |
| | | fsTer4 | | LQIAEI | | 01 | | | | | | |
| | | | | KEKIGT | | | | | | | | |
| | | | | RSAEDP | | | | | | | | |
| | | | | VSEVPA | | | | | | | | |
| | | | | VSQH | | | | | | | | |
| 303 | NM_019048.2: | NP_061921.1: | 3380 | TASALL | 995 | HLA-DRB1* | 103.08 | 20.92 | 2 | No | N/A | No |
| | c.1837del | p.Met613Trp | | PKRAMQ | | 13: | | | | | | |
| | | fsTer20 | | FGSRIA | | 02 | | | | | | |
| | | | | KMEKIN | | | | | | | | |
| | | | | EKASDK | | | | | | | | |
| | | | | CGRLQI | | | | | | | | |
| | | | | MSLE | | | | | | | | |
| 304 | NM_005895.3: | NP_005886.2: | 3419 | PRGPKV | 996 | HLA-DRB1* | 40.44 | 20.74 | 1 | No | N/A | No |
| | c.709del | p.Thr237Leu | | GSLGLP | | 13: | | | | | | |
| | | fsTer37 | | AHPREK | | 01 | | | | | | |
| | | | | KTSKSS | | | | | | | | |
| | | | | KIRSLA | | | | | | | | |
| | | | | DYRTED | | | | | | | | |
| | | | | SNAG | | | | | | | | |
| 305 | NM_001128430.1: | NP_001121902.1: | 3443 | LKQKFS | 809 | HLA-DRB1* | 122.2 | 27.16 | 1 | No | N/A | No |
| | c.1040del | p.Asn347Met | | MKAQNG | | 12: | | | | | | |
| | | fsTer24 | | FNKKRK | | 01 | | | | | | |
| | | | | KNVFNP | | | | | | | | |
| | | | | KRVVED | | | | | | | | |
| | | | | SEYDSG | | | | | | | | |
| | | | | SDVG | | | | | | | | |
| 306 | NM_022453.2: | NP_071898.2: | 3457 | SLRQQE | 836 | HLA-DRB1* | 108.92 | 11.95 | 2 | No | N/A | No |
| | c.749del | p.Gly250Glu | | ERKRLY | | 04: | | | | | | |
| | | fsTer29 | | QRQQER | | 03 | | | | | | |
| | | | | GGIIDL | | | | | | | | |
| | | | | EAERNR | | | | | | | | |
| | | | | YFISLQ | | | | | | | | |
| | | | | QPPA | | | | | | | | |
| 307 | NM_012434.4: | NP_036566.1: | 3524 | GKKYQW | 997 | HLA-DRB1* | 67.09 | 20.52 | 2 | No | N/A | No |
| | c.349del | p.Tyr117Met | | DAETQG | | 13: | | | | | | |
| | | fsTer17 | | WILGSF | | 02 | | | | | | |
| | | | | FYGYII | | | | | | | | |
| | | | | TQIPGG | | | | | | | | |
| | | | | YVASKI | | | | | | | | |
| | | | | GGKM | | | | | | | | |
| 308 | NM_017607.3: | NP_060077.1: | 3594 | ISLQDL | 998 | HLA-DRB1* | 21.02 | 23 | 1 | No | N/A | No |
| | c.1110del | p.Ile373Ser | | SKERRP | | 13: | | | | | | |
| | | fsTer51 | | GGAGGP | | 01 | | | | | | |
| | | | | PIQDED | | | | | | | | |
| | | | | EGEEGP | | | | | | | | |
| | | | | TEPPPA | | | | | | | | |
| | | | | EPRT | | | | | | | | |
| 309 | NM_202470.2: | NP_974199.1: | 3608 | EPGPLG | 795 | HLA-DQA1* | 154.28 | 43.65 | 1 | No | N/A | No |
| | c.149del | p.Pro50Leu | | GGGSGG | | 01: | | | | | | |
| | | fsTer48 | | PQMGLP | | 02/DQB1* | | | | | | |
| | | | | PPPPAL | | 06: | | | | | | |
| | | | | RPRLVF | | 02 | | | | | | |
| | | | | HTQLAH | | | | | | | | |
| | | | | GSPT | | | | | | | | |
| 310 | NM_001321829.1: | NP_001308758.1: | 3796 | LSLEIN | 920 | HLA-DRB1* | 129.13 | 32.63 | 3 | No | N/A | No |
| | c.2600del | p.Asn867Ile | | RKLQAV | | 03: | | | | | | |
| | | fsTer4 | | LEDTLL | | 01 | | | | | | |
| | | | | KNITLK | | | | | | | | |
| | | | | ENLQTL | | | | | | | | |
| | | | | GTEIER | | | | | | | | |
| | | | | LIKH | | | | | | | | |
| 311 | NM_001024847.2: | NP_001020018.1: | 3869 | HDFILE | 884 | HLA-DRB1* | 44.96 | 39.12 | 5 | Tested | No | yes |
| | c.458del | p.Lys153Ser | | DAASPK | | 07: | | | | | | |
| | | fsTer35 | | CIMKEK | | 01 | | | | | | |
| | | | | KKPGET | | | | | | | | |
| | | | | FFMCSC | | | | | | | | |
| | | | | SSDECN | | | | | | | | |
| | | | | DNII | | | | | | | | |
| 312 | NM_012231.4: | NP_036363.2: | 3873 | NKHAAF | 895 | HLA-DRB1* | 43.36 | 19.82 | 4 | No | N/A | No |
| | c.4467del | p.Val1490Phe | | SCPKKP | | 10: | | | | | | |
| | | fsTer74 | | LSPPKK | | 01 | | | | | | |
| | | | | KVSHSS | | | | | | | | |
| | | | | KKGGHS | | | | | | | | |
| | | | | SPASSD | | | | | | | | |
| | | | | KNSN | | | | | | | | |
| 313 | NM_000722.3: | NP_000713.2: | 3897 | MAAGCL | 999 | HLA-DRB1* | 35.76 | 21.48 | 2 | No | N/A | No |
| | c.41del | p.Phe14Ser | | LALTLT | | 13: | | | | | | |
| | | fsTer66 | | LFQSLL | | 02 | | | | | | |
| | | | | IGPSSE | | | | | | | | |
| | | | | EPFPSA | | | | | | | | |
| | | | | VTIK | | | | | | | | |
| 314 | NM_001271634.1: | NP_001258563.1: | 3907 | VASHPE | 1000 | HLA-DRB1* | 55.08 | 28.03 | 2 | No | N/A | No |
| | c.356del | p.Leu119Cys | | TRSAFL | | 13: | | | | | | |
| | | fsTer26 | | AAHIPL | | 02 | | | | | | |
| | | | | FLYPFL | | | | | | | | |
| | | | | HTVSKT | | | | | | | | |
| | | | | RPFEYL | | | | | | | | |
| | | | | RLTS | | | | | | | | |
| 315 | NM_001297568.1: | NP_001284497.1: | 4246 | CQKCGK | 1001 | HLA-DRB1* | 48.33 | 18.59 | 2 | No | N/A | No |
| | c.1015del | p.Thr339Leu | | AFSRAS | | 10: | | | | | | |
| | | fsTer31 | | TLWKHK | | 01 | | | | | | |
| | | | | KTHTGE | | | | | | | | |
| | | | | KPYKCK | | | | | | | | |
| | | | | KM | | | | | | | | |
| 316 | NM_182641.3: | NP_872579.2: | 4264 | QVMKYI | 853 | HLA-DRB1* | 74.59 | 39.3 | 2 | No | N/A | No |
| | c.7776del | p.Lys2592Asn | | LDKIDK | | 13: | | | | | | |
| | | fsTer36 | | EEKQAA | | 02 | | | | | | |
| | | | | KKRKRE | | | | | | | | |
| | | | | ESVEQK | | | | | | | | |
| | | | | RSKQNA | | | | | | | | |
| | | | | TKLS | | | | | | | | |
| 317 | NM_024079.4: | NP_076984.2: | 4480 | DVLFVY | 874 | HLA-DRB1* | 62.44 | 34.91 | 1 | No | N/A | No |
| | c.396del | p.Val133Trp | | AVRECC | | 01: | | | | | | |
| | | fsTer24 | | KCIDGK | | 02 | | | | | | |
| | | | | KVGKEL | | | | | | | | |
| | | | | TEKPKF | | | | | | | | |
| | | | | ILSVLL | | | | | | | | |
| | | | | LWNF | | | | | | | | |

SUPPLEMENTARY TABLE 7
List of the Top 100 most recurrent predicted MHC-II neoAgs,
with higher immunogenicity, obtained from the
computational methods in the discovery set.

| | | | | | | Micro- | Reference | | Altered | Number | |
|---|---|---|---|---|---|---|---|---|---|---|---|
| Mutant | SEQ | | | | | satellite | MS | Var- | MS | deleted | Pep- |
| Epitope | ID | Gene | Chromo- | | | motif | Lengths | iant | Length | nucleo- | tide |
| Sequence | NO | Name | some | Start | Stop | | (repeats) | Type | (repeats) | tides | Length |
| QLARFFPITPPVWHI | 234 | RNF43 | chr17 | 56435160 | 56435161 | G | 7 | FS | 6 | −1 | 15 |
| LRGGVIQSTRRRRRA | 318 | ELMSAN1 | chr14 | 74205772 | 74205773 | C | 7 | FS | 6 | −1 | 15 |
| PCASLLSTLSQPPPQ | 319 | DOCK3 | chr3 | 51417603 | 51417604 | C | 7 | FS | 6 | −1 | 15 |
| LEVMLLNMGYRITGL | 239 | WDTC1 | chr1 | 27621107 | 27621108 | G | 8 | FS | 7 | −1 | 15 |
| EDMQEVVVHKKRGLF | 320 | ACVR2A | chr2 | 148683685 | 148683686 | A | 8 | FS | 7 | −1 | 15 |
| RFPLLMMWRTPMTTR | 321 | MICAL3 | chr22 | 18300931 | 18300932 | C | 7 | FS | 6 | −1 | 15 |
| PVLFKADQSESSLSS | 322 | CHD3 | chr17 | 7798764 | 7798765 | C | 7 | FS | 6 | −1 | 15 |
| RIPAVLRTEGEPLHT | 323 | ASTE1 | chr3 | 130733046 | 130733047 | A | 11 | FS | 10 | −1 | 15 |
| FGILNVLEFSSDRKK | 324 | ATP8A2 | chr13 | 26151212 | 26151212 | A | 10 | FS | 11 | 4 | 15 |
| ILVALSWMGGLHSFY | 325 | OR4M1 | chr14 | 20248930 | 20248930 | G | 6 | FS | 7 | 1 | 15 |
| LGQIMASAVEASQPP | 326 | MFRP | chr11 | 119213687 | 119213688 | G | 7 | FS | 6 | −1 | 15 |
| PALLLAEATHKASAL | 229 | TCF7L2 | chr10 | 114925316 | 114925317 | A | 9 | FS | 8 | −1 | 15 |
| TVEMRRWWTLVMEWK | 250 | TCF20 | chr22 | 42564715 | 42564716 | C | 7 | FS | 6 | −1 | 15 |
| KSLVRLSSCVPVALM | 311 | TGFBR2 | chr3 | 30691871 | 30691872 | A | 10 | FS | 9 | −1 | 15 |
| QRWLTSTTSRSSALM | 327 | LARP4B | chr10 | 890938 | 890939 | A | 7 | FS | 6 | −1 | 15 |
| LLHWRIGGGTPLSIS | 328 | ARID1A | chr1 | 27105930 | 27105931 | G | 7 | FS | 6 | −1 | 15 |
| EIQLTMNDSKHKLES | 329 | BMPR2 | chr2 | 203420129 | 203420130 | A | 7 | FS | 6 | −1 | 15 |
| LTCALHNDGIYIMSR | 330 | KLHL42 | chr12 | 27950768 | 27950769 | G | 7 | FS | 6 | −1 | 15 |
| CILVALSWRGASFIL | 331 | OR4M2 | chr15 | 22369023 | 22369024 | G | 7 | FS | 6 | −1 | 15 |
| RHVIKVLLGRKVNWH | 233 | UBR5 | chr8 | 103289348 | 103289349 | T | 8 | FS | 7 | −1 | 15 |
| ACRLRWARPEPAAQA | 312 | PRDM2 | chr1 | 14108748 | 14108749 | A | 9 | FS | 8 | −1 | 15 |
| GVRILKLCSKVSFRV | 332 | CASP5 | chr11 | 104879686 | 104879687 | A | 10 | FS | 9 | −1 | 15 |
| LRAVVVDDYRRRKKR | 333 | WDR55 | chr5 | 140049101 | 140049102 | A | 8 | FS | 7 | −1 | 15 |
| ALGLRILPPPLTSPS | 334 | CELSR1 | chr22 | 46931226 | 46931227 | G | 6 | FS | 5 | −1 | 15 |
| KKSVLKAIEQADLLQ | 335 | AP1S1 | chr7 | 100802404 | 100802405 | G | 8 | FS | 7 | −1 | 15 |
| KEALFLQEVFQAERL | 336 | MARCKS | chr6 | 114181209 | 114181211 | A | 11 | FS | 9 | −1 | 15 |
| WTPRKLVGRAVRRKG | 337 | USP35 | chr11 | 77920855 | 77920856 | C | 8 | FS | 7 | −1 | 15 |
| ADRAFMAAQKCHKKT | 338 | ESRP1 | chr8 | 95686610 | 95686611 | A | 8 | FS | 7 | −1 | 15 |
| FGYATSISMAQASDG | 339 | TMEM94 | chr17 | 73491062 | 73491063 | C | 7 | FS | 6 | −1 | 15 |
| KYRCFSYLPISPTFV | 340 | SLC23A2 | chr20 | 4850568 | 4850569 | C | 9 | FS | 8 | −1 | 15 |
| LRGELSYNLGAGEAL | 341 | KCNH3 | chr12 | 49948319 | 49948320 | G | 5 | FS | 4 | −1 | 15 |
| ARHRLASFKTVIKKR | 342 | CCDC15 | chr11 | 124845048 | 124845049 | A | 8 | FS | 7 | −1 | 15 |
| LRELRLDNSVAIHYI | 343 | TMEM132D | chr12 | 130184704 | 130184705 | C | 7 | FS | 6 | −1 | 15 |
| AAPEIILGNPVSLTS | 344 | TRIO | chr5 | 14487780 | 14487781 | C | 7 | FS | 6 | −1 | 15 |
| RLSLVQSSSWPTVLH | 345 | SLC22A9 | chr11 | 63149670 | 63149671 | A | 11 | FS | 10 | −1 | 15 |
| SRQPSPLLLLPPLPA | 346 | CIC | chr19 | 42778293 | 42778294 | C | 5 | FS | 4 | −1 | 15 |
| KRLMSLSPGRPPLLL | 347 | BAX | chr19 | 49458970 | 49458971 | G | 8 | FS | 7 | −1 | 15 |
| KSLLFPSAPASVMNA | 348 | LIPE | chr19 | 42905972 | 42905973 | C | 6 | FS | 5 | −1 | 15 |
| QGKLQQHHVLRVSRR | 349 | DAPK1 | chr9 | 90321801 | 90321801 | G | 7 | FS | 8 | 1 | 15 |
| AEIRAQDAPLSLLQT | 350 | NES | chr1 | 156642803 | 156642804 | C | 7 | FS | 6 | −1 | 15 |
| HGSHNIKKAWYLIAM | 351 | TMEM60 | chr7 | 77423459 | 77423460 | A | 9 | FS | 8 | −1 | 15 |
| RCCLRTSCGAARPRR | 352 | MYCN | chr2 | 16082313 | 16082314 | C | 7 | FS | 6 | −1 | 15 |
| QKKLMLLRLNLRKMC | 223 | SEC31A | chr4 | 83785564 | 83785565 | A | 9 | FS | 8 | −1 | 15 |
| SINVLCVRASLIEKL | 240 | SPINK5 | chr5 | 147499874 | 147499875 | A | 10 | FS | 9 | −1 | 15 |
| NSKLRYKKRGVIAWR | 269 | MYO1A | chr12 | 57422572 | 57422573 | A | 8 | FS | 7 | −1 | 15 |
| ANRYFSPNFKVKLYF | 290 | PTEN | chr10 | 89720811 | 89720812 | A | 6 | FS | 5 | −1 | 15 |
| KERLFRNFGGLLGPL | 298 | XYLT2 | chr17 | 48433966 | 48433967 | C | 7 | FS | 6 | −1 | 15 |
| KLQAVLEDTLLKILL | 310 | CCDC186 | chr10 | 115885657 | 115885658 | A | 6 | FS | 5 | −1 | 15 |
| LCNRLLKSFSKWSLV | 353 | AASDH | chr4 | 57220268 | 57220269 | T | 10 | FS | 9 | −1 | 15 |
| PVTPLRVQSVLLLGV | 354 | SPECC1 | chr17 | 20108262 | 20108263 | A | 8 | FS | 7 | −1 | 15 |
| IAGYRESAAFLLRSA | 355 | NOL4L | chr20 | 31041555 | 31041556 | C | 8 | FS | 7 | −1 | 15 |
| SGTARLARTAIAAST | 356 | ZFP36L2 | chr2 | 43452622 | 43452623 | G | 6 | FS | 5 | −1 | 15 |
| NQPVEFNHAINYLIR | 357 | SIN3A | chr15 | 75703909 | 75703910 | NA | NA | FS | NA | −1 | 15 |
| RRPLRSWTPRTRGAH | 358 | CNKSR1 | chr1 | 26510310 | 26510311 | C | 7 | FS | 6 | −1 | 15 |
| NEIQKLQKTLKKKPR | 359 | GBP3 | chr1 | 89473441 | 89473442 | A | 10 | FS | 9 | −1 | 15 |
| PRLVKMISSISLEIW | 360 | CRIM1 | chr2 | 36764627 | 36764628 | C | 6 | FS | 5 | −1 | 15 |
| SSIFIGGSFILKKKA | 361 | NIPA2 | chr15 | 23021235 | 23021236 | G | 3 | FS | 2 | −1 | 15 |
| PALLLPATTCKVPRL | 362 | CLDN4 | chr7 | 73246062 | 73246063 | G | 6 | FS | 5 | −1 | 15 |
| KKRKKFMKDAKKRGR | 363 | DDX27 | chr20 | 47858503 | 47858504 | A | 8 | FS | 7 | −1 | 15 |
| REYMLNLFKALKRIH | 364 | CDC7 | chr1 | 91967356 | 91967357 | A | 9 | FS | 8 | −1 | 15 |
| APQGFRATLVPPALE | 365 | COL9A2 | chr1 | 40769746 | 40769747 | C | 6 | FS | 5 | −1 | 15 |
| TTVGLLRMAATRTSL | 366 | R3HDM2 | chr12 | 57648749 | 57648750 | G | 13 | FS | 12 | −1 | 15 |
| DTWAALEGLSSPFRE | 367 | RNF213 | chr17 | 78272285 | 78272288 | CT | 4 | in | 3 | −2 | 15 |
| | | | | | | | | frame | | | |
| | | | | | | | | _del | | | |
| KQGWLHKKGGGLLHA | 368 | MYO10 | chr5 | 16694605 | 16694605 | G | 8 | FS | 9 | 1 | 15 |
| LTETVYSTTQQIHSS | 369 | HOXA11 | chr7 | 27222461 | 27222462 | A | 9 | FS | 8 | −1 | 15 |
| NPAFDVTPTTSSLVA | 370 | MRI1 | chr19 | 13882967 | 13882968 | C | 6 | FS | 5 | −1 | 15 |
| ETNMGIIAGVAFGIA | 371 | TSPAN7 | chrX | 38535026 | 38535027 | C | 7 | FS | 6 | −1 | 15 |
| SSFFCRCRREYRVTM | 372 | COBLL1 | chr2 | 165551295 | 165551296 | T | 9 | FS | 8 | −1 | 15 |
| PGCFWPCLWNSLLRF | 373 | PLOD3 | chr7 | 100855926 | 100855927 | C | 7 | FS | 6 | −1 | 15 |
| FPILFFFSSKGVRAT | 374 | OR7E24 | chr19 | 9361740 | 9361741 | T | 11 | FS | 10 | −1 | 15 |
| IAYYIEGIENSVFFF | 375 | VPS13A | chr9 | 79954447 | 79954447 | T | 5 | FS | 13 | 1 | 15 |
| YDCYVAIRPLPYATR | 376 | OR1K1 | chr9 | 125562787 | 125562788 | C | 10 | FS | 9 | −1 | 15 |
| DRINANTITSPALLL | 377 | ADAMTS17 | chr15 | 100516255 | 100516256 | G | 6 | FS | 5 | −1 | 15 |
| HAFIVPIRSLQDHTH | 378 | ACOX2 | chr3 | 58517427 | 58517428 | C | 8 | FS | 7 | −1 | 15 |
| PKKKRSAFPSRSLSS | 379 | MARCKS | chr6 | 114181209 | 114181210 | A | 11 | FS | 10 | −1 | 15 |
| ILLGPLLPNVVFYIL | 380 | ATP2B1 | chr12 | 90005129 | 90005129 | A | 5 | FS | 10 | 4 | 15 |
| PPGVVLNNISSYASV | 381 | ARL10 | chr5 | 175796243 | 175796243 | T | 16 | FS | 15 | 1 | 15 |
| WEAAMMNGKVPFFSA | 382 | C22orf24 | chr22 | 32334104 | 32334105 | A | 9 | FS | 8 | −1 | 15 |
| SLQSAVSNIAQAPLY | 383 | CLCA2 | chr1 | 86921034 | 86921036 | NA | NA | FS | NA | −1 | 15 |
| RPQVRLAGAQAIFEA | 384 | TBC1D10C | chr11 | 67176564 | 67176565 | C | 7 | FS | 6 | −1 | 15 |
| PRESQIHSILSKMVQ | 385 | EPHA10 | chr1 | 38185237 | 38185238 | C | 6 | FS | 5 | −1 | 15 |
| KETLRLNPPVPGGFR | 386 | CYP26A1 | chr10 | 94835039 | 94835044 | GA | 4 | FS | 3 | -4 | 15 |
| SRRSLMSVASAYSAK | 387 | MAPK8IP1 | chr11 | 45907401 | 45907402 | G | 7 | FS | 6 | −1 | 15 |
| REAGRFAVLGTMVMK | 388 | ADAMTSL4 | chr1 | 150530505 | 150530506 | G | 8 | FS | 7 | −1 | 15 |
| HSTPFQAGTPVMMFL | 389 | NEUROD4 | chr12 | 55421127 | 55421128 | C | 6 | FS | 5 | −1 | 15 |
| RLMPKFLNSTNSWWT | 390 | MAPRE3 | chr2 | 27248516 | 27248517 | C | 8 | FS | 7 | −1 | 15 |
| LATAAAAAAAAAFGD | 391 | KDM6A | chrX | 44732825 | 44732825 | CCG | 7 | in | 9 | 5 | 15 |
| | | | | | | | | frame | | | |
| | | | | | | | | _ins | | | |
| KRLSKVETLRAAIDY | 392 | ASCL4 | chr12 | 108169096 | 108169097 | C | 5 | FS | 4 | −1 | 15 |
| LAQEQKKKKSWRLLL | 393 | CCDC150 | chr2 | 197531518 | 197531519 | A | 11 | FS | 10 | −1 | 15 |
| ITRQQWKKALRSMPK | 394 | SLAMF1 | chr1 | 160589600 | 160589601 | A | 9 | FS | 8 | −1 | 15 |
| SRPLAHSVASTLAPA | 395 | ESPNL | chr2 | 239039297 | 239039298 | G | 6 | FS | 5 | −1 | 15 |
| IGSISRQSSLSEKKF | 396 | KIF21A | chr12 | 39713783 | 39713784 | A | 9 | FS | 8 | −1 | 15 |
| GLCILKEAPRRKRPA | 397 | ZNF541 | chr19 | 48049093 | 48049094 | C | 7 | FS | 6 | −1 | 15 |
| RKMILSQLHHSMTRF | 398 | DGKD | chr2 | 234365951 | 234365952 | G | 7 | FS | 6 | −1 | 15 |
| ILQHFLLHATPQTQL | 399 | NOD2 | chr16 | 50745398 | 50745399 | C | 7 | FS | 6 | −1 | 15 |
| QAHLPSAPALPPPTH | 400 | IQSEC1 | chr3 | 12942840 | 12942841 | G | 7 | FS | 6 | −1 | 15 |
| GHSLVQMEPLTTARP | 401 | ELAVL3 | chr19 | 11577604 | 11577605 | G | 9 | FS | 8 | −1 | 15 |
| GVHSTIKVIKAKKKH | 402 | AIM2 | chr1 | 159032486 | 159032487 | A | 10 | FS | 9 | −1 | 15 |
| QGGLLMGYSPAGGRH | 403 | FAM214B | chr9 | 35108147 | 35108148 | G | 7 | FS | 6 | −1 | 15 |
| LLRMTLFSLVPLEPI | 404 | ACOX3 | chr4 | 8418187 | 8418188 | C | 4 | FS | 3 | −1 | 15 |

| | | | | | | | | Tumor | | | Eli- |
|---|---|---|---|---|---|---|---|---|---|---|---|
| SEQ | | | Immuno- | | SEQ | | Binding | Abun- | Sample | Eli- | spot |
| ID | | | genicity | Wildtype | ID | HLA | Affinity | dance | Recur- | sopt | reac- |
| NO | HGVSc | HGVSp | Score | sequence | NO | Allele | (nM) | (TPM) | rence | tested | tive |
| 234 | NM_017763.4:c. | NP_060233.3:p. | 381 | FNLQKSSLSARHPQRKR | 784 | HLA- | 109.73 | 79.41 | 8 | Tested | No |
| | 1976del | Gly659ValfsTer | | RGGPSEPTPGSRPQDATV | | DRB1*04:05 | | | | | |
| | | 41 | | HPACQ | | | | | | | |
| 318 | NM_001043318. | NP_001036783. | 36602 | PLGQSHLAHHSMAPYPF | 878 | HLA- | 16.03 | 9.23 | 7 | Tested | No |
| | 1:c.939del | 1:p.Asn314Thrf | | PPNPDMNPELRKALLQD | | DRB1*13:01 | | | | | |
| | | sTer4 | | SAPQPA | | | | | | | |
| 319 | NM_004947.4:c. | NP_004938.1:p. | 78669 | KGHYSLHFDAFHHPLGD | 877 | HLA- | 704.38 | 8.49 | 7 | Tested | No |
| | 5555del | Pro1852GlnfsTe | | TPPALPARTLRKSPLHPIP | | DRB1*04:03 | | | | | |
| | | r45 | | ASPT | | | | | | | |
| 239 | NM_001276252. | NP_001263181. | 476 | ATYVTFSPNGTELLVNM | 804 | HLA- | 30.05 | 10.01 | 6 | Tested | No |
| | 1:c.868del | 1:p.Glu290Asnf | | GGEQVYLFDLTYKQRPY | | DRB1*15:01 | | | | | |
| | | sTer8 | | TFLLPR | | | | | | | |
| 320 | NM_001278579. | NP_001265508. | 4522 | EIGQHPSLEDMQEVVVH | 889 | HLA- | 56.05 | 8.08 | 6 | Tested | No |
| | 1:c.1310del | 1:p.Lys437Argf | | KKKRPVLRDYWQKHAG | | DRB1*13:01 | | | | | |
| | | sTer5 | | MAMLCET | | | | | | | |
| 321 | NM_015241.2:c. | NP_056056.2:p. | 10447 | EPNASVVPPPLPATWMR | 880 | HLA- | 27.32 | 36.88 | 6 | Tested | No |
| | 4495del | Arg1499GlyfsT | | PPREPAQPPREEVRKSFV | | DRB1*13:01 | | | | | |
| | | er106 | | ESVEE | | | | | | | |
| 322 | NM_001005271. | NP_001005271. | 15344 | PVAVPAPQQADGNPDVP | 885 | HLA- | 308.49 | 98.14 | 6 | Tested | No |
| | 2:c.1795del | 2:p.Arg599Valfs | | PPRPLQGRSEREFFVKW | | DRB1*03:01 | | | | | |
| | | Ter16 | | VGLSYW | | | | | | | |
| 323 | NM_001288950. | NP_001275879. | 51952 | SYAPAEIFLPKGRSNSKK | 881 | HLA- | 359.76 | 4.09 | 6 | Tested | Yes |
| | 1:c.1969del | 1 :p.Arg657Glyf | | KRQKKQNTSCSKNRGRT | | DRB1*07:01 | | | | | |
| | | sTer33 | | TAHTK | | | | | | | |
| 324 | NM_016529.4:c. | NP_057613.4:p. | 133802 | GQEQTFGILNVLEFSSDR | 908 | HLA- | 453.63 | 1.00E−04 | 6 | Tested | No |
| | 1719_1723dup | Arg575LysfsTer | | KRMSVIVRTPSGRLRLY | | DRB1*01:01 | | | | | |
| | | 6 | | CKGAD | | | | | | | |
| 325 | NM_001005500. | NP_001005500. | 143257 | TIMNRRLCCIL VALSWM | 883 | HLA- | 33.76 | 0.07 | 6 | Tested | No |
| | 1:c.455dup | 1:p.Phe 153Leuf | | GGFIHSIIQVALIVRLPFC | | DRB1*01:01 | | | | | |
| | | sTer21 | | GPNE | | | | | | | |
| 326 | NM_031433.3:c. | NP_113621.1:p. | 147253 | SSSGAFSLLGRFCGAEPP | 882 | HLA- | 321.49 | 1.00E−04 | 6 | No | N/A |
| | 1150del | His384ThrfsTer | | PHLVSSHHELAVLFRTD | | DQA1*01:02 | | | | | |
| | | 94 | | HGISS | | /DQB1*03: | | | | | |
| | | | | | | 02 | | | | | |
| 229 | NM_001146274. | NP_001139746. | 263 | ALFGLDRQTLWCKPCRR | 788 | HLA- | 59.39 | 44.54 | 5 | Tested | No |
| | 1:c.1403del | 1 :p.Lys468Serfs | | KKKCVRYIQGEGSCLSPP | | DRB1*13:01 | | | | | |
| | | Ter23 | | SSDGS | | | | | | | |
| 250 | NM_005650.2:c. | NP_005641.1:p. | 785 | NFSVRCPKHKPPLPCPLP | 787 | HLA- | 101.72 | 39.27 | 5 | Tested | No |
| | 5826del | Leu1943CysfsT | | PLQNKTAKGSLSTEQSE | | DRB1*15:01 | | | | | |
| | | er118 | | RG | | | | | | | |
| 311 | NM_001024847. | NP_001020018. | 3869 | HDFILEDAASPKCIMKEK | 884 | HLA- | 44.96 | 39.12 | 5 | Tested | No |
| | 2:c.458del | 1 :p.Lys153Serfs | | KKPGETFFMCSCSSDEC | | DRB1*07:01 | | | | | |
| | | Ter35 | | NDNII | | | | | | | |
| 327 | NM_015155.2:c. | NP_055970.1:p. | 11885 | GGNESQPDSQEDPREVL | 887 | HLA- | 67.15 | 20.21 | 5 | No | N/A |
| | 487del | Thr163HisfsTer | | KKTLEFCLSRENLASDM | | DRB1*07:01 | | | | | |
| | | 47 | | YLISQM | | | | | | | |
| 328 | NM_006015.4:c. | NP_006006.3:p. | 19520 | LGRVQEFDSGLLHWRIG | 886 | HLA- | 18.34 | 28.68 | 5 | Tested | No |
| | 5548del | Asp1850ThrfsT | | GGDTTEHIQTHFESKTEL | | DRB1*01:01 | | | | | |
| | | er33 | | LPSRP | | | | | | | |
| 329 | NM_001204.6:c. | NP_001195.2:p. | 31532 | KNISSEHSMSSTPLTIGEK | 879 | HLA- | 218.58 | 5.18 | 5 | Tested | No |
| | 1748del | Asn583ThrfsTer | | NRNSINYERQQAQARIPS | | DRB1*13:01 | | | | | |
| | | 44 | | PET | | | | | | | |
| 330 | NM_020782.1:c. | NP_065833.1:p. | 126312 | HIRKQQMVSVEETIYIVG | 891 | HLA- | 61.42 | 1.00E−04 | 5 | No | N/A |
| | 1194del | Cys399ValfsTer | | GCLHELGPNRRSSQSED | | DRB1*13:02 | | | | | |
| | | 44 | | MLTVQ | | | | | | | |
| 331 | NM_001004719. | NP_001004719. | 135675 | ATIMNQRLCCILVALSW | 890 | HLA- | 303.55 | 1.00E−04 | 5 | No | N/A |
| | 2:c.455del | 2:p.Gly152Alafs | | RGGFIHSIIQVALIVRLPF | | DRB1*13:02 | | | | | |
| | | Ter23 | | CGPN | | | | | | | |
| 233 | NM_015902.5:c. | NP_056986.2:p. | 375 | MSYAANLKNVMNMQN | 797 | HLA- | 36.66 | 61.73 | 4 | Tested | No |
| | 6360del | Glu2121LysfsT | | RQKKEGEEQPVLPEETES | | DRB1*15:01 | | | | | |
| | | er28 | | SKPGPSA | | | | | | | |
| 312 | NM_012231.4:c. | NP_036363.2:p. | 3873 | NKHAAFSCPKKPLSPPK | 895 | HLA- | 43.36 | 19.82 | 4 | No | N/A |
| | 4467del | Val1490PhefsTe | | KKVSHSSKKGGHSSPAS | | DRB1*10:01 | | | | | |
| | | r74 | | SDKNSN | | | | | | | |
| 332 | NM_001136112. | NP_001129584. | 5659 | VPRVEGVFIFLIEDSGKK | 894 | HLA- | 61.82 | 10.67 | 4 | Tested | No |
| | 1:c.67del | 1:p. Arg23Glyfs | | KRRKNFEAMFKGILQSG | | DRB1*01:02 | | | | | |
| | | Ter21 | | LDNFV | | | | | | | |
| 333 | NM_017706.4:c. | NP_060176.2:p. | 11543 | WDMAQLRAVVVDDYR | 1002 | HLA- | 45.68 | 12.18 | 4 | No | N/A |
| | 1022del | Lys341 ArgfsTer | | RRKKKGGPLRALSSKTW | | DRB1*03:01 | | | | | |
| | | 8 | | STDDFFAG | | | | | | | |
| 334 | NM_014246.1:c. | NP_055061.1:p. | 11687 | GENARLHYRLVDTASTF | 896 | HLA- | 95.75 | 8.83 | 4 | Tested | No |
| | 1841del | Gly614AlafsTer | | LGGGSAGPKNPAPTPDF | | DRB1*15:01 | | | | | |
| | | 54 | | PFQIHN | | | | | | | |
| 335 | NM_001283.3:c. | NP_001274.1:p. | 11891 | IIFNFEKAYFILDEFLMG | 818 | HLA- | 129.04 | 19.86 | 4 | Tested | No |
| | 364del | Asp122MetfsTe | | GDVQDTSKKSVLKAIEQ | | DRB1*10:01 | | | | | |
| | | r11 | | ADLLQ | | | | | | | |
| 336 | NM_002356.5:c. | NP_002347.5:p. | 16707 | PKAEDGATPSPSNETPKK | 888 | HLA- | 167.6 | 6 | 4 | Tested | No |
| | 463_464del | Lys155GlufsTer | | KKKRFSFKKSFKLSGFSF | | DRB1*04:05 | | | | | |
| | | 28 | | KKNK | | | | | | | |
| 337 | NM_020798.2:c. | NP_065849.1:p. | 18382 | GRVGPRRQRKHCITEDT | 924 | HLA- | 25.2 | 2.32 | 4 | No | N/A |
| | 1962del | Thr655ProfsTer | | PPTSLYIEGLDSKEAGGQ | | DRB1*13:01 | | | | | |
| | | 23 | | SSQEE | | | | | | | |
| 338 | NM_017697.3:c. | NP_060167.2:p. | 18823 | IQMKSADRAFMAAQKC | 1003 | HLA- | 73.64 | 61.98 | 4 | No | N/A |
| | 1535del | Asn512ThrfsTer | | HKKNMKDRYVEVFQCS | | DRB1*11:04 | | | | | |
| | | 2 | | AEEMNFVL | | | | | | | |
| 339 | NM_001321148. | NP_001308077. | 31657 | NCHISLTPNGDMPGSEIP | 899 | HLA- | 42.34 | 57.53 | 4 | No | N/A |
| | 1:c.2712del | 1 :p.Ser905Profs | | PSSPSHAGSLHDDLNQV | | DQA1*01:02 | | | | | |
| | | Ter13 | | SRDDA | | /DQB1*06:0 | | | | | |
| | | | | | | 2 | | | | | |
| 340 | NM_005116.5:c. | NP_005107.4:p. | 36405 | ESIGDYYACARLSCAPPP | 900 | HLA- | 28.92 | 3.56 | 4 | No | N/A |
| | 1233del | Ile412SerfsTer4 | | PIHAINRGIFVEGLSCVL | | DRB1*07:01 | | | | | |
| | | | | DGIF | | | | | | | |
| 341 | NM_012284.1:c. | NP_036416.1:p. | 105649 | APRFSRGLRGELSYNLG | 947 | HLA- | 156.6 | 1.00E−04 | 4 | No | N/A |
| | 2123del | Gly708GlufsTer | | AGGGSAEVDTSSLSGDN | | DRB1*13:02 | | | | | |
| | | 11 | | TLMSTL | | | | | | | |
| 342 | NM_025004.2:c. | NP_079280.2:p. | 113666 | ETMKQARHRLASFKTVI | 909 | HLA- | 273.06 | 1.00E−04 | 4 | No | N/A |
| | 581del | Lys 194ArgfsTer | | KKKGSVFPDDGRKSFLT | | DRB1*04:03 | | | | | |
| | | 29 | | REEVLS | | | | | | | |
| 343 | NM_133448.2:c. | NP_597705.2:p. | 115119 | DLGLCVAELELLSSWFSP | 901 | HLA- | 10.26 | 1.00E−04 | 4 | No | N/A |
| | 618del | Thr207ArgfsTer | | PTVVAGRRKSVDQPEGT | | DRB1*13:02 | | | | | |
| | | 75 | | PVELY | | | | | | | |
| 344 | NM_007118.2:c. | NP_009049.2:p. | 117398 | STSRSRPSRIPQPVRHHPP | 905 | HLA- | 20.85 | 1.00E−04 | 4 | No | N/A |
| | 7050del | Val2351CysfsT | | VLVSSAASSQAEADKMS | | DRB1*13:02 | | | | | |
| | | er62 | | GTST | | | | | | | |
| 345 | NM_080866.2:c. | NP_543142.2:p. | 122737 | TLEILKSTMKKELEAAQ | 902 | HLA- | 134.77 | 1.00E−04 | 4 | No | N/A |
| | 1005del | Lys335AsnfsTer | | KKKPSLCEMLHMPNICK | | DRB1*13:02 | | | | | |
| | | 67 | | RISLLS | | | | | | | |
| 346 | NM_001304815. | NP_001291744. | 127222 | PAVPFSRSRQPSPLLLLPP | 1004 | HLA- | 404.57 | 1.00E−04 | 4 | No | N/A |
| | 1:c.2363del | 1:p.Pro788Leufs | | PAGLTSDPGPSVRRVPA | | DRB1*04:03 | | | | | |
| | | Ter4 | | VQRD | | | | | | | |
| 347 | NM_001291428. | NP_001278357. | 127395 | TGALLLQGFIQDRAGRM | 904 | HLA- | 120.58 | 1.00E−04 | 4 | No | N/A |
| | 1:c. 121del | 1 :p. Glu41 Argfs | | GGEAPELALDPVPQDAS | | DRB1*13:02 | | | | | |
| | | Ter19 | | TKKLSE | | | | | | | |
| 348 | NM_005357.2:c. | NP_005348.2:p. | 127907 | GAGPSGETGAAGVDGG | 907 | HLA- | 69.65 | 1.00E−04 | 4 | No | N/A |
| | 3222del | Arg 1075AspfsT | | CGGRH | | DRB1*13:02 | | | | | |
| | | er101 | | | | | | | | | |
| 349 | NM_004938.3:c. | NP_004929.2:p. | 131996 | DFFRAQTLKETSLTNTM | 910 | HLA- | 93.58 | 1.00E−04 | 4 | No | N/A |
| | 3822dup | Tyr1275ValfsTe | | GGYKESFSSIMCFGCHD | | DRB1*13:02 | | | | | |
| | | r64 | | VYSQAS | | | | | | | |
| 350 | NM_006617.1:c. | NP_006608.1:p. | 133962 | NQEFLQARTPTLASTPIP | 906 | HLA- | 136.44 | 1.00E−04 | 4 | No | N/A |
| | 1176del | Thr393HisfsTer | | PTPQAPSPAVDAEIRAQD | | DRB1*13:02 | | | | | |
| | | 9 | | APLS | | | | | | | |
| 351 | NM_032936.3:c. | NP_116325.1:p. | 138753 | GRCKSGFDPRHGSHNIK | 911 | HLA- | 83.36 | 1.28 | 4 | No | N/A |
| | 231del | Ala78ProfsTer1 | | KKAWYLIAMLLKLAFCL | | DRB1*13:01 | | | | | |
| | | 1 | | ALCAKL | | | | | | | |
| 352 | NM_001293228. | NP_001280157. | 189894 | PCFYPDEDDFYFGGPDST | 903 | HLA- | 615.3 | 1.00E−04 | 4 | No | N/A |
| | 1:c.134del | 1:p.Pro45 Argfs | | PPGEDIWKKFELLPTPPL | | DRB1*13:02 | | | | | |
| | | Ter86 | | SPSR | | | | | | | |
| 223 | NM_001318120. | NP_001305049. | 61 | DQLQQAVQSQGFINYCQ | 789 | HLA- | 14.7 | 24.08 | 3 | Tested | No |
| | 1:c.1384del | 1 :p.Ile462Leufs | | KKIDASQTEFEKNVWSF | | DRB1*13:01 | | | | | |
| | | Ter16 | | LKVNFE | | | | | | | |
| 240 | NM_001127698. | NP_001121170. | 499 | GNKCTMCKEKLEREAA | 779 | HLA- | 123.19 | 461.65 | 3 | Tested | No |
| | 1:c.2468del | 1:p.Lys823Argf | | EKKKKEDEDRSNTGERS | | DRB1*13:02 | | | | | |
| | | sTer119 | | NTGERSN | | | | | | | |
| 269 | NM_005379.3:c. | NP_005370.1:p. | 1360 | VKVVQGPAGGDNSKLR | 988 | HLA- | 39.78 | 72.67 | 3 | No | N/A |
| | 3098del | Lys1033ArgfsT | | YKKKGSHCLEVTVQ | | DRB1*13:01 | | | | | |
| | | er8 | | | | | | | | | |
| 290 | NM_001304717. | NP_001291646. | 2546 | CSIERADNDKEYLVLTLT | 822 | HLA- | 96.45 | 66.83 | 3 | Tested | No |
| | 2:c.1487del | 2:p.Asn496Metf | | KNDLDKANKDKANRYF | | DRB1*13:02 | | | | | |
| | | sTer21 | | SPNFKV | | | | | | | |
| 298 | NM_022167.2:c. | NP_071450.2:p. | 2949 | VNQEVLEILDFHLYGSYP | 859 | HLA- | 48.64 | 10.17 | 3 | No | N/A |
| | 1584del | Gly529AlafsTer | | PGTPALKAYWENTYDA | | DRB1*15:01 | | | | | |
| | | 78 | | ADGPSG | | | | | | | |
| 310 | NM_001321829. | NP_001308758. | 3796 | LSLEINRKLQAVLEDTLL | 920 | HLA- | 129.13 | 32.63 | 3 | No | N/A |
| | 1:c.2600del | 1:p.Asn867Ilefs | | KNITLKENLQTLGTEIER | | DRB1*03:01 | | | | | |
| | | Ter4 | | LIKH | | | | | | | |
| 353 | NM_001323890. | NP_001310819. | 5732 | GTMRATGDFVTVKDGEI | 919 | HLA- | 42.34 | 10.64 | 3 | No | N/A |
| | 1:c.1319del | 1 :p.Leu440Trpfs | | FFLGRKDSQIKRHGKRL | | DRB1*01:01 | | | | | |
| | | Ter43 | | NIELVQ | | | | | | | |
| 354 | NM_001243439. | NP_001230368. | 6292 | SFGSPTGNQMSSDIDEYK | 817 | HLA- | 186.11 | 25.32 | 3 | Tested | No |
| | 1:c.908del | 1:p.Asn303Thrf | | KNIHGNALRTSGSSSSDV | | DRB1*13:02 | | | | | |
| | | sTer63 | | TKAS | | | | | | | |
| 355 | NM_001256798. | NP_001243727. | 7641 | DGLRSRVKYGVKTTPES | 912 | HLA- | 17.13 | 4.6 | 3 | No | N/A |
| | 1:c.1128del | 1:p. Tyr377Thrfs | | PPYSSGSYDSIKTEVSGC | | DRB1*07:01 | | | | | |
| | | Ter20 | | PEDLT | | | | | | | |
| 356 | NM_006887.4:c. | NP_008818.3:p. | 7820 | GSAAAGGPTSYGTLKEP | 923 | HLA- | 306.58 | 118.56 | 3 | No | N/A |
| | 320del | Gly 107AlafsTer | | SGGGGTALLNKENKFRD | | DRB1*07:01 | | | | | |
| | | 80 | | RSFSEN | | | | | | | |
| 357 | NM_015477.2:c. | NP_056292.1:p. | 9724 | TAPSLQNNQPVEFNHAI | 916 | HLA- | 14.21 | 9.22 | 3 | No | N/A |
| | 931del | Val311LeufsTer | | NYVNKIKNRFQGQPDIY | | DRB1*01:01 | | | | | |
| | | 43 | | KAFLEI | | | | | | | |
| 358 | NM_001297647. | NP_001284576. | 10676 | REPAGLSLVLKKIPIPETP | 914 | HLA- | 81.74 | 42.13 | 3 | No | N/A |
| | 1:c.872del | 1 :p.Pro291Hisfs | | PQTPPQVLDSPHQRSPSL | | DRB1*13:01 | | | | | |
| | | Ter74 | | SLA | | | | | | | |
| 359 | NM_018284.2:c. | NP_060754.2:p. | 11328 | ESTQLQNEIQKLQKTLK | 1005 | HLA- | 64.94 | 44.84 | 3 | No | N/A |
| | 1753del | Thr585ProfsTer | | KKTKRYMSHKLKI | | DRB1*13:01 | | | | | |
| | | 9 | | | | | | | | | |
| 360 | NM_016441.2:c. | NP_057525.1:p. | 13140 | CTHCYCLQGQTLCSTVS | 918 | HLA- | 32.96 | 20.02 | 3 | No | N/A |
| | 2567del | Pro856LeufsTer | | CPPLPCVEPINVEGSCCP | | DRB1*13:02 | | | | | |
| | | 67 | | MCPEM | | | | | | | |
| 361 | NM_001008892. | NP_001008892. | 13172 | GLAMSSSIFIGGSFILKKK | 1006 | HLA- | 156.53 | 15.31 | 3 | No | N/A |
| | 2:c.101del | 1:p.Gly34Alafs | | GLLRLARKGSMRAGQG | | DRB1*07:01 | | | | | |
| | | Ter11 | | GHAYL | | | | | | | |
| 362 | NM_001305.3:c. | NP_001296.1:p. | 16973 | GASLYVGWAASGLLLL | 868 | HLA- | 266.76 | 19.67 | 3 | No | N/A |
| | 537del | Leu180CysfsTer | | GGGLLCCNCPPRTDKPY | | DRB1*13:02 | | | | | |
| | | 115 | | SAKYSAA | | | | | | | |
| 363 | NM_017895.7:c. | NP_060365.7:p. | 17486 | DLALRGKKKRKKFMKD | 1007 | HLA- | 161.06 | 16.52 | 3 | No | N/A |
| | 2072del | Lys691 ArgfsTer | | AKKKGEMTAEERSQFEI | | DRB1*13:01 | | | | | |
| | | 4 | | LKAQMFA | | | | | | | |
| 364 | NM_001134419. | NP_001127891. | 17738 | PMAFSPQRDRFQAEGSL | 921 | HLA- | 44.61 | 5.64 | 3 | No | N/A |
| | 1:c.92del | 1 :p. Asn31Thrfs | | KKNEQNFKLAGVKKDIE | | DRB1*01:02 | | | | | |
| | | Ter51 | | KLYEAV | | | | | | | |
| 365 | NM_001852.3:c. | NP_001843.1:p. | 26727 | PQGLPGVKGDKGSPGKT | 915 | HLA- | 124.25 | 52.16 | 3 | No | N/A |
| | 1312del | Arg438AlafsTer | | GPRGKVGDPGVAGLPGE | | DRB1*04:03 | | | | | |
| | | 93 | | KGEKGE | | | | | | | |
| 366 | NM_001330121. | NP_001317050. | 28356 | GAKIQWLKDAQGLPGG | 925 | HLA- | 77.87 | 37.29 | 3 | No | N/A |
| | 1:c.2839del | 1:p.Asp947Thrf | | GGGDNSGTAENGRHSDL | | DRB1*07:01 | | | | | |
| | | sTer41 | | AALYTIV | | | | | | | |
| 367 | NM_001256071. | NP_001243000. | 36945 | HKDAWRQPEDTWAALE | 798 | HLA- | 781.72 | 105.69 | 3 | No | N/A |
| | 2:c.2180_2182del | 2:p.Phe727del | | GLSFSPFREQMLDTSSLL | | DRB1*04:03 | | | | | |
| | | | | QFMREK | | | | | | | |
| 368 | NM_012334.2:c. | NP_036466.2:p. | 37857 | FRSKQEALKQGWLHKK | 927 | HLA- | 336.9 | 37.55 | 3 | No | N/A |
| | 3674dup | Ser1226LeufsTe | | GGGSSTLSRRNWKKRW | | DRB1*07:01 | | | | | |
| | | r25 | | FVLRQSKL | | | | | | | |
| 369 | NM_005523.5:c. | NP_005514.1:p. | 37912 | LTDRQVKIWFQNRRMK | 922 | HLA- | 300.53 | 11.57 | 3 | No | N/A |
| | 895del | Ile299LeufsTer3 | | EKKINRDRLQYYSANPL | | DRB1*07:01 | | | | | |
| | | 0 | | L | | | | | | | |
| 370 | NM_001031727. | NP_001026897. | 50720 | VRIAAPGIGVWNPAFDV | 926 | HLA- | 129.2 | 3.64 | 3 | No | N/A |
| | 2:c.988del | 1:p.His330Thrfs | | TPHDLITGGIITELGVFAP | | DRB1*07:01 | | | | | |
| | | Ter24 | | EELR | | | | | | | |
| 371 | NM_004615.3:c. | NP_004606.2:p. | 60397 | QNYTNWSTSPYFLEHGIP | 929 | HLA- | 39.06 | 27.73 | 3 | No | N/A |
| | 516del | Ser173AlafsTer | | PSCCMNETDCNPQDLHN | | DQA1*05:05 | | | | | |
| | | 4 | | LTVAA | | /DQB1*03:0 | | | | | |
| | | | | | | 1 | | | | | |
| 372 | NM_001278458. | NP_001265387. | 66370 | ESKFKSRASNAQAKPSSF | 897 | HLA- | 653.25 | 10.58 | 3 | No | N/A |
| | 1:c.2921del | 1 :p.Leu974Cysf | | FLQMQKRVSGHYVTSA | | DRB1*03:01 | | | | | |
| | | sTer12 | | AAKSVH | | | | | | | |
| 373 | NM_001084.4:c. | NP_001075.1:p. | 79942 | GCGFCNQDRRTLPGGQP | 932 | HLA- | 336.12 | 40.29 | 3 | No | N/A |
| | 889del | Arg297GlyfsTer | | PPRVFLAVFVEQPTPFLP | | DRB1*07:01 | | | | | |
| | | 61 | | RFLQR | | | | | | | |
| 374 | NM_001079935. | NP_001073404. | 90098 | MSYFPILFFFFLKRCPSYT | 937 | HLA- | 134.78 | 1.00E−04 | 3 | No | N/A |
| | 1:c.32del | 1:p.Phe11SerfsT | | EPQNLTGVSEFL | | DRB1*13:02 | | | | | |
| | | er89 | | | | | | | | | |
| 375 | NM_033305.2:c. | NP_150648.2:p. | 90735 | NLLPYKIAYYIEGIENSV | 1008 | HLA- | 54.06 | 1.00E−04 | 3 | No | N/A |
| | 6399_6400insTT | Thr2134PhefsTe | | FTLSEGHSAQICTAQLGK | | DRB1*01:01 | | | | | |
| | TTTTTT | r5 | | ARLH | | | | | | | |
| 376 | NM_080859.1:c. | NP_543135.1:p. | 96975 | DSCLLAAMAYDCYVAIR | 935 | HLA- | 113.57 | 1.00E−04 | 3 | No | N/A |
| | 391del | Leu131SerfsTer | | HPLPYATRMSRAMCAA | | DRB1*13:02 | | | | | |
| | | 56 | | LVGMAWL | | | | | | | |
| 377 | NM_139057.3:c. | NP_620688.2:p. | 103260 | QCYQEVCNDRINANTITS | 948 | HLA- | 140.09 | 1.00E−04 | 3 | No | N/A |
| | 3121del | Arg1041 AlafsT | | PRLAALTYKCTRDQWT | | DRB1*13:02 | | | | | |
| | | er5 | | VYCRVI | | | | | | | |
| 378 | NM_003500.3:c. | NP_003491.1:p. | 104064 | ARRGMHAFIVPIRSLQD | 960 | HLA- | 109.27 | 1.00E−04 | 3 | No | N/A |
| | 695del | Pro232HisfsTer | | HTPLPGIIIGDIGPKMDFD | | DRB1*04:03 | | | | | |
| | | 26 | | QTDN | | | | | | | |
| 379 | NM_002356.5:c. | NP_002347.5:p. | 104674 | PKAEDGATPSPSNETPKK | 888 | HLA- | 634.3 | 6 | 3 | No | N/A |
| | 464del | Lys 155ArgfsTer | | KKKRFSFKKSFKLSGFSF | | DRB1*15:01 | | | | | |
| | | 12 | | KKNK | | | | | | | |
| 380 | NM_001682.2:c. | NP_001673.2:p. | 104779 | AVVGIEDPVRPEVPDAIK | 943 | HLA- | 29.71 | 1.00E−04 | 3 | No | N/A |
| | 2083_2087dup | Cys697LysfsTer | | KCQRAGITVRMVTGDNI | | DRB1*13:02 | | | | | |
| | | 40 | | NTARA | | | | | | | |
| 381 | NM_001317948. | NP_001304877. | 105297 | IIIKCLLYARHGVLFLFFF | 949 | HLA- | 13.92 | 1.00E−04 | 3 | No | N/A |
| | 1:c.829dup | 1 :p. Ter277Leu | | | | DRB1*13:02 | | | | | |
| | | fsTer129 | | | | | | | | | |
| 382 | NM_001302819. | NP_001289748. | 109734 | DFLSVKWEAAMMNGKV | 931 | HLA- | 224.47 | 1.00E−04 | 3 | No | N/A |
| | 1:c. 149del | 1:p.Phe50SerfsT | | PFFFSSESLGYFATGRPA | | DRB1*13:02 | | | | | |
| | | er6 | | DNVMTT | | | | | | | |
| 383 | NM_006536.5:c. | NP_006527.1:p. | 112906 | AMDRNSLQSAVSNIAQA | 934 | HLA- | 202.31 | 1.00E−04 | 3 | No | N/A |
| | 2658_2659del | Phe887TyrfsTer | | PLFIPPNSDPVPARDYLIL | | DRB1*13:02 | | | | | |
| | | 6 | | KGVL | | | | | | | |
| 384 | NM_198517.3:c. | NP_940919.1:p. | 117439 | RGACPGLLETLGALRAIP | 954 | HLA- | 32.56 | 1.00E−04 | 3 | No | N/A |
| | 960del | Ala321 ArgfsTer | | PAQLQEEAFMSQVHSVV | | DRB1*13:02 | | | | | |
| | | 100 | | LSERD | | | | | | | |
| 385 | NM_001099439. | NP_001092909. | 120459 | MSGQDVIKAVEDGFRLP | 942 | HLA- | 91.92 | 1.00E−04 | 3 | No | N/A |
| | 1:c.2604del | 1:p.Arg869Glyf | | PPRNCPNLLHRLMLDCW | | DRB1*13:02 | | | | | |
| | | sTer10 | | QKDPGE | | | | | | | |
| 386 | NM_000783.3:c. | NP_000774.2:p. | 123484 | AGQGCKDALQLLIEHSW | 956 | HLA- | 39.95 | 1.00E−04 | 3 | No | N/A |
| | 843_847del | Gly282AlafsTer | | ERGERLDMQALKQSSTE | | DRB1*13:02 | | | | | |
| | | 50 | | LLFGGH | | | | | | | |
| 387 | NM_005456.3:c. | NP_005447.1:p. | 124050 | MAERESGGLGGGAASPP | 945 | HLA- | 96.9 | 1.00E−04 | 3 | No | N/A |
| | 37del | Ala13ProfsTer8 | | AASPFLGLHIASPPNF | | DRB1*04:03 | | | | | |
| | | 4 | | | | | | | | | |
| 388 | NM_001288608. | NP_001275537. | 131538 | SCGPGTQHRQLQCRQEF | 957 | HLA- | 13.97 | 1.00E−04 | 3 | No | N/A |
| | 1:c.2339del | 1:p.Gly780Valfs | | GGGGSSVPPERCGHLPR | | DRB1*01:01 | | | | | |
| | | Ter62 | | PNITQS | | | | | | | |
| 389 | NM_021191.2:c. | NP_067014.2:p. | 131598 | SSSLSSGHVHSTPFQAGT | 958 | HLA- | 284.5 | 1.00E−04 | 3 | No | N/A |
| | 910del | Arg304ValfsTer | | PRYDVPIDMSYDSYPHH | | DRB1*13:02 | | | | | |
| | | 6 | | GIGTQ | | | | | | | |
| 390 | NM_012326.2:c. | NP_036458.2:p. | 133272 | PTGPKNMQTSGRLSNVA | 930 | HLA- | 192.58 | 1.00E−04 | 3 | No | N/A |
| | 543del | Cys182AlafsTer | | PPCILRKNPPSARNGGHE | | DRB1*13:02 | | | | | |
| | | 31 | | TDAQI | | | | | | | |
| 391 | NM_001291415. | NP_001278344. | 134312 | MKSCGVSLATAAAAAA | 1009 | HLA- | 17.26 | 12.21 | 3 | No | N/A |
| | 1:c.33_38dup | 1:p.Ala16_Alal | | AFGDEEKKMAAGKASG | | DQA1*03:03 | | | | | |
| | | 7dup | | ESEEA | | /DQB1*03:0 | | | | | |
| | | | | | | 1 | | | | | |
| 392 | NM_203436.2:c. | NP_982260.2:p. | 138630 | RTAPLGVPGTLPGLPRR | 950 | HLA- | 218.85 | 1.00E−04 | 3 | No | N/A |
| | 109del | Leu37SerfsTer5 | | DPLRVALRLDAACWEW | | DRB1*04:03 | | | | | |
| | | 3 | | ARSGCAR | | | | | | | |
| 393 | NM_001080539. | NP_001074008. | 139682 | LRDSIQSAQELLAQEQK | 1010 | HLA- | 146.12 | 2.43 | 3 | No | N/A |
| | 1:c.849del | 1:p.Glu284Lysfs | | KKEELEIATSQLKSDLTS | | DRB1*13:01 | | | | | |
| | | Ter14 | | RDDLI | | | | | | | |
| 394 | NM_001330754. | NP_001317683. | 140003 | PGCLPMVKRTITRQQWK | 1011 | HLA- | 100.08 | 7.94 | 3 | No | N/A |
| | 1:c.912del | 1:p.Ala305Profs | | KKALRSMPKSRNQVLFR | | DRB1*11:04 | | | | | |
| | | Ter18 | | RNLTPS | | | | | | | |
| 395 | NM_194312.2:c. | NP_919288.2:p. | 140712 | EASASPPRSEAQRQIQEW | 1012 | HLA- | 153.14 | 1.00E−04 | 3 | No | N/A |
| | 1948del | Val650CysfsTer | | GVSVRTLRGNFESASGP | | DRB1*13:02 | | | | | |
| | | 35 | | LCGFN | | | | | | | |
| 396 | NM_001173464. | NP_001166935. | 141782 | GLPSKIGSISRQSSLSEKK | 944 | HLA- | 675.47 | 1.00E−04 | 3 | No | N/A |
| | 1:c.3703del | 1:p.Ile1235Phef | | IPEPSPVTRRKAYEKAEK | | DRB1*04:03 | | | | | |
| | | sTer7 | | SKA | | | | | | | |
| 397 | NM_001277075. | NP_001264004. | 142694 | SLRRHYEVHHGLCILKE | 953 | HLA- | 709.3 | 1.00E−04 | 3 | No | N/A |
| | 1:c.692del | 1 :p.Pro231 Argfs | | APPEEEACGDSPHAHES | | DRB1*13:02 | | | | | |
| | | Ter49 | | AGQPPP | | | | | | | |
| 398 | NM_152879.2:c. | NP_690618.2:p. | 142814 | QGIAVLNIPSYAGGTNF | 946 | HLA- | 111.35 | 1.00E−04 | 3 | No | N/A |
| | 2564del | Gly855ValfsTer | | WGGTKEDDTFAAPSFDD | | DRB1*13:02 | | | | | |
| | | 48 | | KILEVV | | | | | | | |
| 399 | NM_022162.1:c. | NP_071445.1:p. | 142896 | TTTDMYLLILQHFLLHA | 1013 | HLA- | 72.49 | 1.42 | 3 | No | N/A |
| | 1583del | Pro528GlnfsTer | | TPPDSASQGLGPSLLRGR | | DRB1*07:01 | | | | | |
| | | 235 | | LPTLL | | | | | | | |
| 400 | NM_001134382. | NP_001127854. | 143840 | RGKPPPQAHLPSAPALPP | 1014 | HLA- | 130.76 | 2.04 | 3 | No | N/A |
| | 2:c.2987del | 1:p.His996Thrfs | | PHPPVVLPHLQHSVAGH | | DQA1*05:01 | | | | | |
| | | Ter124 | | HLGPP | | /DQB1*03:0 | | | | | |
| | | | | | | 1 | | | | | |
| 401 | NM_001420.3:c. | NP_001411.2:p. | 144590 | MVTQILGAMESQVGGGP | 961 | HLA- | 307.53 | 1.00E−04 | 3 | No | N/A |
| | 47del | Gly16AlafsTer3 | | AGPALPNGPLLGTNGAT | | DRB1*04:03 | | | | | |
| | | 5 | | DD | | | | | | | |
| 402 | NM_004833.1:c. | NP_004824.1:p. | 145333 | LQLTSGVHSTIKVIKAKK | 1015 | HLA- | 253.48 | 1.13 | 3 | No | N/A |
| | 1027del | Thr343HisfsTer | | KT | | DRB1*07:01 | | | | | |
| | | 14 | | | | | | | | | |
| 403 | NM_025182.3:c. | NP_079458.2:p. | 149137 | VRQGALQGGLLMGYSP | 1016 | HLA- | 55.69 | 2.87 | 3 | No | N/A |
| | 124del | Ala42ArgfsTer2 | | AGGATSPGVYQVSIFSPP | | DRB1*01:01 | | | | | |
| | | 5 | | AGTSEP | | | | | | | |
| 404 | NM_003501.2:c. | NP_003492.2:p. | 149435 | ASTVEGGDTALLPEFPR | 959 | HLA- | 259.14 | 1.00E−04 | 3 | No | N/A |
| | 61del | Leu21 SerfsTer5 | | GPLDAYRARASFSWKEL | | DRB1*13:02 | | | | | |
| | | 7 | | ALFTEG | | | | | | | |

Supplementary Table 8. List of the Top 100 most immunogenic predicted MHC-I neoAgs,
with higher immunogenicity, obtained from the
computational methods in the validation set.

| | | | Chro- | | | Micro- | Reference | | Altered | Number | |
|---|---|---|---|---|---|---|---|---|---|---|---|
| Mutant | SEQ | | mo- | | | satel- | MS | | MS | deleted | |
| Epitope | ID | Gene | some | | | lite | Lengths | Variant | Length | nucleo- | Peptide |
| Sequence | NO | Name | | Start | Stop | motif | (repeats) | Type | (repeats) | tides | Length |
| KSAFATYKVK | 405 | TCF7L2 | chr10 | 114925316 | 114925317 | A | 9 | FS | 8 | −1 | 10 |
| NLHLKTTSL | 406 | TTK | chr6 | 80751896 | 80751897 | A | 9 | FS | 8 | −1 | 9 |
| FLRQRATSTI | 407 | EPC2 | chr2 | 149447828 | 149447829 | A | 8 | FS | 7 | −1 | 10 |
| LIKLKLNRL | 408 | ALMS1 | chr2 | 73800080 | 73800081 | A | 7 | FS | 6 | −1 | 9 |
| LMLLRLNL | 409 | SEC31A | chr4 | 83785564 | 83785565 | T | 9 | FS | 8 | −1 | 8 |
| HFINVMFVR | 410 | ZBTB41 | chr1 | 197145702 | 197145703 | T | 7 | FS | 6 | −1 | 9 |
| SALVRLFPV | 411 | FBXO34 | chr14 | 55818554 | 55818554 | T | 8 | FS | 7 | 1 | 9 |
| FPKKKCTNL | 412 | SGO1 | chr3 | 20216067 | 20216068 | T | 7 | FS | 6 | −1 | 9 |
| MSVKKVMTY | 413 | TGS1 | chr8 | 56711598 | 56711599 | A | 6 | FS | 5 | −1 | 9 |
| FATYKAKMPL | 414 | C5orf42 | chr5 | 37182874 | 37182875 | T | 6 | FS | 5 | −1 | 10 |
| MTLSKMIKK | 415 | RNPC3 | chr1 | 104076466 | 104076467 | A | 12 | FS | 11 | −1 | 9 |
| NPRRKTWKM | 416 | THAP5 | chr7 | 108205525 | 108205526 | T | 9 | FS | 8 | −1 | 9 |
| SQKKRRYSI | 417 | ZC3H13 | chr13 | 46543501 | 46543501 | A | 6 | FS | 7 | 1 | 9 |
| KTTNHSSQM | 418 | MIS18BP1 | chr14 | 45716018 | 45716019 | T | 11 | FS | 10 | −1 | 9 |
| AAICTTPAL | 419 | RNF103 | chr2 | 86831014 | 86831014 | A | 8 | FS | 9 | 1 | 9 |
| TATETKTPY | 420 | AP4E1 | chr15 | 51293338 | 51293340 | CT | 2 | FS | 1 | −1 | 9 |
| RIKKKLMEL | 421 | RPF2 | chr6 | 111346773 | 111346773 | A | 8 | FS | 9 | 1 | 9 |
| HSYHLLQAY | 422 | SETD5 | chr3 | 9486784 | 9486785 | A | 6 | FS | 5 | −1 | 9 |
| LSSVSFFLY | 423 | SLC16A4 | chr1 | 110906426 | 110906427 | A | 9 | FS | NA | −1 | 9 |
| KRATFLLAL | 424 | MSH3 | chr5 | 79970914 | 79970915 | A | 8 | FS | 7 | −1 | 9 |
| YYYGGNCGLFY | 425 | RNF128 | chrX | 106016280 | 106016280 | T | 7 | FS | 8 | 1 | 11 |
| LTMKEAVPK | 426 | CDH1 | chr16 | 68867215 | 68867216 | C | 4 | FS | 3 | −1 | 9 |
| HSASNGTPL | 189 | ZBTB20 | chr3 | 114058002 | 114058003 | G | 7 | FS | 6 | −1 | 9 |
| TQLARFFPI | 24 | RNF43 | chr17 | 56435160 | 56435161 | G | 7 | FS | 8 | −1 | 9 |
| AEISSQVPHW | 427 | MSANTD2 | chr11 | 124637730 | 124637730 | C | 4 | FS | Ins A | 1 | 10 |
| AEKCLILVW | 428 | MIA3 | chr1 | 222803533 | 222803534 | A | 6 | FS | 5 | −1 | 9 |
| TELGILTSF | 429 | YLPM1 | chr14 | 75283720 | 75283721 | T | 6 | FS | 5 | −1 | 9 |
| SLRRKYLRV | 430 | LMAN1 | chr18 | 57013193 | 57013194 | A | 9 | FS | 8 | −1 | 9 |
| SQFLTEGIMK | 431 | MET | chr7 | 116418873 | 116418876 | CAT | 2 | FS | 1 | −2 | 10 |
| LPLLRHHLPL | 432 | PPRC1 | chr10 | 103907123 | 103907124 | C | 13 | FS | 12 | −1 | 10 |
| FSDSEGEGL | 433 | REST | chr4 | 57777065 | 57777068 | AAG | 4 | FS | 3 | −2 | 9 |
| ITIGVRPIR | 434 | TNRC6A | chr16 | 24802365 | 24802366 | G | 7 | FS | 6 | −1 | 9 |
| YADQWTVL | 435 | STAT5A | chr17 | 40461419 | 40461420 | G | 5 | FS | 4 | −1 | 8 |
| HTMWHCALEK | 436 | KIAAO100 | chr17 | 26971122 | 26971123 | T | 7 | FS | 6 | −1 | 10 |
| SAKKRASV | 437 | SLC3A2 | chr11 | 62649528 | 62649529 | A | 8 | FS | 7 | −1 | 8 |
| VVQKVAWFYK | 438 | SLC28A2 | chr15 | 45558287 | 45558288 | T | 5 | FS | 4 | −1 | 10 |
| HPSGGPIPL | 439 | CD44 | chr11 | 35236424 | 35236425 | G | 6 | FS | 5 | −1 | 9 |
| KTVDHKVERK | 440 | ITGA6 | chr2 | 173368930 | 173368930 | A | 8 | FS | 9 | 1 | 10 |
| TYTICVTMPY | 441 | ATM | chr11 | 108121536 | 108121536 | G | 4 | FS | 5 | 1 | 10 |
| TLRKKKQTV | 442 | ARHGAP18 | chr6 | 129959602 | 129959602 | A | 8 | FS | 9 | 1 | 9 |
| EEKMYYLFVY | 443 | RNF111 | chr15 | 59384790 | 59384790 | A | 5 | FS | 6 | 1 | 10 |
| AENEKKILY | 444 | SLC39A10 | chr2 | 196544941 | 196544941 | A | 8 | FS | 9 | 1 | 9 |
| HSPTPTSAL | 445 | MED15 | chr22 | 20936975 | 20936976 | C | 13 | FS | 12 | −1 | 9 |
| AEQEQEVVAW | 446 | MRPL54 | chr19 | 3767280 | 3767280 | C | 4 | FS | 5 | 1 | 10 |
| WPIQRCACSV | 447 | FBXO34 | chr14 | 55817785 | 55817785 | A | 6 | FS | 7 | 1 | 10 |
| HQKRRKKNL | 448 | ANTXR2 | chr4 | 80905989 | 80905990 | C | 8 | FS | 7 | −1 | 9 |
| TLKTLFHLR | 449 | CDC16 | chr13 | 115037719 | 115037720 | A | 6 | FS | 5 | −1 | 9 |
| KSFTKNHSSK | 450 | ZNF585B | chr19 | 37676332 | 37676333 | A | 6 | FS | 5 | −1 | 10 |
| MIKNRISPL | 451 | SYNE2 | chr14 | 64450477 | 64450478 | A | 7 | FS | 6 | −1 | 9 |
| NIKKKPGTSL | 452 | TMEM60 | chr7 | 77423459 | 77423460 | A | 9 | FS | 8 | −1 | 10 |
| AESLRENFSW | 453 | PIK3C2A | chr11 | 17111375 | 17111376 | T | 6 | FS | 5 | −1 | 10 |
| TSVGLAWRW | 454 | C1RL | chr12 | 7249399 | 7249400 | C | 5 | FS | 4 | −1 | 9 |
| CLSLRKKAL | 455 | MYO3B | chr2 | 171509586 | 171509587 | T | 7 | FS | 6 | −1 | 9 |
| VIIKKNPALPK | 456 | SACS | chr13 | 23912863 | 23912864 | A | 9 | FS | 8 | −1 | 11 |
| GALPRIHNM | 457 | KIAA1211 | chr4 | 57179502 | 57179503 | A | 7 | FS | 6 | −1 | 9 |
| AESGPFRPGW | 458 | ABCC1 | chr16 | 16142119 | 16142119 | C | 6 | FS | 7 | 1 | 10 |
| TSLARPPPL | 459 | FOXP4 | chr6 | 41555084 | 41555085 | C | 5 | FS | 4 | −1 | 9 |
| TPESPPLQLW | 460 | NOL4L | chr20 | 31041555 | 31041555 | C | 7 | FS | 8 | 1 | 10 |
| KALADPSAF | 461 | RPLPO | chr12 | 120634723 | 120634726 | CCT | 2 | FS | 1 | −2 | 9 |
| KRKSILLHL | 462 | SREK1IP1 | chr5 | 64020297 | 64020298 | A | 7 | FS | 6 | −1 | 9 |
| YSTEKRKKY | 463 | FAM171B | chr2 | 187625934 | 187625934 | T | 6 | FS | 7 | 1 | 9 |
| RRRPRLPTL | 464 | NFIC | chr19 | 3453852 | 3453853 | C | 6 | FS | 5 | −1 | 9 |
| AMQDFFSYY | 465 | UBA5 | chr3 | 132394148 | 132394148 | T | 7 | FS | 8 | 1 | 9 |
| TEVTWWALRK | 466 | ITGB4 | chr17 | 73733649 | 73733650 | C | 5 | FS | 4 | −1 | 10 |
| CTACHTALGR | 467 | ARID1A | chr1 | 27100175 | 27100176 | C | 6 | FS | 5 | −1 | 10 |
| IEFRIKFLF | 468 | MBD4 | chr3 | 129155547 | 129155547 | A | 10 | FS | 11 | 1 | 9 |
| ITMQQIAVL | 469 | ZMAT3 | chr3 | 178748779 | 178748780 | A | 5 | FS | 4 | −1 | 9 |
| CLRPHRVQL | 470 | C7orf50 | chr7 | 1037310 | 1037311 | C | 7 | FS | 6 | −1 | 9 |
| KTHPIRTSL | 471 | AHCTF1 | chr1 | 247007130 | 247007131 | T | 7 | FS | 6 | −1 | 9 |
| HAVGCPVQM | 472 | RNF145 | chr5 | 158630629 | 158630631 | A | 9 | FS | 11 | −1 | 9 |
| TALVPPPAL | 473 | KIAA1549 | chr7 | 138602055 | 138602055 | C | 6 | FS | 8 | 1 | 9 |
| WIIPFWFPF | 474 | ZRANB2 | chr1 | 71532486 | 71532486 | A | 7 | FS | 8 | 1 | 9 |
| FTVITYFLW | 475 | SLC46A3 | chr13 | 29287060 | 29287061 | T | 7 | FS | 6 | −1 | 9 |
| LFLVREVQR | 476 | CHD1 | chr5 | 98206408 | 98206409 | A | 7 | FS | 6 | −1 | 9 |
| SSPPFHYPF | 477 | ZNF800 | chr7 | 127014127 | 127014127 | C | 5 | FS | 6 | 1 | 9 |
| RLHCQHSSL | 478 | TCF7L2 | chr10 | 114849205 | 114849205 | C | 7 | FS | 8 | 1 | 9 |
| NSKKTNATF | 479 | KDM4C | chr9 | 7170005 | 7170006 | A | 7 | FS | ins G | −1 | 9 |
| TKKRKMQSL | 480 | BEND5 | chr1 | 49201966 | 49201967 | A | 9 | FS | 8 | −1 | 9 |
| DLVPSCHPR | 481 | RNF43 | chr17 | 56436054 | 56436055 | NA | NA | FS | NA | −1 | 9 |
| KMQFRLLVLL | 482 | ANKRD12 | chr18 | 9257835 | 9257836 | A | 7 | FS | 6 | −1 | 10 |
| TLKRRTLAM | 483 | SAMD9 | chr7 | 92732337 | 92732338 | A | 5 | FS | 4 | −1 | 9 |
| TPALREYTM | 484 | NRIP1 | chr21 | 16338966 | 16338967 | T | 7 | FS | 6 | −1 | 9 |
| ATGLNIWKLK | 485 | NEPR0 | chr3 | 112727097 | 112727098 | A | 6 | FS | 5 | −1 | 10 |
| TTTCPSRPL | 486 | RBM33 | chr7 | 155531072 | 155531072 | CA | 6 | FS | 7 | 1 | 9 |
| FGMMSMASR | 487 | PRMT3 | chr11 | 20483595 | 20483596 | T | 5 | FS | 4 | −1 | 9 |
| FALCGFWQI | 488 | SLC35F5 | chr2 | 114500276 | 114500277 | T | 10 | FS | 9 | −1 | 9 |
| QVYSVPHFFF | 489 | KIAAO391 | chr14 | 35592699 | 35592699 | T | 9 | FS | 10 | 1 | 10 |
| SAASYLWPSR | 490 | FOXN3 | chr14 | 89878533 | 89878534 | C | 7 | FS | 6 | −1 | 10 |
| QLKKKHLKA | 491 | B3GLCT | chr13 | 31803391 | 31803391 | A | 8 | FS | 9 | 1 | 9 |
| GAKKHFGSF | 492 | SBNO1 | chr12 | 123814990 | 123814990 | A | 6 | FS | 7 | 1 | 9 |
| FPRSQHQSPL | 493 | KMT2B | chr19 | 36211898 | 36211899 | C | 7 | FS | 6 | −1 | 10 |
| STAAPPAPR | 494 | CDC42EP1 | chr22 | 37962638 | 37962639 | C | 7 | FS | 6 | −1 | 9 |
| YLFEGAQTV | 495 | SLC39A8 | chr4 | 103189224 | 103189224 | A | 7 | FS | 8 | 1 | 9 |
| TVLRVDQIMAK | 496 | CCT8 | chr21 | 30428865 | 30428868 | CAT | 2 | FS | 1 | −2 | 11 |
| ELSICIRIPR | 497 | GMDS | chr6 | 2245583 | 2245583 | NA | NA | FS | NA | 4 | 10 |
| KTYTCAITTVK | 498 | SEC63 | chr6 | 108214773 | 108214775 | T | 9 | FS | 7 | −1 | 11 |
| WLEKKNCYSL | 499 | RMDN3 | chr15 | 41029893 | 41029893 | A | 9 | FS | 10 | 1 | 10 |
| RSVLWERVV | 500 | SLFN13 | chr17 | 33771843 | 33771843 | T | 5 | FS | 6 | 1 | 9 |
| HLLQEYLPL | 501 | YLPM1 | chr14 | 75248387 | 75248388 | C | 5 | FS | 4 | −1 | 9 |
| CPSPGPPSL | 502 | CLSTN3 | chr12 | 7310657 | 7310658 | C | 7 | FS | 6 | −1 | 9 |

| | | | | | | | | | | Pre- | | |
|---|---|---|---|---|---|---|---|---|---|---|---|---|
| | | | | | | | Bind- | | | dicted | | |
| | | | | | | | ing | | | in | | |
| | | | | Wild- | | | Af- | Tumor | | the | Eli- | Eli- |
| SEQ | | | Immuno- | type | SEQ | | fin- | Abun- | Sample | Dis- | spot | spot |
| ID | | | genicity | se- | ID | HLA | ity | dance | Recur- | covery | test- | reac- |
| NO | HGVSc | HGVSp | Score | quence | NO | Allele | (nM) | (TPM) | rence | Set | ed | tive |
| 405 | NM_001146274. | NP_001139746. | 1 | ALFGL | 788 | HLA- | 29.5 | 1476.901717 | 1 | No | No | NA |
| | 1:c.1403de1 | 1:p.Lys468Ser | | DRQTL | | A*11: | | | | | | |
| | | fsTer23 | | WCKPC | | 01 | | | | | | |
| | | | | RRKKK | | | | | | | | |
| | | | | CVRYI | | | | | | | | |
| | | | | QGEGS | | | | | | | | |
| | | | | CLSPP | | | | | | | | |
| | | | | SSDGS | | | | | | | | |
| 406 | NM_003318.4: | NP_003309.2: | 2 | HYSGG | 1017 | HLA- | 7.54 | 75.69082705 | 2 | No | No | NA |
| | c.2560del | p.Arg854Gly | | ESHNS | | B*08: | | | | | | |
| | | fsTer39 | | SSSKT | | 01 | | | | | | |
| | | | | FEKKR | | | | | | | | |
| | | | | GKK | | | | | | | | |
| 407 | NM_015630.3: | NP_056445.3: | 3 | SEHHL | 1018 | HLA- | 19.4 | 90.76268639 | 1 | No | No | NA |
| | c.207del | pGlu70Arg | | QRAIS | | B*08: | | | | | | |
| | | fsTer39 | | AQQVF | | 01 | | | | | | |
| | | | | REKKE | | | | | | | | |
| | | | | SMVIP | | | | | | | | |
| | | | | VPEAE | | | | | | | | |
| | | | | SNVNY | | | | | | | | |
| | | | | YNRLY | | | | | | | | |
| 408 | NM_015120.4: | NP_055935.4: | 6 | KKRFK | 1019 | HLA- | 25.7 | 244.2464458 | 1 | No | No | NA |
| | c.11080del | p.Ser3696Ala | | SLEKS | | B*08: | | | | | | |
| | | fsTer27 | | HKNTG | | 01 | | | | | | |
| | | | | ELKKS | | | | | | | | |
| | | | | KVLSH | | | | | | | | |
| | | | | HRAGR | | | | | | | | |
| | | | | SNQIK | | | | | | | | |
| | | | | IEQIK | | | | | | | | |
| 409 | NM_001318120.1: | NP_001305049.1: | 7 | DQLQQ | 789 | HLA- | 55.29 | 113.4902951 | 2 | Yes | No | NA |
| | c.1384de1 | p.Ile462Leu | | AVQSQ | | B*08: | | | | | | |
| | | fsTer16 | | GFINY | | 01 | | | | | | |
| | | | | CQKKI | | | | | | | | |
| | | | | DASQT | | | | | | | | |
| | | | | EFEKN | | | | | | | | |
| | | | | VWSFL | | | | | | | | |
| | | | | KVNFE | | | | | | | | |
| 410 | NM_194314.2: | NP_919290.2: | 8 | CDECG | 1020 | HLA- | 8.2 | 117.8015983 | 1 | No | No | NA |
| | c.1870del | p.Ile624Tyr | | KTFIR | | A*33: | | | | | | |
| | | fsTer88 | | HDHLT | | 01 | | | | | | |
| | | | | KHKKI | | | | | | | | |
| | | | | HSGEK | | | | | | | | |
| | | | | AHQCE | | | | | | | | |
| | | | | ECGKC | | | | | | | | |
| | | | | FGRRD | | | | | | | | |
| 411 | NM_017943.3: | NP_060413.2: | 10 | DQPSI | 1021 | HLA- | 25 | 23.76940308 | 1 | No | No | NA |
| | c.1454dup | p.Leu485Phe | | LNSCE | | C*16: | | | | | | |
| | | fsTer15 | | DPVPG | | 01 | | | | | | |
| | | | | MLFFL | | | | | | | | |
| | | | | PPGQH | | | | | | | | |
| | | | | LSDYS | | | | | | | | |
| | | | | QLNES | | | | | | | | |
| | | | | TTKES | | | | | | | | |
| 412 | NM_0011992 | NP_0011861811: | 12 | RRKSK | 1022 | HLA- | 9.49 | 30.61283787 | 1 | No | No | NA |
| | 52.1: | p.Thr319Leu | | RMSKY | | B*08: | | | | | | |
| | c.955del | fsTer34 | | KENKS | | 01 | | | | | | |
| | | | | ENKKT | | | | | | | | |
| | | | | VPQKK | | | | | | | | |
| | | | | MHKSV | | | | | | | | |
| | | | | SSNDA | | | | | | | | |
| | | | | YNFNL | | | | | | | | |
| 413 | NM_024831.6: | NP_079107.6: | 14 | VHDAS | 1023 | HLA- | 9.33 | 96.32369965 | 1 | No | No | NA |
| | c.1674del | pGly559Val | | TSSDS | | C*16: | | | | | | |
| | | fsTer35 | | EEQDM | | 01 | | | | | | |
| | | | | SVKKG | | | | | | | | |
| | | | | DDLLE | | | | | | | | |
| | | | | TNNPE | | | | | | | | |
| | | | | PEKCQ | | | | | | | | |
| | | | | SVSSA | | | | | | | | |
| 414 | NM_023073.3: | NP_075561.3: | 23 | ILTSL | 1024 | HLA- | 30.47 | 169.4167381 | 1 | No | No | NA |
| | c.5408del | p.Asn1803Met | | WLLEQ | | C*16: | | | | | | |
| | | fsTer7 | | PYFAT | | 01 | | | | | | |
| | | | | YKAKN | | | | | | | | |
| | | | | AIIKM | | | | | | | | |
| | | | | VENRD | | | | | | | | |
| | | | | TGCQI | | | | | | | | |
| | | | | GPNIE | | | | | | | | |
| 415 | NM_017619.3: | NP_060089.1: | 25 | KEQDR | 1025 | HLA- | 7.94 | 3.364743162 | 1 | No | No | NA |
| | c.358del | pArg120Gly | | VHSPC | | A*11: | | | | | | |
| | | fsTer18 | | PTSGS | | 01 | | | | | | |
| | | | | EKKKR | | | | | | | | |
| | | | | SDDPV | | | | | | | | |
| | | | | EDDKE | | | | | | | | |
| | | | | KKELG | | | | | | | | |
| | | | | YLTVE | | | | | | | | |
| 416 | NM_001130475.1: | NP_001123947.1: | 31 | VPTIF | 1026 | HLA- | 23.77 | 43.93258991 | 1 | No | No | NA |
| | c.297del | p.Lys99Asn | | SLPED | | B*08: | | | | | | |
| | | fsTer25 | | NQGKD | | 01 | | | | | | |
| | | | | PSKKK | | | | | | | | |
| | | | | SQKKN | | | | | | | | |
| | | | | LEDEK | | | | | | | | |
| | | | | EVCPK | | | | | | | | |
| | | | | AKSEE | | | | | | | | |
| 417 | NM_001330564.1: | NP_001317493.1: | 32 | ELVEM | 1027 | HLA- | 12.32 | 12.49563374 | 1 | No | No | NA |
| | c.3177dup | p.Glu1060Arg | | CNGKN | | B*08: | | | | | | |
| | | fsTer7 | | GILED | | 01 | | | | | | |
| | | | | SQKKE | | | | | | | | |
| | | | | DTAFS | | | | | | | | |
| | | | | DWSDE | | | | | | | | |
| | | | | DVPDR | | | | | | | | |
| | | | | TEVTE | | | | | | | | |
| 418 | NM_018353.4: | NP_060823.3: | 36 | EPQKS | 1028 | HLA- | 85.58 | 149.0310963 | 1 | Yes | No | NA |
| | c.471del | p.Lys157Asn | | GNNET | | C*16: | | | | | | |
| | | fsTer24 | | FTPNR | | 01 | | | | | | |
| | | | | VEKKK | | | | | | | | |
| | | | | LQHTY | | | | | | | | |
| | | | | LCEEK | | | | | | | | |
| | | | | ENNKS | | | | | | | | |
| | | | | FQSDD | | | | | | | | |
| 419 | NM_005667.3: | NP_005658.1: | 37 | LAGGR | 1029 | HLA- | 15.65 | 4.998220761 | 1 | No | No | NA |
| | c.2009dup | p.Gln671Ala | | HCCPV | | C*16: | | | | | | |
| | | fsTer12 | | CRWPS | | 01 | | | | | | |
| | | | | YKKKQ | | | | | | | | |
| | | | | PYAQH | | | | | | | | |
| | | | | QPLSN | | | | | | | | |
| | | | | DVPS | | | | | | | | |
| 420 | NM_007347.4: | NP_031373.2: | 38 | KQNVK | 1030 | HLA- | 26.52 | 47.59124794 | 1 | No | No | NA |
| | c.3214_3215del | p.Leu1072Ala | | MSESQ | | C*16: | | | | | | |
| | | fsTer10 | | AALPS | | 01 | | | | | | |
| | | | | ALKTL | | | | | | | | |
| | | | | QQKLR | | | | | | | | |
| | | | | LHIIE | | | | | | | | |
| | | | | IIGNE | | | | | | | | |
| | | | | GLLAC | | | | | | | | |
| 421 | NM_032194.1: | NP_115570.1: | 40 | RPAER | 1031 | HLA- | 22.05 | 2.396696541 | 1 | No | No | NA |
| | c.917dup | p.Asn306LysfsT | | ITEDH | | -B*08 | | | | | | |
| | | Er? | | EKKSK | | :01 | | | | | | |
| | | | | RI | | | | | | | | |
| | | | | KKN | | | | | | | | |
| 422 | NM_001080517.2: | NP_001073986.1: | 42 | NYKVD | 1032 | HLA- | 11.33 | 117.8533251 | 2 | No | No | NA |
| | c.1246de1 | p.Arg416Gly | | CACHK | | C*12: | | | | | | |
| | | fsTer34 | | GNRNC | | 03 | | | | | | |
| | | | | PIQKR | | | | | | | | |
| | | | | NPNAT | | | | | | | | |
| | | | | ELPLL | | | | | | | | |
| | | | | PPPPS | | | | | | | | |
| | | | | LPTIG | | | | | | | | |
| 423 | NM_004696.2: | NP_004687.1: | 43 | NGSFY | 1033 | HLA- | 7.45 | 7.365770553 | 1 | No | No | NA |
| | c.1425del | p.Phe475Leu | | FSGIC | | A*29: | | | | | | |
| | | fsTer12 | | YLLSS | | 02 | | | | | | |
| | | | | VSFFF | | | | | | | | |
| | | | | VPLAE | | | | | | | | |
| | | | | RWKNS | | | | | | | | |
| | | | | LT | | | | | | | | |
| 424 | NM_002439.4: | NP_002430.3: | 44 | STSYL | 830 | HLA- | 54.4 | 53.93262102 | 2 | Yes | No | NA |
| | c.1148del | p.Lys383Arg | | LCISE | | C*07: | | | | | | |
| | | fsTer32 | | NKENV | | 01 | | | | | | |
| | | | | RDKKK | | | | | | | | |
| | | | | GNIFI | | | | | | | | |
| | | | | GIVGV | | | | | | | | |
| | | | | QPATG | | | | | | | | |
| | | | | EVVFD | | | | | | | | |
| 425 | NM_194463.1: | NP_919445.1: | 46 | VIEVG | 1034 | HLA- | 6.03 | 0.648212697 | 1 | No | No | NA |
| | c.629dup | p.Val211Arg | | KKHGP | | A*29: | | | | | | |
| | | fsTer42 | | WVNHY | | 02 | | | | | | |
| | | | | SIFFV | | | | | | | | |
| | | | | SVSFF | | | | | | | | |
| | | | | IITAA | | | | | | | | |
| | | | | TVGYF | | | | | | | | |
| | | | | IFYSA | | | | | | | | |
| 426 | NM_004360.3: | NP_004351.1: | 47 | PDEIG | 1035 | HLA- | 8.59 | 9.292722995 | 1 | No | No | NA |
| | c.2466del | p.Thr823Gln | | NFIDE | | A*11: | | | | | | |
| | | fsTer23 | | NLKAA | | 01 | | | | | | |
| | | | | DTDPT | | | | | | | | |
| | | | | APPYD | | | | | | | | |
| | | | | SLLVF | | | | | | | | |
| | | | | DYEGS | | | | | | | | |
| | | | | GSEAA | | | | | | | | |
| 189 | NM_001164342.2: | NP_001157814.1: | 53 | HKTLL | 951 | HLA- | 21.96 | 15.82316283 | 2 | Yes | No | NA |
| | c.2075de1 | p.Pro692Leu | | ERHVA | | C*16: | | | | | | |
| | | fsTer43 | | LHSAS | | 01 | | | | | | |
| | | | | NGTPP | | | | | | | | |
| | | | | AGTPP | | | | | | | | |
| | | | | GARAG | | | | | | | | |
| | | | | PPGVV | | | | | | | | |
| | | | | ACTEG | | | | | | | | |
| 24 | NM_017763.4: | NP_060233.3: | 61 | FNLQK | 784 | HLA- | 48.47 | 105.9103245 | 2 | Yes | Yes | Yes |
| | c.1976del | p.Gly659Val | | SSLSA | | B*08: | | | | | | |
| | | fsTer41 | | RHPQR | | 01 | | | | | | |
| | | | | KRRGG | | | | | | | | |
| | | | | PSEPT | | | | | | | | |
| | | | | PGSRP | | | | | | | | |
| | | | | QDATV | | | | | | | | |
| | | | | HPACQ | | | | | | | | |
| 427 | NM_001308027.1: | NP_001294956.1: | 63 | TRWKE | 1036 | HLA- | 7.17 | 10.08955472 | 1 | No | No | NA |
| | c.1021_1022insA | p.Leu341His | | DIRYH | | B*44: | | | | | | |
| | | fsTer12 | | YAEIS | | 03 | | | | | | |
| | | | | SQVPL | | | | | | | | |
| | | | | GKRLR | | | | | | | | |
| | | | | EYFNS | | | | | | | | |
| | | | | EKPEG | | | | | | | | |
| | | | | RIIMT | | | | | | | | |
| 428 | NM_001324062.1: | NP_001310991.1: | 64 | VLDKV | 1037 | HLA- | 11.87 | 32.4719058 | 1 | No | No | NA |
| | c.2977de1 | p.Met993Cys | | FRASE | | B*44: | | | | | | |
| | | fsTer14 | | SQILS | | 03 | | | | | | |
| | | | | IAEKM | | | | | | | | |
| | | | | LDTRV | | | | | | | | |
| | | | | AENRD | | | | | | | | |
| | | | | LGMNE | | | | | | | | |
| | | | | NNIFE | | | | | | | | |
| 429 | NM_019589.2: | NP_062535.2: | 65 | YRTSM | 1038 | HLA- | 77.56 | 34.88988455 | 1 | No | No | NA |
| | c.5778del | pPhe1928Ser | | FKTFK | | B*44: | | | | | | |
| | | fsTer33 | | KTLDD | | 03 | | | | | | |
| | | | | GFFPF | | | | | | | | |
| | | | | IILDA | | | | | | | | |
| | | | | INDRV | | | | | | | | |
| | | | | RHFDQ | | | | | | | | |
| | | | | FWSAA | | | | | | | | |
| 430 | NM_005570.3: | NP_005561.1: | 70 | KEKYQ | 1039 | HLA- | 34.46 | 6.122166967 | 1 | No | No | NA |
| | c.912del | p.Glu305Arg | | EEFEH | | B*08: | | | | | | |
| | | fsTer22 | | FQQEL | | 01 | | | | | | |
| | | | | DKKKE | | | | | | | | |
| | | | | EFQKG | | | | | | | | |
| | | | | HPDLQ | | | | | | | | |
| | | | | GQPAE | | | | | | | | |
| | | | | EIFES | | | | | | | | |
| 431 | NM_001127500.2: | NP_0011209721: | 71 | SLNRI | 1040 | HLA- | 70.85 | 58.26789883 | 1 | No | No | NA |
| | c.3444_3446del | p.Ile1148del | | TDIGE | | A*11: | | | | | | |
| | | | | VSQFL | | 01 | | | | | | |
| | | | | TEGII | | | | | | | | |
| | | | | MKDFS | | | | | | | | |
| | | | | HPNVL | | | | | | | | |
| | | | | SLLGI | | | | | | | | |
| | | | | CLRSE | | | | | | | | |
| 432 | NM_015062.3: | NP_055877.3: | 72 | ASSSS | 1041 | HLA- | 14.16 | 13.85913686 | 1 | Yes | No | NA |
| | c.4375del | p.Ser1459Pro | | SSSSS | | B*08: | | | | | | |
| | | fsTer81 | | SSRSR | | 01 | | | | | | |
| | | | | SRSLS | | | | | | | | |
| | | | | PPHKR | | | | | | | | |
| | | | | WRRSS | | | | | | | | |
| | | | | CSSSG | | | | | | | | |
| | | | | RSRRC | | | | | | | | |
| 433 | NM_005612.4: | NP_005603.3: | 75 | ERQMA | 1042 | HLA- | 27.97 | 54.50562555 | 2 | No | No | NA |
| | c.266_268del | pGlu89del | | ELMPV | | C*08: | | | | | | |
| | | | | GDNNF | | 02 | | | | | | |
| | | | | SDSEE | | | | | | | | |
| | | | | GEGLE | | | | | | | | |
| | | | | ESADI | | | | | | | | |
| | | | | KGEPH | | | | | | | | |
| | | | | GLENM | | | | | | | | |
| 434 | NM_014494.2: | NP_055309.2: | 76 | DPKPA | 1043 | HLA- | 28.13 | 12.55742051 | 1 | No | No | NA |
| | c.2409del | p.Trp804Gly | | LRWGD | | A*68: | | | | | | |
| | | fsTer99 | | SKGSN | | 01 | | | | | | |
| | | | | CQGGW | | | | | | | | |
| | | | | EDDSA | | | | | | | | |
| | | | | ATGMV | | | | | | | | |
| | | | | KSNQW | | | | | | | | |
| | | | | GNCKE | | | | | | | | |
| 435 | NM_001288718.1: | NP_001275647.1: | 77 | KPQIK | 1044 | HLA- | 16.97 | 5.537065595 | 2 | No | No | NA |
| | c.2144de1 | p.Gly715Ala | | QVVPE | | C*08: | | | | | | |
| | | fsTer101 | | FVNAS | | 02 | | | | | | |
| | | | | ADAGG | | | | | | | | |
| | | | | SSATY | | | | | | | | |
| | | | | MDQAP | | | | | | | | |
| | | | | SPAVC | | | | | | | | |
| | | | | PQAPY | | | | | | | | |
| 436 | NM_014680.3: | NP_055495.2: | 83 | KWCQR | 1045 | HLA- | 7.74 | 69.33014247 | 1 | No | No | NA |
| | c.151del | p.Trp51Gly | | KLQAE | | A*11: | | | | | | |
| | | fsTer77 | | LKIGS | | 01 | | | | | | |
| | | | | FRFFW | | | | | | | | |
| | | | | IQNVS | | | | | | | | |
| | | | | LKFQQ | | | | | | | | |
| | | | | HQQTV | | | | | | | | |
| | | | | EIDNL | | | | | | | | |
| 437 | NM_001012662.2: | NP_001012680.1: | 87 | DPNFG | 1046 | HLA- | 81.71 | 18.29583114 | 1 | No | No | NA |
| | c.902del | p.Lys301Arg | | SKEDF | | B*08: | | | | | | |
| | | fsTer31 | | DSLLQ | | 01 | | | | | | |
| | | | | SAKKK | | | | | | | | |
| | | | | SIRVI | | | | | | | | |
| | | | | LDLTP | | | | | | | | |
| | | | | NYRGE | | | | | | | | |
| | | | | NSWFS | | | | | | | | |
| 438 | NM_004212.3: | NP_004203.2: | 93 | SILYY | 1047 | HLA- | 10.72 | 2.268710501 | 1 | No | No | NA |
| | c.875del | p.Leu292Tyr | | LGLVQ | | A*11: | | | | | | |
| | | fsTer23 | | WVVQK | | 01 | | | | | | |
| | | | | VAWFL | | | | | | | | |
| | | | | QITMG | | | | | | | | |
| | | | | TTATE | | | | | | | | |
| | | | | TLAVA | | | | | | | | |
| | | | | GNIFV | | | | | | | | |
| 439 | NM_000610.3: | NP_000601.3: | 95 | SNVNR | 1048 | HLA- | 34.57 | 251.8292447 | 1 | No | No | NA |
| | c.1842del | p.Ser615Pro | | SLSGD | | B*35: | | | | | | |
| | | fsTer28 | | QDTFH | | 03 | | | | | | |
| | | | | PSGGS | | | | | | | | |
| | | | | HTTHG | | | | | | | | |
| | | | | SESDG | | | | | | | | |
| | | | | HSH | | | | | | | | |
| | | | | GSQEG | | | | | | | | |
| | | | | GA | | | | | | | | |
| `440 | NM_001079818.1: | NP_0010732861: | 97 | RKEER | 1049 | HLA- | 63.11 | 11.19741262 | 1 | Yes | No | NA |
| | c.3234dup | p.Gln1079Thr | | EIKDE | | A*11: | | | | | | |
| | | fsTer10 | | KYIDN | | 01 | | | | | | |
| | | | | LEKKQ | | | | | | | | |
| | | | | WITKW | | | | | | | | |
| | | | | NENES | | | | | | | | |
| | | | | YS | | | | | | | | |
| 441 | NM_000051.3: | NP_000042.3: | 102 | ELSPL | 1050 | HLA- | 34.16 | 5.88459648 | 1 | No | No | NA |
| | c.1348dup | p.Glu450Gly | | LMILS | | A*68: | | | | | | |
| | | fsTer37 | | QLLPQ | | 01 | | | | | | |
| | | | | QRHGE | | | | | | | | |
| | | | | RTPYV | | | | | | | | |
| | | | | LRCLT | | | | | | | | |
| | | | | EVALC | | | | | | | | |
| | | | | QDKRS | | | | | | | | |
| 442 | NM_033515.2: | NP_277050.2: | 105 | QAAAV | 1051 | HLA- | 21.25 | 0.875597361 | 1 | No | No | NA |
| | c.488dup | p.Asn163Lys | | QKRVE | | B*08: | | | | | | |
| | | fsTer8 | | TVSQT | | 01 | | | | | | |
| | | | | LRKKN | | | | | | | | |
| | | | | KQYQI | | | | | | | | |
| | | | | PDVRD | | | | | | | | |
| | | | | IFAQQ | | | | | | | | |
| | | | | RESKE | | | | | | | | |
| 443 | NM_001330331.1: | NP_001317260.1: | 106 | KLHCK | 1052 | HLA- | 18.9 | 3.450552335 | 1 | No | No | NA |
| | c.2850dup | p.Cys951Met | | QDGEE | | B*44: | | | | | | |
| | | fsTer12 | | GTEED | | 03 | | | | | | |
| | | | | TEEKC | | | | | | | | |
| | | | | TICLS | | | | | | | | |
| | | | | ILEEG | | | | | | | | |
| | | | | EDVRR | | | | | | | | |
| | | | | LPCMH | | | | | | | | |
| 444 | NM_001127257.1: | NP_001120729.1: | 107 | MTELE | 1053 | HLA- | 30.89 | 10.11071098 | 1 | No | No | NA |
| | c.183dup | p.Tyr62Ile | | PSKFS | | B*44: | | | | | | |
| | | fsTer4 | | KQAAE | | 03 | | | | | | |
| | | | | NEKKY | | | | | | | | |
| | | | | YIEKL | | | | | | | | |
| | | | | FERYG | | | | | | | | |
| | | | | ENGRL | | | | | | | | |
| | | | | SFFGL | | | | | | | | |
| 445 | NM_001003891.1: | NP_001003891.1: | 112 | SSPSP | 1054 | HLA- | 86.37 | 4.47265384 | 1 | No | No | NA |
| | c.1357de1 | p.Gln453Ser | | GQQVQ | | C*16: | | | | | | |
| | | fsTer41 | | TPQSM | | 01 | | | | | | |
| | | | | PPPPQ | | | | | | | | |
| | | | | PSPQP | | | | | | | | |
| | | | | GQPSS | | | | | | | | |
| | | | | QPNSN | | | | | | | | |
| | | | | VSSGP | | | | | | | | |
| 446 | NM_172251.2: | NP_758455.1: | 114 | KPDAE | 1055 | HLA- | 17.34 | 0.803405824 | 1 | No | No | NA |
| | c.311dup | p.Thr106Asp | | YPEWL | | B*44: | | | | | | |
| | | fsTer42 | | FEMNL | | 03 | | | | | | |
| | | | | GPPKT | | | | | | | | |
| | | | | LEELD | | | | | | | | |
| | | | | PESRE | | | | | | | | |
| | | | | YWRRL | | | | | | | | |
| | | | | RKQNI | | | | | | | | |
| 447 | NM_017943.3: | NP_060413.2: | 115 | QMVAFL | 1056 | HLA-B*08: | 113.93 | 18.7306847 | 1 | No | No | NA |
| | c.683dup | p.Asn228Lys | | EQRASA | | 01 | | | | | | |
| | | fsTer23 | | LLASCS | | | | | | | | |
| | | | | KNCTNS | | | | | | | | |
| | | | | PAIVRE | | | | | | | | |
| | | | | SGQSRG | | | | | | | | |
| | | | | VPAV | | | | | | | | |
| 448 | NM_001145794.1: | NP_001139266.1: | 121 | WWFWPL | 1057 | HLA-B*08: | 87.74 | 16.01766224 | 1 | No | No | NA |
| | c.1069de1 | p.Ala357Pro | | CCKVVI | | 01 | | | | | | |
| | | fsTer52 | | KDPPPP | | | | | | | | |
| | | | | PAPAPK | | | | | | | | |
| | | | | EEEEEP | | | | | | | | |
| | | | | LPTKKW | | | | | | | | |
| | | | | PTVD | | | | | | | | |
| 449 | NM_001078645.2: | NP_001072113.1: | 122 | IKDKLK | 1058 | HLA-A*33: | 14.7 | 5.570944675 | 1 | No | No | NA |
| | c.1670de1 | p.Asn557Thr | | CYDFDV | | 01 | | | | | | |
| | | fsTer14 | | HTMKTL | | | | | | | | |
| | | | | KNIISP | | | | | | | | |
| | | | | PWDFRE | | | | | | | | |
| | | | | FEVEKQ | | | | | | | | |
| | | | | TAEE | | | | | | | | |
| 450 | NM_152279.3: | NP_689492.3: | 125 | IHTGEK | 1059 | HLA-A*11: | 72.36 | 26.87688773 | 2 | No | No | NA |
| | c.2106del | p.Lys702Asn | | PYECSD | | 01 | | | | | | |
| | | fsTer32 | | CGKSFT | | | | | | | | |
| | | | | KKSQLQ | | | | | | | | |
| | | | | VHQRIH | | | | | | | | |
| | | | | TGEKPY | | | | | | | | |
| | | | | VCAE | | | | | | | | |
| 451 | NM_182914.2: | NP_878918.2: | 126 | WRKLVS | 1060 | HLA-B*08: | 3.17 | 266.371014 | 1 | No | No | NA |
| | c.2031del | p.Lys677Asn | | KTQLEM | | 01 | | | | | | |
| | | fsTer30 | | NLPLMI | | | | | | | | |
| | | | | KKQDQP | | | | | | | | |
| | | | | TFDNSG | | | | | | | | |
| | | | | NILSKE | | | | | | | | |
| | | | | EKAT | | | | | | | | |
| 452 | NM_032936.3: | NP_116325.1: | 131 | GRCKSG | 911 | HLA-B*08: | 50 | 17.16693892 | 1 | No | No | NA |
| | c.231del | p.Ala78Pro | | FDPRHG | | 01 | | | | | | |
| | | fsTer11 | | SHNIKK | | | | | | | | |
| | | | | KAWYLI | | | | | | | | |
| | | | | AMLLKL | | | | | | | | |
| | | | | AFCLAL | | | | | | | | |
| | | | | CAKL | | | | | | | | |
| 453 | NM_001321378.1: | NP_001308307.1: | 134 | RQRELQ | 1061 | HLA-B*57: | 13.82 | 3.403730165 | 1 | No | No | NA |
| | c.4970de1 | p.Phe1657Ser | | LSVLSA | | 01 | | | | | | |
| | | fsTer5 | | ESLREN | | | | | | | | |
| | | | | FFLGGV | | | | | | | | |
| | | | | TLPLKD | | | | | | | | |
| | | | | ENLSKE | | | | | | | | |
| | | | | TVKW | | | | | | | | |
| 454 | NM_016546.2: | NP_057630.2: | 135 | ESHNFS | 1062 | HLA-B*57: | 12.88 | 11.30657953 | 1 | No | No | NA |
| | c.1051del | p.Leu351Trp | | GDIALL | | 01 | | | | | | |
| | | fsTer35 | | ELQHSI | | | | | | | | |
| | | | | PLGPNV | | | | | | | | |
| | | | | LPVCLP | | | | | | | | |
| | | | | DNETLY | | | | | | | | |
| | | | | RSGL | | | | | | | | |
| 455 | NM_138995.4: | NP_620482.3: | 139 | LSPVDC | 1063 | HLA-B*08: | 21.99 | 0.086419346 | 1 | No | No | NA |
| | c.3988del | pSer1330Leu | | IPEENN | | 01 | | | | | | |
| | | fsTer102 | | SAHPSF | | | | | | | | |
| | | | | FSSSSK | | | | | | | | |
| | | | | GDSFAQ | | | | | | | | |
| | | | | H | | | | | | | | |
| 456 | NM_014363.5: | NP_055178.3: | 140 | LKIEET | 1064 | HLA-A*11: | 47.3 | 12.01526875 | 1 | No | No | NA |
| | c.5151del | pLys1717Asn | | NPSLAQ | | 01 | | | | | | |
| | | fsTer8 | | DTVIIK | | | | | | | | |
| | | | | KKSCSS | | | | | | | | |
| | | | | KALNTP | | | | | | | | |
| | | | | VLSVLK | | | | | | | | |
| | | | | EAAK | | | | | | | | |
| 457 | NM_020722.1: | NP_065773.1: | 141 | AIARLD | 1065 | HLA-C*16: | 46.11 | 18.50746611 | 1 | No | No | NA |
| | c.501del | p.Lys167Asn | | NSAAKH | | 01 | | | | | | |
| | | fsTer82 | | KLAVKP | | | | | | | | |
| | | | | KKQRVS | | | | | | | | |
| | | | | KKHRRL | | | | | | | | |
| | | | | AQDPQH | | | | | | | | |
| | | | | EQGG | | | | | | | | |
| 458 | NM_004996.3: | NP_004987.2: | 146 | DAQRFM | 1066 | HLA-B*44: | 13.73 | 1.114742071 | 1 | No | No | NA |
| | c.1345dup | p.Leu449Pro | | DLATYI | | 03 | | | | | | |
| | | fsTer124 | | NMIWSA | | | | | | | | |
| | | | | PLQVIL | | | | | | | | |
| | | | | ALYLLW | | | | | | | | |
| | | | | LNLGPS | | | | | | | | |
| | | | | VLAG | | | | | | | | |
| 459 | NM_001012426.1: | NP_001012426.1: | 149 | AAVCPT | 1067 | HLA-C*16: | 21.92 | 0.640885873 | 1 | No | No | NA |
| | c.711del | p.Gln239Ser | | DLPQLW | | 01 | | | | | | |
| | | fsTer128 | | KGEGAP | | | | | | | | |
| | | | | GQPAED | | | | | | | | |
| | | | | SVKQEG | | | | | | | | |
| | | | | LDLTGT | | | | | | | | |
| | | | | AATA | | | | | | | | |
| 460 | NM_001256798.1: | NP_001243727.1: | 151 | DGLRSR | 912 | HLA-B*44: | 52.58 | 1.526850439 | 1 | Yes | No | NA |
| | c.1128dup | p.Tyr377Leu | | VKYGVK | | 03 | | | | | | |
| | | fsTer18 | | TTPESP | | | | | | | | |
| | | | | PYSSGS | | | | | | | | |
| | | | | YDSIKT | | | | | | | | |
| | | | | EVSGCP | | | | | | | | |
| | | | | EDLT | | | | | | | | |
| 461 | NM_053275.3: | NP_444505.1: | 155 | LALSVE | 1068 | HLA-C*16: | 49.59 | 2.141099432 | 1 | No | No | NA |
| | c.804_806del | p.Phe268del | | TDYTFP | | 01 | | | | | | |
| | | | | LAEKVK | | | | | | | | |
| | | | | AFLADP | | | | | | | | |
| | | | | SAFVAA | | | | | | | | |
| | | | | APVAAA | | | | | | | | |
| | | | | TTAA | | | | | | | | |
| 462 | NM_173829.3: | NP_776190.1: | 157 | QKYQKK | 1069 | HLA-C*07: | 129.42 | 8.043683467 | 1 | No | No | NA |
| | c.381del | p.Lys129Asn | | EKKKEK | | 01 | | | | | | |
| | | fsTer29 | | KSKSKK | | | | | | | | |
| | | | | GKHHKK | | | | | | | | |
| | | | | EKKKRK | | | | | | | | |
| | | | | KEKHSS | | | | | | | | |
| | | | | TPNS | | | | | | | | |
| 463 | NM_177454.3: | NP_803237.3: | 159 | TVFLTA | 1070 | HLA-C*16: | 35.14 | 0.178735949 | 1 | No | No | NA |
| | c.1113dup | pAla372Cys | | ILGGTI | | 01 | | | | | | |
| | | fsTer23 | | VIVIGF | | | | | | | | |
| | | | | FAVLLC | | | | | | | | |
| | | | | YCRDKC | | | | | | | | |
| | | | | GTPQKR | | | | | | | | |
| | | | | ERNI | | | | | | | | |
| 464 | NM_001245002.1: | NP_001231931.1: | 162 | LSAQML | 1071 | HLA-C*07: | 131.86 | 234.3696439 | 1 | No | No | NA |
| | c.1367de1 | p.Pro456Leu | | APPPPG | | 01 | | | | | | |
| | | fsTer36 | | LPRLAL | | | | | | | | |
| | | | | PPATKP | | | | | | | | |
| | | | | ATTSEG | | | | | | | | |
| | | | | GATSPT | | | | | | | | |
| | | | | SPSY | | | | | | | | |
| 465 | NM_024818.3: | NP_079094.1: | 163 | LNFGTV | 1072 | HLA-A*29: | 6.54 | 1.455171443 | 1 | No | No | NA |
| | c.876dup | p.Pro293Ser | | SFYLGY | | 02 | | | | | | |
| | | fsTer12 | | NAMQDF | | | | | | | | |
| | | | | FPTMSM | | | | | | | | |
| | | | | KPNPQC | | | | | | | | |
| | | | | DDRNCR | | | | | | | | |
| | | | | KQQE | | | | | | | | |
| 466 | NM_000213.4: | NP_000204.3: | 167 | VLVHKK | 1073 | HLA-A*11: | 127.32 | 44.63939167 | 2 | No | No | NA |
| | c.2149del | p.Leu717Cys | | KDCPPG | | 01 | | | | | | |
| | | fsTer52 | | SFWWLI | | | | | | | | |
| | | | | PLLLLL | | | | | | | | |
| | | | | LPLLAL | | | | | | | | |
| | | | | LLLLCW | | | | | | | | |
| | | | | KYCA | | | | | | | | |
| 467 | NM_006015.4: | NP_006006.3: | 168 | PNLMPS | 1074 | HLA-A*68: | 14.56 | 23.15821122 | 1 | No | No | NA |
| | c.3977del | p.Pro1326Arg | | NPDSGM | | 01 | | | | | | |
| | | fsTer155 | | YSPSRY | | | | | | | | |
| | | | | PPQQQQ | | | | | | | | |
| | | | | QQQQRH | | | | | | | | |
| | | | | DSYGNQ | | | | | | | | |
| | | | | FSTQ | | | | | | | | |
| 468 | NM_003925.2: | NP_003916.1: | 171 | ACGETL | 834 | HLA-B*44: | 100.42 | 3.950365507 | 1 | No | No | NA |
| | c.939dup | p.Glu314Arg | | SVTSEE | | 03 | | | | | | |
| | | fsTer13 | | NSLVKK | | | | | | | | |
| | | | | KERSLS | | | | | | | | |
| | | | | SGSNFC | | | | | | | | |
| | | | | SEQKTS | | | | | | | | |
| | | | | GIIN | | | | | | | | |
| 469 | NM_022470.3: | NP_071915.1: | 172 | LCNVTL | 1075 | HLA-C*16: | 27.97 | 0.567026138 | 1 | No | No | NA |
| | c.278del | p.Asn93Ile | | NSAQQA | | 01 | | | | | | |
| | | fsTer21 | | QAHYQG | | | | | | | | |
| | | | | KNHGKK | | | | | | | | |
| | | | | LRNYYA | | | | | | | | |
| | | | | ANSCPP | | | | | | | | |
| | | | | PARM | | | | | | | | |
| 470 | NM_001318252.1: | NP_001305181.1: | 176 | VQKAEA | 1076 | HLA-B*08: | 23.18 | 0.153634394 | 2 | No | No | NA |
| | c.535del | p.Leu179Cys | | LMRELD | | 01 | | | | | | |
| | | fsTer136 | | EEGSDP | | | | | | | | |
| | | | | PLPGRA | | | | | | | | |
| | | | | QRIRQV | | | | | | | | |
| | | | | LQLLS | | | | | | | | |
| 471 | NM_015446.4: | NP_056261.4: | 183 | SDLSSQ | 1077 | HLA-C*12: | 95.47 | 5.324297496 | 1 | No | No | NA |
| | c.6518del | p.Asn2173Thr | | FVISPP | | 03 | | | | | | |
| | | fsTer12 | | ALRSRQ | | | | | | | | |
| | | | | KNTSNK | | | | | | | | |
| | | | | NKLEDE | | | | | | | | |
| | | | | LKDDAQ | | | | | | | | |
| | | | | SVET | | | | | | | | |
| 472 | NM_001199380.1: | NP_0011863091: | 187 | NSPTWS | 1078 | HLA-C*16: | 29.8 | 0.809614846 | 1 | No | No | NA |
| | c.85_86del | p.Asn29Gln | | LQVFSK | | 01 | | | | | | |
| | | fsTer43 | | KKKKKK | | | | | | | | |
| | | | | KNNMAA | | | | | | | | |
| | | | | KEKLEA | | | | | | | | |
| | | | | VLNVAL | | | | | | | | |
| | | | | RVPS | | | | | | | | |
| 473 | NM_001164665.1: | NP_001158137.1: | 197 | YLESSL | 1079 | HLA-C*16: | 35.65 | 0.685593482 | 1 | No | No | NA |
| | c.2315_2316dup | p.Gly773Pro | | ISHESA | | 01 | | | | | | |
| | | fsTer9 | | VTALVP | | | | | | | | |
| | | | | PGSESF | | | | | | | | |
| | | | | DILTAG | | | | | | | | |
| | | | | IQATSP | | | | | | | | |
| | | | | LTTV | | | | | | | | |
| 474 | NM_203350.2: | NP_976225.1: | 198 | ERNRKR | 1080 | HLA-A*29: | 76.83 | 0.647951519 | 1 | No | No | NA |
| | c.901dup | p.Arg301Lys | | SRSRSS | | 02 | | | | | | |
| | | fsTer34 | | SSGDRK | | | | | | | | |
| | | | | KRRTRS | | | | | | | | |
| | | | | RSPERR | | | | | | | | |
| | | | | HRSSSG | | | | | | | | |
| | | | | SSHS | | | | | | | | |
| 475 | NM_001135919.1: | NP_001129391.1: | 202 | SGKRRF | 1081 | HLA-B*58: | 11.05 | 0.384373811 | 1 | No | No | NA |
| | c.816del | p.Phe272Leu | | LLCLLL | | 01 | | | | | | |
| | | fsTer3 | | FTVITY | | | | | | | | |
| | | | | FFVVIG | | | | | | | | |
| | | | | IAPIFI | | | | | | | | |
| | | | | LYELDS | | | | | | | | |
| | | | | PLCW | | | | | | | | |
| 476 | NM_001270.2: | NP_001261.2: | 205 | LQTRAD | 1082 | HLA-A*33: | 55.59 | 33.14096218 | 1 | No | No | NA |
| | c.3960del | p.Glu1321Lys | | YLIKLL | | 01 | | | | | | |
| | | fsTer22 | | SRDLAK | | | | | | | | |
| | | | | KEALSG | | | | | | | | |
| | | | | AGSSKR | | | | | | | | |
| | | | | RKARAK | | | | | | | | |
| | | | | KNKA | | | | | | | | |
| 477 | NM_176814.4: | NP_789784.2: | 209 | SSEIKV | 1083 | HLA-C*16: | 23.2 | 6.746594962 | 1 | No | No | NA |
| | c.1262dup | p.Ser422Phe | | KVEPAD | | 01 | | | | | | |
| | | fsTer9 | | SVESSP | | | | | | | | |
| | | | | PSITHS | | | | | | | | |
| | | | | PQNELK | | | | | | | | |
| | | | | GTNHSN | | | | | | | | |
| | | | | EKKN | | | | | | | | |
| 478 | NM_001146283.1: | NP_001139755.1: | 221 | VSPLPC | 1084 | HLA-B*08: | 56.98 | 4.060680516 | 1 | No | No | NA |
| | c.537dup | p.Ser180Leu | | CTQGHD | | 01 | | | | | | |
| | | fsTer29 | | CQHFYP | | | | | | | | |
| | | | | PSDFTV | | | | | | | | |
| | | | | STQVFR | | | | | | | | |
| | | | | DMKRSH | | | | | | | | |
| | | | | SLQK | | | | | | | | |
| 479 | NM_001146694.1: | NP_001140166.1: | 222 | HVSQAQ | 1085 | HLA-C*16: | 171.06 | 25.68193545 | 1 | No | No | NA |
| | c.3110de1 | p.Ser1037Thr | | QETYLG | | 01 | | | | | | |
| | | fsTer37 | | FWINSK | | | | | | | | |
| | | | | KSQCNI | | | | | | | | |
| | | | | FLSGTY | | | | | | | | |
| 480 | NM_024603.2: | NP_078879.2: | 227 | IWGTDV | 1086 | HLA-B*08: | 43.53 | 0.457824463 | 1 | No | No | NA |
| | c.1052del | p.Lys351Arg | | LKNRSV | | 01 | | | | | | |
| | | fsTer15 | | TGVATK | | | | | | | | |
| | | | | KKKDAV | | | | | | | | |
| | | | | PKPPLS | | | | | | | | |
| | | | | PHKLSI | | | | | | | | |
| | | | | VREC | | | | | | | | |
| 481 | NM_017763.4 | NP_060233.3: | 230 | IRQHPG | 1087 | HLA-A*68: | 40.23 | 4.044168867 | 1 | No | No | NA |
| | | p.Pro361Leu | | HAHYHL | | 01 | | | | | | |
| | | fsTer58 | | PAAYLL | | | | | | | | |
| | | | | GPSRSA | | | | | | | | |
| | | | | VARPPR | | | | | | | | |
| | | | | PGPFLP | | | | | | | | |
| | : | | | SQEP | | | | | | | | |
| | c.1082del | | | | | | | | | | | |
| 482 | NM_015208.4: | NP_056023.3: | 232 | KHMSLS | 1088 | HLA-A* | 34.52 | 38.36741167 | 1 | Yes | No | NA |
| | c.4577del | p.Asn1526Met | | YVANQE | | 02:01 | | | | | | |
| | | fsTer10 | | PGILQQ | | | | | | | | |
| | | | | KNAVQI | | | | | | | | |
| | | | | ISSALD | | | | | | | | |
| | | | | TDNEST | | | | | | | | |
| | | | | KDTE | | | | | | | | |
| 483 | NM_017654.3: | NP_060124.2: | 233 | QIMLDM | 1089 | HLA-B* | 3.78 | 1.136281689 | 1 | No | No | NA |
| | c.3073del | p.Ser1025Val | | LTENLF | | 08:01 | | | | | | |
| | | fsTer79 | | FDTGMG | | | | | | | | |
| | | | | KSKFLQ | | | | | | | | |
| | | | | DMHTLL | | | | | | | | |
| | | | | LTRHRD | | | | | | | | |
| | | | | EHEG | | | | | | | | |
| 484 | NM_003489.3: | NP_003480.2: | 237 | KVTLLQ | 1090 | HLA-B* | 189.19 | 389.2543615 | 1 | No | No | NA |
| | c.1547del | p.Asn516Thr | | LLLGHK | | 08:01 | | | | | | |
| | | fsTer11 | | NEENVE | | | | | | | | |
| | | | | KNTSPQ | | | | | | | | |
| | | | | GVHNDV | | | | | | | | |
| | | | | SKFNTQ | | | | | | | | |
| | | | | NYAR | | | | | | | | |
| 485 | NM_015412.3: | NP_056227.2: | 249 | SFTQLS | 1091 | HLA-A* | 31.58 | 12.3569003 | 1 | No | No | NA |
| | c.1155del | pLys385Asn | | EEIQMA | | 11:01 | | | | | | |
| | | fsTer33 | | VVWCRS | | | | | | | | |
| | | | | KKLKAQ | | | | | | | | |
| | | | | AIFLGN | | | | | | | | |
| | | | | KLLKSN | | | | | | | | |
| | | | | RLKH | | | | | | | | |
| 486 | NM_053043.2: | NP_444271.2: | 254 | PPRQPF | 1092 | HLA-C* | 90.96 | 1.676333054 | 1 | No | No | NA |
| | c.1723_1724dup | p.Gln575His | | LPGPGQ | | 16:01 | | | | | | |
| | | fsTer76 | | PFLPTH | | | | | | | | |
| | | | | TQPNLQ | | | | | | | | |
| | | | | GPLHPP | | | | | | | | |
| | | | | LPPPHQ | | | | | | | | |
| | | | | PQPQ | | | | | | | | |
| 487 | NM_005788.3: | NP_005779.1: | 261 | TISLVA | 1093 | HLA-A* | 29.48 | 22.74415789 | 1 | No | No | NA |
| | c.1147del | p.Trp383Gly | | VSDVNK | | 68:01 | | | | | | |
| | | fsTer12 | | HADRIA | | | | | | | | |
| | | | | FWDDVY | | | | | | | | |
| | | | | GFKMSC | | | | | | | | |
| | | | | MKKAVI | | | | | | | | |
| | | | | PEAV | | | | | | | | |
| 488 | NM_001330315.1: | NP_001317244.1: | 271 | LKTVGK | 1094 | HLA-C* | 108.27 | 4.256775848 | 1 | No | No | NA |
| | c.742del | p.Cys248Ala | | LTATQV | | 12:03 | | | | | | |
| | | fsTer22 | | AKISFF | | | | | | | | |
| | | | | FCFVWF | | | | | | | | |
| | | | | LANLSY | | | | | | | | |
| | | | | QEALSD | | | | | | | | |
| | | | | TQVA | | | | | | | | |
| 489 | NM_014672.3: | NP_055487.2: | 275 | YLRKDE | 1095 | HLA-A* | 65.82 | 1.085190394 | 1 | Yes | No | NA |
| | c.257dup | pLeu86Phe | | GSNKQV | | 29:02 | | | | | | |
| | | fsTer6 | | YSVPHF | | | | | | | | |
| | | | | FLAGAA | | | | | | | | |
| | | | | KERSQM | | | | | | | | |
| | | | | NSQTED | | | | | | | | |
| | | | | HALA | | | | | | | | |
| 490 | NM_001085471.1: | NP_001078940.1: | 285 | GESVLR | 1096 | HLA-A* | 11.18 | 0.376201755 | 1 | No | No | NA |
| | c.287del | p.Pro96His | | SVSPVQ | | 68:01 | | | | | | |
| | | fsTer42 | | DLDDDT | | | | | | | | |
| | | | | PPSPAH | | | | | | | | |
| | | | | SDMPYD | | | | | | | | |
| | | | | ARQNPN | | | | | | | | |
| | | | | CKPP | | | | | | | | |
| 491 | NM_194318.3: | NP_919299.3: | 288 | VIQSQS | 1097 | HLA-B* | 85.55 | 0.237459849 | 1 | No | No | NA |
| | c.238dup | p.Ser80Lys | | NSFHAK | | 08:01 | | | | | | |
| | | fsTer24 | | RAEQLK | | | | | | | | |
| | | | | KSILKQ | | | | | | | | |
| | | | | AADLTQ | | | | | | | | |
| | | | | ELPSVL | | | | | | | | |
| | | | | LLHQ | | | | | | | | |
| 492 | NM_001167856.2: | NP_001161328.1: | 289 | VSNDLK | 1098 | HLA-B* | 216.03 | 5.827329507 | 1 | No | No | NA |
| | c.1109dup | p.Asn370Lys | | YDAERD | | 08:01 | | | | | | |
| | | fsTer9 | | LRDIGA | | | | | | | | |
| | | | | KNILVH | | | | | | | | |
| | | | | SLNKFK | | | | | | | | |
| | | | | YGKISS | | | | | | | | |
| | | | | KHNG | | | | | | | | |
| 493 | NM_014727.1: | NP_055542.1: | 291 | ARSSRV | 1099 | HLA-B* | 3.4 | 9.786184901 | 1 | No | No | NA |
| | c.1656del | p.Lys553Asn | | IKTPRR | | 07:02 | | | | | | |
| | | fsTer52 | | FMDEDP | | | | | | | | |
| | | | | PKPPKV | | | | | | | | |
| | | | | EVSPVL | | | | | | | | |
| | | | | RPPITT | | | | | | | | |
| | | | | SPPV | | | | | | | | |
| 494 | NM_152243.2: | NP_689449.1: | 298 | PRSFLA | 1100 | HLA-A* | 15.57 | 1.831096417 | 1 | No | No | NA |
| | c.289del | p.Arg97Gly | | KKLQLV | | 11:01 | | | | | | |
| | | fsTer115 | | RRVGAP | | | | | | | | |
| | | | | PRRMAS | | | | | | | | |
| | | | | PPAPSP | | | | | | | | |
| | | | | APPAIS | | | | | | | | |
| | | | | PIIK | | | | | | | | |
| 495 | NM_001135146.1: | NP_0011286181: | 312 | NGHIHF | 1101 | HLA-C* | 74.17 | 0.711403012 | 1 | No | No | NA |
| | c.852dup | p.Glu285Arg | | DNVSVV | | 16:01 | | | | | | |
| | | fsTer59 | | SLQDGK | | | | | | | | |
| | | | | KEPSSC | | | | | | | | |
| | | | | TCLKGP | | | | | | | | |
| | | | | KLSEIG | | | | | | | | |
| | | | | TIAW | | | | | | | | |
| 496 | NM_006585.2: | NP_006576.2: | 315 | WAIKLA | 1102 | HLA-A* | 48.59 | 0.69925027 | 1 | No | No | NA |
| | c.1575_1577del | p.Ile525del | | TNAAVT | | 11:01 | | | | | | |
| | | | | VLRVDQ | | | | | | | | |
| | | | | IIMAKP | | | | | | | | |
| | | | | AGGPKP | | | | | | | | |
| | | | | PSGKKD | | | | | | | | |
| | | | | WDDD | | | | | | | | |
| 497 | NM_001500.3: | NP_001491.1: | 328 | ARCPSA | 1103 | HLA-A* | 6.67 | 0.128013097 | 1 | No | No | NA |
| | c.69_73dup | p.Val25Gly | | RGSGDG | | 68:01 | | | | | | |
| | | fsTer58 | | EMGKPR | | | | | | | | |
| | | | | NVALIT | | | | | | | | |
| | | | | GITGQD | | | | | | | | |
| | | | | GSYLAE | | | | | | | | |
| | | | | FLLE | | | | | | | | |
| 498 | NM_007214.4: | NP_009145.1: | 330 | KGGWQQ | 1104 | HLA-A* | 29.25 | 0.878237466 | 1 | No | No | NA |
| | c.1585_1586del | p.Lys529Glu | | KSKGPK | | 11:01 | | | | | | |
| | | fsTer30 | | KTAKSK | | | | | | | | |
| | | | | KKKPLK | | | | | | | | |
| | | | | KKPTPV | | | | | | | | |
| | | | | LLPQSK | | | | | | | | |
| | | | | QQKQ | | | | | | | | |
| 499 | NM_001323897.1: | NP_0013108261: | 336 | FLLGRW | 1105 | HLA-B* | 46.69 | 0.42503908 | 1 | No | No | NA |
| | c.1234dup | p.Thr412Asn | | CYQVSH | | 08:01 | | | | | | |
| | | fsTer7 | | LSWLEK | | | | | | | | |
| | | | | KTATAL | | | | | | | | |
| | | | | LESPLS | | | | | | | | |
| | | | | ATVEDA | | | | | | | | |
| | | | | LQSF | | | | | | | | |
| 500 | NM_144682.5: | NP_653283.3: | 342 | DSLKNV | 1106 | HLA-C*16: | 160.79 | 4.067830853 | 1 | No | No | NA |
| | c.856dup | pCys286Leu | | IARAIS | | 01 | | | | | | |
| | | fsTer30 | | KLPIVH | | | | | | | | |
| | | | | FCSSKP | | | | | | | | |
| | | | | RVEYST | | | | | | | | |
| | | | | KIVEVF | | | | | | | | |
| | | | | CGKE | | | | | | | | |
| 501 | NM_019589.2: | NP_062535.2: | 346 | LPTMPP | 1107 | HLA-A*02: | 45.02 | 74.31176528 | 1 | No | No | NA |
| | c.1646del | p.Pro549Leu | | PVLPPS | | 01 | | | | | | |
| | | fsTer98 | | LPPPVM | | | | | | | | |
| | | | | PPALPA | | | | | | | | |
| | | | | TVPPPG | | | | | | | | |
| | | | | MPPPVM | | | | | | | | |
| | | | | PPSL | | | | | | | | |
| 502 | NM_014718.3: | NP_055533.2: | 347 | DSEVAD | 1108 | HLA-B*07: | 42.84 | 45.25526815 | 1 | No | No | NA |
| | c.2858del | pPro953His | | SPSSDE | | 02 | | | | | | |
| | | fsTer106 | | RRIIET | | | | | | | | |
| | | | | PPHRY | | | | | | | | |

Supplementary Table 9. List of the Top 100 most recurrent predicted MHC-I neoAgs, with immunogenic score,
obtained from the computational methods in the validation set.

| | | | | | | | Reference | | | Number | |
|---|---|---|---|---|---|---|---|---|---|---|---|
| Mutant | SEQ | | | | | Micro- | MS | | Altered MS | deleted | |
| Epitope | ID | Gene | Chromo- | | | satellite | Lengths | Variant | Length | nucleo- | Peptide |
| Sequence | NO | Name | some | Start | Stop | motif | (repeats) | Type | (repeats) | tides | Length |
| MSVCFFFFCY | 503 | CNOT1 | chr16 | 58577316 | 58577328 | A | 13 | FS | 12 | −1 | 10 |
| VMSDTTYKIY | 504 | TTK | chr6 | 80751896 | 80751897 | A | 9 | FS | 8 | −1 | 10 |
| QAHPQVPAL | 505 | ZBTB20 | chr3 | 114058002 | 114058003 | G | 7 | FS | 6 | −1 | 9 |
| FPITPPVWHIL | 506 | RNF43 | chr17 | 56435160 | 56435161 | G | 7 | FS | 6 | −1 | 11 |
| FPITPPVWHIL | 506 | RNF43 | chr17 | 56435160 | 56435161 | G | 7 | FS | 8 | −1 | 11 |
| VVHKKRGL | 507 | ACVR2A | chr2 | 148683685 | 148683686 | A | 8 | FS | 7 | −1 | 8 |
| RSAFPSRSL | 126 | MARC | chr6 | 114181210 | 114181220 | A | 11 | FS | 10 | −1 | 9 |
| | | KS | | | | | | | | | |
| SRYPNICWF | 508 | CNOT1 | chr16 | 58577316 | 58577328 | A | 13 | FS | 11 | −1 | 9 |
| MTTISRATW | 509 | XYLT2 | chr17 | 48433967 | 48433973 | C | 7 | FS | 6 | −1 | 9 |
| SSWATCWPR | 510 | MXRA8 | chr1 | 1290109 | 1290110 | C | 7 | FS | 6 | −1 | 9 |
| RVRAHPGLPR | 511 | TSC22 | chr7 | 100075308 | 100075309 | G | 5 | FS | 4 | −1 | 10 |
| | | D4 | | | | | | | | | |
| STAHIPPLHLR | 512 | TCF7 | chr5 | 133473764 | 133473765 | C | 7 | FS | 6 | −1 | 11 |
| LMLLRLNL | 409 | SEC31 | chr4 | 83785564 | 83785565 | A | 9 | FS | 8 | −1 | 8 |
| | | A | | | | | | | | | |
| LMLLRLNL | 409 | SEC31 | chr4 | 83785564 | 83785565 | T | 9 | FS | 8 | −1 | 8 |
| | | A | | | | | | | | | |
| HSYHLLQAY | 422 | SETD5 | chr3 | 9486784 | 9486785 | A | 6 | FS | 5 | −1 | 9 |
| KRATFLLAL | 424 | MSH3 | chr5 | 79970914 | 79970915 | A | 8 | FS | 7 | −1 | 9 |
| FSDSEGEGL | 433 | REST | chr4 | 57777065 | 57777068 | AAG | 4 | FS | 3 | −2 | 9 |
| YADQWTVL | 435 | STAT5 | chr1 | 40461419 | 40461420 | G | 5 | FS | 4 | −1 | 8 |
| | | A | | | | | | | | | |
| KSFTKNHSSK | 450 | ZNF585B | chr19 | 37676332 | 37676333 | A | 6 | FS | 5 | −1 | 10 |
| TEVTWWALRK | 466 | ITGB4 | chr17 | 73733649 | 73733650 | C | 5 | FS | 4 | −1 | 10 |
| CLRPHRVQL | 470 | C7orf50 | chr7 | 1037310 | 1037311 | C | 7 | FS | 6 | −1 | 9 |
| RLWNKTRCR | 513 | SH3B | chr1 | 26607374 | 26607375 | C | 4 | FS | 3 | −1 | 9 |
| | | GRL3 | | | | | | | | | |
| SSFLVSSSI | 514 | SPECC1L | chr22 | 24718455 | 24718456 | NA | NA | FS | NA | −1 | 9 |
| TFMPPPGHPPR | 515 | TMEM79 | chr1 | 156255497 | 156255497 | C | 6 | FS | 7 | 1 | 11 |
| KTNATFSLV | 516 | KDM4 | chr9 | 7170005 | 7170006 | A | 7 | FS | ins G | −1 | 9 |
| | | C | | | | | | | | | |
| AQTRDRWRK | 517 | MGME1 | chr20 | 17950704 | 17950705 | G | 4 | FS | 3 | −1 | 9 |
| STEVEETQEK | 518 | ATP6V1G1 | chr9 | 117359886 | 117359889 | NA | NA | FS | NA | −2 | 10 |
| TFKKKTYTC | 519 | SEC63 | chr6 | 108214773 | 108214773 | A | 9 | FS | 10 | 1 | 9 |
| LPPPGCGSW | 520 | TEAD3 | chr6 | 35446236 | 35446237 | C | 4 | FS | 3 | −1 | 9 |
| HARGSSLITM | 521 | ITGA6 | chr2 | 173330355 | 173330356 | G | 5 | FS | 4 | −1 | 10 |
| MNKDLLRVL | 522 | PPP4R3B | chr2 | 55800797 | 55800800 | AGA | 2 | FS | 1 | −2 | 9 |
| VAARWHGTL | 523 | GATA3 | chr10 | 8100728 | 8100734 | C | 7 | FS | 8 | 1 | 9 |
| VAISFKTVF | 524 | AVPR1A | chr12 | 63541343 | 63541348 | T | 6 | FS | 5 | −1 | 9 |
| QTHWRLYPK | 525 | CCDC168 | chr13 | 103381996 | 103382004 | T | 9 | FS | 8 | −1 | 9 |
| LTMAPSCLR | 526 | TGM6 | chr20 | 2384126 | 2384131 | C | 6 | FS | 5 | .1 | 9 |
| MPQACDGLTW | 527 | PRELP | chr1 | 203452549 | 203452554 | C | 6 | FS | 5 | −1 | 10 |
| SAWTGSVSV | 528 | TNR | chr1 | 175372615 | 175372620 | G | 6 | FS | 5 | −1 | 9 |
| YPAASAPCW | 529 | ZDHH | chr22 | 20130522 | 20130527 | C | 6 | FS | 5 | −1 | 9 |
| | | C8 | | | | | | | | | |
| LPSSTLWTF | 530 | ENTP | chr9 | 139945517 | 139945522 | C | 6 | FS | 5 | −1 | 9 |
| | | D2 | | | | | | | | | |
| LPCNVTFLM | 531 | PKHD1 | chr6 | 51890015 | 51890021 | T | 7 | FS | 6 | −1 | 9 |
| YPTSVHYQTPW | 532 | TULP4 | chr6 | 158923337 | 158923342 | C | 6 | FS | 5 | −1 | 11 |
| NSLPPAALR | 533 | CARM | chr16 | 67682131 | 67682136 | C | 6 | FS | 7 | 1 | 9 |
| | | IL2 | | | | | | | | | |
| VAHDPPQSL | 534 | AMDH | chr16 | 2578619 | 2578625 | C | 7 | FS | 8 | 1 | 9 |
| | | D2 | | | | | | | | | |
| SASGSPWPM | 535 | ARAF | chrX | 47426415 | 47426420 | C | 6 | FS | 5 | −1 | 9 |
| LLFPASGEM | 536 | COL4 | chr13 | 111156324 | 111156330 | C | 7 | FS | 6 | −1 | 9 |
| | | A2 | | | | | | | | | |
| APAPPTRCVW | 537 | MVK | chr12 | 110019240 | 110019245 | C | 6 | FS | 5 | −1 | 10 |
| SPQAPLRLW | 538 | BCOR | chrX | 129190011 | 129190017 | C | 7 | FS | 6 | −1 | 9 |
| | | L1 | | | | | | | | | |
| SSHPRPLPA | 539 | BCL9L | chr11 | 118773097 | 118773098 | C | 15 | FS | 14 | −1 | 9 |
| SSARGWAPCK | 540 | THEM | chr1 | 28208611 | 28208612 | C | 5 | FS | 4 | −1 | 10 |
| | | IS2 | | | | | | | | | |
| IAKKMTSTL | 541 | TDRD15 | chr2 | 21362231 | 21362232 | A | 7 | FS | 6 | −1 | 9 |
| RAMPSSGAA | 542 | SPTBN5 | chr15 | 42145897 | 42145902 | G | 6 | FS | 5 | −1 | 9 |
| RTFCLTARR | 543 | PRX | chr19 | 40900434 | 40900436 | TC | 2 | FS | 1 | −1 | 9 |
| WACPHPRSL | 544 | TBC1 | chrX | 48419061 | 48419062 | T | 4 | FS | 3 | −1 | 9 |
| | | D25 | | | | | | | | | |
| FGPSLVRGLW | 545 | TMEM201 | chr1 | 9673074 | 9673075 | NA | NA | FS | NA | −1 | 10 |
| SASSLAAAL | 546 | BARH | chr9 | 135464864 | 135464865 | C | 7 | FS | 6 | −1 | 9 |
| | | L1 | | | | | | | | | |
| SMRSTRWAR | 547 | SPG7 | chr16 | 89598370 | 89598371 | C | 7 | FS | 6 | −1 | 9 |
| ATLSAAFARR | 548 | GZF1 | chr20 | 23345851 | 23345852 | A | 5 | FS | 4 | −1 | 10 |
| SPFPPSSLHW | 549 | ZFR2 | chr19 | 3831692 | 3831693 | C | 6 | FS | 5 | −1 | 10 |
| TAAACPTPV | 550 | JAG2 | chr14 | 105614480 | 105614481 | C | 7 | FS | 6 | −1 | 9 |
| EAAPPSTSM | 551 | CSF1R | chr5 | 149460475 | 149460476 | C | 6 | FS | 5 | −1 | 9 |
| TTQAPLRAAR | 552 | SCN10 | chr3 | 38766674 | 38766675 | NA | NA | FS | NA | −1 | 10 |
| | | A | | | | | | | | | |
| RPFRTRMTW | 553 | MAGE | chrX | 75004753 | 75004754 | C | 6 | FS | 5 | −1 | 9 |
| | | E2 | | | | | | | | | |
| SPSMGAMRW | 554 | ABCD1 | chrX | 152991548 | 152991549 | G | 5 | FS | 4 | −1 | 9 |
| CQPALLEQLF | 555 | BEST4 | chr1 | 45250035 | 45250036 | C | 4 | FS | 3 | −1 | 10 |
| VSLPPPPLQR | 556 | KCNH4 | chr17 | 40328259 | 40328265 | G | 7 | FS | 8 | 1 | 10 |
| ATQEVQMRSR | 557 | IFFO2 | chr1 | 19235144 | 19235145 | C | 5 | FS | 4 | −1 | 10 |
| SIFPFQKTL | 558 | PCDH9 | chr13 | 67802152 | 67802153 | C | 5 | FS | 4 | −1 | 9 |
| SPHLPNPTW | 559 | YLPM1 | chr14 | 75230470 | 75230471 | G | 6 | FS | 5 | −1 | 9 |
| SVSWSIPRR | 560 | EPHA2 | chr1 | 16462198 | 16462199 | C | 6 | FS | 5 | −1 | 9 |
| LDTMSCPPWK | 561 | NECTI | chr19 | 45389229 | 45389230 | A | 4 | FS | 3 | −1 | 10 |
| | | N2 | | | | | | | | | |
| RAGGMAPAK | 562 | CEP250 | chr20 | 34061365 | 34061367 | TC | 3 | FS | 2 | −1 | 9 |
| MASTVTEVLR | 563 | COL9 | chr6 | 70991120 | 70991121 | C | 6 | FS | 5 | −1 | 10 |
| | | A1 | | | | | | | | | |
| TLQPASGSK | 564 | PYGO2 | chr1 | 154932027 | 154932027 | C | 7 | FS | 8 | 1 | 9 |
| RSRAPRTATR | 565 | TBX2 | chr17 | 59482061 | 59482066 | C | 6 | FS | 5 | −1 | 10 |
| VAPTPQRPL | 566 | KMT2 | chr19 | 36210764 | 36210770 | C | 7 | FS | 6 | −1 | 9 |
| | | B | | | | | | | | | |
| HPLVATQAL | 567 | OBSC | chr1 | 228559449 | 228559450 | G | 7 | FS | 6 | −1 | 9 |
| | | N | | | | | | | | | |
| QVWTAATLR | 568 | DOCK3 | chr3 | 51417603 | 51417604 | C | 7 | FS | 6 | −1 | 9 |
| LSASASTSL | 569 | ADGR | chr2 | 26534412 | 26534413 | G | 7 | FS | 6 | −1 | 9 |
| | | F3 | | | | | | | | | |
| CAAWSSTRPW | 570 | CUED | chr17 | 55962834 | 55962835 | C | 6 | FS | 5 | −1 | 10 |
| | | C1 | | | | | | | | | |
| SAAAPRHSR | 571 | GJA3 | chr13 | 20716371 | 20716372 | C | 5 | FS | 4 | −1 | 9 |
| FMQNIRIPI | 572 | GDF11 | chr12 | 56143493 | 56143494 | A | 5 | FS | 4 | −1 | 9 |
| RKSHRFASGK | 573 | PRR14 | chr22 | 32099548 | 32099549 | C | 6 | FS | 5 | −1 | 10 |
| | | L | | | | | | | | | |
| STRPWTTSR | 574 | CUED | chr17 | 55962881 | 55962882 | G | 5 | FS | 4 | −1 | 9 |
| | | C1 | | | | | | | | | |
| TAAMGPHTY | 575 | HOXA3 | chr7 | 27147845 | 27147846 | C | 5 | FS | 4 | −1 | 9 |
| MTVKNLIQF | 576 | PSME4 | chr2 | 54093344 | 54093346 | A | 8 | FS | 6 | −1 | 9 |
| LPQHAPHTLLY | 577 | PPM1 | chr12 | 63195699 | 63195699 | G | 5 | FS | 6 | 1 | 11 |
| | | H | | | | | | | | | |
| SMATVARAPR | 578 | TMEM143 | chr19 | 48866745 | 48866750 | C | 6 | FS | 7 | 1 | 10 |
| CLGLWCSPR | 579 | TBC1 | chr11 | 67176564 | 67176565 | C | 7 | FS | 6 | −1 | 9 |
| | | D10C | | | | | | | | | |
| QFFEIKSR | 580 | VASH1 | chr14 | 77237566 | 77237569 | AGA | 2 | FS | 1 | −2 | 8 |
| WRPSVGLFSM | 581 | TFAP2 | chr6 | 50683292 | 50683293 | G | 4 | FS | 3 | −1 | 10 |
| | | D | | | | | | | | | |
| HGQPVLSHR | 582 | AHDC1 | chr1 | 27878527 | 27878528 | C | 14 | FS | 13 | −1 | 9 |
| APSWSSRRW | 583 | LARP6 | chr15 | 71125203 | 71125204 | T | 5 | FS | 4 | −1 | 9 |
| FSVMIPPM | 584 | PXDN | chr2 | 1652959 | 1652960 | C | 7 | FS | 6 | −1 | 8 |
| SPWTSRRGPPW | 585 | TMEM132D | chr12 | 130184704 | 130184705 | C | 7 | FS | 6 | −1 | 11 |
| ALRSPFLQGR | 586 | MEFV | chr16 | 3304412 | 3304417 | G | 6 | FS | 5 | −1 | 10 |
| KMERFWKLIR | 587 | EML6 | chr2 | 55122479 | 55122480 | A | 6 | FS | 5 | −1 | 10 |
| MNMKMKTF MK | 588 | CD79A | chr19 | 42383609 | 42383610 | C | 6 | FS | 5 | −1 | 10 |
| KGATYTPRHPR | 589 | PLEK | chr1 | 150131249 | 150131254 | C | 6 | FS | 5 | −1 | 11 |
| | | HO1 | | | | | | | | | |
| SSPTHVRAA | 590 | FIZ1 | chr19 | 56109216 | 56109217 | C | 4 | FS | 3 | −1 | 9 |
| KMWEPPMVL | 591 | ZHX2 | chr8 | 123965544 | 123965545 | A | 6 | FS | 5 | −1 | 9 |

| | | | | | | | | | | Pre- | | |
|---|---|---|---|---|---|---|---|---|---|---|---|---|
| | | | | | | | Bind- | | | dicted | | |
| | | | | | | | ing | | | in | | |
| | | | | Wild- | | | Af- | Tumor | | the | Eli- | Eli- |
| SEQ | | | Immuno- | type | SEQ | | fin- | Abun- | Sample | Dis- | spot | spot |
| ID | | | genicity | se- | ID | HLA | ity | dance | Recur- | covery | test- | reac- |
| NO | HGVSc | HGVSp | Score | quence | NO | Allele | (nM) | (TPM) | rence | Set | ed | tive |
| 503 | NM_206999.2: | NP_996882.1: | 1863 | ILDCNS | 1109 | HLA- | 35.1 | 0 | 5 | No | No | NA |
| | c.4628del | p.Leu1544Cys | | VRQSIM | | A*29: | | | | | | |
| | | fsTer11 | | SVCFFF | | 02 | | | | | | |
| | | | | FLLYSQ | | | | | | | | |
| | | | | HDV | | | | | | | | |
| 504 | NM_003318.4: | NP_003309.2: | 5 | HYSGGE | 1017 | HLA- | 18.66 | 75.69082705 | 3 | No | No | NA |
| | c.2560del | p.Arg854Gly | | SHNSSS | | A*29: | | | | | | |
| | | fsTer39 | | SKTFEK | | 02 | | | | | | |
| | | | | KRGKK | | | | | | | | |
| 505 | NM_001164342.2: | NP_001157814.1: | 67 | HKTLLE | 951 | HLA- | 33.03 | 15.82316283 | 3 | Yes | No | NA |
| | c.2075de1 | p.Pro692Leu | | RHVALH | | C*16: | | | | | | |
| | | fsTer43 | | SASNGT | | 01 | | | | | | |
| | | | | PPAGTP | | | | | | | | |
| | | | | PGARAG | | | | | | | | |
| | | | | PPGVVA | | | | | | | | |
| | | | | CTEG | | | | | | | | |
| 506 | NM_017763.4: | NP_060233.3: | 570 | FNLQKS | 784 | HLA- | 408.69 | 105.9103245 | 3 | Yes | No | NA |
| | c.1976del | p.Gly659Val | | SLSARH | | B*35: | | | | | | |
| | | fsTer41 | | PQRKRR | | 03 | | | | | | |
| | | | | GGPSEP | | | | | | | | |
| | | | | TPGSRP | | | | | | | | |
| | | | | QDATVH | | | | | | | | |
| | | | | PACQ | | | | | | | | |
| 506 | NM_017763.4: | NP_060233.3: | 570 | FNLQKS | 784 | HLA- | 408.69 | 105.9103245 | 3 | Yes | No | NA |
| | c.1976del | pGly659Val | | SLSARH | | B*35: | | | | | | |
| | | fsTer41 | | PQRKRR | | 03 | | | | | | |
| | | | | GGPSEP | | | | | | | | |
| | | | | TPGSRP | | | | | | | | |
| | | | | QDATVH | | | | | | | | |
| | | | | PACQ | | | | | | | | |
| 507 | NM_001278579.1: | NP_001265508.1: | 746 | EIGQHP | 889 | HLA- | 641.47 | 82.22872638 | 3 | Yes | Yes | No |
| | c.1310de1 | p.Lys437Arg | | SLEDMQ | | B*08: | | | | | | |
| | | fsTer5 | | EVVVHK | | 01 | | | | | | |
| | | | | KKRPVL | | | | | | | | |
| | | | | RDYWQK | | | | | | | | |
| | | | | HAGMAM | | | | | | | | |
| | | | | LCET | | | | | | | | |
| 126 | NM_002356.5: | NP_002347.5: | 1523 | PKAEDG | 888 | HLA- | 19.82 | 0 | 3 | Yes | Yes | No |
| | c.464del | p.Lys155Arg | | ATPSPS | | C*16: | | | | | | |
| | | fsTer12 | | NETPKK | | 01 | | | | | | |
| | | | | KKKRFS | | | | | | | | |
| | | | | FKKSFK | | | | | | | | |
| | | | | LSGFSF | | | | | | | | |
| | | | | KKNK | | | | | | | | |
| 508 | NM_206999.2: | NP_996882.1: | 1596 | IILDCN | 1110 | HLA- | 78.84 | 0 | 3 | No | No | NA |
| | c.4627_4628del | pPhe1543Ser | | SVRQSI | | C*07: | | | | | | |
| | | fsTer22 | | MSVCFF | | 01 | | | | | | |
| | | | | FFLLYS | | | | | | | | |
| | | | | QHDV | | | | | | | | |
| 509 | NM_022167.2: | NP_071450.2: | 1677 | VNQEVL | 859 | HLA- | 6.73 | 0 | 3 | No | No | NA |
| | c.1584del | p.Gly529Ala | | EILDFH | | B*57: | | | | | | |
| | | fsTer78 | | LYGSYP | | 01 | | | | | | |
| | | | | PGTPAL | | | | | | | | |
| | | | | KAYWEN | | | | | | | | |
| | | | | TYDAAD | | | | | | | | |
| | | | | GPSG | | | | | | | | |
| 510 | NM_001282585.1: | NP_001269514.1: | 2122 | HERRVF | 1111 | HLA- | 12.35 | 0 | 3 | Yes | No | NA |
| | c.901del | p.Arg301Gly | | HLTVAE | | A*33: | | | | | | |
| | | fsTer107 | | PHAEPP | | 01 | | | | | | |
| | | | | PRGSPG | | | | | | | | |
| | | | | NGSSHS | | | | | | | | |
| | | | | GAPGPD | | | | | | | | |
| | | | | PTLA | | | | | | | | |
| 511 | NM_030935.3: | NP_112197.1: | 3401 | DLEPHS | 1112 | HLA- | 76.76 | 0 | 3 | No | No | NA |
| | c.353del | p.Gly118Ala | | FGGLLE | | A*11: | | | | | | |
| | | fsTer99 | | GIRGAS | | 01 | | | | | | |
| | | | | GGAGGR | | | | | | | | |
| | | | | SLDSRL | | | | | | | | |
| | | | | ELASLG | | | | | | | | |
| | | | | LGAP | | | | | | | | |
| 512 | NM_001346425.1: | NP_001333354.1: | 4114 | GAGQHP | 1113 | HLA- | 125.65 | 0 | 3 | Yes | No | NA |
| | c.463del | p.His155Thr | | QPQPPL | | A*11: | | | | | | |
| | | fsTer44 | | HKANQP | | 01 | | | | | | |
| | | | | PHGVPQ | | | | | | | | |
| | | | | LSLYEH | | | | | | | | |
| | | | | FNSPHP | | | | | | | | |
| | | | | TPAP | | | | | | | | |
| 409 | NM_001318120.1: | NP_001305049.1: | 7 | DQLQQA | 789 | HLA- | 55.29 | 113.4902951 | 2 | Yes | No | NA |
| | c.1384de1 | p.Ile462Leu | | VQSQGF | | B*08: | | | | | | |
| | | fsTer16 | | INYCQK | | 01 | | | | | | |
| | | | | KIDASQ | | | | | | | | |
| | | | | TEFEKN | | | | | | | | |
| | | | | VWSFLK | | | | | | | | |
| | | | | VNFE | | | | | | | | |
| 409 | NM_001318120.1: | NP_001305049.1: | 7 | DQLQQA | 789 | HLA- | 55.29 | 113.4902951 | 2 | Yes | No | NA |
| | c.1384de1 | p.Ile462Leu | | VQSQGF | | B*08: | | | | | | |
| | | fsTer16 | | INYCQK | | 01 | | | | | | |
| | | | | KIDASQ | | | | | | | | |
| | | | | TEFEKN | | | | | | | | |
| | | | | VWSFLK | | | | | | | | |
| | | | | VNFE | | | | | | | | |
| 422 | NM_001080517.2: | NP_001073986.1: | 42 | NYKVDC | 1032 | HLA- | 11.33 | 117.8533251 | 2 | No | No | NA |
| | c.1246de1 | p.Arg416Gly | | ACHKGN | | C*12: | | | | | | |
| | | fsTer34 | | RNCPIQ | | 03 | | | | | | |
| | | | | KRNPNA | | | | | | | | |
| | | | | TELPLL | | | | | | | | |
| | | | | PPPPSL | | | | | | | | |
| | | | | PTIG | | | | | | | | |
| 424 | NM_002439.4: | NP_002430.3: | 44 | STSYLL | 830 | HLA- | 54.4 | 53.93262102 | 2 | Yes | No | NA |
| | c.1148del | p.Lys383Arg | | CISENK | | C*07: | | | | | | |
| | | fsTer32 | | ENVRDK | | 01 | | | | | | |
| | | | | KKGNIF | | | | | | | | |
| | | | | IGIVGV | | | | | | | | |
| | | | | QPATGE | | | | | | | | |
| | | | | VVFD | | | | | | | | |
| 433 | NM_005612.4: | NP_005603.3: | 75 | ERQMAE | 1042 | HLA- | 27.97 | 54.50562555 | 2 | No | No | NA |
| | c.266_268del | p.Glu89del | | LMPVGD | | C*08: | | | | | | |
| | | | | NNFSDS | | 02 | | | | | | |
| | | | | EEGEGL | | | | | | | | |
| | | | | EESADI | | | | | | | | |
| | | | | KGEPHG | | | | | | | | |
| | | | | LENM | | | | | | | | |
| 435 | NM_001288718.1: | NP_001275647.1: | 77 | KPQIKQ | 1044 | HLA- | 16.97 | 5.537065595 | 2 | No | No | NA |
| | c.2144de1 | p.Gly715Ala | | VVPEFV | | C*08: | | | | | | |
| | | fsTer101 | | NASADA | | 02 | | | | | | |
| | | | | GGSSAT | | | | | | | | |
| | | | | YMDQAP | | | | | | | | |
| | | | | SPAVCP | | | | | | | | |
| | | | | QAPY | | | | | | | | |
| 450 | NM_152279.3: | NP_689492.3: | 125 | IHTGEK | 1059 | HLA- | 72.36 | 26.87688773 | 2 | No | No | NA |
| | c.2106del | p.Lys702Asn | | PYECSD | | A*11: | | | | | | |
| | | fsTer32 | | CGKSFT | | 01 | | | | | | |
| | | | | KKSQLQ | | | | | | | | |
| | | | | VHQRIH | | | | | | | | |
| | | | | TGEKPY | | | | | | | | |
| | | | | VCAE | | | | | | | | |
| 466 | NM_000213.4: | NP_000204.3: | 167 | VLVHKK | 1073 | HLA- | 127.32 | 44.63939167 | 2 | No | No | NA |
| | c.2149del | p.Leu717Cys | | KDCPPG | | A*11: | | | | | | |
| | | fsTer52 | | SFWWLI | | 01 | | | | | | |
| | | | | PLLLLL | | | | | | | | |
| | | | | LPLLAL | | | | | | | | |
| | | | | LLLLCW | | | | | | | | |
| | | | | KYCA | | | | | | | | |
| 470 | NM_001318252.1: | NP_001305181.1: | 176 | VQKAEA | 1076 | HLA- | 23.18 | 0.153634394 | 2 | No | No | NA |
| | c.535del | p.Leu179Cys | | LMRELD | | B*08: | | | | | | |
| | | fsTer136 | | EEGSDP | | 01 | | | | | | |
| | | | | PLPGRA | | | | | | | | |
| | | | | QRIRQV | | | | | | | | |
| | | | | LQLLS | | | | | | | | |
| 513 | NM_031286.3: | NP_112576.1: | 479 | DISQDN | 1114 | HLA- | 111.3 | 8.755807231 | 2 | No | No | NA |
| | c.171del | p.Lys58Arg | | ALRDEM | | A*74: | | | | | | |
| | | fsTer33 | | RALAGN | | 01 | | | | | | |
| | | | | PKATPP | | | | | | | | |
| | | | | QIVNGD | | | | | | | | |
| | | | | QYCGDY | | | | | | | | |
| | | | | ELFV | | | | | | | | |
| 514 | NM_015330.4: | NP_056145.4: | 497 | KSGRYM | 1115 | HLA- | 35.14 | 3.432981809 | 2 | No | No | NA |
| | c.1508del | p.Arg503Leu | | ELEQRY | | C*12: | | | | | | |
| | | fsTer14 | | MDLAEN | | 03 | | | | | | |
| | | | | ARFERE | | | | | | | | |
| | | | | QLLGVQ | | | | | | | | |
| | | | | QHLSNT | | | | | | | | |
| | | | | LKMA | | | | | | | | |
| 515 | NM_032323.2: | NP_115699.1: | 505 | DLIVRC | 1116 | HLA- | 99.78 | 0.16475748 | 2 | No | No | NA |
| | c.487dup | p.Arg163Pro | | EAGEGE | | A*33: | | | | | | |
| | | fsTer9 | | CRTFMP | | 01 | | | | | | |
| | | | | PRVTHP | | | | | | | | |
| | | | | DPTERK | | | | | | | | |
| | | | | WAEAVV | | | | | | | | |
| | | | | RPPG | | | | | | | | |
| 516 | NM_001146694.1: | NP_001140166.1: | 538 | HVSQAQ | 1085 | HLA- | 393.64 | 25.68193545 | 2 | No | No | NA |
| | c.3110de1 | p.Ser1037Thr | | QETYLG | | C*16: | | | | | | |
| | | fsTer37 | | FWINSK | | 01 | | | | | | |
| | | | | KSQCNI | | | | | | | | |
| | | | | FLSGTY | | | | | | | | |
| 517 | NM_001310338.1: | NP_001297267.1: | 611 | EKYSNL | 1117 | HLA- | 350.66 | 19.27029849 | 2 | No | No | NA |
| | c.206del | p.Pro69Arg | | VQSVLS | | A*11: | | | | | | |
| | | fsTer14 | | SRGVAQ | | 01 | | | | | | |
| | | | | TPGSVE | | | | | | | | |
| | | | | EDALLC | | | | | | | | |
| | | | | GPVSKH | | | | | | | | |
| | | | | KLPN | | | | | | | | |
| 518 | NM_004888.3: | NP_004879.1: | 639 | KAKEAA | 1118 | HLA- | 248.43 | 0.311391637 | 2 | No | No | NA |
| | c.223225del | pLys75del | | ALGSRG | | A*11: | | | | | | |
| | | | | SCSTEV | | 01 | | | | | | |
| | | | | EKETQE | | | | | | | | |
| | | | | KMTILQ | | | | | | | | |
| | | | | TYFRQN | | | | | | | | |
| | | | | RDEV | | | | | | | | |
| 519 | NM_007214.4: | NP_009145.1: | 778 | GGWQQK | 1119 | HLA- | 415.63 | 0.687542718 | 2 | No | No | NA |
| | c.1586dup | pLys530Glu | | SKGPKK | | B*08: | | | | | | |
| | | fsTer30 | | TAKSKK | | 01 | | | | | | |
| | | | | KKPLKK | | | | | | | | |
| | | | | KPTPVL | | | | | | | | |
| | | | | LPQSKQ | | | | | | | | |
| | | | | QKQK | | | | | | | | |
| 520 | NM_003214.3: | NP_003205.2: | 854 | QIVSAS | 1120 | HLA- | 179.51 | 1.409435347 | 2 | No | No | NA |
| | c.454del | pGln152Arg | | VLQNKF | | B*53: | | | | | | |
| | | fsTer115 | | SPPSPL | | 01 | | | | | | |
| | | | | PQAVFS | | | | | | | | |
| | | | | TSSRFW | | | | | | | | |
| | | | | SSPPLL | | | | | | | | |
| | | | | GQQP | | | | | | | | |
| 521 | NM_001079818.1: | NP_001073286.1: | 1061 | LQRANR | 1121 | HLA- | 307.06 | 1.322184587 | 2 | No | No | NA |
| | c.276del | p.Pro93His | | TGGLYS | | C*12: | | | | | | |
| | | fsTer37 | | CDITAR | | 03 | | | | | | |
| | | | | GPCTRI | | | | | | | | |
| | | | | EFDNDA | | | | | | | | |
| | | | | DPTSES | | | | | | | | |
| | | | | KEDQ | | | | | | | | |
| 522 | NM_001122964.1: | NP_001116436.1: | 1086 | VEHHTY | 1122 | HLA- | 527.11 | 5.084704108 | 2 | No | No | NA |
| | c.1720_1722del | p.Arg574del | | HIKNYI | | C*12: | | | | | | |
| | | | | MNKDLL | | 03 | | | | | | |
| | | | | RRVLVL | | | | | | | | |
| | | | | MNSKHT | | | | | | | | |
| | | | | FLALCA | | | | | | | | |
| | | | | LRFM | | | | | | | | |
| 523 | NM_001002295.1: | NP_001002295.1: | 1521 | PITTYP | 1123 | HLA- | 16.01 | 0 | 2 | No | No | NA |
| | c.708_709insC | p.Ser237Gln | | PYVPEY | | C*16: | | | | | | |
| | | fsTer67 | | SSGLFP | | 01 | | | | | | |
| | | | | PSSLLG | | | | | | | | |
| | | | | GSPTGF | | | | | | | | |
| | | | | GCKSRP | | | | | | | | |
| | | | | KARS | | | | | | | | |
| 524 | NM_000706.4: | NP_000697.1: | 1535 | ITALLG | 1124 | HLA- | 10.5 | 0 | 2 | No | No | NA |
| | c.1052del | pPhe351Leu | | SLNSCC | | C*16: | | | | | | |
| | | fsTer19 | | NPWIYM | | 01 | | | | | | |
| | | | | FFSGHL | | | | | | | | |
| | | | | LQDCVQ | | | | | | | | |
| | | | | SFPCCQ | | | | | | | | |
| | | | | NMKE | | | | | | | | |
| 525 | NM_001146197.1: | NP_001139669.1: | 1571 | PHHDDI | 1125 | HLA- | 10.16 | 0 | 2 | Yes | No | NA |
| | c.21050del | p.Phe7017Leu | | NFYSER | | A*68: | | | | | | |
| | | fsTer25 | | KQNRPF | | 01 | | | | | | |
| | | | | FFACVP | | | | | | | | |
| | | | | ADSLEV | | | | | | | | |
| | | | | IPKTIR | | | | | | | | |
| | | | | WTIP | | | | | | | | |
| 526 | NM_198994.2: | NP_945345.2: | 1572 | ARQDLG | 1126 | HLA- | 28.77 | 0 | 2 | Yes | No | NA |
| | c.1078del | p.Gln360Arg | | PSYNGW | | A*11: | | | | | | |
| | | fsTer99 | | QVLDAT | | 01 | | | | | | |
| | | | | PQEESE | | | | | | | | |
| | | | | GVFRCG | | | | | | | | |
| | | | | PASVTA | | | | | | | | |
| | | | | IREG | | | | | | | | |
| 527 | NM_002725.3: | NP_002716.1: | 1633 | LPPGPP | 1127 | HLA- | 4.28 | 0 | 2 | No | No | NA |
| | c.242del | p.Pro81Leu | | SIFPDC | | B*53: | | | | | | |
| | | fsTer61 | | PRECYC | | 01 | | | | | | |
| | | | | PPDFPS | | | | | | | | |
| | | | | ALYCDS | | | | | | | | |
| | | | | RNLRKV | | | | | | | | |
| | | | | PVIP | | | | | | | | |
| 528 | NM_003285.2: | NP_003276.3: | 1635 | WFGKNC | 1128 | HLA- | 22.62 | 0 | 2 | No | No | NA |
| | c.636del | p.Val213Cys | | SEPYCP | | C*12: | | | | | | |
| | | fsTer52 | | LGCSSR | | 03 | | | | | | |
| | | | | GVCVDG | | | | | | | | |
| | | | | QCICDS | | | | | | | | |
| | | | | EYSGDD | | | | | | | | |
| | | | | CSEL | | | | | | | | |
| 529 | NM_001185024.1: | NP_001171953.1: | 1645 | RRGGDH | 1129 | HLA- | 4.61 | 0 | 2 | No | No | NA |
| | c.1374de1 | p.Thr459Arg | | VALQPL | | | | | | | | |
| | | fsTer177 | | RSEGGP | | | | | | | | |
| | | | | PTPHRS | | | | | | | | |
| | | | | IFAPHA | | | | | | | | |
| | | | | LPNRNG | | B*53: | | | | | | |
| | | | | SLSY | | 01 | | | | | | |
| 530 | NM_203468.2: | NP_982293.1: | 1646 | WVGRWF | 1130 | HLA- | 5.76 | 0 | 2 | No | No | NA |
| | c.610del | p.Gly204Val | | RPRKGT | | B*53: | | | | | | |
| | | fsTer171 | | LGAMDL | | 01 | | | | | | |
| | | | | GGASTQ | | | | | | | | |
| | | | | ITFETT | | | | | | | | |
| | | | | SPAEDR | | | | | | | | |
| | | | | ASEV | | | | | | | | |
| 531 | NM_138694.3: | NP_619639.3: | 1673 | TADEPM | 1131 | HLA- | 25.63 | 0 | 2 | No | No | NA |
| | c.4592del | p.Phe1531Leu | | VFVDDQ | | B*53: | | | | | | |
| | | fsTer61 | | LPCNVT | | 01 | | | | | | |
| | | | | FFNASH | | | | | | | | |
| | | | | VVCQTR | | | | | | | | |
| | | | | DLAPGP | | | | | | | | |
| | | | | HYLS | | | | | | | | |
| 532 | NM_020245.4: | NP_064630.2: | 1701 | CLKKGD | 1132 | HLA- | 11.74 | 0 | 2 | No | No | NA |
| | c.2647del | p.Leu883Trp | | FSLYPT | | B*53: | | | | | | |
| | | fsTer43 | | SVHYQT | | 01 | | | | | | |
| | | | | PLGYER | | | | | | | | |
| | | | | ITTFDS | | | | | | | | |
| | | | | SGNVEE | | | | | | | | |
| | | | | VCRP | | | | | | | | |
| 533 | NM_001013838.1: | NP_001013860.1: | 1723 | RAGRGG | 1133 | HLA- | 53.26 | 0 | 2 | No | No | NA |
| | c.1253_1254insC | p.Gln419Ala | | LGPPAG | | A*33: | | | | | | |
| | | fsTer112 | | VANSLP | | 01 | | | | | | |
| | | | | PQLFAA | | | | | | | | |
| | | | | VSRGCC | | | | | | | | |
| | | | | TSLTHL | | | | | | | | |
| | | | | DASR | | | | | | | | |
| 534 | NM_001145815.1: | NP_001139287.1: | 1739 | PDPLGP | 1134 | HLA- | 16.85 | 0 | 2 | No | No | NA |
| | c.1035_1036insC | p.Arg346Gln | | RSQPAC | | C*12: | | | | | | |
| | | fsTer56 | | QVAHDP | | 03 | | | | | | |
| | | | | PRACPL | | | | | | | | |
| | | | | CSQGTK | | | | | | | | |
| | | | | TLSGSI | | | | | | | | |
| | | | | APMD | | | | | | | | |
| 535 | NM_001256196.1: | NP_001243125.1: | 1762 | GQSFST | 1135 | HLA- | 52.13 | 0 | 2 | No | No | NA |
| | c.772del | p.Arg258Gly | | DAAGSR | | C*12: | | | | | | |
| | | fsTer37 | | GGSDGT | | 03 | | | | | | |
| | | | | PRGSPS | | | | | | | | |
| | | | | PASVSS | | | | | | | | |
| | | | | GRKSPH | | | | | | | | |
| | | | | SKSP | | | | | | | | |
| 536 | NM_001846.2: | NP_001837.2: | 1922 | QGRRGP | 1136 | HLA- | 184.44 | 0 | 2 | No | No | NA |
| | c.4275del | p.Gly1426Glu | | PGAPGE | | C*12: | | | | | | |
| | | fsTer33 | | MGPQGP | | 03 | | | | | | |
| | | | | PGEPGF | | | | | | | | |
| | | | | RGAPGK | | | | | | | | |
| | | | | AGPQGR | | | | | | | | |
| | | | | GGVS | | | | | | | | |
| 537 | NM_000431.2: | NP_000422.1: | 2011 | KORALP | 1137 | HLA- | 273.02 | 0 | 2 | No | No | NA |
| | c.417del | pAla141Arg | | SLDIVV | | B*53: | | | | | | |
| | | fsTer18 | | WSELPP | | 01 | | | | | | |
| | | | | GAGLGS | | | | | | | | |
| | | | | SAAYSV | | | | | | | | |
| | | | | CLAAAL | | | | | | | | |
| | | | | LTVC | | | | | | | | |
| 538 | NM_001184772.2: | NP_001171701.1: | 2059 | SSQLLT | 898 | HLA- | 225 | 0 | 2 | Yes | No | NA |
| | c.5264de1 | p.Pro1755Gln | | PAERPG | | B*53: | | | | | | |
| | | fsTer20 | | GLDDRS | | 01 | | | | | | |
| | | | | PPGSSE | | | | | | | | |
| | | | | TVELVR | | | | | | | | |
| | | | | YEPDLL | | | | | | | | |
| | | | | RLLG | | | | | | | | |
| 539 | NM_182557.2: | NP_872363.1: | 2070 | GPPGGA | 1138 | HLA- | 61.17 | 0 | 2 | No | No | NA |
| | c.1354del | p.Gln452Ser | | GEGGPP | | C*16: | | | | | | |
| | | fsTer11 | | AQAPPP | | 01 | | | | | | |
| | | | | PQQPPT | | | | | | | | |
| | | | | APPSGL | | | | | | | | |
| | | | | KKYEEP | | | | | | | | |
| | | | | LQSM | | | | | | | | |
| 540 | NM_001105556.1: | NP_001099026.1: | 2221 | HFIKPL | 1139 | HLA- | 22.42 | 0 | 2 | No | No | NA |
| | c.781del | p.Leu261Cys | | LLSEVL | | A*11: | | | | | | |
| | | fsTer34 | | AWEGPF | | 01 | | | | | | |
| | | | | PLSMEI | | | | | | | | |
| | | | | LEVPEG | | | | | | | | |
| | | | | RPIFLS | | | | | | | | |
| | | | | PWVG | | | | | | | | |
| 541 | NM_001306137.1: | NP_001293066.1: | 2253 | YFKKLV | 1140 | HLA- | 29.2 | 0 | 2 | No | No | NA |
| | c.1899de1 | p.Asp634Met | | LNKAIL | | | | | | | | |
| | | fsTer6 | | LQVIAK | | | | | | | | |
| | | | | KDDKYT | | | | | | | | |
| | | | | VNIQSV | | | | | | | | |
| | | | | EASENI | | C*12: | | | | | | |
| | | | | DVIS | | 03 | | | | | | |
| 542 | NM_016642.3: | NP_057726.4: | 2270 | ARLQTE | 1141 | HLA- | 343.16 | 0 | 2 | No | No | NA |
| | c.9862del | pGly3288Ala | | ACRLGQ | | C*12: | | | | | | |
| | | fsTer40 | | LHPAAP | | 03 | | | | | | |
| | | | | GGLAKV | | | | | | | | |
| | | | | QEAWAT | | | | | | | | |
| | | | | LQAKAQ | | | | | | | | |
| | | | | ERGQ | | | | | | | | |
| 543 | NM_181882.2: | NP_870998.2: | 2295 | VGGEGA | 1142 | HLA- | 29.03 | 0 | 2 | No | No | NA |
| | c.3823_3824del | p.Ser1275Thr | | EEQPPG | | A*11: | | | | | | |
| | | fsTer49 | | AERTFC | | 01 | | | | | | |
| | | | | LSLPDV | | | | | | | | |
| | | | | ELSPSG | | | | | | | | |
| | | | | GNHAEY | | | | | | | | |
| | | | | QVAE | | | | | | | | |
| 544 | NM_002536.2: | NP_002527.1: | 2355 | ASPTGD | 1143 | HLA- | 19.95 | 0 | 2 | No | No | NA |
| | c.1769del | p.Pro590Arg | | MAVGSP | | C*12: | | | | | | |
| | | fsTer118 | | LMQEVG | | 03 | | | | | | |
| | | | | SPKDPG | | | | | | | | |
| | | | | KSLPPV | | | | | | | | |
| | | | | PPMGLP | | | | | | | | |
| | | | | PPQE | | | | | | | | |
| 545 | NM_001130924.2: | NP_001124396.2: | 2364 | SEEAAT | 1144 | HLA- | 20.77 | 0 | 2 | No | No | NA |
| | c.1936de1 | p.Leu646Trp | | WRGRFG | | | | | | | | |
| | | fsTer3 | | PSLVRG | | | | | | | | |
| | | | | LLAVSL | | | | | | | | |
| | | | | AANALF | | | | | | | | |
| | | | | TSVFLY | | B*53: | | | | | | |
| | | | | QSLR | | 01 | | | | | | |
| 546 | NM_020064.3: | NP_064448.1: | 2392 | RILIHG | 1145 | HLA- | 29.29 | 0 | 2 | No | No | NA |
| | c.946del | pLeu316Trp | | LQGASE | | C*12: | | | | | | |
| | | fsTer184 | | PPPPLP | | 03 | | | | | | |
| | | | | PLAGVL | | | | | | | | |
| | | | | PRAAQP | | | | | | | | |
| | | | | R | | | | | | | | |
| 547 | NM_003119.2: | NP_003110.1: | 2420 | RFLQLG | 1146 | HLA- | 13.52 | 0 | 2 | No | No | NA |
| | c.1053del | pGly352Ala | | AKVPKG | | A*33: | | | | | | |
| | | fsTer87 | | ALLLGP | | 01 | | | | | | |
| | | | | PGCGKT | | | | | | | | |
| | | | | LLAKAV | | | | | | | | |
| | | | | ATEAQV | | | | | | | | |
| | | | | PFLA | | | | | | | | |
| 548 | NM_001317012.1: | NP_001303941.1: | 2463 | CPQDQS | 1147 | HLA- | 36.4 | 0 | 2 | No | No | NA |
| | c.836del | p.Asn279Met | | PDRVGT | | A*11: | | | | | | |
| | | fsTer55 | | EMEQVS | | 01 | | | | | | |
| | | | | KNEGCQ | | | | | | | | |
| | | | | AGAELE | | | | | | | | |
| | | | | ELSKKA | | | | | | | | |
| | | | | GPEE | | | | | | | | |
| 549 | NM_015174.1: | NP_055989.1: | 2505 | PTATGV | 1148 | HLA- | 29.53 | 0 | 2 | No | No | NA |
| | c.563del | pPro188Arg | | QPESSA | | B*53: | | | | | | |
| | | fsTer239 | | SIVTSY | | 01 | | | | | | |
| | | | | PPPSYN | | | | | | | | |
| | | | | PTCTAY | | | | | | | | |
| | | | | TAPSYP | | | | | | | | |
| | | | | NYDA | | | | | | | | |
| 550 | NM_002226.4: | NP_002217.3: | 2531 | SNGGTC | 1149 | HLA- | 54.82 | 0 | 2 | No | No | NA |
| | c.2220del | pGly741Ala | | YDSGDT | | C*12: | | | | | | |
| | | fsTer21 | | FRCACP | | 03 | | | | | | |
| | | | | PGWKGS | | | | | | | | |
| | | | | TCAVAK | | | | | | | | |
| | | | | NSSCLP | | | | | | | | |
| | | | | NPCV | | | | | | | | |
| 551 | NM_005211.3: | NP_005202.2: | 2576 | GATVTL | 1150 | HLA- | 92.3 | 0 | 2 | No | No | NA |
| | c.161del | p.Pro54His | | RCVGNG | | C*12: | | | | | | |
| | | fsTer58 | | SVEWDG | | 03 | | | | | | |
| | | | | PPSPHW | | | | | | | | |
| | | | | TLYSDG | | | | | | | | |
| | | | | SSSILS | | | | | | | | |
| | | | | TNNA | | | | | | | | |
| 552 | NM_006514.3: | NP_006505.3: | 2595 | SEDLAP | 1151 | HLA- | 53.28 | 0 | 2 | No | No | NA |
| | c.3218del | p.Val1073Ala | | SLGETW | | A*33: | | | | | | |
| | | fsTer20 | | KDESVP | | 01 | | | | | | |
| | | | | QVPAEG | | | | | | | | |
| | | | | VDDTSS | | | | | | | | |
| | | | | SEGSTV | | | | | | | | |
| | | | | DCLD | | | | | | | | |
| 553 | NM_138703.4: | NP_619648.1: | 2633 | IQATNA | 1152 | HLA- | 89. | 0 | 2 | Yes | No | NA |
| | c.133del | p.Gln45Ser | | SGSPTS | | B*53: | | | | | | |
| | | fsTer27 | | MLVVDA | | 01 | | | | | | |
| | | | | PQCPQA | | | | | | | | |
| | | | | PINSQC | | | | | | | | |
| | | | | VNTSQA | | | | | | | | |
| | | | | VQDP | | | | | | | | |
| 554 | NM_000033.3: | NP_000024.2: | 2650 | RAFSPK | 1153 | HLA- | 27.18 | 0 | 2 | No | No | NA |
| | c.832del | p.Glu278Ser | | FGELVA | | B*53: | | | | | | |
| | | fsTer58 | | EEARRK | | 01 | | | | | | |
| | | | | GELRYM | | | | | | | | |
| | | | | HSRVVA | | | | | | | | |
| | | | | NSEEIA | | | | | | | | |
| | | | | FYGG | | | | | | | | |
| 555 | NM_153274.2: | NP_695006.1: | 2697 | APAAQT | 1154 | HLA- | 78.82 | 0 | 2 | No | No | NA |
| | c.1268del | pPro423Arg | | PLLGRF | | B*53: | | | | | | |
| | | fsTer97 | | LGVGAP | | 01 | | | | | | |
| | | | | SPAISL | | | | | | | | |
| | | | | RNFGRV | | | | | | | | |
| | | | | RGTPRP | | | | | | | | |
| | | | | PHLL | | | | | | | | |
| 556 | NM_012285.2: | NP_036417.1: | 2808 | NVFEPK | 1155 | HLA- | 388.96 | 0 | 2 | No | No | NA |
| | c.641_642insG | p.Ser215Val | | PSVPEY | | A*11: | | | | | | |
| | | fsTer39 | | KVASVG | | 01 | | | | | | |
| | | | | GSRCLL | | | | | | | | |
| | | | | LHYSVS | | | | | | | | |
| | | | | KAIWDG | | | | | | | | |
| | | | | LILL | | | | | | | | |
| 557 | NM_001136265.1: | NP_001129737.1: | 2810 | METCRR | 1156 | HLA- | 133.72 | 0 | 2 | No | No | NA |
| | c.1464de1 | p.Ser489Ala | | LIKGSA | | A*11: | | | | | | |
| | | fsTer32 | | DRNSPS | | 01 | | | | | | |
| | | | | PSSVAS | | | | | | | | |
| | | | | SDSGST | | | | | | | | |
| | | | | DEIQDE | | | | | | | | |
| | | | | FERE | | | | | | | | |
| 558 | NM_203487.2: | NP_982354.1: | 2829 | FFRLIK | 1157 | HLA- | 93.61 | 0 | 2 | No | No | NA |
| | c.420del | p.Met141Cys | | IKIIVK | | C*12: | | | | | | |
| | | fsTer16 | | DTNDNA | | 03 | | | | | | |
| | | | | PMFPSP | | | | | | | | |
| | | | | VINISI | | | | | | | | |
| | | | | PENTLI | | | | | | | | |
| | | | | NSRF | | | | | | | | |
| 559 | NM_019589.2: | NP_062535.2: | 2875 | LQPHHL | 1158 | HLA- | 63.56 | 0 | 2 | No | No | NA |
| | c.284del | p.Gly95Ala | | PPPPLP | | B*53: | | | | | | |
| | | fsTer124 | | PPPVMP | | 01 | | | | | | |
| | | | | GGGYGD | | | | | | | | |
| | | | | WQPPPP | | | | | | | | |
| | | | | PMPPPP | | | | | | | | |
| | | | | GPAL | | | | | | | | |
| 560 | NM_004431.3: | NP_004422.2: | 2976 | KVRLEG | 1159 | HLA- | 43.85 | 0 | 2 | Yes | No | NA |
| | c.1379del | p.Pro460Arg | | RSTTSL | | A*11: | | | | | | |
| | | fsTer33 | | SVSWSI | | 01 | | | | | | |
| | | | | PPPQQS | | | | | | | | |
| | | | | RVWKYE | | | | | | | | |
| | | | | VTYRKK | | | | | | | | |
| | | | | GDSN | | | | | | | | |
| 561 | NM_001042724.1: | NP_001036189.1 | 2987 | AEEDED | 1160 | HLA- | 37.69 | 0 | 2 | No | No | NA |
| | c.1236de1 | :p.Ala413Arg | | LEGPPS | | A*11: | | | | | | |
| | | fsTer82 | | YKPPTP | | 01 | | | | | | |
| | | | | KAKLEA | | | | | | | | |
| | | | | QEMPSQ | | | | | | | | |
| | | | | LFTLGA | | | | | | | | |
| | | | | SEHS | | | | | | | | |
| 562 | NM_007186.5: | NP_009117.2: | 3014 | GERDTL | 1161 | HLA- | 185.42 | 0 | 2 | No | No | NA |
| | c.1382_1383del | pLeu461Gln | | AGQTVD | | A*11: | | | | | | |
| | | fsTer80 | | LQGEVD | | 01 | | | | | | |
| | | | | SLSKER | | | | | | | | |
| | | | | ELLQKA | | | | | | | | |
| | | | | REELRQ | | | | | | | | |
| | | | | QLEV | | | | | | | | |
| 563 | NM_001851.4: | NP_001842.3: | 3030 | TTDERG | 1162 | HLA- | 3.52 | 0 | 2 | No | No | NA |
| | c.848del | pPro283Leu | | PPGEQG | | A*68: | | | | | | |
| | | fsTer45 | | PPGPPG | | 01 | | | | | | |
| | | | | PPGVPG | | | | | | | | |
| | | | | IDGIDG | | | | | | | | |
| | | | | DRGPKG | | | | | | | | |
| | | | | PPGP | | | | | | | | |
| 564 | NM_138300.3: | NP_612157.1: | 3059 | RQPPPF | 1163 | HLA- | 147.32 | 0 | 2 | No | No | NA |
| | c.448dup | p.Gln150Pro | | PPNPMG | | A*11: | | | | | | |
| | | fsTer27 | | PAFNMP | | 01 | | | | | | |
| | | | | PQGPGY | | | | | | | | |
| | | | | PPPGNM | | | | | | | | |
| | | | | NFPSQP | | | | | | | | |
| | | | | FNQP | | | | | | | | |
| 565 | NM_005994.3: | NP_005985.3: | 3075 | CKPERD | 1164 | HLA- | 408.47 | 0 | 2 | No | No | NA |
| | c.987del | p.Ala330Arg | | GAESDA | | A*74: | | | | | | |
| | | fsTer38 | | SSCDPP | | 01 | | | | | | |
| | | | | PAREPP | | | | | | | | |
| | | | | TSPGAA | | | | | | | | |
| | | | | PSPLRL | | | | | | | | |
| | | | | HRAR | | | | | | | | |
| 566 | NM_014727.1: | NP_055542.1: | 3198 | KHKTTP | 1165 | HLA- | 303.52 | 0 | 2 | No | No | NA |
| | c.521del | p.Pro174Gln | | LPPPRL | | C*12: | | | | | | |
| | | fsTer20 | | ADVAPT | | 03 | | | | | | |
| | | | | PPKTPA | | | | | | | | |
| | | | | RKRGEE | | | | | | | | |
| | | | | GTERMV | | | | | | | | |
| | | | | QALT | | | | | | | | |
| 567 | NM_001271223.2: | NP_001258152.2: | 3214 | PSSEAC | 1166 | HLA- | 10.87 | 0 | 2 | No | No | NA |
| | c.23848del | p.Ala7950Pro | | GEAQRL | | B*07: | | | | | | |
| | | fsTer79 | | PSAPSG | | 02 | | | | | | |
| | | | | GAPIRD | | | | | | | | |
| | | | | MGHPQG | | | | | | | | |
| | | | | SKQLPS | | | | | | | | |
| | | | | TGGH | | | | | | | | |
| 568 | NM_004947.4: | NP_004938.1: | 3229 | KGHYSL | 877 | HLA- | 16.83 | 0 | 2 | Yes | Yes | No |
| | c.5555del | pPro1852Gln | | HFDAFH | | A*68: | | | | | | |
| | | fsTer45 | | HPLGDT | | 01 | | | | | | |
| | | | | PPALPA | | | | | | | | |
| | | | | RTLRKS | | | | | | | | |
| | | | | PLHPIP | | | | | | | | |
| | | | | ASPT | | | | | | | | |
| 569 | NM_001145168.1: | NP_001138640.1: | 3279 | TDGSPH | 1167 | HLA- | 107.53 | 0 | 2 | Yes | No | NA |
| | c.2183de1 | p.Gly728Val | | CVFWDH | | C*12: | | | | | | |
| | | fsTer46 | | SLFQGR | | 03 | | | | | | |
| | | | | GGWSKE | | | | | | | | |
| | | | | GCQAQV | | | | | | | | |
| | | | | ASASPT | | | | | | | | |
| | | | | AQCL | | | | | | | | |
| 570 | NM_001271875.1: | NP_001258804.1: | 3418 | SGGGGT | 1168 | HLA- | 165.61 | 0 | 2 | No | No | NA |
| | c.91del | p.Gln31Arg | | AGARGG | | B*53: | | | | | | |
| | | fsTer57 | | GGGTAA | | 01 | | | | | | |
| | | | | PQELNN | | | | | | | | |
| | | | | SRPARQ | | | | | | | | |
| | | | | VRRLEF | | | | | | | | |
| | | | | NQAM | | | | | | | | |
| 571 | NM_021954.3: | NP_068773.2: | 3555 | ERQPPA | 1169 | HLA- | 134.62 | 0 | 2 | No | No | NA |
| | c.1056del | p.Ser353Ala | | LKAYPA | | A*33: | | | | | | |
| | | fsTer46 | | ASTPAA | | 01 | | | | | | |
| | | | | PSPVGS | | | | | | | | |
| | | | | SSPPLA | | | | | | | | |
| | | | | HEAEAG | | | | | | | | |
| | | | | AAPL | | | | | | | | |
| 572 | NM_005811.3: | NP_005802.1: | 3605 | PKRYKA | 1170 | HLA- | 159.34 | 0 | 2 | No | No | NA |
| | c.1056del | pLys352Asn | | NYCSGQ | | C*12: | | | | | | |
| | | fsTer131 | | CEYMFM | | 03 | | | | | | |
| | | | | QKYPHT | | | | | | | | |
| | | | | HLVQQA | | | | | | | | |
| | | | | NPRGSA | | | | | | | | |
| | | | | GPCC | | | | | | | | |
| 573 | NM_173566.2: | NP_775837.2: | 3633 | GLDELD | 1171 | HLA- | 222.46 | 0 | 2 | No | No | NA |
| | c.5987del | pPro1996Gln | | GVKAAC | | A*11: | | | | | | |
| | | fsTer42 | | PCPQSS | | 01 | | | | | | |
| | | | | PPEQKE | | | | | | | | |
| | | | | AEPEKR | | | | | | | | |
| | | | | PKKVSQ | | | | | | | | |
| | | | | IRIR | | | | | | | | |
| 574 | NM_001271875.1: | NP_001258804.1: | 3647 | MTSLFR | 1172 | HLA- | 204.08 | 0 | 2 | Yes | No | NA |
| | c.44del | p.Gly15Val | | RSSSGS | | A*11: | | | | | | |
| | | fsTer73 | | GGGGTA | | 01 | | | | | | |
| | | | | GARGGG | | | | | | | | |
| | | | | GGTAAP | | | | | | | | |
| | | | | QELNN | | | | | | | | |
| 575 | NM_030661.4: | NP_109377.1: | 3670 | PPPPQK | 1173 | HLA- | 32.16 | 0 | 2 | No | No | NA |
| | c.1020del | p.Asp341Thr | | RYTAAG | | C*12: | | | | | | |
| | | fsTer35 | | AGAGGT | | 03 | | | | | | |
| | | | | PDYDPH | | | | | | | | |
| | | | | AHGLQG | | | | | | | | |
| | | | | NGSYGT | | | | | | | | |
| | | | | PHIQ | | | | | | | | |
| 576 | NM_014614.2: | NP_055429.2: | 3730 | LMNLSA | 1174 | HLA- | 145.44 | 0 | 2 | No | No | NA |
| | c.5412_5413del | p.Lys1804Asn | | HLNDPQ | | C*12: | | | | | | |
| | | fsTer11 | | PIEMTV | | 03 | | | | | | |
| | | | | KKTLSN | | | | | | | | |
| | | | | FRRTHH | | | | | | | | |
| | | | | DNWQEH | | | | | | | | |
| | | | | KQQF | | | | | | | | |
| 577 | NM_020700.1: | NP_065751.1: | 3860 | TPANSR | 1175 | HLA- | 178.69 | 0 | 2 | No | No | NA |
| | c.652dup | p.Ala218Gly | | TLTRAA | | B*53: | | | | | | |
| | | fsTer21 | | SLRGGV | | 01 | | | | | | |
| | | | | GAPGSP | | | | | | | | |
| | | | | STPPTR | | | | | | | | |
| | | | | FFTEKK | | | | | | | | |
| | | | | IPHE | | | | | | | | |
| 578 | NM_018273.2: | NP_060743.2: | 3894 | ELWLRL | 1176 | HLA- | 445.99 | 0 | 2 | No | No | NA |
| | c.66_67insG | pVal23Gly | | RGKGLA | | A*74: | | | | | | |
| | | fsTer105 | | MLHVTR | | 01 | | | | | | |
| | | | | GVWGSR | | | | | | | | |
| | | | | VRVWPL | | | | | | | | |
| | | | | LPALLG | | | | | | | | |
| | | | | PPRA | | | | | | | | |
| 579 | NM_198517.3: | NP_940919.1: | 4106 | RGACPG | 954 | HLA- | 220.38 | 0 | 2 | Yes | No | NA |
| | c.960del | pAla321Arg | | LLETLG | | A*33: | | | | | | |
| | | fsTer100 | | ALRAIP | | 01 | | | | | | |
| | | | | PAQLQE | | | | | | | | |
| | | | | EAFMSQ | | | | | | | | |
| | | | | VHSVVL | | | | | | | | |
| | | | | SERD | | | | | | | | |
| 580 | NM_014909.4: | NP_055724.1: | 4161 | RYIREL | 1177 | HLA- | 266.97 | 0 | 2 | No | No | NA |
| | c.437_439del | pLys146del | | QYNHTG | | A*33: | | | | | | |
| | | | | TQFFEI | | 01 | | | | | | |
| | | | | KKSRPL | | | | | | | | |
| | | | | TGLMDL | | | | | | | | |
| | | | | AKEMTK | | | | | | | | |
| | | | | EALP | | | | | | | | |
| 581 | NM_172238.3: | NP_758438.2: | 4512 | GSQYGM | 1178 | HLA- | 312.69 | 0 | 2 | No | No | NA |
| | c.507del | pLeu170Trp | | HPDQRL | | B*53: | | | | | | |
| | | fsTer27 | | LPGPSL | | 01 | | | | | | |
| | | | | GLAAAG | | | | | | | | |
| | | | | ADDLQG | | | | | | | | |
| | | | | SVEAQC | | | | | | | | |
| | | | | GLVL | | | | | | | | |
| 582 | NM_001029882.2: | NP_001025053.1: | 4520 | VCSSPD | 1179 | HLA- | 335.61 | 0 | 2 | No | No | NA |
| | c.99del | p.Thr34Pro | | YLREPK | | A*33: | | | | | | |
| | | fsTer83 | | YYPGGP | | 01 | | | | | | |
| | | | | PTPRPL | | | | | | | | |
| | | | | LPTRPP | | | | | | | | |
| | | | | ASPPDK | | | | | | | | |
| | | | | AFST | | | | | | | | |
| 583 | NM_018357.2: | NP_060827.2: | 4556 | TPQKNG | 1180 | HLA- | 371.96 | 0 | 2 | No | No | NA |
| | c.663del | p.Phe221Leu | | RVQEKV | | B*53: | | | | | | |
| | | fsTer56 | | MEHLLK | | 01 | | | | | | |
| | | | | LFGTFG | | | | | | | | |
| | | | | VISSVR | | | | | | | | |
| | | | | ILKPGR | | | | | | | | |
| | | | | ELPP | | | | | | | | |
| 584 | NM_012293.1: | NP_036425.1: | 4957 | HCSNVC | 1181 | HLA- | 230.37 | 0 | 2 | No | No | NA |
| | c.2592del | p.Asn865Met | | SNDPPC | | C*12: | | | | | | |
| | | fsTer25 | | FSVMIP | | 03 | | | | | | |
| | | | | PNDSRA | | | | | | | | |
| | | | | RSGARC | | | | | | | | |
| | | | | MFFVRS | | | | | | | | |
| | | | | SPVC | | | | | | | | |
| 585 | NM_133448.2: | NP_597705.2: | 5019 | DLGLCV | 901 | HLA- | 342.49 | 0 | 2 | No | No | NA |
| | c.618del | p.Thr207Arg | | AELELL | | B*53: | | | | | | |
| | | fsTer75 | | SSWFSP | | 01 | | | | | | |
| | | | | PTVVAG | | | | | | | | |
| | | | | RRKSVD | | | | | | | | |
| | | | | QPEGTP | | | | | | | | |
| | | | | VELY | | | | | | | | |
| 586 | NM_000243.2: | NP_000234.1: | 5037 | EVRLRR | 1182 | HLA- | 643.06 | 0 | 2 | No | No | NA |
| | c.655del | p.Gly219Ala | | NASSAG | | A*74: | | | | | | |
| | | fsTer43 | | RLQGLA | | 01 | | | | | | |
| | | | | GGAPGQ | | | | | | | | |
| | | | | KECRPF | | | | | | | | |
| | | | | EVYLPS | | | | | | | | |
| | | | | GKMR | | | | | | | | |
| 587 | NM_001039753.2: | NP_001034842.2: | 5191 | NPSIRA | 1183 | HLA- | 353.07 | 0 | 2 | No | No | NA |
| | c.2930del | p.Asn977Met | | ITLGHG | | A*74: | | | | | | |
| | | fsTer13 | | HILVGT | | 01 | | | | | | |
| | | | | KNGEIL | | | | | | | | |
| | | | | EIDKSG | | | | | | | | |
| | | | | PMTLLV | | | | | | | | |
| | | | | QGHM | | | | | | | | |
| 588 | NM_001783.3: | NP_001774.1: | 5332 | NESYQQ | 1184 | HLA- | 153.03 | 0 | 2 | No | No | NA |
| | c.390del | p.Arg131Gly | | SCGTYL | | A*11: | | | | | | |
| | | fsTer61 | | RVRQPP | | 01 | | | | | | |
| | | | | PRPFLD | | | | | | | | |
| | | | | MGEGTK | | | | | | | | |
| | | | | NRIITA | | | | | | | | |
| | | | | EGII | | | | | | | | |
| 589 | NM_016274.4: | NP_057358.2: | 5544 | ASSLSR | 1185 | HLA- | 478.42 | 0 | 2 | No | No | NA |
| | c.766del | p.Gln256Arg | | PWEKTD | | A*74: | | | | | | |
| | | fsTer6 | | KGATYT | | 01 | | | | | | |
| | | | | PQAPKK | | | | | | | | |
| | | | | LTPTEK | | | | | | | | |
| | | | | GRCASL | | | | | | | | |
| | | | | EEIL | | | | | | | | |
| 590 | NM_032836.2: | NP_116225.2: | 5776 | MDDVPA | 1186 | HLA- | 380.41 | 0 | 2 | No | No | NA |
| | c.15del | p.Ala6Pro | | PTPAPA | | C*12: | | | | | | |
| | | fsTer61 | | PPAAAA | | 03 | | | | | | |
| | | | | PRVPFH | | | | | | | | |
| | | | | CS | | | | | | | | |
| 591 | NM_014943.3: | NP_055758.1: | 6090 | KLRDSM | 1187 | HLA- | 352.11 | 0 | 2 | No | No | NA |
| | c.1800del | p.Gly601Ala | | EQAVLD | | C*12: | | | | | | |
| | | fsTer22 | | SMGSGK | | 03 | | | | | | |
| | | | | KGQDVG | | | | | | | | |
| | | | | APNGAL | | | | | | | | |
| | | | | SRLDQL | | | | | | | | |
| | | | | SGAQ | | | | | | | | |

SUPPLEMENTARY TABLE 10
List of the Top 100 most immunogenic predicted MHC-II neoAgs, with higher immunogenicity, obtained from
the computational methods in the validation set.

| | | | | | | | Reference | | Altered | | Pep- |
|---|---|---|---|---|---|---|---|---|---|---|---|
| Mutant | | | | | | Micro- | MS | | MS | Number | tide |
| Epitope | SEQ ID | Gene | Chromo- | | | satellite | lengths | Variant | Length | deleted | Len- |
| Sequence | NO | Name | some | Start | Stop | motif | (repeats) | Type | (repeats) | nucleotides | gth |
| CQKKLMLLRL | 592 | SEC31A | chr4 | 83785564 | 83785565 | T | 9 | FS | 8 | −1 | 15 |
| NLRKM | | | | | | | | | | | |
| DSQHVNLFLT | 593 | CEP162 | chr6 | 84896232 | 84896233 | T | 8 | FS | 7 | −1 | 15 |
| KMMRM | | | | | | | | | | | |
| TYKVKAAASA | 594 | TCF7L2 | chr10 | 114925316 | 114925317 | A | 9 | FS | 8 | −1 | 15 |
| HPLQM | | | | | | | | | | | |
| QLFVMSDTTY | 595 | TTK | chr6 | 80751896 | 80751897 | A | 9 | FS | 8 | −1 | 15 |
| KIYWT | | | | | | | | | | | |
| EPVLSSLTSL | 596 | RNF43 | chr17 | 56448297 | 56448298 | G | 6 | FS | 5 | −1 | 15 |
| RIELL | | | | | | | | | | | |
| RLIIQNLKSV | 597 | TGS1 | chr8 | 56711598 | 56711599 | A | 6 | FS | 5 | −1 | 15 |
| RAYLQ | | | | | | | | | | | |
| LGGLIKLKLN | 598 | ALMS1 | chr2 | 73800080 | 73800081 | A | 7 | FS | 6 | −1 | 15 |
| RLNLI | | | | | | | | | | | |
| IISLFITKAY | 599 | ZBTB41 | chr1 | 197145702 | 197145703 | T | 7 | FS | 6 | −1 | 15 |
| TLERK | | | | | | | | | | | |
| SILLHLIVLN | 600 | SREK1IP1 | chr5 | 64020297 | 64020298 | A | 7 | FS | 6 | −1 | 15 |
| SPESN | | | | | | | | | | | |
| SFLFLRQRAT | 601 | EPC2 | chr2 | 149447828 | 149447829 | A | 8 | FS | 7 | −1 | 15 |
| STITI | | | | | | | | | | | |
| ELGILTSFGV | 602 | YLPM1 | chr14 | 75283720 | 75283721 | T | 6 | FS | 5 | −1 | 15 |
| QQKPR | | | | | | | | | | | |
| GNINLTFFTT | 603 | LIPT1 | chr2 | 99778780 | 99778781 | A | 8 | FS | 7 | −1 | 15 |
| KKSMI | | | | | | | | | | | |
| QARLCLIVSR | 604 | MSH3 | chr5 | 79970914 | 79970915 | A | 8 | FS | 7 | −1 | 15 |
| TLLLV | | | | | | | | | | | |
| VSFWTLLPTT | 605 | SLC3A2 | chr11 | 62649528 | 62649529 | A | 8 | FS | 7 | −1 | 15 |
| GVRTR | | | | | | | | | | | |
| RSLTCVLSVG | 606 | ZNF585B | chr19 | 37676332 | 37676333 | A | 6 | FS | 5 | −1 | 15 |
| RPLAT | | | | | | | | | | | |
| RPRLLLARTS | 607 | MED19 | chr11 | 57479666 | 57479667 | G | 13 | FS | 12 | −1 | 15 |
| QELAV | | | | | | | | | | | |
| GKLMHVLYFS | 608 | KDM4C | chr9 | 7170005 | 7170006 | A | 7 | FS | ins G | −1 | 15 |
| SNEVT | | | | | | | | | | | |
| LQEWSRAISG | 609 | TNRC6A | chr16 | 24802365 | 24802366 | G | 7 | FS | 6 | −1 | 15 |
| GIAKR | | | | | | | | | | | |
| RGPLTSAPSA | 610 | ZBTB20 | chr3 | 114058002 | 114058003 | G | 7 | FS | 6 | −1 | 15 |
| QQSLT | | | | | | | | | | | |
| KKNLCLLKSG | 611 | ANTXR2 | chr4 | 80905989 | 80905990 | C | 8 | FS | 7 | −1 | 15 |
| QLWML | | | | | | | | | | | |
| KTEIQLTMND | 612 | BMPR2 | chr2 | 203420129 | 203420130 | A | 7 | FS | 6 | −1 | 15 |
| SKHKL | | | | | | | | | | | |
| QKGILMLQNC | 613 | SETD5 | chr3 | 9486784 | 9486785 | A | 6 | FS | 5 | −1 | 15 |
| HSYHL | | | | | | | | | | | |
| PGMLFFFATW | 614 | FBXO34 | chr14 | 55818554 | 55818554 | T | 8 | FS | 7 | 1 | 15 |
| SALVR | | | | | | | | | | | |
| KKKGLMTLSK | 615 | RNPC3 | chr1 | 104076466 | 104076467 | A | 12 | FS | 11 | −1 | 15 |
| MIKKK | | | | | | | | | | | |
| DQKILLLLEE | 616 | AKAP13 | chr15 | 86273791 | 86273794 | GA | 5 | FS | 4 | −2 | 15 |
| KMIFR | | | | | | | | | | | |
| GTALIVHMTI | 617 | PPRC1 | chr10 | 103907123 | 103907124 | C | 13 | FS | 12 | −1 | 15 |
| TKGKE | | | | | | | | | | | |
| VKPRKLTKVR | 618 | ATM | chr11 | 108121536 | 108121536 | G | 4 | FS | 5 | 1 | 15 |
| FIKTL | | | | | | | | | | | |
| LIWKRVFILL | 619 | SGO1 | chr3 | 20216067 | 20216068 | T | 7 | FS | 6 | −1 | 15 |
| LSDKK | | | | | | | | | | | |
| IVHFLLFKTS | 620 | SLFN13 | chr17 | 33771843 | 33771843 | T | 5 | FS | 6 | 1 | 15 |
| GRVQH | | | | | | | | | | | |
| PLRISLSLSN | 621 | RBM33 | chr7 | 155531072 | 155531072 | CA | 6 | FS | 7 | 1 | 15 |
| LSNSP | | | | | | | | | | | |
| VAWFYKSLWA | 622 | SLC28A2 | chr15 | 45558287 | 45558288 | T | 5 | FS | 4 | −1 | 15 |
| PLLQR | | | | | | | | | | | |
| EKKLKKTPSL | 623 | NSD3 | chr8 | 38146944 | 38146944 | A | 7 | FS | 8 | 1 | 15 |
| QTHQS | | | | | | | | | | | |
| MYSIRMENST | 624 | STAT5A | chr17 | 40461419 | 40461420 | G | 5 | FS | 4 | −1 | 15 |
| WMRPW | | | | | | | | | | | |
| GKNGILEDSQ | 625 | ZC3H13 | chr13 | 46543501 | 46543501 | A | 6 | FS | 7 | 1 | 15 |
| KKRRY | | | | | | | | | | | |
| WLKKKMLKNV | 626 | CCDC34 | chr11 | 27362972 | 27362973 | T | 8 | FS | 7 | −1 | 15 |
| RGRRK | | | | | | | | | | | |
| GSFRFFGSRM | 627 | KIAA0100 | chr17 | 26971122 | 26971123 | T | 7 | FS | 6 | −1 | 17 |
| SVLSFSN | | | | | | | | | | | |
| VGKLVTLRNV | 628 | SUGP2 | chr19 | 19136393 | 19136394 | A | 8 | FS | 7 | −1 | 15 |
| STKKY | | | | | | | | | | | |
| RMQLCTQLAR | 629 | RNF43 | chr17 | 56435160 | 56435161 | G | 7 | FS | 6 | −1 | 15 |
| FFPIT | | | | | | | | | | | |
| GTFMVIDCLS | 630 | MYO3B | chr2 | 171509586 | 171509587 | T | 7 | FS | 6 | −1 | 15 |
| LRKKA | | | | | | | | | | | |
| LKTLFHLRGI | 631 | CDC16 | chr13 | 115037719 | 115037720 | A | 6 | FS | 5 | −1 | 15 |
| SGNLK | | | | | | | | | | | |
| IWNRIEPAST | 632 | GART | chr21 | 34882121 | 34882121 | A | 9 | FS | 10 | 1 | 15 |
| YRQYS | | | | | | | | | | | |
| FWQICHIKKH | 633 | SLC35F5 | chr2 | 114500276 | 114500277 | T | 10 | FS | 9 | −1 | 15 |
| FQTHK | | | | | | | | | | | |
| NEWVKSDQVK | 634 | AKAP7 | chr6 | 131481275 | 131481276 | A | 8 | FS | 7 | −1 | 15 |
| KRKKR | | | | | | | | | | | |
| SIPWAPTSSR | 635 | C1RL | chr12 | 7249399 | 7249400 | C | 5 | FS | 4 | −1 | 15 |
| SVCPI | | | | | | | | | | | |
| ELRHVVPAPA | 636 | IGSF9B | chr11 | 133790744 | 133790744 | C | 6 | FS | 7 | 1 | 15 |
| HRGAL | | | | | | | | | | | |
| WGFLHLVSPS | 637 | SLC39A8 | chr4 | 103189224 | 103189224 | A | 7 | FS | 8 | 1 | 15 |
| GTQYF | | | | | | | | | | | |
| LPTLRPTRPL | 638 | NFIC | chr19 | 3453852 | 3453853 | C | 6 | FS | 5 | −1 | 15 |
| QTVPL | | | | | | | | | | | |
| PAAYLLGLPG | 639 | RNF43 | chr17 | 56436054 | 56436055 | G | 3 | FS | 2 | −1 | 15 |
| VQWLG | | | | | | | | | | | |
| LFFFVRQWGV | 640 | ERVMER34-1 | chr4 | 53610788 | 53610788 | T | 8 | FS | 9 | 1 | 15 |
| QRVST | | | | | | | | | | | |
| SKMYTTSMAM | 641 | ARID1A | chr1 | 27100175 | 27100176 | C | 6 | FS | 5 | −1 | 15 |
| PILPL | | | | | | | | | | | |
| DIGEVSQFLT | 642 | MET | chr7 | 116418873 | 116418876 | CAT | 2 | FS | 1 | −2 | 15 |
| EGIMK | | | | | | | | | | | |
| PLMIKNRISP | 643 | SYNE2 | chr14 | 64450477 | 64450478 | A | 7 | FS | 6 | −1 | 15 |
| LLTIL | | | | | | | | | | | |
| YFINFIYLAK | 644 | ERMP1 | chr9 | 5801291 | 5801292 | A | 8 | FS | 7 | −1 | 15 |
| STKKP | | | | | | | | | | | |
| VLLQMFLRGL | 645 | BARD1 | chr2 | 215646084 | 215646085 | A | 7 | FS | 6 | −1 | 15 |
| KRLLQ | | | | | | | | | | | |
| LWELRFHQDR | 646 | NOL4L | chr20 | 31041555 | 31041555 | C | 7 | FS | 8 | 1 | 15 |
| GQRLP | | | | | | | | | | | |
| RPLKCMATLP | 647 | ZNF573 | chr19 | 38229650 | 38229651 | A | 6 | FS | 5 | −1 | 15 |
| NIRKF | | | | | | | | | | | |
| KEKKKNLAIV | 648 | MLH3 | chr14 | 75514603 | 75514604 | T | 9 | FS | 8 | −1 | 16 |
| EEEMFL | | | | | | | | | | | |
| EPGVLAAAAE | 649 | MRPL54 | chr19 | 3767280 | 3767280 | C | 4 | FS | 5 | 1 | 14 |
| TEHL | | | | | | | | | | | |
| YKKKAAICTT | 650 | RNF103 | chr2 | 86831014 | 86831014 | A | 8 | FS | 9 | 1 | 15 |
| PALVK | | | | | | | | | | | |
| TLETLVKDLK | 651 | RBM43 | chr2 | 152108087 | 152108088 | T | 10 | FS | 9 | −1 | 15 |
| KKSRV | | | | | | | | | | | |
| HVSVYPKRSF | 652 | ST7L | chr1 | 113084681 | 113084682 | A | 6 | FS | 5 | −1 | 15 |
| LCSSI | | | | | | | | | | | |
| RIRKKKVKSS | 653 | TDRD6 | chr6 | 46659362 | 46659363 | A | 7 | FS | 6 | −1 | 15 |
| VLOLK | | | | | | | | | | | |
| QPRLMILCSC | 654 | CDH1 | chr16 | 68867215 | 68867216 | C | 4 | FS | 3 | −1 | 15 |
| LTMKE | | | | | | | | | | | |
| RIAFGMMSMA | 655 | PRMT3 | chr11 | 20483595 | 20483596 | T | 5 | FS | 4 | −1 | 15 |
| SRCPA | | | | | | | | | | | |
| GLFYLLFCSK | 656 | RNF128 | chrX | 106016280 | 106016280 | T | 7 | FS | 8 | 1 | 15 |
| ATECK | | | | | | | | | | | |
| LVQSVLSSRG | 657 | MGME1 | chr20 | 17950704 | 17950705 | G | 4 | FS | 3 | −1 | 15 |
| VAQTR | | | | | | | | | | | |
| FPLAEKVKAL | 658 | RPLP0 | chr12 | 120634723 | 120634726 | CCT | 2 | FS | 1 | −2 | 17 |
| ADPSAFV | | | | | | | | | | | |
| AWHFSRAATE | 659 | ITGB4 | chr17 | 73733649 | 73733650 | C | 5 | FS | 4 | −1 | 15 |
| VTWWA | | | | | | | | | | | |
| RNSEITMQQI | 660 | ZMAT3 | chr3 | 178748779 | 178748780 | A | 5 | FS | 4 | −1 | 15 |
| AVLLL | | | | | | | | | | | |
| LSLIMLAQAQ | 661 | PDCD6IP | chr3 | 33866810 | 33866810 | T | 7 | FS | 8 | 1 | 15 |
| EVFFF | | | | | | | | | | | |
| QEYLPLEFHK | 662 | YLPM1 | chr14 | 75248387 | 75248388 | C | 5 | FS | 4 | −1 | 15 |
| GYLLS | | | | | | | | | | | |
| VTNFLKATGL | 663 | NEPRO | chr3 | 112727097 | 112727098 | A | 6 | FS | 5 | −1 | 15 |
| NIWKL | | | | | | | | | | | |
| GFELLRKNGL | 664 | GPATCH1 | chr19 | 33585104 | 33585104 | T | 8 | FS | 9 | 1 | 15 |
| ERRTR | | | | | | | | | | | |
| FSGICYLLSS | 665 | SLC16A4 | chr1 | 110906426 | 110906427 | T | 9 | FS | 8 | −1 | 15 |
| VSFFL | | | | | | | | | | | |
| PGILQQKMQF | 666 | ANKRD12 | chr18 | 9257835 | 9257836 | A | 7 | FS | 6 | −1 | 15 |
| RLLVL | | | | | | | | | | | |
| ATTIRLCWKA | 667 | DENND4B | chr1 | 153908615 | 153908616 | NA | NA | FS | NA | −1 | 15 |
| SGRLA | | | | | | | | | | | |
| QRASALLASC | 668 | FBXO34 | chr14 | 55817785 | 55817785 | A | 6 | FS | 7 | 1 | 15 |
| SKKLH | | | | | | | | | | | |
| ESAVTALVPP | 669 | KIAA1549 | chr7 | 138602055 | 138602055 | C | 6 | FS | 8 | 1 | 15 |
| PALSL | | | | | | | | | | | |
| ALPRIHNMSK | 670 | KIAA1211 | chr4 | 57179502 | 57179503 | A | 7 | FS | 6 | −1 | 15 |
| AALRV | | | | | | | | | | | |
| ELQLSVLSAE | 671 | PIK3C2A | chr11 | 17111375 | 17111376 | T | 6 | FS | 5 | −1 | 17 |
| SLRENFS | | | | | | | | | | | |
| VSGWVVVKSE | 672 | TIAL1 | chr10 | 121339505 | 121339509 | NA | NA | FS | NA | −3 | 15 |
| PIGPL | | | | | | | | | | | |
| RHRLNDIMTA | 673 | DEPDC7 | chr11 | 33047307 | 33047308 | A | 4 | FS | 3 | −1 | 15 |
| LLVQK | | | | | | | | | | | |
| PRRKTWKMRK | 674 | THAP5 | chr7 | 108205525 | 108205526 | T | 9 | FS | 8 | −1 | 15 |
| KYAQK | | | | | | | | | | | |
| YLIKLLSRDL | 675 | CHD1 | chr5 | 98206408 | 98206409 | A | 7 | FS | 6 | −1 | 15 |
| AKKKL | | | | | | | | | | | |
| PYLNTTGYPA | 676 | APC | chr5 | 112173779 | 112173780 | NA | NA | FS | NA | −1 | 15 |
| PLHQE | | | | | | | | | | | |
| QFVISPPALR | 677 | AHCTF1 | chr1 | 247007130 | 247007131 | T | 7 | FS | 6 | −1 | 15 |
| SRQKT | | | | | | | | | | | |
| QLGSGSSEAS | 678 | KMT2D | chr12 | 49426112 | 49426115 | TCT | 2 | FS | 1 | −2 | 15 |
| SVPHL | | | | | | | | | | | |
| PYFATYKAKM | 679 | C5orf42 | chr5 | 37182874 | 37182875 | T | 6 | FS | 5 | −1 | 15 |
| PLLRW | | | | | | | | | | | |
| FCKIKVSSAI | 680 | TTF2 | chr1 | 117633228 | 117633228 | T | 6 | FS | 7 | 1 | 15 |
| LSKKT | | | | | | | | | | | |
| AKVLLVRLKK | 681 | PRKCI | chr3 | 169998127 | 169998127 | A | 8 | FS | 9 | 1 | 15 |
| NRSYL | | | | | | | | | | | |
| TAVTVMAGSV | 682 | C7orf50 | chr7 | 1037310 | 1037311 | C | 7 | FS | 6 | −1 | 16 |
| PSAQSV | | | | | | | | | | | |
| FYLGYNAMQD | 683 | UBA5 | chr3 | 132394148 | 132394148 | T | 7 | FS | 8 | 1 | 15 |
| FFSYY | | | | | | | | | | | |
| NLLLKDQKPK | 684 | HIVEP1 | chr6 | 12121227 | 12121227 | A | 7 | FS | 8 | 1 | 15 |
| KTRKI | | | | | | | | | | | |
| QDTVIIKKNP | 685 | SACS | chr13 | 23912863 | 23912864 | A | 9 | FS | 8 | −1 | 15 |
| ALPKH | | | | | | | | | | | |
| AGWVGKWAGL | 686 | CLSTN3 | chr12 | 7310657 | 7310658 | C | 7 | FS | 6 | −1 | 15 |
| QLPFY | | | | | | | | | | | |
| LMLQHITLMC | 687 | GNPAT | chr1 | 231406646 | 231406649 | CTC | 2 | FS | 1 | −2 | 15 |
| SAYRN | | | | | | | | | | | |
| KKKRKIIEFR | 688 | MBD4 | chr3 | 129155547 | 129155547 | A | 10 | FS | 11 | 1 | 15 |
| IKFLF | | | | | | | | | | | |
| FLDIHNIHVM | 689 | MTM1 | chrX | 149818283 | 149818283 | A | 7 | FS | 8 | 1 | 17 |
| RESLKKS | | | | | | | | | | | |
| LRVLVLMNSK | 690 | PPP4R3B | chr2 | 55800797 | 55800800 | AGA | 2 | FS | 1 | −2 | 15 |
| HTFLA | | | | | | | | | | | |
| ECIEVYGYHN | 691 | MICU2 | chr13 | 22069321 | 22069322 | T | 6 | FS | 5 | −1 | 15 |
| IRVYK | | | | | | | | | | | |

| | | | | | | | | | | Predicted | Eli- | Eli- |
|---|---|---|---|---|---|---|---|---|---|---|---|---|
| SEQ | | | Immuno- | | SEQ | | Binding | Tumor | Sample | in the | spot | spot |
| ID | | | genicity | Wildtype | ID | HLA | Affinity | Abundance | Recur- | Discovery | test- | reac- |
| NO | HGVSc | HGVSp | Score | sequence | NO | Allele | (nM) | (TPM) | rence | Set | ed | tive |
| 592 | NM_001318120.1: | NP_001305049.1: | 1 | DQLQQAVQSQGFINYC | 789 | HLA- | 79.61 | 113.4902951 | 2 | Yes | No | NA |
| | c.1384del | p.Ile462Leufs | | QKKIDASQTEFEKNVW | | DRB1*04:04 | | | | | | |
| | | Ter16 | | SFLKVNFE | | | | | | | | |
| 593 | NM_014895.2: | NP_055710.2: | 4 | PLKMNPNILSQDSQHV | 1188 | HLA- | 101.64 | 142.8962183 | 1 | No | No | NA |
| | c.1218del | p.Phe406Leufs | | NLFFDKNDENVILQKT | | DRB1*04:04 | | | | | | |
| | | Ter8 | | TNESMENS | | | | | | | | |
| 594 | NM_001146274.1: | NP_001139746.1: | 7 | ALFGLDRQTLWCKPCR | 788 | HLA- | 101.49 | 1476.901717 | 1 | No | No | NA |
| | c.1403del | p.Lys468Serfs | | RKKKCVRYIQGEGSCL | | DRB1*01:02 | | | | | | |
| | | Ter23 | | SPPSSDGS | | | | | | | | |
| 595 | NM_003318.4: | NP_003309.2: | 8 | HYSGGESHNSSSSKTF | 1017 | HLA- | 42.84 | 75.69082705 | 3 | No | No | NA |
| | c.2560del | p.Arg854Glyfs | | EKKRGKK | | DRB1*03:01 | | | | | | |
| | | Ter39 | | | | | | | | | | |
| 596 | NM_017763.4: | NP_060233.3: | 10 | NLEPGFISIVKLESPR | 1189 | HLA- | 30.18 | 145.0109385 | 1 | Yes | No | NA |
| | c.349del | p.Arg117Alafs | | RAPRPCLSLASKARMA | | DRB1*15:01 | | | | | | |
| | | Ter41 | | GERGASAV | | | | | | | | |
| 597 | NM_024831.6: | NP_079107.6: | 22 | VHDASTSSDSEEQDMS | 1023 | HLA- | 9.31 | 96.32369965 | 1 | No | No | NA |
| | c.1674del | p.Gly559Valfs | | VKKGDDLLETNNPEPE | | DRB1*04:04 | | | | | | |
| | | Ter35 | | KCQSVSSA | | | | | | | | |
| 598 | NM_015120.4: | NP_055935.4: | 68 | KKRFKSLEKSHKNTGE | 1019 | HLA- | 99.23 | 244.2464458 | 1 | No | No | NA |
| | c.11080del | p.Ser3696Alafs | | LKKSKVLSHHRAGRSN | | DRB1*04:04 | | | | | | |
| | | Ter27 | | QIKIEQIK | | | | | | | | |
| 599 | NM_194314.2: | NP_919290.2: | 101 | CDECGKTFIRHDHLTK | 1020 | HLA- | 42.88 | 117.8015983 | 1 | No | No | NA |
| | c.1870del | p.Ile624Tyrfs | | HKKIHSGEKAHQCEEC | | DRB1*01:02 | | | | | | |
| | | Ter88 | | GKCFGRRD | | | | | | | | |
| 600 | NM_173829.3: | NP_776190.1: | 118 | QKYQKKEKKKEKKSKS | 1069 | HLA- | 23.65 | 8.043683467 | 1 | No | No | NA |
| | c.381del | p.Lys129Asnfs | | KKGKHHKKEKKKRKKE | | DRB1*04:04 | | | | | | |
| | | Ter29 | | KHSSTPNS | | | | | | | | |
| 601 | NM_015630.3: | NP_056445.3: | 141 | SEHHLQRAISAQQVFR | 1018 | HLA- | 153.22 | 90.76268639 | 1 | No | No | NA |
| | c.207del | p.Glu70Argfs | | EKKESMVIPVPEAESN | | DRB1*04:04 | | | | | | |
| | | Ter39 | | VNYYNRLY | | | | | | | | |
| 602 | NM_019589.2: | NP_062535.2: | 143 | YRTSMFKTFKKTLDDG | 1038 | HLA- | 105.62 | 34.88988455 | 1 | No | No | NA |
| | c.5778del | p.Phe1928Serfs | | FFPFIILDAINDRVRH | | DRB1*04:04 | | | | | | |
| | | Ter33 | | FDQFWSAA | | | | | | | | |
| 603 | NM_145199.2: | NP_660200.1: | 164 | GTVYHDMGNINLTFFT | 1190 | HLA- | 78.61 | 38.76618055 | 1 | Yes | No | NA |
| | c.368del | p.Lys123Serfs | | TKKKYDRMENLKLIVR | | DRB1*04:04 | | | | | | |
| | | Ter8 | | ALNAVQPQ | | | | | | | | |
| 604 | NM_002439.4: | NP_002430.3: | 198 | STSYLLCISENKENVR | 830 | HLA- | 120.22 | 53.93262102 | 2 | Yes | No | NA |
| | c.1148del | p.Lys383Argfs | | DKKKGNIFIGIVGVQP | | DRB1*04:04 | | | | | | |
| | | Ter32 | | ATGEVVFD | | | | | | | | |
| 605 | NM_001012662.2: | NP_001012680.1: | 207 | DPNFGSKEDFDSLLQS | 1046 | HLA- | 120.54 | 18.29583114 | 1 | Yes | No | NA |
| | c.902del | p.Lys301Argfs | | AKKKSIRVILDLTPNY | | DRB1*15:01 | | | | | | |
| | | Ter31 | | RGENSWFS | | | | | | | | |
| 606 | NM_152279.3: | NP_689492.3: | 228 | IHTGEKPYECSDCGKS | 1059 | HLA- | 64.98 | 26.87688773 | 2 | No | No | NA |
| | c.2106del | p.Lys702Asnfs | | FTKKSQLQVHQRIHTG | | DRB1*01:02 | | | | | | |
| | | Ter32 | | EKPYVCAE | | | | | | | | |
| 607 | NM_001317078.1: | NP_001304007.1: | 229 | MKITNGRHGDSAGAEG | 1191 | HLA- | 17.96 | 1.382080259 | 1 | No | No | NA |
| | c.36del | p.Ala14Argfs | | TMENFTALFGAQAD | | DRB1*04:10 | | | | | | |
| | | Ter72 | | PPPP | | | | | | | | |
| 608 | NM_001146694.1: | NP_001140166.1: | 231 | HVSQAQQETYLGFWIN | 1085 | HLA- | 107.14 | 25.68193545 | 2 | Yes | No | NA |
| | c.3110del | p.Ser1037Thrfs | | SKKSQCNIFLSGTY | | DRB1*04:04 | | | | | | |
| | | Ter37 | | | | | | | | | | |
| 609 | NM_014494.2: | NP_055309.2: | 285 | DPKPALRWGDSKGSNC | 1043 | HLA- | 17.59 | 12.55742051 | 1 | No | No | NA |
| | c.2409del | p.Trp804Glyfs | | QGGWEDDSAATGMVKS | | DRB1*01:01 | | | | | | |
| | | Ter99 | | NQWGNCKE | | | | | | | | |
| 610 | NM_001164342.2: | NP_001157814.1: | 324 | HKTLLERHVALHSASN | 951 | HLA- | 68.89 | 15.82316283 | 2 | Yes | No | NA |
| | c.2075del | p.Pro692Leufs | | GTPPAGTPPGARAGPP | | DRB1*04:04 | | | | | | |
| | | Ter43 | | GVVACTEG | | | | | | | | |
| 611 | NM_001145794.1: | NP_001139266.1: | 330 | WWFWPLCCKVVIKDPP | 1057 | HLA- | 99.32 | 16.01766224 | 1 | Yes | No | NA |
| | c.1069del | p.Ala357Profs | | PPPAPAPKEEEEEPLP | | DRB1*04:04 | | | | | | |
| | | Ter52 | | TKKWPTVD | | | | | | | | |
| 612 | NM_001204.6: | NP_001195.2: | 341 | KNISSEHSMSSTPLTI | 879 | HLA- | 246.82 | 177.84929 | 2 | Yes | No | NA |
| | c.1748del | p.Asn583Thrfs | | GEKNRNSINYERQQAQ | | DRB1*04:04 | | | | | | |
| | | Ter44 | | ARIPSPET | | | | | | | | |
| 613 | NM_001080517.2: | NP_001073986.1: | 402 | NYKVDCACHKGNRNCP | 1032 | HLA- | 54.54 | 117.8533251 | 2 | No | No | NA |
| | c.1246del | p.Arg416Glyfs | | IQKRNPNATELPLLPP | | DRB1*08:04 | | | | | | |
| | | Ter34 | | PPSLPTIG | | | | | | | | |
| 614 | NM_017943.3: | NP_060413.2: | 407 | DQPSILNSCEDPVPGM | 1021 | HLA- | 190.47 | 23.76940308 | 1 | No | No | NA |
| | c.1454dup | p.Leu485Phefs | | LFFLPPGQHLSDYSQL | | DRB1*04:04 | | | | | | |
| | | Ter15 | | NESTTKES | | | | | | | | |
| 615 | NM_017619.3: | NP_060089.1: | 446 | KEQDRVHSPCPTSGSE | 1025 | HLA- | 104.33 | 3.364743162 | 1 | No | No | NA |
| | c.358del | p.Arg 120Glyfs | | KKKRSDDPVEDDKEKK | | DRB1*01:02 | | | | | | |
| | | Ter18 | | ELGYLTVE | | | | | | | | |
| 616 | NM_006738.5: | NP_006729.4: | 447 | LKEQLHQKDQKILLLL | 1192 | HLA- | 151.25 | 541.8271176 | 1 | No | No | NA |
| | c.7150_7152del | p.Glu2384del | | EEKEMIFRDMAECSTP | | DRB1*15:01 | | | | | | |
| | | | | LPEDCSPT | | | | | | | | |
| 617 | NM_015062.3: | NP_055877.3: | 461 | ASSSSSSSSSSSRSRS | 1041 | HLA- | 38.69 | 13.85913686 | 1 | Yes | No | NA |
| | c.4375del | p.Ser1459Profs | | RSLSPPHKRWRRSSCS | | DRB1*04:04 | | | | | | |
| | | Ter81 | | SSGRSRRC | | | | | | | | |
| 618 | NM_000051.3: | NP_000042.3: | 467 | ELSPLLMILSQLLPQQ | 1050 | HLA- | 49.12 | 5.88459648 | 1 | No | No | NA |
| | c.1348dup | p.Glu450Glyfs | | RHGERTPYVLRCLTEV | | DRB1*13:04 | | | | | | |
| | | Ter37 | | ALCQDKRS | | | | | | | | |
| 619 | NM_001199252.1: | NP_001186181.1: | 482 | RRKSKRMSKYKENKSE | 1022 | HLA- | 158.07 | 30.61283787 | 1 | No | No | NA |
| | c.955del | p.Thr319Leufs | | NKKTVPQKKMHKSVSS | | DRB1*04:04 | | | | | | |
| | | Ter34 | | NDAYNFNL | | | | | | | | |
| 620 | NM_144682.5: | NP_653283.3: | 534 | DSLKNVIARAISKLPI | 1106 | HLA- | 65.69 | 4.067830853 | 1 | No | No | NA |
| | c.856dup | p.Cys286Leufs | | VHFCSSKPRVEYSTKI | | DRB1*04:04 | | | | | | |
| | | Ter30 | | VEVFCGKE | | | | | | | | |
| 621 | NM_053043.2: | NP_444271.2: | 558 | PPRQPFLPGPGQPFLP | 1092 | HLA- | 14.72 | 1.676333054 | 1 | No | No | NA |
| | c.1723_1724dup | p.Gln575Hisfs | | THTQPNLQGPLHPPLP | | DRB1*04:04 | | | | | | |
| | | Ter76 | | PPHQPQPQ | | | | | | | | |
| 622 | NM_004212.3: | NP_004203.2: | 560 | SILYYLGLVQWVVQKV | 1047 | HLA- | 21 | 2.268710501 | 1 | No | No | NA |
| | c.875del | p.Leu292Tyrfs | | AWFLQITMGTTATETL | | DRB1*01:02 | | | | | | |
| | | Ter23 | | AVAGNIFV | | | | | | | | |
| 623 | NM_023034.1: | NP_075447.1: | 621 | RFQELKAQRESKEALE | 1193 | HLA- | 186.9 | 13.63782303 | 1 | No | No | NA |
| | c.3197dup | p.Asn1066Lysfs | | IEKNSRKPPPYKHIKA | | DRB1*04:04 | | | | | | |
| | | Ter14 | | NKVIGKVQ | | | | | | | | |
| 624 | NM_001288718.1: | NP_001275647.1: | 636 | KPQIKQVVPEFVNASA | 1044 | HLA- | 82.04 | 5.537065595 | 2 | No | No | NA |
| | c.2144del | p.Gly715Alafs | | DAGGSSATYMDQAPSP | | DRB1*01:02 | | | | | | |
| | | Ter101 | | AVCPQAPY | | | | | | | | |
| 625 | NM_001330564.1: | NP_001317493.1: | 651 | ELVEMCNGKNGILEDS | 1027 | HLA- | 115.12 | 12.49563374 | 1 | No | No | NA |
| | c.3177dup | p.Glu1060Argfs | | QKKEDTAFSDWSDEDV | | DRB1*03:01 | | | | | | |
| | | Ter7 | | PDRTEVTE | | | | | | | | |
| 626 | NM_030771.1: | NP_110398.1: | 663 | KEYLQEKAKEKYQEWL | 1194 | HLA- | 225.75 | 18.5208182 | 1 | No | No | NA |
| | c.731del | p.Asn244Metfs | | KKKNAEECERKKKEKE | | DRB1*04:04 | | | | | | |
| | | Ter28 | | KEKQQQAE | | | | | | | | |
| 627 | NM_014680.3: | NP_055495.2: | 701 | KWCQRKLQAELKIGSF | 1045 | HLA- | 27.14 | 69.33014247 | 1 | No | No | NA |
| | c.151del | p. Trp51Glyfs | | RFFWIQNVSLKFQQHQ | | DRB1*15:01 | | | | | | |
| | | Ter77 | | QTVEIDNL | | | | | | | | |
| 628 | NM_001321699.1: | NP_001308628.1: | 707 | TAKGGVGKLVTLRNVS | 1195 | HLA- | 41.1 | 473.2706382 | 1 | No | No | NA |
| | c.805del | p.Ile269Tyrfs | | TKKIPTVNRITPKTQG | | DRB1*15:01 | | | | | | |
| | | Ter4 | | TNQIQKNT | | | | | | | | |
| 629 | NM_017763.4: | NP_060233.3: | 728 | FNLQKSSLSARHPQRK | 784 | HLA- | 187.79 | 105.9103245 | 4 | Yes | No | NA |
| | c.1976del | p.Gly659Valfs | | RRGGPSEPTPGSRPQD | | DRB1*15:01 | | | | | | |
| | | Ter41 | | ATVHPACQ | | | | | | | | |
| 630 | NM_138995.4: | NP_620482.3: | 793 | LSPVDCIPEENNSAHP | 1063 | HLA- | 22.47 | 0.086419346 | 1 | No | No | NA |
| | c.3988del | p.Ser1330Leufs | | SFFSSSSKGDSFAQH | | DRB1*03:01 | | | | | | |
| | | Ter102 | | | | | | | | | | |
| 631 | NM_001078645.2: | NP_001072113.1: | 847 | IKDKLKCYDFDVHTMK | 1058 | HLA- | 17.33 | 5.570944675 | 1 | No | No | NA |
| | c.1670del | p.Asn557Thrfs | | TLKNIISPPWDFREFE | | DRB1*01:02 | | | | | | |
| | | Ter14 | | VEKQTAEE | | | | | | | | |
| 632 | NM_001136005.1: | NP_001129477.1: | 855 | GSVLKNGSLTNHFSFE | 1196 | HLA- | 95.55 | 1.793248073 | 1 | Yes | No | NA |
| | c.2420dup | p.Ala808Glyfs | | KKKARVAVLISGTGSN | | DRB1*04:04 | | | | | | |
| | | Ter26 | | LQALIDST | | | | | | | | |
| 633 | NM_001330315.1: | NP_001317244.1: | 1007 | LKTVGKLTATQVAKIS | 1094 | HLA- | 116.99 | 4.256775848 | 1 | No | No | NA |
| | c.742del | p.Cys248Alafs | | FFFCFVWFLANLSYQE | | DRB1*08:04 | | | | | | |
| | | Ter22 | | ALSDTQVA | | | | | | | | |
| 634 | NM_016377.3: | NP_057461.2: | 1148 | KRSQENEWVKSDQVKK | 1197 | HLA- | 94.72 | 4.000178708 | 1 | No | No | NA |
| | c.236del | p.Lys79Argfs | | RKKKRKDYQPNYFLSI | | DRB1*03:01 | | | | | | |
| | | Ter21 | | PITNKEII | | | | | | | | |
| 635 | NM_016546.2: | NP_057630.2: | 1151 | ESHNFSGDIALLELQH | 1062 | HLA- | 28.48 | 11.30657953 | 1 | No | No | NA |
| | c.1051del | p.Leu351Trpfs | | SIPLGPNVLPVCLPDN | | DRB1*07:01 | | | | | | |
| | | Ter35 | | ETLYRSGL | | | | | | | | |
| 636 | NM_001277285.1: | NP_001264214.1: | 1167 | PGGLEGRLQATGQARP | 1198 | HLA- | 107.49 | 0.493888385 | 1 | No | No | NA |
| | c.2875dup | p.Arg959Profs | | PAPRPFHHGQYYGYLS | | DRB1*04:04 | | | | | | |
| | | Ter80 | | SSSPGEVE | | | | | | | | |
| 637 | NM_001135146.1: | NP_001128618.1: | 1168 | NGHIHFDNVSVVSLQD | 1101 | HLA- | 46.75 | 0.711403012 | 1 | No | No | NA |
| | c.852dup | p.Glu285Argfs | | GKKEPSSCTCLKGPKL | | DRB1*04:04 | | | | | | |
| | | Ter59 | | SEIGTIAW | | | | | | | | |
| 638 | NM_001245002.1: | NP_001231931.1: | 1189 | LSAQMLAPPPPGLPRL | 1071 | HLA- | 257.68 | 234.3696439 | 1 | No | No | NA |
| | c.1367del | p.Pro456Leufs | | ALPPATKPATTSEGGA | | DRB1*08:01 | | | | | | |
| | | Ter36 | | TSPTSPSY | | | | | | | | |
| 639 | NM_017763.4: | NP_060233.3: | 1206 | IRQHPGHAHYHLPAAY | 1087 | HLA- | 27.16 | 4.044168867 | 1 | No | No | NA |
| | c.1082del | p.Pro361Leufs | | LLGPSRSAVARPPRPG | | DRB1*01:01 | | | | | | |
| | | Ter58 | | PFLPSQEP | | | | | | | | |
| 640 | NM_024534.5: | NP_078810.1: | 1244 | WWLTGSNLTLSVNNSG | 1199 | HLA- | 129.84 | 1.413244379 | 1 | No | No | NA |
| | c.899dup | p.Leu300Phefs | | LFFLCGNGVYKGFPPK | | DRB1*04:04 | | | | | | |
| | | Ter13 | | WSGRCGLG | | | | | | | | |
| 641 | NM_006015.4: | NP_006006.3: | 1267 | PNLMPSNPDSGMYSPS | 1074 | HLA- | 5.07 | 23.15821122 | 1 | No | No | NA |
| | c.3977del | p.Pro1326Argfs | | RYPPQQQQQQQQRHDS | | DRB1*07:01 | | | | | | |
| | | Ter155 | | YGNQFSTQ | | | | | | | | |
| 642 | NM_001127500.2: | NP_001120972.1: | 1341 | SLNRITDIGEVSQFLT | 1040 | HLA- | 191.18 | 58.26789883 | 1 | No | No | NA |
| | c.34443446del | p.Ile1148del | | EGIIMKDFSHPNVLSL | | DRB1*15:01 | | | | | | |
| | | | | LGICLRSE | | | | | | | | |
| 643 | NM_182914.2: | NP_878918.2: | 1376 | WRKLVSKTQLEMNLPL | 1060 | HLA- | 42.91 | 266.371014 | 1 | No | No | NA |
| | c.2031del | p.Lys677Asnfs | | MIKKQDQPTFDNSGNI | | DRB1*15:01 | | | | | | |
| | | Ter30 | | LSKEEKAT | | | | | | | | |
| 644 | NM_024896.2: | NP_079172.2: | 1397 | MILSSYFINFIYLAKS | 1200 | HLA- | 56.24 | 11.3357601 | 1 | Yes | No | NA |
| | c.1951del | p.Thr651Profs | | TKKTMLTLTLVCAITF | | DRB1*04:04 | | | | | | |
| | | Ter3 | | LLVCSGTF | | | | | | | | |
| 645 | NM_000465.2: | NP_000456.2: | 1427 | KVRYVVSKASVQTQPA | 1201 | HLA- | 56.16 | 56.02073863 | 1 | No | No | NA |
| | c.513del | p.Asp172Metfs | | IKKDASAQQDSYEFVS | | DRB1*15:01 | | | | | | |
| | | Ter40 | | PSPPADVS | | | | | | | | |
| 646 | NM_001256798.1: | NP_001243727.1: | 1524 | DGLRSRVKYGVKTTPE | 912 | HLA- | 190.16 | 1.526850439 | 1 | Yes | No | NA |
| | c.1128dup | p.Tyr377Leufs | | SPPYSSGSYDSIKTEV | | DRB1*03:01 | | | | | | |
| | | Ter18 | | SGCPEDLT | | | | | | | | |
| 647 | NM_001172690.1: | NP_001166161.1: | 1544 | KTFRRSSHLTAHQSIH | 1202 | HLA- | 76.78 | 3.993223122 | 1 | No | No | NA |
| | c.1740del | p.Lys580Asnfs | | ADKKPYECKECGKAFK | | DRB1*08:04 | | | | | | |
| | | Ter84 | | MYGYLTQH | | | | | | | | |
| 648 | NM_001040108.1: | NP_001035197.1: | 1560 | FATTLWGVHSAQTEKE | 1203 | HLA- | 228.7 | 45.52954185 | 1 | No | No | NA |
| | c.1755del | p.Glu586Asnfs | | KKKESSNCGRRNVFSY | | DQA1*04:01/ | | | | | | |
| | | Ter24 | | GRVKLCST | | DQB1*04:02 | | | | | | |
| 649 | NM_172251.2: | NP_758455.1: | 1564 | KPDAEYPEWLFEMNLG | 1055 | HLA- | 133.38 | 0.803405824 | 1 | No | No | NA |
| | c.311dup | p.Thr106Aspfs | | PPKTLEELDPESREYW | | DQA1*05:01/ | | | | | | |
| | | Ter42 | | RRLRKQNI | | DQB1*02:01 | | | | | | |
| 650 | NM_005667.3: | NP_005658.1: | 1580 | LAGGRHCCPVCRWPSY | 1029 | HLA- | 244.93 | 4.998220761 | 1 | No | No | NA |
| | c.2009dup | p.Gln671Alafs | | KKKQPYAQHQPLSNDV | | DRB1*04:04 | | | | | | |
| | | Ter12 | | PS | | | | | | | | |
| 651 | NM_198557.2: | NP_940959.1: | 1630 | SVFGKEVTLETLVKDL | 1204 | HLA- | 110.88 | 3.382487536 | 1 | Yes | No | NA |
| | c.406del | p.Ile136Serfs | | KKKIPSLSFSPLKPNG | | DRB1*03:01 | | | | | | |
| | | Ter4 | | RISVEGSF | | | | | | | | |
| 652 | NM_017744.4: | NP_060214.2: | 1652 | ETADRELLPTFHHVSV | 1205 | HLA- | 161.24 | 1.850059667 | 1 | No | No | NA |
| | c.1520del | p.Lys507Argfs | | YPKKELPLFIHFTAGF | | DRB1*15:01 | | | | | | |
| | | Ter19 | | CSSTAMIA | | | | | | | | |
| 653 | NM_001010870.2: | NP_001010870.1: | 1678 | ASINKKLGLLSYKDRI | 1206 | HLA- | 51.19 | 1.875783751 | 1 | No | No | NA |
| | c.3504del | p.Glu1169Lysfs | | RKKESEVLCSTTETLE | | DRB1*07:01 | | | | | | |
| | | Ter19 | | EKNENMKL | | | | | | | | |
| 654 | NM_004360.3: | NP_004351.1: | 1720 | PDEIGNFIDENLKAAD | 1035 | HLA- | 221.04 | 9.292722995 | 1 | No | No | NA |
| | c.2466del | p.Thr823Glnfs | | TDPTAPPYDSLLVFDY | | DRB1*15:01 | | | | | | |
| | | Ter23 | | EGSGSEAA | | | | | | | | |
| 655 | NM_005788.3: | NP_005779.1: | 1797 | TISLVAVSDVNKHADR | 1093 | HLA- | 27.42 | 22.74415789 | 1 | No | No | NA |
| | c.1147del | p.Trp383Glyfs | | IAFWDDVYGFKMSCMK | | DRB1*01:01 | | | | | | |
| | | Ter12 | | KAVIPEAV | | | | | | | | |
| 656 | NM_194463.1: | NP_919445.1: | 1823 | VIEVGKKHGPWVNHYS | 1034 | HLA- | 233.26 | 0.648212697 | 1 | No | No | NA |
| | c.629dup | p.Val211Argfs | | IFFVSVSFFIITAATV | | DRB1*04:04 | | | | | | |
| | | Ter42 | | GYFIFYSA | | | | | | | | |
| 657 | NM_001310338.1: | NP_001297267.1: | 1850 | EKYSNLVQSVLSSRGV | 1117 | HLA- | 122.63 | 19.27029849 | 2 | No | No | NA |
| | c.206del | p.Pro69Argfs | | AQTPGSVEEDALLCGP | | DRB1*01:02 | | | | | | |
| | | Ter14 | | VSKHKLPN | | | | | | | | |
| 658 | NM_053275.3: | NP_444505.1: | 1857 | LALSVETDYTFPLAEK | 1068 | HLA- | 107.27 | 2.141099432 | 1 | No | No | NA |
| | c.804_806del | p.Phe268del | | VKAFLADPSAFVAAAP | | DRB1*04:04 | | | | | | |
| | | | | VAAATTAA | | | | | | | | |
| 659 | NM_000213.4: | NP_000204.3: | 1896 | VLVHKKKDCPPGSFWW | 1073 | HLA- | 311.52 | 44.63939167 | 2 | No | No | NA |
| | c.2149del | p.Leu717Cysfs | | LIPLLLLLLPLLALLL | | DRB1*01:02 | | | | | | |
| | | Ter52 | | LLCWKYCA | | | | | | | | |
| 660 | NM_022470.3: | NP_071915.1: | 1927 | LCNVTLNSAQQAQAHY | 1075 | HLA- | 143.99 | 0.567026138 | 1 | No | No | NA |
| | c.278del | p.Asn93Ilefs | | QGKNHGKKLRNYYAAN | | DRB1*04:04 | | | | | | |
| | | Ter21 | | SCPPPARM | | | | | | | | |
| 661 | NM_001162429.2: | NP_001155901.1: | 1999 | DTVGTLSLIMLAQAQE | 1207 | HLA- | 129.23 | 2.252648934 | 1 | No | No | NA |
| | c.602dup | p.Leu201Phefs | | VFFLKATRDKMKDAII | | DRB1*04:04 | | | | | | |
| | | Ter7 | | AKLANQAA | | | | | | | | |
| 662 | NM_019589.2: | NP_062535.2: | 2010 | LPTMPPPVLPPSLPPP | 1107 | HLA- | 24.09 | 74.31176528 | 1 | No | No | NA |
| | c.1646del | p.Pro549Leufs | | VMPPALPATVPPPGMP | | DRB1*15:01 | | | | | | |
| | | Ter98 | | PPVMPPSL | | | | | | | | |
| 663 | NM_015412.3: | NP_056227.2: | 2025 | SFTQLSEEIQMAVVWC | 1091 | HLA- | 61.14 | 12.3569003 | 1 | No | No | NA |
| | c.1155del | p.Lys385Asnfs | | RSKKLKAQAIFLGNKL | | DRB1*15:01 | | | | | | |
| | | Ter33 | | LKSNRLKH | | | | | | | | |
| 664 | NM_018025.2: | NP_060495.2: | 2033 | LDDLITPAKLSVGFEL | 1208 | HLA- | 258.78 | 2.106070591 | 1 | No | No | NA |
| | c.487dup | p.Met163Asnfs | | LRKMGWKEGQGVGPRV | | DRB1*03:01 | | | | | | |
| | | Ter23 | | KRRPRRQK | | | | | | | | |
| 665 | NM_0046962: | NP_004687.1: | 2079 | NGSFYFSGICYLLSSV | 1033 | HLA- | 44.74 | 7.365770553 | 1 | No | No | NA |
| | c.1425del | p.Phe475Leufs | | SFFFVPLAERWKNSLT | | DRB1*04:04 | | | | | | |
| | | Ter12 | | | | | | | | | | |
| 666 | NM_015208.4: | NP_056023.3: | 2129 | KHMSLSYVANQEPGIL | 1088 | HLA- | 112.11 | 38.36741167 | 1 | Yes | No | NA |
| | c.4577del | p.Asn1526Metfs | | QQKNAVQIISSALDTD | | DRB1*15:01 | | | | | | |
| | | Ter10 | | NESTKDTE | | | | | | | | |
| 667 | NM_014856.2: | NP_055671.2: | 2203 | PDEVCYRVLMQLCSHY | 1209 | HLA- | 240.68 | 1.560387719 | 1 | No | No | NA |
| | c.2488del | p.Val830Cysfs | | GQPVLSVRVMLEMRQA | | DRB1*04:04 | | | | | | |
| | | Ter62 | | GIVPNTIT | | | | | | | | |
| 668 | NM_017943.3: | NP_060413.2: | 2213 | QMVAFLEQRASALLAS | 1056 | HLA- | 316.78 | 18.7306847 | 1 | No | No | NA |
| | c.683dup | p.Asn228Lysfs | | CSKNCTNSPAIVRESG | | DRB1*04:04 | | | | | | |
| | | Ter23 | | QSRGVPAV | | | | | | | | |
| 669 | NM_001164665.1: | NP_001158137.1: | 2237 | YLESSLISHESAVTAL | 1079 | HLA- | 126.92 | 0.685593482 | 1 | No | No | NA |
| | c.23152316dup | p.Gly773Profs | | VPPGSESFDILTAGIQ | | DRB1*04:04 | | | | | | |
| | | Ter9 | | ATSPLTTV | | | | | | | | |
| 670 | NM_020722.1: | NP_065773.1: | 2270 | AIARLDNSAAKHKLAV | 1065 | HLA- | 239.4 | 18.50746611 | 1 | No | No | NA |
| | c.501del | p.Lys167Asnfs | | KPKKQRVSKKHRRLAQ | | DRB1*04:04 | | | | | | |
| | | Ter82 | | DPQHEQGG | | | | | | | | |
| 671 | NM_001321378.1: | NP_001308307.1: | 2328 | RQRELQLSVLSAESLR | 1061 | HLA- | 33.07 | 3.403730165 | 1 | No | No | NA |
| | c.4970del | p.Phe1657Serfs | | ENFFLGGVTLPLKDFN | | DRB1*01:01 | | | | | | |
| | | Ter5 | | LSKETVKW | | | | | | | | |
| 672 | NM_001033925.1: | NP_001029097.1: | 2329 | TEDIKSAFAPFGKISD | 1210 | HLA- | 9.15 | 1.541755407 | 1 | No | No | NA |
| | c.436439del | p.Val146Lysfs | | ARVVKDMATGKSKGYG | | DRB1*01:01 | | | | | | |
| | | Ter62 | | FVSFYNKL | | | | | | | | |
| 673 | NM_001077242.1: | NP_001070710.1: | 2341 | SIINTLQTQVEVKKRR | 1211 | HLA- | 97.91 | 5.378949565 | 1 | No | No | NA |
| | c.180del | p.Lys60Asnfs | | HRLKRHNDCFVGSEAV | | DRB1*04:04 | | | | | | |
| | | Ter20 | | DVIFSHLI | | | | | | | | |
| 674 | NM_001130475.1: | NP_001123947.1: | 2359 | VPTIFSLPEDNQGKDP | 1026 | HLA- | 342.13 | 43.93258991 | 1 | No | No | NA |
| | c.297del | p.Lys99Asnfs | | SKKKSQKKNLEDEKEV | | DRB1*08:01 | | | | | | |
| | | Ter25 | | CPKAKSEE | | | | | | | | |
| 675 | NM_001270.2: | NP_001261.2: | 2365 | LQTRADYLIKLLSRDL | 1082 | HLA- | 32.95 | 33.14096218 | 1 | No | No | NA |
| | c.3960del | p.Glu1321Lysfs | | AKKEALSGAGSSKRRK | | DRB1*01:02 | | | | | | |
| | | Ter22 | | ARAKKNKA | | | | | | | | |
| 676 | NM_000038.5: | NP_000029.2: | 2424 | SDNFNTGNMTVLSPYL | 1212 | HLA- | 191.81 | 151.270049 | 1 | No | No | NA |
| | c.2489del | p.Val830Glyfs | | NTTVLPSSSSSRGSLD | | DQA1*05:05/ | | | | | | |
| | | Ter12 | | SSRSEKDR | | DQB1*03:19 | | | | | | |
| 677 | NM_015446.4: | NP_056261.4: | 2685 | SDLSSQFVISPPALRS | 1077 | HLA- | 156.86 | 5.324297496 | 1 | No | No | NA |
| | c.6518del | p.Asn2173Thrfs | | RQKNTSNKNKLEDELK | | DRB1*08:04 | | | | | | |
| | | Ter12 | | DDAQSVET | | | | | | | | |
| 678 | NM_003482.3: | NP_003473.3: | 2725 | SLLHTAGGGSHGQLGS | 1213 | HLA- | 69.52 | 34.38722995 | 1 | No | No | NA |
| | c.12373_12375del | p.Ser4125del | | GSSSEASSVPHLLAQP | | DQA1*05:05/ | | | | | | |
| | | | | SVSLGDQP | | DQB1*03:19 | | | | | | |
| 679 | NM_023073.3: | NP_075561.3: | 2729 | ILTSLWLLEQPYFATY | 1024 | HLA- | 315.66 | 169.4167381 | 1 | No | No | NA |
| | c.5408del | p.Asn1803Metfs | | KAKNAIIKMVENRDTG | | DRB1*04:04 | | | | | | |
| | | Ter7 | | CQIGPNIE | | | | | | | | |
| 680 | NM_003594.3: | NP_003585.3: | 2732 | QLHHLKLSEDEETVYN | 1214 | HLA- | 33.55 | 1.977505051 | 1 | No | No | NA |
| | c.2577dup | p.Ala860Cysfs | | VFFARSRSALQSYLKR | | DRB1*04:04 | | | | | | |
| | | Ter15 | | HESRGNQS | | | | | | | | |
| 681 | NM_002740.5: | NP_002731.4: | 2737 | LRVIGRGSYAKVLLVR | 1215 | HLA- | 125.2 | 1.097724896 | 1 | No | No | NA |
| | c.826dup | p.Thr276Asnfs | | LKKTDRIYAMKVVKKE | | DRB1*04:04 | | | | | | |
| | | Ter16 | | LVNDDEDI | | | | | | | | |
| 682 | NM_001318252.1: | NP_001305181.1: | 2744 | VQKAEALMRELDEEGS | 1076 | HLA- | 193.88 | 0.153634394 | 2 | No | No | NA |
| | c.535del | p.Leu179Cysfs | | DPPLPGRAQRIRQVLQ | | DQA1*05:01/ | | | | | | |
| | | Ter136 | | LLS | | DQB1*03:02 | | | | | | |
| 683 | NM_024818.3: | NP_079094.1: | 2769 | LNFGTVSFYLGYNAMQ | 1072 | HLA- | 110.93 | 1.455171443 | 1 | No | No | NA |
| | c.876dup | p.Pro293Serfs | | DFFPTMSMKPNPQCDD | | DRB1*04:04 | | | | | | |
| | | Ter12 | | RNCRKQQE | | | | | | | | |
| 684 | NM_002114.2: | NP_002105.2: | 2770 | NQSVEQMCNLLLKDQK | 1216 | HLA- | 303.42 | 12.762296 | 1 | No | No | NA |
| | c.1206dup | p.Gln403Thrfs | | PKKQGKYICEYCNRAC | | DRB1*03:01 | | | | | | |
| | | Ter7 | | AKPSVLLK | | | | | | | | |
| 685 | NM_014363.5: | NP_055178.3: | 2771 | LKIEETNPSLAQDTVI | 1064 | HLA- | 289.02 | 12.01526875 | 1 | No | No | NA |
| | c.5151del | p.Lys1717Asnfs | | IKKKSCSSKALNTPVL | | DRB1*01:02 | | | | | | |
| | | Ter8 | | SVLKEAAK | | | | | | | | |
| 686 | NM_014718.3: | NP_055533.2: | 2774 | DSEVADSPSSDERRII | 1108 | HLA- | 88.41 | 45.25526815 | 1 | Yes | No | NA |
| | c.2858del | p.Pro953Hisfs | | ETPPHRY | | DRB1*15:01 | | | | | | |
| | | Ter106 | | | | | | | | | | |
| 687 | NM_014236.3: | NP_055051.1: | 2776 | VDSGDSEVVDGLMLQH | 1217 | HLA- | 38.74 | 2.267287363 | 1 | No | No | NA |
| | c.1426_1428del | p.Leu476del | | ITLLMCSAYRNQLLNI | | DRB1*01:01 | | | | | | |
| | | | | FVRPSLVA | | | | | | | | |
| 688 | NM_003925.2: | NP_003916.1: | 2787 | ACGETLSVTSEENSLV | 834 | HLA- | 325.4 | 3.950365507 | 1 | No | No | NA |
| | c.939dup | p.Glu314Argfs | | KKKERSLSSGSNFCSE | | DRB1*04:04 | | | | | | |
| | | Ter13 | | QKTSGIIN | | | | | | | | |
| 689 | NM_000252.2: | NP_000243.1: | 2837 | ELFFLDIHNIHVMRES | 1218 | HLA- | 188.64 | 1.927864858 | 1 | No | No | NA |
| | c.969dup | p.Val324Serfs | | LKKVKDIVYPNVEESH | | DRB1*04:04 | | | | | | |
| | | Ter8 | | WLSSLEST | | | | | | | | |
| 690 | NM_001122964.1: | NP_001116436.1: | 2849 | VEHHTYHIKNYIMNKD | 1122 | HLA- | 55.57 | 5.084704108 | 2 | No | No | NA |
| | c.17201722del | p.Arg574del | | LLRRVLVLMNSKHTFL | | DRB1*08:04 | | | | | | |
| | | | | ALCALRFM | | | | | | | | |
| 691 | NM_152726.2: | NP_689939.1: | 2915 | FDLDGDECLSHEEFLG | 1219 | HLA- | 11.7 | 3.130971359 | 1 | No | No | NA |
| | c.1178del | p.Asn393Thrfs | | VLKNRMHRGLWVPQHQ | | DRB1*15:01 | | | | | | |
| | | Ter22 | | SIQEYWKC | | | | | | | | |

SUPPLEMENTARY TABLE 11
List of the Top 100 most recurrent predicted MHC-II neoAgs, with immunogenic score, obtained from
the computational methods in the validation set.

| | | | | | | | Reference | | Altered | Number | Pep- |
|---|---|---|---|---|---|---|---|---|---|---|---|
| Mutant | | | | | | Micro- | MS | | MS | deleted | tide |
| Epitope | SEQ | Gene | Chromo- | | | satellite | lengths | Variant | Length | nucleo- | Len- |
| Sequence | ID NO | Name | some | Start | Stop | motif | (repeats) | Type | (repeats) | tides | gth |
| CFFFFCYILNT | 692 | CNOT1 | chr16 | 58577316 | 58577328 | A | 13 | FS | 12 | −1 | 15 |
| MFDR | | | | | | | | | | | |
| RMQLCTQLARF | 629 | RNF43 | chr17 | 56435160 | 56435161 | G | 7 | FS | 6 | −1 | 15 |
| FPIT | | | | | | | | | | | |
| APLRLWSWCGT | 693 | BCORL1 | chrX | 129190010 | 129190011 | C | 7 | FS | 6 | −1 | 15 |
| SQTY | | | | | | | | | | | |
| PKKKRSAFPSR | 379 | MARCKS | chr6 | 114181210 | 114181220 | A | 11 | FS | 10 | −1 | 15 |
| SLSS | | | | | | | | | | | |
| TRLMAPVGSVM | 694 | XYLT2 | chr17 | 48433967 | 48433973 | C | 7 | FS | 6 | −1 | 15 |
| SCSL | | | | | | | | | | | |
| FFFSVIFSTRC | 695 | CNOT1 | chr16 | 58577316 | 58577328 | A | 13 | FS | 11 | −1 | 15 |
| LTDS | | | | | | | | | | | |
| QLFVMSDTTYK | 595 | TTK | chr6 | 80751896 | 80751897 | A | 9 | FS | 8 | −1 | 15 |
| IYWT | | | | | | | | | | | |
| KKRKIIEFRIK | 696 | MBD4 | chr3 | 129155547 | 129155547 | A | 10 | FS | 11 | 1 | 14 |
| FLF | | | | | | | | | | | |
| QVPALAPQAWW | 697 | ZBTB20 | chr3 | 114058002 | 114058003 | G | 7 | FS | 6 | −1 | 15 |
| PARR | | | | | | | | | | | |
| EFLSHPFAVTL | 698 | LRP1 | chr12 | 57572241 | 57572241 | G | 7 | FS | 8 | 1 | 15 |
| YGGG | | | | | | | | | | | |
| MSVCFFFLLYS | 699 | CNOT1 | chr16 | 58577316 | 58577328 | A | 13 | FS | 12 | −2 | 15 |
| QHDV | | | | | | | | | | | |
| PSQVWTAATLR | 700 | DOCK3 | chr3 | 51417604 | 51417610 | C | 7 | FS | 6 | −1 | 15 |
| CPAV | | | | | | | | | | | |
| KEALFLQEVFQ | 336 | MARCKS | chr6 | 114181210 | 114181220 | A | 11 | FS | 12 | 1 | 15 |
| AERL | | | | | | | | | | | |
| RGRMLTWRSSL | 701 | MXRA8 | chr1 | 1290109 | 1290110 | C | 7 | FS | 6 | −1 | 15 |
| WLQG | | | | | | | | | | | |
| PQSLRPVRVRA | 702 | TSC22D4 | chr7 | 100075074 | 100075075 | G | 6 | FS | 5 | −1 | 15 |
| HPGL | | | | | | | | | | | |
| RSKFTGLCRPL | 703 | TCF7 | chr5 | 133451702 | 133451703 | C | 5 | FS | 4 | −1 | 15 |
| TSLA | | | | | | | | | | | |
| RSSTTAGLPAR | 704 | CUEDC1 | chr17 | 55962834 | 55962835 | C | 6 | FS | 5 | −1 | 15 |
| CAAW | | | | | | | | | | | |
| RGEAMGSGQAT | 705 | TMEM132D | chr12 | 130184704 | 130184705 | C | 7 | FS | 6 | −1 | 16 |
| VTSMS | | | | | | | | | | | |
| NHIVVSAEGNI | 706 | GLTSCR1L | chr6 | 42832626 | 42832627 | A | 7 | FS | 6 | −1 | 15 |
| SKKQ | | | | | | | | | | | |
| IPPLHLRTSAR | 707 | TCF7 | chr5 | 133473764 | 133473765 | C | 7 | FS | 6 | −1 | 15 |
| SKFT | | | | | | | | | | | |
| GEAMGSGQATV | 708 | TMEM132D | chr12 | 130184704 | 130184704 | C | 6 | FS | 8 | 1 | 16 |
| TSMSP | | | | | | | | | | | |
| TNMEIPHFFVI | 709 | OR7C2 | chr19 | 15052828 | 15052829 | T | 7 | FS | 6 | −1 | 15 |
| LPKS | | | | | | | | | | | |
| CQKKLMLLRLN | 592 | SEC31A | chr4 | 83785564 | 83785565 | T | 9 | FS | 8 | −1 | 15 |
| LRKM | | | | | | | | | | | |
| QARLCLIVSRT | 604 | MSH3 | chr5 | 79970914 | 79970915 | A | 8 | FS | 7 | −1 | 15 |
| LLLV | | | | | | | | | | | |
| RSLTCVLSVGR | 606 | ZNF585B | chr19 | 37676332 | 37676333 | A | 6 | FS | 5 | −1 | 15 |
| PLAT | | | | | | | | | | | |
| GKLMHVLYFSS | 608 | KDM4C | chr9 | 7170005 | 7170006 | A | 7 | FS | ins G | −1 | 15 |
| NEVT | | | | | | | | | | | |
| KTEIQLTMNDS | 612 | BMPR2 | chr2 | 203420129 | 203420130 | A | 7 | FS | 6 | −1 | 15 |
| KHKL | | | | | | | | | | | |
| QKGILMLQNCH | 613 | SETD5 | chr3 | 9486784 | 9486785 | A | 6 | FS | 5 | −1 | 15 |
| SYHL | | | | | | | | | | | |
| MYSIRMENSTW | 624 | STAT5A | chr17 | 40461419 | 40461420 | G | 5 | FS | 4 | −1 | 15 |
| MRPW | | | | | | | | | | | |
| LVQSVLSSRGV | 657 | MGME1 | chr20 | 17950704 | 17950705 | G | 4 | FS | 3 | −1 | 15 |
| AQTR | | | | | | | | | | | |
| AWHFSRAATEV | 659 | ITGB4 | chr17 | 73733649 | 73733650 | C | 5 | FS | 4 | −1 | 15 |
| TWWA | | | | | | | | | | | |
| TAVTVMAGSVP | 682 | C7orf50 | chr7 | 1037310 | 1037311 | C | 7 | FS | 6 | −1 | 16 |
| SAQSV | | | | | | | | | | | |
| LRVLVLMNSKH | 690 | PPP4R3B | chr2 | 55800797 | 55800800 | AGA | 2 | FS | 1 | −2 | 15 |
| TFLA | | | | | | | | | | | |
| KKKTYTCAITT | 710 | SEC63 | chr6 | 108214773 | 108214773 | A | 9 | FS | 10 | 1 | 15 |
| VKAT | | | | | | | | | | | |
| NIDLCTALSAL | 711 | USP15 | chr12 | 62783241 | 62783244 | NA | NA | FS | NA | −2 | 15 |
| SGIP | | | | | | | | | | | |
| VPQLLHLPQFH | 712 | KMT2B | chr19 | 36211898 | 36211899 | C | 7 | FS | 6 | −1 | 15 |
| SLRR | | | | | | | | | | | |
| SSLITMLTPRQ | 713 | ITGA6 | chr2 | 173330355 | 173330356 | G | 5 | FS | 4 | −1 | 15 |
| KARK | | | | | | | | | | | |
| LOGFIQDRAGR | 714 | BAX | chr19 | 49458970 | 49458971 | G | 8 | FS | 7 | −1 | 15 |
| MGGR | | | | | | | | | | | |
| REKIINPTISC | 715 | AKAP7 | chr6 | 131481275 | 131481276 | A | 8 | FS | 7 | −1 | 15 |
| PFQS | | | | | | | | | | | |
| VGTMSSSWRLW | 716 | SH3BGRL3 | chr1 | 26607374 | 26607375 | C | 4 | FS | 3 | −1 | 15 |
| NKTR | | | | | | | | | | | |
| QTPASLMITRA | 717 | WRAP53 | chr17 | 7606714 | 7606714 | G | 6 | FS | 8 | 1 | 15 |
| RKGR | | | | | | | | | | | |
| SPLPRGSGAAP | 718 | TEAD3 | chr6 | 35446236 | 35446237 | C | 4 | FS | 3 | −1 | 16 |
| LSWDS | | | | | | | | | | | |
| KTYTCAITTVK | 719 | SEC63 | chr6 | 108214773 | 108214775 | T | 9 | FS | 7 | −1 | 16 |
| ATETK | | | | | | | | | | | |
| QLLDLKSSLLK | 720 | CRYBG3 | chr3 | 97593629 | 97593630 | A | 5 | FS | 4 | −1 | 15 |
| RPIH | | | | | | | | | | | |
| FRQDKLKVMRK | 721 | ZBTB7A | chr19 | 4053969 | 4053972 | NA | NA | FS | NA | −2 | 15 |
| HTG | | | | | | | | | | | |
| VQSLLMYKDGD | 722 | DENND1C | chr19 | 6468935 | 6468936 | G | 7 | FS | 6 | −1 | 18 |
| SVLQRGA | | | | | | | | | | | |
| IKGSESATYVP | 723 | LARP1 | chr5 | 154173389 | 154173389 | C | 7 | FS | 8 | 1 | 16 |
| VAPPH | | | | | | | | | | | |
| EETQEKMTILQ | 724 | ATP6V1G1 | chr9 | 117359886 | 117359889 | NA | NA | FS | NA | −2 | 15 |
| TYFR | | | | | | | | | | | |
| MLGNVESGGPH | 725 | BCL9 | chr1 | 147094075 | 147094076 | C | 6 | FS | 5 | −1 | 16 |
| LLQPA | | | | | | | | | | | |
| ENALLNGSSFL | 726 | SPECC1L | chr22 | 24718455 | 24718456 | NA | NA | FS | NA | −1 | 16 |
| VSSSI | | | | | | | | | | | |
| RPFFLPVYRQT | 727 | CCDC168 | chr13 | 103381996 | 103382004 | T | 9 | FS | 8 | −1 | 15 |
| HWRL | | | | | | | | | | | |
| MCTWLTMAPSC | 728 | TGM6 | chr20 | 2384126 | 2384131 | C | 6 | FS | 5 | −1 | 15 |
| LRRS | | | | | | | | | | | |
| TLPVQRLRALS | 729 | GATA3 | chr10 | 8100728 | 8100734 | C | 7 | FS | 8 | 1 | 15 |
| QNER | | | | | | | | | | | |
| PPAALRSGIPR | 730 | CARMIL2 | chr16 | 67682131 | 67682136 | C | 6 | FS | 7 | 1 | 15 |
| LLHQ | | | | | | | | | | | |
| SGWRRLHRAMP | 731 | SPTBN5 | chr15 | 42145897 | 42145902 | G | 6 | FS | 5 | −1 | 15 |
| SSGA | | | | | | | | | | | |
| PPPLQRLQGHL | 732 | KCNH4 | chr17 | 40328259 | 40328265 | G | 7 | FS | 8 | 1 | 15 |
| GRPY | | | | | | | | | | | |
| QAPFFCLRVRC | 733 | ABCC5 | chr3 | 183665257 | 183665265 | A | 9 | FS | 8 | −1 | 15 |
| GGWL | | | | | | | | | | | |
| VWMASASVTAS | 734 | TNR | chr1 | 175372615 | 175372620 | G | 6 | FS | 5 | −1 | 16 |
| TAGMT | | | | | | | | | | | |
| LVAISFKTVFK | 735 | AVPR1A | chr12 | 63541343 | 63541348 | T | 6 | FS | 5 | −1 | 15 |
| ASHA | | | | | | | | | | | |
| NVTFLMQATLC | 736 | PKHD1 | chr6 | 51890015 | 51890021 | T | 7 | FS | 6 | −1 | 15 |
| ARQE | | | | | | | | | | | |
| ASITSISRTTS | 737 | PRELP | chr1 | 203452549 | 203452554 | C | 6 | FS | 5 | −1 | 15 |
| SLSS | | | | | | | | | | | |
| QVIVSRGGALS | 738 | GLYR1 | chr16 | 4862229 | 4862236 | C | 8 | FS | 9 | 1 | 16 |
| GSPRL | | | | | | | | | | | |
| GVQGPSMATVA | 739 | TMEM143 | chr19 | 48866745 | 48866750 | C | 6 | FS | 7 | 1 | 16 |
| RAPRA | | | | | | | | | | | |
| ESTPTASSAMA | 740 | ENTPD2 | chr9 | 139945517 | 139945522 | C | 6 | FS | 5 | −1 | 16 |
| VTRSS | | | | | | | | | | | |
| TTLWLGPASVA | 741 | ZDHH | chr22 | 20130522 | 20130527 | C | 6 | FS | 5 | −1 | 16 |
| PATLP | | C8 | | | | | | | | | |
| SDGTPGGAPAQ | 742 | ARAF | chrX | 47426415 | 47426420 | C | 6 | FS | 5 | −1 | 16 |
| PACPR | | | | | | | | | | | |
| RLQGLAGAPRG | 743 | MEFV | chr16 | 3304412 | 3304417 | G | 6 | FS | 5 | −1 | 15 |
| RRSA | | | | | | | | | | | |
| GPELRRSRAPR | 744 | TBX2 | chr17 | 59482061 | 59482066 | C | 6 | FS | 5 | −1 | 15 |
| TATR | | | | | | | | | | | |
| PSTAVATWRRC | 745 | TULP4 | chr6 | 158923337 | 158923342 | C | 6 | FS | 5 | −1 | 15 |
| AGPA | | | | | | | | | | | |
| AMMNGKVPFFS | 746 | C22orf24 | chr22 | 32334105 | 32334113 | A | 9 | FS | 8 | −1 | 15 |
| ALKV | | | | | | | | | | | |
| PLTSGGSAAAT | 747 | LARP6 | chr15 | 71125203 | 71125204 | T | 5 | FS | 4 | −1 | 15 |
| AKWG | | | | | | | | | | | |
| HTGTQFFEIKS | 748 | VASH1 | chr14 | 77237566 | 77237569 | AGA | 2 | FS | 1 | −2 | 15 |
| RPLT | | | | | | | | | | | |
| LGQILILPPWA | 749 | PRR14L | chr22 | 32099548 | 32099549 | C | 6 | FS | 5 | −1 | 15 |
| FLDP | | | | | | | | | | | |
| RSWRFLRAAPS | 750 | THEMIS2 | chr1 | 28208611 | 28208612 | C | 5 | FS | 4 | −1 | 15 |
| SSAR | | | | | | | | | | | |
| GPQLLLSHRAV | 751 | ATG4D | chr19 | 10662979 | 10662981 | CT | 2 | FS | 1 | −1 | 15 |
| PHVH | | | | | | | | | | | |
| TNLRVQLLKRQ | 752 | NTN4 | chr12 | 96131808 | 96131810 | NA | NA | FS | NA | −1 | 15 |
| LSLS | | | | | | | | | | | |
| QGEVDSQQGAR | 753 | CEP250 | chr20 | 34061365 | 34061367 | TC | 3 | FS | 2 | −1 | 15 |
| AAAE | | | | | | | | | | | |
| AILLQVIAKKM | 754 | TDRD15 | chr2 | 21362231 | 21362232 | A | 7 | FS | 6 | −1 | 15 |
| TSTL | | | | | | | | | | | |
| LEQLFLCALKA | 755 | BEST4 | chr] | 45250035 | 45250036 | C | 4 | FS | 3 | −1 | 15 |
| RAFR | | | | | | | | | | | |
| QERVRLIPRLR | 756 | BARHL1 | chr9 | 135464864 | 135464865 | C | 7 | FS | 6 | −1 | 15 |
| SLPR | | | | | | | | | | | |
| SPSTPGAATAA | 757 | APCDD1 | chr18 | 10485691 | 10485692 | C | 4 | FS | 3 | −1 | 16 |
| ASSRP | | | | | | | | | | | |
| RTFCLTARRGA | 758 | PRX | chr19 | 40900434 | 40900436 | TC | 2 | FS | 1 | −1 | 15 |
| LAIR | | | | | | | | | | | |
| EEVVCLLFPAS | 759 | COL4A2 | chr13 | 111156324 | 111156330 | C | 7 | FS | 6 | −1 | 15 |
| GEMK | | | | | | | | | | | |
| RRRATLSAAFA | 760 | GZF1 | chr20 | 23345851 | 23345852 | A | 5 | FS | 4 | −1 | 15 |
| RRRF | | | | | | | | | | | |
| STLPRPFRTRM | 761 | MAGEE2 | chrX | 75004753 | 75004754 | C | 6 | FS | 5 | −1 | 15 |
| TWRS | | | | | | | | | | | |
| PMFLALDRRGG | 762 | FGF22 | chr19 | 643527 | 643528 | G | 8 | FS | 7 | −1 | 15 |
| PGQA | | | | | | | | | | | |
| PTCLLLSRPLR | 763 | YLPM1 | chr14 | 75230470 | 75230471 | G | 6 | FS | 5 | −1 | 15 |
| SPHL | | | | | | | | | | | |
| LREWTTQAPLR | 764 | SCN10A | chr3 | 38766674 | 38766675 | NA | NA | FS | NA | −1 | 15 |
| AARW | | | | | | | | | | | |
| FLPWVPERGVA | 765 | C14orf80 | chr14 | 105964215 | 105964216 | G | 5 | FS | 4 | −1 | 15 |
| SWTW | | | | | | | | | | | |
| VQMRSRMSSSA | 766 | IFFO2 | chr1 | 19235144 | 19235145 | C | 5 | FS | 4 | −1 | 15 |
| RRMW | | | | | | | | | | | |
| GPLVPGLVLGG | 767 | DDX51 | chr12 | 132628264 | 132628270 | G | 7 | FS | 8 | 1 | 16 |
| VREEE | | | | | | | | | | | |
| LPALSILQRSP | 768 | ZFR2 | chr19 | 3831692 | 3831693 | C | 6 | FS | 5 | −1 | 15 |
| RLPR | | | | | | | | | | | |
| AQSSWRSLEAS | 769 | SPG7 | chr16 | 89598370 | 89598371 | C | 7 | FS | 6 | −1 | 15 |
| ALPV | | | | | | | | | | | |
| PESFKRQARAR | 770 | FDX1L | chr19 | 10426563 | 10426564 | G | 11 | FS | 10 | −1 | 15 |
| LERR | | | | | | | | | | | |
| SPVTQITGAAA | 771 | PNPLA7 | chr9 | 140414400 | 140414401 | A | 5 | FS | 4 | −1 | 15 |
| RQLL | | | | | | | | | | | |
| YAGGVGAQLM | 772 | SRRT | chr7 | 100479331 | 100479332 | G | 14 | FS | 13 | −1 | 15 |
| APLSP | | | | | | | | | | | |

| | | | | | | | | | | | |
|---|---|---|---|---|---|---|---|---|---|---|---|
| VTELAQVIVSR | 773 | GLYR1 | chr16 | 4862229 | 4862236 | G | 8 | FS | 10 | 1 | 15 |
| GGGA | | | | | | | | | | | |
| PTHVRAAARVS | 774 | FIZ1 | chr19 | 56109216 | 56109217 | C | 4 | FS | 3 | −1 | 15 |
| STAS | | | | | | | | | | | |
| RRPCCWMGAAA | 775 | GJA3 | chr13 | 20716371 | 20716372 | C | 5 | FS | 4 | −1 | 15 |
| VWRG | | | | | | | | | | | |
| HDLGLHVLSCR | 776 | PAPPA | chr9 | 119097161 | 119097173 | NA | NA | FS | NA | −11 | 15 |
| IIPV | | | | | | | | | | | |

| | | | | | | | | | | Predicted | Eli- | |
|---|---|---|---|---|---|---|---|---|---|---|---|---|
| SEQ | | | Immuno- | | SEQ | | Binding | Tumor | Sample | in the | spot | re- |
| ID | | | genicity | Wildtype | ID | HLA | Affinity | Abundance | Recur- | Discovery | test- | ac- |
| NO | HGVSc | HGVSp | Score | sequence | NO | Allele | (nM) | (TPM) | rence | Set | ed | tive |
| 692 | NM_206999.2: | NP_996882.1: | 17493 | ILDCNSVRQSIMSVCF | 1109 | HLA- | 117.44 | 0 | 6 | No | No | NA |
| | c.4628del | p.Leu1544Cysfs | | FFFLLYSQHDV | | DRB1*04:10 | | | | | | |
| | | Ter11 | | | | | | | | | | |
| 629 | NM_017763.4: | NP_060233.3: | 728 | FNLQKSSLSARHPQRK | 784 | HLA- | 187.79 | 105.910325 | 4 | Yes | No | NA |
| | c.1976del | p.Gly659Valfs | | RRGGPSEPTPGSRPQD | | DRB1*15:01 | | | | | | |
| | | Ter41 | | ATVHPACQ | | | | | | | | |
| 693 | NM_001184772.2: | NP_001171701.1: | 10739 | SSQLLTPAERPGGLDD | 898 | HLA- | 858.42 | 111.813323 | 4 | Yes | No | NA |
| | c.5264del | p.Pro1755Glnfs | | RSPPGSSETVELVRYE | | DRB1*01:02 | | | | | | |
| | | Ter20 | | PDLLRLLG | | | | | | | | |
| 379 | NM_002356.5: | NP_002347.5: | 18560 | PKAEDGATPSPSNETP | 888 | HLA- | 135.1 | 0 | 4 | Yes | No | NA |
| | c.464del | p.Lys155Argfs | | KKKKKRFSFKKSFKLS | | DRB1*01:01 | | | | | | |
| | | Ter12 | | GFSFKKNK | | | | | | | | |
| 694 | NM_022167.2: | NP_071450.2: | 19163 | VNQEVLEILDFHLYGS | 859 | HLA- | 48.56 | 0 | 4 | Yes | No | NA |
| | c.1584del | p.Gly529Alafs | | YPPGTPALKAYWENT | | DRB1*01:01 | | | | | | |
| | | Ter78 | | YDAADGPSG | | | | | | | | |
| 695 | NM_206999.2: | NP_996882.1: | 19690 | IILDCNSVRQSIMSVC | 1110 | HLA- | 343.44 | 0 | 4 | No | No | NA |
| | c.4627_4628del | p.Phe1543Serfs | | FFFFLLYSQHDV | | DRB1*04:04 | | | | | | |
| | | Ter22 | | | | | | | | | | |
| 595 | NM_003318.4: | NP_003309.2: | 8 | HYSGGESHNSSSSKTF | 1017 | HLA- | 42.84 | 75.6908271 | 3 | No | No | NA |
| | c.2560del | p.Arg854Glyfs | | EKKRGKK | | DRB1*03:01 | | | | | | |
| | | Ter39 | | | | | | | | | | |
| 696 | NM_003925.2: | NP_003916.1: | 9889 | ACGETLSVTSEENSLV | 834 | HLA- | 722.91 | 3.95036551 | 3 | No | No | NA |
| | c.939dup | p.Glu314Argfs | | KKKERSLSSGSNFCSE | | DRB1*04:04 | | | | | | |
| | | Ter13 | | QKTSGIIN | | | | | | | | |
| 697 | NM_001164342.2: | NP_001157814.1: | 11670 | HKTLLERHVALHSASN | 951 | HLA- | 822.45 | 15.8231628 | 3 | Yes | No | NA |
| | c.2075del | p.Pro692Leufs | | GTPPAGTPPGARAGPP | | DRB1*04:04 | | | | | | |
| | | Ter43 | | GVVACTEG | | | | | | | | |
| 698 | NM_002332.2: | NP_002323.2: | 11876 | VLRGHEFLSHPFAVTL | 1220 | HLA- | 621.05 | 9.35558875 | 3 | No | No | NA |
| | c.4468dup | p.Glu1490Glyfs | | YGGEVYWTDWRTNTLA | | DRB1*01:02 | | | | | | |
| | | Ter6 | | KANKWTGH | | | | | | | | |
| 699 | NM_206999.2: | NP_996882.1: | 19617 | IILDCNSVRQSIMSVC | 1110 | HLA- | 183.48 | 0 | 3 | No | No | NA |
| | c.4626_4628del | p.Phe1543del | | FFFFLLYSQHDV | | DRB1*04:04 | | | | | | |
| 700 | NM_004947.4: | NP_004938.1: | 20560 | KGHYSLHFDAFHHPLG | 877 | HLA- | 392.31 | 0 | 3 | Yes | No | NA |
| | c.5555del | p.Pro1852Glnfs | | DTPPALPARTLRKSPL | | DRB1*04:04 | | | | | | |
| | | Ter45 | | HPIPASPT | | | | | | | | |
| 336 | NM_002356.5: | NP_002347.5: | 22504 | KAEDGATPSPSNETPK | 1221 | HLA- | 36.89 | 0 | 3 | Yes | Yes | No |
| | c.464_465insA | p.Lys156Glufs | | KKKKRFSFKKSFKLSG | | DRB1*01:01 | | | | | | |
| | | Ter28 | | FSFKKNKK | | | | | | | | |
| 701 | NM_001282585.1: | NP_001269514.1: | 23519 | HERRVFHLTVAEPHAE | 1111 | HLA- | 41.22 | 0 | 3 | Yes | No | NA |
| | c.901del | p.Arg301Glyfs | | PPPRGSPGNGSSHSGA | | DRB1*15:01 | | | | | | |
| | | Ter107 | | PGPDPTLA | | | | | | | | |
| 702 | NM_030935.3: | NP_112197.1: | 26121 | SKAKAEKPPLSASSPQ | 1222 | HLA- | 85.01 | 0 | 3 | No | No | NA |
| | c.587del | p.Pro196Glnfs | | QRPPEPETGESAGTSR | | DRB1*08:04 | | | | | | |
| | | Ter21 | | AATPLPSL | | | | | | | | |
| 703 | NM_001346425.1: | NP_001333354.1: | 32044 | SAFNLLMHYPPPSGAG | 1223 | HLA- | 22.25 | 0 | 3 | Yes | No | NA |
| | c.424del | p.Gln142Serfs | | QHPQPQPPLHKANQPP | | DRB1*11:04 | | | | | | |
| | | Ter57 | | HGVPQLSL | | | | | | | | |
| 704 | NM_001271875.1: | NP_001258804.1: | 33061 | SGGGGTAGARGGGGGT | 1168 | HLA- | 233.88 | 0 | 3 | No | No | NA |
| | c.91del | p.Gln31Argfs | | AAPQELNNSRPARQVR | | DQA1*04:01/ | | | | | | |
| | | Ter57 | | RLEFNQAM | | DQB1*03:19 | | | | | | |
| 705 | NM_133448.2: | NP_597705.2: | 33402 | DLGLCVAELELLSSWF | 901 | HLA- | 197.65 | 0 | 3 | No | No | NA |
| | c.618del | p.Thr207Argfs | | SPPTVVAGRRKSVDQP | | DQA1*04:01/ | | | | | | |
| | | Ter75 | | EGTPVELY | | DQB1*03:19 | | | | | | |
| 706 | NM_001318819.1: | NP_001305748.1: | 35854 | FHLVPNHIVVSAEGNI | 1224 | HLA- | 28.4 | 0 | 3 | No | No | NA |
| | c.2689del | p.Thr897GInfs | | SKKTECLGRALKFDKV | | DRB1*01:01 | | | | | | |
| | | Ter8 | | GLVQYQST | | | | | | | | |
| 707 | NM_001346425.1: | NP_001333354.1: | 38991 | GAGQHPQPQPPLHKAN | 1113 | HLA- | 44.38 | 0 | 3 | Yes | No | NA |
| | c.463del | p.His155Thrfs | | QPPHGVPQLSLYEHFN | | DRB1*08:04 | | | | | | |
| | | Ter44 | | SPHPTPAP | | | | | | | | |
| 708 | NM_133448.2: | NP_597705.2: | 75709 | DLGLCVAELELLSSWF | 901 | HLA- | 818.06 | 0 | 3 | No | No | NA |
| | c.617_618dup | p.Thr207Profs | | SPPTVVAGRRKSVDQP | | DQA1*05:01/ | | | | | | |
| | | Ter76 | | EGTPVELY | | DQB1*03:02 | | | | | | |
| 709 | NM_012377.1: | NP_036509.1: | 90913 | TLTILRLSFCTNMEIP | 1225 | HLA- | 766.53 | 0 | 3 | No | No | NA |
| | c.535del | p.Cys179Valfs | | HFFCDPSEVLKLACSD | | DRB1*08:04 | | | | | | |
| | | Ter7 | | TFINNIVM | | | | | | | | |
| 592 | NM_001318120.1: | NP_001305049.1: | 1 | DQLQQAVQSQGFINYC | 789 | HLA- | 79.61 | 113.490295 | 2 | Yes | No | NA |
| | c.1384del | p.Ile462Leufs | | QKKIDASQTEFEKNVW | | DRB1*04:04 | | | | | | |
| | | Ter16 | | SFLKVNFE | | | | | | | | |
| 604 | NM_002439.4: | NP_002430.3: | 198 | STSYLLCISENKENVR | 830 | HLA- | 120.22 | 53.932621 | 2 | Yes | No | NA |
| | c.1148del | p.Lys383Argfs | | DKKKGNIFIGIVGVQP | | DRB1*04:04 | | | | | | |
| | | Ter32 | | ATGEVVFD | | | | | | | | |
| 606 | NM_152279.3: | NP_689492.3: | 228 | IHTGEKPYECSDCGKS | 1059 | HLA- | 64.98 | 26.8768877 | 2 | No | No | NA |
| | c.2106del | p.Lys702Asnfs | | FTKKSQLQVHQRIHTG | | DRB1*01:02 | | | | | | |
| | | Ter32 | | EKPYVCAE | | | | | | | | |
| 608 | NM_001146694.1: | NP_001140166.1: | 231 | HVSQAQQETYLGFWIN | 1085 | HLA- | 107.14 | 25.6819355 | 2 | Yes | No | NA |
| | c.3110del | p.Ser1037Thrfs | | SKKSQCNIFLSGTY | | DRB1*04:04 | | | | | | |
| | | Ter37 | | | | | | | | | | |
| 612 | NM_001204.6: | NP_001195.2: | 341 | KNISSEHSMSSTPLTI | 878 | HLA- | 246.82 | 177.84929 | 2 | Yes | No | NA |
| | c.1748del | p.Asn583Thrfs | | GEKNRNSINYERQQAQ | | DRB1*04:04 | | | | | | |
| | | Ter44 | | ARIPSPET | | | | | | | | |
| 613 | NM_001080517.2: | NP_001073986.1: | 402 | NYKVDCACHKGNRNCP | 1032 | HLA- | 54.54 | 117.853325 | 2 | No | No | NA |
| | c.1246del | p.Arg416Glyfs | | IQKRNPNATELPLLPP | | DRB1*08:04 | | | | | | |
| | | Ter34 | | PPSLPTIG | | | | | | | | |
| 624 | NM_001288718.1: | NP_001275647.1: | 636 | KPQIKQVVPEFVNASA | 1044 | HLA- | 82.04 | 5.5370656 | 2 | No | No | NA |
| | c.2144del | p.Gly715Alafs | | DAGGSSATYMDQAPSP | | DRB1*01:02 | | | | | | |
| | | Ter101 | | AVCPQAPY | | | | | | | | |
| 657 | NM_001310338.1: | NP_001297267.1: | 1850 | EKYSNLVQSVLSSRGV | 1117 | HLA- | 122.63 | 19.2702985 | 2 | No | No | NA |
| | c.206del | p.Pro69Argfs | | AQTPGSVEEDALLCGP | | DRB1*01:02 | | | | | | |
| | | Ter14 | | VSKHKLPN | | | | | | | | |
| 659 | NM_000213.4: | NP_000204.3: | 1896 | VLVHKKKDCPPGSFWW | 1073 | HLA- | 311.52 | 44.6393917 | 2 | No | No | NA |
| | c.2149del | p.Leu717Cysfs | | LIPLLLLLLPLLALLL | | DRB1*01:02 | | | | | | |
| | | Ter52 | | LLCWKYCA | | | | | | | | |
| 682 | NM_001318252.1: | NP_001305181.1: | 2744 | VQKAEALMRELDEEGS | 1076 | HLA- | 193.88 | 0.15363439 | 2 | No | No | NA |
| | c.535del | p.Leu179Cysfs | | DPPLPGRAQRIRQVLQ | | DQA1*05:01/ | | | | | | |
| | | Ter136 | | LLS | | DQB1*03:02 | | | | | | |
| 690 | NM_001122964.1: | NP_001116436.1: | 2849 | VEHHTYHIKNYIMNKD | 1122 | HLA- | 55.57 | 5.08470411 | 2 | No | No | NA |
| | c.1720_1722del | p.Arg574del | | LLRRVLVLMNSKHTFL | | DRB1*08:04 | | | | | | |
| | | | | ALCALRFM | | | | | | | | |
| 710 | NM_007214.4: | NP_009145.1: | 2942 | GGWQQKSKGPKKTAKS | 1119 | HLA- | 243.34 | 0.68754272 | 2 | No | No | NA |
| | c.1586dup | p.Lys530Glufs | | KKKKPLKKKPTPVLLP | | DRB1*04:04 | | | | | | |
| | | Ter30 | | QSKQQKQK | | | | | | | | |
| 711 | NM_001252078.1: | NP_001239007.1: | 3734 | DPLTKPMQYKVVVPKI | 1226 | HLA- | 292.6 | 34.6079337 | 2 | No | No | NA |
| | c.1507_1509del | p.Leu503del | | GNILDLCTALSALSGI | | DRB1*08:04 | | | | | | |
| | | | | PADKMIVT | | | | | | | | |
| 712 | NM_014727.1: | NP_055542.1: | 4042 | ARSSRVIKTPRRFMDE | 1099 | HLA- | 114.95 | 9.7861849 | 2 | No | No | NA |
| | c.1656del | p.Lys553Asnfs | | DPPKPPKVEVSPVLRP | | DRB1*15:01 | | | | | | |
| | | Ter52 | | PITTSPPV | | | | | | | | |
| 713 | NM_001079818.1: | NP_001073286.1: | 4242 | LQRANRTGGLYSCDIT | 1121 | HLA- | 22.49 | 1.32218459 | 2 | No | No | NA |
| | c.276del | p.Pro93Hisfs | | ARGPCTRIEFDNDADP | | DRB1*08:04 | | | | | | |
| | | Ter37 | | TSESKEDQ | | | | | | | | |
| 714 | NM_001291428.1: | NP_001278357.1: | 4860 | TGALLLQGFIQDRAGR | 904 | HLA- | 270.69 | 2.768255100 | 2 | No | No | NA |
| | c.121del | p.Glu41Argfs | | MGGEAPELALDPVPQD | | DRB1*01:02 | | | | | | |
| | | Ter19 | | ASTKKLSE | | | | | | | | |
| 715 | NM_016377.3: | NP_057461.2: | 5005 | KRSQENEWVKSDQVKK | 1227 | HLA- | 502.02 | 4.00017871 | 2 | No | No | NA |
| | c.236del | p.Lys79Argfs | | RKKKRKDYQPNYFLSI | | DRB1*04:04 | | | | | | |
| | | Ter21 | | PITNKEII | | | | | | | | |
| 716 | NM_031286.3: | NP_112576.1: | 5130 | DISQDNALRDEMRALA | 1114 | HLA- | 255.11 | 8.75580723 | 2 | No | No | NA |
| | c.171del | p.Lys58Argfs | | GNPKATPPQIVNGDQY | | DRB1*08:04 | | | | | | |
| | | Ter33 | | CGDYELFV | | | | | | | | |
| 717 | NM_001143990.1: | NP_001137462.1: | 5339 | LSTRHVHLECRLQLWW | 1228 | HLA- | 189.23 | 0.08469557 | 2 | Yes | No | NA |
| | c.1563_1564dup | p.Ala522Glyfs | | CGGAPDSSIPDDHQGE | | DRB1*04:04 | | | | | | |
| | | Ter27 | | KGQGGTEG | | | | | | | | |
| 718 | NM_003214.3: | NP_003205.2: | 6685 | QIVSASVLQNKFSPPS | 1120 | HLA- | 188.93 | 1.40943535 | 2 | No | No | NA |
| | c.454del | p.Gln152Argfs | | PLPQAVFSTSSRFWSS | | DQA1*04:01/ | | | | | | |
| | | Ter115 | | PPLLGQQP | | DQB1*03:19 | | | | | | |
| 719 | NM_007214.4: | NP_009145.1: | 7135 | KGGWQQKSKGPKKTAK | 1104 | HLA- | 322.18 | 0.87823747 | 2 | No | No | NA |
| | c.1585_1586del | p.Lys529Glufs | | SKKKKPLKKKPTPVLL | | DQA1*01:02/ | | | | | | |
| | | Ter30 | | PQSKQQKQ | | DQB1*06:02 | | | | | | |
| 720 | NM_153605.3: | NP_705833.3: | 8663 | EARRRAHDQLLDLKSS | 1229 | HLA- | 199.6 | 4.9165498 | 2 | No | No | NA |
| | c.3596del | p.Lys1199Argfs | | LLKKADTLIGEIFNSV | | DRB1*08:04 | | | | | | |
| | | Ter5 | | REELKFKH | | | | | | | | |
| 721 | NM_015898.3: | NP_056982.1: | 8824 | IRTHTGEKPYECNICK | 1230 | HLA- | 503.64 | 26.6214875 | 2 | No | No | NA |
| | c.1259_1261del | p.Thr420del | | VRFTRQDKLKVHMRKH | | DRB1*08:04 | | | | | | |
| | | | | TGEKPYLC | | | | | | | | |
| 722 | NM_024898.3: | NP_079174.2: | 10932 | KGVQSLLMYKDGDSVL | 1231 | HLA- | 435.67 | 4.8082823 | 2 | No | No | NA |
| | c.1436del | p.Gly479Alafs | | QRGGSLRAPALPSRSD | | DQA1*01:01/ | | | | | | |
| | | Ter3 | | RLQQRLPI | | DQB1*05:01 | | | | | | |
| 723 | NM_015315.4: | NP_056130.2: | 11054 | NRGEIKGSESATYVPV | 1232 | HLA- | 633.37 | 3.84716807 | 2 | No | No | NA |
| | c.675dup | p.Thr226Hisfs | | APPTPAWQPEIKPEPA | | DQA1*05:01/ | | | | | | |
| | | Ter19 | | WHDQDETS | | DQB1*03:02 | | | | | | |
| 724 | NM_004888.3: | NP_004879.1: | 11574 | KAKEAAALGSRGSCST | 1118 | HLA- | 512.33 | 0.31139164 | 2 | No | No | NA |
| | c.223_225del | p.Lys75del | | EVEKETQEKMTILQTY | | DRB1*01:02 | | | | | | |
| | | | | FRQNRDEV | | | | | | | | |
| 725 | NM_004326.3: | NP_004317.2: | 11829 | APLTMASPAMLGNVES | 1233 | HLA- | 528.57 | 3.2282781 | 2 | No | No | NA |
| | c.2912del | p.Pro971Hisfs | | GGPPPPTASQPASVNI | | DQA1*04:01/ | | | | | | |
| | | Ter11 | | PGSLPSST | | DQB1*03:19 | | | | | | |
| 726 | NM_015330.4: | NP_056145.4: | 13006 | KSGRYMELEQRYMDLA | 1115 | HLA- | 551.26 | 3.43298181 | 2 | No | No | NA |
| | c.1508del | p.Arg503Leufs | | ENARFEREQLLGVQQH | | DQA1*04:01/ | | | | | | |
| | | Ter14 | | LSNTLKMA | | DQB1*03:19 | | | | | | |
| 727 | NM_001146197.1: | NP_001139669.1: | 17445 | PHHDDINFYSERKQNR | 1125 | HLA- | 107.46 | 0 | 2 | No | No | NA |
| | c.21050del | p.Phe7017Leufs | | PFFFACVPADSLEVIP | | DRB1*08:04 | | | | | | |
| | | Ter25 | | KTIRWTIP | | | | | | | | |
| 728 | NM_198994.2: | NP_945345.2: | 17650 | ARQDLGPSYNGWQVLD | 1126 | HLA- | 59.98 | 0 | 2 | Yes | No | NA |
| | c.1078del | p.Gln360Argfs | | ATPQEESEGVFRCGPA | | DRB1*01:02 | | | | | | |
| | | Ter99 | | SVTAIREG | | | | | | | | |
| 729 | NM_001002295.1: | NP_001002295.1: | 17795 | PITTYPPYVPEYSSGL | 1123 | HLA- | 125.99 | 0 | 2 | No | No | NA |
| | c.708_709insC | p.Ser237Glnfs | | FPPSSLLGGSPTGFGC | | DRB1*04:04 | | | | | | |
| | | Ter67 | | KSRPKARS | | | | | | | | |
| 730 | NM_001013838.1: | NP_001013860.1: | 18086 | RAGRGGLGPPAGVANS | 1133 | HLA- | 50.64 | 0 | 2 | No | No | NA |
| | c.1253_1254insC | p.Gln419Alafs | | LPPQLFAAVSRGCCTS | | DRB1*01:02 | | | | | | |
| | | Ter112 | | LTHLDASR | | | | | | | | |
| 731 | NM_016642.3: | NP_057726.4: | 18152 | ARLQTEACRLGQLHPA | 1141 | HLA- | 114.87 | 0 | 2 | No | No | NA |
| | c.9862del | p.Gly3288Alafs | | APGGLAKVQEAWATLQ | | DRB1*08:04 | | | | | | |
| | | Ter40 | | AKAQERGQ | | | | | | | | |
| 732 | NM_012285.2: | NP_036417.1: | 18191 | NVFEPKPSVPEYKVAS | 1155 | HLA- | 50.95 | 0 | 2 | No | No | NA |
| | c.641_642insG | p.Ser215Valfs | | VGGSRCLLLHYSVSKA | | DRB1*01:02 | | | | | | |
| | | Ter39 | | IWDGLILL | | | | | | | | |
| 733 | NM_005688.3: | NP_005679.2: | 18205 | QEFLHRYQELLDDNQA | 1234 | HLA- | 163.54 | 0 | 2 | No | No | NA |
| | c.3268del | p.Leu1090Cysfs | | PFFLFTCAMRWLAVRL | | DRB1*01:02 | | | | | | |
| | | Ter26 | | DLISIALI | | | | | | | | |
| 734 | NM_003285.2: | NP_003276.3: | 18315 | WFGKNCSEPYCPLGCS | 1128 | HLA- | 79.6 | 0 | 2 | No | No | NA |
| | c.636del | p.Val213Cysfs | | SRGVCVDGQCICDSEY | | DQA1*04:01/ | | | | | | |
| | | Ter52 | | SGDDCSEL | | DQB1*03:19 | | | | | | |
| 735 | NM_000706.4: | NP_000697.1: | 18393 | ITALLGSLNSCCNPWI | 1124 | HLA- | 154.59 | 0 | 2 | No | No | NA |
| | c.1052del | p.Phe351Leufs | | YMFFSGHLLQDCVQSF | | DRB1*04:04 | | | | | | |
| | | Ter19 | | PCCQNMKE | | | | | | | | |
| 736 | NM_138694.3: | NP_619639.3: | 18520 | TADEPMVFVDDQLPCN | 1131 | HLA- | 102.6 | 0 | 2 | No | No | NA |
| | c.4592del | p.Phe1531Leufs | | VTFFNASHVVCQTRDL | | DRB1*08:04 | | | | | | |
| | | Ter61 | | APGPHYLS | | | | | | | | |
| 737 | NM_002725.3: | NP_002716.1: | 18768 | LPPGPPSIFPDCPREC | 1127 | HLA- | 57.73 | 0 | 2 | No | No | NA |
| | c.242del | p.Pro81Leufs | | YCPPDFPSALYCDSRN | | DRB1*08:04 | | | | | | |
| | | Ter61 | | LRKVPVIP | | | | | | | | |
| 738 | NM_001324098.1: | NP_001311027.1: | 19046 | TVDADTVTELAQVIVS | 1235 | HLA- | 195.06 | 0 | 2 | No | No | NA |
| | c.1154_1155insG | p.Arg386Alafs | | RGGRFLEAPVSGNQQL | | DQA1*04:01/ | | | | | | |
| | | Ter15 | | SNDGMLVI | | DQB1*03:19 | | | | | | |
| 739 | NM_018273.2: | NP_060743.2: | 19289 | ELWLRLRGKGLAMLHV | 1176 | HLA- | 79.21 | 0 | 2 | No | No | NA |
| | c.66_67insG | p.Val23Glyfs | | TRGVWGSRVRVWPLLP | | DQA1*04:01/ | | | | | | |
| | | Ter105 | | ALLGPPRA | | DQB1*03:19 | | | | | | |
| 740 | NM_203468.2: | NP_982293.1: | 19310 | WVGRWFRPRKGTLGAM | 1130 | HLA- | 95.78 | 0 | 2 | No | No | NA |
| | c.610del | p.Gly204Valfs | | DLGGASTQITFETTSP | | DQA1*04:01/ | | | | | | |
| | | Ter171 | | AEDRASEV | | DQB1*03:19 | | | | | | |
| 741 | NM_001185024.1: | NP_001171953.1: | 19339 | RRGGDHVALQPLRSEG | 1129 | HLA- | 102.52 | 0 | 2 | No | No | NA |
| | c.1374del | p.Thr459Argfs | | GPPTPHRSIFAPHALP | | DQA1*04:01/ | | | | | | |
| | | Ter177 | | NRNGSLSY | | DQB1*03:19 | | | | | | |
| 742 | NM_001256196.1: | NP_001243125.1: | 19536 | GQSFSTDAAGSRGGSD | 1135 | HLA- | 134.08 | 0 | 2 | No | No | NA |
| | c.772del | p.Arg258Glyfs | | GTPRGSPSPASVSSGR | | DQA1*04:01/ | | | | | | |
| | | Ter37 | | KSPHSKSP | | DQB1*03:19 | | | | | | |
| 743 | NM_000243.2: | NP_000234.1: | 20291 | EVRLRRNASSAGRLQG | 1182 | HLA- | 202.1 | 0 | 2 | No | No | NA |
| | c.655del | p.Gly219Alafs | | LAGGAPGQKECRPFEV | | DRB1*08:04 | | | | | | |
| | | Ter43 | | YLPSGKMR | | | | | | | | |
| 744 | NM_005994.3: | NP_005985.3: | 21084 | CKPERDGAESDASSCD | 1164 | HLA- | 283.29 | 0 | 2 | No | No | NA |
| | c.987del | p.Ala330Argfs | | PPPAREPPTSPGAAPS | | DRB1*08:04 | | | | | | |
| | | Ter38 | | PLRLHRAR | | | | | | | | |
| 745 | NM_0202454: | NP_064630.2: | 21593 | CLKKGDFSLYPTSVHY | 1132 | HLA- | 329.04 | 0 | 2 | No | No | NA |
| | c.2647del | p.Leu883Trpfs | | QTPLGYERITTFDSSG | | DRB1*08:04 | | | | | | |
| | | Ter43 | | NVEEVCRP | | | | | | | | |
| 746 | NM_001302819.1: | NP_001289748.1: | 22642 | DFLSVKWEAAMMNGKV | 931 | HLA- | 407.8 | 0 | 2 | Yes | No | NA |
| | c.148del | p.Phe50Serfs | | PFFFSSESLGYFATGR | | DRB1*08:04 | | | | | | |
| | | Ter6 | | PADNVMTT | | | | | | | | |
| 747 | NM_018357.2: | NP_060827.2: | 23329 | TPQKNGRVQEKVMEHL | 1180 | HLA- | 62.97 | 0 | 2 | No | No | NA |
| | c.663del | p.Phe221Leufs | | LKLFGTFGVISSVRIL | | DQA1*04:01/ | | | | | | |
| | | Ter56 | | KPGRELPP | | DQB1*03:19 | | | | | | |
| 748 | NM_014909.4: | NP_055724.1: | 23707 | RYIRELQYNHTGTQFF | 1177 | HLA- | 46.32 | 0 | 2 | No | No | NA |
| | c.437_439del | p.Lys146del | | EIKKSRPLTGLMDLAK | | DRB1*01:02 | | | | | | |
| | | | | EMTKEALP | | | | | | | | |
| 749 | NM_173566.2: | NP_775837.2: | 23718 | GLDELDGVKAACPCPQ | 1171 | HLA- | 61.89 | 0 | 2 | No | No | NA |
| | c.5987del | p.Pro1996Glnfs | | SSPPEQKEAEPEKRPK | | DRB1*01:02 | | | | | | |
| | | Ter42 | | KVSQIRIR | | | | | | | | |
| 750 | NM_001105556.1: | NP_001099026.1: | 23857 | HFIKPLLLSEVLAWEG | 1139 | HLA- | 13.68 | 0 | 2 | No | No | NA |
| | c.781del | p.Leu261Cysfs | | PFPLSMEILEVPEGRP | | DRB1*01:02 | | | | | | |
| | | Ter34 | | IFLSPWVG | | | | | | | | |
| 751 | NM_032885.5: | NP_116274.3: | 23893 | DPSCTVGFYAGDRKEF | 1236 | HLA- | 50.2 | 0 | 2 | No | No | NA |
| | c.1224_1225del | p.Cys409Leufs | | ETLCSELTRVLSSSSA | | DRB1*08:04 | | | | | | |
| | | Ter60 | | TERYPMFT | | | | | | | | |
| 752 | NM_021229.3: | NP_067052.2: | 23998 | KVQEQLKITNLRVQLL | 1237 | HLA- | 23.92 | 0 | 2 | No | No | NA |
| | c.698_699del | p.Ser233Leufs | | KRQSCPCQRNDLNEEP | | DRB1*08:04 | | | | | | |
| | | Ter7 | | QHFTHYAI | | | | | | | | |
| 753 | NM_007186.5: | NP_009117.2: | 24061 | GERDTLAGQTVDLQGE | 1161 | HLA- | 126.48 | 0 | 2 | No | No | NA |
| | c.1382_1383del | p.Leu461Glnfs | | VDSLSKERELLQKARE | | DRB1*01:02 | | | | | | |
| | | Ter80 | | ELRQQLEV | | | | | | | | |
| 754 | NM_001306137.1: | NP_001293066.1: | 25091 | YFKKLVLNKAILLQVI | 1140 | HLA- | 42.25 | 0 | 2 | No | No | NA |
| | c.1899del | p.Asp634Metfs | | AKKDDKYTVNIQSVEA | | DRB1*08:04 | | | | | | |
| | | Ter6 | | SENIDVIS | | | | | | | | |
| 755 | NM_153274.2: | NP_695006.1: | 25096 | APAAQTPLLGRFLGVG | 1154 | HLA- | 52.51 | 0 | 2 | No | No | NA |
| | c.1268del | p.Pro423Argfs | | APSPAISLRNFGRVRG | | DRB1*08:04 | | | | | | |
| | | Ter97 | | TPRPPHLL | | | | | | | | |
| 756 | NM_020064.3: | NP_064448.1: | 25170 | RILIHGLQGASEPPPP | 1145 | HLA- | 10.04 | 0 | 2 | No | No | |
| | c.946del | p.Leu316Trpfs | | LPPLAGVLPRAAQPR | | DRB1*08:04 | | | | | | |
| | | Ter184 | | | | | | | | | | NA |
| 757 | NM_153000.4: | NP_694545.1: | 25569 | NNTWEGHYYHYSDPVC | 1238 | HLA- | 48.95 | 0 | 2 | No | No | NA |
| | c.1011del | p.Thr338Profs | | KHPTFSIYARGRYSRG | | DQA1*04:01/ | | | | | | |
| | | Ter29 | | VLSSRVMG | | DQB1*03:19 | | | | | | |
| 758 | NM_181882.2: | NP_870998.2: | 26377 | VGGEGAEEQPPGAERT | 1142 | HLA- | 108.71 | 0 | 2 | No | No | NA |
| | c.3823_3824del | p.Ser1275Thrfs | | FCLSLPDVELSPSGGN | | DRB1*01:02 | | | | | | |
| | | Ter49 | | HAEYQVAE | | | | | | | | |
| 759 | NM_001846.2: | NP_001837.2: | 26603 | QGRRGPPGAPGEMGPQ | 1136 | HLA- | 516.9 | 0 | 2 | No | No | NA |
| | c.4275del | p.Gly1426Glufs | | GPPGEPGFRGAPGKAG | | DRB1*08:04 | | | | | | |
| | | Ter33 | | PQGRGGVS | | | | | | | | |
| 760 | NM_001317012.1: | NP_001303941.1: | 26692 | CPQDQSPDRVGTEMEQ | 1147 | HLA- | 67.84 | 0 | 2 | No | No | NA |
| | c.836del | p.Asn279Metfs | | VSKNEGCQAGAELEEL | | DRB1*01:02 | | | | | | |
| | | Ter55 | | SKKAGPEE | | | | | | | | |
| 761 | NM_138703.4: | NP_619648.1: | 26706 | IQATNASGSPTSMLVV | 1152 | HLA- | 138.2 | 0 | 2 | Yes | No | NA |
| | c.133del | p.Gln45Serfs | | DAPQCPQAPINSQCVN | | DRB1*08:04 | | | | | | |
| | | Ter27 | | TSQAVQDP | | | | | | | | |
| 762 | NM_020637.1: | NP_065688.1: | 27356 | RWRRRGQPMFLALDRR | 1239 | HLA- | 154.69 | 0 | 2 | No | No | NA |
| | c.444del | p.Arg150Glyfs | | GGPRPGGRTRRYHLSA | | DRB1*03:01 | | | | | | |
| | | Ter? | | HFLPVLVS | | | | | | | | |
| 763 | NM_019589.2: | NP_062535.2: | 27397 | LQPHHLPPPPLPPPPV | 1158 | HLA- | 42.2 | 0 | 2 | No | No | NA |
| | c.284del | p.Gly95Alafs | | MPGGGYGDWQPPPPPM | | DRB1*08:04 | | | | | | |
| | | Ter124 | | PPPPGPAL | | | | | | | | |
| 764 | NM_006514.3: | NP_006505.3: | 27465 | SEDLAPSLGETWKDES | 1151 | HLA- | 89.65 | 0 | 2 | No | No | NA |
| | c.3218del | p.Val1073Alafs | | VPQVPAEGVDDTSSSE | | DRB1*01:02 | | | | | | |
| | | Ter20 | | GSTVDCLD | | | | | | | | |
| 765 | NM_001134875.1: | NP_00112834.1: | 27826 | EVPAAASQPTFLPWVP | 1240 | HLA- | 169.37 | 0 | 2 | No | No | NA |
| | c.857del | 7p.Gly286Valfs | | ERGGGELDLVVRELQA | | DRB1*01:02 | | | | | | |
| | | Ter7 | | LEEELREA | | | | | | | | |
| 766 | NM_001136265.1: | NP_001129737.1: | 27879 | METCRRLIKGSADRNS | 1156 | HLA- | 196.25 | 0 | 2 | No | No | NA |
| | c.1464del | p.Ser489Alafs | | PSPSSVASSDSGSTDE | | DRB1*01:02 | | | | | | |
| | | Ter32 | | IQDEFERE | | | | | | | | |
| 767 | NM_175066.3: | NP_778236.2: | 28278 | GPALEEAAGPLVPGLV | 1241 | HLA- | 442.34 | 0 | 2 | No | No | NA |
| | c.494_495insG | p.Phe166Valfs | | LGGFGKRKAPKVQPFL | | DQA1*04:01/ | | | | | | |
| | | Ter19 | | PRWLAEPN | | DQB1*03:19 | | | | | | |
| 768 | NM_015174.1: | NP_055989.1: | 28419 | PTATGVQPESSASIVT | 1148 | HLA- | 70.33 | 0 | 2 | No | No | NA |
| | c.563del | p.Pro188Argfs | | SYPPPSYNPTCTAYTA | | DRB1*08:04 | | | | | | |
| | | Ter239 | | PSYPNYDA | | | | | | | | |
| 769 | NM_003119.2: | NP_003110.1: | 29159 | RFLQLGAKVPKGALLL | 1146 | HLA- | 65.83 | 0 | 2 | No | No | NA |
| | c.1053del | p.Gly352Alafs | | GPPGCGKTLLAKAVAT | | DRB1*01:02 | | | | | | |
| | | Ter87 | | EAQVPFLA | | | | | | | | |
| 770 | NM_001031734.3: | NP_001026904.2: | 29317 | AARGTWWNRPGGTSGS | 1242 | HLA- | 141.89 | 0 | 2 | No | No | NA |
| | c.118del | p.Val40Trpfs | | GEGVALGTTRKFQATG | | DRB1*08:04 | | | | | | |
| | | Ter32 | | SRPAGEED | | | | | | | | |
| 771 | NM_001098537.1: | NP_001092007.1: | 29431 | VAAGKAKKQVFYGEEE | 1243 | HLA- | 32.48 | 0 | 2 | No | No | NA |
| | c.1052del | p.Lys351Serfs | | RLKKPPRLQESCDSDH | | DRB1*01:02 | | | | | | |
| | | Ter25 | | GGGRPAAA | | | | | | | | |
| 772 | NM_015908.5: | NP_056992.4: | 29544 | DEHSSDPYHSGYEMPY | 1244 | HLA- | 219.11 | 0 | 2 | No | No | NA |
| | c.310del | p.Gly104Valfs | | AGGGGGPTYGPPQPWG | | DQA1*01:02/ | | | | | | |
| | | Ter45 | | HPDVHIMQ | | DQB1*06:02 | | | | | | |
| 773 | NM_001324098.1: | NP_001311027.1: | 29614 | TVDADTVTELAQVIVS | 1235 | HLA- | 208.96 | 0 | 2 | No | No | NA |
| | c.1154_1155insGG | p.Arg386Glyfs | | RGGRFLEAPVSGNQQL | | DRB1*04:04 | | | | | | |
| | | Ter21 | | SNDGMLVI | | | | | | | | |
| 774 | NM_032836.2: | NP_116225.2: | 29701 | MDDVPAPTPAPAPPAA | 1186 | HLA- | 58.7 | 0 | 2 | No | No | NA |
| | c.15del | p.Ala6Profs | | AAPRVPFHCS | | DRB1*08:04 | | | | | | |
| | | Ter61 | | | | | | | | | | |
| 775 | NM_021954.3: | NP_068773.2: | 29936 | ERQPPALKAYPAASTP | 1169 | HLA- | 106.76 | 0 | 2 | No | No | NA |
| | c.1056del | p.Ser353Alafs | | AAPSPVGSSSPPLAHE | | DRB1*01:02 | | | | | | |
| | | Ter46 | | AEAGAAPL | | | | | | | | |
| 776 | NM_002581.3: | NP_002572.2: | 30038 | LLDTKDQSHDLGLHVL | 1245 | HLA- | 134.84 | 0 | 2 | No | No | NA |
| | c.3422_3433del | p.Asn1141_Leu | | SCRNNPLIIPVVHDLS | | DRB1*01:02 | | | | | | |
| | | 1144del | | QPFYHSQAVRV | | | | | | | | |

Supplementary Table 12. List of other MHC-I neoAgs, with low predicted immunogenicity and low or no recurrency, obtained from the
computational methods in the discovery set.

| Mutant | | | | | | Micro- | Reference | | Altered | | |
|---|---|---|---|---|---|---|---|---|---|---|---|
| Epitope | SEQ | Gene | Chromo- | | | satellite | MS lengths | Variant | MS Length | Number deleted | Peptide |
| Sequence | ID NO | Name | some | Start | Stop | motif | (repeats) | Type | (repeats) | nucleotides | Length |
| KLFEKKYSV | 6 | TAF1B | chr2 | 10045013 | 10045014 | NA | NA | FS | NA | −1 | 9 |
| FLAPCNFYL | 16 | RNF128 | chrX | 105937255 | 105937255 | T | 8 | FS | 9 | 1 | 9 |
| FLIDDNFKV | 10 | TTLL10 | chr1 | 1116222 | 1116223 | G | 8 | FS | 7 | −1 | 9 |
| FLMDGFDEL | 11 | NLRP3 | chr1 | 247582212 | 247582213 | C | 5 | FS | 4 | −1 | 9 |
| FLWNSLLAV | 12 | ABCA7 | chr19 | 1056082 | 1056083 | G | 6 | FS | 5 | −1 | 9 |
| LTFCTNATI | 200 | SCNN1D | chr1 | 1221421 | 1221424 | TCT | 2 | inframe_ | 1 | −2 | 9 |
| | | | | | | | | del | | | |
| SLVRLSSCVP | 4 | TGFBR2 | chr3 | 30691871 | 30691872 | A | 10 | FS | 9 | −1 | 11 |
| V | | | | | | | | | | | |
| YLFAKAYLV | 201 | LANCL3 | chrX | 37431308 | 37431309 | G | 7 | FS | 6 | −1 | 9 |
| VPACSHVPM | 202 | CRIPAK | chr4 | 1388622 | 1388622 | AC | 2 | FS | 1 | 1 | 9 |
| VPTWSARLL | 203 | CRIPAK | chr4 | 1388622 | 1388622 | AC | 2 | FS | 1 | 1 | 9 |
| LLNPVTMNK | 204 | BCOR | chrX | 39922887 | 39922891 | NA | NA | FS | NA | −3 | 9 |
| SELLNPVTMN | 205 | BCOR | chrX | 39922887 | 39922891 | NA | NA | FS | NA | −3 | 11 |
| K | | | | | | | | | | | |
| LLNPVTMNK | 206 | BCOR | chrX | 39922887 | 39922891 | NA | NA | FS | NA | −3 | 10 |
| A | | | | | | | | | | | |
| ELLNPVTMN | 207 | BCOR | chrX | 39922887 | 39922891 | NA | NA | FS | NA | −3 | 10 |
| K | | | | | | | | | | | |
| SPAIPPRPL | 7 | BCOR | chrX | 39933761 | 39933763 | NA | NA | FS | NA | −1 | 9 |
| HPARHLCRL | 208 | ADGRB2 | chr1 | 32203322 | 32203322 | C | 5 | FS | 6 | 1 | 9 |
| APSLTPMHSL | 209 | GJA1 | chr6 | 121768924 | 121768925 | NA | NA | FS | NA | −1 | 10 |
| SPMITRIL | 210 | GJA1 | chr6 | 121768924 | 121768925 | NA | NA | FS | NA | −1 | 8 |
| TPMHSLLISP | 211 | GJA1 | chr6 | 121768924 | 121768925 | NA | NA | FS | NA | −1 | 11 |
| M | | | | | | | | | | | |
| TPMHSLLIS | 212 | GJA1 | chr6 | 121768924 | 121768925 | NA | NA | FS | NA | −1 | 9 |
| RLYKYDHNF | 213 | CTSC | chr11 | 88068107 | 88068108 | T | 6 | FS | 5 | −1 | 10 |
| V | | | | | | | | | | | |
| RQMLPTLSTL | 214 | SPECC1 | chr17 | 20108262 | 20108263 | A | 8 | FS | 7 | −1 | 10 |
| RLDKGNFAG | 215 | CELSR2 | chr1 | 109803706 | 109803707 | G | 3 | FS | 2 | −1 | 10 |
| A | | | | | | | | | | | |
| MLLELSPAQL | 216 | UBR5 | chr8 | 103289348 | 103289349 | A | 8 | FS | 7 | −1 | 10 |
| MLLELSPAQL | 216 | UBR5 | chr8 | 103289348 | 103289349 | T | 8 | FS | 7 | −1 | 10 |
| RPRMRAASPL | 13 | CAMKK2 | chr12 | 121678626 | 121678626 | C | 6 | FS | 7 | 1 | 10 |
| RPLCSSLGPL | 15 | CHAT | chr10 | 50827791 | 50827792 | C | 5 | FS | 4 | −1 | 10 |
| KPIAGRHTL | 8 | ZNF684 | chr1 | 41012499 | 41012499 | A | 5 | FS | 7 | 1 | 9 |
| TAPVASASPK | 17 | RBM15 | chr1 | 110882872 | 110882873 | C | 6 | FS | 5 | −1 | 11 |
| L | | | | | | | | | | | |
| RPVRGRGSL | 14 | DIDO1 | chr20 | 61512626 | 61512627 | C | 5 | FS | 4 | −1 | 9 |
| SMMPPPPAL | 2 | TCF7L2 | chr10 | 114925316 | 114925317 | A | 9 | FS | 8 | −1 | 9 |
| HPPPPCLLLL | 217 | SCAF4 | chr21 | 33073335 | 33073336 | C | 6 | FS | 5 | −1 | 10 |

| SEQ | | | Immuno- | | SEQ | | Binding | Tumor | Sample | | Elispot |
|---|---|---|---|---|---|---|---|---|---|---|---|
| ID | | | genicity | Wildtype | ID | HLA | Affinity | Abundance | Re- | Elispot | re- |
| NO | HGVSc | HGVSp | Score | sequence | NO | Allele | (nM) | (TPM) | currence | tested | active |
| 6 | NM_005680.2: | NP_005671.2:p. | 16889 | YGRDRGIFGIESWPDYEDI | 962 | HLA- | 3.8 | 7.27 | 2 | Tested | No |
| | c.834del | Tyr278Ter | | YKKTIEVGTFLDLPRFPDIT | | A*02: | | | | | |
| | | | | E | | 01 | | | | | |
| 16 | NM_024539.3: | NP_078815.3:p. | 77072 | MNQENRSSFFWLLVIFTFL | 963 | HLA- | 1.9 | 11.79 | 1 | Tested | No |
| | c.31dup | Trp11LeufsTer11 | | LKITASFSMSAY | | A*02: | | | | | |
| | | | | | | 01 | | | | | |
| 10 | NM_001130045.1: | NP_001123517.1: | 55580 | STLRGRARAMSKASKVPG | 964 | HLA- | 1.9 | 0.93 | 2 | Tested | Yes |
| | c.745del | p.Val249SerfsTer31 | | GVQARLEKDAAAPALEDL | | C*02: | | | | | |
| | | | | PWTS | | 02 | | | | | |
| 11 | NM_001079821.2: | NP_001073289.1: | 62255 | LKKFKMHLEDYPPQKGCIP | 965 | HLA- | 1.9 | 1.32 | 1 | Tested | No |
| | c.121del | p.Leu41SerfsTer14 | | LPRGQTEKADHVDLATLM | | A*02: | | | | | |
| | | | | IDF | | 01 | | | | | |
| 12 | NM_019112.3: | NP_061985.2:p. | 64179 | GLKTKKWVNEVRYGGFSL | 966 | HLA- | 2.2 | 6.39 | 1 | Tested | No |
| | c.4262del | Gly1421AlafsTer23 | | GGRDPGLPSGQELGRSVEE | | A*02: | | | | | |
| | | | | LWA | | 01 | | | | | |
| 200 | NM_001130413.3: | NP_001123885.2: | 34240 | GHQEGLVELPASFRELLTF | 967 | HLA- | 14.8 | 1.00E−04 | 2 | Tested | No |
| | c.680_682del | p.Phe227del | | FCTNATIHGAIRLVCSRGN | | B*15: | | | | | |
| | | | | RL | | 17 | | | | | |
| 4 | NM_001024847.2: | NP_001020018.1: | 1961 | HDFILEDAASPKCIMKEKK | 884 | HLA- | 55.3 | 7.63 | 5 | Tested | No |
| | c.458del | p.Lys153SerfsTer35 | | KPGETFFMCSCSSDECNDN | | A*02: | | | | | |
| | | | | II | | 01 | | | | | |
| 201 | NM_001170331.1: | NP_001163802.1: | 77068 | GGGAEARGATAGASACQ | 968 | HLA- | 1.7 | 0.19 | 1 | Tested | No |
| | c.192del | p.Leu65PhefsTer129 | | GGLYGGVAGVAYMLYHV | | A*02: | | | | | |
| | | | | SQSPLF | | 01 | | | | | |
| 202 | NM_175918.3: | NP_787114.2:p. | 62476 | DVECHLLTHVPMWSARLL | 969 | HLA- | 10.2 | 121.38 | 1 | Tested | No |
| | c.325_326dup | Cys110ArgfsTer81 | | TCPCGVPACSHVPMRSAR | | B*35: | | | | | |
| | | | | LLTR | | 01 | | | | | |
| 203 | NM_175918.3: | NP_787114.2:p. | 132827 | DVECHLLTHVPMWSARLL | 969 | HLA- | 941.1 | 121.38 | 1 | Tested | No |
| | c.325_326dup | Cys110ArgfsTer81 | | TCPCGVPACSHVPMRSAR | | B*35: | | | | | |
| | | | | LLTR | | 01 | | | | | |
| 204 | NM_00112338 | NP_001116857.1: | 70858 | EEKPGRKRAEAKGNRSWS | 970 | HLA- | 10.2 | 7.89 | 1 | Tested | No |
| | 5.1:c.3817_ | p.Glu1273LeufsTer20 | | EESLKPSDNEQGLPVFSGS | | A*03: | | | | | |
| | 3820del | | | PPM | | 01 | | | | | |
| 205 | NM_00112338 | NP_001116857.1: | 81099 | EEKPGRKRAEAKGNRSWS | 970 | HLA- | 59 | 7.89 | 1 | Tested | No |
| | 5.1:c.3817_ | p.Glu1273LeufsTer20 | | EESLKPSDNEQGLPVFSGS | | A*03: | | | | | |
| | 3820del | | | PPM | | 01 | | | | | |
| 206 | NM_00112338 | NP_001116857.1: | 87472 | EEKPGRKRAEAKGNRSWS | 970 | HLA- | 98.1 | 7.89 | 1 | Tested | No |
| | 5.1:c.3817_ | p.Glu1273LeufsTer20 | | EESLKPSDNEQGLPVFSGS | | A*03: | | | | | |
| | 3820del | | | PPM | | 01 | | | | | |
| 207 | NM_00112338 | NP_001116857.1: | 76661 | EEKPGRKRAEAKGNRSWS | 970 | HLA- | 36.7 | 7.89 | 1 | Tested | No |
| | 5.1:c.3817_ | p.Glu1273LeufsTer20 | | EESLKPSDNEQGLPVFSGS | | A*03: | | | | | |
| | 3820del | | | PPM | | 01 | | | | | |
| 7 | NM_00112338 | NP_001116857.1: | 53128 | SLASPMRLSTPSASPAIPPL | 971 | HLA- | 5.9 | 3.7 | 1 | Tested | No |
| | 5.1:c.836_ | p.Leu279ArgfsTer21 | | VHCADKSLPWKMGVSPG | | B*07: | | | | | |
| | 837del | | | NPV | | 02 | | | | | |
| 208 | NM_001294335.1: | NP_001281264.1: | 54855 | LAQPPKDLTLELAGSPSVP | 972 | HLA- | 26.8 | 1.46 | 1 | Tested | No |
| | c.2806dup | p.Leu936ProfsTer29 | | LVIGCAVSCMALLTLLAIY | | B*07: | | | | | |
| | | | | AA | | 02 | | | | Tested | |
| 209 | NM_000165.3: | NP_000156.1:p. | 77153 | DRNNSSCRNYNKQASEQN | 973 | HLA- | 27.6 | 43.46 | 1 | | No |
| | c.932del | Ala311ValfsTer37 | | WANYSAEQNRMGQAGSTI | | B*07: | | | | Tested | |
| | | | | SNSH | | 02 | | | | | |
| 210 | NM_000165.3: | NP_000156.1:p. | 74261 | DRNNSSCRNYNKQASEQN | 973 | HLA- | 15. | 43.46 | 1 | Tested | No |
| | c.932del | Ala311ValfsTer37 | | WANYSAEQNRMGQAGSTI | | B*07: | | | | | |
| | | | | SNSH | | 02 | | | | | |
| 211 | NM_000165.3: | NP_000156.1:p. | 77007 | DRNNSSCRNYNKQASEQN | 973 | HLA- | 26.9 | 43.46 | 1 | Tested | No |
| | c.932del | Ala311ValfsTer37 | | WANYSAEQNRMGQAGSTI | | B*07: | | | | | |
| | | | | SNSH | | 02 | | | | | |
| 212 | NM_000165.3: | NP_000156.1:p. | 126154 | DRNNSSCRNYNKQASEQN | 973 | HLA- | 530.9 | 43.46 | 1 | Tested | No |
| | c.932del | Ala311ValfsTer37 | | WANYSAEQNRMGQAGSTI | | B*07: | | | | | |
| | | | | SNSH | | 02 | | | | | |
| 213 | NM_001814.4: | NP_001805.3:p.P | 497 | IIYNQGFEIVLNDYKWFAF | 799 | HLA- | 33. | 77.33 | 1 | Tested | No |
| | c.315del | he105LeufsTer10 | | FKYKEEGSKVTTYCNETM | | A*02: | | | | | |
| | | | | TGW | | 01 | | | | | |
| 214 | NM_00124343 | NP_001230368.1: | 769 | SFGSPTGNQMSSDIDEYKK | 817 | HLA- | 7.3 | 5.55 | 3 | Tested | No |
| | 9.1:c.908del | p.Asn303ThrfsTer | | NIHGNALRTSGSSSSDVTK | | B*15: | | | | | |
| | | 63 | | AS | | 03 | | | | | |
| 215 | NM_001408.2: | NP_001399.1:p. | 140624 | DGYTGEHCEVSARSGRCT | 974 | HLA- | 983.9 | 1.67 | 1 | Tested | No |
| | c.4004del | Gly1335ValfsTer33 | | PGVCKNGGTCVNLLVGGF | | A*02: | | | | | |
| | | | | KCDC | | 01 | | | | | |
| 216 | NM_015902.5: | NP_056986.2:p. | 150 | MSYAANLKNVMNMQNRQ | 797 | HLA- | 12 | 61.73 | 4 | Tested | No |
| | c.6360del | Glu2121LysfsTer28 | | KKEGEEQPVLPEETESSKP | | A*02: | | | | | |
| | | | | GPSA | | 01 | | | | | |
| 216 | NM_015902.5: | NP_056986.2:p. | 150 | MSYAANLKNVMNMQNRQ | 797 | HLA- | 12 | 61.73 | 4 | Tested | No |
| | c.6360del | Glu2121LysfsTer28 | | KKEGEEQPVLPEETESSKP | | A*02: | | | | | |
| | | | | GPSA | | 01 | | | | | |
| 13 | NM_006549.3: | NP_006540.3:p. | 69164 | SELKEARQRRQPPGHRPAP | 975 | HLA- | 2 | 23.89 | 2 | Tested | No |
| | c.1642dup | Arg548ProfsTer66 | | RGGGGSALVRGSPCVESC | | B*07: | | | | | |
| | | | | WAP | | 02 | | | | | |
| 15 | NM_020549.4 | NP_065574.3:p. | 76087 | MAAKTPSSEESGLPKLPVP | 976 | HLA- | 6.4 | 0.54 | 1 | Tested | No |
| | c.413del | Pro138ArgfsTer62 | | PLQQTLATYLQCMRHLVS | | B*07: | | | | | |
| | | | | EEQ | | 02 | | | | | |
| 8 | NM_152373.3: | NP_689586.3:p. | 53278 | SYTVENAYECSECGKAFK | 977 | HLA- | 4.1 | 1.08 | 1 | Tested | No |
| | c.508_509dup | Lys171ArgfsTer93 | | KKFHFIRHEKNHTRKKPFE | | B*07: | | | | | |
| | | | | CND | | 02 | | | | | |
| 17 | NM_022768.4: | NP_073605.4:p. | 140585 | TYPPSASVVGASVGGHRH | 978 | HLA- | 978.6 | 8.51 | 1 | Tested | No |
| | c.851del | Pro284LeufsTer98 | | PPGGGGGQRSLSPGGAAL | | B*07: | | | | | |
| | | | | GYRD | | 02 | | | | | |
| 14 | NM_00119336 | NP_001180298.1: | 74888 | ADKPASLPPASQASNHRDP | 979 | HLA- | 2.4 | 46.37 | 1 | Tested | No |
| | 9.1:c.4681del | p.Arg1561GlyfsTer303 | | RQARRLATETGEGEGEPLS | | B*07: | | | | | |
| | | | | RL | | 02 | | | | | |
| 2 | NM_00114627 | NP_001139746.1: | 65 | ALFGLDRQTLWCKPCRRK | 788 | HLA- | 8.9 | 44.54 | 5 | Tested | No |
| | 4.1:c.1403del | p.Lys468SerfsTer23 | | KKCVRYIQGEGSCLSPPSS | | C*03: | | | | | |
| | | | | DGS | | 03 | | | | | |
| 217 | NM_020706.2: | NP_065757.1:p. | 140625 | TAQLKTTPTQPSEQKAAFP | 980 | HLA- | 995.6 | 41.16 | 2 | Tested | No |
| | c.749del | Pro250HisfsTer96 | | PPEQKTAFDKKLLDRFDY | | B*07: | | | | | |
| | | | | DDE | | 02 | | | | | |

Supplementary Table 13. Pooling of selected neoAgs for in vitro validation of immunogenicity using ELISpot assay..

| | | | | SEQ | | | | | | | | | |
|---|---|---|---|---|---|---|---|---|---|---|---|---|---|
| | | | | ID | | Re- | Molecular | | | Top | Top | | ELISpot |
| Pool | HLA Type | Gene | Description | NO | Rank | current | Weight | mg | MHC | Immunogenic | Recurrent | Others | reactive |
| 1 | HLA- | TAF1B | KLFEKKYSV | 6 | 58541 | 2 | 1141.37 | 4.9 | I | | | X | |
| | A*02:01 | | | | | | | | | | | | |
| | HLA- | RNF128 | FLAPCNFYL | 16 | 79323 | 1 | 1087.3 | 4.4 | I | | | X | |
| | A*02:01 | | | | | | | | | | | | |
| | HLA- | TTLL10 | FLIDDNFKV | 10 | 77276 | 2 | 1110.27 | 4.8 | I | | | X | yes |
| | A*02:01 | | | | | | | | | | | | |
| | HLA- | NLRP3 | FLMDGFDEL | 11 | 63244 | 1 | 1086.22 | 4.2 | I | | | X | |
| | A*02:01 | | | | | | | | | | | | |
| | HLA- | WDR6 | FMNSTVFHV | 3 | 904 | 2 | 1081.25 | 4.8 | I | X | | | |
| | A*02:01 | | | | | | | | | | | | |
| | HLA- | ABCA7 | FLWNSLLAV | 12 | 65469 | 1 | 1062.27 | 4.8 | I | | | X | |
| | A*02:01 | | | | | | | | | | | | |
| | HLA- | SCNN1D | FLGHHSFSV | | 128784 | 2 | 1030.14 | 4.9 | I | | | X | |
| | A*02:01 | | | | | | | | | | | | |
| | HLA- | MARCKS | FLQEVFQA | 5 | 74290 | 4 | 981.11 | 4.7 | I | | X | | |
| | A*02:01 | | | | | | | | | | | | |
| | HLA- | TGFBR2 | SLVRLSSCVPV | 4 | 1927 | 5 | 1159.41 | 4.1 | I | | | X | |
| | A*02:01 | | | | | | | | | | | | |
| | HLA- | LANCL3 | YLFAKAYLV | 201 | 77068 | 1 | 1087.32 | 4.7 | I | | | X | |
| | A*02:01 | | | | | | | | | | | | |
| 2 | HLA- | CRIPAK-2 | VPACSHVPM | 202 | 63413 | 1 | 940.15 | 4.1 | I | | | X | |
| | B*35:01 | | | | | | | | | | | | |
| | HLA- | CRIPAK-1 | VPTWSARLL | 203 | 138880 | 1 | 1042.24 | 4.6 | I | | | X | |
| | B*35:01 | | | | | | | | | | | | |
| | HLA- | BCOR-4 | LLNPVTMNK | 204 | 72894 | 1 | 1029.26 | 4.9 | I | | | | |
| | A*03:01 | | | | | | | | | | | | |
| | HLA- | BCOR-2 | SELLNPVTMNK | 205 | 83980 | 1 | 1245.45 | 4.9 | I | | | X | |
| | A*03:01 | | | | | | | | | | | | |
| | HLA- | BCOR-1 | LLNPVTMNKA | 206 | 90887 | 1 | 1100.34 | 4 | I | | | X | yes |
| | A*03:01 | | | | | | | | | | | | |
| | HLA- | BCOR-3 | ELLNPVTMNK | 207 | 79229 | 1 | 1158.37 | 4.1 | I | | | X | |
| | A*03:01 | | | | | | | | | | | | |
| | HLA- | ADGRB2 | HPARHLCRL | 208 | 55619 | 1 | 1102.32 | 4.8 | I | | | X | |
| | B*07:02 | | | | | | | | | | | | |
| | HLA- | GJA1-2 | APSLTPMHSL | 209 | 79637 | 1 | 1053.24 | 4.1 | I | | | X | |
| | B*07:02 | | | | | | | | | | | | |
| | HLA- | GJA1-4 | SPMITRIL | 210 | 76488 | 1 | 930.17 | 4 | I | | | X | |
| | B*07:02 | | | | | | | | | | | | |
| | HLA- | GJA1-3 | TPMHSLLISPM | 211 | 79465 | 1 | 1226.52 | 4 | I | | | X | |
| | B*07:02 | | | | | | | | | | | | |
| | HLA- | CTSC | IQKSWTATTY | 39 | 139 | 1 | 1198.33 | 4.7 | I | X | | | yes |
| | B*15:03 | | | | | | | | | | | | |
| 3 | HLA- | CLCA1 | SLINLTWTA | 66 | 413 | 1 | 1018.17 | 4 | I | X | | | yes |
| | A*02:01 | | | | | | | | | | | | |
| | HLA- | SMARCAD1 | NMIQVLMSV | 49 | 238 | 1 | 1034.3 | 4.7 | I | X | | | |
| | A*02:01 | | | | | | | | | | | | |
| | HLA- | CTSC | RLYKYDHNFV | 213 | 502 | 1 | 1354.52 | 4.6 | I | | | X | |
| | A*02:01 | | | | | | | | | | | | |
| | HLA- | ALG8 | FLALNQLPQV | 112 | 1139 | 1 | 1142.35 | 4 | I | X | | | |
| | A*02:01 | | | | | | | | | | | | |
| | HLA- | SPECC1 | RQMLPTLSTL | 214 | 706 | 4 | 1159.41 | 4.8 | I | | | X | |
| | B*15:03 | | | | | | | | | | | | |
| | HLA- | TGFBR2 | SQKNITPAI | 122 | 1605 | 5 | 971.11 | 4.9 | I | | X | | |
| | B*15:03 | | | | | | | | | | | | |
| | HLA- | CELSR2 | RLDKGNFAGA | 215 | 147448 | 1 | 1048.16 | 4.8 | I | | | X | yes |
| | A*02:01 | | | | | | | | | | | | |
| | HLA- | UBR5 | MLLELSPAQL | 216 | 147 | 5 | 1114.36 | 4.7 | I | | | X | |
| | A*02:01 | | | | | | | | | | | | |
| | HLA- | PAWR | APREGAAATPL | 18 | 1 | 1 | 1053.18 | 4.9 | I | X | | | |
| | B*07:02 | | | | | | | | | | | | |
| | HLA- | GJA1-1 | TPMHSLLIS | 212 | 132175 | 1 | 998.2 | 4.3 | I | | | X | |
| | B*07:02 | | | | | | | | | | | | |
| 4 | HLA- | GOLIM4 | KMMKILMIK | 19 | 2 | 2 | 1135.6 | 4.8 | I | X | | | |
| | A*03:01 | | | | | | | | | | | | |
| | HLA- | SPINK5 | LSAPEKITLF | 20 | 8 | 3 | 1118.33 | 4.6 | I | X | X | | |
| | B*15:17 | | | | | | | | | | | | |
| | HLA- | P4HB | SLWSSMPHGV | 23 | 19 | 2 | 1100.25 | 4.5 | I | X | | | |
| | A*02:01 | | | | | | | | | | | | |
| | HLA- | SEC31A | MLLRLNLRK | 29 | 62 | 3 | 1156.49 | 4.6 | I | X | X | | |
| | A*03:01 | | | | | | | | | | | | |
| | HLA- | UBR5 | RLYVPLYSSK | 37 | 119 | 4 | 1225.44 | 4.2 | I | X | X | | yes |
| | A*03:01 | | | | | | | | | | | | |
| | HLA- | SPECC1 | KTYMEMHY | 57 | 345 | 3 | 1102.29 | 4.6 | I | X | X | | yes |
| | B*15:17 | | | | | | | | | | | | |
| | HLA- | PTEN | KANRYFSPNF | 62 | 391 | 3 | 1243.38 | 4.8 | I | X | X | | |
| | B*15:17 | | | | | | | | | | | | |
| | HLA- | TMEM94 | KPLWRKSPL | 137 | 10241 | 4 | 1124.38 | 4.4 | I | | X | | |
| | B*07:02 | | | | | | | | | | | | |
| | HLA- | CELSR1 | SPSRSTTAPV | 134 | 3456 | 4 | 1002.09 | 4 | I | | X | | |
| | B*07:02 | | | | | | | | | | | | |
| | HLA- | NOL4L | AMAENILAA | 150 | 4368 | 3 | 903.06 | 4.7 | I | | X | | |
| | A*02:01 | | | | | | | | | | | | |
| | HLA- | GPBP1L1 | KLSSVVPSV | 22 | 13 | 1 | 915.09 | 4.6 | I | X | | | |
| | A*02:01 | | | | | | | | | | | | |
| 5 | HLA- | TCF7L2 | IAQPSTSSL | 28 | 53 | 5 | 902.99 | 4.9 | I | X | X | | |
| | C*03:03 | | | | | | | | | | | | |
| | HLA- | WDTC1 | FLATSGIDPV | 44 | 178 | 6 | 1019.15 | 4.9 | I | X | X | | |
| | A*02:01 | | | | | | | | | | | | |
| | HLA- | AP1S1 | RSVLEEMGL | 58 | 360 | 4 | 1033.21 | 4.6 | I | X | X | | |
| | B*15:17 | | | | | | | | | | | | |
| | HLA- | ELMSA | GMVPLIIPV | 116 | 14559 | 7 | 938.23 | 4.2 | I | | X | | |
| | A*02:01 | N1 | | | | | | | | | | | |
| | HLA- | BMPR2 | TPQDSRQVL | 117 | 3845 | 6 | 1043.14 | 4.9 | I | | X | | |
| | B*07:02 | | | | | | | | | | | | |
| | HLA- | ARID1A | LTHPAHQPL | 124 | 5556 | 5 | 1013.16 | 4.2 | I | | X | | |
| | B*15:17 | | | | | | | | | | | | |
| | HLA- | CASP5 | LKLCSKVSF | 132 | 1697 | 5 | 1024.28 | 4.3 | I | | X | | |
| | B*15:03 | | | | | | | | | | | | |
| | HLA- | RNF43 | TQLARFFPI | 24 | 26 | 8 | 1092.3 | 4.1 | I | X | X | | yes |
| | A*02:01 | | | | | | | | | | | | |
| | HLA- | TCF20 | RVPAHASTSI | 27 | 42 | 5 | 1038.16 | 4.9 | I | X | X | | yes |
| | B*07:02 | | | | | | | | | | | | |
| | HLA- | SCAF4 | HPPPPCLLLL | 217 | 147447 | 2 | 1099.4 | 4.1 | I | | | X | |
| | B*07:02 | | | | | | | | | | | | |
| 6 | HLA- | BCOR | SPAIPPRPL | 7 | 53801 | 1 | 947.14 | 4.4 | I | | | X | yes |
| | B*07:02 | | | | | | | | | | | | |
| | HLA- | CAMKK2 | RPRMRAASPL | 13 | 77293 | 2 | 1154.4 | 4.9 | I | | | | |
| | B*07:02 | | | | | | | | | | | | |
| | HLA- | CHAT | RPLCSSLGPL | 15 | 78296 | 1 | 1042.26 | 4.1 | I | | | X | |
| | B*07:02 | | | | | | | | | | | | |
| | HLA- | ZNF684 | KPIAGRHTL | 8 | 53955 | 1 | 992.18 | 4.4 | I | | | X | |
| | B*07:02 | | | | | | | | | | | | |
| | HLA- | RBM15 | TAPVASASPKL | 17 | 147404 | 1 | 1041.2 | 4.6 | I | | | X | |
| | B*07:02 | | | | | | | | | | | | |
| | HLA- | DIDO1 | RPVRGRGSL | 14 | 77059 | 1 | 997.16 | 4.7 | I | | | X | |
| | B*07:02 | | | | | | | | | | | | |
| | HLA- | USP9Y | YMMDDLELI | 1 | 17 | 1 | 1142 | 4 | I | X | | | |
| | A*02:01 | | | | | | | | | | | | |
| | HLA- | TCF7L2 | SMMPPPPAL | 2 | 65 | 5 | 940 | 4.4 | I | | | X | |
| | C*03:03 | | | | | | | | | | | | |
| 7 | HLA- | TET2 | YLRFIKSLAERT | 231 | 301 | 2 | 1814.17 | 4.2 | II | X | | | |
| | DRB1*07:01 | | MSV | | | | | | | | | | |
| | HLA- | CAMTA2 | SPPLHLCQPL | 95 | 935 | 3 | 1104.33 | 4 | I | X | X | | |
| | B*07:02 | | | | | | | | | | | | |
| | HLA- | CLDN4 | ASLAHSDNF | 106 | 1072 | 3 | 960.99 | 4.3 | I | X | X | | |
| | B*15:17 | | | | | | | | | | | | |
| | HLA- | STAMBPL1 | KRAFIHTPR | 131 | 1679 | 4 | 1125.33 | 4.4 | I | | X | | |
| | B*27:05 | | | | | | | | | | | | |
| | HLA- | PRDM2 | KVDTHHLQV | 133 | 2434 | 4 | 1076.21 | 4.7 | I | | X | | |
| | C*05:01 | | | | | | | | | | | | |
| | HLA- | CHD3 | KADQSESSL | 123 | 2604 | 5 | 963.99 | 4.2 | I | | X | | |
| | C*05:01 | | | | | | | | | | | | |
| | HLA- | CNKSR1 | RRPLRSWTPR | 152 | 4500 | 3 | 1324.54 | 4.6 | I | | X | | |
| | B*27:05 | | | | | | | | | | | | |
| | HLA- | COL9A2 | RAWRAGMPL | 153 | 4763 | 3 | 1057.28 | 4.6 | I | | X | | |
| | B*15:17 | | | | | | | | | | | | |
| | HLA- | PGD | MPCFTTALLL | 155 | 5554 | 3 | 1109.41 | 4.2 | I | | X | | |
| | B*51:01 | | | | | | | | | | | | |
| | HLA- | LARP4B | RTLLVTCILY | 125 | 5649 | 5 | 1194.49 | 3.6 | I | | X | | |
| | A*30:02 | | | | | | | | | | | | |
| 8 | HLA- | CRIM1 | QTIEERLTW | 156 | 5940 | 3 | 1175.3 | 4.9 | I | | X | | yes |
| | B*15:17 | | | | | | | | | | | | |
| | HLA- | AASDH | VMANVLTLNL | 157 | 6022 | 3 | 1087.34 | 4 | I | | X | | yes |
| | A*02:01 | | | | | | | | | | | | |
| | HLA- | CCDC186 | VLEDTLLKI | 158 | 6671 | 3 | 1043.26 | 4 | I | | X | | yes |
| | A*02:01 | | | | | | | | | | | | |
| | HLA- | HOXA11 | AGIGWGASY | 160 | 7459 | 3 | 880.95 | 4.3 | I | | X | | yes |
| | A*30:02 | | | | | | | | | | | | |
| | HLA- | ZFP36L2 | RMASTSCAA | 161 | 7672 | 3 | 897.04 | 4.1 | I | | X | | |
| | A*02:01 | | | | | | | | | | | | |
| | HLA- | BCORL1 | WSWCGTSQTY | 136 | 8277 | 4 | 1218.3 | 4.7 | I | | X | | |
| | B*15:17 | | | | | | | | | | | | |
| | HLA- | R3HDM2 | WLPKMPPFV | 163 | 10387 | 3 | 1114.41 | 4.9 | I | | X | | yes |
| | A*02:01 | | | | | | | | | | | | |
| | HLA- | MRI1 | SQNWGSLPL | 164 | 13130 | 3 | 1001.1 | 4.8 | I | | X | | |
| | B*15:01 | | | | | | | | | | | | |
| | HLA- | SLC23A2 | RLSCAPPPI | 138 | 15302 | 4 | 953.17 | 4.3 | I | | X | | yes |
| | A*02:01 | | | | | | | | | | | | |
| | HLA- | PTEN | ANRYFSPNFKV | 290 | 2546 | 3 | 1894.19 | 4 | II | X | X | | |
| | DRB1*13:02 | | KLYF | | | | | | | | | | |
| 9 | HLA- | MYO10 | GLLHAVQEKL | 165 | 18568 | 3 | 1107.31 | 4.9 | I | | X | | |
| | A*02:01 | | | | | | | | | | | | |
| | HLA- | MAPRE3 | LSNVAPPAF | 168 | 23926 | 3 | 915.05 | 4.2 | I | | X | | yes |
| | B*15:17 | | | | | | | | | | | | |
| | HLA- | C22orf24 | KVPFFSALK | 169 | 26241 | 3 | 1036.27 | 4.1 | I | | X | | |
| | A*03:26 | | | | | | | | | | | | |
| | HLA- | PLOD3 | RFCPASCSGCY | 170 | 27472 | 3 | 1193.38 | 4.6 | I | | X | | yes |
| | A*30:02 | | | | | | | | | | | | |
| | HLA- | USF2 | RTHPYSPKK | 171 | 28137 | 3 | 1113.28 | 4.3 | I | | X | | |
| | A*03:26 | | | | | | | | | | | | |
| | HLA- | CLCA2 | VSNIAQAPLY | 172 | 29458 | 3 | 1075.22 | 4.4 | I | | X | | |
| | B*15:17 | | | | | | | | | | | | |
| | HLA- | TMEM132D | LSSWFSPTV | 139 | 29938 | 4 | 1023.14 | 4.7 | I | | X | | |
| | B*15:17 | | | | | | | | | | | | |
| | HLA- | OR1K1 | YVAIRPLPY | 173 | 30248 | 3 | 1091.31 | 4.8 | I | | X | | |
| | B*15:17 | | | | | | | | | | | | |
| | HLA- | AP1S1 | KKSVLKAIEQAD | 335 | 11891 | 4 | 1684 | 4.1 | II | | X | | |
| | DRB1*10:01 | | LLQ | | | | | | | | | | |
| | HLA- | MARCKS | KEALFLQEVFQA | 336 | 16707 | 4 | 1821.09 | 4.5 | II | | X | | |
| | DRB1*04:05 | | ERL | | | | | | | | | | |
| 10 | HLA- | OR7E24 | ILFFFSSK | 175 | 32261 | 3 | 988.18 | 4 | I | | X | | |
| | A*03:26 | | | | | | | | | | | | |
| | HLA- | DAZAP1 | HNVQGFHPY | 178 | 34622 | 3 | 1098.18 | 4 | I | | X | | |
| | A*30:02 | | | | | | | | | | | | |
| | HLA- | CACNA1G | SMAASPSPK | 179 | 34903 | 3 | 875.01 | 4.5 | I | | X | | |
| | A*03:26 | | | | | | | | | | | | |
| | HLA- | OR4M1 | SWMGGLHSFY | 121 | 48030 | 6 | 1184.33 | 4.2 | I | | X | | |
| | A*30:02 | | | | | | | | | | | | |
| | HLA- | RNF43 | QLARFFPITPPV | 234 | 381 | 8 | 1822.17 | 4.4 | II | X | X | | |
| | DRB1*04:05 | | WHI | | | | | | | | | | |
| | HLA- | MARCKS | RSAFPSRSL | 126 | 6932 | 5 | 1020.15 | 4.5 | I | | X | | |
| | B*15:17 | | | | | | | | | | | | |
| | HLA- | USP35 | TPRKLVGRAV | 162 | 9425 | 3 | 1096.33 | 4.2 | I | | X | | |
| | B*07:02 | | | | | | | | | | | | |
| | HLA- | ZBED6CL | LAYWEKREAW | 177 | 34279 | 3 | 1351.51 | 4 | I | | X | | |
| | B*15:17 | | | | | | | | | | | | |
| | HLA- | CAD | ATLVTPPTRY | 166 | 21546 | 3 | 1118.29 | 4.3 | I | | X | | |
| | A*30:02 | | | | | | | | | | | | |
| | HLA- | MICAL3 | RFPLLMMWRTP | 321 | 10447 | 6 | 1937.41 | 1 | II | | X | | |
| | DRB1*13:01 | | MTTR | | | | | | | | | | |
| | HLA- | OR4M1 | ILVALSWMGGL | 325 | 143257 | 6 | 1694.01 | 3 | II | | X | | |
| | DRB1*01:01 | | HSFY | | | | | | | | | | |
| 11 | HLA- | ELMSAN1 | LRGGVIQSTRRR | 319 | 36602 | 7 | 1782.08 | 4 | II | | X | | |
| | DRB1*13:01 | | RRA | | | | | | | | | | |
| | HLA- | ACVR2A | EDMQEVVVHKK | 320 | 4522 | 6 | 1815.11 | 4.2 | II | | X | | |
| | DRB1*13:01 | | RGLF | | | | | | | | | | |
| | HLA- | TCF7L2 | PALLLAEATHK | 229 | 263 | 5 | 1505.76 | 4.7 | II | X | X | | |
| | DRB1*13:01 | | ASAL | | | | | | | | | | |
| | HLA- | P4HB | RWMVLRNSWR | 218 | 1 | 2 | 1932.34 | 4.9 | II | X | | | yes |
| | DRB1*13:01 | | AVARM | | | | | | | | | | |
| | HLA- | SLC4A11 | SLDNVLRTMLR | 230 | 288 | 2 | 1848.19 | 4.5 | II | X | | | |
| | DRB1*13:01 | | RFAR | | | | | | | | | | |
| | HLA- | RNF213 | LSSPFREQM | 38 | 143 | 3 | 1094.25 | 4.1 | I | X | X | | |
| | B*15:17 | | | | | | | | | | | | |
| | HLA- | ACVR2A | VVHKKRGLF | 127 | 10038 | 5 | 1083.34 | 4.2 | I | | X | | |
| | B*15:17 | | | | | | | | | | | | |
| | HLA- | MFRP | TSSPRTMSW | 120 | 37818 | 6 | 1052.17 | 4.8 | I | | X | | |
| | B*15:17 | | | | | | | | | | | | |
| | HLA- | MYCN | RTRSAWGDW | 141 | 36344 | 4 | 1134.21 | 4.2 | I | | X | | |
| | B*15:17 | | | | | | | | | | | | |
| | HLA- | KLHL29 | HTCKVCVSF | 176 | 34025 | 3 | 1023.24 | 4.1 | I | | X | | |
| | B*15:17 | | | | | | | | | | | | |
| 12 | HLA- | ASTE1 | RIPAVLRTEGEP | 323 | 51952 | 6 | 1688.93 | 4.1 | II | | X | | yes |
| | DRB1*07:01 | | LHT | | | | | | | | | | |
| | HLA- | USP9Y | FQVHFLKSGGLP | 221 | 15 | 1 | 1655 | 4 | II | X | | | yes |
| | DRB1*07:01 | | LVL | | | | | | | | | | |
| | HLA- | BTBD7 | PSKRSLLSVGNL | 248 | 734 | 2 | 1553.85 | 4.9 | II | X | | | |
| | DRB1*07:01 | | IGL | | | | | | | | | | |
| | HLA- | RABGAP1 | KKTVLSLVTISR | 260 | 1123 | 2 | 1704.11 | 4.1 | II | X | | | |
| | DRB1*07:01 | | FVL | | | | | | | | | | |
| | HLA- | TGFBR2 | KSLVRLSSCVPV | 311 | 3869 | 5 | 1603.01 | 4.9 | II | X | X | | |
| | DRB1*07:01 | | ALM | | | | | | | | | | |
| | HLA- | XYLT2 | RPACTCISM | 97 | 969 | 3 | 981.22 | 4.4 | I | X | X | | |
| | B*07:02 | | | | | | | | | | | | |
| | HLA- | OR52N5 | MYFFWPCSL | 174 | 32050 | 3 | 1193.44 | 4.5 | I | | X | | |
| | C*07:01 | | | | | | | | | | | | |
| | HLA- | FAM83D | YLGTPTWNC | 151 | 4370 | 3 | 1054.18 | 4.5 | I | | X | | yes |
| | A*02:01 | | | | | | | | | | | | |
| | HLA- | CDC7 | KLYEAVPQL | 159 | 7375 | 3 | 1060.25 | 4.5 | I | | X | | |
| | A*02:01 | | | | | | | | | | | | |
| | HLA- | SIN3A | HSWRFCTHIR | 154 | 5021 | 3 | 1342.54 | 4.4 | I | | X | | |
| | A*31:01 | | | | | | | | | | | | |
| 13 | HLA- | ATP8A2 | FGILNVLEFSSD | 324 | 133802 | 6 | 1753.02 | 4.3 | II | | X | | |
| | DRB1*01:01 | | RKK | | | | | | | | | | |
| | HLA- | ARID1A | LLHWRIGGGTPL | 328 | 19520 | 5 | 1606.87 | 4.7 | II | | X | | |
| | DRB1*01:01 | | SIS | | | | | | | | | | |
| | HLA- | BMPR2 | EIQLTMNDSKH | 329 | 31532 | 5 | 1772.98 | 4.9 | II | | X | | |
| | DRB1*13:01 | | KLES | | | | | | | | | | |
| | HLA- | SEC31A | QKKLMLLRLNL | 223 | 61 | 3 | 1888.47 | 4.1 | II | X | X | | yes |
| | DRB1*13:01 | | RKMC | | | | | | | | | | |
| | HLA- | TCERG1 | TKFITYRSKKLIQ | 238 | 461 | 2 | 1842.15 | 4.2 | II | X | | | |
| | DRB1*13:01 | | ES | | | | | | | | | | |
| | HLA- | TSPAN7 | IAFSQLIGM | 167 | 23216 | 3 | 979.2 | 4.5 | I | | X | | |
| | C*03:04 | | | | | | | | | | | | |
| | HLA- | DOCK3 | SQVWTAATLR | 115 | 34851 | 8 | 1132.28 | 4.5 | I | | X | | |
| | A*03:01 | | | | | | | | | | | | |
| | HLA- | COBLL1 | CRREYRVTM | 135 | 6489 | 4 | 1213.44 | 4.8 | I | | X | | |
| | C*06:02 | | | | | | | | | | | | |
| | HLA- | MICAL3 | RAWRRFPLL | 118 | 7352 | 6 | 1214.47 | 4.8 | I | | X | | |
| | C*16:01 | | | | | | | | | | | | |
| | HLA- | ASTE1 | VGMRETTGL | 119 | 24474 | 6 | 963.12 | 4 | I | | X | | |
| | C*16:01 | | | | | | | | | | | | |
| 14 | HLA- | TCF20 | TVEMRRWWTL | 250 | 785 | 5 | 2051.45 | 4.7 | II | X | X | | |
| | DRB1*15:01 | | VMEWK | | | | | | | | | | |
| | HLA- | UBR5 | RHVIKVLLGRK | 233 | 375 | 4 | 1855.25 | 4.7 | II | X | X | | |
| | DRB1*15:01 | | VNWH | | | | | | | | | | |
| | HLA- | CELSR1 | ALGLRILPPPLTS | 334 | 11687 | 4 | 1531.85 | 4.5 | II | | X | | |
| | DRB1*15:01 | | PS | | | | | | | | | | |
| | HLA- | WDTC1 | LEVMLLNMGYR | 239 | 476 | 6 | 1723.12 | 4.4 | II | X | X | | yes |
| | DRB1*15:01 | | ITGL | | | | | | | | | | |
| | HLA- | WDR74 | NSVIVGNTHGQL | 261 | 1133 | 2 | 1551.71 | 4.4 | II | X | | | |
| | DRB1*13:02 | | AEI | | | | | | | | | | |
| | HLA- | GOLIM4 | KTGLQLLRNHIE | 222 | 30 | 2 | 1792.1 | 4.1 | II | X | | | |
| | DRB1*13:02 | | ELK | | | | | | | | | | |
| | HLA- | SPECC1 | PVTPLRVQSVLL | 354 | 6292 | 3 | 1590.96 | 4 | II | | X | | |
| | DRB1*13:02 | | LGV | | | | | | | | | | |
| | HLA- | RAD50 | IDNIKRNHNLAL | 219 | 10 | 2 | 1761.99 | 4.6 | II | X | | | |
| | DRB1*13:02 | | GRQ | | | | | | | | | | |
| | HLA- | KMT2C | LINIHHRKNPLLP | 224 | 167 | 2 | 1852.27 | 4 | II | X | | | |
| | DRB1*13:02 | | MR | | | | | | | | | | |
| | HLA- | KLHL7 | TARISVNSNNVQ | 243 | 561 | 2 | 1615.79 | 4.1 | II | X | | | |
| | DRB1*13:02 | | SLL | | | | | | | | | | |
| | HLA- | SPINK5 | SINVLCVRASLIE | 240 | 499 | 3 | 1658.02 | 4.3 | II | X | X | | yes |
| | DRB1*13:02 | | KL | | | | | | | | | | |
| 15 | HLA- | CASP5 | GVRILKLCSKVS | 332 | 5659 | 4 | 1705.13 | 4.2 | II | | X | | |
| | DRB1*01:02 | | FRV | | | | | | | | | | |
| | HLA- | DOCK3 | PCASLLSTLSQPP | 319 | 78669 | 7 | 1538.77 | 2.3 | II | | X | | |
| | DRB1*04:03 | | PQ | | | | | | | | | | |
| | HLA- | DAZAP1 | FHPYRRYPPPAA | 256 | 936 | 2 | 1726.99 | 4.6 | II | X | | | |
| | DRB1*10:01 | | AAL | | | | | | | | | | |
| | HLA- | VPS13A | FEEIIKNDGALL | 236 | 442 | 2 | 1746.07 | 4.5 | II | X | | | |
| | DRB1*03:01 | | KKK | | | | | | | | | | |
| | HLA- | CHD3 | PVLFKADQSESS | 322 | 15344 | 6 | 1594.73 | 4.7 | II | | X | | |
| | DRB1*03:01 | | LSS | | | | | | | | | | |
| | HLA- | ANO10 | NONLYLVGASKI | 255 | 892 | 2 | 1720.05 | 2 | II | X | | | |
| | DRB1*07:01 | | RML | | | | | | | | | | |
| | HLA- | MFN2 | EDIEFHFSLGWT | 227 | 190 | 2 | 1824.07 | 4.3 | II | X | | | |
| | DRB1*07:01 | | MLV | | | | | | | | | | |
| | HLA- | WDR59 | NKKMLTALPPA | 251 | 792 | 2 | 1618.05 | 4.5 | II | X | | | |
| | DRB1*07:01 | | MTAM | | | | | | | | | | |
| | HLA- | ZFR | PKMQVTITLTSPI | 220 | 14 | 2 | 1698.09 | 4.5 | II | X | | | |
| | DRB1*07:01 | | IR | | | | | | | | | | |
| | HLA- | SLC22A9 | MSSIWGTMF | 140 | 32386 | 4 | 1059.27 | 4.7 | I | | X | | |
| | B*15:17 | | | | | | | | | | | | |

Supplementary Table 14. List of the validated ELISpot-reactive peptides

| Mutant | SEQ | | | | | Micro- | Reference | | Altered | Number | |
|---|---|---|---|---|---|---|---|---|---|---|---|
| Epitope | ID | Gene | Chromo- | | | satellite | MS lengths | Variant | MS Length | deleted | Peptide |
| Sequence | NO | Name | some | Start | Stop | motif | (repeats) | Type | (repeats) | nucleotides | Length |
| FLIDDNFKV | 10 | TTLL10 | chr1 | 1116222 | 1116223 | G | 8 | FS | 7 | -1 | 9 |
| RIPAVLRTEG | 323 | ASTE1 | chr3 | 130733046 | 130733047 | A | 11 | FS | 10 | -1 | 15 |
| EPLHT | | | | | | | | | | | |
| FQVHFLKSGG | 221 | USP9Y | chrY | 14847610 | 14847611 | T | 7 | FS | 6 | -1 | 15 |
| LPLVL | | | | | | | | | | | |
| FLATSGIDPV | 44 | WDTC1 | chr1 | 27621107 | 27621108 | G | 8 | FS | 7 | -1 | 10 |
| RVPAHASTSL | 27 | TCF20 | chr22 | 42564715 | 42564716 | C | 7 | FS | 6 | -1 | 10 |
| QTIEERLTW | 156 | CRIM1 | chr2 | 36764627 | 36764628 | C | 6 | FS | 5 | -1 | 9 |
| RLYVPLYSSK | 37 | UBR5 | chr8 | 103289348 | 103289349 | A | 8 | FS | 7 | -1 | 10 |
| LSNVAPPAF | 168 | MAPRE3 | chr2 | 27248516 | 27248517 | C | 8 | FS | 7 | -1 | 9 |
| LSAPEKITLF | 20 | SPINK5 | chr5 | 147499874 | 147499875 | A | 10 | FS | 9 | -1 | 10 |
| WLPKMPPFV | 163 | R3HDM2 | chr12 | 57648749 | 57648750 | G | 13 | FS | 12 | -1 | 9 |
| MLLRLNLRK | 29 | SEC31A | chr4 | 83785564 | 83785565 | A | 9 | FS | 8 | -1 | 9 |
| WSWCGTSQT | 136 | BCORL1 | chrX | 129190010 | 129190011 | C | 7 | FS | 6 | -1 | 10 |
| Y | | | | | | | | | | | |

| | | | | | | | | | | | |
|---|---|---|---|---|---|---|---|---|---|---|---|
| TQLARFFPI | 24 | RNF43 | chr17 | 56435160 | 56435161 | G | 7 | FS | 6 | -1 | 9 |
| KANRYFSPNF | 62 | PTEN | chr10 | 89720811 | 89720812 | A | 6 | FS | 5 | -1 | 10 |
| RLSCAPPPI | 138 | SLC23A2 | chr20 | 4850568 | 4850569 | C | 9 | FS | 8 | -1 | 9 |
| VMANVLTLN | 157 | AASDH | chr4 | 57220268 | 57220269 | T | 10 | FS | 9 | -1 | 10 |
| L | | | | | | | | | | | |
| AGIGWGASY | 160 | HOXA11 | chr7 | 27222461 | 27222462 | A | 9 | FS | 8 | -1 | 9 |
| YLGTPTWNC | 151 | FAM83D | chr20 | 37580942 | 37580943 | CA | 3 | FS | 2 | -1 | 9 |
| VLEDTLLKI | 158 | CCDC186 | chr10 | 115885657 | 115885658 | A | 6 | FS | 5 | -1 | 9 |
| SLWSSMPHG | 23 | P4HB | chr17 | 79803763 | 79803764 | A | 8 | FS | 7 | -1 | 10 |
| V | | | | | | | | | | | |
| IQKSWTATTY | 39 | CTSC | chr11 | 88068107 | 88068108 | T | 6 | FS | 5 | -1 | 10 |
| KTYMEMHY | 57 | SPECC1 | chr17 | 20108262 | 20108263 | A | 8 | FS | 7 | -1 | 8 |

| | | | | | | | | | | | | Present |
|---|---|---|---|---|---|---|---|---|---|---|---|---|
| SEQ | | | Immuno- | | SEQ | | Binding | Tumor | Sample | | | the Vali- |
| ID | | | genicity | Wildtype | ID | HLA | Affinity | Abundance | Recur- | Elispot | Elispot | dation |
| NO | HGVSc | HGVSp | Score | sequence | NO | Allele | (nM) | (TPM) | rence | tested | reactive | Set |
| 10 | NM_001130045.1: | NP_001123517.1: | 55580 | STLRGRARAMSKASKV | 964 | HLA- | 1.9 | 0.93 | 2 | Tested | Yes | No |
| | c.745del | p. | | PGGVQARLEKDAAAPA | | C*02: | | | | | | |
| | | Val249SerfsTer31 | | LEDLPWTS | | 02 | | | | | | |
| 323 | NM_001288950.1: | NP_001275879.1: | 51952 | SYAPAEIFLPKGRSNSK | 881 | HLA- | 359.76 | 4.09 | 6 | Tested | Yes | No |
| | c.1969del | p. | | KKRQKKQNTSCSKNRG | | DRB1* | | | | | | |
| | | Arg657GlyfsTer33 | | RTTAHTK | | 07:01 | | | | | | |
| 221 | NM_004654.3: | NP_004645.2: | 15 | DLINKFGTLNGFQILHD | 782 | HLA- | 9.4 | 30.44 | 1 | Tested | Yes | No |
| | c.729del | p.Phe243LeufsTer6 | | RFFNGSALNIQIIAALIK | | DRB1* | | | | | | |
| | | | | PFGQC | | 07:01 | | | | | | |
| 44 | NM_001276252.1: | NP_001263181.1: | 178 | ATYVTFSPNGTELLVN | 804 | HLA- | 4.4 | 10.01 | 6 | Tested | Yes | No |
| | c.868del | p.Glu290AsnfsTer8 | | MGGEQVYLFDLTYKQR | | A*02: | | | | | | |
| | | | | PYTFLLPR | | 01 | | | | | | |
| 27 | NM_005650.2: | NP_005641.1: | 42 | NFSVRCPKHKPPLPCPL | 787 | HLA- | 4.8 | 39.27 | 5 | Tested | Yes | No |
| | c.5826del | p. | | PPLQNKTAKGSLSTEQS | | B*07: | | | | | | |
| | | Leu1943CysfsTer118 | | ERG | | 02 | | | | | | |
| 156 | NM_016441.2: | NP_057525.1: | 5940 | CTHCYCLQGQTLCSTV | 918 | HLA- | 6 | 20.02 | 3 | Tested | Yes | No |
| | c.2567del | p. | | SCPPLPCVEPINVEGSCC | | B*15: | | | | | | |
| | | Pro856LeufsTer67 | | PMCPEM | | 17 | | | | | | |
| 37 | NM_015902.5: | NP_056986.2:p. | 119 | MSYAANLKNVMNMQN | 797 | HLA- | 6.4 | 61.73 | 4 | Tested | Yes | No |
| | c.6360del | Glu2121LysfsTer28 | | RQKKEGEEQPVLPEETE | | A*03: | | | | | | |
| | | | | SSKPGPSA | | 01 | | | | | | |
| 168 | NM_012326.2: | NP_036458.2: | 23926 | PTGPKNMQTSGRLSNV | 930 | HLA- | 6.7 | 1.00E−04 | 3 | Tested | Yes | No |
| | c.543de1 | p. | | APPCILRKNPPSARNGG | | B*15: | | | | | | |
| | | Cys182AlafsTer31 | | HETDAQI | | 17 | | | | | | |
| 20 | NM_00112 | NP_001121170.1: | 8 | GNKCTMCKEKLEREAA | 779 | HLA- | 12.2 | 461.65 | 3 | Tested | Yes | yes |
| | 7698.1:c.24 | p. | | EKKKKEDEDRSNTGER | | B*15: | | | | | | |
| | 68del | Lys823ArgfsTer119 | | SNTGERSN | | 17 | | | | | | |
| 163 | NM_00133 | NP_001317050.1: | 10387 | GAKIQWLKDAQGLPGG | 925 | HLA- | 13.3 | 37.29 | 3 | Tested | Yes | No |
| | 0121.1:c.28 | p. | | GGGDNSGTAENGRHSD | | A*02: | | | | | | |
| | 39del | Asp947ThrfsTer41 | | LAALYTIV | | 01 | | | | | | |
| 29 | NM_00131 | NP_001305049.1: | 62 | DQLQQAVQSQGFINYC | 789 | HLA- | 14.9 | 24.08 | 3 | Tested | Yes | yes |
| | 8120.1:c.13 | p. | | QKKIDASQTEFEKNVW | | A*03: | | | | | | |
| | 84del | Ile462LeufsTer16 | | SFLKVNFE | | 01 | | | | | | |
| 136 | NM_00118 | NP_001171701.1: | 8277 | SSQLLTPAERPGGLDDR | 898 | HLA- | 15.2 | 4.54 | 4 | Tested | Yes | yes |
| | 4772.2:c.52 | p. | | SPPGSSETVELVRYEPD | | B*15: | | | | | | |
| | 64del | Pro1755GlnfsTer20 | | LLRLLG | | 17 | | | | | | |
| 24 | NM_017763.4: | NP_060233.3: | 26 | FNLQKSSLSARHPQRKR | 784 | HLA- | 16.7 | 79.41 | 8 | Tested | Yes | yes |
| | c.1976del | p. | | RGGPSEPTPGSRPQDAT | | A*02: | | | | | | |
| | | Gly659ValfsTer41 | | VHPACQ | | 01 | | | | | | |
| 62 | NM_001304717.2: | NP_001291646.2: | 391 | CSIERADNDKEYLVLTL | 822 | HLA- | 17.1 | 66.83 | 3 | Tested | Yes | No |
| | c.1487del | p. | | TKNDLDKANKDKANR | | B*15: | | | | | | |
| | | Asn496MetfsTer21 | | YFSPNFKV | | 17 | | | | | | |
| 138 | NM_005116.5: | NP_005107.4: | 15302 | ESIGDYYACARLSCAPP | 900 | HLA- | 44.4 | 3.56 | 4 | Tested | Yes | No |
| | c.1233del | p.Ile412SerfsTer4 | | PPIHAINRGIFVEGLSCV | | A*02: | | | | | | |
| | | | | LDGIF | | 01 | | | | | | |
| 157 | NM_001323890.1: | NP_001310819.1: | 6022 | GTMRATGDFVTVKDGE | 919 | HLA- | 59.4 | 12.85 | 3 | Tested | Yes | No |
| | c.1319del | p. | | IFFLGRKDSQIKRHGKR | | A*02: | | | | | | |
| | | Leu440TrpfsTer43 | | LNIELVQ | | 01 | | | | | | |
| 160 | NM_005523.5: | NP_005514.1: | 7459 | LTDRQVKIWFQNRRMK | 922 | HLA- | 60.7 | 11.57 | 3 | Tested | Yes | No |
| | c.895del | p. | | EKKINRDRLQYYSANP | | A*30: | | | | | | |
| | | Ile299LeufsTer30 | | LL | | 02 | | | | | | |
| 151 | NM_030919.2: | NP_112181.2: | 4370 | SIRTTDFHNPGYPKYLG | 913 | HLA- | 141.4 | 20.3 | 3 | Tested | Yes | No |
| | c.1633del | p.His545ThrfsTer6 | | TPHLELYLSDSLRNLNK | | A*02: | | | | | | |
| | | | | ERQFHF | | 01 | | | | | | |
| 158 | NM_001321829.1: | NP_001308758.1: | 6671 | LSLEINRKLQAVLEDTL | 920 | HLA- | 347.5 | 32.63 | 3 | Tested | Yes | No |
| | c.2600del | p.Asn867IlefsTer4 | | LKNITLKENLQTLGTEI | | A*02: | | | | | | |
| | | | | ERLIKH | | 01 | | | | | | |
| 23 | NM_000918.3: | NP_000909.2: | 19 | PVKVLVGKNFEDVAFD | 783 | HLA- | 9.3 | 368.51 | 2 | Tested | Yes | No |
| | c.1160del | p. | | EKKNVFVEFYAPWCGH | | A*02: | | | | | | |
| | | Asn387ThrfsTer118 | | CKQLAPIW | | 01 | | | | | | |
| 39 | NM_001814.4: | NP_001805.3: | 144 | IIYNQGFEIVLNDYKWF | 799 | HLA- | 4.8 | 77.33 | 1 | Tested | Yes | No |
| | c.315del | p. | | AFFKYKEEGSKVTTYC | | B*15: | | | | | | |
| | | Phe105LeufsTer10 | | NETMTGW | | 03 | | | | | | |
| 57 | NM_001243439.1: | NP_001230368.1: | 345 | SFGSPTGNQMSSDIDEY | 817 | HLA- | 19.8 | 25.32 | 3 | Tested | Yes | No |
| | c.908del | p. | | KKNIHGNALRTSGSSSS | | B*15: | | | | | | |
| | | Asn303ThrfsTer63 | | DVTKAS | | 17 | | | | | | |

### F. References

The following references, to the extent that they provide exemplary procedural or other details supplementary to those set forth herein, are specifically incorporated herein by reference.
1. Lynch H T, Snyder C L, Shaw T G, Heinen C D, and Hitchins M P. Milestones of Lynch syndrome: 1895-2015. Nat Rev Cancer. 2015; 15(3):181-94.
2. Bonadona V, Bonaiti B, Olschwang S, Grandjouan S, Huiart L, Longy M, et al. Cancer risks associated with germline mutations in MLH1, MSH2, and MSH6 genes in Lynch syndrome. JAMA. 2011; 305(22):2304-10.
3. Yarchoan M, Johnson B A, 3rd, Lutz E R, Laheru D A, and Jaffee E M. Targeting neoantigens to augment antitumour immunity. Nat Rev Cancer. 2017; 17(9):569.
4. Ott P A, Hu Z, Keskin D B, Shukla S A, Sun J, Bozym D J, et al. An immunogenic personal neoantigen vaccine for patients with melanoma. Nature. 2017; 547(7662):217-21.
5. Jensen K K, Andreatta M, Marcatili P, Buus S, Greenbaum J A, Yan Z, et al. Improved methods for predicting peptide binding affinity to MHC class II molecules. Immunology. 2018; 154(3):394-406.
6. Nielsen M, and Andreatta M. NetMHCpan-3.0; improved prediction of binding to MHC class I molecules integrating information from multiple receptor and peptide length datasets. Genome Med. 2016; 8(1):33.
7. Hundal J, Carreno B M, Petti A A, Linette G P, Griffith O L, Mardis E R, et al. pVAC-Seq: A genome-guided in silico approach to identifying tumor neoantigens. Genome Med. 2016; 8(1):11.
8. Cohen C J, Gartner J J, Horovitz-Fried M, Shamalov K, Trebska-McGowan K, Bliskovsky V V, et al. Isolation of neoantigen-specific T cells from tumor and peripheral lymphocytes. J Clin Invest. 2015; 125(10):3981-91.
9. Kloor M, Reuschenbach M, Pauligk C. Karbach J, Rafiyan M R, Al-Batran S E, et al. A Frameshift Peptide Neoantigen-Based Vaccine for Mismatch Repair-Deficient Cancers: A Phase I/IIa Clinical Trial. Clin Cancer Res. 2020; 26(17):4503-10.
10. Spring K J, Zhao Z Z, Karamatic R, Walsh M D, Whitehall V L, Pike T, et al. High prevalence of sessile serrated adenomas with BRAF mutations: a prospective study of patients undergoing colonoscopy. Gastroenterology. 2006; 131(5): 1400-7.
11. Bai Y, Ni M, Cooper B, Wei Y, and Fury W. Inference of high resolution HLA types using genome-wide RNA or DNA sequencing reads. BMC Genomics. 2014; 15:325.
12. Wells D K, van Buuren M M, Dang K K, Hubbard-Lucey V M, Sheehan K C F, Campbell K M, et al. Key Parameters of Tumor Epitope Immunogenicity Revealed Through a Consortium Approach Improve Neoantigen Prediction. Cell. 2020; 183(3):818-34 e13.
13. Kloor M, Michel S, and von Knebel Doeberitz M. Immune evasion of microsatellite unstable colorectal cancers. International journal of cancer. 2010; 127(5):1001-10.
14. Chang K. Taggart M W. Reyes-Uribe L, Borras E, Riquelme E, Barnett R M, et al. Immune Profiling of Premalignant Lesions in Patients With Lynch Syndrome. JAMA Oncol. 2018.
15. Gubin M M, Artyomov M N, Mardis E R, and Schreiber R D. Tumor neoantigens: building a framework for personalized cancer immunotherapy. J Clin Invest. 2015; 125(9):3413-21.
16. Robbins P F, Lu Y C, El-Gamil M, Li Y F, Gross C, Gartner J, et al. Mining exomic sequencing data to identify mutated antigens recognized by adoptively transferred tumor-reactive T cells. Nat Med. 2013; 19(6):747-52.
17. Hause R J, Pritchard C C, Shendure J, and Salipante S J. Classification and characterization of microsatellite instability across 18 cancer types. Nat Med. 2016; 22(11):1342-50.
18. Treangen T J, and Salzberg S L. Repetitive DNA and next-generation sequencing: computational challenges and solutions. Nat Rev Genet. 2011; 13(1):36-46.
19. Vanderwalde A, Spetzler D, Xiao N, Gatalica Z, and Marshall J. Microsatellite instability status determined by next-generation sequencing and compared with P D-L1 and tumor mutational burden in 11.348 patients. Cancer Med. 2018; 7(3):746-56.
20. Ballhausen A, Przybilla M J, Jendrusch M, Haupt S, Pfaffendorf E, Seidler F, et al. The shared frameshift mutation landscape of microsatellite-unstable cancers suggests immunoediting during tumor evolution. Nat Commun. 2020; 11(1):4740.
21. Xiao Y, and Freeman G J. The microsatellite instable subset of colorectal cancer is a particularly good candidate for checkpoint blockade immunotherapy. Cancer Discov. 2015; 5(1):16-8.
22. Kloor M. and von Knebel Doeberitz M. The Immune Biology of Microsatellite-Unstable Cancer. Trends Cancer. 2016; 2(3):121-33.
23. Mardis E R. Neoantigens and genome instability: impact on immunogenomic phenotypes and immunotherapy response. Genome Med. 2019; 11(1):71.
24. Roudko V, Bozkus C C, Orfanelli T, McClain C B, Carr C, O'Donnell T, et al. Shared Immunogenic Poly-Epitope Frameshift Mutations in Microsatellite Unstable Tumors. Cell. 2020; 183(6): 1634-49 e17.
25. Wagner S, Mullins C S, and Linnebacher M. Colorectal cancer vaccines: Tumor-associated antigens vs neoantigens. World J Gastroenterol. 2018; 24(48):5418-32.
26. Maletzki C. Schmidt F, Dirks W G, Schmitt M, and Linnebacher M. Frameshift-derived neoantigens constitute immunotherapeutic targets for patients with microsatellite-instable haematological malignancies: frameshift peptides for treating MSI+ blood cancers. Eur J Cancer. 2013; 49(11):2587-95.
27. Reyes-Uribe L, Wu W, Gelincik O, Bommi P V, Francisco-Cruz A, Solis L M, et al. Naproxen chemoprevention promotes immune activation in Lynch syndrome colorectal mucosa. Gut. 2021; 70(3):555-66.
28. Van der Auwera G A, Carneiro M O, Hartl C, Poplin R, Del Angel G, Levy-Moonshine A, et al. From FastQ data to high confidence variant calls: the Genome Analysis Toolkit best practices pipeline. Curr Protoc Bioinformatics. 2013; 43:11 0 1-0 33.
29. Dobin A, Davis C A, Schlesinger F, Drenkow J, Zaleski C, Jha S, et al. STAR: ultrafast universal RNA-seq aligner. Bioinformatics. 2013; 29(1): 15-21.
30. Niu B. Ye K, Zhang Q. Lu C. Xie M, Mclellan M D, et al. MSIsensor: microsatellite instability detection using paired tumor-normal sequence data. Bioinformatics. 2014; 30(7): 1015-6.
31. Li H. A statistical framework for SNP calling, mutation discovery, association mapping and population genetical parameter estimation from sequencing data. Bioinformatics. 2011; 27(21):2987-93.
32. Li H, Handsaker B, Wysoker A, Fennell T, Ruan J, Homer N, et al. The Sequence Alignment/Map format and SAMtools. Bioinformatics. 2009; 25(16):2078-9.
33. Benjamin D, Sato T, Cibulskis K, Getz G, Stewart C, and Lichtenstein L. Calling Somatic SNVs and Indels with Mutect2. bioRxiv. 2019.
34. Maruvka Y E, Mouw K W, Karlic R, Parasuraman P, Kamburov A, Polak P, et al. Analysis of somatic microsatellite indels identifies driver events in human tumors. Nat Biotechnol. 2017; 35(10):951-9.
35. Ramos A H, Lichtenstein L, Gupta M, Lawrence M S, Pugh T J, Saksena G, et al. Oncotator: cancer variant annotation tool. Hum Mutat. 2015; 36(4):E2423-9.
36. Trivedi U H, Cezard T. Bridgett S, Montazam A, Nichols J, Blaxter M, et al. Quality control of next-generation sequencing data without a reference. Front Genet. 2014; 5:111.
37. Bolger A M, Lohse M, and Usadel B. Trimmomatic: a flexible trimmer for Illumina sequence data. Bioinformatics. 2014; 30(15):2114-20.
38. Li B, and Dewey C N. RSEM: accurate transcript quantification from RNA-Seq data with or without a reference genome. BMC Bioinformatics. 2011; 12:323.
39. Robinson M D, and Oshlack A. A scaling normalization method for differential expression analysis of RNA-seq data. Genome Biol. 2010; 11(3):R25.
40. Robinson M D, McCarthy D J, and Smyth G K. edgeR: a Bioconductor package for differential expression analysis of digital gene expression data. Bioinformatics. 2010; 26(1):139-40.
41. Chen B, Khodadoust M S, Liu C L, Newman A M, and Alizadeh A A. Profiling Tumor Infiltrating Immune Cells with CIBERSORT. Methods Mol Biol. 2018; 1711:243-59.
42. Sturm G, Finotello F, and List M. Immunedeconv: An R Package for Unified Access to Computational Methods for Estimating Immune Cell Fractions from Bulk RNA-Sequencing Data. Methods Mol Biol. 2020; 2120:223-32.
43. Subramanian A, Tamayo P, Mootha V K, Mukherjee S, Ebert B L, Gillette M A, et al. Gene set enrichment analysis: a knowledge-based approach for interpreting genome-wide expression profiles. Proc Natl Acad Sci USA. 2005; 102(43):15545-50.
44. Kanchisa M, and Goto S. KEGG: kyoto encyclopedia of genes and genomes. Nucleic Acids Res. 2000; 28(1):27-30.
45. Bechhofer D H, Kornacki J A, Firshein W, and Figurski D H. Gene control in broad host range plasmid RK2: expression, polypeptide product, and multiple regulatory functions of korB. Proc Natl Acad Sci USA. 1986; 83(2):394-8.
46. Gu Z, Eils R, and Schlesner M. Complex heatmaps reveal patterns and correlations in multidimensional genomic data. Bioinformatics. 2016; 32(18):2847-9.
47. Bai Y, Wang D, and Fury W. PHLAT: Inference of High-Resolution HLA Types from RNA and Whole Exome Sequencing. Methods Mol Biol. 2018; 1802: 193-201.
48. McLaren W. Gil L, Hunt S E, Riat H S, Ritchie G R, Thormann A, et al. The Ensembl Variant Effect Predictor. Genome Biol. 2016; 17(1):122.
49. Jurtz V, Paul S, Andreatta M, Marcatili P. Peters B, and Nielsen M. NetMHCpan-4.0: Improved Peptide-MHC Class I Interaction Predictions Integrating Eluted Ligand and Peptide Binding Affinity Data. J Immunol. 2017; 199(9):3360-8.
50. Reynisson B, Alvarez B, Paul S. Peters B, and Nielsen M. NetMHCpan-4.1 and NetMHCIIpan-4.0: improved predictions of MHC antigen presentation by concurrent motif deconvolution and integration of M S MHC eluted ligand data. Nucleic Acids Res. 2020; 48(W1):W449-W54.
51. Rasmussen M, Fenoy E, Harndahl M, Kristensen A B, Nielsen I K, Nielsen M, et al. Pan-Specific Prediction of Peptide-MHC Class I Complex Stability, a Correlate of T Cell Immunogenicity. J Immunol. 2016; 197(4): 1517-24.

All of the methods disclosed and claimed herein can be made and executed without undue experimentation in light of the present disclosure. While the compositions and methods of this invention have been described in terms of preferred embodiments or aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The references and patent applications cited herein are specifically incorporated herein by reference.

What is claimed is:

1. A peptide comprising at least 70% sequence identity to a peptide of one of SEQ ID NOS:10, 323, 221, 44, 27, 156, 37, 168, 20, 163, 29, 136, 24, 62, 138, 157, 160, 151, 158, 23, 39, or 57.

2. A peptide comprising at least 70% sequence identity to a peptide of one of SEQ ID NOS:1-776.

3. The peptide of claim 1 or 2, wherein the peptide comprises at least 6 contiguous amino acids of a peptide of one of SEQ ID NOS: 1-776.

4. The peptide of any one of claims 1-3, wherein the peptide is 15 amino acids or fewer in length.

5. The peptide of claim 4, wherein the peptide consists of 9 amino acids.

6. The peptide of claim 4, wherein the peptide consists of 15 amino acids.

7. The peptide of any one of claims 1-6, wherein the peptide is immunogenic.

8. The peptide of any one of claims 1-7, wherein the peptide is modified.

9. The peptide of claim 8, wherein the modification comprises conjugation to a molecule.

10. The peptide of claim 8 or 9, wherein the molecule comprises an antibody, a lipid, an adjuvant, or a detection moiety.

11. The peptide of any of claims 1-10, wherein the peptide has at least 90% sequence identity to a peptide of one of SEQ ID NOS:1-776.

12. The peptide of any of claims 1-11, wherein the peptide has 1, 2 or 3 substitutions relative to a peptide of one of SEQ ID NOS:1-776.

13. The peptide of any one of claims 1-11, wherein the peptide comprises 100% sequence identity to a peptide of one of SEQ ID NOS:1-776.

14. A polypeptide comprising the peptide of any one of claims 1-13.

15. The polypeptide of claim 14, wherein the polypeptide comprises at least 2 peptides of any one of claims 1-13.

16. The polypeptide of claim 14 or 15, wherein the polypeptide comprises a cell-penetrating peptide (CPP).

17. The polypeptide of claim 16, wherein the CPP comprises the Z13 variant of ZEBRA CPP Z12.

18. The polypeptide of any one of claims 14-17, wherein the polypeptide further comprises one or more TLR agonists.

19. The polypeptide of claim 18, wherein the TLR agonist comprises a TLR2, TLR4, TLR2/4 agonist, or combinations thereof.

20. The polypeptide of claim 18 or 19, wherein the TLR agonist comprises one or both of extra domain A (EDA) and Anaxa.

21. The polypeptide of any one of claims 16-20, wherein the polypeptide comprises, from amino-proximal position to carboxy-proximal position: a cell penetrating peptide, one or more peptides of claims 1-13, and a TLR agonist.

22. The polypeptide of claim 21, wherein the polypeptide further comprises a TLR agonist amino-proximal to the cell penetrating peptide.

23. A molecular complex comprising the peptide of any one of claims 1-13 and a MHC polypeptide.

24. A pharmaceutical composition comprising one or more peptide(s) or polypeptide(s) of any one of claims 1-22 or the molecular complex of claim 23 and a pharmaceutical carrier.

25. The pharmaceutical composition of claim 24, wherein the pharmaceutical composition is formulated for parenteral administration, intravenous injection, intramuscular injection, inhalation, or subcutaneous injection.

26. The pharmaceutical composition of claim 24 or 25, wherein the composition comprises at least 2 peptides.

27. The pharmaceutical composition of any one of claims 24-26, wherein the peptide is comprised in a liposome, lipid-containing nanoparticle, or in a lipid-based carrier.

28. The pharmaceutical composition of claim 27, wherein the pharmaceutical preparation is formulated for injection or inhalation as a nasal spray.

29. The pharmaceutical composition of any one of claims 24-28, wherein the composition is formulated as a vaccine.

30. The pharmaceutical composition of any one of claims 24-29, wherein the composition further comprises an adjuvant.

31. A nucleic acid encoding for the peptide or polypeptide of any one of claims 1-22.

32. The nucleic acid of claim 31, wherein the nucleic acid is DNA.

33. The nucleic acid of claim 31, wherein the nucleic acid is RNA.

34. An expression vector comprising the nucleic acid of any one of claims 31-33.

35. The expression vector of claim 34, wherein the expression vector comprises an adenoviral backbone.

36. The expression vector of claim 35, wherein the viral backbone comprises a simian adenoviral backbone.

37. A host cell comprising the nucleic acid of any one of claims 31-33 or the expression vector of any one of claims 34-36.

38. The host cell of claim 37, wherein the host cell comprises a viral packaging cell.

39. A virus produced from the host cell of claim 38.

40. An in vitro dendritic cell comprising the peptide of any one of claims 1-13, the nucleic acid of any one of claims 31-33, or the expression vector of any one of claims 34-36.

41. The dendritic cell of claim 40, wherein the dendritic cell is a mature dendritic cell.

42. The dendritic cell of claim 40 or 41, wherein the cell is a cell with an HLA-A, HLA-B, or HLA-C type.

43. A peptide-specific binding molecule, wherein the molecule specifically binds to a peptide or polypeptide of any one of claim 1-22 or the molecular complex of claim 23.

44. The binding molecule of claim 43, wherein the binding molecule is an antibody, TCR mimic antibody, scFV, camelid, aptamer, or DARPIN.

45. A method of making a cell comprising transferring the nucleic acid of any one of claims 31-33 or the expression vector of any one of claims 34-36 into the cell.

46. The method of claim 45, wherein the method further comprises isolating the expressed peptide or polypeptide.

47. A method of producing cancer-specific immune effector cells comprising:
(a) contacting a starting population of immune effector cells with a peptide or polypeptide of any one of claims 1-22 or the molecular complex of claim 23, thereby generating peptide-specific immune effector cells.

48. The method of claim 47, wherein contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface.

49. The method of claim 48, wherein the APCs are dendritic cells.

50. The method of any one of claims 47-49, wherein the immune effector cells are T cells, peripheral blood lymphocytes, NK cells, invariant NK cells, NKT cells.

51. The method of any one of claims 47-50, wherein the immune effector cells have been differentiated from mesenchymal stem cell (MSC) or induced pluripotent stem (iPS) cells.

52. The method of claim 50, wherein the T cells are CD8⁺ T cells, CD4⁺ T cells, or γδ T cells.

53. The method of claim 50, wherein the T cells are cytotoxic T lymphocytes (CTLs).

54. The method of any one of claims 47-53, wherein obtaining comprises isolating the starting population of immune effector cells from peripheral blood mononuclear cells (PBMCs).

55. The method of any one of claims 47-54, wherein the starting population of immune effector cells is obtained from a subject.

56. The method of claim 55, wherein the subject is a human.

57. The method of claim 55 or 56, wherein the subject has a cancer.

58. The method of claim 57, wherein the cancer comprises tumor cells that are positive for expression of the peptide.

59. The method of claim 58, wherein the cancer comprises leukemia, lung cancer, or skin cancer.

60. The method of any one of claims 49-59, wherein the method further comprises introducing the peptide or a nucleic acid encoding the peptide into the dendritic cells prior to the co-culturing.

61. The method of claim 60, where the peptide or nucleic acids encoding the peptide are introduced by electroporation.

62. The method of claim 60, wherein the peptide or nucleic acids encoding the peptide are introduced by adding the peptide or nucleic acid encoding the peptide to the dendritic cell culture media.

63. The method of claim 60, wherein the immune effector cells are co-cultured with a second population of dendritic cells into which the peptide or the nucleic acid encoding the peptide has been introduced.

64. The method of claim 60, wherein a population of CD8 or CD4-positive and peptide MHC tetramer-positive T cells are purified from the immune effector cells following the co-culturing.

65. The method of claim 64, wherein a clonal population of peptide-specific immune effector cells are generated by limiting or serial dilution followed by expansion of individual clones by a rapid expansion protocol.

66. The method of claim 65, wherein the method further comprises cloning of a T cell receptor (TCR) from the clonal population of peptide-specific immune effector cells.

67. The method of claim 66, wherein cloning of the TCR is cloning of a TCR alpha and a beta chain.

68. The method of claim 66 or claim 67, wherein the TCR is cloned using a 5′-Rapid amplification of cDNA ends (RACE) method.

69. The method of claim 68, wherein the cloned TCR is subcloned into an expression vector.

70. The method of claim 69, wherein the expression vector is a retroviral or lentiviral vector.

71. The method of claim 70, where a host cell is transduced with the expression vector to generate an engineered cell that expresses the TCR.

72. The method of claim 71, wherein the host cell is an immune cell.

73. The method of any one of claims 49-72, wherein the immune cell is a T cell and the engineered cell is an engineered T cell.

74. The method of claim 73, wherein the T cell is a CD8⁺ T cell, CD4+ T cell, or γδ T cell and the engineered cell is an engineered T cell.

75. The method of claim 74, wherein the starting population of immune effector cells is obtained from a subject with cancer and the host cell is allogeneic or autologous to the subject.

76. The method of claim 75, wherein the cancer is positive for expression of the peptide.

77. The method of claim 73 or 74, wherein a population of CD8 or CD4-positive and peptide MHC tetramer-positive engineered T cells are purified from the transduced host cells.

78. The method of claim 64, wherein a clonal population of peptide-specific engineered T cells are generated by limiting or serial dilution followed by expansion of individual clones by a rapid expansion protocol.

79. A peptide-specific engineered T cell produced according to any one of the methods of claim 47-58 or 71-78.

80. A pharmaceutical composition comprising the peptide-specific T cells produced according to any one of the methods of claim 47-58 or 71-78, the host cell of claim 37 or 38, or the virus of claim 39.

81. A method of treating or preventing cancer in a subject, the method comprising administering an effective amount of the peptide or polypeptide of any one of claims 1-22 or the molecular complex of claim 23, the pharmaceutical composition of any one of claim 24-30 or 80, the nucleic acid or expression vector of any one of claims 31-36, the virus of claim 39, the dendritic cell of any one of claims 40-42, or the peptide-specific T cells of claim 79 to the subject.

82. A method of stimulating an immune response in a subject, the method comprising administering an effective amount of the peptide or polypeptide of any one of claims 1-22 or the molecular complex of claim 23, the pharmaceutical composition of any one of claim 24-30 or 80, the nucleic acid or expression vector of any one of claims 31-36, the virus of claim 39, the dendritic cell of any one of claims 40-42, or the peptide-specific T cells of claim 79 to the subject.

83. The method of claim 81 or 82, wherein the subject is a human.

84. The method of any one of claims 81-83, wherein the peptide-specific T cells are autologous or allogeneic.

85. The method of any one of claims 81-84, further comprising administering at least a second therapeutic agent.

86. The method of claim 85, wherein the second therapeutic agent is an anti-cancer agent.

87. The method of any one of claims 81-86, wherein the subject has been diagnosed with cancer.

88. The method of any one of claims 81-86, wherein the subject has not been diagnosed with cancer.

89. The method of any one of claims 81-88, wherein the subject has been determined to have Lynch Syndrome.

90. The method of claim 87, wherein the cancer comprises a cancer that is positive for expression of the peptide.

91. The method of any one of claims 81-90, wherein the cancer comprises colorectal cancer.

92. The method of claim 91, wherein the colorectal cancer comprises mismatch repair deficient colorectal cancer (MMR-d) and/or microsatellite instability (MSI) positive colorectal cancer.

93. The method of any one of claims 81-92, wherein the subject is treated for stage I or stage II cancer.

94. The method of any one of claims 81-93, wherein the subject has been determined to have mismatch repair deficient colorectal cancer (MMR-d) and/or microsatellite instability (MSI) positive colorectal cancer.

95. The method of any one of claims 81-94, wherein the cancer comprises stage 0, I, II, III, or IV cancer.

96. The method of any one of claims 81-94, wherein the cancer excludes stage 0, I, II, III, or IV cancer.

97. The method of any one of claims 81-96, wherein treating comprises one or more of reducing tumor size; increasing the overall survival rate; reducing the risk of recurrence of the cancer; reducing the risk of progression; and/or increasing the chance of progression-free survival, relapse-free survival, and/or recurrence-free survival.

98. A method of cloning a peptide-specific T cell receptor (TCR), the method comprising
(a) obtaining a starting population of immune effector cells;
(b) contacting the starting population of immune effector cells with the peptide or polypeptide of any one of claims 1-22, thereby generating peptide-specific immune effector cells;
(c) purifying immune effector cells specific to the peptide, and
(d) isolating a TCR sequence from the purified immune effector cells.

99. The method of claim 98, wherein contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface.

100. The method of claim 99, wherein the APCs are dendritic cells.

101. The method of claim 98, wherein the immune effector cells are T cells, peripheral blood lymphocytes, NK cells, invariant NK cells, NKT cells.

102. The method of claim 98, wherein the immune effector cells have been differentiated from mesenchymal stem cell (MSC) or induced pluripotent stem (iPS) cells.

103. The method of claim 101, wherein the T cells are CD8⁺ T cells, CD4⁺ T cells, or γδ T cells.

104. The method of claim 101, wherein the T cells are cytotoxic T lymphocytes (CTLs).

105. The method of any one of claims 98-105, wherein obtaining comprises isolating the starting population of immune effector cells from peripheral blood mononuclear cells (PBMCs).

106. The method of any of claims 98-105, wherein the starting population of immune effector cells is obtained from a subject.

107. The method of claim 106, wherein the subject is a human.

108. The method of claim 107, wherein the subject has cancer.

109. The method of any one of claims 106-108, wherein the subject has been diagnosed with cancer.

110. The method of any one of claims 106-108, wherein the subject has not been diagnosed with cancer.

111. The method of any one of claims 106-110, wherein the subject has been determined to have Lynch Syndrome.

112. The method of claim 108, wherein the cancer comprises a cancer that is positive for expression of the peptide.

113. The method of any one of claims 106-112, wherein the cancer comprises colorectal cancer.

114. The method of claim 113, wherein the colorectal cancer comprises mismatch repair deficient colorectal cancer (MMR-d) and/or microsatellite instability (MSI) positive colorectal cancer.

115. The method of any one of claims 106-114, wherein the subject is treated for stage I or stage II cancer.

116. The method of any one of claims 113-115, wherein the subject has been determined to have mismatch repair deficient colorectal cancer (MMR-d) and/or microsatellite instability (MSI) positive colorectal cancer.

117. The method of any one of claims 106-116, wherein the cancer comprises stage 0, I, II, III, or IV cancer.

118. The method of any one of claims 106-116, wherein the cancer excludes stage 0, I, II, III, or IV cancer.

119. The method of any one of claims 100-118, wherein the method further comprises introducing the peptide or a nucleic acid encoding the peptide into the dendritic cells prior to the co-culturing.

120. The method of claim 119, where the peptide or nucleic acid encoding the peptide are introduced by electroporation.

121. The method of claim 119, wherein the peptide or nucleic acid encoding the peptide are introduced by adding the peptide or nucleic acid encoding the peptide to the media of the dendritic cells.

122. The method of claim 119, wherein the immune effector cells are co-cultured with a second population of dendritic cells into which the peptide or a nucleic acid encoding the peptide has been introduced.

123. The method of claim 119, wherein purifying is defined as purifying a population of CD4- or CD8-positive and peptide MHC tetramer-positive T cells from the immune effector cells following the co-culturing.

124. The method of claim 123, wherein the population of CD4- or CD8-positive and peptide MHC tetramer-positive T cells are purified by fluorescence activated cell sorting (FACS).

125. The method of claim 124, wherein purifying further comprises generation of a clonal population of peptide-specific immune effector cells by limiting or serial dilution of sorted cells followed by expansion of individual clones by a rapid expansion protocol.

126. The method of claim 125, wherein isolating is defined as cloning of a T cell receptor (TCR) from the clonal population of peptide-specific immune effector cells.

127. The method of any one of claims 98-126, wherein the method further comprises sequencing the TCR alpha and/or beta gene(s) and/or performing grouping of lymphocyte interactions by paratope hotspots (GLIPH) analysis.

128. The method of claim 126 or 127, wherein cloning of the TCR is cloning of a TCR alpha and a beta chain.

129. The method of claim 128, wherein the TCR alpha and beta chains are cloned using a 5′-Rapid amplification of cDNA ends (RACE) method.

130. The method of claim 129, wherein the cloned TCR is subcloned into an expression vector.

131. The method of claim 130, wherein the expression vector comprises a linker domain between the TCR alpha sequence and TCR beta sequence.

132. The method of claim 131, wherein the linker domain comprises a sequence encoding one or more peptide cleavage sites.

133. The method of claim 132, wherein the one or more cleavage sites are a Furin cleavage site and/or a P2A cleavage site.

134. The method of claim 133, wherein the TCR alpha sequence and TCR beta sequence are linked by an IRES sequence.

135. The method of any of claims 130-134, wherein the expression vector is a retroviral or lentiviral vector.

136. The method of claim 135, where a host cell is transduced with the expression vector to generate an engineered cell that expresses the TCR alpha and beta chains.

137. The method of claim 136, wherein the host cell is an immune cell.

138. A method for prognosing a patient or for detecting T cell responses in a patient, the method comprising: contacting a biological sample from the patient with the peptide or polypeptide of any one of claims 1-21 or the molecular complex of claim 23.

139. The method of claim 138, wherein the biological sample comprises a blood sample or a fraction thereof.

140. The method of claim 139, wherein the biological sample comprises lymphocytes.

141. The method of claim 140, wherein the biological sample comprises a fractionated sample comprising lymphocytes.

142. The method of any one of claims 138-141, wherein the peptide is linked to a solid support.

143. The method of claim 142, wherein the peptide is conjugated to the solid support or is bound to an antibody that is conjugated to the solid support.

144. The method of claim 142, wherein the solid support comprises a microplate, a bead, a glass surface, a slide, or a cell culture dish.

145. The method of any one of claims 138-144, wherein detecting T cell responses comprises detecting the binding of the peptide to the T cell or TCR.

146. The method of any one of claims 138-145, wherein detecting T cell responses comprises an ELISA, ELISPOT, or a tetramer assay.

147. A composition comprising at least one MHC polypeptide and the peptide of any one of claims 1-13.

148. The composition of claim 147, wherein the MHC polypeptide is and/or peptide is conjugated to a detection tag.

149. The composition of claim 147 or 148, wherein the MHC polypeptide and peptide are operatively linked to form a peptide-MHC complex.

150. The composition of claim 149, wherein the MHC polypeptide and peptide are operatively linked through a peptide bond.

151. The composition of claim 149, wherein the MHC polypeptide and peptide are operatively linked through van der Waals forces.

152. The composition of any one of claims 149-151, wherein at least two peptide-MHC complexes are operatively linked to each other.

153. The composition of claim 152, wherein at least 3 or 4 peptide-MHC complexes are operatively linked to each other.

154. The composition of any one of claims 147-153, wherein the average ratio of MHC polypeptides to peptides is 1:1 to 4:1.

155. A method comprising contacting the composition of any one of claims 148-154 with a composition comprising T cells and detecting T cells with bound peptide and/or MHC polypeptide by detecting a detection tag.

156. The method of claim 155, wherein the method further comprises counting the number of T cells bound with peptide and/or MHC.

157. The method of claim 155 or 156, wherein the composition comprising T cells is isolated from a patient having or suspected of having a cancer.

158. The method of claim 157, wherein the cancer comprises a peptide-specific cancer.

159. The method of claim 157, wherein the peptide is selected from a peptide of one of SEQ ID NOS:1-776.

160. The method of any one of claims 155-159, wherein the method further comprises sorting the number of T cells bound with peptide and/or MHC.

161. The method of claim 160, wherein the method further comprises sequencing one or more TCR genes from T cells bound with peptide and/or MHC.

162. The method of claim 161, wherein the method further comprises grouping of lymphocyte interactions by paratope hotspots (GLIPH) analysis.

163. A kit comprising the peptide or polypeptide of any one of claims 1-22 in a container.

164. The kit of claim 163, wherein the peptide is comprised in a pharmaceutical preparation.

165. The kit of claim 164, wherein the pharmaceutical preparation is formulated for parenteral administration or inhalation.

166. The kit of claim 163, wherein the peptide is comprised in a cell culture media.
